SEQUENCE LISTING (565 sequences)

SEQ ID NO: 1 - protein, 5 aa, synthetic construct
  REGION 1..5: note = TLA0015-HCDR1
  VARIANT 1: note = Xaa = Ser, Thr or Tyr
XYGVD

SEQ ID NO: 2 - protein, 5 aa, synthetic construct
  REGION 1..5: note = TLA0015-HCDR1-1
SYGVD

SEQ ID NO: 3 - protein, 5 aa, synthetic construct
  REGION 1..5: note = TLA0015-HCDR1-2
YYGVD

SEQ ID NO: 4 - protein, 5 aa, synthetic construct
  REGION 1..5: note = TLA0015-HCDR1-3
TYGVD

SEQ ID NO: 5 - protein, 16 aa, synthetic construct
  REGION 1..16: note = TLA0015-HCDR2
  VARIANT 1: note = Xaa = Arg or Val
  VARIANT 7: note = Xaa = Gly or Lys
  VARIANT 9: note = Xaa = Asp, His or Asn
  VARIANT 11: note = Xaa = Asn or Gln
  VARIANT 12: note = Xaa = Ala, Pro or Ser
XIWGFGXTXYXXALKS

SEQ ID NO: 6 - protein, 16 aa, synthetic construct
  REGION 1..16: note = TLA0015-HCDR2-1
VIWGFGGTNYNSALKS

SEQ ID NO: 7 - protein, 16 aa, synthetic construct
  REGION 1..16: note = TLA0015-HCDR2-2
VIWGFGGTNYQSALKS

SEQ ID NO: 8 - protein, 16 aa, synthetic construct
  REGION 1..16: note = TLA0015-HCDR2-3
VIWGFGGTNYNAALKS

SEQ ID NO: 9 - protein, 16 aa, synthetic construct
  REGION 1..16: note = TLA0015-HCDR2-4
VIWGFGGTNYNPALKS

SEQ ID NO: 10 - protein, 16 aa, synthetic construct
  REGION 1..16: note = TLA0015-HCDR2-5
VIWGFGGTHYNSALKS

SEQ ID NO: 11 - protein, 16 aa, synthetic construct
  REGION 1..16: note = TLA0015-HCDR2-6
RIWGFGGTNYNSALKS

SEQ ID NO: 12 - protein, 16 aa, synthetic construct
  REGION 1..16: note = TLA0015-HCDR2-7
VIWGFGKTNYNSALKS

SEQ ID NO: 13 - protein, 16 aa, synthetic construct
  REGION 1..16: note = TLA0015-HCDR2-8
VIWGFGGTDYNSALKS

SEQ ID NO: 14 - protein, 8 aa, synthetic construct
  REGION 1..8: note = TLA0015-HCDR3
  VARIANT 2: note = Xaa = Asp, Leu or Asn
  VARIANT 3: note = Xaa = Phe or Tyr
GXXDAMDY

SEQ ID NO: 15 - protein, 8 aa, synthetic construct
  REGION 1..8: note = TLA0015-HCDR3-1
GNFDAMDY

SEQ ID NO: 16 - protein, 8 aa, synthetic construct
  REGION 1..8: note = TLA0015-HCDR3-2
GNYDAMDY

SEQ ID NO: 17 - protein, 8 aa, synthetic construct
  REGION 1..8: note = TLA0015-HCDR3-3
GDFDAMDY

SEQ ID NO: 18 - protein, 8 aa, synthetic construct
  REGION 1..8: note = TLA0015-HCDR3-4
GLFDAMDY

SEQ ID NO: 19 - protein, 30 aa, synthetic construct
  REGION 1..30: note = TLA0015-HFR1
  VARIANT 2: note = Xaa = Ile or Val
  VARIANT 5: note = Xaa = Leu or Val
  VARIANT 12: note = Xaa = Ile or Val
  VARIANT 20: note = Xaa = Ile or Leu
  VARIANT 28: note = Xaa = Asp or Ser
  VARIANT 30: note = Xaa = Ile, Lys or Ser
EXQLXESGGGLXQPGGSLRXSCAVSGFXLX

SEQ ID NO: 20 - protein, 30 aa, synthetic construct
  REGION 1..30: note = TLA0015-HFR1-1
EIQLLESGGGLVQPGGSLRLSCAVSGFSLS

SEQ ID NO: 21 - protein, 30 aa, synthetic construct
  REGION 1..30: note = TLA0015-HFR1-2
EIQLLESGGGLVQPGGSLRISCAVSGFSLS

SEQ ID NO: 22 - protein, 30 aa, synthetic construct
  REGION 1..30: note = TLA0015-HFR1-3
EIQLVESGGGLVQPGGSLRISCAVSGFSLS

SEQ ID NO: 23 - protein, 30 aa, synthetic construct
  REGION 1..30: note = TLA0015-HFR1-4
EVQLLESGGGLVQPGGSLRLSCAVSGFSLS

SEQ ID NO: 24 - protein, 30 aa, synthetic construct
  REGION 1..30: note = TLA0015-HFR1-5
EIQLVESGGGLIQPGGSLRLSCAVSGFSLS

SEQ ID NO: 25 - protein, 30 aa, synthetic construct
  REGION 1..30: note = TLA0015-HFR1-6
EIQLVESGGGLIQPGGSLRISCAVSGFSLS

SEQ ID NO: 26 - protein, 30 aa, synthetic construct
  REGION 1..30: note = TLA0015-HFR1-7
EIQLVESGGGLIQPGGSLRISCAVSGFSLI

SEQ ID NO: 27 - protein, 30 aa, synthetic construct
  REGION 1..30: note = TLA0015-HFR1-8
EIQLVESGGGLIQPGGSLRISCAVSGFSLK

SEQ ID NO: 28 - protein, 30 aa, synthetic construct
  REGION 1..30: note = TLA0015-HFR1-9
EIQLVESGGGLIQPGGSLRISCAVSGFDLS

SEQ ID NO: 29 - protein, 14 aa, synthetic construct
  REGION 1..14: note = TLA0015-HFR2
  VARIANT 5: note = Xaa = Ala or Ser
  VARIANT 13: note = Xaa = Leu or Val
WVRQXPGKGLEWXG

SEQ ID NO: 30 - protein, 14 aa, synthetic construct
  REGION 1..14: note = TLA0015-HFR2-1/TLA-0238-HFR2-3
WVRQAPGKGLEWVG

SEQ ID NO: 31 - protein, 14 aa, synthetic construct
  REGION 1..14: note = TLA0015-HFR2-2
WVRQAPGKGLEWLG

SEQ ID NO: 32 - protein, 14 aa, synthetic construct
  REGION 1..14: note = TLA0015-HFR2-3
WVRQSPGKGLEWLG

SEQ ID NO: 33 - protein, 32 aa, synthetic construct
  REGION 1..32: note = TLA0015-HFR3
  VARIANT 2: note = Xaa = Phe or Leu
  VARIANT 6: note = Xaa = Ala, Lys, Arg or Val
  VARIANT 13: note = Xaa = Leu or Val
  VARIANT 32: note = Xaa = Arg or Ser
RXTISXDNSKNTXYLQMNSLRAEDTAVYYCAX

SEQ ID NO: 34 - protein, 32 aa, synthetic construct
  REGION 1..32: note = TLA0015-HFR3-1
RLTISRDNSKNTLYLQMNSLRAEDTAVYYCAS

SEQ ID NO: 35 - protein, 32 aa, synthetic construct
  REGION 1..32: note = TLA0015-HFR3-2
RLTISKDNSKNTVYLQMNSLRAEDTAVYYCAS

SEQ ID NO: 36 - protein, 32 aa, synthetic construct
  REGION 1..32: note = TLA0015-HFR3-3
RFTISRDNSKNTVYLQMNSLRAEDTAVYYCAS

SEQ ID NO: 37 - protein, 32 aa, synthetic construct
  REGION 1..32: note = TLA0015-HFR3-4
RLTISRDNSKNTVYLQMNSLRAEDTAVYYCAS

SEQ ID NO: 38 - protein, 32 aa, synthetic construct
  REGION 1..32: note = TLA0015-HFR3-5
RLTISRDNSKNTVYLQMNSLRAEDTAVYYCAR

SEQ ID NO: 39 - protein, 32 aa, synthetic construct
  REGION 1..32: note = TLA0015-HFR3-6
RLTISVDNSKNTVYLQMNSLRAEDTAVYYCAS

SEQ ID NO: 40 - protein, 32 aa, synthetic construct
  REGION 1..32: note = TLA0015-HFR3-7
RLTISADNSKNTVYLQMNSLRAEDTAVYYCAS

SEQ ID NO: 41 - protein, 11 aa, synthetic construct
  REGION 1..11: note = TLA0015-HFR4-1/TLA-0238-HFR4-2
WGQGTLVTVSS

SEQ ID NO: 42 - protein, 11 aa, synthetic construct
  REGION 1..11: note = TLA0015-LCDR1-1
RASQDISNYLN

SEQ ID NO: 43 - protein, 7 aa, synthetic construct
  REGION 1..7: note = TL0015-LCDR2-1
YTSRLHS

SEQ ID NO: 44 - protein, 9 aa, synthetic construct
  REGION 1..9: note = TL0015-LCDR3-1
QQGNTLPFT

SEQ ID NO: 45 - protein, 23 aa, synthetic construct
  REGION 1..23: note = TL0015-LFR1
  VARIANT 7: note = Xaa = Ser or Thr
  VARIANT 8: note = Xaa = Pro or Thr
  VARIANT 22: note = Xaa = Ser or Thr
DIQMTQXXSSLSASVGDRVTIXC

SEQ ID NO: 46 - protein, 23 aa, synthetic construct
  REGION 1..23: note = TL0015-LFR1-1
DIQMTQTTSSLSASLGDRVTISC

SEQ ID NO: 47 - protein, 23 aa, synthetic construct
  REGION 1..23: note = TL0015-LFR1-2/TLA0241-LFR1-1
DIQMTQSPSSLSASVGDRVTITC

SEQ ID NO: 48 - protein, 15 aa, synthetic construct
  REGION 1..15: note = TL0015-LFR2
  VARIANT 9: note = Xaa = Ala or Val
  VARIANT 10: note = Xaa = Pro or Val
WYQQKPGKXXKLLIY

SEQ ID NO: 49 - protein, 15 aa, synthetic construct
  REGION 1..15: note = TL0015-LFR2-1
WYQQKPDGTVKLLIY

SEQ ID NO: 50 - protein, 15 aa, synthetic construct
  REGION 1..15: note = TL0015-LFR2-2
WYQQKPGKVVKLLIY

SEQ ID NO: 51 - protein, 15 aa, synthetic construct
  REGION 1..15: note = TL0015-LFR2-3
WYQQKPGKAVKLLIY

SEQ ID NO: 52 - protein, 15 aa, synthetic construct
  REGION 1..15: note = TL0015-LFR2-4/TLA0241-LFR2-1
WYQQKPGKAPKLLIY

SEQ ID NO: 53 - protein, 32 aa, synthetic construct
  REGION 1..32: note = TL0015-LFR3
  VARIANT 15: note = Xaa = Phe or Tyr
  VARIANT 27: note = Xaa = Phe or Val
  VARIANT 31: note = Xaa = Phe or Tyr
GVPSRFSGSGSGTDXTLTISSLQPEDXATYXC

SEQ ID NO: 54 - protein, 32 aa, synthetic construct
  REGION 1..32: note = TL0015-LFR3-1
GVPSRFSGSGSGTDYSLTISNLEQEDIATYFC

SEQ ID NO: 55 - protein, 32 aa, synthetic construct
  REGION 1..32: note = TL0015-LFR3-2
GVPSRFSGSGSGTDYTLTISSLQPEDVATYFC

SEQ ID NO: 56 - protein, 32 aa, synthetic construct
  REGION 1..32: note = TL0015-LFR3-3
GVPSRFSGSGSGTDYTLTISSLQPEDFATYFC

SEQ ID NO: 57 - protein, 32 aa, synthetic construct
  REGION 1..32: note = TL0015-LFR3-4/TLA0238-LFR3-4/TLA0241-LFR3-2
GVPSRFSGSGSGTDFTLTISSLQPEDFATYYC

SEQ ID NO: 58 - protein, 10 aa, synthetic construct
  REGION 1..10: note = TL0015-LFR4
  VARIANT 3: note = Xaa = Pro or Gln
FGXGTKLEIK

SEQ ID NO: 59 - protein, 10 aa, synthetic construct
  REGION 1..10: note = TL0015-LFR4-1
FGSGTKLEIK

SEQ ID NO: 60 - protein, 10 aa, synthetic construct
  REGION 1..10: note = TL0015-LFR4-2
FGPGTKLEIK

SEQ ID NO: 61 - protein, 10 aa, synthetic construct
  REGION 1..10: note = TL0015-LFR4-3/TLA0238-LFR4-3
FGQGTKLEIK

SEQ ID NO: 62 - protein, 116 aa, synthetic construct
  REGION 1..116: note = TLA0015-VH-1
QIQLKESGPGLVAPSQSLSITCTVSGFSLSSYGVDWVRQSPGKGLEWLGVIWGFGGTNYNSALKSRLSISKDNSKSQVFLKMNSLQSDDSAIYYCASGNFDAMDYWGQGTSVTVSS

SEQ ID NO: 63 - protein, 116 aa, synthetic construct
  REGION 1..116: note = TLA0015-VH-2
EIQLLESGGGLVQPGGSLRLSCAVSGFSLSSYGVDWVRQAPGKGLEWVGVIWGFGGTNYNSALKSRLTISRDNSKNTLYLQMNSLRAEDTAVYYCASGNFDAMDYWGQGTLVTVSS

SEQ ID NO: 64 - protein, 116 aa, synthetic construct
  REGION 1..116: note = TLA0015-VH-3
EIQLLESGGGLVQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 65 - protein, 116 aa, synthetic construct
  REGION 1..116: note = TLA0015-VH-4
EIQLVESGGGLVQPGGSLRISCAVSGFSLSSYGVDWVRQSPGKGLEWLGVIWGFGGTNYNSALKSRLTISKDNSKNTVYLQMNSLRAEDTAVYYCASGNFDAMDYWGQGTLVTVSS

SEQ ID NO: 66 - protein, 116 aa, synthetic construct
  REGION 1..116: note = TLA0015-VH-5
EVQLLESGGGLVQPGGSLRLSCAVSGFSLSSYGVDWVRQAPGKGLEWLGVIWGFGGTNYNSALKSRFTISRDNSKNTVYLQMNSLRAEDTAVYYCASGNFDAMDYWGQGTLVTVSS

SEQ ID NO: 67 - protein, 116 aa, synthetic construct
  REGION 1..116: note = TLA0015-VH-6
EVQLLESGGGLVQPGGSLRLSCAVSGFSLSSYGVDWVRQAPGKGLEWLGVIWGFGGTNYNSALKSRLTISRDNSKNTVYLQMNSLRAEDTAVYYCASGNFDAMDYWGQGTLVTVSS

SEQ ID NO: 68 - protein, 116 aa, synthetic construct
  REGION 1..116: note = TLA0015-VH-7
EVQLLESGGGLVQPGGSLRLSCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 69 - protein, 116 aa, synthetic construct
  REGION 1..116: note = TLA0015-VH-8
EIQLVESGGGLIQPGGSLRLSCAVSGFSLSSYGVDWVRQAPGKGLEWLGVIWGFGGTNYNSALKSRLTISRDNSKNTVYLQMNSLRAEDTAVYYCARGNFDAMDYWGQGTLVTVSS

SEQ ID NO: 70 - protein, 116 aa, synthetic construct
  REGION 1..116: note = TLA0015-VH-9
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 71 - protein, 116 aa, synthetic construct
  REGION 1..116: note = TLA0015-VH-10
EIQLVESGGGLIQPGGSLRISCAVSGFSLSSYGVDWVRQAPGKGLEWLGVIWGFGGTNYNSALKSRLTISRDNSKNTVYLQMNSLRAEDTAVYYCASGNFDAMDYWGQGTLVTVSS

SEQ ID NO: 72 - protein, 116 aa, synthetic construct
  REGION 1..116: note = TLA0015-VH-11
EIQLVESGGGLIQPGGSLRISCAVSGFSLSSYGVDWVRQAPGKGLEWLGVIWGFGGTNYNSALKSRLTISVDNSKNTVYLQMNSLRAEDTAVYYCASGNFDAMDYWGQGTLVTVSS

SEQ ID NO: 73 - protein, 116 aa, synthetic construct
  REGION 1..116: note = TLA0015-VH-12
EIQLVESGGGLIQPGGSLRISCAVSGFSLSSYGVDWVRQAPGKGLEWLGVIWGFGGTNYQSALKSRLTISKDNSKNTVYLQMNSLRAEDTAVYYCASGNFDAMDYWGQGTLVTVSS

SEQ ID NO: 74 - protein, 116 aa, synthetic construct
  REGION 1..116: note = TLA0015-VH-13
EIQLVESGGGLIQPGGSLRISCAVSGFSLSSYGVDWVRQAPGKGLEWLGVIWGFGGTNYNAALKSRLTISKDNSKNTVYLQMNSLRAEDTAVYYCASGNFDAMDYWGQGTLVTVSS

SEQ ID NO: 75 - protein, 116 aa, synthetic construct
  REGION 1..116: note = TLA0015-VH-14
EIQLVESGGGLIQPGGSLRISCAVSGFSLSSYGVDWVRQAPGKGLEWLGVIWGFGGTNYNSALKSRLTISADNSKNTVYLQMNSLRAEDTAVYYCASGNFDAMDYWGQGTLVTVSS

SEQ ID NO: 76 - protein, 116 aa, synthetic construct
  REGION 1..116: note = TLA0015-VH-15
EIQLVESGGGLIQPGGSLRISCAVSGFSLSYYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 77 - protein, 116 aa, synthetic construct
  REGION 1..116: note = TLA0015-VH-16
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNYDAMDYWGQGTLVTVSS

SEQ ID NO: 78 - protein, 116 aa, synthetic construct
  REGION 1..116: note = TLA0015-VH-17
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGDFDAMDYWGQGTLVTVSS

SEQ ID NO: 79 - protein, 116 aa, synthetic construct
  REGION 1..116: note = TLA0015-VH-18
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGLFDAMDYWGQGTLVTVSS

SEQ ID NO: 80 - protein, 116 aa, synthetic construct
  REGION 1..116: note = TLA0015-VH-19
EIQLVESGGGLIQPGGSLRISCAVSGFSLSTYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 81 - protein, 116 aa, synthetic construct
  REGION 1..116: note = TLA0015-VH-20
EIQLVESGGGLIQPGGSLRISCAVSGFSLISYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 82 - protein, 116 aa, synthetic construct
  REGION 1..116: note = TLA0015-VH-21
EIQLVESGGGLIQPGGSLRISCAVSGFSLSSYGVDWVRQAPGKGLEWLGVIWGFGGTNYNPALKSRLTISKDNSKNTVYLQMNSLRAEDTAVYYCASGNFDAMDYWGQGTLVTVSS

SEQ ID NO: 83 - protein, 116 aa, synthetic construct
  REGION 1..116: note = TLA0015-VH-22
EIQLVESGGGLIQPGGSLRISCAVSGFSLKSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 84 - protein, 116 aa, synthetic construct
  REGION 1..116: note = TLA0015-VH-23
EIQLVESGGGLIQPGGSLRISCAVSGFSLSSYGVDWVRQAPGKGLEWLGVIWGFGGTHYNSALKSRLTISKDNSKNTVYLQMNSLRAEDTAVYYCASGNFDAMDYWGQGTLVTVSS

SEQ ID NO: 85 - protein, 116 aa, synthetic construct
  REGION 1..116: note = TLA0015-VH-24
EIQLVESGGGLIQPGGSLRISCAVSGFSLSSYGVDWVRQAPGKGLEWLGRIWGFGGTNYNSALKSRLTISKDNSKNTVYLQMNSLRAEDTAVYYCASGNFDAMDYWGQGTLVTVSS

SEQ ID NO: 86 - protein, 116 aa, synthetic construct
  REGION 1..116: note = TLA0015-VH-25
EIQLVESGGGLIQPGGSLRISCAVSGFDLSSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 87 - protein, 116 aa, synthetic construct
  REGION 1..116: note = TLA0015-VH-26
EIQLVESGGGLIQPGGSLRISCAVSGFSLSSYGVDWVRQAPGKGLEWLGVIWGFGKTNYNSALKSRLTISKDNSKNTVYLQMNSLRAEDTAVYYCASGNFDAMDYWGQGTLVTVSS

SEQ ID NO: 88 - protein, 116 aa, synthetic construct
  REGION 1..116: note = TLA0015-VH-27
EIQLVESGGGLIQPGGSLRISCAVSGFSLSSYGVDWVRQAPGKGLEWLGVIWGFGGTDYNSALKSRLTISKDNSKNTVYLQMNSLRAEDTAVYYCASGNFDAMDYWGQGTLVTVSS

SEQ ID NO: 89 - protein, 116 aa, synthetic construct
  REGION 1..116: note = TLA0015-VH-28
EIQLVESGGGLIQPGGSLRISCAVSGFSLSYYGVDWVRQAPGKGLEWLGVIWGFGGTNYNSALKSRLTISKDNSKNTVYLQMNSLRAEDTAVYYCASGNYDAMDYWGQGTLVTVSS

SEQ ID NO: 90 - protein, 116 aa, synthetic construct
  REGION 1..116: note = TLA0015-VH-29
EIQLVESGGGLIQPGGSLRISCAVSGFSLSYYGVDWVRQAPGKGLEWLGVIWGFGGTNYNSALKSRLTISKDNSKNTVYLQMNSLRAEDTAVYYCASGDFDAMDYWGQGTLVTVSS

SEQ ID NO: 91 - protein, 116 aa, synthetic construct
  REGION 1..116: note = TLA0015-VH-30
EIQLVESGGGLIQPGGSLRISCAVSGFSLSYYGVDWVRQAPGKGLEWLGVIWGFGGTNYNPALKSRLTISKDNSKNTVYLQMNSLRAEDTAVYYCASGNFDAMDYWGQGTLVTVSS

SEQ ID NO: 92 - protein, 116 aa, synthetic construct
  REGION 1..116: note = TLA0015-VH-31
EIQLVESGGGLIQPGGSLRISCAVSGFSLSYYGVDWVRQAPGKGLEWLGVIWGFGGTHYNSALKSRLTISKDNSKNTVYLQMNSLRAEDTAVYYCASGNFDAMDYWGQGTLVTVSS

SEQ ID NO: 93 - protein, 116 aa, synthetic construct
  REGION 1..116: note = TLA0015-VH-32
EIQLVESGGGLIQPGGSLRISCAVSGFSLSYYGVDWVRQAPGKGLEWLGRIWGFGGTNYNSALKSRLTISKDNSKNTVYLQMNSLRAEDTAVYYCASGNFDAMDYWGQGTLVTVSS

SEQ ID NO: 94 - protein, 116 aa, synthetic construct
  REGION 1..116: note = TLA0015-VH-33
EIQLVESGGGLIQPGGSLRISCAVSGFDLSYYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 95 - protein, 116 aa, synthetic construct
  REGION 1..116: note = TLA0015-VH-34
EIQLVESGGGLIQPGGSLRISCAVSGFSLISYGVDWVRQAPGKGLEWLGVIWGFGGTNYNSALKSRLTISKDNSKNTVYLQMNSLRAEDTAVYYCASGNYDAMDYWGQGTLVTVSS

SEQ ID NO: 96 - protein, 116 aa, synthetic construct
  REGION 1..116: note = TLA0015-VH-35
EIQLVESGGGLIQPGGSLRISCAVSGFSLSSYGVDWVRQAPGKGLEWLGVIWGFGGTNYNPALKSRLTISKDNSKNTVYLQMNSLRAEDTAVYYCASGNYDAMDYWGQGTLVTVSS

SEQ ID NO: 97 - protein, 116 aa, synthetic construct
  REGION 1..116: note = TLA0015-VH-36
EIQLVESGGGLIQPGGSLRISCAVSGFSLSSYGVDWVRQAPGKGLEWLGVIWGFGGTHYNSALKSRLTISKDNSKNTVYLQMNSLRAEDTAVYYCASGNYDAMDYWGQGTLVTVSS

SEQ ID NO: 98 - protein, 116 aa, synthetic construct
  REGION 1..116: note = TLA0015-VH-37
EIQLVESGGGLIQPGGSLRISCAVSGFSLSSYGVDWVRQAPGKGLEWLGRIWGFGGTNYNSALKSRLTISKDNSKNTVYLQMNSLRAEDTAVYYCASGNYDAMDYWGQGTLVTVSS

SEQ ID NO: 99 - protein, 116 aa, synthetic construct
  REGION 1..116: note = TLA0015-VH-38
EIQLVESGGGLIQPGGSLRISCAVSGFSLSSYGVDWVRQAPGKGLEWLGVIWGFGKTNYNSALKSRLTISKDNSKNTVYLQMNSLRAEDTAVYYCASGNYDAMDYWGQGTLVTVSS

SEQ ID NO: 100 - protein, 116 aa, synthetic construct
  REGION 1..116: note = TLA0015-VH-39
EIQLVESGGGLIQPGGSLRISCAVSGFSLISYGVDWVRQAPGKGLEWLGVIWGFGGTNYNSALKSRLTISKDNSKNTVYLQMNSLRAEDTAVYYCASGDFDAMDYWGQGTLVTVSS

SEQ ID NO: 101 - protein, 116 aa, synthetic construct
  REGION 1..116: note = TLA0015-VH-40
EIQLVESGGGLIQPGGSLRISCAVSGFSLSSYGVDWVRQAPGKGLEWLGVIWGFGGTHYNSALKSRLTISKDNSKNTVYLQMNSLRAEDTAVYYCASGDFDAMDYWGQGTLVTVSS

SEQ ID NO: 102 - protein, 107 aa, synthetic construct
  REGION 1..107: note = TLA0015-VL-1
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPFTFGSGTKLEIK

SEQ ID NO: 103 - protein, 107 aa, synthetic construct
  REGION 1..107: note = TLA0015-VL-2
DIQMTQSPSSLSASVGDRVTITCRASQDISNYLNWYQQKPGKVVKLLIYYTSRLHSGVPSRFSGSGSGTDYTLTISSLQPEDVATYFCQQGNTLPFTFGPGTKLEIK

SEQ ID NO: 104 - protein, 107 aa, synthetic construct
  REGION 1..107: note = TLA0015-VL-3
DIQMTQSPSSLSASVGDRVTITCRASQDISNYLNWYQQKPGKAPKLLIYYTSRLHSGVPSRFSGSGSGTDFTLTISSLQPEDFATYYCQQGNTLPFTFGQGTKLEIK

SEQ ID NO: 105 - protein, 107 aa, synthetic construct
  REGION 1..107: note = TLA0015-VL-4
DIQMTQSPSSLSASVGDRVTITCRASQDISNYLNWYQQKPGKAPKLLIYYTSRLHSGVPSRFSGSGSGTDYTLTISSLQPEDFATYFCQQGNTLPFTFGQGTKLEIK

SEQ ID NO: 106 - protein, 107 aa, synthetic construct
  REGION 1..107: note = TLA0015-VL-5
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGQGTKLEIK

SEQ ID NO: 107 - skipped

SEQ ID NO: 108 - protein, 5 aa, synthetic construct
  REGION 1..5: note = TLA-0238-HCDR1-1
NYWMN

SEQ ID NO: 109 - protein, 5 aa, synthetic construct
  REGION 1..5: note = TLA-0238-HCDR1-2
NYWMH

SEQ ID NO: 110 - protein, 5 aa, synthetic construct
  REGION 1..5: note = TLA-0238-HCDR1-3
NYWMS

SEQ ID NO: 111 - protein, 5 aa, synthetic construct
  REGION 1..5: note = TLA-0238-HCDR1-4
NYIMN

SEQ ID NO: 112 - protein, 5 aa, synthetic construct
  REGION 1..5: note = TLA-0238-HCDR1-5
NYLMN

SEQ ID NO: 113 - protein, 5 aa, synthetic construct
  REGION 1..5: note = TLA-0238-HCDR1-6
NYMMN

SEQ ID NO: 114 - protein, 5 aa, synthetic construct
  REGION 1..5: note = TLA-0238-HCDR1-7
NYYMN

SEQ ID NO: 115 - protein, 19 aa, synthetic construct
  REGION 1..19: note = TLA-0238-HCDR2
  VARIANT 15: note = Xaa = Ala, Asp or Glu
  VARIANT 16: note = Xaa = Pro or Ser
QIRLKSDNYATHYAXXVKG

SEQ ID NO: 116 - protein, 19 aa, synthetic construct
  REGION 1..19: note = TLA-0238-HCDR2-1
QIRLKSDNYATHYAESVKG

SEQ ID NO: 117 - protein, 19 aa, synthetic construct
  REGION 1..19: note = TLA-0238-HCDR2-2
QIRLKSDNYATHYADSVKG

SEQ ID NO: 118 - protein, 19 aa, synthetic construct
  REGION 1..19: note = TLA-0238-HCDR2-3
QIRLKSDNYATHYAASVKG

SEQ ID NO: 119 - protein, 19 aa, synthetic construct
  REGION 1..19: note = TLA-0238-HCDR2-4
QIRLKSDNYATHYAAPVKG

SEQ ID NO: 120 - protein, 8 aa, synthetic construct
  REGION 1..8: note = TLA-0238-HCDR3-1
LLLRYRDY

SEQ ID NO: 121 - protein, 30 aa, synthetic construct
  REGION 1..30: note = TLA-0238-HFR1
  VARIANT 1: note = Xaa = Glu or Gln
  VARIANT 3: note = Xaa = Lys or Gln
  VARIANT 4: note = Xaa = Leu or Val
  VARIANT 5: note = Xaa = Glu or Val
  VARIANT 11: note = Xaa = Leu or Val
  VARIANT 13: note = Xaa = Lys or Gln
  VARIANT 16: note = Xaa = Gly or Arg
  VARIANT 17: note = Xaa = Ala or Ser
  VARIANT 19: note = Xaa = Lys or Arg
  VARIANT 23: note = Xaa = Ala or Ile
XVXXXESGGGXVXPGXXLXLSCXASGFTFS

SEQ ID NO: 122 - protein, 30 aa, synthetic construct
  REGION 1..30: note = TLA-0238-HFR1-1
EVKVEESGGGLVQPGGSMKLSCIASGFTFS

SEQ ID NO: 123 - protein, 30 aa, synthetic construct
  REGION 1..30: note = TLA-0238-HFR1-2
EVQVVESGGGLVKPGGSLRLSCAASGFTFS

SEQ ID NO: 124 - protein, 30 aa, synthetic construct
  REGION 1..30: note = TLA-0238-HFR1-3
EVQVVESGGGLVQPGGSLRLSCIASGFTFS

SEQ ID NO: 125 - protein, 30 aa, synthetic construct
  REGION 1..30: note = TLA-0238-HFR1-4
EVQLVESGGGLVQPGGSLRLSCAASGFTFS

SEQ ID NO: 126 - protein, 30 aa, synthetic construct
  REGION 1..30: note = TLA-0238-HFR1-5
QVQLVESGGGVVQPGRSLRLSCAASGFTFS

SEQ ID NO: 127 - protein, 30 aa, synthetic construct
  REGION 1..30: note = TLA-0238-HFR1-6
EVKVEESGGGLVQPGGSLRLSCIASGFTFS

SEQ ID NO: 128 - protein, 30 aa, synthetic construct
  REGION 1..30: note = TLA-0238-HFR1-7
EVKVEESGGGLVQPGGSLKLSCIASGFTFS

SEQ ID NO: 129 - protein, 30 aa, synthetic construct
  REGION 1..30: note = TLA-0238-HFR1-8
EVKVEESGGGLVQPGGALRLSCIASGFTFS

SEQ ID NO: 130 - protein, 30 aa, synthetic construct
  REGION 1..30: note = TLA-0238-HFR1-9
EVKLEESGGGLVQPGGSLKLSCIASGFTFS

SEQ ID NO: 131 - protein, 30 aa, synthetic construct
  REGION 1..30: note = TLA-0238-HFR1-10
EVQLVESGGGLVKPGGSLRLSCAASGFTFS

SEQ ID NO: 132 - protein, 30 aa, synthetic construct
  REGION 1..30: note = TLA-0238-HFR1-11
EVKLEESGGGLVQPGGALRLSCIASGFTFS

SEQ ID NO: 133 - protein, 14 aa, synthetic construct
  REGION 1..14: note = TLA-0238-HFR2
  VARIANT 5: note = Xaa = Ala or Ser
  VARIANT 7: note = Xaa = Glu or Gly
  VARIANT 14: note = Xaa = Ala or Gly
WVRQXPXKGLEWVX

SEQ ID NO: 134 - protein, 14 aa, synthetic construct
  REGION 1..14: note = TLA-0238-HFR2-1
WVRQSPEKGLEWVA

SEQ ID NO: 135 - protein, 14 aa, synthetic construct
  REGION 1..14: note = TLA-0238-HFR2-2
WVRQAPGKGLEWVA

SEQ ID NO: 136 - protein, 32 aa, synthetic construct
  REGION 1..32: note = TLA-0238-HFR3
  VARIANT 1: note = Xaa = Asn or Arg
  VARIANT 3: note = Xaa = Ala or Thr
  VARIANT 8: note = Xaa = Asp or Asn
  VARIANT 9: note = Xaa = Ala or Ser
  VARIANT 11: note = Xaa = Asn or Ser
  VARIANT 12: note = Xaa = Ser or Thr
  VARIANT 13: note = Xaa = Ala, Leu or Val
  VARIANT 21: note = Xaa = Lys or Arg
  VARIANT 22: note = Xaa = Ala, Asp or Thr
  VARIANT 25: note = Xaa = Met or Thr
XFXISRDXXKXXXYLQMNSLXXEDXAVYYCTP

SEQ ID NO: 137 - protein, 32 aa, synthetic construct
  REGION 1..32: note = TLA-0238-HFR3-1
NFTISRDDSKSSVYLQMNNLRAEDTGIYYCTP

SEQ ID NO: 138 - protein, 32 aa, synthetic construct
  REGION 1..32: note = TLA-0238-HFR3-2
NFTISRDDSKSTVYLQMNSLKTEDTAVYYCTP

SEQ ID NO: 139 - protein, 32 aa, synthetic construct
  REGION 1..32: note = TLA-0238-HFR3-3
NFTISRDNAKSSVYLQMNSLRAEDTAVYYCTP

SEQ ID NO: 140 - protein, 32 aa, synthetic construct
  REGION 1..32: note = TLA-0238-HFR3-4
RFTISRDDSKSTVYLQMNSLKTEDTAVYYCTP

SEQ ID NO: 141 - protein, 32 aa, synthetic construct
  REGION 1..32: note = TLA-0238-HFR3-5
NFAISRDDSKSTVYLQMNSLKTEDTAVYYCTP

SEQ ID NO: 142 - protein, 32 aa, synthetic construct
  REGION 1..32: note = TLA-0238-HFR3-6
RFTISRDNSKNTLYLQMNSLRAEDTAVYYCTP

SEQ ID NO: 143 - protein, 32 aa, synthetic construct
  REGION 1..32: note = TLA-0238-HFR3-7
RFTISRDNSKNTLYLQMNSLKTEDTAVYYCTP

SEQ ID NO: 144 - protein, 32 aa, synthetic construct
  REGION 1..32: note = TLA-0238-HFR3-8
RFTISRDDSKNSLYLQMNSLRDEDTAVYYCTP

SEQ ID NO: 145 - protein, 32 aa, synthetic construct
  REGION 1..32: note = TLA-0238-HFR3-9
RFTISRDDSKNTLYLQMNSLRAEDTAVYYCTP

SEQ ID NO: 146 - protein, 32 aa, synthetic construct
  REGION 1..32: note = TLA-0238-HFR3-10
RFTISRDDSKNSLYLQMNSLRAEDMAVYYCTP

SEQ ID NO: 147 - protein, 32 aa, synthetic construct
  REGION 1..32: note = TLA-0238-HFR3-11
RFTISRDDSKNTAYLQMNSLRAEDTAVYYCTP

SEQ ID NO: 148 - protein, 32 aa, synthetic construct
  REGION 1..32: note = TLA-0238-HFR3-12
RFTISRDDSKNTLYLQMNSLKTEDTAVYYCTP

SEQ ID NO: 149 - protein, 11 aa, synthetic construct
  REGION 1..11: note = TLA-0238-HFR4
  VARIANT 6: note = Xaa = Leu, Met or Thr
  VARIANT 7: note = Xaa = Leu or Val
WGQGTXXTVSS

SEQ ID NO: 150 - protein, 11 aa, synthetic construct
  REGION 1..11: note = TLA-0238-HFR4-1
WGQGTTLTVSS

SEQ ID NO: 151 - protein, 11 aa, synthetic construct
  REGION 1..11: note = TLA-0238-HFR4-3
WGQGTLLTVSS

SEQ ID NO: 152 - protein, 11 aa, synthetic construct
  REGION 1..11: note = TLA-0238-HFR4-4
WGQGTMVTVSS

SEQ ID NO: 153 - protein, 11 aa, synthetic construct
  REGION 1..11: note = TLA-0238-HFR4-5
WGQGTTVTVSS

SEQ ID NO: 154 - protein, 10 aa, synthetic construct
  REGION 1..10: note = TLA0238-LCDR1
  VARIANT 9: note = Xaa = Leu or Met
  VARIANT 10: note = Xaa = Ala, His or Thr
SASSSVSYXX

SEQ ID NO: 155 - protein, 10 aa, synthetic construct
  REGION 1..10: note = TLA0238-LCDR1-1
SASSSVSYMH

SEQ ID NO: 156 - protein, 10 aa, synthetic construct
  REGION 1..10: note = TLA0238-LCDR1-2
SASSSVSYLH

SEQ ID NO: 157 - protein, 10 aa, synthetic construct
  REGION 1..10: note = TLA0238-LCDR1-3
SASSSVSYLA

SEQ ID NO: 158 - protein, 10 aa, synthetic construct
  REGION 1..10: note = TLA0238-LCDR1-4
SASSSVSYLT

SEQ ID NO: 159 - protein, 7 aa, synthetic construct
  REGION 1..7: note = TLA0238-LCDR2
  VARIANT 5: note = Xaa = Leu or Arg
  VARIANT 7: note = Xaa = Ser or Thr
DTSNXAX

SEQ ID NO: 160 - protein, 7 aa, synthetic construct
  REGION 1..7: note = TLA0238-LCDR2-1
DTSNLAS

SEQ ID NO: 161 - protein, 7 aa, synthetic construct
  REGION 1..7: note = TLA0238-LCDR2-2
DTSNRAS

SEQ ID NO: 162 - protein, 7 aa, synthetic construct
  REGION 1..7: note = TLA0238-LCDR2-3
DTSNLAT

SEQ ID NO: 163 - protein, 9 aa, synthetic construct
  REGION 1..9: note = TLA0238-LCDR3
  VARIANT 4: note = Xaa = Glu, Asn or Ser
  VARIANT 5: note = Xaa = Ala, Glu or Gly
FQEXXHPFT

SEQ ID NO: 164 - protein, 9 aa, synthetic construct
  REGION 1..9: note = TLA0238-LCDR3-1
FQENGHPFT

SEQ ID NO: 165 - protein, 9 aa, synthetic construct
  REGION 1..9: note = TLA0238-LCDR3-2
FQESGHPFT

SEQ ID NO: 166 - protein, 9 aa, synthetic construct
  REGION 1..9: note = TLA0238-LCDR3-3
FQENEHPFT

SEQ ID NO: 167 - protein, 9 aa, synthetic construct
  REGION 1..9: note = TLA0238-LCDR3-4
FQEEGHPFT

SEQ ID NO: 168 - protein, 9 aa, synthetic construct
  REGION 1..9: note = TLA0238-LCDR3-5
FQENAHPFT

SEQ ID NO: 169 - protein, 23 aa, synthetic construct
  REGION 1..23: note = TLA0238-LFR1
  VARIANT 1: note = Xaa = Asp or Glu
  VARIANT 2: note = Xaa = Ile, Asn, Thr or Val
  VARIANT 3: note = Xaa = Gln or Val
  VARIANT 4: note = Xaa = Leu or Met
  VARIANT 9: note = Xaa = Ala or Ser
  VARIANT 10: note = Xaa = Phe, Ile, Ser or Thr
  VARIANT 11: note = Xaa = Leu, Met or Val
  VARIANT 13: note = Xaa = Ala, Leu or Val
  VARIANT 14: note = Xaa = Ser or Thr
  VARIANT 15: note = Xaa = Pro or Val
  VARIANT 17: note = Xaa = Asp or Glu
  VARIANT 18: note = Xaa = Lys or Arg
  VARIANT 19: note = Xaa = Ala or Val
  VARIANT 21: note = Xaa = Ile or Met
  VARIANT 22: note = Xaa = Ser or Thr
XXXXTQSPXXXSXXXGXXXTXXC

SEQ ID NO: 170 - protein, 23 aa, synthetic construct
  REGION 1..23: note = TLA0238-LFR1-1
ENVLTQSPAIMSASPGEKVTMTC

SEQ ID NO: 171 - protein, 23 aa, synthetic construct
  REGION 1..23: note = TLA0238-LFR1-2
ENVLTQSPATLSLSPGERATMSC

SEQ ID NO: 172 - protein, 23 aa, synthetic construct
  REGION 1..23: note = TLA0238-LFR1-3
DNQLTQSPSSLSASVGDRVTITC

SEQ ID NO: 173 - protein, 23 aa, synthetic construct
  REGION 1..23: note = TLA0238-LFR1-4/TLA0241-LFR1-2
DIQMTQSPSSVSASVGDRVTITC

SEQ ID NO: 174 - protein, 23 aa, synthetic construct
  REGION 1..23: note = TLA0238-LFR1-5
ENVLTQSPAIMSATPGEKVTITC

SEQ ID NO: 175 - protein, 23 aa, synthetic construct
  REGION 1..23: note = TLA0238-LFR1-6
EVVLTQSPAFLSVTPGEKVTITC

SEQ ID NO: 176 - protein, 23 aa, synthetic construct
  REGION 1..23: note = TLA0238-LFR1-7
ENVLTQSPAFMSATPGEKVTITC

SEQ ID NO: 177 - protein, 23 aa, synthetic construct
  REGION 1..23: note = TLA0238-LFR1-8
EIVLTQSPAFMSATPGEKVTITC

SEQ ID NO: 178 - protein, 23 aa, synthetic construct
  REGION 1..23: note = TLA0238-LFR1-9
ETVLTQSPAFMSATPGEKVTITC

SEQ ID NO: 179 - protein, 23 aa, synthetic construct
  REGION 1..23: note = TLA0238-LFR1-10
ENVLTQSPAFLSVTPGEKVTITC

SEQ ID NO: 180 - protein, 15 aa, synthetic construct
  REGION 1..15: note = TLA0238-LFR2
  VARIANT 7: note = Xaa = Asp or Gly
  VARIANT 8: note = Xaa = Lys or Gln
  VARIANT 9: note = Xaa = Ala or Ser
  VARIANT 11: note = Xaa = Lys, Gln or Arg
WYQQKPXXXPXLWIY

SEQ ID NO: 181 - protein, 15 aa, synthetic construct
  REGION 1..15: note = TLA0238-LFR2-1
WYQQKSSTSPKLWIY

SEQ ID NO: 182 - protein, 15 aa, synthetic construct
  REGION 1..15: note = TLA0238-LFR2-2
WYQQKPGQAPRLWIY

SEQ ID NO: 183 - protein, 15 aa, synthetic construct
  REGION 1..15: note = TLA0238-LFR2-3
WYQQKPGKAPKLWIY

SEQ ID NO: 184 - protein, 15 aa, synthetic construct
  REGION 1..15: note = TLA0238-LFR2-4
WYQQKPGQSPQLWIY

SEQ ID NO: 185 - protein, 15 aa, synthetic construct
  REGION 1..15: note = TLA0238-LFR2-5
WYQQKPGQSPRLWIY

SEQ ID NO: 186 - protein, 15 aa, synthetic construct
  REGION 1..15: note = TLA0238-LFR2-6
WYQQKPGKSPKLWIY

SEQ ID NO: 187 - protein, 15 aa, synthetic construct
  REGION 1..15: note = TLA0238-LFR2-7
WYQQKPDQSPKLWIY

SEQ ID NO: 188 - protein, 15 aa, synthetic construct
  REGION 1..15: note = TLA0238-LFR2-8
WYQQKPGQSPKLWIY

SEQ ID NO: 189 - protein, 32 aa, synthetic construct
  REGION 1..32: note = TLA0238-LFR3
  VARIANT 4: note = Xaa = Ala, Asp, Gly or Ser
  VARIANT 13: note = Xaa = Asn or Thr
  VARIANT 14: note = Xaa = Asp or Ser
  VARIANT 15: note = Xaa = Phe or Tyr
  VARIANT 22: note = Xaa = Leu or Met
  VARIANT 23: note = Xaa = Glu or Gln
  VARIANT 24: note = Xaa = Ala or Pro
  VARIANT 27: note = Xaa = Ala, Phe or Val
  VARIANT 29: note = Xaa = Thr or Val
GVPXRFSGSGSGXXXTLTISSXXXEDXAXYYC

SEQ ID NO: 190 - protein, 32 aa, synthetic construct
  REGION 1..32: note = TLA0238-LFR3-1
GVPGRFSGSGSGNSYSLTISSMEAEDVATYYC

SEQ ID NO: 191 - protein, 32 aa, synthetic construct
  REGION 1..32: note = TLA0238-LFR3-2
GVPARFSGSGSGNDYTLTISSMEPEDFAVYYC

SEQ ID NO: 192 - protein, 32 aa, synthetic construct
  REGION 1..32: note = TLA0238-LFR3-3
GVPGRFSGSGSGNDYTLTISSLQPEDFATYYC

SEQ ID NO: 193 - protein, 32 aa, synthetic construct
  REGION 1..32: note = TLA0238-LFR3-5
GVPDRFSGSGSGTDYTLTISSLQPEDVATYYC

SEQ ID NO: 194 - protein, 32 aa, synthetic construct
  REGION 1..32: note = TLA0238-LFR3-6
GVPDRFSGSGSGTDYTLTISSLQPEDFAVYYC

SEQ ID NO: 195 - protein, 32 aa, synthetic construct
  REGION 1..32: note = TLA0238-LFR3-7
GVPSRFSGSGSGTSYTLTISSLQPEDVATYYC

SEQ ID NO: 196 - protein, 32 aa, synthetic construct
  REGION 1..32: note = TLA0238-LFR3-8
GVPDRFSGSGSGTDYTLTISSLEAEDAATYYC

SEQ ID NO: 197 - protein, 32 aa, synthetic construct
  REGION 1..32: note = TLA0238-LFR3-9
GVPSRFSGSGSGTDYTLTISSLQPEDFATYYC

SEQ ID NO: 198 - protein, 10 aa, synthetic construct
  REGION 1..10: note = TLA0238-LFR4
  VARIANT 3: note = Xaa = Gly or Gln
  VARIANT 7: note = Xaa = Leu or Val
  VARIANT 9: note = Xaa = Ile or Met
FGXGTKXEXK

SEQ ID NO: 199 - protein, 10 aa, synthetic construct
  REGION 1..10: note = TLA0238-LFR4-1
FGGGTKLEMK

SEQ ID NO: 200 - protein, 10 aa, synthetic construct
  REGION 1..10: note = TLA0238-LFR4-2
FGGGTKVEMK

SEQ ID NO: 201 - protein, 10 aa, synthetic construct
  REGION 1..10: note = TLA0238-LFR4-4/TLA0241-LFR4-1
FGQGTKVEIK

SEQ ID NO: 202 - protein, 119 aa, synthetic construct
  REGION 1..119: note = TLA0238-VH-1
EVKVEESGGGLVQPGGSMKLSCIASGFTFSNYWMNWVRQSPEKGLEWVAQIRLKSDNYATHYAESVKGNFTISRDDSKSSVYLQMNNLRAEDTGIYYCTPLLLRYRDYWGQGTTLTVSS

SEQ ID NO: 203 - protein, 119 aa, synthetic construct
  REGION 1..119: note = TLA0238-VH-2
EVQVVESGGGLVKPGGSLRLSCAASGFTFSNYWMNWVRQAPGKGLEWVAQIRLKSDNYATHYAESVKGNFTISRDDSKSTVYLQMNSLKTEDTAVYYCTPLLLRYRDYWGQGTLVTVSS

SEQ ID NO: 204 - protein, 119 aa, synthetic construct
  REGION 1..119: note = TLA0238-VH-3
EVQVVESGGGLVQPGGSLRLSCIASGFTFSNYWMNWVRQAPGKGLEWVAQIRLKSDNYATHYAESVKGNFTISRDNAKSSVYLQMNSLRAEDTAVYYCTPLLLRYRDYWGQGTLLTVSS

SEQ ID NO: 205 - protein, 119 aa, synthetic construct
  REGION 1..119: note = TLA0238-VH-4
EVQVVESGGGLVKPGGSLRLSCAASGFTFSNYWMNWVRQAPGKGLEWVAQIRLKSDNYATHYAESVKGRFTISRDDSKSTVYLQMNSLKTEDTAVYYCTPLLLRYRDYWGQGTLVTVSS

SEQ ID NO: 206 - protein, 119 aa, synthetic construct
  REGION 1..119: note = TLA0238-VH-5
EVQVVESGGGLVKPGGSLRLSCAASGFTFSNYWMNWVRQAPGKGLEWVAQIRLKSDNYATHYAESVKGNFAISRDDSKSTVYLQMNSLKTEDTAVYYCTPLLLRYRDYWGQGTLVTVSS

SEQ ID NO: 207 - protein, 119 aa, synthetic construct
  REGION 1..119: note = TLA0238-VH-6
QVQLVESGGGVVQPGRSLRLSCAASGFTFSNYWMNWVRQAPGKGLEWVAQIRLKSDNYATHYADSVKGRFTISRDNSKNTLYLQMNSLRAEDTAVYYCTPLLLRYRDYWGQGTMVTVSS

SEQ ID NO: 208 - protein, 119 aa, synthetic construct
  REGION 1..119: note = TLA0238-VH-7
EVKVEESGGGLVQPGGSLRLSCIASGFTFSNYWMHWVRQAPGKGLEWVGQIRLKSDNYATHYAASVKGRFTISRDNSKNTLYLQMNSLKTEDTAVYYCTPLLLRYRDYWGQGTLVTVSS

SEQ ID NO: 209 - protein, 119 aa, synthetic construct
  REGION 1..119: note = TLA0238-VH-8
EVKVEESGGGLVQPGGSLKLSCIASGFTFSNYWMHWVRQAPGKGLEWVGQIRLKSDNYATHYAASVKGRFTISRDDSKNSLYLQMNSLRDEDTAVYYCTPLLLRYRDYWGQGTLVTVSS

SEQ ID NO: 210 - protein, 119 aa, synthetic construct
  REGION 1..119: note = TLA0238-VH-9
EVKVEESGGGLVQPGGALRLSCIASGFTFSNYWMHWVRQAPGKGLEWVGQIRLKSDNYATHYAASVKGRFTISRDDSKNTLYLQMNSLRAEDTAVYYCTPLLLRYRDYWGQGTLVTVSS

SEQ ID NO: 211 - protein, 119 aa, synthetic construct
  REGION 1..119: note = TLA0238-VH-10
EVKVEESGGGLVQPGGSLRLSCIASGFTFSNYWMHWVRQAPGKGLEWVGQIRLKSDNYATHYAASVKGRFTISRDDSKNSLYLQMNSLRAEDMAVYYCTPLLLRYRDYWGQGTLVTVSS

SEQ ID NO: 212 - protein, 119 aa, synthetic construct
  REGION 1..119: note = TLA0238-VH-11
EVQLVESGGGLVQPGGSLRLSCAASGFTFSNYWMNWVRQAPGKGLEWVAQIRLKSDNYATHYADSVKGRFTISRDNSKNTLYLQMNSLRAEDTAVYYCTPLLLRYRDYWGQGTMVTVSS

SEQ ID NO: 213 - protein, 119 aa, synthetic construct
  REGION 1..119: note = TLA0238-VH-12
EVKLEESGGGLVQPGGSLKLSCIASGFTFSNYWMHWVRQAPGKGLEWVGQIRLKSDNYATHYAASVKGRFTISRDDSKNTAYLQMNSLRAEDTAVYYCTPLLLRYRDYWGQGTLVTVSS

SEQ ID NO: 214 - protein, 119 aa, synthetic construct
  REGION 1..119: note = TLA0238-VH-13
EVQLVESGGGLVKPGGSLRLSCAASGFTFSNYWMNWVRQAPGKGLEWVAQIRLKSDNYATHYAAPVKGRFTISRDDSKNTLYLQMNSLKTEDTAVYYCTPLLLRYRDYWGQGTTVTVSS

SEQ ID NO: 215 - protein, 119 aa, synthetic construct
  REGION 1..119: note = TLA0238-VH-14
EVKLEESGGGLVQPGGALRLSCIASGFTFSNYWMSWVRQAPGKGLEWVGQIRLKSDNYATHYAASVKGRFTISRDDSKNTLYLQMNSLRAEDTAVYYCTPLLLRYRDYWGQGTLVTVSS

SEQ ID NO: 216 - protein, 119 aa, synthetic construct
  REGION 1..119: note = TLA0238-VH-15
EVQLVESGGGLVQPGGSLRLSCAASGFTFSNYIMNWVRQAPGKGLEWVAQIRLKSDNYATHYADSVKGRFTISRDNSKNTLYLQMNSLRAEDTAVYYCTPLLLRYRDYWGQGTLVTVSS

SEQ ID NO: 217 - protein, 119 aa, synthetic construct
  REGION 1..119: note = TLA0238-VH-16
EVQLVESGGGLVQPGGSLRLSCAASGFTFSNYLMNWVRQAPGKGLEWVAQIRLKSDNYATHYADSVKGRFTISRDNSKNTLYLQMNSLRAEDTAVYYCTPLLLRYRDYWGQGTLVTVSS

SEQ ID NO: 218 - protein, 119 aa, synthetic construct
  REGION 1..119: note = TLA0238-VH-17
EVQLVESGGGLVQPGGSLRLSCAASGFTFSNYMMNWVRQAPGKGLEWVAQIRLKSDNYATHYADSVKGRFTISRDNSKNTLYLQMNSLRAEDTAVYYCTPLLLRYRDYWGQGTLVTVSS

SEQ ID NO: 219 - protein, 119 aa, synthetic construct
  REGION 1..119: note = TLA0238-VH-18
EVQLVESGGGLVQPGGSLRLSCAASGFTFSNYWMNWVRQAPGKGLEWVAQIRLKSDNYATHYADSVKGRFTISRDNSKNTLYLQMNSLRAEDTAVYYCTPLLLRYRDYWGQGTLVTVSS

SEQ ID NO: 220 - protein, 119 aa, synthetic construct
  REGION 1..119: note = TLA0238-VH-19
EVQLVESGGGLVQPGGSLRLSCAASGFTFSNYYMNWVRQAPGKGLEWVAQIRLKSDNYATHYADSVKGRFTISRDNSKNTLYLQMNSLRAEDTAVYYCTPLLLRYRDYWGQGTLVTVSS

SEQ ID NO: 221 - protein, 106 aa, synthetic construct
  REGION 1..106: note = TLA0238-VL-1
ENVLTQSPAIMSASPGEKVTMTCSASSSVSYMHWYQQKSSTSPKLWIYDTSNLASGVPGRFSGSGSGNSYSLTISSMEAEDVATYYCFQENGHPFTFGGGTKLEMK

SEQ ID NO: 222 - protein, 106 aa, synthetic construct
  REGION 1..106: note = TLA0238-VL-2
ENVLTQSPATLSLSPGERATMSCSASSSVSYMHWYQQKPGQAPRLWIYDTSNLASGVPARFSGSGSGNDYTLTISSMEPEDFAVYYCFQENGHPFTFGGGTKVEMK

SEQ ID NO: 223 - protein, 106 aa, synthetic construct
  REGION 1..106: note = TLA0238-VL-3
DNQLTQSPSSLSASVGDRVTITCSASSSVSYMHWYQQKPGKAPKLWIYDTSNLASGVPGRFSGSGSGNDYTLTISSLQPEDFATYYCFQENGHPFTFGQGTKLEIK

SEQ ID NO: 224 - protein, 106 aa, synthetic construct
  REGION 1..106: note = TLA0238-VL-4
ENVLTQSPAIMSATPGEKVTITCSASSSVSYLHWYQQKPGQSPQLWIYDTSNRASGVPDRFSGSGSGTDYTLTISSLQPEDVATYYCFQENGHPFTFGGGTKLEMK

SEQ ID NO: 225 - protein, 106 aa, synthetic construct
  REGION 1..106: note = TLA0238-VL-5
EVVLTQSPAFLSVTPGEKVTITCSASSSVSYLAWYQQKPGQSPRLWIYDTSNRASGVPDRFSGSGSGTDYTLTISSLQPEDFAVYYCFQENGHPFTFGGGTKLEMK

SEQ ID NO: 226 - protein, 106 aa, synthetic construct
  REGION 1..106: note = TLA0238-VL-6
ENVLTQSPAFMSATPGEKVTITCSASSSVSYLAWYQQKPGKSPKLWIYDTSNLATGVPSRFSGSGSGTSYTLTISSLQPEDVATYYCFQENGHPFTFGGGTKLEMK

SEQ ID NO: 227 - protein, 106 aa, synthetic construct
  REGION 1..106: note = TLA0238-VL-7
EIVLTQSPAFMSATPGEKVTITCSASSSVSYLHWYQQKPDQSPKLWIYDTSNRASGVPDRFSGSGSGTDYTLTISSLEAEDAATYYCFQENGHPFTFGGGTKLEMK

SEQ ID NO: 228 - protein, 106 aa, synthetic construct
  REGION 1..106: note = TLA0238-VL-8
ETVLTQSPAFMSATPGEKVTITCSASSSVSYLTWYQQKPGQSPKLWIYDTSNLASGVPDRFSGSGSGTDYTLTISSLEAEDAATYYCFQENGHPFTFGGGTKLEMK

SEQ ID NO: 229 - protein, 106 aa, synthetic construct
  REGION 1..106: note = TLA0238-VL-9
DIQMTQSPSSVSASVGDRVTITCSASSSVSYMHWYQQKPGKAPKLWIYDTSNLASGVPSRFSGSGSGTDFTLTISSLQPEDFATYYCFQENGHPFTFGQGTKVEIK

SEQ ID NO: 230 - protein, 106 aa, synthetic construct
  REGION 1..106: note = TLA0238-VL-10
ENVLTQSPAFLSVTPGEKVTITCSASSSVSYLHWYQQKPDQSPKLWIYDTSNLATGVPSRFSGSGSGTDYTLTISSLQPEDFATYYCFQENGHPFTFGGGTKLEMK

SEQ ID NO: 231 - protein, 106 aa, synthetic construct
  REGION 1..106: note = TLA0238-VL-11
DIQMTQSPSSVSASVGDRVTITCSASSSVSYMHWYQQKPGKAPKLWIYDTSNLASGVPSRFSGSGSGTDFTLTISSLQPEDFATYYCFQESGHPFTFGQGTKVEIK

SEQ ID NO: 232 - protein, 106 aa, synthetic construct
  REGION 1..106: note = TLA0238-VL-12
DIQMTQSPSSVSASVGDRVTITCSASSSVSYMHWYQQKPGKAPKLWIYDTSNLASGVPSRFSGSGSGTDFTLTISSLQPEDFATYYCFQENEHPFTFGQGTKVEIK

SEQ ID NO: 233 - protein, 106 aa, synthetic construct
  REGION 1..106: note = TLA0238-VL-13
DIQMTQSPSSVSASVGDRVTITCSASSSVSYMHWYQQKPGKAPKLWIYDTSNLASGVPSRFSGSGSGTDFTLTISSLQPEDFATYYCFQEEGHPFTFGQGTKVEIK

SEQ ID NO: 234 - protein, 106 aa, synthetic construct
  REGION 1..106: note = TLA0238-VL-14
DIQMTQSPSSVSASVGDRVTITCSASSSVSYMHWYQQKPGKAPKLWIYDTSNLASGVPSRFSGSGSGTDFTLTISSLQPEDFATYYCFQENAHPFTFGQGTKVEIK

SEQ ID NO: 235 - skipped

SEQ ID NO: 236 - protein, 5 aa, synthetic construct
  REGION 1..5: note = TLA0241-HCDR1-1
DYYLN

SEQ ID NO: 237 - protein, 5 aa, synthetic construct
  REGION 1..5: note = TLA0241-HCDR1-2
DYYMH

SEQ ID NO: 238 - protein, 17 aa, synthetic construct
  REGION 1..17: note = TLA0241-HCDR2
  VARIANT 12: note = Xaa = Ala or Asn
  VARIANT 13: note = Xaa = Asp or Gln
  VARIANT 14: note = Xaa = Lys or Ser
  VARIANT 15: note = Xaa = Phe or Val
  VARIANT 16: note = Xaa = Lys, Gln or Thr
DINPNNGRTTYXXXXXG

SEQ ID NO: 239 - protein, 17 aa, synthetic construct
  REGION 1..17: note = TLA0241-HCDR2-1
DINPNNGRTTYNQKFTG

SEQ ID NO: 240 - protein, 17 aa, synthetic construct
  REGION 1..17: note = TLA0241-HCDR2-2
DINPNNGRTTYADSVKG

SEQ ID NO: 241 - protein, 17 aa, synthetic construct
  REGION 1..17: note = TLA0241-HCDR2-3
DINPNNGRTTYNQKFQG

SEQ ID NO: 242 - protein, 10 aa, synthetic construct
  REGION 1..10: note = TLA0241-HCDR3-1
EGGYGWYFDV

SEQ ID NO: 243 - protein, 30 aa, synthetic construct
  REGION 1..30: note = TLA0241-HFR1
  VARIANT 1: note = Xaa = Glu or Gln
  VARIANT 2: note = Xaa = Ile, Leu or Val
  VARIANT 5: note = Xaa = Leu, Gln or Val
  VARIANT 6: note = Xaa = Glu or Gln
  VARIANT 9: note = Xaa = Ala, Gly or Pro
  VARIANT 10: note = Xaa = Glu or Gly
  VARIANT 11: note = Xaa = Glu, Leu or Val
  VARIANT 12: note = Xaa = Lys or Val
  VARIANT 13: note = Xaa = Lys or Gln
  VARIANT 16: note = Xaa = Ala, Gly or Arg
  VARIANT 18: note = Xaa = Leu or Val
  VARIANT 20: note = Xaa = Ile or Leu
  VARIANT 23: note = Xaa = Ala, Lys or Ser
XXQLXXSGXXXXXPGXSXRXSCXASGYTFT

SEQ ID NO: 244 - protein, 30 aa, synthetic construct
  REGION 1..30: note = TLA0241-HFR1-1
EVQLVESGGGLVQPGGSLRLSCSASGYTFT

SEQ ID NO: 245 - protein, 30 aa, synthetic construct
  REGION 1..30: note = TLA0241-HFR1-2
QVQLVESGGGVVQPGRSLRLSCAASGYTFT

SEQ ID NO: 246 - protein, 30 aa, synthetic construct
  REGION 1..30: note = TLA0241-HFR1-3
EVQLQQSGPEVKKPGASVRISCKASGYTFT

SEQ ID NO: 247 - protein, 30 aa, synthetic construct
  REGION 1..30: note = TLA0241-HFR1-4
ELQLQQSGPEVKKPGASVRISCKASGYTFT

SEQ ID NO: 248 - protein, 30 aa, synthetic construct
  REGION 1..30: note = TLA0241-HFR1-5
EVQLLESGGGLVQPGGSLRLSCAASGYTFT

SEQ ID NO: 249 - protein, 30 aa, synthetic construct
  REGION 1..30: note = TLA0241-HFR1-6
EIQLQQSGAEEKKPGASVRISCKASGYTFT

SEQ ID NO: 250 - protein, 30 aa, synthetic construct
  REGION 1..30: note = TLA0241-HFR1-7
ELQLQQSGAEVKKPGASVRISCKASGYTFT

SEQ ID NO: 251 - protein, 14 aa, synthetic construct
  REGION 1..14: note = TLA0241-HFR2
  VARIANT 3: note = Xaa = Gln or Arg
  VARIANT 8: note = Xaa = Glu, Lys or Gln
WVXQAPGXGLEWIG

SEQ ID NO: 252 - protein, 14 aa, synthetic construct
  REGION 1..14: note = TLA0241-HFR2-1
WVRQAPGKGLEWIG

SEQ ID NO: 253 - protein, 14 aa, synthetic construct
  REGION 1..14: note = TLA0241-HFR2-2
WVRQAPGQGLEWIG

SEQ ID NO: 254 - protein, 14 aa, synthetic construct
  REGION 1..14: note = TLA0241-HFR2-3
WVQQAPGKGLEWIG

SEQ ID NO: 255 - protein, 14 aa, synthetic construct
  REGION 1..14: note = TLA0241-HFR2-4
WVRQAPGEGLEWIG

SEQ ID NO: 256 - protein, 32 aa, synthetic construct
  REGION 1..32: note = TLA0241-HFR3
  VARIANT 2: note = Xaa = Phe or Val
  VARIANT 5: note = Xaa = Ser or Thr
  VARIANT 6: note = Xaa = Ala or Arg
  VARIANT 8: note = Xaa = Lys, Asn or Thr
  VARIANT 10: note = Xaa = Lys, Ser or Thr
  VARIANT 11: note = Xaa = Asn or Ser
  VARIANT 13: note = Xaa = Ala or Leu
  VARIANT 15: note = Xaa = Leu or Met
  VARIANT 16: note = Xaa = Glu or Gln
  VARIANT 17: note = Xaa = Leu or Met
  VARIANT 18: note = Xaa = Asn, Arg or Ser
  VARIANT 21: note = Xaa = Arg or Thr
  VARIANT 22: note = Xaa = Ala or Ser
  VARIANT 23: note = Xaa = Asp or Glu
  VARIANT 25: note = Xaa = Met or Thr
RXTIXXDXSXXTXYXXXXSLXXXDXAVYYCAR

SEQ ID NO: 257 - protein, 32 aa, synthetic construct
  REGION 1..32: note = TLA0241-HFR3-1
RFTISRDNSKNTLYLQMSSLRAEDTAVYYCAR

SEQ ID NO: 258 - protein, 32 aa, synthetic construct
  REGION 1..32: note = TLA0241-HFR3-2
RFTISRDNSKNTLYLQMNSLRAEDTAVYYCAR

SEQ ID NO: 259 - protein, 32 aa, synthetic construct
  REGION 1..32: note = TLA0241-HFR3-3
RVTITRDTSSSTAYMELRSLRSDDTAVYYCAR

SEQ ID NO: 260 - protein, 32 aa, synthetic construct
  REGION 1..32: note = TLA0241-HFR3-4
RVTITADKSTSTAYMELRSLRSDDTAVYYCAR

SEQ ID NO: 261 - protein, 32 aa, synthetic construct
  REGION 1..32: note = TLA0241-HFR3-5
RVTITADKSTSTAYMELRSLTSDDTAVYYCAR

SEQ ID NO: 262 - protein, 32 aa, synthetic construct
  REGION 1..32: note = TLA0241-HFR3-6
RVTITADKSTSTAYMELRSLRSDDMAVYYCAR

SEQ ID NO: 263 - protein, 11 aa, synthetic construct
  REGION 1..11: note = TLA0241-HFR4
  VARIANT 3: note = Xaa = Gln, Arg or Thr
  VARIANT 6: note = Xaa = Leu, Met or Thr
WGXGTXVTVSS

SEQ ID NO: 264 - protein, 11 aa, synthetic construct
  REGION 1..11: note = TLA0241-HFR4-1
WGRGTLVTVSS

SEQ ID NO: 265 - protein, 11 aa, synthetic construct
  REGION 1..11: note = TLA0241-HFR4-2
WGQGTMVTVSS

SEQ ID NO: 266 - protein, 11 aa, synthetic construct
  REGION 1..11: note = TLA0241-HFR4-3
WGTGTLVTVSS

SEQ ID NO: 267 - protein, 11 aa, synthetic construct
  REGION 1..11: note = TLA0241-HFR4-4
WGQGTTVTVSS

SEQ ID NO: 268 - protein, 11 aa, synthetic construct
  REGION 1..11: note = TLA0241-HFR4-5
WGTGTTVTVSS

SEQ ID NO: 269 - protein, 12 aa, synthetic construct
  REGION 1..12: note = TLA0241-LCDR1
  VARIANT 2: note = Xaa = Ala or Thr
  VARIANT 11: note = Xaa = Leu or Ser
  VARIANT 12: note = Xaa = Ala or His
SXSSSIISNYXX

SEQ ID NO: 270 - protein, 12 aa, synthetic construct
  REGION 1..12: note = TLA0241-LCDR1-1
STSSSIISNYSH

SEQ ID NO: 271 - protein, 12 aa, synthetic construct
  REGION 1..12: note = TLA0241-LCDR1-2
SASSSIISNYLH

SEQ ID NO: 272 - protein, 12 aa, synthetic construct
  REGION 1..12: note = TLA0241-LCDR1-3
SASSSIISNYSA

SEQ ID NO: 273 - protein, 12 aa, synthetic construct
  REGION 1..12: note = TLA0241-LCDR1-4
SASSSIISNYLA

SEQ ID NO: 274 - protein, 7 aa, synthetic construct
  REGION 1..7: note = TLA0241-LCDR2
  VARIANT 5: note = Xaa = Leu or Arg
KTSNXPS

SEQ ID NO: 275 - protein, 7 aa, synthetic construct
  REGION 1..7: note = TLA0241-LCDR2-1
KTSNLPS

SEQ ID NO: 276 - protein, 7 aa, synthetic construct
  REGION 1..7: note = TLA0241-LCDR2-2
KTSNRPS

SEQ ID NO: 277 - protein, 9 aa, synthetic construct
  REGION 1..9: note = TLA0241-LCDR3-1
QQGSDMPIT

SEQ ID NO: 278 - protein, 23 aa, synthetic construct
  REGION 1..23: note = TLA0241-LFR1
  VARIANT 1: note = Xaa = Ala, Asp or Glu
  VARIANT 3: note = Xaa = Gln or Val
  VARIANT 4: note = Xaa = Leu or Met
  VARIANT 9: note = Xaa = Ala, Gly, Pro, Ser or Thr
  VARIANT 10: note = Xaa = Ser or Thr
  VARIANT 11: note = Xaa = Leu or Val
  VARIANT 13: note = Xaa = Ala or Leu
  VARIANT 15: note = Xaa = Pro or Val
  VARIANT 17: note = Xaa = Asp or Glu
  VARIANT 18: note = Xaa = Lys or Arg
XIXXTQSPXXXSXSXGXXVTITC

SEQ ID NO: 279 - protein, 23 aa, synthetic construct
  REGION 1..23: note = TLA0241-LFR1-3
EIVLTQSPTTLSLSPGEKVTITC

SEQ ID NO: 280 - protein, 23 aa, synthetic construct
  REGION 1..23: note = TLA0241-LFR1-4
EIVLTQSPPTLSLSPGEKVTITC

SEQ ID NO: 281 - protein, 23 aa, synthetic construct
  REGION 1..23: note = TLA0241-LFR1-5
EIVLTQSPGTLSLSPGEKVTITC

SEQ ID NO: 282 - protein, 23 aa, synthetic construct
  REGION 1..23: note = TLA0241-LFR1-6
AIQLTQSPSSLSASVGDRVTITC

SEQ ID NO: 283 - protein, 23 aa, synthetic construct
  REGION 1..23: note = TLA0241-LFR1-7
EIVLTQSPATLSLSPGEKVTITC

SEQ ID NO: 284 - protein, 15 aa, synthetic construct
  REGION 1..15: note = TLA0241-LFR2
  VARIANT 7: note = Xaa = Asp or Gly
  VARIANT 8: note = Xaa = Lys or Gln
  VARIANT 9: note = Xaa = Ala or Ser
  VARIANT 11: note = Xaa = Lys or Gln
WYQQKPXXXPXLLIY

SEQ ID NO: 285 - protein, 15 aa, synthetic construct
  REGION 1..15: note = TLA0241-LFR2-2
WYQQKPGKSPKLLIY

SEQ ID NO: 286 - protein, 15 aa, synthetic construct
  REGION 1..15: note = TLA0241-LFR2-3
WYQQKPGQSPQLLIY

SEQ ID NO: 287 - protein, 15 aa, synthetic construct
  REGION 1..15: note = TLA0241-LFR2-4
WYQQKPDQSPKLLIY

SEQ ID NO: 288 - protein, 32 aa, synthetic construct
  REGION 1..32: note = TLA0241-LFR3
  VARIANT 4: note = Xaa = Ala, Asp or Ser
  VARIANT 14: note = Xaa = Asp, Glu or Ser
  VARIANT 20: note = Xaa = Asn or Ser
  VARIANT 23: note = Xaa = Glu or Gln
  VARIANT 24: note = Xaa = Ala or Pro
  VARIANT 27: note = Xaa = Ala or Phe
GVPXRFSGSGSGTXFTLTIXSLXXEDXATYYC

SEQ ID NO: 289 - protein, 32 aa, synthetic construct
  REGION 1..32: note = TLA0241-LFR3-1
GVPSRFSGSGSGTEFTLTISSLQPEDFATYYC

SEQ ID NO: 290 - protein, 32 aa, synthetic construct
  REGION 1..32: note = TLA0241-LFR3-3
GVPSRFSGSGSGTDFTLTINSLEAEDAATYYC

SEQ ID NO: 291 - protein, 32 aa, synthetic construct
  REGION 1..32: note = TLA0241-LFR3-4
GVPDRFSGSGSGTDFTLTINSLEAEDAATYYC

SEQ ID NO: 292 - protein, 32 aa, synthetic construct
  REGION 1..32: note = TLA0241-LFR3-5
GVPARFSGSGSGTDFTLTINSLEAEDAATYYC

SEQ ID NO: 293 - protein, 32 aa, synthetic construct
  REGION 1..32: note = TLA0241-LFR3-6
GVPSRFSGSGSGTSFTLTINSLEAEDAATYYC

SEQ ID NO: 294 - protein, 10 aa, synthetic construct
  REGION 1..10: note = TLA0241-LFR4
  VARIANT 3: note = Xaa = Ala or Gln
  VARIANT 6: note = Xaa = Lys or Arg
  VARIANT 7: note = Xaa = Leu or Val
  VARIANT 9: note = Xaa = Ile or Leu
FGXGTXXEXK

SEQ ID NO: 295 - protein, 10 aa, synthetic construct
  REGION 1..10: note = TLA0241-LFR4-2
FGAGTRLELK

SEQ ID NO: 296 - protein, 10 aa, synthetic construct
  REGION 1..10: note = TLA0241-LFR4-3
FGAGTKLELK

SEQ ID NO: 297 - protein, 119 aa, synthetic construct
  REGION 1..119: note = TLA0241-VH-1
EFQLQQSGPELVKPGASVRISCKASGYTFTDYYLNWVKQSRGESLEWIGDINPNNGRTTYNQKFTGKATWTVDKSSSTVYMELRSLTSDDSAVYYCAREGGYGWYFDVWGTGTTVTVSS

SEQ ID NO: 298 - protein, 119 aa, synthetic construct
  REGION 1..119: note = TLA0241-VH-2
EVQLVESGGGLVQPGGSLRLSCSASGYTFTDYYLNWVRQAPGKGLEWIGDINPNNGRTTYADSVKGRFTISRDNSKNTLYLQMSSLRAEDTAVYYCAREGGYGWYFDVWGRGTLVTVSS

SEQ ID NO: 299 - protein, 119 aa, synthetic construct
  REGION 1..119: note = TLA0241-VH-3
QVQLVESGGGVVQPGRSLRLSCAASGYTFTDYYLNWVRQAPGKGLEWIGDINPNNGRTTYADSVKGRFTISRDNSKNTLYLQMNSLRAEDTAVYYCAREGGYGWYFDVWGQGTMVTVSS

SEQ ID NO: 300 - protein, 119 aa, synthetic construct
  REGION 1..119: note = TLA0241-VH-4
EVQLQQSGPEVKKPGASVRISCKASGYTFTDYYMHWVRQAPGQGLEWIGDINPNNGRTTYNQKFQGRVTITRDTSSSTAYMELRSLRSDDTAVYYCAREGGYGWYFDVWGTGTTVTVSS

SEQ ID NO: 301 - protein, 119 aa, synthetic construct
  REGION 1..119: note = TLA0241-VH-5
EVQLQQSGPEVKKPGASVRISCKASGYTFTDYYMHWVQQAPGKGLEWIGDINPNNGRTTYNQKFQGRVTITADKSTSTAYMELRSLRSDDTAVYYCAREGGYGWYFDVWGTGTLVTVSS

SEQ ID NO: 302 - protein, 119 aa, synthetic construct
  REGION 1..119: note = TLA0241-VH-6
ELQLQQSGPEVKKPGASVRISCKASGYTFTDYYMHWVQQAPGKGLEWIGDINPNNGRTTYNQKFQGRVTITADKSTSTAYMELRSLTSDDTAVYYCAREGGYGWYFDVWGTGTTVTVSS

SEQ ID NO: 303 - protein, 119 aa, synthetic construct
  REGION 1..119: note = TLA0241-VH-7
EVQLLESGGGLVQPGGSLRLSCAASGYTFTDYYLNWVRQAPGKGLEWIGDINPNNGRTTYADSVKGRFTISRDNSKNTLYLQMNSLRAEDTAVYYCAREGGYGWYFDVWGQGTMVTVSS

SEQ ID NO: 304 - protein, 119 aa, synthetic construct
  REGION 1..119: note = TLA0241-VH-8
EVQLQQSGPEVKKPGASVRISCKASGYTFTDYYMHWVRQAPGEGLEWIGDINPNNGRTTYNQKFQGRVTITADKSTSTAYMELRSLRSDDTAVYYCAREGGYGWYFDVWGTGTTVTVSS

SEQ ID NO: 305 - protein, 119 aa, synthetic construct
  REGION 1..119: note = TLA0241-VH-9
QVQLVESGGGVVQPGRSLRLSCAASGYTFTDYYLNWVRQAPGKGLEWIGDINPNNGRTTYADSVKGRFTISRDNSKNTLYLQMNSLRAEDTAVYYCAREGGYGWYFDVWGQGTTVTVSS

SEQ ID NO: 306 - protein, 119 aa, synthetic construct
  REGION 1..119: note = TLA0241-VH-10
EIQLQQSGAEEKKPGASVRISCKASGYTFTDYYMHWVQQAPGKGLEWIGDINPNNGRTTYNQKFQGRVTITADKSTSTAYMELRSLRSDDTAVYYCAREGGYGWYFDVWGTGTTVTVSS

SEQ ID NO: 307 - protein, 119 aa, synthetic construct
  REGION 1..119: note = TLA0241-VH-11
ELQLQQSGAEVKKPGASVRISCKASGYTFTDYYMHWVRQAPGQGLEWIGDINPNNGRTTYNQKFQGRVTITADKSTSTAYMELRSLRSDDMAVYYCAREGGYGWYFDVWGTGTLVTVSS

SEQ ID NO: 308 - protein, 108 aa, synthetic construct
  REGION 1..108: note = TLA0241-VL-1
EIVLTQSPTTMAASPGEKITITCSTSSSIISNYSHWYQQKPGFSPKLLIYKTSNLPSGVPARFSGSGSGTSYSLTIGTMEAEDAATYYCQQGSDMPITFGAGTKLELK

SEQ ID NO: 309 - protein, 108 aa, synthetic construct
  REGION 1..108: note = TLA0241-VL-2
DIQMTQSPSSLSASVGDRVTITCSTSSSIISNYSHWYQQKPGKAPKLLIYKTSNLPSGVPSRFSGSGSGTEFTLTISSLQPEDFATYYCQQGSDMPITFGQGTKVEIK

SEQ ID NO: 310 - protein, 108 aa, synthetic construct
  REGION 1..108: note = TLA0241-VL-3
EIVLTQSPTTLSLSPGEKVTITCSASSSIISNYLHWYQQKPGKSPKLLIYKTSNLPSGVPSRFSGSGSGTDFTLTINSLEAEDAATYYCQQGSDMPITFGAGTRLELK

SEQ ID NO: 311 - protein, 108 aa, synthetic construct
  REGION 1..108: note = TLA0241-VL-4
EIVLTQSPPTLSLSPGEKVTITCSASSSIISNYLHWYQQKPGQSPQLLIYKTSNRPSGVPDRFSGSGSGTDFTLTINSLEAEDAATYYCQQGSDMPITFGAGTRLELK

SEQ ID NO: 312 - protein, 108 aa, synthetic construct
  REGION 1..108: note = TLA0241-VL-5
EIVLTQSPGTLSLSPGEKVTITCSASSSIISNYLHWYQQKPDQSPKLLIYKTSNLPSGVPSRFSGSGSGTDFTLTINSLEAEDAATYYCQQGSDMPITFGAGTKLELK

SEQ ID NO: 313 - protein, 108 aa, synthetic construct
  REGION 1..108: note = TLA0241-VL-6
AIQLTQSPSSLSASVGDRVTITCSTSSSIISNYSHWYQQKPGKAPKLLIYKTSNLPSGVPSRFSGSGSGTDFTLTISSLQPEDFATYYCQQGSDMPITFGQGTKVEIK

SEQ ID NO: 314 - protein, 108 aa, synthetic construct
  REGION 1..108: note = TLA0241-VL-7
EIVLTQSPATLSLSPGEKVTITCSASSSIISNYSAWYQQKPGKSPKLLIYKTSNLPSGVPARFSGSGSGTDFTLTINSLEAEDAATYYCQQGSDMPITFGAGTRLELK

SEQ ID NO: 315 - protein, 108 aa, synthetic construct
  REGION 1..108: note = TLA0241-VL-8
DIQMTQSPSSVSASVGDRVTITCSTSSSIISNYSHWYQQKPGKAPKLLIYKTSNLPSGVPSRFSGSGSGTDFTLTISSLQPEDFATYYCQQGSDMPITFGQGTKVEIK

SEQ ID NO: 316 - protein, 108 aa, synthetic construct
  REGION 1..108: note = TLA0241-VL-9
EIVLTQSPTTLSLSPGEKVTITCSASSSIISNYLHWYQQKPDQSPKLLIYKTSNLPSGVPSRFSGSGSGTDFTLTINSLEAEDAATYYCQQGSDMPITFGAGTKLELK

SEQ ID NO: 317 - protein, 108 aa, synthetic construct
  REGION 1..108: note = TLA0241-VL-10
EIVLTQSPPTLSLSPGEKVTITCSASSSIISNYLAWYQQKPGKSPKLLIYKTSNLPSGVPSRFSGSGSGTSFTLTINSLEAEDAATYYCQQGSDMPITFGAGTKLELK

SEQ ID NO: 318 - protein, 107 aa, synthetic construct [sequence omitted]

SEQ ID NO: 319 - protein, 103 aa, synthetic construct
  REGION 1..103: note = CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 320 - protein, 227 aa, synthetic construct
  REGION 1..227: note = Fc(L234A, L235A)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 227 aa, synthetic construct
  REGION 1..227: note = Fc(L234A, L235A, M252Y, S254T, T256E)
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 124 aa, synthetic construct
  REGION 1..124: note = RVT-3101-VH
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 323 - protein, 107 aa, synthetic construct
  REGION 1..107: note = RVT-3101-VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 324 - protein, 116 aa, synthetic construct
  REGION 1..116: note = MK-7240-VH
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSS

SEQ ID NO: 325 - protein, 106 aa, synthetic construct
  REGION 1..106: note = MK-7240-VL
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIK

SEQ ID NO: 326 - protein, 251 aa, Homo sapiens
  REGION 1..251: note = Human TL1A (M1-L251)
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 327 - protein, 180 aa, Homo sapiens
  REGION 1..180: note = Human sTL1A (L72-L251)
LKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 328 - protein, 180 aa, synthetic construct
  REGION 1..180: note = sTL1A (L72-L251)
LKGQEFAPSHQQVYAPLRADGDKPRAHLTVVRQTPTQHLKNQFPALHWEHELGLAFTKNRMNYTNKFLLIPESGDYFVYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 329 - protein, 192 aa, Mus musculus
  REGION 1..192: note = Mouse sTL1A (A61-L252)
AGQLRVPGKDCMLRAITEERSEPSPQQVYSPPRGKPRAHLTIKKQTPAPHLKNQLSALHWEHDLGMAFTKNGMKYINKSLVIPESGDYFIYSQITFRGTTSVCGDISRGRRPNKPDSITVVITKVADSYPEPARLLTGSKSVCEISNNWFQSLYLGAMFSLEEGDRLMVNVSDISLVDYTKEDKTFFGAFLL

SEQ ID NO: 330 - protein, 192 aa, Rattus norvegicus
  REGION 1..192: note = Rat sTL1A (T61-I252)
TGQLRIPGKDCMFPTVTEERSAPSAQPVYTPSRDKPKAHLTIMRQTPVPHLKNELAALHWENNLGMAFTKNRMNYTNKFLVIPESGDYFIYSQITFRGTTSECGDISRVRRPKKPDSITVVITKVADSYPEPAHLLTGTKSVCEISSNWFQPIYLGAMFSLEEGDRLMVNVSDISLVDYTKEDKTFFGAFLI

SEQ ID NO: 331 - protein, 175 aa, Homo sapiens
  REGION 1..175: note = Human DR3-ECD (Q25-Q199)
QGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQ

SEQ ID NO: 332 - protein, 119 aa, synthetic construct
EVQLVESGGGLVKPGGSLRLSCAASGFTFSNYWMNWVRQAPGKGLEWVGQIRLKSDNYATHYAESVKGRFTISRDDSKNTLYLQMNSLKTEDTAVYYCTTLLLRYRDYWGQGTLVTVSS

SEQ ID NO: 333 - protein, 119 aa, synthetic construct
EVQLVESGGGLVKPGGSLRLSCAASGFTFSNYWMNWVRQAPGKGLEWVGQIRLKSDNYATHYAESVKGRFTISRDDSKNTLYLQMNSLKTEDTAVYYCTPLLLRYRDYWGQGTLVTVSS

SEQ ID NO: 334 - protein, 119 aa, synthetic construct
EVQLVESGGGLVKPGGSLRLSCAASGFTFSNYWMNWVRQAPGKGLEWVAQIRLKSDNYATHYAESVKGRFTISRDDSKNTLYLQMNSLKTEDTAVYYCTPLLLRYRDYWGQGTLVTVSS

SEQ ID NO: 335 - protein, 106 aa, synthetic construct
EIVLTQSPATLSLSPGERATLSCSASSSVSYMHWYQQKPGQSPRLWIYDTSNLASGIPARFSGSGSGTDYTLTISSLEPEDFAVYYCFQENAHPFTFGGGTKVEIK

SEQ ID NO: 336 - protein, 106 aa, synthetic construct
EIVLTQSPATLSLSPGERATLSCSASSSVSYMHWYQQKPGQSPRLWIYDTSNLASGVPARFSGSGSGTDYTLTISSLEPEDFAVYYCFQENAHPFTFGGGTKVEIK

SEQ ID NO: 337 - protein, 106 aa, synthetic construct
DIQMTQSPSSLSASVGDRVTITCSASSSVSYMHWYQQKPGKAPKLWIYDTSNLASGVPSRFSGSGSGTDYTLTISSLQPEDFATYYCFQENAHPFTFGQGTKLEIK

SEQ ID NO: 338 - protein, 106 aa, synthetic construct
DIQLTQSPSSLSASVGDRVTITCSASSSVSYMHWYQQKPGKAPKLWIYDTSNLASGVPSRFSGSGSGTDYTLTISSLQPEDFATYYCFQENAHPFTFGQGTKLEIK

SEQ ID NO: 339 - protein, 446 aa, synthetic construct
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 340 - protein, 214 aa, synthetic construct
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 341 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 342 - protein, 119 aa, synthetic construct
  REGION 1..119: note = mice-1
QIQLVQSGPELKKPGETVKISCKASGYTFTTYGMNWVKQSPGKGLKWMGWINTYSGVPTYADDFKGRFAFSLETSVNTAYLQINSLKTDDTATYFCAKDAYDNYAMDYWGQGTSVTVSS

SEQ ID NO: 343 - protein, 116 aa, synthetic construct; identical to SEQ ID NO: 62
  REGION 1..116: note = mice-2

SEQ ID NO: 344 - protein, 117 aa, synthetic construct
  REGION 1..117: note = mice-3
DVQLQESGPGLVKPSQSLSLTCSVTDYSITSGYYWNWIRQFPGNKLEWMGYIHFDGSNDYNPSLKNRISITRDTSRNQFFLKLNSVTSEDTATYYCAREGTSYFDYWGQGTTLTVSS

SEQ ID NO: 345 - protein, 115 aa, synthetic construct
  REGION 1..115: note = mice-4
QIQLVQSGPELKKPGETVKISCKASGYTFTTYGMSWVKQAPGKYLKWMGWIDTYSGVPTYADDFKGRFAFSLETSANTAYLQINNLKNEDTATYFCANSFQFAYWGQGTLVTVSA

SEQ ID NO: 346 - protein, 117 aa, synthetic construct
  REGION 1..117: note = mice-5
DVQLQESGPGLVKPSQSLSLTCSVTGYSITSGYYWNWIRQFPGNKLEWMGCISYDGSNYYNPSLKNRISFTRDTSKNQFFLKLNSVTTDDTATYYCVREGTSYVDYWGQGTTLTVSS

SEQ ID NO: 347 - protein, 117 aa, synthetic construct
  REGION 1..117: note = mice-6
DVQLQESGPGLVKPSQSLSLTCSVTDYSITSGYYWNWIRQFPGNKLEWMGYIHYDGSNDYNPSLKNRISITRDTSRNQFFLKLNSVTSEDTATYYCAREGTSYFDYWGQGTTLTVSS

SEQ ID NO: 348 - protein, 128 aa, synthetic construct
  REGION 1..128: note = mice-7
QVQLQQSGPELVRPGASVKISCKAPGYTFTSHWMQWVRQRPGQGLEWIGEIFPGSGTTYYNEKFQGKATLTVDTSSSTAYMQLSSLTSEDSSVHFCARGEGIYYPARSYGPLGYFDVWGTGTTVTVSS

SEQ ID NO: 349 - protein, 119 aa, synthetic construct
  REGION 1..119: note = mice-8
QVQLQQPGAELVMPGASVKLSCKASGYTFTNYWMNWVKQRPRQGLEWIGETDPSNTYINYNQKFKGKATLTVDKSSNTAYMQLNSLTSEDSAVYYCARGGLPYYGMDYWGQGTSVTVSS

SEQ ID NO: 350 - protein, 115 aa, synthetic construct
  REGION 1..115: note = mice-9
QIQLVQSGPELKKPGETVKISCKASGSAFTTYGMSWVKQAPGEGLKWMGYIITYSGVPTYADDFKGRFAFSLETSASTAYLQINNLKNEDTATYFCTNFYYFDYWGQGTTLTVSS

SEQ ID NO: 351 - protein, 121 aa, synthetic construct
  REGION 1..121: note = mice-10
QIQLVQSGPELKKPGETVKISCKASGYTFTTFGMNWVKQAPGKDLKWMGWINTNSGVPTYADDFRGRFAFSMETSATTAYLQINNLKNEDTATYFCARGNWEGPGIWFAYWGQGTLVTVSA

SEQ ID NO: 352 - protein, 115 aa, synthetic construct
  REGION 1..115: note = mice-11
QVQLQQPGTELVKPGASVKLSCKASGYTFTSYWMHWVKQRPGQGLEWIGNIDPNNDVINYNDKFKSKATLTVDKSSSTAYMQLSSLTSEDSGVYYCARGIYAGYWGQGTLVTVSA

SEQ ID NO: 353 - protein, 121 aa, synthetic construct
  REGION 1..121: note = mice-12
EVQLQQSGAELMKPGASVKLSCKATGYTFTGYWLEWVKQRPGHGLEWIGETLPGSGYNKYNEKFKGKATFTADTSSNTAHMQLSSLTTEDSAIYYCARRGSNYPYWYFDVWGTGTTVTVSS

SEQ ID NO: 354 - protein, 117 aa, synthetic construct
  REGION 1..117: note = mice-13
QVQLKESGPGLVQPSQSLSITCTVSGFSLTSYGVHWVRQSPGKGLEWLGVIWSGGSTDYNAAFISRLSISKDNSKSQVFFKMNSLQADDTAIYYCARNDYDYAMDYWGQGTSVTVSS

SEQ ID NO: 355 - protein, 116 aa, synthetic construct
  REGION 1..116: note = mice-14
QVQLQQSGPELVKPGASVKLSCKASGYTFTSIDINWVKQRPGQGLEWIGWIYPRDGRTDYNEKFKGKATLTVDTSSSTAYMELHSLTSEDSAVYFCARYTSSLDYWGQGTTLTVSS

SEQ ID NO: 356 - protein, 119 aa, synthetic construct
  REGION 1..119: note = mice-15
EVKLEESGGGLVQPGGSMKLSCVASGFTFSNYWMNWVRQSPEKGLEWVAQIRLKSDNYATHYAESVKGRFTISRDDSKSNVYLQMNNLRAEDTGIYYCTPLLLRSIDYWGQGTTLTVSS

SEQ ID NO: 357 - protein, 116 aa, synthetic construct
  REGION 1..116: note = mice-16
EVQLQQSGPELVKPGASVKLSCKASGYTFTSIDINWVKQRPGQGLEWIGWIYPRDGRTDYNEKFKGKATLTVDTSSSTAYMELHSLTSEDSAVYFCARYGSSLDYWGQGTTLTVSS

SEQ ID NO: 358 - protein, 116 aa, synthetic construct
  REGION 1..116: note = mice-17
QVQLQQSGPELVKPGASVKLSCKASGYTFTNQDMNWVKQRPGQGLEWIGWIFPRDGRTHHNENFKGKATLTVDTSSSTAYMELHSLTAEDSAVYFCAIYGFAMDQWGQGTSVTVSS

SEQ ID NO: 359 - protein, 116 aa, synthetic construct
  REGION 1..116: note = mice-18
QVQLQQPGPELVKPGASVKLSCKASGYTFTNQDINWVKQRPGQGLEWIGWIFPRDGRTHYNENFKGKATLTVDTSSSTAYMELHSLTAEDSAVYFCAIYGIALDHWGQGTSVTVSS

SEQ ID NO: 360 - protein, 116 aa, synthetic construct
  REGION 1..116: note = mice-19
QVQLQQPGPELVKPGASVKLSCKASGYTFTNQDINWVKQRPGQGLEWIGWIFPRDGRTHYNENFKGKATLTVDTSSSTAYMELHSLTAEDSAVYFCAIYGAAMDHWGQGTSVTVSS

SEQ ID NO: 361 - protein, 119 aa, synthetic construct
  REGION 1..119: note = mice-20
EVKLEESGGGLVQPGGSMKLSCVASGFTFSNYWMNWVRQSPEKGLEWVAQIRLKSDNYATHYAESVKGRFTISRDDSKSSVYLQMNNLRAEDTGIYYCTPLLLRYFDVWGTGTTVTVSS

SEQ ID NO: 362 - protein, 114 aa, synthetic construct
  REGION 1..114: note = mice-21
EVQLQQSVAELVRPGASVKLSCTASGFNIEDTYIHWVKQRPEQGLEWIGRIDPAHDKTKYAPKFQGKATITADTSSNTAYLQLSSLTSEDTAIYYCASTGPDWWGQGTTLTVSS

SEQ ID NO: 363 - protein, 122 aa, synthetic construct
  REGION 1..122: note = mice-22
QVQLQQSGAELVRPGTSVKMSCKASGYTFTNYWIGWTKQRPGHGLEWIGDIYPGGGYINYNEKFKGKATLTADKSSSTAYMQFSSLTSEDSAIYYCAREGGYGSGQAWFPYWGQGTLVTVSA

SEQ ID NO: 364 - protein, 118 aa, synthetic construct
  REGION 1..118: note = mice-23
QVQLQQPGTELVRPGTSVKLSCKASGYTFTSYWMHWVKQRPGQGLEWIGVIEPSDSYTDYNQKFKGKATLTVDTSSSTAYMQLSSLTSEDSAVYYCARGDYDEVFAYWGQGTLVTVSA

SEQ ID NO: 365 - protein, 118 aa, synthetic construct
  REGION 1..118: note = mice-24
QVQLQQPGAELVRPGTSVKLSCKASGYTFTSYWMHWVKQRPGQGLEWIGVIEPSDSYTDYDQKFKGKATLTVDTSSNTAYMQLSSLTSEDSAVYYCSRGDYDEVFAYWGQGTLVTVST

SEQ ID NO: 366 - protein, 119 aa, synthetic construct
  REGION 1..119: note = mice-25
QVQLQQSGADLVRPGASVTLSCKASGYTFSDSEIHWVRQTPVHGLEWIGAIDPETGDIAYNQMFKDKATLTADKSSSTAYVELRSLTSEDSAVYYCTRYGGNSWHFDVWGTGTTVTVSS

SEQ ID NO: 367 - protein, 119 aa, synthetic construct
  REGION 1..119: note = mice-26
EFQLQQSGPELLKPGASVKISCKASGYSFTDYNINWVKQRNGKSLEWIGMINPKYGITDYNQKFKGKATLTVDQFSSTAYMQVNSLTSDDSAVYYCAREELGHWCFDVWGTGTTVTVSS

SEQ ID NO: 368 - protein, 118 aa, synthetic construct
  REGION 1..118: note = mice-27
QVQLQQSGAELVRPGTSVKVSCKASGYAFTNYLIEWVNQRPGQGLEWIGVINPGSGGTKYNEKFKGKATLTADKSSSTAYMQLSSLTSEDSAVYFCAREGSAGPFDYWGQGTTLTVSS

SEQ ID NO: 369 - protein, 119 aa, synthetic construct
  REGION 1..119: note = mice-28
EFQLQQSGPELVKPGASVKISCKASGYSFTDYNINWVKQRNGKSLEWIGMINPKYGITDYNQKFKGKATLTVDQFSSTAYMQVNSLTSDDSAVYYCAREELGHWCFDVWGTGTTVTVSS

SEQ ID NO: 370 - protein, 115 aa, synthetic construct
  REGION 1..115: note = mice-29
EVQLQQSGPVLVKPGASVKMSCEASGYTLTDYYMNWVKQSHGKSLEWIGIINPYNGASNFNQRFRGKASLTFGKSSNTAYMELNSLTSEDSAVYYCASDWDVGYWGQGTALTVSS

SEQ ID NO: 371 - protein, 119 aa, synthetic construct
  REGION 1..119: note = mice-30
EVQLVESGGDLVKPGGSLKLSCAASGFTFSRYGMSWVRQTPDKRLEWVATISSGGSDTYYPDSVKGRFTISRDNAKNTLYLQMSSLKSEDTAMYYCARHGYDYGHFDYWGQGTTLTVSS

SEQ ID NO: 372 - protein, 119 aa, synthetic construct
  REGION 1..119: note = mice-31
EFQLQQSGPELVKPGASVKISCKASGYSFTDYNINWVKQSNGKSLEWIGVINPNYGITDYNQKFKGKATLTVDQSSSTAYMQLNSLTSEDSAVYYCAREELGHWCFDVWGTGTTVTVSS

SEQ ID NO: 373 - protein, 114 aa, synthetic construct
  REGION 1..114: note = mice-32
QVTLKESGPGILQPSQTLNLTCSFSGFSLSTFGMGVGWIRQPSGEGLEWLAHIWWDDHEYYNPALKSRLTISKDTSKNQVFLKIANVDTADTATYYCNRGFVYWGQGTLVTVSA

SEQ ID NO: 374 - protein, 116 aa, synthetic construct
  REGION 1..116: note = mice-33
QVQLQQSGPELVKPGTSVKLSCKASGYTFTSYDINWMKQRPGQGLEWIGWIFPRDGITKYNEKFKDKATLTVDTSSSAAYMELHSLTSEDSAVYFCARYGPAMDYWGQGTSVTVSS

SEQ ID NO: 375 - protein, 116 aa, synthetic construct
  REGION 1..116: note = mice-34
QIQLVQSGPELVKPGASVKVSCKASGYTFTRQDINWVKQRPGQGLEWIGWIYPRDGRTEYNEKFKGKVTLTVDTSSSTAYMELHSLTSEDSAVYFCATYGSSLDNWGQGTTLTVSS

SEQ ID NO: 376 - protein, 116 aa, synthetic construct
  REGION 1..116: note = mice-35
EVQLQQSGPELVKPGASVKVSCKASGYTFTRQDINWVKQRPGQGLEWIGWIYPRDGSTKYNEKFKGKVTLTVDTSSSTAYMELHSLTSEDSAVYFCATYGSSLDNWGQGTTLTVSS

SEQ ID NO: 377 - protein, 116 aa, synthetic construct
  REGION 1..116: note = mice-36
EVQLQQSGPELVKPGASVKLSCKASGYTFTNQDINWVKQRPGQGLEWIGWIFPRDGRTHYNENFKGKATLTVDTSSSTAYMELHSLTAEDSAVYFCAIYGIAMDHWGQGTSVTVSS

SEQ ID NO: 378 - protein, 116 aa, synthetic construct
  REGION 1..116: note = mice-37
QIQLVQSGPELVKPGASVKLSCKASGYTFTNYDINWVKQRPGQGLEWIGWIYPRDGSTYYNEKFKGKATLTVDTSSTTAYMEFHSLTSEDSAVYFCARYAYALNSWGQGTSVTVSS

SEQ ID NO: 379 - protein, 116 aa, synthetic construct
  REGION 1..116: note = mice-38
QVQLKQSGPELVRPGASVKLSCKASGYSFTSGDINWVKQRPGQGLEWIAWIYPRDGSTEYNEKFKGKATLTVDTSSSTAYMELHSLTSEDSAVYFCARYDCAFVYWGQGTLVTVSA

SEQ ID NO: 380 - protein, 116 aa, synthetic construct
  REGION 1..116: note = mice-39
QVQLQQSGPELVKPGASVKLSCKASGYTFTNQDINWVKQRPGQGLEWIGWIFPRDGRTHYNENFKGKATLTVDTSSSTAFMELHSLTAEDSAVYFCAIYGIALDHWGQGTSVTVSS

SEQ ID NO: 381 - protein, 116 aa, synthetic construct
  REGION 1..116: note = mice-40
QVQLQQSGPELVKPGASVKLSCKASGYTFTDYDVNWVKQRPGQGLEWIGWNYPRDGRTNYNEKFKGKATLTVDISSSTAYMELHSLTSEDSAVYFCARYGSALNVWGTGTSVTVSS

SEQ ID NO: 382 - protein, 116 aa, synthetic construct
  REGION 1..116: note = mice-41
QIQLVQSGPELVKPGASVKLSCKASGYTFTKYDINWVKQRPGQGLEWIGWIYPRDGSTKYNEKFKGKATLTVDTSSNTAYMELHSLTSEDSAVYFCARYSAAMDYWGQGTSVTVSS

SEQ ID NO: 383 - protein, 116 aa, synthetic construct
  REGION 1..116: note = mice-42
QVQLQQPGAELGKPGASVKLSCKASGYTFTNYDINWVKQRPGQGLEWIGWNYPRDGRTKYNEKFKGKATLTVDTSSSTAYMELHSLTSEDSAVYFCARYGYAMDYWGQGTSVTVSS

SEQ ID NO: 384 - protein, 116 aa, synthetic construct
  REGION 1..116: note = mice-43
QVQLQQPGAELGKPGASVKLSCKASGYTFTNYDINWMKLRPGQGLEWIGWNYPRDGRTKYNEKFKGKATFTVDTSSSTAYMELHSLTSEDSAVYFCARYGYAMDYWGQGTSVTVSS

SEQ ID NO: 385 - protein, 116 aa, synthetic construct
  REGION 1..116: note = mice-44
QIQLVQSGPELVKPGASVKLSCRASGYTFTSYDINWVKQRPGQGLEWIGWIFPRDGRTEYNAKFKGKATLTVDTSSNTAYMELHSLTSDDSAVYFCARYGSTLDVLGAGTTVTVSS

SEQ ID NO: 386 - protein, 116 aa, synthetic construct
  REGION 1..116: note = mice-45
EVQLQQSGPELVKPGASVKLSCKASGYTFTNQDINWVKQRPGQGLEWIGWIFPRDGRTHYNENFKDKATLTVDTSSSTAYMEVHSLTAEDSAVYFCAIYGIALDHWGQGTSVTVSS

SEQ ID NO: 387 - protein, 116 aa, synthetic construct
  REGION 1..116: note = mice-46
QIQLVQSGPELVKPGASVKLSCKASGYTFTRQDINWVKQRPGQGLEWIGWIYPRDGSTKYNEKFKGKVTLTVDTSSSTAYMELHSLTSEDSAVYFCATYGSSLDNWGQGTTLTVSS

SEQ ID NO: 388 - protein, 120 aa, synthetic construct
  REGION 1..120: note = mice-47
QVQLQQSGPELVKPGASVKIPCKTSGYTFTDYNINWVKRSHGKSLEWIGDINPDSGGTSYNQRFKDKATLTVDKSSSTAYMELRSLTSGDTAVYYCARWGYYGGRGFDYWGQGTTLTVSS

SEQ ID NO: 389 - protein, 116 aa, synthetic construct
  REGION 1..116: note = mice-48
QVQLQQSGAELGKPGASVKLSCKASGYTFTNYDINWMKQRPGQGLEWIGWNYPRDGRTKYNEKFKGKATLTVDTSSSTAYMELHSLTSEDSAVYFCARYGYAMDYWGQGTSVTVSS

SEQ ID NO: 390 - protein, 116 aa, synthetic construct
  REGION 1..116: note = mice-49
EVQLQQSGPDLVKPGASVKLSCKASGYTFASYDINWVKQRPGQGLEWIGWIYPRDGRTDYNEKFKGKATLTVDTSSSTAYMELHSLTSEDSAVYFCARYGYALGYWGQGTSVTVSS

SEQ ID NO: 391 - protein, 116 aa, synthetic construct
  REGION 1..116: note = mice-50
QVQLQQSGPELAKPGASVKLSCKASGYTFTSRDINWVKQRPGQGLEWIGWIYPRDGRTNYNEKFKDKATLTVDTSSSTAYMDLHSLTSEDSAVYFCATYGSSLDHWGQGTTLTVSS

SEQ ID NO: 392 - protein, 116 aa, synthetic construct
  REGION 1..116: note = mice-51
EVQLQQSGPELVKPGASVKLSCKASGYTFTNQDINWVKQRPGQGLEWIGWIFPRDGRTHYNENFKGKATLTVDTSSSTAYMELHSLTAEDSAVYFCAIYGAAMDHWGQGTSVTVSS

SEQ ID NO: 393 - protein, 116 aa, synthetic construct
  REGION 1..116: note = mice-52
QVQLQQSGPELVKPGTSVKLSCKASGYTFTDQDINWVKQRPGQGLEWIGWIFPRDGRTHYNEKFKGKATLTVDTSSSTAYMELHSLTSEDSAVYFCSIYGSAMDLWGQGTSVTVSS

SEQ ID NO: 394 - protein, 116 aa, synthetic construct
  REGION 1..116: note = mice-53
QVQLQQSGPELVKPGASVKLSCKASGYTFTNQDINWVKQRPGQGLEWIGWIFPRDGRTHNNENFKGKATLTVDTSSSTAYMELHSLTAEDSAVYFCAIYGFAMDHWGQGTSVTVSS

SEQ ID NO: 395 - protein, 116 aa, synthetic construct
  REGION 1..116: note = mice-54
EVQLQQSGPELVKPGASVKLSCKASGYTSTSYDINWVKQRPGQGLEWIGWIYPRDGRTNYNEKFKGKATLTVDTSSSTAFMELHSLTSEDSAVYFCARYSAAMDYWGQGTSVTVSS

SEQ ID NO: 396 - protein, 120 aa, synthetic construct
  REGION 1..120: note = mice-55
QVQLQQPGSELVRPGASVKIPCKTSGYSFTDFNINWVRQSHGKSLEWIGDINPDSGGTIYNQKFKGKATLTVDKSSSTAYMELRSLTSEDTAVYYCARWGYYGGEGFDYWGQGTTLTVSS

SEQ ID NO: 397 - protein, 116 aa, synthetic construct
  REGION 1..116: note = mice-56
QIQLVQSGPELVKPGASVKLSCKASGYTFTSSDINWVKQRPGQGPEWIGWIYPRDGRTHYNEKFKGKATLTVDTSSSTAYMELHSLTSEDSAVYFCARYAYALNSWGQGTSVTVSS

SEQ ID NO: 398 - protein, 116 aa, synthetic construct
  REGION 1..116: note = mice-57
QVQLQQPGPELVKPGASVRVSCKASGYTFTRQDINWVKQRAGQGLEWIGWIYPRDGRTEYNEKFKGKVTLTVDTSSSTAYMELHSLTSEDSAVYFCATYGSSLDNWGQGATLTVSS

SEQ ID NO: 399 - protein, 116 aa, synthetic construct
  REGION 1..116: note = mice-58
QVQLQQSGPELVKPGASVKLSCKASGYTFTSYDINWVKQRPGQGLEWIGWIFPRDGDTKYNEKFKGKATLTVDTSSSTAFMELHSLTSEDSAVYFCARYGSTLDVWGTGTTVTVSS

SEQ ID NO: 400 - protein, 116 aa, synthetic construct
  REGION 1..116: note = mice-59
QIQLVQSGPELVMPGASVKLSCQASGYTFTNSDINWVKQRPGQGLEWIGWVYPGDGSTYYNEKFKGKATLTVDTSSSTAYMDLYSLTSEDSAVYFCARYAYALNSWGQGTSVTVSS

SEQ ID NO: 401 - protein, 116 aa, synthetic construct
  REGION 1..116: note = mice-60
QIQLVQSGPELVKPGASVKLSCKASGYTFTNSDINWVKQRPGQGLEWIGWIYPRDGSTYYNEKFKGKATLTVDTSSTTAYMEFHSLTSEDSAVYFCARYAYALNSWGQGTSVTVSS

SEQ ID NO: 402 - protein, 116 aa, synthetic construct
  REGION 1..116: note = mice-61
QVQLQQSGPEVVKPGASVKLSCKASGYTFTSYDINWVKQRPGQGLEWIGWIFPRDGRTEYNEKFKDKATLTVDTSSSTAYMELHSLTSEDSAFYFCARYGSSLDYWGQGTTLTVSS

SEQ ID NO: 403 - protein, 116 aa, synthetic construct
  REGION 1..116: note = mice-62
QIQLVQSGPELVKPGASVKLSCQASGYTFTNSDINWVKQRPGQGLDWIGWVYPRDGSTYYNEKFKGKATLTVDTSSSTAYMELYSLTSEDSAVYFCARYAYALNYWGQGTSVTVSS

SEQ ID NO: 404 - protein, 116 aa, synthetic construct
  REGION 1..116: note = mice-63
QIQLVQSGPELVKPGASVKLSCKASGYTFTSIDINWVKQRPGQGLEWIGWIYPRDGRTDYNEKFKGKATLTVDTSSSTAYMELHSLTSEDSAVYFCARYTSSLDYWGQGTTLTVSS

SEQ ID NO: 405 - protein, 116 aa, synthetic construct
  REGION 1..116: note = mice-64
QVQLQQSGPELVKPGASVKLSCKASGYTFTSIDINWVKQRPGQGLEWIGWIYPRDGRTNYNEKFKGKATLTVDTSSSTAYMDLHSLTSEDSAVYFCARYTSSLDYWGQGTTLTVSS

SEQ ID NO: 406 - protein, 116 aa, synthetic construct
  REGION 1..116: note = mice-65
QVQLQQSGPELVKPGASVKLSCRASGYTFTSYDINWVKQRPGQGLEWIGWNYPRDGRTDYNEKFKGKATLTVDTSSNTAYMDLHSLTSEDSAVYFCTRYGSTLDVWGTGTSVTVSS

SEQ ID NO: 407 - protein, 116 aa, synthetic construct
  REGION 1..116: note = mice-66
QIQLVQSGPELVKPGASVKLSCQASGYTFTNSDINWVKQRPGQGLEWIGWVYPRDGSAYYNEKFKGKATLTVDTSSSTAYMELYSLTSEDSAVYFCARYAYALNFWGQGTSVTVSS

SEQ ID NO: 408 - protein, 116 aa, synthetic construct
  REGION 1..116: note = mice-67
QVQLKQSGPELVKPGASVKVSCKASGYTFTRQDINWVKQRPGQGLEWIGWIYPRDGRTEYNEKFKGKVTLTVDTSSSTAYMELHSLTSEDSAVYFCATYGSSLDNWGQGTTLTVSS

SEQ ID NO: 409 - protein, 116 aa, synthetic construct
  REGION 1..116: note = mice-68
QVQLQQPGPELVKPGASVKLSCKASGYTFTNQDINWVKQRPGQGLEWIGWIFPRDGRTHYNEKFKGKATLTVDTSSSTAYMELHSLTAEDSAVYFCAIYGSAMDQWGQGTSVTVSS

SEQ ID NO: 410 - protein, 116 aa, synthetic construct
  REGION 1..116: note = mice-69
EVQLQQSGPELVKPGASVKLSCKASGYTFTNSDINWVKQRPGQGLEWIGWIYPRDGSTYYNEKFKGKATLTVDTSSTTAYMEFHSLTSEDSAVYFCARYAYALNSWGQGTSVTVSS

SEQ ID NO: 411 - protein, 121 aa, synthetic construct
  REGION 1..121: note = mice-70
QVQLQQPGAELMKPGASVKLSCKATGYTFTAYWLEWVKQRPGHGLEWIGEILPGSGYAYYNAKFKGKATFTADISSNTAYMQLSGLTTEDSAIYYCARRGSNYPYWYFDVWGTGTTVTVSS

SEQ ID NO: 412 - protein, 121 aa, synthetic construct
  REGION 1..121: note = mice-71
QVQLQQSGAELMKPGASVKLSCKATGYTFTGYWLEWVKQRPGHGLEWIGETLPGSGYNKYNEKFKGKATFTADTSSNTAHMQLSSLTTEDSAIYYCARRGSNYPYWYFDVWGTGTTVTVSS

SEQ ID NO: 413 - protein, 121 aa, synthetic construct
  REGION 1..121: note = mice-72
QVQLQQSGAELMKPGASVKLSCKATGYTFTGYWLEWVKQRPGHGLEWIGETLPGSGYNKYNEKFKGKATFTADTSSNTAHMQLSSLTTEDSAIYYCARRGSNYPYWYFDVWGTGTSVTVSS

SEQ ID NO: 414 - protein, 116 aa, synthetic construct
  REGION 1..116: note = mice-73
QVQLQQSGPELVKPGASVKLSCRASGYTFTSYDINWVKQRPGQGLEWIGWNYPRDGRTDYNEKFKGKATLTVDTSSNTAYMELHSLTSEDSAVYFCTRYGSTLDVWGTGTTVTVSS

SEQ ID NO: 415 - protein, 116 aa, synthetic construct
  REGION 1..116: note = mice-74
QIQLVQSGPELVKPGASVKLSCRASGYTFTSYDINWVKQRPGQGLEWIGWNYPRDGRTDYNEKFKGKATLTVDTSSNTAYMELHSLTSEDSAVYFCTRYGSTLDVWGTGTTVTVSS

SEQ ID NO: 416 - protein, 116 aa, synthetic construct
  REGION 1..116: note = mice-75
QVQLQQPGPELVKPGASVKLSCRASGYTFTSYDINWVKQRPGQGLEWIGWIFPRDGRTEYNAKFKGKATLTVDTSSNTAYMELHSLTSDDSAVYFCARYGSTLDVLGAGTTVTVSS

SEQ ID NO: 417 - protein, 116 aa, synthetic construct
  REGION 1..116: note = mice-76
EVQLQQSGPELVKPGASVKLSCRASGYTFTSYDINWVKQRPGQGLEWIGWIFPRDGRTEYNEKFKGKATLTVDTSSNTAYMELHSLTSEDSAVYFCARYGSTLDVWGTGTTVTVSS

SEQ ID NO: 418 - protein, 116 aa, synthetic construct
  REGION 1..116: note = mice-77
QIQLVQSGPELVKPGASVKLSCRASGYTFTSYDINWVKQRPGQGLEWIGWIFPRDGRTEYNEKFKGKVTLTVDTSSNTAYMELHSLTSEDSAVYFCARYGSTLDVWGPGTTVTVSS

SEQ ID NO: 419 - protein, 116 aa, synthetic construct
  REGION 1..116: note = mice-78
QVQLQQFGPELVKPGSSVKLSCKASGYTFTDYDVNWVKQRPGQGLEWIGWNYPRDGRTNYNEKFKGKATLTVDTSSSTAYMELHSLTSEDSAVYFCARYGSALNVWGTGTTVTVSS

SEQ ID NO: 420 - protein, 116 aa, synthetic construct
  REGION 1..116: note = mice-79
QVQLQQSGPELVKPGASVKLSCKASGYTFTDYDVNWVKQRPGQGLEWIGWNYPRDGRTNYNEKFKGKATLTVDISSSTAYMELHSLTSEDSAVYFCARYGSALNVWGTGTTVTVSS

SEQ ID NO: 421 - protein, 116 aa, synthetic construct
  REGION 1..116: note = mice-80
QIQLVQSGPELVKPGASVKLSCQASGYTFTNSDINWVKQRPGQGLEWIGWVYPRDGSTYYNEKFKGKATLTVDTSSSTAYMELYSLTSEDSAVYFCARYAYALNSWGQGTSVTVSS

SEQ ID NO: 422 - protein, 116 aa, synthetic construct
  REGION 1..116: note = mice-81
QIQLVQSGPELVKPGASVKLSCQASGYTFTNSDINWVKQRPGQGLEWIGWVYPRDGSTYYNEKFKAKATLTVDTSSSTAYMELYSLTSEDSAVYFCARYAYALNYWGQGTSVTVSS

SEQ ID NO: 423 - protein, 116 aa, synthetic construct
  REGION 1..116: note = mice-82
QIQLVQSGPELVKPGASVKLSCQASGYTFTNSDINWVKQRPGQGLEWIGWVYPRDGRTYYNEKFKGKATLTVDTSSSTAYMELYSLTSEDSAVYFCARYAYALNSWGQGTSVTVSS

SEQ ID NO: 424 - protein, 116 aa, synthetic construct
  REGION 1..116: note = mice-83
QIQLVQSGPELVKPGASVKLSCKASGYTFTNSDINWVKQRPGQGLEWIGWVYPRDGSTYYNEKFKGKATLTVDTSSSTAYMELHSLTSEDSAVYFCTRYAYALNSWGQGTSVTVSS

SEQ ID NO: 425 - protein, 116 aa, synthetic construct
  REGION 1..116: note = mice-84
QIQLVQSGPELVKPGASVKLSCKASGYTFTNSDINWVKQRPGQGLEWIGWIYPRDGSTYYNEKFKGKATLTVDTSSTTAYMEFHSLTSEDSAVYFCARYAYTLNSWGQGTSVTVSS

SEQ ID NO: 426 - protein, 116 aa, synthetic construct
  REGION 1..116: note = mice-85
EVQLQQSGAELARPGASVKMSCKASGYTFTNSDINWVKQRPGQGLEWIGWIYPRDGSTYYNEKFKGKATLTVDTSSSTAYMELHSLTSEDSAVYFCTRYAYALNSWGQGTSVTVSS

SEQ ID NO: 427 - protein, 116 aa, synthetic construct
  REGION 1..116: note = mice-86
QVQLQQSGPELVKPGASVKLSCKASGYTFTNSDINWVKQRPGQGLEWIGWIYPRDGSTYYNEKFKGKATLTVDTSSSTAYMELHSLTSEDSAVYFCTRYAYALNSWGQGTSVTVSS

SEQ ID NO: 428 - protein, 116 aa, synthetic construct
  REGION 1..116: note = mice-87
EVQLQQSGPELVKPGASVKLSCQASGYTFTNSDINWVKQRPGQGLEWIGWVYPRDGRTYYNEKFKGKATLTVDTSSSTAYMELYSLTSEDSAVYFCARYAYALNSWGQGTSVTVSS

SEQ ID NO: 429 - protein, 116 aa, synthetic construct
  REGION 1..116: note = mice-88
EVQLQQSGPELVRPGASVKLSCKASGYSFTSGDINWVKQRPGQGLEWIAWIYPRDGSTEYNEKFKGKATLTVDTSSSTAYMELHSLTSEDSAVYFCARYDYAFVYWGQGTLVTVSA

SEQ ID NO: 430 - protein, 116 aa, synthetic construct
  REGION 1..116: note = mice-89
EVQLQQSGPELVRPGTSVRLSCKTSGYTFTSRDINWVKQRPGQGLEWIAWIYPRDGSTEYNEKFKGKATLTVDTSSSTAYMELHSLTSEDSAVYFCTRYDYAFTYWGQGTLVTVSA

SEQ ID NO: 431 - protein, 116 aa, synthetic construct
  REGION 1..116: note = mice-90
EVQLQQSGPEVVKPGASVKLSCKASGYTFTRNDINWVKQRPGQGLEWIGWIYPRDGSSKNNEKFKGKATLTVDTSSSTAYMELHSLTSEDSAVYFCARYDYAFVYWGQGTTVTVSS

SEQ ID NO: 432 - protein, 116 aa, synthetic construct
  REGION 1..116: note = mice-91
EVQLQQSGPELVKPGASVKLSCKASGYTFTSYDINWVKQRPGQGLEWIGWIYPRDGRTKYSEKFKGKATLTVDTSSSTAYMELHSLTSEDSAVYFCARYGASMDYWGQGTSVTVSS

SEQ ID NO: 433 - protein, 116 aa, synthetic construct
  REGION 1..116: note = mice-92
EVQLQQSGPELVKPGASVKLSCKASGYTFTSYDINWVKQSPGQGPEWIGWVYPRDGRTQYNEKFKGKATLTVDTSSSTAYMELHSLTSEDSAVYFCARYGASMDYWGQGTSVTVSS

SEQ ID NO: 434 - protein, 116 aa, synthetic construct
  REGION 1..116: note = mice-93
EVQLQQSGPELAKPGASVKLSCKASGYTFTSYDINWVKQRPGQGLEWIGWIYPRDGNTNYNEKFKGKATLTVDTSSNTASMELHSLTSEDSAVYFCARYSAAMDYWGQGTSVTVSS

SEQ ID NO: 435 - protein, 116 aa, synthetic construct
  REGION 1..116: note = mice-94
QVQLQQPGPELVKPGASVKLSCKASGYTSTSYDINWVKQRPGQGLEWIGWIYPRDGRTNYNEKFKGKATLTVDTSSSTAFMELHSLTSEDSAVYFCARYSAAMDYWGQGTSVTVSS

SEQ ID NO: 436 - protein, 116 aa, synthetic construct
  REGION 1..116: note = mice-95
QVQLQQSGPELVKPGASVKLSCKASGYTFTSYDINWVKQRPGQGLEWIGWIFPRDGRTNYNEKFKDKATLTVDTSSSTAFMELHRLTSEDSAVYFCARYSAAMDYWGQGTSVTVSS

SEQ ID NO: 437 - protein, 116 aa, synthetic construct
  REGION 1..116: note = mice-96
EVQLQQSGAELGKPGASVKLSCKASGYTFTNYDINWMKQRPGQGLEWIGWNYPRDGRTKYNEKFKGKATLTVDTSSSTAYMELHSLTSEDSAVYFCARYGYAMDYWGQGTSVTVSS

SEQ ID NO: 438 - protein, 116 aa, synthetic construct
  REGION 1..116: note = mice-97
QVQLQQSGPDLVKPGASVKLSCKASGYIFASYDLNWVKQRPGQGLEWIGWIYPRDGRTDYNEKFKGKATLTVDTSSSTAYMELHSLTSEDSAVYFCARYGYALDYWGQGTSVTVSS

SEQ ID NO: 439 - protein, 116 aa, synthetic construct
  REGION 1..116: note = mice-98
QIQLVQSGPELVKPGASVKVSCKASGYTFTRQDINWVKQRPGQGLEWIGWIYPRDGRTEYNEKFKGKATLTVDTSSSTAFMELHSLTSEDSAVYFCARYSAAMDYWGQGTSVTVSS

SEQ ID NO: 440 - protein, 116 aa, synthetic construct
  REGION 1..116: note = mice-99
QIQLVQSGPELVKPGASVKLSCKASGYTFTNSDINWVKQRPGQGLEWIGWNYPRDGRTKYNEKFKGKATFTVDTSSSTAYMDLQSLTSEDSAVYFCTRYGSAMDYWGQGTSVTVSS

SEQ ID NO: 441 - protein, 116 aa, synthetic construct
  REGION 1..116: note = mice-100
EVQLQQSGPELVKPGASVKLSCKASGYTFTNQDINWVKQRPGQGLEWIGWIFPRDGRTHYNENFKGKATLTVDTSSSTAYMELHSLTAEDSAVYFCAIYGIALDHWGQGTSVTVSS

SEQ ID NO: 442 - protein, 116 aa, synthetic construct
  REGION 1..116: note = mice-101
QVQLQQSGPELVKPGASVKLSCKASGYTFTNQDINWVKQRPGQGLEWIGWIFPRDGRTHYNENFKGKATLTVDTSSNTAYMELHSLTAEDSAVYFCAIYGIALDHWGQGTSVTVSS

SEQ ID NO: 443 - protein, 116 aa, synthetic construct
  REGION 1..116: note = mice-102
QVQLQQSGPELVKPGASVKLSCKASGYTFTNQDINWVKQRPGQGLEWIGWIFPRDGRTHYNENFKGKATLTVDTSSSTAYMELHSLTAEDSAVYFCAIYGIALDHWGQGTSVTVSS

SEQ ID NO: 444 - protein, 116 aa, synthetic construct
  REGION 1..116: note = mice-103
QVQLQQSGPELVKPGASVKLSCKASGYTFTNQDINWVKQRPGQGLEWIGWIFPRDGRTHYNENFKGKATLTVDTSSSTAYMELHSLTAEDSAVYFCAIYGAAMDHWGQGTSVTVSS

SEQ ID NO: 445 - protein, 116 aa, synthetic construct
  REGION 1..116: note = mice-104
QVQLQQSGPELVKPGTSVKLSCKASGYTFTNQDINWVKQRPGQGLEWIGWIFPRDGRTHYNENFKGKATLTVDTSSSTAYMELHSLTAEDSAVYFCSIYGIAMDHWGQGTSVTVSS

SEQ ID NO: 446 - protein, 116 aa, synthetic construct
  REGION 1..116: note = mice-105
QVQLQQPGPELVKPGASVKLSCKASGYTFTNQDINWVKQRPGQGLEWIGWIFPRDGRTHNNENFKGKATLTVDTSSSTAYMELHSLTAEDSAVYFCAIYGFAMDHWGQGTSVTVSS

SEQ ID NO: 447 - protein, 116 aa, synthetic construct
  REGION 1..116: note = mice-106
QVQLQQSGPELVKPGASVKLSCKASGYTFTNQDINWVKQRPGQGLEWIGWIFPRDGRTHYNENFKGKATLTVDTSSSTAYMELHSLTAEDSAVYFCAIYGFAMDHWGQGTSVTVSS

SEQ ID NO: 448 - protein, 116 aa, synthetic construct
  REGION 1..116: note = mice-107
QVQLQQPGPELVKPGASVKLSCKASGYTFTNQDINWVKQRPGQGLEWIGWIFPRDGRTHYNENFKGKATLTVDTSSSTAYMELHSLTAEDSAVYFCAIYGAAMDDWGQGTSVTVSS

SEQ ID NO: 449 - protein, 116 aa, synthetic construct
  REGION 1..116: note = mice-108
QVQLQQPGPELVKPGASVKLSCKASGYTFTNQDINWVKQRPGQGLEWIGWIFPRDGRTHYNENFKGKATLTVDTSSSTAFMELHSLTAEDSAVYFCAIYGIALDHWGQGTSVTVSS

SEQ ID NO: 450 - protein, 116 aa, synthetic construct
  REGION 1..116: note = mice-109
EVQLQQSGPELVKPGASVKLSCKASGYTFTNQDINWVKQRPGQGLEWIGWIFPRDGRTHYNENFKGKATLTVDTSSSTAYMELHSLTAEDSAVYFCAIYGFAMDHWGQGTSVTVSS

SEQ ID NO: 451 - protein, 116 aa, synthetic construct
  REGION 1..116: note = mice-110
EVQLQQSGPELVKPGASVKLSCKASGYTFTNQDINWVKQRPGQGLEWIGWNFPRDGRTHYNENFKGKATLTVDTSSSTAYMELHSLTAEDSAVFFCAIYGFALDHWGQGTSVTVSS

SEQ ID NO: 452 - protein, 116 aa, synthetic construct
  REGION 1..116: note = mice-111
QVQLQQSGPELVKPGASVKLSCKASGYTFTNQDINWVKQRPGQGLEWIGWIFPRDGRTHYNENFKGKATLTVDTSSSTAYMELHSLTAEDSAVYFCALYGFAMDQWGQGTSVTVSS

SEQ ID NO: 453 - protein, 116 aa, synthetic construct
  REGION 1..116: note = mice-112
EVQLQQSGPELVKPGASVKLSCKASGYTFTNQDINWVKQRPGQGLEWIGWIFPRDGRTHYNEKFKGKATLTVDTSSSTAYMELHSLTAEDSAVYFCAIYGSAMDQWGQGTSVTVSS

SEQ ID NO: 454 - protein, 120 aa, synthetic construct
  REGION 1..120: note = mice-113
EVQLQQSGPELVKPGTSVKIPCKTSGYTFTDYNINWVKQSHGKSLEWIGDINPDSGGTVYNQRFKDKATLTVDKSSSTAYMELRSLTSGDTAVYYCARWGYYDGRGFDYWGQGTTLTVSS

SEQ ID NO: 455 - protein, 117 aa, synthetic construct
  REGION 1..117: note = mice-114
EVQLQQSGAELVRPGASVTLSCKASGYTFTDYEIHWVKQIPVHGLEWIGVFDPETGGAAYNQKFKGKAILTADKSSSTAYMELRSLTSEDSAVYYCTTPWLNYFEYWGQGTTLTVSS

SEQ ID NO: 456 - protein, 115 aa, synthetic construct
  REGION 1..115: note = mice-115
QVQLQQSVAELVRPGASVKLSCTASGFNIEDTYMHWVKQRPEQGLEWIGRVDPASGNTKYGPKFQGKATITADTSSNTAYLQLSSLSSADTAIYYCSTWGLFDYWGQGTTLTVSS

SEQ ID NO: 457 - protein, 116 aa, synthetic construct
  REGION 1..116: note = mice-116
QVQLQQPGPELVKPGASVKLSCKASGYTFTSIDINWVKQRPGQGLEWIGWIYPRDGRTHYNEKFKGKATLTVDTSSSTAYMELHSLTSEDSAVYFCARYTSSLDYWGQGTTLTVSS

SEQ ID NO: 458 - protein, 116 aa, synthetic construct
  REGION 1..116: note = mice-117
QVQLQQSGPELVKPGASVKLSCKASGYTFTSIDINWVKQRPGQGLEWIGWIYPRDGRTDYNEKFKGKATLTVDTSSSTAYMELHSLTSEDSAVYFCARYGSSLDYWGQGTTLTVSS

SEQ ID NO: 459 - protein, 115 aa, synthetic construct
  REGION 1..115: note = mice-118
QVQLQQSGPELVKPGTSVRLSCKASGYTFTNSDINWVKQRPGQGLEWIGWIYPRDGRTDYNEKFKGKATLTVDTSSSTAYMELHSLTSEDSAVYFCARYGSSLYWGQGTTLTVSS

SEQ ID NO: 460 - protein, 116 aa, synthetic construct
  REGION 1..116: note = mice-119
EVQLQQSGPELVKPGASVKLSCKASGYTFTSSDINWVKQRPGQGPEWIGWIYPRDGRTHYNEKFKGKATLTVDTSSSTAYMELHSLTSEDSAVYFCAPYGSSLAYWGQGTTLTVSS

SEQ ID NO: 461 - protein, 116 aa, synthetic construct
  REGION 1..116: note = mice-120
QIQLVQSGPELVKPGASVKLSCKASGYTFTSSDINWVKQRPGQGLEWIGWIYPRDGSTHYNENFKGKATLTVDTSSSTAYMELHSLTSEDSAVYFCAPYGSSLAYWGQGTTLTVSS

SEQ ID NO: 462 - protein, 116 aa, synthetic construct
  REGION 1..116: note = mice-121
QVQLKESGPEVVKPGASVKLSCKASGYTFTSYDINWVKQRPGQGLEWIGWIYPRDGRTEYNEKFKDKATLTVDTSSSTAYMELHSLTSEDSAVYFCARYGSSLDYWGQGTTLTVSS

SEQ ID NO: 463 - protein, 116 aa, synthetic construct
  REGION 1..116: note = mice-122
QIQLVQSGPELVKPGASVKVSCKASGYTFTRQDINWVKQRPGQGLEWIGWIYPRDGSTKYNEKFKGKVTLTVDTSSSTAYMELHSLTSEDSAVYFCATYGSSLDNWGQGTTLTVSS

SEQ ID NO: 464 - protein, 116 aa, synthetic construct
  REGION 1..116: note = mice-123
QVQLQQSGPELVKPGASVKVSCKASGYTFTRQDINWVKQRPGQGLEWIGWIYPRDGSTKYNEKFKGKVTLTVDTSSSTAYMELHSLTSEDSAVYFCATYGSSLDNWGQGTTLTVSS

SEQ ID NO: 465 - protein, 116 aa, synthetic construct
  REGION 1..116: note = mice-124
QIQLVQSGPELVKPGASVKVSCKASGYTFTRQDINWVKQRPGQGLEWIGWIYPRDGRTEYNEKFKGKVTLTVDTSSSTAYMELHSLTPEDSAVYFCATYGSSLDNWGQGTTLTVSS

SEQ ID NO: 466 - protein, 116 aa, synthetic construct
  REGION 1..116: note = mice-125
QVQLKESGPELVKPGASVKVSCKASGYTFTRQDINWVKQRPGQGLEWIGWIYPRDGRTEYNEKFKGKVTLTVDTSSSTAYMELHSLTSEDSAVYFCATYGSSLDNWGQGTTLTVSS

SEQ ID NO: 467 - protein, 116 aa, synthetic construct
  REGION 1..116: note = mice-126
QVQLQQSGAELAKPGASVKLSCKASGYTFTRQDINWVKQRPGQGLEWIGWIYPRDGRTEYNEKFKGKVTLTVDTSSSTAYMELHSLTSEDSAVYFCATYGSSLDNWGQGTTLTVSS

SEQ ID NO: 468 - protein, 116 aa, synthetic construct
  REGION 1..116: note = mice-127
QVQLQQPGPELVKPGASVKVSCKASGYTFTRQDINWVKQRPGQGLEWIGWIYPRDGRTEYNEKFKGKVTLTVDTSSNTAYMELHSLTSEDSAVYFCATYGSSLDNWGQGTTLTVSS

SEQ ID NO: 469 - protein, 116 aa, synthetic construct
  REGION 1..116: note = mice-128
QVQLQQSGPELVKPGASVKVSCKASGYTFTRQDINWVKQRPGQGLEWIGWIYPRDGRTEYNEKFKGKVTLTVDTSSSTAYMELHSLTSEDSAVYFCATYGSSLDNWGQGTTLTVSS

SEQ ID NO: 470 - protein, 116 aa, synthetic construct
  REGION 1..116: note = mice-129
EVQLQQSGPELVKPGASVKVSCKASGYTFTRQDINWAKQRPGQGLEWIGWIYPRDGRTEYNEKFKGKVTLTVDTSSSTAYMELHSLTPEDSAVYFCATYGSSLDNWGQGTTLTVSS

SEQ ID NO: 471 - protein, 116 aa, synthetic construct
  REGION 1..116: note = mice-130
EVQLQQSGPELVKPGASVKVSCKASGYTFTRQDINWVKQRPGQGLEWIGWIYPRDGRTEYNEKFKGKVTLTVDTSSSTAYMELHSLTSEDSAVYFCATYGSSLDNWGQGTTLTVSS

SEQ ID NO: 472 - protein, 114 aa, synthetic construct
  REGION 1..114: note = mice-131
EVQLQQSVAELVRPGASVKLSCTASGFNIENTYIHWVKQRPEQGLEWIGRIDPASGNTKYAPKFQGKATITADTSSNTAYLQLSSLTSEDTAIYYCASTGPDYWGQGTTLTVSS

SEQ ID NO: 473 - protein, 119 aa, synthetic construct
  REGION 1..119: note = mice-132
QIQLVQSGPELKKPGETVEISCKASGYTFTTHGMNWVKQAPGKGLKWMGWINTFSGVPTYADDFRGRFDFFLATSASTAYLQINNLKNEDTATYFCARDYGTAYAMDSWGQGTSVTVSS

SEQ ID NO: 474 - protein, 119 aa, synthetic construct; identical to SEQ ID NO: 202
  REGION 1..119: note = mice-133

SEQ ID NO: 475 - protein, 119 aa, synthetic construct
  REGION 1..119: note = mice-134
EVQLQQSGPELVKPGASVKISCKASGYTFTDYYMNWVKQSHGKSLEWIGDVNPNSGGTSQNQKFKGKATLTVDKSSTTAYMELRSLTSEDSAVYHCARGSNYVGGFTYWGQGTLVAVSA

SEQ ID NO: 476 - protein, 122 aa, synthetic construct
  REGION 1..122: note = mice-135
QVQLQQPGAELVKPGASVKLSCKASGYTFSNDWMHWVKQRPGRGLEWIGRIDPDSGGTKYNEKFKNKATLTVDKPSSTVYMQLSSLTSEDSAVYYCARSEGYYGSRSYFDYWGQGTTLTVSS

SEQ ID NO: 477 - protein, 119 aa, synthetic construct; identical to SEQ ID NO: 297
  REGION 1..119: note = mice-136

SEQ ID NO: 478 - protein, 107 aa, synthetic construct; identical to SEQ ID NO: 102
  REGION 1..107: note = mice-137

SEQ ID NO: 479 - protein, 111 aa, synthetic construct
  REGION 1..111: note = mice-138
DIVLTQSPASLPVSLGQRATISCRASKSVSTSSYSYMHWYQQKPGQPPKLLIKYASNLESGVPARFSGSGSGTDFTLNIHPVEEEDAATYYCQHSKEFPWTFGGGTKLEIK

SEQ ID NO: 480 - protein, 107 aa, synthetic construct
  REGION 1..107: note = mice-139
DIKMTQSPSSMYASLGERVTITCKASQDINSFLSWFQQKPGKSPKTLIYRANRLVDGVPSRFSGSGSGQDYSLTISSLEYEDMGIYYCLQCDEFPYTFGGGTKLEIK

SEQ ID NO: 481 - protein, 107 aa, synthetic construct
  REGION 1..107: note = mice-140
DIKMTQSPSSMYASLGERVTITCKASQDINSYLSWFQQKPGKSPKTLIYRANRLVDGVPSRFSASGSGQDYSLTISSLEYEDMGIYYCLQCDEFPYTFGGGTKLEIK

SEQ ID NO: 482 - protein, 107 aa, synthetic construct
  REGION 1..107: note = mice-141
DIVMTQSHKFMSTSVGDRVSITCKASQDVSTAVAWYQQKPGQSPKLLIYWASARHTGVPARFTGSGSGTDYTLTISSVQAEDLALYYCQQHYGTPPTFGGGTKLEIK

SEQ ID NO: 483 - protein, 106 aa, synthetic construct
  REGION 1..106: note = mice-142
QIVLTQSPAIMSVSPGEKVTITCSATSRVTYMHWFQQKPGTAPKLWIYSTSNVASGVPARFSGSGSVTSYSLTISRMEAADAATYYCQQRSSYPPTFGGGTKLEIK

SEQ ID NO: 484 - protein, 111 aa, synthetic construct
  REGION 1..111: note = mice-143
DIMLTQSPASLTVSLGQRATISCRASKSVSTSSYTYMHWYQQKPGQPPKLLIKYASNLESGVPARFSGSGSGTDFILNIHPVEEEDAATYYCQHSREFPWTFGGGTKLEIK

SEQ ID NO: 485 - protein, 111 aa, synthetic construct
  REGION 1..111: note = mice-144
DIVLTQSPASLAVSLGQRATISCRASKSVSTSSYNYMHWYQQKPGQPPKLLIKYASNLESGVPARFSGSGSGTDFTLNIHPVEEEDAATYYCQHSREFPWTFGGGTKLEIK

SEQ ID NO: 486 - protein, 108 aa, synthetic construct
  REGION 1..108: note = mice-145
QIVLTQSPALMSASPGEKVTMTCSASSSVSYMYWYQQKPRSSPKPWIYLTSNLASGVPARFSGSGSGTSYSLTISSMEAEDAATYYCQQWIIYPPMLTFGAGTKLELK

SEQ ID NO: 487 - protein, 110 aa, synthetic construct
  REGION 1..110: note = mice-146
DIVLTQSPASLAVSLGQRATISCKASQSVSFAGISLMHWYQQKPGQQPKLLIYRASNLEAGVPTRFSGSGSRTDFTLTINPVETDDVATYYCQQSNKDPTFGGGTKLELK

SEQ ID NO: 488 - protein, 108 aa, synthetic construct
  REGION 1..108: note = mice-147
DIVMTQSHKFMSTSVGDRVSITCKASQDVSTAVAWYQQKPGQSPKLLIYSASYRYTGVPDRFTGSGSGTDFTFTISSVQAEDLAVYYCHQHYSTPYTFGGGTKLEIKR

SEQ ID NO: 489 - protein, 113 aa, synthetic construct
  REGION 1..113: note = mice-148
DVVMTQTPLSLPVSLGDQASISCRSSQNIVHSNGDTYLDWFLQKPGQSPKLLIYKVSNRFSGVPDRFSGSGSGTDFTLKITRVESEDLGVYYCFQGSHDPYTFGGGTKLEIKR

SEQ ID NO: 490 - protein, 106 aa, synthetic construct
  REGION 1..106: note = mice-149
ENVLTQSPAIMSASPGERVTMTCSASSSVSYMHWYQQKSSTSPKLWISDTSKLASGVPGRFSGSGSGNSYSLTISSMEAEDVATYYCFQESGYPFTFGAGTKLELK

SEQ ID NO: 491 - protein, 113 aa, synthetic construct
  REGION 1..113: note = mice-150
DVVMTQTPLSLPVSLGDQASISCRSSQSIVHSNGDTYLEWYLQKPGQSPKLLIYKVSNRFSGVPDRFSGSGSGTDFTLKISRVEAEDLGVYYCFQGSHVPYTFGGGTKLEIKR

SEQ ID NO: 492 - protein, 106 aa, synthetic construct
  REGION 1..106: note = mice-151
ENVLTQSPAIMSASPGEKVTMTCSASSSVSYMHWYQQKSSTSPKLWIYDTSKLASGVPGRFSGSGSGNSYSLTISSMEAEDVATYYCFQESGYPLTFGAGTKLELK

SEQ ID NO: 493 - protein, 106 aa, synthetic construct
  REGION 1..106: note = mice-152
EILLTQSPAIIVASPGEKVTITCSATSSVIYMNWYQQKPGSSPKIWIYGISNLASGVPARFSGSGSGTSFSFTINSMEAEDVATYYCQQRSSYPFTFGSGTKLEIK

SEQ ID NO: 494 - protein, 107 aa, synthetic construct
  REGION 1..107: note = mice-153
DVQITQSPSYLAASPGETITMNCRASKSIRKYLAWYQEKPGKSNKILIYSGSTLQSGIPSRFSGSGSGTDFTLTISSLEPEDFAMYYCQQHNEYPLTFGAGTKLELK

SEQ ID NO: 495 - protein, 106 aa, synthetic construct
  REGION 1..106: note = mice-154
QIVLTQSPAIMSASPGEKVTMTCSASSSVSYMHWYQQKSGTSLKRWIYDTSKLASGVPARFSGSGSGTSYSLTVSSMEAEDAATYYCQQWTSTPYTFGGGTKLEIK

SEQ ID NO: 496 - protein, 106 aa, synthetic construct
  REGION 1..106: note = mice-155
QIVLTQSPAIMSASPGEKVTMTCSASSSVSYMHWYQQKSGTSPKRWIYDTSKLASGVPARFSGSGSGTSYSLTISTMEAEDAATYYCQQWTSTPYTFGGGTKLEIK

SEQ ID NO: 497 - protein, 107 aa, synthetic construct
  REGION 1..107: note = mice-156
DIQMTQTTSSLSASLGDRVTISCRASQDITNYLNWYQQKPDGTVKLLIYSTSRLHSGVPSRFSGSGSGTDYSLTISNLEQEDIATYFCQQGNTLPLTFGAGTKLELK

SEQ ID NO: 498 - protein, 107 aa, synthetic construct
  REGION 1..107: note = mice-157
DIKMTQSPSSMYASLGERVTITCKASQDINSYLSWFQQKPGKSPKTLIYRANRLLDGVPSRFSGSGSGQDYSLTISSLEYEDMGIYYCLQYDEFPYTFGGGTKLEIK

SEQ ID NO: 499 - protein, 107 aa, synthetic construct
  REGION 1..107: note = mice-158
DIVMTQPQKFMPTSVGDRVSVTCKASQNVGTNVAWYQQKAGQSPKVLIYSASYRYSGVPDRFTGSGSGTAFTLTISNVQSEDLAEYFCHQYNNFPFSFGSGTMLEIK

SEQ ID NO: 500 - protein, 107 aa, synthetic construct
  REGION 1..107: note = mice-159
DIKMTQSPSSMYASLGDRVTITCKASQDIDSYLSWFQQKPGKSPKTLMYHTNRLIVGVPSRFSGTGSGQDYALTISSLEYEDLGIYYCQQYDEFPSTFGSGTKLEIK

SEQ ID NO: 501 - protein, 107 aa, synthetic construct
  REGION 1..107: note = mice-160
DIQMTQTTSSLSASLGDRVTISCRASQDISNYLNWYQQKPDGTVKLLIYYTSRLHSGVPSRFSGSGSGTDYSLTISNLDQEDIATYFCQQGNTLPLTFGAGTKLELK

SEQ ID NO: 502 - protein, 107 aa, synthetic construct
  REGION 1..107: note = mice-161
DNVMTQSQKFMSTSVGDRVSVTCKASQNVGTNVAWYQQKAGQSPKVLIYSASYRYSGVPDRFTGSGSGTAFTLTISNVQSEDLAEYFCQQYNSFPFTFGSGTKLEIK

SEQ ID NO: 503 - protein, 112 aa, synthetic construct
  REGION 1..112: note = mice-162
DVVMTQTPLTLSVTIGQPASISCKSSQSLLDSDGKTYLSWLLQRPGQSPKRLIILVSKLDSGVPDRFTGSGSGTDFTLKISRVEAEDLGVYYCWQGTHFPQTFGGGTKLEIK

SEQ ID NO: 504 - protein, 112 aa, synthetic construct
  REGION 1..112: note = mice-163
DVLMTQAPLSLPVSLGDQASISCRSSQSIVHSNGDTYLDWYLQKPGQSLKLLIYKLSNRFSGVPDRFSGSGSGTDFTLKISRVEAEDLGVYHCFQGSHVPYTFGGGTKLEIK

SEQ ID NO: 505 - protein, 113 aa, synthetic construct
  REGION 1..113: note = mice-164
DVVMTQTPLSLPVSLGDQASISCRSSQSIVHGNGNTYLEWYLQKPGQSPKLLIYKVSNRFSGVPDRFSGSGSGTDFTLKISRVEAEDLGVYYCFQGSHVPYTFGGGTKLELKR

SEQ ID NO: 506 - protein, 113 aa, synthetic construct
  REGION 1..113: note = mice-165
DVVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLELKR

SEQ ID NO: 507 - protein, 112 aa, synthetic construct
  REGION 1..112: note = mice-166
DVVMTQTPLSLPVSLGDQASISCRSSQSIVHSNGSTYLEWYLQKPGQSPKLLIYKVSNRFSGVPDRFSGSGSGTDFTLKISRVEAEDLGVYYCFQGSHVPYTFGGGTKLEIK

SEQ ID NO: 508 - protein, 112 aa, synthetic construct
  REGION 1..112: note = mice-167
DVVMTQTPLSLPVSLGDQASISCRSSQNIVHSNGDTYLDWFLQKPGQSPQLLIYKVSNRFSGVPDRFSGSGSGTDFTLKISRVEAEDLGIYYCFQGSHVPYTFGGGTKLEIK

SEQ ID NO: 509 - protein, 112 aa, synthetic construct
  REGION 1..112: note = mice-168
DVVMTQTPLSLPVSLGDQASISCRSSQSIVHSNGHTYLEWYLQKPGQSPKLLIYKVSNRFSGVPDRFSGSGSGTDFTLKISRVEAEDLGVYYCFQGSHVPYTFGGGTKLEIK

SEQ ID NO: 510 - protein, 112 aa, synthetic construct
  REGION 1..112: note = mice-169
DVVMTQTPLSLPVSLGDQASISCRPSQTIVHSNGDTYLDWFLQKPGQSPKLLIYKVSNRFSGVPDRFSGSGSGTDFTLKISRVEAEDLGVYYCFQGSHVPYTFGGGTRLEIK

SEQ ID NO: 511 - protein, 112 aa, synthetic construct
  REGION 1..112: note = mice-170
DVVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGDGTKLEIK

SEQ ID NO: 512 - protein, 112 aa, synthetic construct
  REGION 1..112: note = mice-171
DVVMTQTPLSLPVSLGDQASISCRSSQSIVHSNGDTYLDWFLQKPGQSPKLLIYKVSNRFSGVPDRFSGSGSGTDFTLKITRVESEDLGVYYCFQGSHDPYTFGGGTKLEIK

SEQ ID NO: 513 - protein, 112 aa, synthetic construct
  REGION 1..112: note = mice-172
DVVMTQTPLSLPVSLGDQASISCRSSQSVVHNNGNTYLEWYLQKPGQSPKLLIYKVSNRFSGVPDRFSGSGSGTDFTLKISRVEAEDLGVYYCFQGSHVPYTFGGGTKLELK

SEQ ID NO: 514 - protein, 112 aa, synthetic construct
  REGION 1..112: note = mice-173
DVVMTQTPLSLPVSLGDQASISCRSSQSIVHSNGDTYLEWYLQKPGQSPKLLIYKVSNRFSGVPDRFSGSGSGTDFTLKISRVEAEDLGVYYCFQGSHVPYTFGGGTKLEIK

SEQ ID NO: 515 - protein, 112 aa, synthetic construct
  REGION 1..112: note = mice-174
DVVMTQTPLSLPVSLGDQASISCRSSQSIVHTNGDTYLDWYLQKPGQSPKLLIYKVSNRFSGVPDRFSGSGSGTDFTLKISRVEAEDLGVYYCFQGSHVPYTFGGGTKLEIK

SEQ ID NO: 516 - protein, 112 aa, synthetic construct
  REGION 1..112: note = mice-175
DVVMTQTPLSLPVSLGDQASISCRSSQSIVHDNGDTYLEWYLQKPGQSPKLLIYKVSNRFSGVPDRFSGSGSGTDFTLKINRVEAEDLGVYYCFQGSHVPYTFGGGTKLEIK

SEQ ID NO: 517 - protein, 112 aa, synthetic construct
  REGION 1..112: note = mice-176
DVVMTQTPLSLPVSLGDQASISCRSSQNIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 518 - protein, 111 aa, synthetic construct
  REGION 1..111: note = mice-177
DIVLTQSPASLAVSLGQRATISCRASESVDDYGISFMHWYQQKPGQPPKLLIYRASNLESGIPARFSGSGSRTDFTLTIDPVETDDVATYYCQQSYEDPPTFGAGTKLEIK

SEQ ID NO: 519 - protein, 111 aa, synthetic construct
  REGION 1..111: note = mice-178
DIVLTQSPASLAVSLGQRATISCRASESVDNYGISFMHWYQQKPGQPPKLLVYRASNLESGIPARFSGSGSRTDFTLTINPVETDDVATYYCQQSNKDPPTFGGGTKLEIK

SEQ ID NO: 520 - protein, 111 aa, synthetic construct
  REGION 1..111: note = mice-179
DIVLTQSPASLAVSLGQRATISCRASESVDNYGISFMHWYQQKPGQPPKLLIYRASNLESGIPARFSGSGSRTDFTLTINPVETDDVATYYCQQSNKDPPTFGAGTKLELK

SEQ ID NO: 521 - protein, 110 aa, synthetic construct
  REGION 1..110: note = mice-180
DIVLTQSPASLAVSLGQRATISCRASKSVSTSSYSYMHWYQQKPGQPPKLLIKYASNLESGVPARFSGSGSGTDFTLNIHPVEEEDAATYYCQHSREFPTFGGGTKLEIK

SEQ ID NO: 522 - protein, 113 aa, synthetic construct
  REGION 1..113: note = mice-181
DVVMTQTPLSLPVSLGDQASISCRSSQSIVHSNGDTYLDWYLEKPGQSPKLLIYKVSNRFSGVPDRFSGSGSGTDFTLKISRVEAEDLGVYYCFQGSHVPYTFGGGTKLEIKR

SEQ ID NO: 523 - protein, 112 aa, synthetic construct
  REGION 1..112: note = mice-182
DVVMTQTPLSLPVSLGDQASISCRSSQSIVHNNGDTYLDWYLQKPGQSPKLLIYKVSNRFSGVPDRFSGSGSGTDFTLKISRVEAEDLGVYYCFQGSHVPYTFGGGTKLELK

SEQ ID NO: 524 - protein, 112 aa, synthetic construct
  REGION 1..112: note = mice-183
DVVMTQTPLSLPVSLGDQASISCRSSQSIVHGNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 525 - protein, 112 aa, synthetic construct
  REGION 1..112: note = mice-184
DVVMTQTPLSLPVSLGDQASISCRSSQNIIHSNGDTYLDWFLQKPGQSPKLLIYKVSNRFSGVPDRFSGSGSGTDFTLKISRVEAEDLGVYYCFQGSHVPYTFGGGTKLEIK

SEQ ID NO: 526 - protein, 113 aa, synthetic construct
  REGION 1..113: note = mice-185
DVVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGRGDQAGNKT

SEQ ID NO: 527 - protein, 112 aa, synthetic construct
  REGION 1..112: note = mice-186
DVVMTQTPLSLPVSLGDQASISCRSSQSIVHSNGDTYLEWYLQKPGQSPKLLIYKVSNRFSGVPDRFSGSGSGTDFTLKISRVEAEDLGIYYCFQGSHVPYTFGGGTKLEIK

SEQ ID NO: 528 - protein, 112 aa, synthetic construct
  REGION 1..112: note = mice-187
DVVMTQTPLSLPVSLGNQASISCRSSQDIVHTNGDTYLDWYLQKPGQSPKLLIYKVSNRFSGVPDRFSGSGSGTDFTLKINRVEAEDLGVYYCFQGSHVPYTFGGGTKLELK

SEQ ID NO: 529 - protein, 112 aa, synthetic construct
  REGION 1..112: note = mice-188
DVVMTQTPLSLPVSLGDQASISCRSSQSIVHSNGDTYLDWFLQKPGQSPKLLIYKVSNRFSGVPDRFSGSGSGTDFTLKISRVEAEDLGVYYCFQGSHVPYTFGGGTKLELK

SEQ ID NO: 530 - protein, 112 aa, synthetic construct
  REGION 1..112: note = mice-189
DVVMTQTPLYLPVSLGDQASISCRSSQSIVHSNGDTYLDWFLQKPGQSPKLLIYKVSNRFSGVPDRFSGSGSGTDFTLKISRVEAEDLGVYYCFQGSHVPYTFGGGTKLELK

SEQ ID NO: 531 - protein, 112 aa, synthetic construct
  REGION 1..112: note = mice-190
DVVMTQTPLSLPVSLGDQASISCRSSQTIVHSNGDTYLDWYLQKPGQSPKLLISKVSNRFSGVPDRFSGSGSGTDFTLKISSVEAEDLGVYYCFQGSHVPYTFGGGTKLEIK

SEQ ID NO: 532 - protein, 112 aa, synthetic construct
  REGION 1..112: note = mice-191
DVVMTQTPLSLPVSLADQASISCRSSQSIVHDNGDTYLEWYLQKPGQSPKLLIYKVSNRFSGVPDRFSGSGSGTDFTLKISRVEAEDLGVYYCFQGSHVPYTFGGGTKLEIK

SEQ ID NO: 533 - protein, 112 aa, synthetic construct
  REGION 1..112: note = mice-192
DVVMTQTPLSLPVSLGDQASISCRSSQSIVHNNGDTYLDWYLEKPGQSPKLLIYKVSNRFSGVPDRFSGSGSGTDFTLKISRVEAEDLGVYYCFQGSHVPYTFGGGTKLEIK

SEQ ID NO: 534 - protein, 112 aa, synthetic construct
  REGION 1..112: note = mice-193
DVVMTQTPLSLPVSLGDQASISCRSSQNIVHNNGDTYLDWFLQKPGQSPKLLIYKVSNRFSGVPDRFSGSGSGTDFTLKISRVEAEDLGVYYCFQGSHVPYTFGGGTKLELK

SEQ ID NO: 535 - protein, 112 aa, synthetic construct
  REGION 1..112: note = mice-194
DVVMTQTPLSLPVSLGDQASISCKSSQSIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 536 - protein, 112 aa, synthetic construct
  REGION 1..112: note = mice-195
DVVMTQTPLSLPVSLGDQASISCRSSQNIVHSNGDTYLEWYLQKPGQSPKLLIYKVSNRFSGVPDRFSGSGSGTDFTLKISRVEAEDLGVYYCFQGSYVPYTFGGGTKLEIK

SEQ ID NO: 537 - protein, 112 aa, synthetic construct
  REGION 1..112: note = mice-196
DVVMTQTPLSLPVSLGDQASISCRSSQNIVHSNGDTYLEWYLQKPGQSPKLLIYKVSNRFSGVPDRFSGSGSGTDFTLKISRVEAEDLGVYYCFQGSHVPYTFGGGTKLEIK

SEQ ID NO: 538 - protein, 112 aa, synthetic construct
  REGION 1..112: note = mice-197
DVVMTQTPLSLPVSLGDQASISCRSSQNIVHSNGDTYLDWFLQKPGQSPKLLIYKVSNRFSGVPDRFSGSGSGTDFTLKISRVEAEDLGVYYCFQGSHVPYTFGGGTKLEIK

SEQ ID NO: 539 - protein, 112 aa, synthetic construct
  REGION 1..112: note = mice-198
DVVMTQTPLSLPVSLGDQASISCRSSQSIVHSNGDTYLDWFLQKPGQSPKLLIYKVSNRFSGVPDRFSGSGSGTDFTLKISRVESEDLGVYYCFQGSHDPYTFGGGTKLEIK

SEQ ID NO: 540 - protein, 112 aa, synthetic construct
  REGION 1..112: note = mice-199
DVVMTQTPLSLPVSLGDQASISCRSSQSIVHSNGNTYLEWYLQKPGQSPKLLIYKVSDRFSGVPDRFSGSGSGTDFTLKISRVEAEDLGVYYCFQGSHVPYTFGGGTKLEIK

SEQ ID NO: 541 - protein, 112 aa, synthetic construct
  REGION 1..112: note = mice-200
DVVMTQTPLSLPVSLGHQASISCRSSQSIVHSNGNTYLEWYLQKPGQSPKLLIYKVSNRFSGVPDRFSGSGSGTDFTLKISRVEAEDLGVYYCFQGSHVPYTFGGGTKLELK

SEQ ID NO: 542 - protein, 113 aa, synthetic construct
  REGION 1..113: note = mice-201
DVVMTQTPLSLPVSLADQASISCRSSQSIVHDNGDTYLEWYLQKPGQSPKLLIYKVSNRFSGVPDRFSGSGSGTDFTLKISRVEAEDLGVYYCFQGSHVPYTFGWRDQAGNKT

SEQ ID NO: 543 - protein, 112 aa, synthetic construct
  REGION 1..112: note = mice-202
DVVMTQTPLSLPVSLGDQASISCRSSQSIVHSNGNTYLEWYLQKPGQSPKLLIYKASNRFSGVPDRFSGSGSGTDFTLRISSVEAEDLGVYYCFQGSHVPYTFGGGTKLEIK

SEQ ID NO: 544 - protein, 112 aa, synthetic construct
  REGION 1..112: note = mice-203
DVVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 545 - protein, 112 aa, synthetic construct
  REGION 1..112: note = mice-204
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPFTFGSGTKLEIK

SEQ ID NO: 546 - protein, 106 aa, synthetic construct
  REGION 1..106: note = mice-205
EIVLTQSPALMSASPGEKVTITCSASSSVSYMHWFQQKPGTSPKLWIYSTSNLASGVPARFSGSGSGTSYSLTISRMEAEDAATYYCQQRSSYPFTFGSGTKLEIK

SEQ ID NO: 547 - protein, 113 aa, synthetic construct
  REGION 1..113: note = mice-206
DVVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKR

SEQ ID NO: 548 - protein, 112 aa, synthetic construct
  REGION 1..112: note = mice-207
DVVMTQTPLSLPVSLGDQASISCRSSQNIVHSNGDTYLDWYLQKPGQSPKLLISKVSNRFSGVPDRFSGSGSGTDFTLKISRVEAEDLGVYYCFQGSHVPYTFGGGTKLEIK

SEQ ID NO: 549 - protein, 112 aa, synthetic construct
  REGION 1..112: note = mice-208
DVVMTQTPLSLPVSLGDQASISCRSSQNIVHSNGDTYLDWFLQKPGQSPKLLISKVSNRFSGVPDRFSGSGSGTDFTLKISRVEAEDLGVYYCFQGSHVPYTFGGGTKLEIK

SEQ ID NO: 550 - protein, 112 aa, synthetic construct
  REGION 1..112: note = mice-209
DVVMTQTPLSLPVSLGDQASISCRSSQSIVHSNGDTYLDWFLQKPGQSPKLLIYKVSNRFSGVPDRFSGSGSGTDFTLKISRVEAEDLGVYYCFQGSHVPYTFGGGTKLEIK

SEQ ID NO: 551 - protein, 112 aa, synthetic construct
  REGION 1..112: note = mice-210
DVVMTQTPLSLPVSLGDQASISCRSSQNIVHSNGDTYLDWFLQKPGQSPKLLIYKVSNRFSGVPDRFSGSGSGTDFTLKISRVEAEDLGVYYCFQGSHVPYTFGGGTKLELK

SEQ ID NO: 552 - protein, 112 aa, synthetic construct
  REGION 1..112: note = mice-211
DVVMTQTPLSLPVSLADQASISCRSSQSIVHDNGDTYLEWYLQKPGQSPRLLIYKVSNRFSGVPDRFSGSGSGTDFTLKINRVEAEDLGVYYCFQGSHVPYTFGGGTKLEIK

SEQ ID NO: 553 - protein, 112 aa, synthetic construct
  REGION 1..112: note = mice-212
DVVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 554 - protein, 112 aa, synthetic construct
  REGION 1..112: note = mice-213
DVVMTQTPLSLPVSLGDQASISCRSSQSIVHNNGDTYLEWYLQKPGQSPKLLIYKVSNRFSGVPDRFSGSGSGTDFTLKISRVEAEDLGVYYCFQGSHVPYTFGGGTKLEIK

SEQ ID NO: 555 - protein, 112 aa, synthetic construct
  REGION 1..112: note = mice-214
DVVMTQTPLSLPVSLGDQASISCRSSQNIVHTNGDTYLDWYLQKPGQSPKLLIYKVSNRFSGVPDRFSGSGSGTDFTLKISRVEAEDLGVYYCFQGSHVPYTFGGGTKLELK

SEQ ID NO: 556 - protein, 112 aa, synthetic construct
  REGION 1..112: note = mice-215
DVVMTQTPLSLPVSLGDQASISCRSSQTIVHSNGNTYLDWYLQKPGHSPQLLIYKVSNRFSGVPDRFSGSGSGTDFTLKISRVEAEDLGVYYCFQGSHVPYTFGGGTKLEIK

SEQ ID NO: 557 - protein, 112 aa, synthetic construct
  REGION 1..112: note = mice-216
DVVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGSGTKLEIK

SEQ ID NO: 558 - protein, 112 aa, synthetic construct
  REGION 1..112: note = mice-217
DVVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLELK

SEQ ID NO: 559 - protein, 112 aa, synthetic construct
  REGION 1..112: note = mice-218
DVVMTQTPLSLPVSLGDQASISCRSSQNIVHSNGDTYLEWYLQKPGQSPKLLIYKVSSRFSGVPDRFSGSGSGTDFTLKISRVEAEDLGIYYCFQGSHVPYTFGGGTKLEIK

SEQ ID NO: 560 - protein, 106 aa, synthetic construct
  REGION 1..106: note = mice-219
EILLTQSPAIIAASPGEKVTITCSASSSVSYMNWYQQKPGSSPKIWIYGISNLASGVPARFSGSGSGTSFSFTINSMEAEDVATYYCQQRSSYPFTFGSGTKLEIK

SEQ ID NO: 561 - protein, 112 aa, synthetic construct
  REGION 1..112: note = mice-220
DVLMTQAPLSLPVSLGDQASISCRSNQTIVHSNGNTYLEWYLQKPGQSPKLLIYKVSNRFSGVPDRFSGSGSGTDFTLKISGVEAEDLGVYYCFQGSHVPLTFGAGTKLELN

SEQ ID NO: 562 - protein, 106 aa, synthetic construct; identical to SEQ ID NO: 221
  REGION 1..106: note = mice-221

SEQ ID NO: 563 - protein, 107 aa, synthetic construct
  REGION 1..107: note = mice-222
DILMTQSPSSMSVSLGDTVSITCHASQDIFSYIGWLQQKPGKSFKGLIHHGTNLEDGVPSRFRGNGSGADYSLTISSLESEDFADYYCVQYAQFPYTFGGGTKLEIK

SEQ ID NO: 564 - protein, 113 aa, synthetic construct
  REGION 1..113: note = mice-223
DIVMTQSPSSLRVSAGEKVTMSCKSSQSLLNSGNQKNYLAWYQQKPGQPPKLLIYGASTRESGVPDRFTGSGSGTDFTLTISSVQAEDLAVYYCQNDHSYPYTFGGGTKLEIK

SEQ ID NO: 565 - protein, 108 aa, synthetic construct; identical to SEQ ID NO: 308
  REGION 1..108: note = mice-224

(19) United States
(12) Patent Application Publication
(10) Pub. No.: US 2026/0062492 A1
(43) Pub. Date: Mar. 5, 2026

(54) ANTIBODIES AGAINST TL1A AND USES THEREOF

(71) Applicant: Earendil Labs Inc., Wilmington, DE (US)

(72) Inventors: Jian HUANG, Suzhou, Jiangsu (CN); Zhenping ZHU, Suzhou, Jiangsu (CN); Jian PENG, Suzhou, Jiangsu (CN); Liang TIAN, Suzhou, Jiangsu (CN); Chenpeng SU, Suzhou, Jiangsu (CN); Hao RAN, Suzhou, Jiangsu (CN)

(21) Appl. No.: 19/312,502

(22) Filed: Aug. 28, 2025

(30) Foreign Application Priority Data
Aug. 28, 2024 (CN) 202411195305.2
Feb. 5, 2025 (CN) 202510130736.9

(51) Int. Cl. C07K 16/28; A61K 39/00; A61P 1/00

(52) U.S. Cl. CPC C07K 16/2875; A61P 1/00; A61K 2039/505; C07K 2317/33; C07K 2317/52; C07K 2317/565; C07K 2317/76; C07K 2317/92; C07K 2317/94

(57) ABSTRACT

The present disclosure provides an isolated antigen-binding protein specifically binding to TNF-like protein A (TL1A), comprising a heavy chain variable region (VH) comprising at least one heavy chain complementarity-determining region (HCDR) comprising an amino acid sequence shown in SEQ ID NO:1, SEQ ID NO:5, or SEQ ID NO:14.

### CROSS-REFERENCE TO RELATED APPLICATIONS

This application claims priority to Chinese patent application No. 2024111953052, filed Aug. 28, 2024, the disclosure of which is herein incorporated by reference in its entirety. This application also claims priority to Chinese patent application No. 2025101307369, filed Feb. 5, 2025, the disclosure of which is herein incorporated by reference in its entirety.

### FIELD OF THE INVENTION

The present application relates to the field of biomedicine, and specifically to a TL1A antigen-binding protein.

### INCORPORATION BY REFERENCE OF SEQUENCE LISTING

The content of the electronic sequence listing (HEXO_001_02US_SubSeqList_ST26.xml: Size: 795,360 bytes; and Date of Creation: Sep. 29, 2025) is herein incorporated by reference in its entirety.

### BACKGROUND OF THE INVENTION

TL1A, also known as TNFSF15, is a member of the tumor necrosis factor (TNF) ligand superfamily, also known as vascular growth inhibitory factor (VEGI), which is a cytokine secreted mainly by vascular endothelial cells. TL1A possesses physiological activities such as activation of T-cells and promotion of secretion of inflammatory factors, and plays an important role in immune regulation and inflammatory diseases. Inflammation-related diseases can be treated by targeting antibodies to this target, and constructing effective antibodies for new drug development remains the biggest challenge.

### SUMMARY OF THE INVENTION

In one aspect, the present application provides an isolated antigen-binding protein capable of specifically binding to TNF-like protein A (TL1A) comprising a heavy chain variable region (VH) comprising at least one heavy chain complementarity-determining region (the HCDR) comprising an amino acid sequence shown in SEQ ID NO: 1, SEQ ID NO:5, or SEQ ID NO: 14.

In some embodiments, the VH comprises an HCDR3 including an amino acid sequence as set forth in SEQ ID NO:14.

In some embodiments, the HCDR3 comprises an amino acid sequence as set forth in any one of SEQ ID NOs: 15-18.

I In some embodiments, the VH comprises an HCDR2 including an amino acid sequence as set forth in SEQ ID NO:5.

In some embodiments, the HCDR2 comprises an amino acid sequence as set forth in any one of SEQ ID NOs: 6-13.

In some embodiments, the VH comprises an HCDR1 including an amino acid sequence as set forth in SEQ ID NO:1.

In some embodiments, the HCDR1 comprises an amino acid sequence as set forth in any one of SEQ ID NO:2-4.

In some embodiments, the VH comprises the HCDR1 as set forth in SEQ ID NO:2, the HCDR2 as set forth in SEQ ID NO:6, and the HCDR3 as set forth in SEQ ID NO:15.

In some embodiments, the VH comprises the HCDR1 as set forth in SEQ ID NO:2, the HCDR2 as set forth in SEQ ID NO:7, and the HCDR3 as set forth in SEQ ID NO:15.

In some embodiments, the VH comprises the HCDR1 as set forth in SEQ ID NO:2, the HCDR2 as set forth in SEQ ID NO:8, and the HCDR3 as set forth in SEQ ID NO:15.

In some embodiments, the VH comprises the HCDR1 as set forth in SEQ ID NO:3, the HCDR2 as set forth in SEQ ID NO:6, and the HCDR3 as set forth in SEQ ID NO:15.

In some embodiments, the VH comprises the HCDR1 as set forth in SEQ ID NO:2, the HCDR2 as set forth in SEQ ID NO:6, and the HCDR3 as set forth in SEQ ID NO:16.

In some embodiments, the VH comprises the HCDR1 as set forth in SEQ ID NO:2, the HCDR2 as set forth in SEQ ID NO:6, and the HCDR3 as set forth in SEQ ID NO:17.

In some embodiments, the VH comprises the HCDR1 as set forth in SEQ ID NO:2, the HCDR2 as set forth in SEQ ID NO:6, and the HCDR3 as set forth in SEQ ID NO:18.

In some embodiments, the VH comprises the HCDR1 as set forth in SEQ ID NO:4, the HCDR2 as set forth in SEQ ID NO:6, and the HCDR3 as set forth in SEQ ID NO:15.

In some embodiments, the VH comprises the HCDR1 as set forth in SEQ ID NO:2, the HCDR2 as set forth in SEQ ID NO:9, and the HCDR3 as set forth in SEQ ID NO:15.

In some embodiments, the VH comprises the HCDR1 as set forth in SEQ ID NO:2, the HCDR2 as set forth in SEQ ID NO: 10, and the HCDR3 as set forth in SEQ ID NO: 15.

In some embodiments, the VH comprises the HCDR1 as set forth in SEQ ID NO:2, the HCDR2 as set forth in SEQ ID NO: 11, and the HCDR3 as set forth in SEQ ID NO: 15.

In some embodiments, the VH comprises the HCDR1 as set forth in SEQ ID NO:2, the HCDR2 as set forth in SEQ ID NO: 12, and the HCDR3 as set forth in SEQ ID NO: 15.

In some embodiments, the VH comprises the HCDR1 as set forth in SEQ ID NO:2, the HCDR2 as set forth in SEQ ID NO: 13, and the HCDR3 as set forth in SEQ ID NO: 15.

In some embodiments, the VH comprises the HCDR1 as set forth in SEQ ID NO:3, the HCDR2 as set forth in SEQ ID NO:6, and the HCDR3 as set forth in SEQ ID NO:16.

In some embodiments, the VH comprises the HCDR1 as set forth in SEQ ID NO:3, the HCDR2 as set forth in SEQ ID NO:6, and the HCDR3 as set forth in SEQ ID NO:17.

In some embodiments, the VH comprises the HCDR1 as set forth in SEQ ID NO:3, the HCDR2 as set forth in SEQ ID NO:9, and the HCDR3 as set forth in SEQ ID NO: 15.

In some embodiments, the VH comprises the HCDR1 as set forth in SEQ ID NO:3, the HCDR2 as set forth in SEQ ID NO: 10, and the HCDR3 as set forth in SEQ ID NO: 15.

In some embodiments, the VH comprises the HCDR1 as set forth in SEQ ID NO:3, the HCDR2 as set forth in SEQ ID NO: 11, and the HCDR3 as set forth in SEQ ID NO: 15.

In some embodiments, the VH comprises the HCDR1 as set forth in SEQ ID NO:2, the HCDR2 as set forth in SEQ ID NO:9, and the HCDR3 as set forth in SEQ ID NO: 16.

In some embodiments, the VH comprises the HCDR1 as set forth in SEQ ID NO:2, the HCDR2 as set forth in SEQ ID NO: 10, and the HCDR3 as set forth in SEQ ID NO: 16.

In some embodiments, the VH comprises the HCDR1 as set forth in SEQ ID NO:2, the HCDR2 as set forth in SEQ ID NO: 11, and the HCDR3 as set forth in SEQ ID NO: 16.

In some embodiments, the VH comprises the HCDR1 as set forth in SEQ ID NO:2, the HCDR2 as set forth in SEQ ID NO: 12, and the HCDR3 as set forth in SEQ ID NO: 16.

In some embodiments, the VH comprises the HCDR1 as set forth in SEQ ID NO:2, the HCDR2 as set forth in SEQ ID NO: 10, and the HCDR3 as set forth in SEQ ID NO: 17.

In some embodiments, the VH comprises an HFR1 including an amino acid sequence as set forth in SEQ ID NO:19.

In some embodiments, the HFR1 is selected from an amino acid sequence shown in any one of the SEQ ID NOs: 20-28.

In some embodiments, the VH comprises the HFR2 including an amino acid sequence as set forth in SEQ ID NO:29.

In some embodiments, the HFR2 is selected from an amino acid sequence shown in any one of the SEQ ID NOs: 30-32.

In some embodiments, the VH comprises HFR3 including an amino acid sequence as set forth in SEQ ID NO:33.

In some embodiments, the HFR3 is selected from an amino acid sequence shown in any one of SEQ ID NOs: 34-40.

In some embodiments, the VH contains HFR4 including an amino acid sequence shown in SEQ ID NO: 41.

In some embodiments, the VH comprises HFR1 as set forth in SEQ ID NO:20, HFR2 as set forth in SEQ ID NO:30, HFR3 as set forth in SEQ ID NO:34, and HFR4 as set forth in SEQ ID NO:41.

In some embodiments, the VH comprises HFR1 as set forth in SEQ ID NO:21, HFR2 as set forth in SEQ ID NO:31, HFR3 as set forth in SEQ ID NO:35, and HFR4 as set forth in SEQ ID NO:41.

In some embodiments, the VH comprises HFR1 as set forth in SEQ ID NO:22, HFR2 as set forth in SEQ ID NO:32, HFR3 as set forth in SEQ ID NO:35, and HFR4 as set forth in SEQ ID NO:41.

In some embodiments, the VH comprises HFR1 as set forth in SEQ ID NO:23, HFR2 as set forth in SEQ ID NO:31, HFR3 as set forth in SEQ ID NO:3636NO: 35, and HFR4 as set forth in SEQ ID NO: 41.

In some embodiments, the VH comprises HFR1 as set forth in SEQ ID NO:23, HFR2 as set forth in SEQ ID NO:31, HFR3 as set forth in SEQ ID NO:37, and HFR4 as set forth in SEQ ID NO:41.

In some embodiments, the VH comprises HFR1 as set forth in SEQ ID NO:23, HFR2 as set forth in SEQ ID NO:31, HFR3 as set forth in SEQ ID NO:35, and HFR4 as set forth in SEQ ID NO:41.

In some embodiments, the VH comprises HFR1 as set forth in SEQ ID NO:24, HFR2 as set forth in SEQ ID NO:31, HFR3 as set forth in SEQ ID NO:38, and HFR4 as set forth in SEQ ID NO:41.

In some embodiments, the VH comprises HFR1 as set forth in SEQ ID NO: 25, HFR2 as set forth in SEQ ID NO: 31, HFR3 as set forth in SEQ ID NO: 37, and HFR4 as set forth in SEQ ID NO: 41.

In some embodiments, the VH comprises HFR1 as set forth in SEQ ID NO:25, HFR2 as set forth in SEQ ID NO:31, HFR3 as set forth in SEQ ID NO:39, and HFR4 as set forth in SEQ ID NO:41.

In some embodiments, the VH comprises HFR1 as set forth in SEQ ID NO:25, HFR2 as set forth in SEQ ID NO:31, HFR3 as set forth in SEQ ID NO:40, and HFR4 as set forth in SEQ ID NO:41.

In some embodiments, the VH comprises HFR1 as set forth in SEQ ID NO:26, HFR2 as set forth in SEQ ID NO:31, HFR3 as set forth in SEQ ID NO:35, and HFR4 as set forth in SEQ ID NO:41.

In some embodiments, the VH comprises HFR1 as set forth in SEQ ID NO:25, HFR2 as set forth in SEQ ID NO:31, HFR3 as set forth in SEQ ID NO:35, and HFR4 as set forth in SEQ ID NO:41.

In some embodiments, the VH comprises HFR1 as set forth in SEQ ID NO: 27, HFR2 as set forth in SEQ ID NO: 31, HFR3 as set forth in SEQ ID NO: 35, and HFR4 as set forth in SEQ ID NO: 41.

In some embodiments, the VH comprises HFR1 as set forth in SEQ ID NO:28, HFR2 as set forth in SEQ ID NO:31, HFR3 as set forth in SEQ ID NO:35, and HFR4 as set forth in SEQ ID NO:41.

In some embodiments, the isolated antigen-binding proteins further comprises a light chain variable region (VL) that comprises at least one light chain complementarity-determining region (LCDR) comprising an amino acid sequence selected from SEQ ID NO:42, SEQ ID NO:43, and SEQ ID NO: 44.

In some embodiments, the VL comprises LCDR3 including an amino acid sequence as set forth in SEQ ID NO:44.

In some embodiments, the VL comprises LCDR2 including an amino acid sequence as set forth in SEQ ID NO:43.

In some embodiments, the VL comprises LCDR1 including an amino acid sequence as set forth in SEQ ID NO:42.

in some embodiments, the VL comprises the LFR1 including an amino acid sequence as set forth in SEQ ID NO:45.

In some embodiments, the LFR1 comprises an amino acid sequence as set forth in any one of SEQ ID NOs: 46-47.

In some embodiments, the VL comprises LFR2, which comprises the amino acid sequence of SEQ ID NO:48.

In some embodiments, the LFR2 comprises an amino acid sequence shown in any one of the SEQ ID NOs: 49-52.

In some embodiments, the VL contains the LFR3, which comprises the amino acid sequence of SEQ ID NO:53.

In some embodiments, the LFR3 comprises an amino acid sequence as set forth in any one of SEQ ID NOs: 54-57.

In some embodiments, the VL contains the amino acid sequence of the LFR4, which comprises SEQ ID NO:58.

In some embodiments, the LFR1 comprises an amino acid sequence shown in any one of the SEQ ID NOs: 59-61.

In some embodiments, the VL comprises the LFR1 as set forth in SEQ ID NO:46, the LFR2 as set forth in SEQ ID NO:49, the LFR3 as set forth in SEQ ID NO:54, and the LFR4 as set forth in SEQ ID NO:59.

In some embodiments, the VL comprises the LFR1 as set forth in SEQ ID NO: 47, the LFR2 as set forth in SEQ ID NO: 50, the LFR3 as set forth in SEQ ID NO: 55, and the LFR4 as set forth in SEQ ID NO: 60.

In some embodiments, the VL comprises the LFR1 as set forth in SEQ ID NO:47, the LFR2 as set forth in SEQ ID NO:51, the LFR3 as set forth in SEQ ID NO:56, and the LFR4 as set forth in SEQ ID NO:61.

In some embodiments, the VL comprises the LFR1 as set forth in SEQ ID NO:47, the LFR2 as set forth in SEQ ID NO:52, the LFR3 as set forth in SEQ ID NO:57, and the LFR4 as set forth in SEQ ID NO:61.

In some embodiments, the VL comprises the LFR1 as set forth in SEQ ID NO: 47, the LFR2 as set forth in SEQ ID NO: 52, the LFR3 as set forth in SEQ ID NO: 56, and the LFR4 as set forth in SEQ ID NO: 61.

In some embodiments, the VH comprises sequences as set forth in any one of SEQ ID NOs: 62-99.

In some embodiments, the VL comprises sequences as set forth in any one of SEQ ID NOs: 102-106

In some embodiments, the isolated antigen-binding protein includes VH including HCDR1 as set forth in SEQ ID NO: 2, HCDR2 as set forth in SEQ ID NO: 6, HCDR3 as set forth in SEQ ID NO: 15, and VL including LCDR1 as set forth in SEQ ID NO: 42, LCDR2 as set forth in SEQ ID NO: 43, and LCDR3 as set forth in SEQ ID NO: 44.

In some embodiments, the isolated antigen-binding protein includes VH as set forth in SEQ ID NO: 70 and VL as set forth in SEQ ID NO: 106.

In another aspect, the present application provides an isolated antigen-binding protein capable of specifically binding to TNF-like protein A (TL1A) comprising a heavy chain variable region (VH) comprising at least one heavy chain complementarity-determining region (HCDR) comprising an amino acid sequence selected from SEQ ID NO: 107, SEQ ID NO:115, and SEQ ID NO:120.

In some embodiments, the VH comprises HCDR3 including an amino acid sequence as set forth in SEQ ID NO:120.

In some embodiments, the VH comprises HCDR2 including an amino acid sequence as set forth in SEQ ID NO:115.

In some embodiments, the HCDR2 comprises an amino acid sequence as set forth in any one of SEQ ID NOs: 116-119.

In some embodiments, the VH comprises HCDR1 including an amino acid sequence as set forth in SEQ ID NO:107.

In some embodiments, the HCDR1 comprises an amino acid sequence as set forth in any one of SEQ ID NOs: 108-114.

In some embodiments, the VH comprises the HCDR1 as set forth in SEQ ID NO:108, the HCDR2 as set forth in SEQ ID NO:116, and the HCDR3 as set forth in SEQ ID NO: 120.

In some embodiments, the VH comprises the HCDR1 as set forth in SEQ ID NO:109, the HCDR2 as set forth in SEQ ID NO:118, and the HCDR3 as set forth in SEQ ID NO: 120.

In some embodiments, the VH comprises the HCDR1 as set forth in SEQ ID NO: 108, the HCDR2 as set forth in SEQ ID NO: 119, and the HCDR3 AS SHOWN IN SEQ ID NO: 120.

In some embodiments, the VH comprises the HCDR1 as set forth in SEQ ID NO:110, the HCDR2 as set forth in SEQ ID NO:118, and the HCDR3 as set forth in SEQ ID NO: 120.

In some embodiments, the VH comprises the HCDR1 as set forth in SEQ ID NO:111, the HCDR2 as set forth in SEQ ID NO:117, and the HCDR3 as set forth in SEQ ID NO: 120.

In some embodiments, the VH comprises the HCDR1 as set forth in SEQ ID NO:112, the HCDR2 as set forth in SEQ ID NO:117, and the HCDR3 as set forth in SEQ ID NO: 120.

In some embodiments, the VH comprises the HCDR1 as set forth in SEQ ID NO:113, the HCDR2 as set forth in SEQ ID NO:117, and the HCDR3 as set forth in SEQ ID NO:120.

In some embodiments, the VH comprises the HCDR1 as set forth in SEQ ID NO:108, the HCDR2 as set forth in SEQ ID NO:117, and the HCDR3 as set forth in SEQ ID NO:120.

In some embodiments, the VH comprises the HCDR1 as set forth in SEQ ID NO:114, the HCDR2 as set forth in SEQ ID NO:117, and the HCDR3 as set forth in SEQ ID NO:120.

In some embodiments, the VH comprises HFR1 including an amino acid sequence as set forth in SEQ ID NO:121.

In some embodiments, the HFR1 is selected from an amino acid sequence shown in any one of SEQ ID NOs: 122-132.

In some embodiments, the VH comprises HFR2 including an amino acid sequence as set forth in SEQ ID NO:133.

In some embodiments, the HFR2 is selected from an amino acid sequence shown in any one of SEQ ID NOs: 134-135, 30.

In some embodiments, the VH comprises HFR3 including an amino acid sequence as set forth in SEQ ID NO:136.

In some embodiments, the HFR3 is selected from an amino acid sequence shown in any one of SEQ ID NOs: 137-148.

In some embodiments, the VH comprises HFR4 including an amino acid sequence shown in SEQ ID NO: 149.

In some embodiments, the HFR4 is selected from an amino acid sequence shown in any one of SEQ ID NOs: 150-153, 41.

In some embodiments, the VH comprises HFR1 as set forth in SEQ ID NO: 122, HFR2 as set forth in SEQ ID NO: 134, HFR3 as set forth in SEQ ID NO: 137, and HFR4 as set forth in SEQ ID NO: 150.

In some embodiments, the VH comprises HFR1 as set forth in SEQ ID NO: 123, HFR2 as set forth in SEQ ID NO: 135, HFR3 as set forth in SEQ ID NO: 138, and HFR4 as set forth in SEQ ID NO: 41.

In some embodiments, the VH comprises HFR1 as set forth in SEQ ID NO: 124, HFR2 as set forth in SEQ ID NO: 135, HFR3 as set forth in SEQ ID NO: 139, and HFR4 as set forth in SEQ ID NO: 151.

In some embodiments, the VH comprises HFR1 as set forth in SEQ ID NO: 125, HFR2 as set forth in SEQ ID NO: 135, HFR3 as set forth in SEQ ID NO: 142, and HFR4 as set forth in SEQ ID NO: 41.

In some embodiments, the VH comprises HFR1 as set forth in SEQ ID NO: 123, HFR2 as set forth in SEQ ID NO: 135, HFR3 as set forth in SEQ ID NO: 140, and HFR4 as set forth in SEQ ID NO: 41.

In some embodiments, the VH comprises HFR1 as set forth in SEQ ID NO: 123, HFR2 as set forth in SEQ ID NO: 135, HFR3 as set forth in SEQ ID NO: 141, and HFR4 as set forth in SEQ ID NO: 41.

In some embodiments, the VH comprises HFR1 as set forth in SEQ ID NO: 126, HFR2 as set forth in SEQ ID NO: 135, HFR3 as set forth in SEQ ID NO: 142, and HFR4 as set forth in SEQ ID NO: 152.

In some embodiments, the VH comprises HFR1 as set forth in SEQ ID NO: 127, HFR2 as set forth in SEQ ID NO: 30, HFR3 as set forth in SEQ ID NO: 143, and HFR4 as set forth in SEQ ID NO: 41.

In some embodiments, the VH comprises HFR1 as set forth in SEQ ID NO: 128, HFR2 as set forth in SEQ ID NO: 30, HFR3 as set forth in SEQ ID NO: 144, and HFR4 as set forth in SEQ ID NO: 41.

In some embodiments, the VH comprises HFR1 as set forth in SEQ ID NO: 129, HFR2 as set forth in SEQ ID NO: 30, HFR3 as set forth in SEQ ID NO: 145, and HFR4 as set forth in SEQ ID NO: 41.

In some embodiments, the VH comprises HFR1 as set forth in SEQ ID NO: 127, HFR2 as set forth in SEQ ID NO: 30, HFR3 as set forth in SEQ ID NO: 146, and HFR4 as set forth in SEQ ID NO: 41.

In some embodiments, the VH comprises HFR1 as set forth in SEQ ID NO: 125, HFR2 as set forth in SEQ ID NO: 135, HFR3 as set forth in SEQ ID NO: 142, and HFR4 as set forth in SEQ ID NO: 152.

In some embodiments, the VH comprises HFR1 as set forth in SEQ ID NO: 130, HFR2 as set forth in SEQ ID NO: 30, HFR3 as set forth in SEQ ID NO: 147, and HFR4 as set forth in SEQ ID NO: 41.

In some embodiments, the VH comprises HFR1 as set forth in SEQ ID NO: 131, HFR2 as set forth in SEQ ID NO: 135, HFR3 as set forth in SEQ ID NO: 148, and HFR4 as set forth in SEQ ID NO: 153.

In some embodiments, the VH comprises HFR1 as set forth in SEQ ID NO:132, HFR2 as set forth in SEQ ID NO:30, HFR3 as set forth in SEQ ID NO:145, and HFR4 as set forth in SEQ ID NO: 41.

In some embodiments, the isolated antigen-binding protein further comprises a light chain variable region (VL) that comprises at least one light chain complementarity-determining region (LCDR) comprising an amino acid sequence selected from SEQ ID NO:154, SEQ ID NO:159, and SEQ ID NO:163.

In some embodiments, the VL comprises LCDR3 including an amino acid sequence as set forth in SEQ ID NOs: 163.

In some embodiments, the LCDR3 comprises an amino acid sequence shown in any one of the SEQ ID NOs: 164-168.

1 In some embodiments, the VL comprises LCDR2 including an amino acid sequence as set forth in SEQ ID NO:159.

In some embodiments, the LCDR2 comprises an amino acid sequence shown in any one of the SEQ ID NOs: 160-162.

In some embodiments, the VL comprises LCDR1 including an amino acid sequence as set forth in SEQ ID NO:154.

In some embodiments, the LCDR1 comprises an amino acid sequence shown in any one of the SEQ ID NOs: 155-158.

In some embodiments, the VL comprises LCDR1 as set forth in SEQ ID NO: 157, LCDR2 as set forth in SEQ ID NO: 161 and LCDR3 as set forth in SEQ ID NO: 164.

In some embodiments, the VL comprises LCDR1 as set forth in SEQ ID NO: 157, LCDR2 as set forth in SEQ ID NO: 162 and LCDR3 as set forth in SEQ ID NO: 164.

In some embodiments, the VL comprises LCDR1 as set forth in SEQ ID NO: 156, LCDR2 as set forth in SEQ ID NO: 161 and LCDR3 as set forth in SEQ ID NO: 164.

In some embodiments, the VL comprises LCDR1 as set forth in SEQ ID NO: 158, LCDR2 as set forth in SEQ ID NO: 160 and LCDR3 as set forth in SEQ ID NO: 164.

In some embodiments, the VL comprises LCDR1 as set forth in SEQ ID NO: 155, LCDR2 as set forth in SEQ ID NO: 160 and LCDR3 as set forth in SEQ ID NO: 164.

In some embodiments, the VL comprises LCDR1 as set forth in SEQ ID NO: 156, LCDR2 as set forth in SEQ ID NO: 162, and LCDR3 as set forth in SEQ ID NO: 164.

In some embodiments, the VL comprises LCDR1 as set forth in SEQ ID NO: 155, LCDR2 as set forth in SEQ ID NO: 160 and LCDR3 as set forth in SEQ ID NO: 165.

In some embodiments, the VL comprises LCDR1 as set forth in SEQ ID NO: 155, LCDR2 as set forth in SEQ ID NO: 160 and LCDR3 as set forth in SEQ ID NO: 166.

In some embodiments, the VL comprises LCDR1 as set forth in SEQ ID NO: 155, LCDR2 as set forth in SEQ ID NO: 160 and LCDR3 as set forth in SEQ ID NO: 167.

In some embodiments, the VL comprises LCDR1 as set forth in SEQ ID NO: 155, LCDR2 as set forth in SEQ ID NO: 160 and LCDR3 as set forth in SEQ ID NO: 168.

In some embodiments, the VL comprises LFR1 including an amino acid sequence of SEQ ID NO: 169.

In some embodiments, the LFR1 comprises an amino acid sequence as set forth in SEQ ID NOs: 170-179.

In some embodiments, the VL comprises LFR2 including an amino acid sequence of SEQ ID NO: 180.

In some embodiments, the LFR2 comprises an amino acid sequence as set forth in any one of SEQ ID NOs: 181-188.

In some embodiments, the VL comprises LFR3 including an amino acid sequence of SEQ ID NO: 189.

In some embodiments, the LFR3 comprises an amino acid sequence shown in SEQ ID NOs: 190-197, 57.

In some embodiments, the VL comprises LFR4 including an amino acid sequence of SEQ ID NO: 198.

In some embodiments, the LFR4 comprises an amino acid sequence indicated in SEQ ID NOs: 199-201, 61.

In some embodiments, the VL comprises the LFR1 as set forth in SEQ ID NO: 170, the LFR2 as set forth in SEQ ID NO:181, the LFR3 as set forth in SEQ ID NO:190, and the LFR4 as set forth in SEQ ID NO:199.

In some embodiments, the VL comprises the LFR1 as set forth in SEQ ID NO: 171, the LFR2 as set forth in SEQ ID NO:182, the LFR3 as set forth in SEQ ID NO:191, and the LFR4 as set forth in SEQ ID NO:200.

In some embodiments, the VL comprises the LFR1 as set forth in SEQ ID NO: 172, the LFR2 as set forth in SEQ ID NO:183, the LFR3 as set forth in SEQ ID NO:192, and the LFR4 as set forth in SEQ ID NO:61.

In some embodiments, the VL comprises the LFR1 as set forth in SEQ ID NO: 174, the LFR2 as set forth in SEQ ID NO:184, the LFR3 as set forth in SEQ ID NO:57, and the LFR4 as set forth in SEQ ID NO:199.

In some embodiments, the VL comprises the LFR1 as set forth in SEQ ID NO: 175, the LFR2 as set forth in SEQ ID NO: 185, the LFR3 as set forth in SEQ ID NO: 193, and the LFR4 as set forth in SEQ ID NO: 199.

In some embodiments, the VL comprises the LFR1 as set forth in SEQ ID NO: 176, the LFR2 as set forth in SEQ ID NO: 186, the LFR3 as set forth in SEQ ID NO: 194, and the LFR4 as set forth in SEQ ID NO: 199.

In some embodiments, the VL comprises the LFR1 as set forth in SEQ ID NO: 177, the LFR2 as set forth in SEQ ID NO: 187, the LFR3 as set forth in SEQ ID NO: 195, and the LFR4 as set forth in SEQ ID NO: 199.

In some embodiments, the VL comprises the LFR1 as set forth in SEQ ID NO: 178, the LFR2 as set forth in SEQ ID NO: 188, the LFR3 as set forth in SEQ ID NO: 196, and the LFR4 as set forth in SEQ ID NO: 199.

In some embodiments, the VL comprises the LFR1 as set forth in SEQ ID NO: 179, the LFR2 as set forth in SEQ ID NO: 187, the LFR3 as set forth in SEQ ID NO: 195, and the LFR4 as set forth in SEQ ID NO: 199.

In some embodiments, the VL comprises the LFR1 as set forth in SEQ ID NO: 173, the LFR2 as set forth in SEQ ID NO:183, the LFR3 as set forth in SEQ ID NO:192, and the LFR4 as set forth in SEQ ID NO:201.

In some embodiments, the VH comprises sequence as set forth in any one of SEQ ID NOs: 202-220, 332-334.

In some embodiments, the VL comprises sequence as set forth in any one of SEQ ID NOs: 221-234, 335-338.

In another aspect, the present application provides an isolated antigen-binding protein capable of specifically binding to TNF-like protein A (TL1A) comprising a heavy chain variable region (VH) comprising at least one heavy chain complementarity-determining region (HCDR) comprising an amino acid sequence selected from an amino acid sequence shown in SEQ ID NO:242, SEQ ID NO: 238, and SEQ ID NO:235.

In some embodiments, the VH comprises the HCDR3 including an amino acid sequence as set forth in SEQ ID NO:242.

In some embodiments, the VH comprises the HCDR2 including an amino acid sequence as set forth in SEQ ID NO:238.

In some embodiments, the HCDR2 comprises an amino acid sequence as set forth in any one of SEQ ID NOs: 239-241.

in some embodiments, the VH comprises the HCDR1 including an amino acid sequence of SEQ ID NO: 235.

In some embodiments, the HCDR1 comprises an amino acid sequence as set forth in any one of SEQ ID NOs: 236-237.

In some embodiments, the VH comprises the HCDR1 as set forth in SEQ ID NO:108, the HCDR2 as set forth in SEQ ID NO:116, and the HCDR3 as set forth in SEQ ID NO:120.

In some embodiments, the VH comprises the HCDR1 as set forth in SEQ ID NO: 236, the HCDR2 as set forth in SEQ ID NO: 239, and the HCDR3 as set forth in SEQ ID NO: 242.

In some embodiments, the VH comprises the HCDR1 as set forth in SEQ ID NO: 236, the HCDR2 as set forth in SEQ ID NO: 240, and the HCDR3 as set forth in SEQ ID NO: 242.

In some embodiments, the VH comprises the HCDR1 as set forth in SEQ ID NO:237, the HCDR2 as set forth in SEQ ID NO:241, and HCDR3 as set forth in SEQ ID NO:242.

In some embodiments, the VH comprises HFR1 including an amino acid sequence as set forth in SEQ ID NO:243.

In some embodiments, the HFR1 is selected from an amino acid sequence shown in any one of SEQ ID NOs: 244-250.

In some embodiments, the VH comprises HFR2 including an amino acid sequence as set forth in SEQ ID NO:251.

In some embodiments, the HFR2 is selected from an amino acid sequence shown in any one of SEQ ID NOs: 252-255.

In some embodiments, the VH contains HFR3 including an amino acid sequence of SEQ ID NO: 256.

In some embodiments, the HFR3 is selected from an amino acid sequence shown in any one of SEQ ID NOs: 257-262.

In some embodiments, the VH comprises HFR4 including an amino acid sequence shown in SEQ ID NO:263.

In some embodiments, the HFR4 is selected from an amino acid sequence shown in any one of SEQ ID NOs: 264-268.

In some embodiments, the VH comprises HFR1 as set forth in SEQ ID NO: 244, HFR2 as set forth in SEQ ID NO: 252, HFR3 as set forth in SEQ ID NO: 257, and HFR4 as set forth in SEQ ID NO: 264.

In some embodiments, the VH comprises HFR1 as set forth in SEQ ID NO: 245, HFR2 as set forth in SEQ ID NO: 252, HFR3 as set forth in SEQ ID NO: 258, and HFR4 as set forth in SEQ ID NO: 265.

In some embodiments, the VH comprises HFR1 as set forth in SEQ ID NO: 246, HFR2 as set forth in SEQ ID NO: 253, HFR3 as set forth in SEQ ID NO: 259, and HFR4 as set forth in SEQ ID NO: 268.

In some embodiments, the VH comprises HFR1 as set forth in SEQ ID NO: 246, HFR2 as set forth in SEQ ID NO: 254, HFR3 as set forth in SEQ ID NO: 260, and HFR4 as set forth in SEQ ID NO: 266.

In some embodiments, the VH comprises HFR1 as set forth in SEQ ID NO: 247, HFR2 as set forth in SEQ ID NO: 254, HFR3 as set forth in SEQ ID NO: 261, and HFR4 as set forth in SEQ ID NO: 268.

In some embodiments, the VH comprises HFR1 as set forth in SEQ ID NO: 248, HFR2 as set forth in SEQ ID NO: 252, HFR3 as set forth in SEQ ID NO: 258, and HFR4 as set forth in SEQ ID NO: 265.

In some embodiments, the VH comprises HFR1 as set forth in SEQ ID NO: 246, HFR2 as set forth in SEQ ID NO: 255, HFR3 as set forth in SEQ ID NO: 260, and HFR4 as set forth in SEQ ID NO: 268.

In some embodiments, the VH comprises HFR1 as set forth in SEQ ID NO: 245, HFR2 as set forth in SEQ ID NO: 252, HFR3 as set forth in SEQ ID NO: 258, and HFR4 as set forth in SEQ ID NO: 267.

In some embodiments, the VH comprises HFR1 as set forth in SEQ ID NO: 249, HFR2 as set forth in SEQ ID NO: 254, HFR3 as set forth in SEQ ID NO: 260, and HFR4 as set forth in SEQ ID NO: 268.

In some embodiments, the VH comprises HFR1 as set forth in SEQ ID NO: 250, HFR2 as set forth in SEQ ID NO: 253, HFR3 as set forth in SEQ ID NO: 262, and HFR4 as set forth in SEQ ID NO: 266.

In some embodiments, the isolated antigen-binding protein further comprises a light chain variable region (VL) comprising at least one light chain complementarity-determining region (LCDR) comprising an amino acid sequence selected from SEQ ID NO: 269, SEQ ID NO: 274, and SEQ ID NO: 277.

In some embodiments, the VL comprises LCDR3 including an amino acid sequence as set forth in SEQ ID NO:277.

In some embodiments, the VL comprises LCDR2 including an amino acid sequence as set forth in SEQ ID NO:274.

In some embodiments, the LCDR2 comprises an amino acid sequence shown in any one of the SEQ ID NO:275-276.

In some embodiments, the VL comprises LCDR1 including an amino acid sequence as set forth in SEQ ID NO:269.

In some embodiments, the LCDR1 comprises an amino acid sequence shown in any one of the SEQ ID NOs: 270-273.

In some embodiments, the VL comprises LCDR1 as set forth in SEQ ID NO: 270, LCDR2 as set forth in SEQ ID NO: 275 and LCDR3 as set forth in SEQ ID NO: 277.

In some embodiments, the VL comprises LCDR1 as set forth in SEQ ID NO: 271, LCDR2 as set forth in SEQ ID NO: 275 and LCDR3 as set forth in SEQ ID NO: 277.

In some embodiments, the VL comprises LCDR1 as set forth in SEQ ID NO: 272, LCDR2 as set forth in SEQ ID NO: 275 and LCDR3 as set forth in SEQ ID NO: 277.

In some embodiments, the VL comprises LCDR1 as set forth in SEQ ID NO: 273, LCDR2 as set forth in SEQ ID NO: 275 and LCDR3 as set forth in SEQ ID NO: 277.

In some embodiments, the VL comprises LCDR1 as set forth in SEQ ID NO: 271, LCDR2 as set forth in SEQ ID NO: 276 and LCDR3 as set forth in SEQ ID NO: 277.

In some embodiments, the VL contains the LFR1 including an amino acid sequence of SEQ ID NO: 278.

In some embodiments, the LFR1 comprises an amino acid sequence shown in SEQ ID NOs: 47, 173, 279-283.

In some embodiments, the VL comprises the LFR2 including an amino acid sequence as set forth in SEQ ID NO:284.

In some embodiments, the LFR2 comprises an amino acid sequence shown in SEQ ID NOs: 52, 285-287.

In some embodiments, the VL comprises the LFR3 including an amino acid sequence as set forth in SEQ ID NO:288.

In some embodiments, the LFR3 comprises an amino acid sequence shown in SEQ ID NOs: 289-293, 57.

In some embodiments, the VL comprises the LFR4 including an amino acid sequence as set forth in SEQ ID NO:294.

In some embodiments, the LFR4 comprises an amino acid sequence shown in SEQ ID NO:201, 295-296.

In some embodiments, the VL comprises the LFR1 as set forth in SEQ ID NO: 170, the LFR2 as set forth in SEQ ID NO:181, the LFR3 as set forth in SEQ ID NO:190, and the LFR4 as set forth in SEQ ID NO:199.

In some embodiments, the VL comprises the LFR1 as set forth in SEQ ID NO: 171, the LFR2 as set forth in SEQ ID NO:182, the LFR3 as set forth in SEQ ID NO:191, and the LFR4 as set forth in SEQ ID NO:200.

In some embodiments, the VL comprises the LFR1 as set forth in SEQ ID NO: 172, the LFR2 as set forth in SEQ ID NO:183, the LFR3 as set forth in SEQ ID NO:192, and the LFR4 as set forth in SEQ ID NO:61.

In some embodiments, the VL comprises the LFR1 as set forth in SEQ ID NO: 174, the LFR2 as set forth in SEQ ID NO:184, the LFR3 as set forth in SEQ ID NO:57, and the LFR4 as set forth in SEQ ID NO:199.

In some embodiments, the VL comprises the LFR1 as set forth in SEQ ID NO: 175, the LFR2 as set forth in SEQ ID NO: 185, the LFR3 as set forth in SEQ ID NO: 193, and the LFR4 as set forth in SEQ ID NO: 199.

In some embodiments, the VL comprises the LFR1 as set forth in SEQ ID NO: 176, the LFR2 as set forth in SEQ ID NO: 186, the LFR3 as set forth in SEQ ID NO: 194, and the LFR4 as set forth in SEQ ID NO: 199.

In some embodiments, the VL comprises the LFR1 as set forth in SEQ ID NO: 177, the LFR2 as set forth in SEQ ID NO: 187, the LFR3 as set forth in SEQ ID NO: 195, and the LFR4 as set forth in SEQ ID NO: 199.

In some embodiments, the VL comprises the LFR1 as set forth in SEQ ID NO: 178, the LFR2 as set forth in SEQ ID NO: 188, the LFR3 as set forth in SEQ ID NO: 196, and the LFR4 as set forth in SEQ ID NO: 199.

In some embodiments, the VL comprises the LFR1 as set forth in SEQ ID NO: 179, the LFR2 as set forth in SEQ ID NO: 187, the LFR3 as set forth in SEQ ID NO: 195, and the LFR4 as set forth in SEQ ID NO: 199.

In some embodiments, the VL comprises the LFR1 as set forth in SEQ ID NO: 173, the LFR2 as set forth in SEQ ID NO:183, the LFR3 as set forth in SEQ ID NO:192, and the LFR4 as set forth in SEQ ID NO:201.

In some embodiments, the VH comprises a sequence as set forth in any one of SEQ ID NOs: 202-220, 332-334.

In some embodiments, the VL comprises a sequences as set forth in any one of SEQ ID NOs: 221-234, 335-338.

In some embodiments, the isolated antigen-binding protein comprises a heavy chain constant region of IgG and/or the light chain constant region of human antibodies.

In some embodiments, the isolated antigen-binding protein comprising a heavy chain constant region of IgG1, IgG2, IgG3, or IgG4.

In some embodiments, the isolated antigen-binding protein comprising Kappa or Lambda light chain constant regions.

In some embodiments, Fc of the isolated antigen-binding protein is derived from IgG, IgM, IgA, IgD, or IgE.

In another aspect, the present application provides an isolated nucleic acid molecule encoding the previously isolated antigen-binding protein.

In another aspect, the present application provides an expression vector which comprises the aforementioned nucleic acids.

In another aspect, the present application provides a host cell comprising the aforementioned isolated nucleic acids and/or the expression vector.

In another aspect, the present application provides a composition comprising the aforementioned isolated antigen-binding protein and optionally a pharmaceutically acceptable carrier.

In another aspect, the application provides a method for preparing isolated antigen-binding protein TL1A, comprising:

culturing the host cells claimed above, and recovering the isolated antigen-binding protein.

In another aspect, the present application provides a method for the treatment of TL1A-mediated disease or conditions, which comprises: administration of the aforementioned isolated antigen-binding protein, aforementioned isolated nucleic acids, and/or aforementioned expression vectors, aforementioned host cells, or aforementioned compositions.

In another aspect, the present application provides antigen-binding protein isolated as described above, nucleic acids isolated as described above, and/or expression vectors described above, host cells as described above, or combinations described above, which are used for the treatment of TL1A-mediated diseases or conditions.

In another aspect, the present application provides the use of the aforementioned isolated antigen-binding protein, aforementioned isolated nucleic acids, and/or aforementioned expression vectors, aforementioned host cells, or aforementioned compositions in the treatment of TL1A-mediated diseases or conditions.

The aforementioned isolated antigen-binding proteins, the aforementioned isolated nucleic acids, and/or the aforementioned expression vectors, aforementioned host cells, or the aforementioned combinations are used in the preparation of drugs for the treatment of TL1A-mediated diseases or conditions.

In some embodiments, the disease or condition mediated by TL1A is inflammatory disease.

In some embodiments, TL1A-mediated inflammatory disease is selected from one of the following: allergy, ankylosing spondylitis, asthma, atopic dermatitis, autoimmune diseases or disorders, cancer, celiac disease, chronic obstructive pulmonary disease (COPD), chronic peptic ulcer, cystic fibrosis, diabetes (e.g., type 1 diabetes and type 2 diabetes), glomerulonephritis, gout, hepatitis (e.g., active hepatitis), an immune-mediated disease or disorder, inflammatory bowel disease (IBD) such as Crohn's disease and ulcerative colitis, myositis, osteoarthritis, pelvic inflammatory disease (PID), multiple sclerosis, neurodegenerative diseases of aging, periodontal disease (e.g., periodontitis), preperfusion injury transplant rejection, psoriasis, pulmonary fibrosis (e.g., idiopathic pulmonary fibrosis), rheumatic disease, scleroderma, sinusitis, tuberculosis.

In some embodiments, the TL1A-mediated disease or condition is an autoimmune disease. In some embodiments, TL1A-mediated autoimmune disease is selected from one of the following: Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal MotorNeuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

In some embodiments, the disease or condition mediated by TL1A is cancer.

In some embodiments, TL1A-mediated cancer is selected from one or more of the following: Adenoid Cystic Carcinoma, Adrenal Gland Cancer, Amyloidosis, Anal Cancer, Ataxia-Telangiectasia, Atypical Mole Syndrome, Basal Cell Carcinoma, Bile Duct Cancer, Birt Hogg Dube Syndrome, Bladder Cancer, Bone Cancer, Brain Tumor, Breast Cancer, Breast Cancer in Men, Carcinoid Tumor, Cervical Cancer, Colorectal Cancer, Ductal Carcinoma, Endometrial Cancer, Esophageal Cancer, Gastric Cancer, Gastrointestinal Stromal Tumor (GIST), HER2-Positive Breast Cancer, Islet Cell Tumor, Juvenile Polyposis Syndrome, Kidney Cancer, Laryngeal Cancer, Leukemia-Acute Lymphoblastic Leukemia, Leukemia-Acute Lymphocytic (ALL), Leukemia-Acute Myeloid AML, Leukemia-Adult, Leukemia-Childhood, Leukemia-Chronic Lymphocytic (CLL), Leukemia-Chronic Myeloid (CIVIL), Liver Cancer, Lobular Carcinoma, Lung Cancer, Lung Cancer-Small Cell (SCLC), Lung Cancer-Non-small Cell (NSCLC), Lymphoma-Hodgkin's, Lymphoma-Non-Hodgkin's, Malignant Glioma, Melanoma, Meningioma, Multiple Myeloma, Myelodysplastic Syndrome (MDS), Nasopharyngeal Cancer, Neuroendocrine Tumor, Oral Cancer, Osteosarcoma, Ovarian Cancer, Pancreatic Cancer, Pancreatic Neuroendocrine Tumors, Parathyroid Cancer, Penile Cancer, Peritoneal Cancer, Peutz-Jeghers Syndrome, Pituitary Gland Tumor, Polycythemia Vera, Prostate Cancer, Renal Cell Carcinoma, Retinoblastoma, Salivary Gland Cancer, Sarcoma, Sarcoma-Kaposi, Skin Cancer, Small Intestine Cancer, Stomach Cancer, Testicular Cancer, Thymoma, Thyroid Cancer, Uterine (Endometrial) Cancer, Vaginal Cancer, and Wilms' Tumor.

Other aspects and advantages of the present application will become readily apparent to those skilled in the art from the following detailed description. Only exemplary embodiments of the present application are shown and described in the following detailed description. As will be recognized by those skilled in the art, the contents of the present application enable those skilled in the art to make modifications to the specific embodiments disclosed without departing from the spirit and scope of the present application to which the present application pertains. Accordingly, the drawings and description herein are to be regarded as illustrative in nature, and not as restrictive.

### BRIEF DESCRIPTION OF THE DRAWINGS

Specific features of the present application to which the present application relates are as set forth in the appended claims. The features and advantages of the present application to which the present application relates will be better understood by reference to the exemplary embodiments and drawings described in detail below. A brief description of the drawings is as follows:

FIG. 1A shows the predicted structure of different anti-TL1A antibodies binding with TL1A antigen as described in the present application (top view):

FIG. 1B shows the predicted structure of different anti-TL1A antibodies binding with TL1A antigen as described in the present application (side view)

FIGS. 2A-2D show the binding curves of different anti-TL1A antibodies described in the present application to human TL1A-his monomeric protein, and the curve from top to bottom indicates that the concentration is from high to low;

FIGS. 3A-3D show the binding curves of different anti-TL1A antibodies as described in the present application to human TL1A-his trimeric protein, and the curve from top to bottom indicates that the concentration is from high to low;

FIGS. 4A-4D show different anti-TL1A antibody binding curves to monkey TL1A-his protein as described in the present application, and the curve from top to bottom indicates that the concentration is from high to low;

FIGS. 5A-5B show binding curves of different anti-TL1A antibodies described in the present application to rat TL1A-his protein, and the curve from top to bottom indicates that the concentration is from high to low;

FIG. 6A showing the results of an ELISA assay for the binding ability of anti-TL1A antibodies described in the present application to human TL1A-his (trimer);

FIG. 6B showing the results of the ELISA assay detecting the binding capacity of the anti-TL1A antibody described in the present application to murine TL1A-his protein;

FIG. 6C shows the results of the ELISA assay for the binding capacity assay of the anti-TL1A antibody described in the present application to the monkey TL1A-his protein:

FIG. 6D shows the results of the ELISA assay for the binding capacity assay of the anti-TL1A antibody described in the present application to rat TL1A-his protein:

FIG. 7 shows the binding capacity of the anti-TL1A antibody described in the present application to CHO-K1-huTL1A cells:

FIG. 8 shows the anti-TL1A antibody described in the present application blocking TL1A-his protein binding to 293-DR3 cells:

FIG. 9 shows the blocking activity of anti-TLA0015 humanized antibodies described in this application:

FIG. 10 shows the TLA0238 humanized antibody blocking activity as described in this application:

FIGS. 11A-11D show the inhibition of soluble TL1A-activated NF-κB signaling pathway by the anti-TL1A antibodies described in this application:

FIG. 12 shows the inhibition of the membrane-bound TL1A-activated NF-κB signaling pathway by the anti-TL1A antibodies described in this application:

FIG. 13 shows the inhibition of TL1A-activated Caspase3/7 activity by an anti-TL1A antibody as described in the present application:

FIGS. 14A-14B show the inhibition of TL1A-activated Caspase3/7 activity by the TLA0015 humanized antibodies described in this application;

FIG. 15A shows that the anti-TL1A antibody described in this application inhibits IFN-γ secretion by T cells (Donor 1);

FIG. 15B shows an anti-TL1A antibody as described in the present application inhibiting IFN-γ secretion by T cells (Donor 2);

FIG. 15C shows an anti-TL1A antibody as described in the present application inhibiting IFN-γ secretion by T cells (Donor 3);

FIG. 15D shows an anti-TL1A antibody inhibiting IFN-γ secretion by T cells as described in the present application (Donor 4):

FIGS. 16A-16C shows an anti-TL1A antibody as described in the present application inhibiting TNF-α secretion by T cells (Donor 5-7):

FIG. 17 shows inhibition of TNF-α secretion by T cells by an anti-TL1A antibody as described in the present application:

FIG. 18 shows the ADCC activity of the TLA0015-11 antibody described in the present application;

FIG. 19 shows the TLA0015-11 antibody CDC activity as described in the present application: FIGS. 20A-20F show the effect of the TLA0015-11 antibodies described in this application on cytokine release:

FIGS. 21A-21K show the affinity results of the TLA0015-11 antibody described in the present application to different Fcγ receptors, complement C1q, and FcRn proteins:

FIG. 22 shows the anti-TL1A antibody described in this application in PK assay in B-hFcRn transgenic mice:

FIG. 23 shows the anti-TL1A antibody described in this application PK assay in B-hFcRn transgenic mice:

FIG. 24 shows the effect of anti-TL1A antibodies described in the present application on body weight changes in B-hTL1A humanized transgenic mice in the DSS-induced enteritis model:

FIG. 25 shows the effect of anti-TL1A antibodies described in the present application on colorectal length in B-hTL1A humanized transgenic mice in a DSS-induced enteritis model:

FIGS. 26A-26B show the effect of the anti-TL1A antibodies described in the present application on the change in body weight of B-hTL1A humanized transgenic mice in a DSS-induced enteritis model:

FIG. 27 shows the results of the anti-TL1A antibody described in the present application for daily scoring of B-hTL1A humanized transgenic mice in a DSS-induced enterocolitis model:

FIGS. 28A-28B show the effect of anti-TL1A antibodies described in the present application on colorectal length in B-hTL1A humanized transgenic mice in a DSS-induced enteritis model:

FIGS. 29A-29B show the effect of anti-TL1A antibodies described in the present application on colorectal weight in B-hTL1A humanized transgenic mice in a DSS-induced enteritis model:

FIGS. 30A-30B show the effect of the anti-TL1A antibodies described in the present application on the change in body weight of B-hTL1A humanized transgenic mice in a TNBS-induced enteritis model:

FIG. 31 shows the results of the anti-TL1A antibody described in the present application for daily scoring of B-hTL1A humanized transgenic mice in a TNBS-induced enterocolitis model:

FIG. 32 shows the effect of the anti-TL1A antibodies described in the present application on the survival curves of B-hTL1A humanized transgenic mice in a TNBS-induced enteritis model:

FIGS. 33A-33B show the effect of the anti-TL1A antibodies described in the present application on colorectal length in B-hTL1A humanized transgenic mice in a TNBS-induced enteritis model:

FIGS. 34A-34B show the effect of anti-TL1A antibodies described in the present application on colorectal weight in B-hTL1A humanized transgenic mice in a TNBS-induced enteritis model:

FIGS. 35A-35B show the effect of anti-TL1A antibodies described in the present application on the change in body weight of B-hTL1A humanized transgenic mice in a TNBS-induced enteritis model:

FIG. 36 shows the results of the anti-TL1A antibody described in the present application for daily scoring of B-hTL1A humanized transgenic mice in a TNBS-induced enterocolitis model:

FIG. 37 shows the effect of the anti-TL1A antibodies described in the present application on the survival curves of B-hTL1A humanized transgenic mice in a TNBS-induced enteritis model:

FIGS. 38A-38B show the effect of the anti-TL1A antibodies described in the present application on colorectal length in B-hTL1A humanized transgenic mice in a TNBS-induced enteritis model:

FIGS. 39A-39B show the effect of anti-TL1A antibodies described in the present application on colorectal weight in B-hTL1A humanized transgenic mice in a TNBS-induced enteritis model.

### DETAILED DESCRIPTION OF THE INVENTION

The embodiments of the invention of the present application are illustrated by particular examples. Other advantages and effects of the present application will become readily apparent to those skilled in the art from disclosure of the present specification.

### Definition of Terms

In the present application, the term “isolated” generally refers to a product obtained from a natural state by artificial means. If an “isolated” substance or component occurs in nature, it may be altered from its natural environment, or the substance may be isolated from its natural environment, or both. For example, an unisolated polynucleotide or polypeptide naturally occurs in a living animal and the same polynucleotide or polypeptide isolated from its natural state in high purity is the to be isolated. The term “isolated” does not exclude the admixture of artificial or synthetic substances, nor the presence of other impure substances which do not affect the activity of the substance.

In the present application, the term “antigen-binding protein” generally refers to a polypeptide molecule capable of specifically recognizing and/or neutralizing a particular antigen. In the present application, the term “antigen-binding protein” may include an “antibody” or an “antigen-binding fragment”. For example, the antibody may comprise an immunoglobulin composed of at least two heavy (H) chains and two light (L) chains interconnected by disulfide bonds, and may include any molecule comprising an antigen-binding portion thereof. The term “antibody” may include monoclonal antibodies, antibody fragments, or antibody derivatives, including, but not limited to, murine antibodies, human antibodies (fully human antibodies), humanized antibodies, chimeric antibodies, single chain antibodies (e.g., scFv), and antibody fragments that bind to an antigen (e.g., Fab, Fab′, and (Fab)2 fragments). The term “antibody” may also include all recombinant forms of antibodies, such as antibodies expressed in prokaryotic cells, non-glycosylated antibodies, and any antigen-binding antibody fragments and derivatives thereof described herein. Each heavy chain may be composed of a heavy chain variable region and a heavy chain constant region. Each light chain may be composed of a light chain variable region and a light chain constant region. The VH and VL regions can be further distinguished as hypervariable regions called complementarity determining regions (CDRs), interspersed with more conserved regions called framework regions (FRs). Each VH and VL may be composed of three CDRs and four FRs, which may be arranged from amino-terminus to carboxy-terminus in the following order: FR1, CDR1, FR2, CDR2, FR3, CDR3, and FR4. The variable regions of the heavy and light chains contain binding domains that interact with an antigen. The constant region of an antibody can mediate the binding of the immunoglobulin to host tissues or factors, including various cells of the immune system (e.g., effector cells) and the first component of the classical complement system (C1q). The exact boundaries of the CDRs have been defined differently for different systems. The system described by Kabat (Kabat et al., Sequences of Proteins of Immunological Interest (National Institutes of Health, Bethesda, Md. (1987) and (1991)) provides not only an unambiguous residue numbering system applicable to any variable region of an antigen-binding fragment, but also provides precise residue boundaries defining CDRs. These CDRs may be referred to as Kabat CDRs. Chothia and co-workers (Chothia and Lesk, J. Mol. Biol. 196:901-917 (1987) and Chothia et al., Nature 342:877-883 (1989)) found that certain sub-portions within Kabat CDRs adopt nearly identical conformations in the backbone of a peptide despite the large diversity at the amino acid sequence level. These sub-portions are designated as L1, L2, and L3 or H1, H2, and H3, where “L” and “H” refer to the light chain and heavy chain regions, respectively. These regions may be referred to as Chothia CDRs, which have boundaries that overlap with Kabat CDRs. Other boundaries that define CDRs and overlap with Kabat CDRs have been described by Padlan (FASEB J. 9:133-139 (1995)) and MacCallum (J Mol Biol 262 (5): 732-45 (1996)). In addition, other CDR boundary definitions may not strictly follow one of the above systems, but will still overlap with Kabat CDRs, although they may be shortened or lengthened in light of prediction or experimental findings that a particular residue or group of residues or even the entire CDRs, do not significantly affect the antigen binding. In the present application, the Kabat numbering system is used.

In the present application, the term “antigen-binding fragment” generally refers to one or more fragments of an antibody that function to specifically bind to an antigen. The antigen-binding function of an antibody can be achieved by a full-length fragment of the antibody. The antigen-binding function of an antibody may also be achieved by: a heavy chain comprising a fragment of Fv, scFv, dsFv, Fab, Fab′ or F(ab′)2, or a light chain comprising a fragment of Fv, scFv, dsFv, Fab, Fab′, or F(ab′)2. (1) An Fab fragment, i.e., a monovalent fragment consisting of the VL, VH, CL, and CH domains: (2) an F(ab′) 2 fragment, a bivalent fragment comprising two Fab fragments linked by a disulfide bond at the hinge region: (3) an Fd fragment consisting of the VH and CH domains: (4) an Fv fragment consisting of the VL and VH domains of a single arm of an antibody: (5) a dAb fragment consisting of VH domains (Ward et al., (1989) Nature 341:544-546): (6) an isolated complementarity determining region (CDR), and (7) a combination of two or more isolated CDRs which may optionally be joined by a linker. For example, monovalent single chain molecules Fv (scFv) formed by the pairing of VL and VH may also be included (see, Bird et al., (1988) Science 242:423-426; and Huston et al., (1988) Proc. Natl. Acad. Sci. 85:5879-5883). For example, a class of antibody VHH lacking the antibody light chain but only the heavy chain variable region can also be included (see, e.g., Kang Xiaozhen et al., Chinese Journal of Biotechnology, 2018, 34 (12): 1974-1984). The “antigen-binding fragment” may also include an immunoglobulin fusion protein comprising a binding domain selected from the group consisting of: (1) a binding domain polypeptide fused to an immunoglobulin hinge region polypeptide: (2) an immunoglobulin heavy chain CH2 constant region fused to the hinge region; and (3) an immunoglobulin heavy chain CH3 constant region fused to the CH2 constant region.

In the present application, the term “monoclonal antibody” generally refers to a population of substantially homologous antibodies, i.e., the individual antibodies comprising the population are identical except for possible naturally occurring mutations that may be present in minor amounts. Monoclonal antibodies are highly specific, being directed against a single antigenic site. For example, the monoclonal antibodies can be prepared by hybridoma techniques or can be produced in bacteria, eukaryotic animal or plant cells using recombinant DNA methods, or can be derived from phage antibody libraries using the techniques, for example, described in Clackson et al., Nature, 352:624-628 (1991) and Marks et al., Mol. Biol., 222:581-597 (1991).

In the present application, the term “chimeric antibody” generally refers to an antibody in which a portion of the amino acid sequence of each heavy or light chain is homologous to the corresponding amino acid sequence in an antibody from a particular species, or belongs to a particular class, while the remaining segments of the chain are homologous to the corresponding sequence in another species. For example, the variable regions of both the light and heavy chains are derived from the variable region of an antibody of one animal species (e.g., mouse, rat, etc.), while the constant portions are homologous to antibody sequences from another species (e.g., human). For example, to obtain chimeric antibodies, non-human-derived B cells or hybridoma cells can be used to generate variable regions, while which the constant regions in combination therewith are of human origin. The variable region has the advantage of being easy to prepare and its specificity is not influenced by the origin of the constant region with which it is combined. Also, since the constant region of the chimeric antibody can be derived from human, the chimeric antibodies are less likely to elicit an immune response upon injection than using antibodies whose constant regions are of non-human origin.

In the present application, the term “humanized antibody” generally refers to a chimeric antibody that comprises fewer sequences from non-human immunoglobulins, thereby reducing the immunogenicity of a xenogenous antibody when introduced into humans, while maintaining the full antigen-binding affinity and specificity of the antibody. For example, non-human binding domains can be humanized using the technical means, such as CDR transplantion (Jones et al., Nature 321: 522 (1986)) and variants thereof: including “reshaping”, (Verhoeyen, et al., 1988 Science 239:1534-1536: Riechmann, et al., 1988 Nature 332:323-337: Tempest, et al., Bio/Technol 1991 9:266-271), “hyperchimerization” (Queen, et al., 1989 Proc Natl Acad Sci USA 86:10029-10033: Co, et al., 1991 Proc Natl Acad Sci USA 88:2869-2873: Co, et al., 1992 J Immunol 148:1149-1154), and “veneering” (Mark, et al., “Derivation of therapeutically active humanized and veneered anti-CD18 antibodies.” In: Metcalf B W, Dalton B J, eds. Cellular adhesion: molecular definition to therapeutic potential. New York: Plenum Press, 1994:291-312), and resurfacing (U.S. Pat. No. 5,639,641). Other regions, such as hinge and constant region domains, may also be humanized if they are also derived from non-human sources.

In the present application, the term “mouse antibody” generally refers to an antibody whose variable region framework and CDR region are derived from mouse germline immunoglobulin sequences. In addition, if the antibody comprises a constant region, it is also derived from mouse germline immunoglobulin sequences. The mouse antibody of the present application may comprise amino acid residues not encoded by mouse germline immunoglobulin sequences, for example, may comprise mutations introduced by in vitro random mutations or point mutations or by in vivo somatic mutations.

In this application, the term “TL1A” refers to TNF-like protein 1A. TL1A (TNFSF15) is a member of the TNF family that is expressed predominantly by endothelial cells, macrophages and dendritic cells (DCs). Its expression is induced by immune complexes (ICs) and cytokines. TL1A receptor DR3 is expressed mainly on T cells and NKT cells. In vitro, TL1A has been shown to enhance T cell proliferation and cytokine production in both humans and mice. In vivo, TL1A transgenic mice produce an IBD phenotype similar to human Crohn's disease.

In addition to the specific proteins and nucleotides mentioned herein, the present application may also include functional variants, derivatives, analogs, homologs, and fragments thereof.

The term “functional variant” refers to an amino acid sequence that is substantially identical to a naturally occurring sequence or a polypeptide encoded by a substantially identical nucleotide sequence and capable of having one or more activities of the naturally occurring sequence. In the context of the present application, a variant of any given sequence refers to a sequence in which the particular sequence of residues, whether amino acid or nucleotide residues, has been modified such that the polypeptide or polynucleotide substantially retains at least one endogenous function. Variant sequences may be obtained by the addition, deletion, substitution, modification, substitution and/or variation of at least one amino acid residue and/or nucleotide residue present in a naturally occurring protein and/or polynucleotide, so long as the original functional activity is retained.

In the present application, the term “derivative” generally refers to a polypeptide or polynucleotide of the present application comprising any substitution, variation, modification, replacement, deletion and/or addition of one (or more) amino acid residues from/on the sequence, so long as the resulting polypeptide or polynucleotide substantially retains at least one of its endogenous functions.

In the present application, the term “analog” generally refers to a polypeptide or polynucleotide including any mimetic of a polypeptide or polynucleotide, i.e., a chemical compound that possesses at least one endogenous function of the polypeptide or polynucleotide that the mimetic mimics.

Generally, amino acid substitutions, e.g., at least one (e.g., 1, 2, 3, 4, 5, 6, 7, 8, 9, 10, or 20 or more) amino acid substitution can be made, so long as the modified sequence substantially retains the desired activity or ability. Amino acid substitutions can include the use of non-naturally occurring analogs.

In the present application, the term “homologue” generally refers to an amino acid sequence or nucleotide sequence having certain homology to a naturally occurring sequence. The term “homology” can be equivalent to sequence “identity”. Homologous sequences may include amino acid sequences that may be at least 80%, 85%, 90%, 99.1%, 99.2%, 99.3%, 99.4%, 99.5%, 99.6%, 99.7%, 99.8%, or 99.9% identical to the subject sequence. Typically, the homologue will comprise the same active site or the like as the subject amino acid sequence. Homology may be considered in terms of similarity (i.e., amino acid residues having similar chemical properties/functions) or may be expressed in terms of sequence identity. In the present application, a sequence having percent identity in any one of the SEQ ID NO of a mentioned amino acid sequence or nucleotide sequence refers to a sequence having the percent identity over the entire length of the mentioned SEQ ID NO. To determine sequence identity, sequence alignments may be performed by a variety of ways known to those skilled in the art, e.g., using BLAST, BLAST-2, ALIGN, NEEDLE, or Megalign (DNASTAR) software, etc. Those skilled in the art can determine the appropriate parameters for the alignment, including any algorithm required to achieve the optimal alignment over the full length of the sequence being compared.

The proteins or polypeptides used in the present application may also have deletions, insertions, or substitutions of amino acid residues which produce silent changes and result in functionally equivalent proteins. Intentional amino acid substitutions may be made on the basis of similarity in polarity, charge, solubility, hydrophobicity, hydrophilicity, and/or amphipathic nature of the residues, so long as the endogenous function is retained. For example, negatively charged amino acids include aspartic acid and glutamic acid: positively charged amino acids include lysine and arginine; and amino acids containing uncharged polar head groups with similar hydrophilicity values include asparagine, glutamine, serine, threonine, and tyrosine.

In the present application, the term “immunoconjugate” generally refers to a conjugate formed by the conjugation of other agents (e.g., a chemotherapeutic agent, a radioactive element, a cytostatic agent, and a cytotoxic agent) to the isolated antigen-binding protein (e.g., via covalent attachment of a linking molecule), wherein the conjugate can deliver the other agents to a target cell (e.g., a tumor cell) via specific binding of the isolated antigen-binding protein to an antigen on the target cell. The immunoconjugate then undergoes such internalization and eventually enters the interior of the target cell (e.g., into vesicles such as a lysosome), at which point the linker molecule in the immunoconjugate can be cleaved to release the other agent, thereby exerting its cytotoxic effect. In addition, the antigen may also be secreted by the target cell and located in the space outside the target cell.

In the present application, the term “subject” generally refers to human or non-human animals, including, but not limited to, cats, dogs, horses, pigs, cows, caprid, rabbits, mice, rats, or monkeys.

In the present application, the term “nucleic acid molecule” generally refers to an isolated form of nucleotides, deoxyribonucleotides, or ribonucleotides or analogs thereof, of any length, isolated from their natural environment or artificially synthesized.

In the present application, the term “vector” generally refers to a nucleic acid molecule capable of self-replication in a suitable host. The vector can transfer the inserted nucleic acid molecule into and/or between cells. The vector may include a vector for primarily inserting DNA or RNA into a cell, a vector for primarily replicating DNA or RNA, and a vector for primarily expressing transcription and/or translation of DNA or RNA. The vector can be a polynucleotide capable of being transcribed and translated into a polypeptide when introduced into an appropriate cell. In general, the vector may produce the desired expression product by culturing an appropriate cell containing the vector. In the present application, the vector may include a lentiviral vector.

In the present application, the term “cell” generally refers to an individual cell, cell line or cell culture that may or may already contain a plasmid or vector comprising a nucleic acid molecule as described herein, or that is capable of expressing a polypeptide as described herein or an antigen-binding protein as described herein. The cell may include the progeny of a single cell. Due to natural, accidental, or deliberate mutations, the progeny cells may not necessarily be identical in morphology or in genome to the original parent cell, but are capable of expressing the polypeptide or antigen-binding protein as described herein. The cells can be obtained by transfecting cells in vitro with the vectors as described herein. The cells may be prokaryotic cells (e.g., E. coli) or eukaryotic cells (e.g., yeast cell, COS cells, Chinese Hamster Ovary (CHO) cells, HeLa cells, HEK293 cells, COS-1 cells, NS0 cells, or myeloma cells). In some embodiments, the cells may be immune cells. For example, the immune cell may be selected from the group consisting of T cells, B cells, natural killer cells (NK cells), macrophages, NKT cells, monocytes, dendritic cells, granulocytes, lymphocytes, leukocytes and/or peripheral blood mononuclear cells.

In the present application, the term “treatment” generally refers to: (i) the prevention of the development of a disease, disorder, and/or condition in a patient who may be susceptible to, but has not yet been diagnosed with, that disease, disorder, or condition: (ii) the suppression of the disease, disorder, or condition, i.e., the curb of the development; and (iii) remission of the disease, disorder, or condition, i.e., causing regression of the disease, disorder, and/or condition and/or symptoms associated with the disease, disorder, and/or condition.

In the present application, the terms “polypeptide”, “peptide”, and “protein” are used interchangeably and generally refer to a polymer of amino acids of any length. The polymer may be linear or branched, and may comprise modified amino acids and it may be interrupted by non-amino acids. These terms also encompass amino acid polymers that have been modified. These modifications may comprise: disulfide bond formation, glycosylation, lipidation, acetylation, phosphorylation, or any other manipulation (e.g., binding to a labeling component). The term “amino acid” comprises natural and/or non-natural or synthetic amino acids, including glycine as well as the D and L optical isomers, as well as amino acid analogs and peptidomimetics.

In the present application, the terms “polynucleotide”, “nucleotide”, “nucleotide sequence”, “nucleic acid”, and “oligonucleotide” are used interchangeably and refer generally to polymeric forms of nucleotides of any length, such as deoxyribonucleotides or ribonucleotides, or analogs thereof. A polynucleotide may have any three-dimensional structure and may perform any function that is known or unknown. Non-limiting examples of polynucleotides are as follows: a coding or noncoding region of a gene or gene fragment, a plurality of loci (one locus) as defined by ligation analysis, exons, introns, messenger RNA (mRNA), transport RNA, ribosomal RNA, short interfering RNA (siRNA), short hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modification of the nucleotide structure may be performed before or after assembly of the polymer. The sequence of nucleotides may be interrupted by non-nucleotide components. The polynucleotides may be further modified after polymerization, e.g., by conjugation to labeled components.

In the present application, the term “K_{D}” (likewise, “K_{D}” or “KD”) generally refers to an “affinity constant” or an “equilibrium dissociation constant” and refers to a value obtained at equilibrium in a titration measurement, or by dividing the dissociation rate constant (k_{d}) by the binding rate constant (kₐ). The binding affinity of a binding protein (e.g., an isolated antigen-binding protein as described herein) for an antigen is expressed using an association rate constant (kₐ), a dissociation rate constant (k_{d}), and an equilibrium dissociation constant (K_{D}). Methods for determining association and dissociation rate constants are well known in the art. The use of fluorescence-based techniques provides high sensitivity and the ability to examine samples at equilibrium in physiological buffers. For example, the K_{D} value can be determined by Octet assay, and other experimental approaches and instruments such as BIAcore (Biomolecular Interaction Analysis) can be used (e.g., instruments available from BIAcoreInternationalAB, aGEHealthcarecompany, Uppsala, Sweden). Alternatively, the K_{D} value can be determined using KinExA (dynamic exclusion assay (KineticExclusionAssay)) available from SapidyneInstruments (Boise, Idaho) or using a surface plasmon resonance (SPR) instrument.

In the present application, the term “IC50 value” or the term “half-maximal inhibitory concentration” (IC50) indicates the concentration of a particular compound that is required to achieve 50% inhibition of a biological process in vitro. The IC50 value can be logarithmically converted to a pIC50 value (−log IC50), where higher values indicate exponentially greater potency. The IC50 value is not an absolute value, but rather depends on experimental conditions, such as the concentration used. The IC50 value can be converted to an absolute inhibitory constant (Ki) using the Cheng-Prusoff equation (Biochem. Pharmacol. (1973) 22:3099).

The term “EC50”, in the context of in vitro or in vivo analyses using isolated antigen-binding fragments, refers to the concentration of the antibody or antigen-binding portion thereof when the induced response is 50% of the maximal response, i.e., halfway between the maximal response and the baseline.

In the present application, the term “and/or” should be understood as meaning any one of the alternatives or both of the alternatives.

In the present application, the term “comprising” or “containing” generally refers to the inclusion of explicitly specified features, but not excluding other elements. In certain instances, “comprising” or “contain” also encompasses the inclusion of only the specified components.

In the present application, the term “about” generally refers to a range from 0.5% to 10% above or below the specified value, for example, a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% above or below the specified value.

### DETAILED DESCRIPTION OF THE INVENTION

### Isolated Antigen-Binding Protein

In one aspect, the present application provides an isolated antigen-binding protein that can bind to a monomer TL1A and/or trimer TL1A with a K_{D} value of about 1.0E-8M or less (For example, said K_{D} is no greater than about 5E-08M, no greater than about 4.5E-08M, no greater than about 4E-08M, no greater than about 3.5E-08M, no greater than about 3E-08M, no greater than about 2E-08M, no greater than about 2E-08M, no greater than about 1.5E-08M, no greater than about 1E-08M, no greater than about 5E-09M, no greater than about 4.5E-09M, no greater than about 4E-09M, no greater than about 3.5E-09M, no greater than about 3E-09M, no greater than about 2.5E-09M, no greater than about 2E-09M, no greater than about 1.5E-09M, no greater than about 1E-09M, no greater than about 9E-10M, no greater than about 5E-10M, no greater than about 1E-10M, no greater than about 5E-11M, no greater than about 1E-11M, or no greater than 5E-12M or less.)

In the present application, the isolated antigen-binding protein of the present disclosure can be detected in a flow-through assay at an EC50 value of about 20 nM or less (e.g., said EC50 value is no higher than about 20 nM, no higher than about 15 nM, no higher than about 10 nM, no higher than about 5 nM, no higher than about 2.5 nM, no higher than about 2 nM, no higher than about 1 nM, no higher than about 0.5 nM, or less) in relation to the antigen-binding proteins expressed in theCHOK1 cells specifically binds to human TL1A expressed on CHOKI cells.

In the present application, the isolated antigen-binding protein of the present disclosure is an antagonist of the TL1A receptor, including, but not limited to, DR3 and TR6/DcR3. In the present application, the isolated antigen-binding protein of the present disclosure blocks the interaction of TL1A with death receptor 3 (“DR3”). In the present application, said isolated antigen-binding protein blocks monomeric TL1A-protein and/or trimeric TL1A in a flow-through assay at an IC50 value of about 50 nM or less (e.g., said IC50 value is no higher than about 50 nM, no higher than about 45 nM, no higher than about 40 nM, no higher than about 35 nM, no higher than about 30 nM, no higher than about 25 nM or less) binding to DR3 (DR3: UniprotKB NO. Q93038).

In the present application, the isolated antigen-binding proteins can inhibit the activity of the soluble TL1A-activated NF-κB signaling pathway. For example, the soluble TL1A-activated NF-κB signaling pathway can be tested for inhibition by a luciferase reporter gene system.

In the present application, said isolated antigen-binding protein can inhibit the activity of the membrane-bound TL1A-activated NF-κB signaling pathway. For example, whether the membrane-bound TL1A-activated NF-κB signaling pathway is inhibited can be detected by a luciferase reporter gene system.

In the present application, said isolated antigen-binding protein may function to inhibit Caspase3/7 activity. For example, the function of the Caspase3/7 activity can be tested for inhibition by a luciferase reporter gene system.

On the other hand, the present application provides an antigen-binding protein which competitively binds TL1A with the antigen-binding protein described herein.

### Isolated Antigen-Binding Protein-TLA-0015

In one aspect, the present disclosure provides an antigen-binding protein which specifically binds to TL1A and comprises a heavy chain variable region (VH). The VH comprises at least one heavy chain complementarity determining regions (HCDR). The VH may include at least one, two or three of HCDR1, HCDR2 or HCDR3. In some embodiments, HCDR3, HCDR2 and HCDR1 are sequences as set forth in SEQ ID NO: 14, 5 and 1 respectively.

HCDR3 of the antigen-binding protein may include amino acid sequence as set forth in SEQ ID NO: 14. For example, the sequence of HCDR3 of the antigen-binding protein may be defined by Kabat.

In some embodiments, HCDR3 may be GX1X2DAMDY (SEQ ID NO:14). X1 may be D or L or N, X2 may be For Y.

In some embodiments, HCDR3 may include an amino acid sequence as set forth in any one of SEQ ID NOs: 15-18.

HCDR2 of the antigen-binding protein may include amino acid sequence as set forth in SEQ ID NO: 5. For example, the sequence of HCDR2 of the antigen-binding protein may be defined by Kabat.

In some embodiments, HCDR2 may be X3IWGFGX4TX5YX6X7ALKS (SEQ ID NO:5). X3 may be R or V, X4 may be G or K, X5 may be D or H or N, X6 may be Nor Q, X7 may be A or P or S.

In some embodiments, HCDR2 may include an amino acid sequence as set forth in any one of SEQ ID NOs: 6-13.

HCDR1 of the antigen-binding protein may include amino acid sequence as set forth in SEQ ID NO: 1. For example, the sequence of HCDR1 of the antigen-binding protein may be defined by Kabat.

In some embodiments, HCDR1 may be X8YGVD (SEQ ID NO:1). X8 may be S or T or Y.

In some embodiments, HCDR1 may include an amino acid as set forth in any one of SEQ ID NO: 2-4.

In some embodiments, the VH comprises HFR1, and C-terminus of HFR1 is directly or indirectly linked to the N-terminus of HCDR1. For example, the sequence of HFR1 of the antigen-binding protein may be defined by Kabat. HFR1 comprises an amino acid as set forth in SEQ ID NO: 19.

In some embodiments, HFR1 is

### EX9QLX10ESGGGLX11QPGGSLRX12SCAVSSGFXx1LXx2 (SEQ ID NO:19). X9 may be I or V, X10 may be Lor V, X11 may be I or V, X12 may be I or L. Xx1 is D or S, Xx2 is I or K or S.

In some embodiments, HFR1 is selected from an amino acid sequence as set forth in any one of SEQ ID NOs: 20-28.

In some embodiments, VH comprises HFR2, and HFR2 is located between HCDR1 and HCDR2. For example, the sequence of HFR2 of the antigen-binding protein may be defined by Kabat. HFR2 comprises an amino acid sequence as set forth in SEQ ID NO:29.

In some embodiments, HFR2 is WVRQX13PGKGLEWX14G (SEQ ID NO:29).X13 may be A or S,X14 may be L or V.

In some embodiments, HFR2 is selected from an amino acid sequence as set forth in any one of SEQ ID NOs: 30-32.

In some embodiments, VH comprises HFR3, and HFR3 is located between HCDR2 and HCDR3. For example, the sequence of HFR3 of the antigen-binding protein may be defined by Kabat. HFR3 comprises an amino acid as set forth in SEQ ID NO:33.

In some embodiments, HFR3 is

RX15TISX16DNSKNTX17YLQMNSLRAEDTAVYYCAX18 (SEQ ID NO:33). X15 may be For L,X16 may be A or K or R or V, X17 may be Lor V, X18 may be R or S.

In some embodiments, HFR3 is selected from an amino acid sequence as set forth in any one of SEQ ID NOs: 34-40.

In some embodiments, VH comprises HFR4, and N-terminus of HFR4 is directly or indirectly linked to C-terminus of HCDR3. For example, the sequence of HFR4 of the antigen-binding protein may be defined by Kabat. HFR4 comprises an amino acid sequence as set forth in SEQ ID NO:41.

In some embodiments, the isolated antigen-binding protein further comprises a light chain variable region (VL), and VL comprises at least one light chain complementarity determining regions (LCDR). VL comprises LCDR1, LCDR2 and LCDR3, which is selected from an amino acid sequence as set forth in any one of SEQ ID NO:42, SEQ ID NO:43 and SEQ ID NO:44 accordingly.

In some embodiments, VL comprises LCDR3 including an amino acid sequence as set forth in SEQ ID NO:44. For example, the sequence of LCDR3 of the antigen-binding protein may be defined by Kabat.

In some embodiments, VL comprises LCDR2 including an amino acid sequence as set forth in SEQ ID NO:43. For example, the sequence of LCDR2 of the antigen-binding protein may be defined by Kabat.

In some embodiments, VL comprises LCDR1 including an amino acid sequence as set forth in SEQ ID NO:42. For example, the sequence of LCDR1 of the antigen-binding protein may be defined by Kabat.

In some embodiments, VL comprises the LFR1, and C-terminus of the LFR1 is directly or indirectly linked to N-terminus of LCDR1. For example, the sequence of the LFR1 of the antigen-binding protein may be defined by Kabat. The LFR1 comprises an amino acid sequence as set forth in SEQ ID NO:45.

In some embodiments, the LFR1 is DIQMTQX19X20SSLSASVGDRVTIX21C (SEQ ID NO: 45). X19 may be S or T, X20 may be Por T, X21 may be T or S.

In some embodiments, the LFR1 comprises an amino acid sequence as set forth in any one of SEQ ID NOs: 46-47.

In some embodiments, VL comprises the LFR2, and the LFR2 is located between LCDR1 and LCDR2. For example, the sequence of the LFR2 of the antigen-binding protein may be defined by Kabat, the LFR2 comprises an amino acid sequence as set forth in SEQ ID NO:48.

In some embodiments, the LFR2 is WYQQKPGKX22X23KLLIY (SEQ ID NO:48). X22 may be A or V,X23 may be Por V.

In some embodiments, the LFR2 comprises an amino acid sequence as set forth in any one of SEQ ID NOs: 49-52.

In some embodiments, VL comprises the LFR3, and the LFR3 is located between LCDR2 and LCDR3. For example, the sequence of the LFR3 of the antigen-binding protein may be defined by Kabat. The LFR3 comprises an amino acid sequence as set forth in SEQ ID NO:53.

In some embodiments, the LFR3 is

### GVPSRFSGSGSGTDX24TLTISSLQPEDX25ATYX26C (SEQ ID NO:53). X24 may be For Y. X25 may be For V. X26 may be For Y.

In some embodiments, the LFR3 comprises an amino acid sequence as set forth in any one of SEQ ID NOs: 54-57.

In some embodiments, the isolated antigen-binding protein comprises the LFR4, and N-terminus of the LFR4 is directly or indirectly linked to C terminus of LCDR3. For example, the sequence of the LFR4 of the antigen-binding protein may be defined by Kabat. The LFR4 comprises an amino acid sequence as set forth in SEQ ID NO:58.

In some embodiments, the LFR4 is FGX27GTKLEIK (SEQ ID NO:58). X27 may be Por Q.

In some embodiments, the LFR4 comprises an amino acid sequence as set forth in any one of SEQ ID NOs: 59-61.

In some embodiments, VH comprises sequences as set forth in SEQ ID NOs: 62-99.

In some embodiments, VL comprises sequences as set forth in SEQ ID NOs: 102-106.

In some embodiments, said isolated antigen-binding protein comprises a VH as shown in SEQ ID NO: 70 and a VL as shown in SEQ ID NO: 106.

In some embodiments, the VH of said isolated antigen-binding protein comprises an HCDR1 as shown in SEQ ID NO:2, an HCDR2 as shown in SEQ ID NO:6, and an HCDR3 as shown in SEQ ID NO: 15, and further comprises a light chain variable region (VL), said VL comprising at least one light chain complementary determining region (LCDR) comprising an LCDR1 shown in SEQ ID NO:42, an LCDR2 shown in SEQ ID NO:43 and an LCDR3 shown in SEQ ID NO:44.

In some embodiments, the antigen-binding proteins described above are mentioned in example 4, Tables 3 and 4.

In some embodiments, the distribution of antibodies for the TLA-0015 line is shown in the following table, with Table 1-1 showing the distribution of heavy chain components and Table 1-2 showing the distribution of light chain components. The technical solution of the present application includes, for the same antibody, a combination of HCDR1-3 in Table 1-1 and LCDR1-3 in Table 1-2. The technical solution of the present application includes, for the same antibody, a combination of VH in Table 1-1 and VL in Table 1-2.

TABLE 1-1

| | VH | | | | | | | | | | | | | |
|---|---|---|---|---|---|---|---|---|---|---|---|---|---|---|
| | | SEQ | | SEQ | | SEQ | | SEQ | | SEQ | | SEQ | | SEQ |
| antibody | HFR1 | ID | HCDR1 | ID | HFR2 | ID | HCDR2 | ID | HFR3 | ID | HCDR3 | ID | HFR4 | ID |
| TLA0015 | | | TLA0015- | 2 | TLA0015- | 32 | TLA0015- | 6 | | | TLA0015- | 15 | | |
| (mouse) | | | HCDR1-1 | | HFR2-3 | | HCDR2-1 | | | | HCDR3-1 | | | |
| TLA0015- | TLA0015- | 20 | TLA0015- | 2 | TLA0015- | 30 | TLA0015- | 6 | TLA0015- | 34 | TLA0015- | 15 | TLA0015- | 41 |
| 1 | HFR1-1 | | HCDR1-1 | | HFR2-1/ | | HCDR2-1 | | HFR3-1 | | HCDR3-1 | | HFR4-1/TLA- | |
| | | | | | | | | | | | | | 0238-HFR4-2 | |
| TLA0015- | TLA0015- | 20 | TLA0015- | 2 | TLA0015- | 30 | TLA0015- | 6 | TLA0015- | 34 | TLA0015- | 15 | TLA0015- | 41 |
| 2 | HFR1-1 | | HCDR1-1 | | HFR2-1/ | | HCDR2-1 | | HFR3-1 | | HCDR3-1 | | HFR4-1/TLA- | |
| | | | | | | | | | | | | | 0238-HFR4-2 | |
| TLA0015- | TLA0015- | 21 | TLA0015- | 2 | TLA0015- | 31 | TLA0015- | 6 | TLA0015- | 35 | TLA0015- | 15 | TLA0015- | 41 |
| 3 | HFR1-2 | | HCDR1-1 | | HFR2-2 | | HCDR2-1 | | HFR3-2 | | HCDR3-1 | | HFR4-1/TLA- | |
| | | | | | | | | | | | | | 0238-HFR4-2 | |
| TLA0015- | TLA0015- | 21 | TLA0015- | 2 | TLA0015- | 31 | TLA0015- | 6 | TLA0015- | 35 | TLA0015- | 15 | TLA0015- | 41 |
| 4 | HFR1-2 | | HCDR1-1 | | HFR2-2 | | HCDR2-1 | | HFR3-2 | | HCDR3-1 | | HFR4-1/TLA- | |
| | | | | | | | | | | | | | 0238-HFR4-2 | |
| TLA0015- | TLA0015- | 22 | TLA0015- | 2 | TLA0015- | 32 | TLA0015- | 6 | TLA0015- | 35 | TLA0015- | 15 | TLA0015- | 41 |
| 5 | HFR1-3 | | HCDR1-1 | | HFR2-3 | | HCDR2-1 | | HFR3-2 | | HCDR3-1 | | HFR4-1/TLA- | |
| | | | | | | | | | | | | | 0238-HFR4-2 | |
| TLA0015- | TLA0015- | 22 | TLA0015- | 2 | TLA0015- | 32 | TLA0015- | 6 | TLA0015- | 35 | TLA0015- | 15 | TLA0015- | 41 |
| 6 | HFR1-3 | | HCDR1-1 | | HFR2-3 | | HCDR2-1 | | HFR3-2 | | HCDR3-1 | | HFR4-1/TLA- | |
| | | | | | | | | | | | | | 0238-HFR4-2 | |
| TLA0015- | TLA0015- | 23 | TLA0015- | 2 | TLA0015- | 31 | TLA0015- | 6 | TLA0015- | 36 | TLA0015- | 15 | TLA0015- | 41 |
| 7 | HFR1-4 | | HCDR1-1 | | HFR2-2 | | HCDR2-1 | | HFR3-3 | | HCDR3-1 | | HFR4-1/TLA- | |
| | | | | | | | | | | | | | 0238-HFR4-2 | |
| TLA0015- | TLA0015- | 23 | TLA0015- | 2 | TLA0015- | 31 | TLA0015- | 6 | TLA0015- | 37 | TLA0015- | 15 | TLA0015- | 41 |
| 8 | HFR1-4 | | HCDR1-1 | | HFR2-2 | | HCDR2-1 | | HFR3-4 | | HCDR3-1 | | HFR4-1/TLA- | |
| | | | | | | | | | | | | | 0238-HFR4-2 | |
| TLA0015- | TLA0015- | 23 | TLA0015- | 2 | TLA0015- | 31 | TLA0015- | 6 | TLA0015- | 35 | TLA0015- | 15 | TLA0015- | 41 |
| 9 | HFR1-4 | | HCDR1-1 | | HFR2-2 | | HCDR2-1 | | HFR3-2 | | HCDR3-1 | | HFR4-1/TLA- | |
| | | | | | | | | | | | | | 0238-HFR4-2 | |
| TLA0015- | TLA0015- | 24 | TLA0015- | 2 | TLA0015- | 31 | TLA0015- | 6 | TLA0015- | 38 | TLA0015- | 15 | TLA0015- | 41 |
| 10 | HFR1-5 | | HCDR1-1 | | HFR2-2 | | HCDR2-1 | | HFR3-5 | | HCDR3-1 | | HFR4-1/TLA- | |
| | | | | | | | | | | | | | 0238-HFR4-2 | |
| TLA0015- | TLA0015- | 25 | TLA0015- | 2 | TLA0015- | 31 | TLA0015- | 6 | TLA0015- | 35 | TLA0015- | 15 | TLA0015- | 41 |
| 11 | HFR1-6 | | HCDR1-1 | | HFR2-2 | | HCDR2-1 | | HFR3-2 | | HCDR3-1 | | HFR4-1/TLA- | |
| | | | | | | | | | | | | | 0238-HFR4-2 | |
| TLA0015- | TLA0015- | 25 | TLA0015- | 2 | TLA0015- | 31 | TLA0015- | 6 | TLA0015- | 35 | TLA0015- | 15 | TLA0015- | 41 |
| 12 | HFR1-6 | | HCDR1-1 | | HFR2-2 | | HCDR2-1 | | HFR3-2 | | HCDR3-1 | | HFR4-1/TLA- | |
| | | | | | | | | | | | | | 0238-HFR4-2 | |
| TLA0015- | TLA0015- | 25 | TLA0015- | 2 | TLA0015- | 31 | TLA0015- | 6 | TLA0015- | 35 | TLA0015- | 15 | TLA0015- | 41 |
| 13 | HFR1-6 | | HCDR1-1 | | HFR2-2 | | HCDR2-1 | | HFR3-2 | | HCDR3-1 | | HFR4-1/TLA- | |
| | | | | | | | | | | | | | 0238-HFR4-2 | |
| TLA0015- | TLA0015- | 23 | TLA0015- | 2 | TLA0015- | 31 | TLA0015- | 6 | TLA0015- | 36 | TLA0015- | 15 | TLA0015- | 41 |
| 14 | HFR1-4 | | HCDR1-1 | | HFR2-2 | | HCDR2-1 | | HFR3-3 | | HCDR3-1 | | HFR4-1/TLA- | |
| | | | | | | | | | | | | | 0238-HFR4-2 | |
| TLA0015- | TLA0015- | 23 | TLA0015- | 2 | TLA0015- | 31 | TLA0015- | 6 | TLA0015- | 37 | TLA0015- | 15 | TLA0015- | 41 |
| 15 | HFR1-4 | | HCDR1-1 | | HFR2-2 | | HCDR2-1 | | HFR3-4 | | HCDR3-1 | | HFR4-1/TLA- | |
| | | | | | | | | | | | | | 0238-HFR4-2 | |
| TLA0015- | TLA0015- | 23 | TLA0015- | 2 | TLA0015- | 31 | TLA0015- | 6 | TLA0015- | 36 | TLA0015- | 15 | TLA0015- | 41 |
| 16 | HFR1-4 | | HCDR1-1 | | HFR2-2 | | HCDR2-1 | | HFR3-3 | | HCDR3-1 | | HFR4-1/TLA- | |
| | | | | | | | | | | | | | 0238-HFR4-2 | |
| TLa0015- | TLA0015- | 25 | TLA0015- | 2 | TLA0015- | 31 | TLA0015- | 6 | TLA0015- | 37 | TLA0015- | 15 | TLA0015- | 41 |
| 17 | HFR1-6 | | HCDR1-1 | | HFR2-2 | | HCDR2-1 | | HFR3-4 | | HCDR3-1 | | HFR4-1 | |
| TLa0015- | TLA0015- | 25 | TLA0015- | 2 | TLA0015- | 31 | TLA0015- | 6 | TLA0015- | 39 | TLA0015- | 15 | TLA0015- | 41 |
| 18 | HFR1-6 | | HCDR1-1 | | HFR2-2 | | HCDR2-1 | | HFR3-6 | | HCDR3-1 | | HFR4-1 | |
| TLa0015- | TLA0015- | 25 | TLA0015- | 2 | TLA0015- | 31 | TLA0015- | 7 | TLA0015- | 35 | TLA0015- | 15 | TLA0015- | 41 |
| 19 | HFR1-6 | | HCDR1-1 | | HFR2-2 | | HCDR2-2 | | HFR3-2 | | HCDR3-1 | | HFR4-1 | |
| TLa0015- | TLA0015- | 25 | TLA0015- | 2 | TLA0015- | 31 | TLA0015- | 8 | TLA0015- | 35 | TLA0015- | 15 | TLA0015- | 41 |
| 20 | HFR1-6 | | HCDR1-1 | | HFR2-2 | | HCDR2-3 | | HFR3-2 | | HCDR3-1 | | HFR4-1/TLA- | |
| | | | | | | | | | | | | | 0238-HFR4-2 | |
| TLa0015- | TLA0015- | 25 | TLA0015- | 2 | TLA0015- | 31 | TLA0015- | 6 | TLA0015- | 40 | TLA0015- | 15 | TLA0015- | 41 |
| 21 | HFR1-6 | | HCDR1-1 | | HFR2-2 | | HCDR2-1 | | HFR3-7 | | HCDR3-1 | | HFR4-1 | |
| TLA015- | TLA0015- | 25 | TLA0015- | 3 | TLA0015- | 31 | TLA0015- | 6 | TLA0015- | 35 | TLA0015- | 15 | TLA0015- | 41 |
| 22 | HFR1-6 | | HCDR1-2 | | HFR2-2 | | HCDR2-1 | | HFR3-2 | | HCDR3-1 | | HFR4-1 | |
| TLA015- | TLA0015- | 25 | TLA0015- | 2 | TLA0015- | 31 | TLA0015- | 6 | TLA0015- | 35 | TLA0015- | 16 | TLA0015- | 41 |
| 23 | HFR1-6 | | HCDR1-1 | | HFR2-2 | | HCDR2-1 | | HFR3-2 | | HCDR3-2 | | HFR4-1 | |
| TLA015- | TLA0015- | 25 | TLA0015- | 2 | TLA0015- | 31 | TLA0015- | 6 | TLA0015- | 35 | TLA0015- | 17 | TLA0015- | 41 |
| 24 | HFR1-6 | | HCDR1-1 | | HFR2-2 | | HCDR2-1 | | HFR3-2 | | HCDR3-3 | | HFR4-1 | |
| TLA015- | TLA0015- | 25 | TLA0015- | 2 | TLA0015- | 31 | TLA0015- | 6 | TLA0015- | 35 | TLA0015- | 18 | TLA0015- | 41 |
| 25 | HFR1-6 | | HCDR1-1 | | HFR2-2 | | HCDR2-1 | | HFR3-2 | | HCDR3-4 | | HFR4-1 | |
| TLA015- | TLA0015- | 25 | TLA0015- | 4 | TLA0015- | 31 | TLA0015- | 6 | TLA0015- | 35 | TLA0015- | 15 | TLA0015- | 41 |
| 26 | HFR1-6 | | HCDR1-3 | | HFR2-2 | | HCDR2-1 | | HFR3-2 | | HCDR3-1 | | HFR4-1 | |
| TLA015- | TLA0015- | 26 | TLA0015- | 2 | TLA0015- | 31 | TLA0015- | 6 | TLA0015- | 35 | TLA0015- | 15 | TLA0015- | 41 |
| 27 | HFR1-7 | | HCDR1-1 | | HFR2-2 | | HCDR2-1 | | HFR3-2 | | HCDR3-1 | | HFR4-1 | |
| TLA015- | TLA0015- | 25 | TLA0015- | 2 | TLA0015- | 31 | TLA0015- | 9 | TLA0015- | 35 | TLA0015- | 15 | TLA0015- | 41 |
| 28 | HFR1-6 | | HCDR1-1 | | HFR2-2 | | HCDR2-4 | | HFR3-2 | | HCDR3-1 | | HFR4-1 | |
| TLA015- | TLA0015- | 27 | TLA0015- | 2 | TLA0015- | 31 | TLA0015- | 6 | TLA0015- | 35 | TLA0015- | 15 | TLA0015- | 41 |
| 29 | HFR1-8 | | HCDR1-1 | | HFR2-2 | | HCDR2-1 | | HFR3-2 | | HCDR3-1 | | HFR4-1 | |
| TLA015- | TLA0015- | 25 | TLA0015- | 2 | TLA0015- | 31 | TLA0015- | 10 | TLA0015- | 35 | TLA0015- | 15 | TLA0015- | 41 |
| 30 | HFR1-6 | | HCDR1-1 | | HFR2-2 | | HCDR2-5 | | HFR3-2 | | HCDR3-1 | | HFR4-1/ | |
| TLA015- | TLA0015- | 25 | TLA0015- | 2 | TLA0015- | 31 | TLA0015- | 11 | TLA0015- | 35 | TLA0015- | 15 | TLA0015- | 41 |
| 31 | HFR1-6 | | HCDR1-1 | | HFR2-2 | | HCDR2-6 | | HFR3-2 | | HCDR3-1 | | HFR4-1/TLA- | |
| | | | | | | | | | | | | | 0238-HFR4-2 | |
| TLA015- | TLA0015- | 28 | TLA0015- | 2 | TLA0015- | 31 | TLA0015- | 6 | TLA0015- | 35 | TLA0015- | 15 | TLA0015- | 41 |
| 32 | HFR1-9 | | HCDR1-1 | | HFR2-2 | | HCDR2-1 | | HFR3-2 | | HCDR3-1 | | HFR4-1/TLA- | |
| | | | | | | | | | | | | | 0238-HFR4-2 | |
| TLA015- | TLA0015- | 25 | TLA0015- | 2 | TLA0015- | 31 | TLA0015- | 12 | TLA0015- | 35 | TLA0015- | 15 | TLA0015- | 41 |
| 33 | HFR1-6 | | HCDR1-1 | | HFR2-2 | | HCDR2-7 | | HFR3-2 | | HCDR3-1 | | HFR4-1/TLA- | |
| | | | | | | | | | | | | | 0238-HFR4-2 | |
| TLA015- | TLA0015- | 25 | TLA0015- | 2 | TLA0015- | 31 | TLA0015- | 13 | TLA0015- | 35 | TLA0015- | 15 | TLA0015- | 41 |
| 34 | HFR1-6 | | HCDR1-1 | | HFR2-2 | | HCDR2-8 | | HFR3-2 | | HCDR3-1 | | HFR4-1/TLA- | |
| | | | | | | | | | | | | | 0238-HFR4-2 | |
| TLA015- | TLA0015- | 25 | TLA0015- | 3 | TLA0015- | 31 | TLA0015- | 6 | TLA0015- | 35 | TLA0015- | 16 | TLA0015- | 41 |
| 35 | HFR1-6 | | HCDR1-2 | | HFR2-2 | | HCDR2-1 | | HFR3-2 | | HCDR3-2 | | HFR4-1/ | |
| TLA015- | TLA0015- | 25 | TLA0015- | 3 | TLA0015- | 31 | TLA0015- | 6 | TLA0015- | 35 | TLA0015- | 17 | TLA0015- | 41 |
| 36 | HFR1-6 | | HCDR1-2 | | HFR2-2 | | HCDR2-1 | | HFR3-2 | | HCDR3-3 | | HFR4-1 | |
| TLA015- | TLA0015- | 25 | TLA0015- | 3 | TLA0015- | 31 | TLA0015- | 9 | TLA0015- | 35 | TLA0015- | 15 | TLA0015- | 41 |
| 37 | HFR1-6 | | HCDR1-2 | | HFR2-2 | | HCDR2-4 | | HFR3-2 | | HCDR3-1 | | HFR4-1 | |
| TLA015- | TLA0015- | 25 | TLA0015- | 3 | TLA0015- | 31 | TLA0015- | 10 | TLA0015- | 35 | TLA0015- | 15 | TLA0015- | 41 |
| 38 | HFR1-6 | | HCDR1-2 | | HFR2-2 | | HCDR2-5 | | HFR3-2 | | HCDR3-1 | | HFR4-1 | |
| TLA015- | TLA0015- | 25 | TLA0015- | 3 | TLA0015- | 31 | TLA0015- | 11 | TLA0015- | 35 | TLA0015- | 15 | TLA0015- | 41 |
| 39 | HFR1-6 | | HCDR1-2 | | HFR2-2 | | HCDR2-6 | | HFR3-2 | | HCDR3-1 | | HFR4-1/TLA- | |
| | | | | | | | | | | | | | 0238-HFR4-2 | |
| TLA015- | TLA0015- | 28 | TLA0015- | 3 | TLA0015- | 31 | TLA0015- | 6 | TLA0015- | 35 | TLA0015- | 15 | TLA0015- | 41 |
| 40 | HFR1-9 | | HCDR1-2 | | HFR2-2 | | HCDR2-1 | | HFR3-2 | | HCDR3-1 | | HFR4-1 | |
| TLA015- | TLA0015- | 26 | TLA0015- | 2 | TLA0015- | 31 | TLA0015- | 6 | TLA0015- | 35 | TLA0015- | 16 | TLA0015- | 41 |
| 41 | HFR1-7 | | HCDR1-1 | | HFR2-2 | | HCDR2-1 | | HFR3-2 | | HCDR3-2 | | HFR4-1 | |
| TLA015- | TLA0015- | 25 | TLA0015- | 2 | TLA0015- | 31 | TLA0015- | 9 | TLA0015- | 35 | TLA0015- | 16 | TLA0015- | 41 |
| 42 | HFR1-6 | | HCDR1-1 | | HFR2-2 | | HCDR2-4 | | HFR3-2 | | HCDR3-2 | | HFR4-1 | |
| TLA015- | TLA0015- | 25 | TLA0015- | 2 | TLA0015- | 31 | TLA0015- | 10 | TLA0015- | 35 | TLA0015- | 16 | TLA0015- | 41 |
| 43 | HFR1-6 | | HCDR1-1 | | HFR2-2 | | HCDR2-5 | | HFR3-2 | | HCDR3-2 | | HFR4-1 | |
| TLA015- | TLA0015- | 25 | TLA0015- | 2 | TLA0015- | 31 | TLA0015- | 11 | TLA0015- | 35 | TLA0015- | 16 | TLA0015- | 41 |
| 44 | HFR1-6 | | HCDR1-1 | | HFR2-2 | | HCDR2-6 | | HFR3-2 | | HCDR3-2 | | HFR4-1 | |
| TLA015- | TLA0015- | 25 | TLA0015- | 2 | TLA0015- | 31 | TLA0015- | 12 | TLA0015- | 35 | TLA0015- | 16 | TLA0015- | 41 |
| 45 | HFR1-6 | | HCDR1-1 | | HFR2-2 | | HCDR2-7 | | HFR3-2 | | HCDR3-2 | | HFR4-1 | |
| TLA015- | TLA0015- | 26 | TLA0015- | 2 | TLA0015- | 31 | TLA0015- | 6 | TLA0015- | 35 | TLA0015- | 17 | TLA0015- | 41 |
| 46 | HFR1-7 | | HCDR1-1 | | HFR2-2 | | HCDR2-1 | | HFR3-2 | | HCDR3-3 | | HFR4-1 | |
| TLA015- | TLA0015- | 25 | TLA0015- | 2 | TLA0015- | 31 | TLA0015- | 10 | TLA0015- | 35 | TLA0015- | 17 | TLA0015- | 41 |
| 47 | HFR1-6 | | HCDR1-1 | | HFR2-2 | | HCDR2-5 | | HFR3-2 | | HCDR3-3 | | HFR4-1/TLA- | |
| | | | | | | | | | | | | | 0238-HFR4-2 | |

TABLE 1-2

| | VL | | | | | | | | | | | | | |
|---|---|---|---|---|---|---|---|---|---|---|---|---|---|---|
| | | SEQ | | SEQ | | SEQ | | SEQ | | SEQ | | SEQ | | SEQ |
| antibody | LFR1 | ID | CDR1 | ID | LFR2 | ID | LCDR2 | ID | LFR3 | ID | LCDR3 | ID | LFR4 | ID |
| TLA0015 | TL0015- | 46 | TLA0015- | 42 | TL0015- | 49 | TL0015- | 43 | TL0015- | 54 | TL0015- | 44 | TL0015- | 59 |
| (mouse) | LFR1-1 | | LCDR1-1 | | LFR2-1 | | LCDR2-1 | | LFR3-1 | | LCDR3-1 | | LFR4-1 | |
| TLA0015-1 | TL0015- | 47 | TLA0015- | 42 | TL0015- | 50 | TL0015- | 43 | TL0015- | 55 | TL0015- | 44 | TL0015- | 60 |
| | LFR1-2/ | | LCDR1-1 | | LFR2-2 | | LCDR2-1 | | LFR3-2 | | LCDR3-1 | | LFR4-2 | |
| | TLA0241- | | | | | | | | | | | | | |
| | LFR1-1 | | | | | | | | | | | | | |
| TLA0015-2 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-3 | TL0015- | 47 | TLA0015- | 42 | TL0015- | 50 | TL0015- | 43 | TL0015- | 55 | TL0015- | 44 | TL0015- | 60 |
| | LFR1-2/ | | LCDR1-1 | | LFR2-2 | | LCDR2-1 | | LFR3-2 | | LCDR3-1 | | LFR4-2 | |
| | TLA0241- | | | | | | | | | | | | | |
| | LFR1-1 | | | | | | | | | | | | | |
| TLA0015-4 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-5 | TL0015- | 47 | TLA0015- | 42 | TL0015- | 50 | TL0015- | 43 | TL0015- | 55 | TL0015- | 44 | TL0015- | 60 |
| | LFR1-2/ | | LCDR1-1 | | LFR2-2 | | LCDR2-1 | | LFR3-2 | | LCDR3-1 | | LFR4-2 | |
| | TLA0241- | | | | | | | | | | | | | |
| | LFR1-1 | | | | | | | | | | | | | |
| TLA0015-6 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-7 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-8 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-9 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-10 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-11 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-12 | TL0015- | 47 | TLA0015- | 42 | TL0015- | 52 | TL0015- | 43 | TL0015- | 57 | TL0015- | 44 | TL0015- | 61 |
| | LFR1-2/ | | LCDR1-1 | | LFR2-4/ | | LCDR2-1 | | LFR3-4/ | | LCDR3-1 | | LFR4-3/ | |
| | TLA0241- | | | | TLA0241- | | | | TLA0238- | | | | TLA0238- | |
| | LFR1-1 | | | | LFR2-1 | | | | LFR3-4/ | | | | LFR4-3 | |
| | | | | | | | | | TLA0241- | | | | | |
| | | | | | | | | | LFR3-2 | | | | | |
| TLA0015-13 | TL0015- | 47 | TLA0015- | 42 | TL0015- | 52 | TL0015- | 43 | TL0015- | 56 | TL0015- | 44 | TL0015- | 61 |
| | LFR1-2/ | | LCDR1-1 | | LFR2-4/ | | LCDR2-1 | | LFR3-3 | | LCDR3-1 | | LFR4-3/ | |
| | TLA0241- | | | | TLA0241- | | | | | | | | TLA0238- | |
| | LFR1-1 | | | | LFR2-1 | | | | | | | | LFR4-3 | |
| TLA0015-14 | TL0015- | 47 | TLA0015- | 42 | TL0015- | 52 | TL0015- | 43 | TL0015- | 57 | TL0015- | 44 | TL0015- | 61 |
| | LFR1-2/ | | LCDR1-1 | | LFR2-4/ | | LCDR2-1 | | LFR3-4/ | | LCDR3-1 | | LFR4-3/ | |
| | TLA0241- | | | | TLA0241- | | | | TLA0238- | | | | TLA0238- | |
| | LFR1-1 | | | | LFR2-1 | | | | LFR3-4/ | | | | LFR4-3 | |
| | | | | | | | | | TLA0241- | | | | | |
| | | | | | | | | | LFR3-2 | | | | | |
| TLA0015-15 | TL0015- | 47 | TLA0015- | 42 | TL0015- | 52 | TL0015- | 43 | TL0015- | 57 | TL0015- | 44 | TL0015- | 61 |
| | LFR1-2/ | | LCDR1-1 | | LFR2-4/ | | LCDR2-1 | | LFR3-4/ | | LCDR3-1 | | LFR4-3/ | |
| | TLA0241- | | | | TLA0241- | | | | TLA0238- | | | | TLA0238- | |
| | LFR1-1 | | | | LFR2-1 | | | | LFR3-4/ | | | | LFR4-3 | |
| | | | | | | | | | TLA0241- | | | | | |
| | | | | | | | | | LFR3-2 | | | | | |
| TLA0015-16 | TL0015- | 47 | TLA0015- | 42 | TL0015- | 52 | TL0015- | 43 | TL0015- | 56 | TL0015- | 44 | TL0015- | 61 |
| | LFR1-2/ | | LCDR1-1 | | LFR2-4/ | | LCDR2-1 | | LFR3-3 | | LCDR3-1 | | LFR4-3/ | |
| | TLA0241- | | | | TLA0241- | | | | | | | | TLA0238- | |
| | LFR1-1 | | | | LFR2-1 | | | | | | | | LFR4-3 | |
| TLa0015-17 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLa0015-18 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLa0015-19 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLa0015-20 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLa0015-21 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-22 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-23 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-24 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-25 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-26 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-27 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-28 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-29 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-30 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-31 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-32 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-33 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-34 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-35 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-36 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-37 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-38 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-39 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-40 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-41 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-42 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-43 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-44 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-45 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-46 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-47 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |

### Isolated Antigen-Binding Protein-TLA-0238

In one aspect, the present disclosure provides an antigen-binding protein which specifically binds to TL1A and comprises a heavy chain variable region (VH). The VH comprises at least one heavy chain complementarity determining regions (HCDR). The VH may include at least one, two or three of HCDR1, HCDR2 or HCDR3. In some embodiments, HCDR 1, HCDR3 and HCDR3 are sequences as set forth in SEQ ID NO: 107, 115 and 120 respectively.

HCDR3 of the antigen-binding protein may include amino acid sequence as set forth in SEQ ID NO: 120. For example, the sequence of HCDR3 of the antigen-binding protein may be defined by Kabat.

HCDR2 of the antigen-binding protein may include amino acid sequence as set forth in SEQ ID NO: 115. For example, the sequence of HCDR2 of the antigen-binding protein may be defined by Kabat.

In some embodiments, HCDR2 may be QIRLKSDNYATHYAX28X29VKG (SEQ ID NO:115). X28 may be A or D or E. X29 may be Por S.

In some embodiments, HCDR2 may include an amino acid sequence as set forth in any one of SEQ ID NOs: 116-119.

HCDR1 of the antigen-binding protein may include amino acid sequence as set forth in SEQ ID NO: 107. For example, the sequence of HCDR1 of the antigen-binding protein may be defined by Kabat.

In some embodiments, HCDR1 may be NYX30MX31 (SEQ ID NO:107). X30 may be I or L or M or Y or W. X31 may be N or H or S.

In some embodiments, HCDR1 may include an amino acid as set forth in any one of SEQ ID NOs: 108-114.

In some embodiments, the VH comprises HFR1, and C-terminus of HFR1 is directly or indirectly linked to the N-terminus of HCDR1. For example, the sequence of HFR1 of the antigen-binding protein may be defined by Kabat. HFR1 comprises an amino acid as set forth in SEQ ID NO: 121.

In some embodiments, HFR1 is

### X32VX33X34X35ESGGGX36VX37PGX38X39LX40LSCX41ASGFTFS (SEQ ID NO:121). X32 may be E or Q. X33 may be K or Q.X34 may be Lor V.X35 may be E or V.X36 may be Lor V.X37 may be K or Q. X38 may be G or R.X39 may be A or S.X40 may be K or R.X41 may be A or I.

In some embodiments, HFR1 is selected from an amino acid sequence as set forth in any one of SEQ ID NOs: 122-132.

In some embodiments, VH comprises HFR2, and HFR2 is located between HCDR1 and HCDR2. For example, the sequence of HFR2 of the antigen-binding protein may be defined by Kabat. HFR2 comprises an amino acid sequence as set forth in SEQ ID NO:133.

In some embodiments, HFR2 is WVRQX42PX43KGLEWVX44 (SEQ ID NO:133). X42 may be A or S.X43 may be E or G.X44 may be A or G.

In some embodiments, HFR2 is selected from an amino acid sequence as set forth in any one of SEQ ID NOs: 134-135 and 30.

In some embodiments, VH comprises HFR3, and HFR3 is located between HCDR2 and HCDR3. For example, the sequence of HFR3 of the antigen-binding protein may be defined by Kabat. HFR3 comprises an amino acid as set forth in SEQ ID NO:136.

In some embodiments, HFR3 is

X45FX46ISRDX47X48KX49X50X51YLQMNSLX52X53EDX54AVYYCTP (SEQ ID NO:136). X45 may be N or R.X46 may be A or T.X47 may be D or N.X48 may be A or S.X49 may be Nor S.X50 may be S or T.X51 may be A or Lor V.X52 may be Kor R.X53 may be A or D or T.X54 may be M or T.

In some embodiments, HFR3 is selected from an amino acid sequence as set forth in any one of SEQ ID NOs: 137-148.

In some embodiments, VH comprises HFR4, and N-terminus of HFR4 is directly or indirectly linked to C-terminus of HCDR3. For example, the sequence of HFR4 of the antigen-binding protein may be defined by Kabat. HFR4 comprises an amino acid sequence as set forth in SEQ ID NO:149.

In some embodiments, HFR4 is WGQGT X55X56TVSS (SEQ ID NO:149). X55 may be L or M OR H or T.X56 may be Lor V.

In some embodiments, the isolated antigen-binding protein further comprises a light chain variable region (VL), an VL comprises at least one light chain complementarity determining regions (LCDR). VL comprises LCDR1, LCDR2 and LCDR3, which is selected from an amino acid sequence as set forth in any one of SEQ ID NO: 154, SEQ ID NO: 159 and SEQ ID NO:163 accordingly.

In some embodiments, VL comprises LCDR3 including an amino acid sequence as set forth in SEQ ID NO: 163. For example, the sequence of LCDR3 of the antigen-binding protein may be defined by Kabat.

In some embodiments, LCDR3 is FQEX57X58HPFT (SEQ ID NO:163). X57 may be N or S or E.X58 may be G or E or A.

In some embodiments, LCDR3 is selected from an amino acid sequence as set forth in any one of SEQ ID NOs: 164-168.

In some embodiments, VL comprises LCDR2 including an amino acid sequence as set forth in SEQ ID NO: 159. For example, the sequence of LCDR2 of the antigen-binding protein may be defined by Kabat.

In some embodiments, LCDR2 is DTSNX59AX60 (SEQ ID NO:159). X59 may be L or R.X60 may be S or T.

In some embodiments, LCDR2 is selected from an amino acid sequence as set forth in any one of SEQ ID NOs: 160-162.

In some embodiments, VL comprises LCDR1 including an amino acid sequence as set forth in SEQ ID NO:42. For example, the sequence of LCDR1 of the antigen-binding protein may be defined by Kabat.

In some embodiments, LCDR1 is SASSSVSYX61X62 (SEQ ID NO:154). X61 may be Lor M.X62 may be A or H or T.

In some embodiments, LCDR1 is selected from an amino acid sequence as set forth in any one of SEQ ID NOs: 155-158.

In some embodiments, VL comprises the LFR1, and C-terminus of the LFR1 is directly or indirectly linked to N-terminus of LCDR1. For example, the sequence of the LFR1 of the antigen-binding protein may be defined by Kabat. the LFR1 comprises an amino acid sequence as set forth in SEQ ID NO:169.

In some embodiments, the LFR1 is

X63X64X65X66TQSPX67X68X69SX70X71X72GX73X74X75TX76X77C (SEQ ID NO:169). X63 may be E or D.X64 may be I or N or Tor V.X65 may be Q or V.X66 may be Lor M.X67 may be A or S. X68 may be For I or S or T.X69 may be Lor M or V. X70 may be A or Lor V. X71 may be S or T.X72 may be Por V.X73 may be D or E. X74 may be Kor R.X75 may be A or V.X76 may be I or M.X77 may be S or T.

In some embodiments, the LFR1 comprises an amino acid sequence as set forth in any one of SEQ ID NOs: 170-179.

In some embodiments, VL comprises the LFR2, and the LFR2 is located between LCDR1 and LCDR2. For example, the sequence of the LFR2 of the antigen-binding protein may be defined by Kabat, the LFR2 comprises an amino acid sequence as set forth in SEQ ID NO:180.

In some embodiments, the LFR2 is WYQQKP X78 X79 X80P X81LWIY (SEQ ID NO: 180).X78 may be D or G.X79 may be K or Q.X80 may be A or S.X81 may be K or Q or R.

In some embodiments, the LFR2 comprises an amino acid sequence as set forth in any one of SEQ ID NOs: 181-188.

In some embodiments, VL comprises the LFR3, the LFR3 is located between LCDR2 and LCDR3. For example, the sequence of the LFR3 of the antigen-binding protein may be defined by Kabat. The LFR3 comprises an amino acid sequence as set forth in SEQ ID NO:189.

In some embodiments, the LFR3 is GVP

X82RFSGSGSGX83X84X85TLTISSX86X87X88EDX89AX90YYC (SEQ ID NO:189). X82 may be A or D or G or S. X83 may be N or T.X84 may be D or S. X85 may be For Y. X86 may be Lor M.X87 may be E or Q. X88 may be A or P.X89 may be A or For V. X90 may be Tor V.

In some embodiments, the LFR3 comprises an amino acid sequence as set forth in any one of SEQ ID NOs: 190-197 and 57.

In some embodiments, the isolated antigen-binding protein comprises the LFR4, and N-terminus of the LFR4 is directly or indirectly linked to C terminus of LCDR3. For example, the sequence of the LFR4 of the antigen-binding protein may be defined by Kabat. The LFR4 comprises an amino acid sequence as set forth in SEQ ID NO:198.

In some embodiments, the LFR4 is FG X91GTK X92EX93K (SEQ ID NO: 198). X91 may be G or Q. X92 may be Lor V. X93 may be I or M.

In some embodiments, the LFR4 comprises an amino acid sequence as set forth in any one of SEQ ID NOs: 199-201 and 61.

In some embodiments, VH comprises sequences as set forth in SEQ ID NOs: 202-220, 332-334.

In some embodiments, VL comprises sequences as set forth in SEQ ID NOs: 221-234, 335-338. In some embodiments, the antigen-binding proteins described above are mentioned in Example 4, Tables 5, 6 and 7.

In some embodiments, the distribution of antibodies for the TLA-0015 line is shown in the following table, with Table 1-3 showing the distribution of heavy chain components and Table 1-4 showing the distribution of light chain components. The technical solution of the present application includes, for the same antibody, a combination of HCDR1-3 in Table 1-3 and LCDR1-3 in Table 1-4. The technical solution of the present application includes, for the same antibody, a combination of VH in Table 1-3 and VL in Table 1-4.

TABLE 1-3

| | VH | | | | | | |
|---|---|---|---|---|---|---|---|
| | | SEQ | | SEQ | | SEQ | |
| antibody | HFR1 | ID | HCDR1 | ID | HFR2 | ID | HCDR2 |
| TLA0238 | | | TLA-0238- | 108 | | 134 | TLA-0238- |
| (mouse) | | | HCDR1-1 | | | | HCDR2-1 |
| TLA0238-1 | TLA-0238- | 123 | TLA-0238- | 108 | TLA-0238- | 135 | TLA-0238- |
| | HFR1-2 | | HCDR1-1 | | HFR2-2 | | HCDR2-1 |
| TLA0238-2 | TLA-0238- | 123 | TLA-0238- | 108 | TLA-0238- | 135 | TLA-0238- |
| | HFR1-2 | | HCDR1-1 | | HFR2-2 | | HCDR2-1 |
| TLA0238-3 | TLA-0238- | 124 | TLA-0238- | 108 | TLA-0238- | 135 | TLA-0238- |
| | HFR1-3 | | HCDR1-1 | | HFR2-2 | | HCDR2-1 |
| TLA0238-4 | TLA-0238- | 124 | TLA-0238- | 108 | TLA-0238- | 135 | TLA-0238- |
| | HFR1-3 | | HCDR1-1 | | HFR2-2 | | HCDR2-1 |
| TLA0238-2-1 | TLA-0238- | 123 | TLA-0238- | 108 | TLA-0238- | 135 | TLA-0238- |
| | HFR1-2 | | HCDR1-1 | | HFR2-2 | | HCDR2-1 |
| TLA0238-2-2 | TLA-0238- | 123 | TLA-0238- | 108 | TLA-0238- | 135 | TLA-0238- |
| | HFR1-2 | | HCDR1-1 | | HFR2-2 | | HCDR2-1 |
| TLA0238-5 | TLA-0238- | 125 | TLA-0238- | 108 | TLA-0238- | 135 | TLA-0238- |
| | HFR1-4 | | HCDR1-1 | | HFR2-2 | | HCDR2-2 |
| TLA0238-6 | TLA-0238- | 126 | TLA-0238- | 108 | TLA-0238- | 135 | TLA-0238- |
| | HFR1-5 | | HCDR1-1 | | HFR2-2 | | HCDR2-2 |
| TLA0238-7 | TLA-0238- | 127 | TLA-0238- | 109 | TLA0015- | 30 | TLA-0238- |
| | HFR1-6 | | HCDR1-2 | | HFR2-1/TLA- | | HCDR2-3 |
| | | | | | 0238-HFR2-3 | | |
| TLA0238-8 | TLA-0238- | 128 | TLA-0238- | 109 | TLA0015- | 30 | TLA-0238- |
| | HFR1-7 | | HCDR1-2 | | HFR2-1/TLA- | | HCDR2-3 |
| | | | | | 0238-HFR2-3 | | |
| TLA0238-9 | TLA-0238- | 129 | TLA-0238- | 109 | TLA0015- | 30 | TLA-0238- |
| | HFR1-8 | | HCDR1-2 | | HFR2-1/TLA- | | HCDR2-3 |
| | | | | | 0238-HFR2-3 | | |
| TLA0238-10 | TLA-0238- | 127 | TLA-0238- | 109 | TLA0015- | 30 | TLA-0238- |
| | HFR1-6 | | HCDR1-2 | | HFR2-1/TLA- | | HCDR2-3 |
| | | | | | 0238-HFR2-3 | | |
| TLA0238-11 | TLA-0238- | 125 | TLA-0238- | 108 | TLA-0238- | 135 | TLA-0238- |
| | HFR1-4 | | HCDR1-1 | | HFR2-2 | | HCDR2-2 |
| TLA0238-12 | TLA-0238- | 130 | TLA-0238- | 109 | TLA0015- | 30 | TLA-0238- |
| | HFR1-9 | | HCDR1-2 | | HFR2-1/TLA- | | HCDR2-3 |
| | | | | | 0238-HFR2-3 | | |
| TLA0238-13 | TLA-0238- | 131 | TLA-0238- | 108 | TLA-0238- | 135 | TLA-0238- |
| | HFR1-10 | | HCDR1-1 | | HFR2-2 | | HCDR2-4 |
| TLA0238-14 | TLA-0238- | 132 | TLA-0238- | 110 | TLA0015- | 30 | TLA-0238- |
| | HFR1-11 | | HCDR1-3 | | HFR2-1/TLA- | | HCDR2-3 |
| | | | | | 0238-HFR2-3 | | |
| TLA0238-15 | TLA-0238- | 125 | TLA-0238- | 111 | TLA-0238- | 135 | TLA-0238- |
| | HFR1-4 | | HCDR1-4 | | HFR2-2 | | HCDR2-2 |
| TLA0238-16 | TLA-0238- | 125 | TLA-0238- | 111 | TLA-0238- | 135 | TLA-0238- |
| | HFR1-4 | | HCDR1-4 | | HFR2-2 | | HCDR2-2 |
| TLA0238-17 | TLA-0238- | 125 | TLA-0238- | 112 | TLA-0238- | 135 | TLA-0238- |
| | HFR1-4 | | HCDR1-5 | | HFR2-2 | | HCDR2-2 |
| TLA0238-18 | TLA-0238- | 125 | TLA-0238- | 112 | TLA-0238- | 135 | TLA-0238- |
| | HFR1-4 | | HCDR1-5 | | HFR2-2 | | HCDR2-2 |
| TLA0238-19 | TLA-0238- | 125 | TLA-0238- | 113 | TLA-0238- | 135 | TLA-0238- |
| | HFR1-4 | | HCDR1-6 | | HFR2-2 | | HCDR2-2 |
| TLA0238-20 | TLA-0238- | 125 | TLA-0238- | 113 | TLA-0238- | 135 | TLA-0238- |
| | HFR1-4 | | HCDR1-6 | | HFR2-2 | | HCDR2-2 |
| TLA0238-21 | TLA-0238- | 125 | TLA-0238- | 111 | TLA-0238- | 135 | TLA-0238- |
| | HFR1-4 | | HCDR1-4 | | HFR2-2 | | HCDR2-2 |
| TLA0238-22 | TLA-0238- | 125 | TLA-0238- | 111 | TLA-0238- | 135 | TLA-0238- |
| | HFR1-4 | | HCDR1-4 | | HFR2-2 | | HCDR2-2 |
| TLA0238-23 | TLA-0238- | 125 | TLA-0238- | 112 | TLA-0238- | 135 | TLA-0238- |
| | HFR1-4 | | HCDR1-5 | | HFR2-2 | | HCDR2-2 |
| TLA0238-24 | TLA-0238- | 125 | TLA-0238- | 112 | TLA-0238- | 135 | TLA-0238- |
| | HFR1-4 | | HCDR1-5 | | HFR2-2 | | HCDR2-2 |
| TLA0238-25 | TLA-0238- | 125 | TLA-0238- | 113 | TLA-0238- | 135 | TLA-0238- |
| | HFR1-4 | | HCDR1-6 | | HFR2-2 | | HCDR2-2 |
| TLA0238-26 | TLA-0238- | 125 | TLA-0238- | 113 | TLA-0238- | 135 | TLA-0238- |
| | HFR1-4 | | HCDR1-6 | | HFR2-2 | | HCDR2-2 |
| TLA0238-27 | TLA-0238- | 125 | TLA-0238- | 108 | TLA-0238- | 135 | TLA-0238- |
| | HFR1-4 | | HCDR1-1 | | HFR2-2 | | HCDR2-2 |
| TLA0238-28 | TLA-0238- | 125 | TLA-0238- | 108 | TLA-0238- | 135 | TLA-0238- |
| | HFR1-4 | | HCDR1-1 | | HFR2-2 | | HCDR2-2 |
| TLA0238-29 | TLA-0238- | 125 | TLA-0238- | 114 | TLA-0238- | 135 | TLA-0238- |
| | HFR1-4 | | HCDR1-7 | | HFR2-2 | | HCDR2-2 |
| TLA0238-30 | TLA-0238- | 125 | TLA-0238- | 114 | TLA-0238- | 135 | TLA-0238- |
| | HFR1-4 | | HCDR1-7 | | HFR2-2 | | HCDR2-2 |

| | VH | | | | | | |
|---|---|---|---|---|---|---|---|
| | SEQ | | SEQ | | SEQ | | SEQ |
| antibody | ID | HFR3 | ID | HCDR3 | ID | HFR4 | ID |
| TLA0238 | 116 | | | TLA-0238- | 120 | | |
| (mouse) | | | | HCDR3-1 | | | |
| TLA0238-1 | 116 | TLA-0238- | 138 | TLA-0238- | 120 | TLA0015- | 41 |
| | | HFR3-2 | | HCDR3-1 | | HFR4-1/TLA- | |
| | | | | | | 0238-HFR4-2 | |
| TLA0238-2 | 116 | TLA-0238- | 138 | TLA-0238- | 120 | TLA0015- | 41 |
| | | HFR3-2 | | HCDR3-1 | | HFR4-1/TLA- | |
| | | | | | | 0238-HFR4-2 | |
| TLA0238-3 | 116 | TLA-0238- | 139 | TLA-0238- | 120 | TLA-0238- | 151 |
| | | HFR3-3 | | HCDR3-1 | | HFR4-3 | |
| TLA0238-4 | 116 | TLA-0238- | 139 | TLA-0238- | 120 | TLA-0238- | 151 |
| | | HFR3-3 | | HCDR3-1 | | HFR4-3 | |
| TLA0238-2-1 | 116 | TLA-0238- | 140 | TLA-0238- | 120 | TLA0015- | 41 |
| | | HFR3-4 | | HCDR3-1 | | HFR4-1/TLA- | |
| | | | | | | 0238-HFR4-2 | |
| TLA0238-2-2 | 116 | TLA-0238- | 141 | TLA-0238- | 120 | TLA0015- | 41 |
| | | HFR3-5 | | HCDR3-1 | | HFR4-1/TLA- | |
| | | | | | | 0238-HFR4-2 | |
| TLA0238-5 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-6 | 117 | TLA-0238- | 142 | TLA-0238- | 120 | TLA-0238- | 152 |
| | | HFR3-6 | | HCDR3-1 | | HFR4-4 | |
| TLA0238-7 | 118 | TLA-0238- | 143 | TLA-0238- | 120 | TLA0015- | 41 |
| | | HFR3-7 | | HCDR3-1 | | HFR4-1/TLA- | |
| | | | | | | 0238-HFR4-2 | |
| TLA0238-8 | 118 | TLA-0238- | 144 | TLA-0238- | 120 | TLA0015- | 41 |
| | | HFR3-8 | | HCDR3-1 | | HFR4-1/TLA- | |
| | | | | | | 0238-HFR4-2 | |
| TLA0238-9 | 118 | TLA-0238- | 145 | TLA-0238- | 120 | TLA0015- | 41 |
| | | HFR3-9 | | HCDR3-1 | | HFR4-1/TLA- | |
| | | | | | | 0238-HFR4-2 | |
| TLA0238-10 | 118 | TLA-0238- | 146 | TLA-0238- | 120 | TLA0015- | 41 |
| | | HFR3-10 | | HCDR3-1 | | HFR4-1/TLA- | |
| | | | | | | 0238-HFR4-2 | |
| TLA0238-11 | 117 | TLA-0238- | 142 | TLA-0238- | 120 | TLA-0238- | 152 |
| | | HFR3-6 | | HCDR3-1 | | HFR4-4 | |
| TLA0238-12 | 118 | TLA-0238- | 147 | TLA-0238- | 120 | TLA0015- | 41 |
| | | HFR3-11 | | HCDR3-1 | | HFR4-1/TLA- | |
| | | | | | | 0238-HFR4-2 | |
| TLA0238-13 | 119 | TLA-0238- | 148 | TLA-0238- | 120 | TLA-0238- | 153 |
| | | HFR3-12 | | HCDR3-1 | | HFR4-5 | |
| TLA0238-14 | 118 | TLA-0238- | 145 | TLA-0238- | 120 | TLA0015- | 41 |
| | | HFR3-9 | | HCDR3-1 | | HFR4-1/TLA- | |
| | | | | | | 0238-HFR4-2 | |
| TLA0238-15 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-16 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-17 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-18 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-19 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-20 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-21 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-22 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-23 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-24 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-25 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-26 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-27 | 111 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-28 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-29 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |
| TLA0238-30 | 117 | TLA-0238- | 142 | TLA-0238- | 120 | TLA0015- | 41 |
| | | HFR3-6 | | HCDR3-1 | | HFR4-1/TLA- | |
| | | | | | | 0238-HFR4-2 | |

TABLE 1-4

| | VL | | | | | | | | | | | | | |
|---|---|---|---|---|---|---|---|---|---|---|---|---|---|---|
| | | SEQ | | SEQ | | SEQ | | SEQ | | SEQ | | SEQ | | SEQ |
| antibody | LFR1 | ID | CDR1 | ID | LFR2 | ID | LCDR2 | ID | LFR3 | ID | LCDR3 | ID | LFR4 | ID |
| TLA0238 | | | TLA0238- | 155 | | | TLA0238- | 160 | | | TLA0238- | 164 | | |
| (mouse) | | | LCDR1-1 | | | | LCDR2-1 | | | | LCDR3-1 | | | |
| TLA0238-1 | TLA0238- | 171 | TLA0238- | 155 | TLA0238- | 182 | TLA0238- | 160 | TLA0238- | 191 | TLA0238- | 164 | TLA0238- | 200 |
| | LFR1-2 | | LCDR1-1 | | LFR2-2 | | LCDR2-1 | | LFR3-2 | | LCDR3-1 | | LFR4-2 | |
| TLA0238-2 | TLA0238- | 172 | TLA0238- | 155 | TLA0238- | 183 | TLA0238- | 160 | TLA0238- | 192 | TLA0238- | 164 | TL0015- | 61 |
| | LFR1-3 | | LCDR1-1 | | LFR2-3 | | LCDR2-1 | | LFR3-3 | | LCDR3-1 | | LFR4-3/ | |
| | | | | | | | | | | | | | TLA0238- | |
| | | | | | | | | | | | | | LFR4-3 | |
| TLA0238-3 | TLA0238- | 171 | TLA0238- | 155 | TLA0238- | 182 | TLA0238- | 160 | TLA0238- | 191 | TLA0238- | 164 | TLA0238- | 200 |
| | LFR1-2 | | LCDR1-1 | | LFR2-2 | | LCDR2-1 | | LFR3-2 | | LCDR3-1 | | LFR4-2 | |
| TLA0238-4 | TLA0238- | 172 | TLA0238- | 155 | TLA0238- | 183 | TLA0238- | 160 | TLA0238- | 192 | TLA0238- | 164 | TL0015- | 61 |
| | LFR1-3 | | LCDR1-1 | | LFR2-3 | | LCDR2-1 | | LFR3-3 | | LCDR3-1 | | LFR4-3/ | |
| | | | | | | | | | | | | | TLA0238- | |
| | | | | | | | | | | | | | LFR4-3 | |
| TLA0238-2-1 | TLA0238- | 172 | TLA0238- | 155 | TLA0238- | 183 | TLA0238- | 160 | TLA0238- | 192 | TLA0238- | 164 | TL0015- | 61 |
| | LFR1-3 | | LCDR1-1 | | LFR2-3 | | LCDR2-1 | | LFR3-3 | | LCDR3-1 | | LFR4-3/ | |
| | | | | | | | | | | | | | TLA0238- | |
| | | | | | | | | | | | | | LFR4-3 | |
| TLA0238-2-2 | TLA0238- | 172 | TLA0238- | 155 | TLA0238- | 183 | TLA0238- | 160 | TLA0238- | 192 | TLA0238- | 164 | TL0015- | 61 |
| | LFR1-3 | | LCDR1-1 | | LFR2-3 | | LCDR2-1 | | LFR3-3 | | LCDR3-1 | | LFR4-3/ | |
| | | | | | | | | | | | | | TLA0238- | |
| | | | | | | | | | | | | | LFR4-3 | |
| TLA0238-5 | TLA0238- | 173 | TLA0238- | 155 | TLA0238- | 183 | TLA0238- | 160 | TL0015- | 57 | TLA0238- | 164 | TLA0238- | 201 |
| | LFR1-4/ | | LCDR1-1 | | LFR2-3 | | LCDR2-1 | | LFR3-4/ | | LCDR3-1 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-6 | TLA0238- | 173 | TLA0238- | 155 | TLA0238- | 183 | TLA0238- | 160 | TL0015- | 57 | TLA0238- | 164 | TLA0238- | 201 |
| | LFR1-4/ | | LCDR1-1 | | LFR2-3 | | LCDR2-1 | | LFR3-4/ | | LCDR3-1 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-7 | TLA0238- | 174 | TLA0238- | 156 | TLA0238- | 184 | TLA0238- | 161 | TLA0238- | 193 | TLA0238- | 164 | TLA0238- | 199 |
| | LFR1-5 | | LCDR1-2 | | LFR2-4 | | LCDR2-2 | | LFR3-5 | | LCDR3-1 | | LFR4-1 | |
| TLA0238-8 | TLA0238- | 175 | TLA0238- | 157 | TLA0238- | 185 | TLA0238- | 161 | TLA0238- | 194 | TLA0238- | 164 | TLA0238- | 199 |
| | LFR1-6 | | LCDR1-3 | | LFR2-5 | | LCDR2-2 | | LFR3-6 | | LCDR3-1 | | LFR4-1 | |
| TLA0238-9 | TLA0238- | 176 | TLA0238- | 157 | TLA0238- | 186 | TLA0238- | 162 | TLA0238- | 195 | TLA0238- | 164 | TLA0238- | 199 |
| | LFR1-7 | | LCDR1-3 | | LFR2-6 | | LCDR2-3 | | LFR3-7 | | LCDR3-1 | | LFR4-1 | |
| TLA0238-10 | TLA0238- | 177 | TLA0238- | 156 | TLA0238- | 187 | TLA0238- | 161 | TLA0238- | 196 | TLA0238- | 164 | TLA0238- | 199 |
| | LFR1-8 | | LCDR1-2 | | LFR2-7 | | LCDR2-2 | | LFR3-8 | | LCDR3-1 | | LFR4-1 | |
| TLA0238-11 | TLA0238- | 173 | TLA0238- | 155 | TLA0238- | 183 | TLA0238- | 160 | TL0015- | 57 | TLA0238- | 164 | TLA0238- | 201 |
| | LFR1-4/ | | LCDR1-1 | | LFR2-3 | | LCDR2-1 | | LFR3-4/ | | LCDR3-1 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-12 | TLA0238- | 178 | TLA0238- | 158 | TLA0238- | 188 | TLA0238- | 160 | TLA0238- | 196 | TLA0238- | 164 | TLA0238- | 199 |
| | LFR1-9 | | LCDR1-4 | | LFR2-8 | | LCDR2-1 | | LFR3-8 | | LCDR3-1 | | LFR4-1 | |
| TLA0238-13 | TLA0238- | 173 | TLA0238- | 155 | TLA0238- | 183 | TLA0238- | 160 | TL0015- | 57 | TLA0238- | 164 | TLA0238- | 201 |
| | LFR1-4/ | | LCDR1-1 | | LFR2-3 | | LCDR2-1 | | LFR3-4/ | | LCDR3-1 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-14 | TLA0238- | 179 | TLA0238- | 156 | TLA0238- | 187 | TLA0238- | 162 | TLA0238- | 197 | TLA0238- | 164 | TLA0238- | 199 |
| | LFR1-10 | | LCDR1-2 | | LFR2-7 | | LCDR2-3 | | LFR3-9 | | LCDR3-1 | | LFR4-1 | |
| TLA0238-15 | TLA0238- | 173 | TLA0238- | 155 | TLA0238- | 183 | TLA0238- | 160 | TL0015- | 57 | TLA0238- | 165 | TLA0238- | 201 |
| | LFR1-4/ | | LCDR1-1 | | LFR2-3 | | LCDR2-1 | | LFR3-4/ | | LCDR3-2 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-16 | TLA0238- | 173 | TLA0238- | 155 | TLA0238- | 183 | TLA0238- | 160 | TL0015- | 57 | TLA0238- | 166 | TLA0238- | 201 |
| | LFR1-4/ | | LCDR1-1 | | LFR2-3 | | LCDR2-1 | | LFR3-4/ | | LCDR3-3 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-17 | TLA0238- | 173 | TLA0238- | 155 | TLA0238- | 183 | TLA0238- | 160 | TL0015- | 57 | TLA0238- | 165 | TLA0238- | 201 |
| | LFR1-4/ | | LCDR1-1 | | LFR2-3 | | LCDR2-1 | | LFR3-4/ | | LCDR3-2 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-18 | TLA0238- | 173 | TLA0238- | 155 | TLA0238- | 183 | TLA0238- | 160 | TL0015- | 57 | TLA0238- | 166 | TLA0238- | 201 |
| | LFR1-4/ | | LCDR1-1 | | LFR2-3 | | LCDR2-1 | | LFR3-4/ | | LCDR3-3 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-19 | TLA0238- | 173 | TLA0238- | 155 | TLA0238- | 183 | TLA0238- | 160 | TL0015- | 57 | TLA0238- | 165 | TLA0238- | 201 |
| | LFR1-4/ | | LCDR1-1 | | LFR2-3 | | LCDR2-1 | | LFR3-4/ | | LCDR3-2 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-20 | TLA0238- | 173 | TLA0238- | 155 | TLA0238- | 183 | TLA0238- | 160 | TL0015- | 57 | TLA0238- | 166 | TLA0238- | 201 |
| | LFR1-4/ | | LCDR1-1 | | LFR2-3 | | LCDR2-1 | | LFR3-4/ | | LCDR3-3 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-21 | TLA0238- | 173 | TLA0238- | 155 | TLA0238- | 183 | TLA0238- | 160 | TL0015- | 57 | TLA0238- | 167 | TLA0238- | 201 |
| | LFR1-4/ | | LCDR1-1 | | LFR2-3 | | LCDR2-1 | | LFR3-4/ | | LCDR3-4 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-22 | TLA0238- | 173 | TLA0238- | 155 | TLA0238- | 183 | TLA0238- | 160 | TL0015- | 57 | TLA0238- | 168 | TLA0238- | 201 |
| | LFR1-4/ | | LCDR1-1 | | LFR2-3 | | LCDR2-1 | | LFR3-4/ | | LCDR3-5 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-23 | TLA0238- | 173 | TLA0238- | 155 | TLA0238- | 183 | TLA0238- | 160 | TL0015- | 57 | TLA0238- | 167 | TLA0238- | 201 |
| | LFR1-4/ | | LCDR1-1 | | LFR2-3 | | LCDR2-1 | | LFR3-4/ | | LCDR3-4 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-24 | TLA0238- | 173 | TLA0238- | 155 | TLA0238- | 183 | TLA0238- | 160 | TL0015- | 57 | TLA0238- | 168 | TLA0238- | 201 |
| | LFR1-4/ | | LCDR1-1 | | LFR2-3 | | LCDR2-1 | | LFR3-4/ | | LCDR3-5 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-25 | TLA0238- | 173 | TLA0238- | 155 | TLA0238- | 183 | TLA0238- | 160 | TL0015- | 57 | TLA0238- | 167 | TLA0238- | 201 |
| | LFR1-4/ | | LCDR1-1 | | LFR2-3 | | LCDR2-1 | | LFR3-4/ | | LCDR3-4 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-26 | TLA0238- | 173 | TLA0238- | 155 | TLA0238- | 183 | TLA0238- | 160 | TL0015- | 57 | TLA0238- | 168 | TLA0238- | 201 |
| | LFR1-4/ | | LCDR1-1 | | LFR2-3 | | LCDR2-1 | | LFR3-4/ | | LCDR3-5 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-27 | TLA0238- | 173 | TLA0238- | 155 | TLA0238- | 183 | TLA0238- | 160 | TL0015- | 57 | TLA0238- | 167 | TLA0238- | 201 |
| | LFR1-4/ | | LCDR1-1 | | LFR2-3 | | LCDR2-1 | | LFR3-4/ | | LCDR3-4 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-28 | TLA0238- | 173 | TLA0238- | 155 | TLA0238- | 183 | TLA0238- | 160 | TL0015- | 57 | TLA0238- | 168 | TLA0238- | 201 |
| | LFR1-4/ | | LCDR1-1 | | LFR2-3 | | LCDR2-1 | | LFR3-4/ | | LCDR3-5 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-29 | TLA0238- | 173 | TLA0238- | 155 | TLA0238- | 183 | TLA0238- | 160 | TL0015- | 57 | TLA0238- | 167 | TLA0238- | 201 |
| | LFR1-4/ | | LCDR1-1 | | LFR2-3 | | LCDR2-1 | | LFR3-4/ | | LCDR3-4 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-30 | TLA0238- | 173 | TLA0238- | 155 | TLA0238- | 183 | TLA0238- | 160 | TL0015- | 57 | TLA0238- | 168 | TLA0238- | 201 |
| | LFR1-4/ | | LCDR1-1 | | LFR2-3 | | LCDR2-1 | | LFR3-4/ | | LCDR3-5 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |

### Isolated Antigen-Binding Protein-TLA-0241

In one aspect, the present disclosure provides an antigen-binding protein which specifically binds to TL1A and comprises a heavy chain variable region (VH). The VH comprises at least one heavy chain complementarity determining regions (HCDR). The VH may include at least one, two or three of HCDR1, HCDR2 or HCDR3. In some embodiments, HCDR 1, HCDR3 and HCDR3 are sequences as set forth in SEQ ID NO:235, 238 and 242 respectively.

HCDR3 of the antigen-binding protein may include amino acid sequence as set forth in SEQ ID NO: 242. For example, the sequence of HCDR3 of the antigen-binding protein may be defined by Kabat.

HCDR2 of the antigen-binding protein may include amino acid sequence as set forth in SEQ ID NO: 238. For example, the sequence of HCDR2 of the antigen-binding protein may be defined by Kabat.

In some embodiments, HCDR2 may be DINPNNGRTTYX94X95X96X97X98G (SEQ ID NO: 238). X94 may be A or N. X95 may be D or Q. X96 may be K or S. X97 may be For V. X98 may be K or Q or T.

In some embodiments, HCDR2 may include an amino acid sequence as set forth in any one of SEQ ID NOs: 239-241.

HCDR1 of the antigen-binding protein may include amino acid sequence as set forth in SEQ ID NO: 235. For example, the sequence of HCDR1 of the antigen-binding protein may be defined by Kabat.

In some embodiments, HCDR1 may be DYYX99X100 (SEQ ID NO:235). X99 may be Lor M.

X100 may be N or H.

In some embodiments, HCDR1 may include an amino acid as set forth in any one of SEQ ID NOs: 236-237.

In some embodiments, the VH comprises HFR1, and C-terminus of HFR1 is directly or indirectly linked to the N-terminus of HCDR1. For example, the sequence of HFR1 of the antigen-binding protein may be defined by Kabat. HFR1 comprises an amino acid as set forth in SEQ ID NO: 243.

In some embodiments, HFR1 is X101IX102 X103TQSPX104 X105 X106S X107S X108G X109 X110VTITC (SEQ ID NO: 243). X101 may be A or D or E. X102 may be V or Q. X103 may be Lor M. X104 may be A or G or Por S or T. X105 may be S or T. X106 may be Lor V. X107 may be A or L. X108 may be Por V. X109 may be Dor E. X110 may be K or R.

In some embodiments, HFR1 is selected from an amino acid sequence as set forth in any one of SEQ ID NOs: 244-250.

In some embodiments, VH comprises HFR2, and HFR2 is located between HCDR1 and HCDR2. For example, the sequence of HFR2 of the antigen-binding protein may be defined by Kabat. HFR2 comprises an amino acid sequence as set forth in SEQ ID NO:251.

In some embodiments, HFR2 is WYQQKP X111 X112 X113P X114LLIY (SEQ ID NO:251). X111 may be D or G. X112 may be K or Q. X113 may be A or S. X114 may be K or Q.

In some embodiments, HFR2 is selected from an amino acid sequence as set forth in any one of SEQ ID NOs: 252-255.

In some embodiments, VH comprises HFR3, and HFR3 is located between HCDR2 and HCDR3. For example, the sequence of HFR3 of the antigen-binding protein may be defined by Kabat. HFR3 comprises an amino acid as set forth in SEQ ID NO:256.

In some embodiments, HFR3 is GVPX115RFSGSGSGTX116FTLTIX117SLX118X119ED X120ATYYC (SEQ ID NO:256). X115 may be A or D or S. X116 may be D or E or S. X117 may be S or N. X118 may be E or Q. X119 may be A or P. X120 may be A or F.

In some embodiments, HFR3 is selected from an amino acid sequence as set forth in any one of SEQ ID NOs: 257-262.

In some embodiments, VH comprises HFR4, and N-terminus of HFR4 is directly or indirectly linked to C-terminus of HCDR3. For example, the sequence of HFR4 of the antigen-binding protein may be defined by Kabat. HFR4 comprises an amino acid sequence as set forth in SEQ ID NO: 263.

In some embodiments, HFR4 is FGX121GTX122X123EX124K (SEQ ID NO:263). X121 may be A or Q. X122 may be Kor R. X123 may be Lor V. X124 may be I or L.

In some embodiments, HFR4 is selected from an amino acid sequence as set forth in any one of SEQ ID NOs: 264-268.

In some embodiments, the isolated antigen-binding protein further comprises a light chain variable region (VL), an VL comprises at least one light chain complementarity determining regions (LCDR). VL comprises LCDR1, LCDR2 and LCDR3, which is selected from an amino acid sequence as set forth in any one of SEQ ID NO: 269, SEQ ID NO:274 and SEQ ID NO:277 accordingly.

In some embodiments, VL comprises LCDR3 including an amino acid sequence as set forth in SEQ ID NO:277. For example, the sequence of LCDR3 of the antigen-binding protein may be defined by Kabat.

In some embodiments, VL comprises LCDR2 including an amino acid sequence as set forth in SEQ ID NO:274. For example, the sequence of LCDR2 of the antigen-binding protein may be defined by Kabat.

In some embodiments, LCDR2 is KTSNX125PS (SEQ ID NO:274). X125 may be L or R.

In some embodiments, LCDR2 is selected from an amino acid sequence as set forth in any one of SEQ ID NOs: 275-276.

In some embodiments, VL comprises LCDR1 including an amino acid sequence as set forth in SEQ ID NO:269. For example, the sequence of LCDR1 of the antigen-binding protein may be defined by Kabat.

In some embodiments, LCDR1 is SX126SSSIISNYX127X128 (SEQ ID NO:269). X126 may be A or T. X127 may be Lor S. X128 may be A or H.

In some embodiments, LCDR1 is selected from an amino acid sequence as set forth in any one of SEQ ID NOs: 270-273.

In some embodiments, VL comprises the LFR1, and C-terminus of the LFR1 is directly or indirectly linked to N-terminus of LCDR1. For example, the sequence of the LFR1 of the antigen-binding protein may be defined by Kabat. the LFR1 comprises an amino acid sequence as set forth in SEQ ID NO:278.

In some embodiments, the LFR1 is

X129IX130X131TQSPX132X133X134SX135SX136GX137X138VTITC (SEQ ID NO:278).

X129 may be A or E or D. X130 may be Q or V. X131 may be Lor M. X132 may be A or G or Por S or T. X133 may be S or T. X134 may be Lor V. X135 may be Lor A. X136 may be Por V. X137 may be D or E. X138 may be K or R.

In some embodiments, the LFR1 comprises an amino acid sequence as set forth in any one of SEQ ID NOs: 47, 173, 279-284.

In some embodiments, VL comprises the LFR2, and the LFR2 is located between LCDR1 and LCDR2. For example, the sequence of the LFR2 of the antigen-binding protein may be defined by Kabat, the LFR2 comprises an amino acid sequence as set forth in SEQ ID NO:284.

In some embodiments, the LFR2 is WYQQKPX139 X140 X141P X142LLIY (SEQ ID NO: 284). X139 may be D or G. X140 may be K or Q. X141 may be A or S. X142 may be K or Q.

In some embodiments, the LFR2 comprises an amino acid sequence as set forth in any one of SEQ ID NOs: 52, 285-287.

In some embodiments, VL comprises the LFR3, and the LFR3 is located between LCDR2 and LCDR3. For example, the sequence of the LFR3 of the antigen-binding protein may be defined by Kabat. The LFR3 comprises an amino acid sequence as set forth in SEQ ID NO: 288.

In some embodiments, the LFR3 is GVPX143RFSGSGSGTX144FTLTIX145SLX146X147ED X148ATYYC (SEQ ID NO:288). X143 may be A or D or S. X144 may be D or E or S. X145 may be N or S. X146 may be E or Q. X147 may be A or P. X148 may be A or F.

In some embodiments, the LFR3 comprises an amino acid sequence as set forth in any one of SEQ ID NOs: 289-293 and 57.

In some embodiments, the isolated antigen-binding protein comprises the LFR4, and N-terminus of the LFR4 is directly or indirectly linked to C terminus of LCDR3. For example, the sequence of the LFR4 of the antigen-binding protein may be defined by Kabat. The LFR4 comprises an amino acid sequence as set forth in SEQ ID NO:294.

In some embodiments, the LFR4 is FG FGX148GTX149X150EX151K (SEQ ID NO: 294). X148 may be A or Q. X149 may be K or R. X150 may be Lor V. X151 may be I or L.

In some embodiments, the LFR4 comprises an amino acid sequence as set forth in any one of SEQ ID NOs: 295-296 and 201.

In some embodiments, VH comprises sequences as set forth in SEQ ID NOs: 297-230. In some embodiments, VL comprises sequences as set forth in SEQ ID NOs: 308-317.

In some embodiments, the antigen-binding proteins described above are mentioned in Example 4, Tables 8 and 9.

In some embodiments, the distribution of antibodies for the TLA-0238 series is shown in the following table, with Table 1-5 showing the distribution of heavy chain components and Table 1-6 showing the distribution of light chain components. The technical solution of the present application includes, for the same antibody, a combination of HCDR1-3 in Table 1-5 and LCDR1-3 in Table 1-6. The technical solution of the present application includes, for the same antibody, a combination of VH in Table 1-5 and VL in Table 1-6.

TABLE 1-5

| | VH | | | | | | | | | | | | | |
|---|---|---|---|---|---|---|---|---|---|---|---|---|---|---|
| Antibody | | SEQ | | SEQ | | SEQ | | SEQ | | SEQ | | SEQ | | SEQ |
| no. | HFR1 | ID | HCDR1 | ID | HFR2 | ID | HCDR2 | ID | HFR3 | ID | HCDR3 | ID | HFR4 | ID |
| TLA0241 | | | TLA0241- | 236 | | | TLA0241- | 239 | | | TLA0241- | 242 | | |
| | | | HCDR1-1 | | | | HCDR2-1 | | | | HCDR3-1 | | | |
| TLA0241-1 | TLA0241- | 244 | TLA0241- | 236 | TLA0241- | 252 | TLA0241- | 240 | TLA0241- | 257 | TLA0241- | 242 | TLA0241- | 264 |
| | HFR1-1 | | HCDR1-1 | | HFR2-1 | | HCDR2-2 | | HFR3-1 | | HCDR3-1 | | HFR4-1 | |
| TLA0241-2 | TLA0241- | 245 | TLA0241- | 236 | TLA0241- | 252 | TLA0241- | 240 | TLA0241- | 258 | TLA0241- | 242 | TLA-0238- | 152 |
| | HFR1-2 | | HCDR1-1 | | HFR2-1 | | HCDR2-2 | | HFR3-2 | | HCDR3-1 | | HFR4-4 | |
| TLA0241-3 | TLA0241- | 246 | TLA0241 | 237 | TLA0241- | 253 | TLA0241- | 241 | TLA0241- | 259 | TLA0241- | 242 | TLA0241- | 268 |
| | HFR1-3 | | HCDR1-2 | | HFR2-2 | | HCDR2-3 | | HFR3-3 | | HCDR3-1 | | HFR4-5 | |
| TLA0241-4 | TLA0241- | 246 | TLA0241- | 237 | TLA0241- | 254 | TLA0241- | 241 | TLA0241- | 260 | TLA0241- | 242 | TLA0241- | 266 |
| | HFR1-3 | | HCDR1-2 | | HFR2-3 | | HCDR2-3 | | HFR3-4 | | HCDR3-1 | | HFR4-3 | |
| TLA0241-5 | TLA0241- | 247 | TLA0241- | 237 | TLA0241- | 254 | TLA0241- | 241 | TLA0241- | 261 | TLA0241- | 242 | TLA0241- | 268 |
| | HFR1-4 | | HCDR1-2 | | HFR2-3 | | HCDR2-3 | | HFR3-5 | | HCDR3-1 | | HFR4-5 | |
| TLA0241-6 | TLA0241- | 248 | TLA0241- | 236 | TLA0241- | 252 | TLA0241- | 240 | TLA0241- | 258 | TLA0241- | 242 | TLA-0238- | 152 |
| | HFR1-5 | | HCDR1-1 | | HFR2-1 | | HCDR2-2 | | HFR3-2 | | HCDR3-1 | | HFR4-4 | |
| TLA0241-7 | TLA0241- | 246 | TLA0241- | 237 | TLA0241- | 255 | TLA0241- | 241 | TLA0241- | 260 | TLA0241- | 242 | TLA0241- | 268 |
| | HFR1-3 | | HCDR1-2 | | HFR2-4 | | HCDR2-3 | | HFR3-4 | | HCDR3-1 | | HFR4-5 | |
| TLA0241-8 | TLA0241- | 245 | TLA0241- | 236 | TLA0241- | 252 | TLA0241- | 240 | TLA0241- | 258 | TLA0241- | 242 | TLA-0238- | 153 |
| | HFR1-2 | | HCDR1-1 | | HFR2-1 | | HCDR2-2 | | HFR3-2 | | HCDR3-1 | | HFR4-5 | |
| TLA0241-9 | TLA0241- | 249 | TLA0241- | 237 | TLA0241- | 254 | TLA0241- | 241 | TLA0241- | 260 | TLA0241- | 242 | TLA0241- | 268 |
| | HFR1-6 | | HCDR1-2 | | HFR2-3 | | HCDR2-3 | | HFR3-4 | | HCDR3-1 | | HFR4-5 | |
| TLA0241-10 | TLA0241- | 250 | TLA0241- | 237 | TLA0241- | 253 | TLA0241- | 241 | TLA0241- | 262 | TLA0241- | 242 | TLA0241- | 266 |
| | HFR1-7 | | HCDR1-2 | | HFR2-2 | | HCDR2-3 | | HFR3-6 | | HCDR3-1 | | HFR4-3 | |

TABLE 1-6

| | VL | | | | | | | | | | | | | |
|---|---|---|---|---|---|---|---|---|---|---|---|---|---|---|
| Antibody | | SEQ | | SEQ | | SEQ | | SEQ | | SEQ | | SEQ | | SEQ |
| No. | LFR1 | ID | CDR1 | ID | LFR2 | ID | LCDR2 | ID | LFR3 | ID | LCDR3 | ID | LFR4 | ID |
| TLA0241 | | | TLA0241- | 270 | | | TLA0241- | 275 | | | TLA0241- | 277 | | |
| | | | LCDR1-1 | | | | LCDR2-1 | | | | LCDR3-1 | | | |
| TLA0241-1 | TL0015- | 47 | TLA0241- | 270 | TL0015- | 52 | TLA0241- | 275 | TLA0241- | 289 | TLA0241- | 277 | TLA0238- | 201 |
| | LFR1-2/ | | LCDR1-1 | | LFR2-4/ | | LCDR2-1 | | LFR3-1 | | LCDR3-1 | | LFR4-4/ | |
| | TLA0241- | | | | TLA0241- | | | | | | | | TLA0241- | |
| | LFR1-1 | | | | LFR2-1 | | | | | | | | LFR4-1 | |
| TLA0241-2 | TLA0238- | 173 | TLA0241- | 270 | TL0015- | 52 | TLA0241- | 275 | TL0015- | 57 | TLA0241- | 277 | TLA0238- | 201 |
| | LFR1-4/ | | LCDR1-1 | | LFR2-4/ | | LCDR2-1 | | LFR3-4/ | | LCDR3-1 | | LFR4-4/ | |
| | TLA0241- | | | | TLA0241- | | | | TLA0238- | | | | TLA0241- | |
| | LFR1-2 | | | | LFR2-1 | | | | LFR3-4/ | | | | LFR4-1 | |
| | | | | | | | | | TLA0241- | | | | | |
| | | | | | | | | | LFR3-2 | | | | | |
| TLA0241-3 | TLA0241- | 279 | TLA0241- | 271 | TLA0241- | 285 | TLA0241- | 275 | TLA0241- | 290 | TLA0241- | 277 | TLA0241- | 295 |
| | LFR1-3 | | LCDR1-2 | | LFR2-2 | | LCDR2-1 | | LFR3-3 | | LCDR3-1 | | LFR4-2 | |
| TLA0241-4 | TLA0241- | 280 | TLA0241- | 271 | TLA0241- | 286 | TLA0241- | 276 | TLA0241- | 291 | TLA0241- | 277 | TLA0241- | 295 |
| | LFR1-4 | | LCDR1-2 | | LFR2-3 | | LCDR2-2 | | LFR3-4 | | LCDR3-1 | | LFR4-2 | |
| TLA0241-5 | TLA0241- | 281 | TLA0241- | 271 | TLA0241- | 287 | TLA0241- | 275 | TLA0241- | 290 | TLA0241- | 277 | TLA0241- | 296 |
| | LFR1-5 | | LCDR1-2 | | LFR2-4 | | LCDR2-1 | | LFR3-3 | | LCDR3-1 | | LFR4-3 | |
| TLA0241-6 | TLA0241- | 282 | TLA0241- | 270 | TL0015- | 52 | TLA0241- | 275 | TL0015- | 57 | TLA0241- | 277 | TLA0238- | 201 |
| | LFR1-6 | | LCDR1-1 | | LFR2-4/ | | LCDR2-1 | | LFR3-4/ | | LCDR3-1 | | LFR4-4/ | |
| | | | | | TLA0241- | | | | TLA0238- | | | | TLA0241- | |
| | | | | | LFR2-1 | | | | LFR3-4/ | | | | LFR4-1 | |
| | | | | | | | | | TLA0241- | | | | | |
| | | | | | | | | | LFR3-2 | | | | | |
| TLA0241-7 | TLA0241- | 283 | TLA0241- | 272 | TLA0241- | 285 | TLA0241- | 275 | TLA0241- | 292 | TLA0241- | 277 | TLA0241- | 295 |
| | LFR1-7 | | LCDR1-3 | | LFR2-2 | | LCDR2-1 | | LFR3-5 | | LCDR3-1 | | LFR4-2 | |
| TLA0241-8 | TLA0238- | 173 | TLA0241- | 270 | TL0015- | 52 | TLA0241- | 275 | TL0015- | 57 | TLA0241- | 277 | TLA0238- | 201 |
| | LFR1-4/ | | LCDR1-1 | | LFR2-4/ | | LCDR2-1 | | LFR3-4/ | | LCDR3-1 | | LFR4-4/ | |
| | TLA0241- | | | | TLA0241- | | | | TLA0238- | | | | TLA0241- | |
| | LFR1-2 | | | | LFR2-1 | | | | LFR3-4/ | | | | LFR4-1 | |
| | | | | | | | | | TLA0241- | | | | | |
| | | | | | | | | | LFR3-2 | | | | | |
| TLA0241-9 | TLA0241- | 279 | TLA0241- | 271 | TLA0241- | 287 | TLA0241- | 275 | TLA0241- | 290 | TLA0241- | 277 | TLA0241- | 296 |
| | LFR1-3 | | LCDR1-2 | | LFR2-4 | | LCDR2-1 | | LFR3-3 | | LCDR3-1 | | LFR4-3 | |
| TLA0241-10 | TLA0241- | 280 | TLA0241- | 273 | TLA0241- | 285 | TLA0241- | 275 | TLA0241- | 293 | TLA0241- | 277 | TLA0241- | 296 |
| | LFR1-4 | | LCDR1-4 | | LFR2-2 | | LCDR2-1 | | LFR3-6 | | LCDR3-1 | | LFR4-3 | |

### Recombinant Antibodies

In the present application, the isolated antigen-binding protein or antibody may include a heavy chain constant region, said heavy chain constant region may include a constant region derived from IgG or a constant region derived from Ig Y.

For example, the isolated antigen-binding protein or antibody may include a constant region derived from IgG. For example, the isolated antigen-binding protein or antibody may include a heavy chain. The heavy chain may include a heavy chain variable region and a heavy chain constant region. The heavy chain variable region and the heavy chain constant region may be directly or indirectly connected. For example, the heavy chain variable region may be directly connected to the heavy chain constant region. For example, the heavy chain constant region may include a constant region derived from a protein selected from the group consisting of IgG1, IgG2, IgG3, and IgG4. In some embodiments, the heavy chain constant region may include a constant region derived from a protein selected from the group consisting of human IgG1, IgG2, IgG3, and IgG4. In some embodiments, the heavy chain constant region may include a constant region derived from IgG1. The heavy chain constant region may include a constant region derived from human IgG1. In some embodiments, the constant region may include L234A and L235A mutations. In some embodiments, the constant region may include M252Y, S254T, and T256E mutations, and in some embodiments, the constant region may include L234A, L235A, M252Y, S254T, and T256E mutations. The Fc constant region of the antigen-binding protein may comprise the amino acid sequence set forth in SEQ ID NO: 320 or SEQ ID NO: 321. CH1 may comprise the amino acid sequence set forth in SEQ ID NO: 319.

In the present application, the isolated antigen-binding protein or antibody may include a light chain constant region, said light chain constant region may include a constant region derived from Igκ or a constant region derived from Igλ. For example, said light chain constant region may include a Kappa light chain constant region, such as SEQ ID NO:318.

In some embodiments, the isolated antigen-binding protein or antibody may include a light chain and a heavy chain, wherein the light chain structure is: light chain variable region VL-light chain constant region, and the heavy chain structure is: heavy chain variable region VH-CH1-Fc constant region. The VH and VL may be a combination of the VH and VL of the antibodies of the same name in Tables 1-1 and 1-2, or a combination of the antibodies of the same name in Tables 1-3 or 1-4, or a combination of the antibodies of the same name in Tables 1-5 or 1-6. The light chain constant region may have the amino acid sequence set forth in SEQ ID NO:318. CH1 may have the amino acid sequence set forth in SEQ ID NO:319. The Fc constant region may comprise the amino acid sequence set forth in SEQ ID NO:320 or SEQ ID NO:321.

For example, the isolated antigen-binding protein or antibody described herein may include the following heavy chain and light chain combinations:

Heavy chain:
(SEQ ID NO 339)
EIQLVESGGGLIQPGGSLRISCAVSGFSLSSYGVDWVRQAPGKGLEWLG
VIWGFGGTNYNSALKSRLTISKDNSKNTVYLQMNSLRAEDTAVYYCASG
NFDAMDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPK
PKDTLYITREPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPGK
Light chain:
(SEQ ID NO 340)
DIQMTQSPSSLSASVGDRVTITCRASQDISNYLNWYQQKPGKAVKLLIY
YTSRLHSGVPSRFSGSGSGTDYTLTISSLQPEDFATYFCQQGNTLPFTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

### Peptides and Immunoconjugates

In another aspect, the present application provides one or more polypeptides, which may comprise an isolated antigen-binding protein of the present application.

In another aspect, the present application provides one or more immunoconjugates, which may comprise an isolated antigen-binding protein of the present application. In some embodiments, the immunoconjugate also comprises a pharmaceutically acceptable therapeutic agent.

### Nucleic Acid, Vector, and Cell

In another aspect, the present application also provides one or more isolated nucleic acid molecules that may encode the isolated antigen-binding proteins described herein. For example, each of the one or more nucleic acid molecules may encode the entire antigen-binding protein or a portion thereof (e.g., one or more of the HCDR1-3 and the heavy chain variable regions).

The nucleic acid molecules described herein may be isolated. For example, it may be produced or synthesized by the following methods: (i) in vitro amplification, e.g., by polymerase chain reaction (PCR) amplification, (ii) clonal recombination, (iii) purification, e.g., by digestion and gel electrophoresis fractionation, or (iv) synthesis, e.g., by chemical synthesis. For example, the isolated nucleic acid may be a nucleic acid molecule prepared by recombinant DNA technology.

In the present application, the nucleic acids encoding the isolated antigen-binding proteins may be prepared by a variety of methods known in the art including, but not limited to, using reverse transcription PCR and PCR to obtain nucleic acid molecules of the isolated antigen-binding proteins described herein.

In another aspect, the present application provides one or more vectors comprising one or more nucleic acid molecules described herein. Each vector may comprise one or more of the nucleic acid molecules. In addition, the vector may also comprise other genes, such as marker genes that allow for selection of the vector in an appropriate host cell and under appropriate conditions. In addition, the vector may also comprise expression control elements that allow for the proper expression of the coding region in an appropriate host. Such control elements are well known to those skilled in the art and may include, for example, promoters, ribosome binding sites, enhancers, and other control elements that regulate gene transcription or mRNA translation, etc. In some embodiments, the expression control sequence is a regulatable element. The specific structure of the expression control sequences may vary depending on the function of the species or cell type, but typically comprise 5′ non-transcribed sequences and 5′ and 3′ non-translated sequences involved in transcription and translation initiation, respectively, such as TATA cassettes, capping sequences, CAAT sequences, etc. For example, the 5′ non-transcribed expression control sequence may comprise a promoter region, which may comprise a promoter sequence for transcription control of a functionally linked nucleic acid. The expression control sequences may also include enhancer sequences or upstream activator sequences. In the present application, suitable promoters may include, for example, promoters for SP6, T3, and T7 polymerases, human U6RNA promoters, CMV promoters, and artificial hybrid promoters thereof (e.g., CMV), wherein some portion of the promoter may be fused to some portion of the promoter of a gene for another cellular proteins (e.g., human GAPDH, glyceraldehyde-3-phosphate dehydrogenase), which may or may not comprise an additional intron. One or more nucleic acid molecules described herein can be operably linked to the expression control elements.

Such vectors may include, for example, plasmids, cosmids, viruses, phages, or other vectors commonly used in, for example, genetic engineering. For example, the vector may be an expression vector. For example, the vector may be a viral vector. The patient may be administered directly (in vivo) with the viral vector or may be administered indirectly, e.g., the patient may be administered with the cell treated with the virus in vitro (ex vivo). Viral vector technology is well known in the art and is described, for example, in Sambrook et al., (2001, Molecular Cloning: A Laboratory Manual, Cold Spring Harbor Laboratory, New York) and other virology and molecular biology manuals. Conventional virus-based systems may include retroviral vectors, lentiviral vectors, adenoviral vectors, adeno-associated viral vectors, and herpes simplex viral vectors for gene transfer. In some cases, retroviral, lentiviral, and adeno-associated viral methods can be used to transfer and integrate gene into the host genome for long term expression of the inserted gene. Lentiviral vectors are retroviral vectors capable of transducing or infecting non-dividing cells and typically producing higher viral titers. Lentiviral vectors may comprise a long terminal repeat 5′ LTR and a truncated 3′ LTR, a RRE, a rev response element (cPPT), a central termination sequence (CTS), and/or a post-translational regulatory element (WPRE). The vectors described herein can be introduced into cells.

According to another aspect, the present application provides a cell. The cell may comprise an isolated antigen-binding protein as described herein, a polypeptide as described herein, an immunoconjugate as described herein, one or more nucleic acid molecules and/or one or more vectors as described herein. For example, each or every cell may comprise a nucleic acid molecule or vector described herein. For example, each or every cell may comprise many (e.g., 2 or more) or multiple (e.g., 2 or more) kinds of nucleic acid molecules or vectors described herein. For example, the vectors described herein can be introduced into said host cells, such as prokaryotic cells (e.g., bacterial cells), CHO cells, NS/0 cells, HEK293T cells, 293F cells, or HEK293A cells, or other eukaryotic cells, such as cells from plants, fungal or yeast cells, etc. The vectors described herein can be introduced into the host cells by methods known in the art, such as electroporation, lipofectine transfection, lipofectamin transfection, etc. For example, the cells may include yeast cells. For example, the cells may include E. coli cells. For example, the cells may include mammalian cells. For example, the cells may include immune cells.

The cells may include immune cells. In some cases, the cells may include immune cells. For example, the cells may include T cells, B cells, natural killer cells (NK cells), macrophages, NKT cells, monocytes, dendritic cells, granulocytes, lymphocytes, leukocytes and/or peripheral blood mononuclear cells.

### Pharmaceutical Composition and Pharmaceutical Combination

In another aspect, the present application provides a pharmaceutical composition. The pharmaceutical composition may comprise the isolated antigen-binding protein, the polypeptide, the immunoconjugate, the isolated nucleic acid molecule, the vector, the cell, and/or the pharmaceutically acceptable adjuvant and/or excipient as described herein. In the present application, the pharmaceutically acceptable adjuvants may include buffers, antioxidants, preservatives, low molecular weight polypeptides, proteins, hydrophilic polymers, amino acids, sugars, chelating agents, counter ions, metal complexes and/or nonionic surfactants. Any conventional media or agent is contemplated for the pharmaceutical compositions of the present application, unless they are incompatible with the cells described herein. In the present application, the pharmaceutically acceptable excipients may include an additive other than main drug in the pharmaceutical preparation, which may also be referred to as pharmaceutical necessities. For example, the excipients may include binders, fillers, disintegrants, and lubricants in tablets. For example, the excipients may include alcohol, vinegar, medicinal juice, etc. in traditional Chinese medicine pills. For example, the excipients may include the base portion of semi-solid formulation ointments and creams. For example, the excipients may include preservatives, antioxidants, flavoring agents, perfuming agents, solubilizing assistant, emulsifiers, solubilizers, osmotic pressure regulators, and colorants in liquid formulations.

### Kits, Uses and Methods

In one aspect, the present application provides a method for detecting or assaying TL1A, said method may comprise using said isolated antigen-binding protein or said peptide.

In the present application, said method may comprise an in vitro method, an ex vivo method, a method for non-diagnostic or non-therapeutic purposes.

For example, said methods may comprise methods for detecting the presence and/or amount of TL1A for non-diagnostic purposes, which may comprise the following steps:
1) contacting a sample with an antigen-binding protein of the present application; and
2) detecting the presence and/or amount of said antigen-binding protein bound by the sample to determine the presence and/or expression level of TL1A in a sample obtained from a subject.

In another aspect, the present application provides a kit for TL1A, which may comprise the use of said isolated antigen-binding protein or said peptide.

In the present application, said kit may also include instructions for use, said instructions for use documenting a method for detecting the presence and/or level of TL1A. For example, said methods may include in vitro methods, ex vivo methods, methods for non-diagnostic or non-therapeutic purposes.

In another aspect, the present application provides a use of said isolated antigen-binding protein or said polypeptide in the preparation of a kit, said kit being usable for methods for detecting the presence and/or the amount of TL1A. For example, said methods may include in vitro methods, ex vivo methods, methods for non-diagnostic or non-therapeutic purposes.

In another aspect, the present application provides a method of inhibiting the binding of TL1A to DR3/TR6/DcR3 comprising administering to a subject in need thereof an effective amount of said isolated antigen-binding protein, said peptide, said immuno-fix, said isolated nucleic acid molecule, said vector, and/or said cell. Said methods may be in vitro or ex vivo methods.

In another aspect, the present application provides isolated antigen-binding proteins, said polypeptides, said immune-conjugates, said isolated nucleic acid molecules, said vectors, and said pharmaceutical compositions for use in the prevention, mitigation and/or treatment of a disease or condition.

In another aspect, in the present application, said kits and/or said pharmaceutical compositions are used for the prevention, mitigation and/or treatment of a disease or condition.

In another aspect, the present application provides the use of a pharmaceutical composition in the preparation of a drug, said drug being used for the prevention, alleviation and/or treatment of a disease or condition.

In another aspect, the present application provides a method of preventing and/or treating a disease or condition comprising administering to a subject in need thereof said isolated antigen-binding protein, said isolated nucleic acid molecule, said vector, said cell, said pharmaceutical composition.

The disease or condition disclosed herein may be an inflammatory disease, a fibrostenotic disease, or a fibrotic disease. In some instances, the disease or the condition is a TL1A-mediated disease or condition. The term, “TL1A-mediated disease or condition” refers to a disease or a condition pathology or pathogenesis that is driven, at least in part, by TL1A signaling. In some instances, the disease or the condition is immune-mediated disease or condition, such as those mediated by TL1A.

In some embodiments, the disease or the condition is an inflammatory disease or disorder that is mediated, at least in part, by TL1A signaling. Non-limiting examples of inflammatory disease include, allergy, ankylosing spondylitis, asthma, atopic dermatitis, autoimmune diseases or disorders, cancer, celiac disease, chronic obstructive pulmonary disease (COPD), chronic peptic ulcer, cystic fibrosis, diabetes (e.g., type 1 diabetes and type 2 diabetes), glomerulonephritis, gout, hepatitis (e.g., active hepatitis), an immune-mediated disease or disorder, inflammatory bowel disease (IBD) such as Crohn's disease and ulcerative colitis, myositis, osteoarthritis, pelvic inflammatory disease (PID), multiple sclerosis, neurodegenerative diseases of aging, periodontal disease (e.g., periodontitis), preperfusion injury transplant rejection, psoriasis, pulmonary fibrosis (e.g., idiopathic pulmonary fibrosis), rheumatic disease, scleroderma, sinusitis, tuberculosis.

In some embodiments, the disease or the condition is an autoimmune disease that is mediated, at least in part, by TL1A signaling. Non-limiting examples of autoimmune disease or disorder include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopeniaurpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal MotorNeuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopeni purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

In some embodiments, the disease or the condition is a cancer that is mediated, at least in part, by TL1A signaling. Non-limiting examples of cancers include Adenoid Cystic Carcinoma, Adrenal Gland Cancer, Amyloidosis, Anal Cancer, Ataxia-Telangiectasia, Atypical Mole Syndrome, Basal Cell Carcinoma, Bile Duct Cancer, Birt Hogg Dube Syndrome, Bladder Cancer, Bone Cancer, Brain Tumor, Breast Cancer, Breast Cancer in Men, Carcinoid Tumor, Cervical Cancer, Colorectal Cancer, Ductal Carcinoma, Endometrial Cancer, Esophageal Cancer, Gastric Cancer, Gastrointestinal Stromal Tumor (GIST), HER2-Positive Breast Cancer, Islet Cell Tumor, Juvenile Polyposis Syndrome, Kidney Cancer, Laryngeal Cancer, Leukemia-Acute Lymphoblastic Leukemia, Leukemia-Acute Lymphocytic (ALL), Leukemia-Acute Myeloid AML, Leukemia-Adult, Leukemia-Childhood, Leukemia-Chronic Lymphocytic (CLL), Leukemia-Chronic Myeloid (CIVIL), Liver Cancer, Lobular Carcinoma, Lung Cancer, Lung Cancer-Small Cell (SCLC), Lung Cancer-Non-small Cell (NSCLC), Lymphoma-Hodgkin's, Lymphoma-Non-Hodgkin's, Malignant Glioma, Melanoma, Meningioma, Multiple Myeloma, Myelodysplastic Syndrome (MDS), Nasopharyngeal Cancer, Neuroendocrine Tumor, Oral Cancer, Osteosarcoma, Ovarian Cancer, Pancreatic Cancer, Pancreatic Neuroendocrine Tumors, Parathyroid Cancer, Penile Cancer, Peritoneal Cancer, Peutz-Jeghers Syndrome, Pituitary Gland Tumor, Polycythemia Vera, Prostate Cancer, Renal Cell Carcinoma, Retinoblastoma, Salivary Gland Cancer, Sarcoma, Sarcoma-Kaposi, Skin Cancer, Small Intestine Cancer, Stomach Cancer, Testicular Cancer, Thymoma, Thyroid Cancer, Uterine (Endometrial) Cancer, Vaginal Cancer, and Wilms' Tumor.

In some embodiments, the disease or the condition is an inflammatory bowel disease, such as Crohn's disease (CD) or ulcerative colitis (UC). A subject may suffer from fibrosis, fibrostenosis, or a fibrotic disease, either isolated or in combination with an inflammatory disease. In some cases, the CD is severe CD. The severe CD may result from inflammation that has led to the formation of scar tissue in the intestinal wall (fibrostenosis) and/or swelling. In some cases, the severe CD is characterized by the presence of fibrotic and/or inflammatory strictures. The strictures may be determined by computed tomography enterography (CTE), and magnetic resonance imaging enterography (MRE). The disease or condition may be characterized as refractory, which in some cases, means the disease is resistant to a standard treatment (e.g., anti-TNFα therapy). Non-limiting examples of standard treatment include glucocorticosteriods, anti-TNF therapy, anti-a4-b7 therapy (vedolizumab), anti-IL12p40 therapy (ustekinumab), Thalidomide, and Cytoxin.

In the present application, the subject may include a human or non-human animal. For example, the non-human animal may be selected from the group consisting of: a monkey, a chicken, a goose, a cat, a dog, a mouse, and a rat. In addition, a non-human animal may also include any animal species other than human, such as livestock animals, or rodents, or primates, or domestic animals, or poultry animals. The human can be Caucasian, African, Asian, Sumerian, or other ethnicity, or a hybrid of various ethnicities. As another example, the human may be the elderly, adults, adolescents, children or infants.

Effective amounts in human can be extrapolated from the effective amounts in experimental animals. For example, Freireich et al. described the interrelationship of dosages for animals and humans (based on milligrams per meter squared of body surface) (Freireich et al., Cancer Chemother. Rep. 50, 219 (1966)). Body surface area can be approximately determined from the height and weight of the patient. See, e.g., Scientific Tables, Geigy Pharmaceuticals, Ardsley, N. Y, 537 (1970).

### Further Numbered Embodiments

Further numbered embodiments of the disclosure are provided below:
Embodiment 1. An isolated antigen-binding protein specifically binding to TNF-like protein A (TL1A), comprising a heavy chain variable region (VH), wherein the VH comprises at least one heavy chain complementarity-determining region (HCDR) comprising an amino acid sequence shown in SEQ ID NO:1, SEQ ID NO:5, or SEQ ID NO: 14.
Embodiment 2. The isolated antigen-binding protein of Embodiment 1, wherein the VH comprises an HCDR3 comprising an amino acid sequence as set forth in SEQ ID NO:14.
Embodiment 3. The isolated antigen-binding protein according to any one of Embodiments 1-2, wherein the HCDR3 comprises an amino acid sequence as set forth in any one SEQ ID NOs: 15-18.
Embodiment 4. The isolated antigen-binding protein according to any one of Embodiments 1-3, wherein the VH comprises an HCDR2 comprising an amino acid sequence as set forth in SEQ ID NO:5.
Embodiment 5. The isolated antigen-binding protein of Embodiment 4, wherein the HCDR2 comprises an amino acid sequence as set forth in any one of SEQ ID NOs: 6-13.
Embodiment 6. The isolated antigen-binding protein according to any one of Embodiments 1-5, wherein the VH comprises an HCDR1 which comprises an amino acid sequence as set forth in SEQ ID NO:1.
Embodiment 7. The isolated antigen-binding protein of Embodiment 6, wherein the HCDR1 comprises an amino acid sequence as set forth in any one of SEQ ID NOs: 2-4.
Embodiment 8. The isolated antigen-binding protein according to any one of Embodiments 1-7, wherein the VH comprises the HCDR1 as set forth in SEQ ID NO: 2, the HCDR2 as set forth in SEQ ID NO: 6, and the HCDR3 as set forth in SEQ ID NO: 15.
Embodiment 9. The isolated antigen-binding protein according to any one of Embodiments 1-7, wherein the VH comprises the HCDR1 as set forth in SEQ ID NO: 2, the HCDR2 as set forth in SEQ ID NO: 7, and the HCDR3 as set forth in SEQ ID NO: 15.
Embodiment 10. The isolated antigen-binding protein according to any one of Embodiments 1-7, wherein the VH comprises the HCDR1 as set forth in SEQ ID NO: 2, the HCDR2 as set forth in SEQ ID NO: 8, and the HCDR3 as set forth in SEQ ID NO: 15.
Embodiment 11. The isolated antigen-binding protein according to any one of Embodiments 1-7, wherein the VH comprises the HCDR1 as set forth in SEQ ID NO: 3, the HCDR2 as set forth in SEQ ID NO: 6, and the HCDR3 as set forth in SEQ ID NO: 15.
Embodiment 12. The isolated antigen-binding protein according to any one of Embodiments 1-7, wherein the VH comprises the HCDR1 as set forth in SEQ ID NO: 2, the HCDR2 as set forth in SEQ ID NO: 6, and the HCDR3 as set forth in SEQ ID NO: 16.
Embodiment 13. The isolated antigen-binding protein according to any one of Embodiments 1-7, wherein the VH comprises the HCDR1 as set forth in SEQ ID NO: 2, the HCDR2 as set forth in SEQ ID NO: 6, and the HCDR3 as set forth in SEQ ID NO: 17.
Embodiment 14. The isolated antigen-binding protein according to any one of Embodiments 1-7, wherein the VH comprises the HCDR1 as set forth in SEQ ID NO: 2, the HCDR2 as set forth in SEQ ID NO: 6, and the HCDR3 as set forth in SEQ ID NO: 18.
Embodiment 15. The isolated antigen-binding protein according to any one of Embodiments 1-7, wherein the VH comprises the HCDR1 as set forth in SEQ ID NO: 4, the HCDR2 as set forth in SEQ ID NO: 6, and the HCDR3 as set forth in SEQ ID NO: 15.
Embodiment 16. The isolated antigen-binding protein according to any one of Embodiments 1-7, wherein the VH comprises the HCDR1 as set forth in SEQ ID NO: 2, the HCDR2 as set forth in SEQ ID NO: 9, and the HCDR3 as set forth in SEQ ID NO: 15.
Embodiment 17. The isolated antigen-binding protein according to any one of Embodiments 1-7, wherein the VH comprises the HCDR1 as set forth in SEQ ID NO: 2, the HCDR2 as set forth in SEQ ID NO: 10, and the HCDR3 as set forth in SEQ ID NO: 15.
Embodiment 18. The isolated antigen-binding protein according to any one of Embodiments 1-7, wherein the VH comprises the HCDR1 as set forth in SEQ ID NO: 2, the HCDR2 as set forth in SEQ ID NO: 11, and the HCDR3 as set forth in SEQ ID NO: 15.
Embodiment 19. The isolated antigen-binding protein according to any one of Embodiments 1-7, wherein the VH comprises the HCDR1 as set forth in SEQ ID NO:2, the HCDR2 as set forth in SEQ ID NO: 12, and the HCDR3 as set forth in SEQ ID NO: 15.
Embodiment 20. The isolated antigen-binding protein according to any one of Embodiments 1-7, wherein the VH comprises the HCDR1 as set forth in SEQ ID NO: 2, the HCDR2 as set forth in SEQ ID NO: 13, and the HCDR3 as set forth in SEQ ID NO: 15.
Embodiment 21. The isolated antigen-binding protein according to any one of Embodiments 1-7, wherein the VH comprises the HCDR1 as set forth in SEQ ID NO: 3, the HCDR2 as set forth in SEQ ID NO: 6, and the HCDR3 as set forth in SEQ ID NO: 16.
Embodiment 22. The isolated antigen-binding protein according to any one of Embodiments 1-7, wherein the VH comprises the HCDR1 as set forth in SEQ ID NO: 3, the HCDR2 as set forth in SEQ ID NO: 6, and the HCDR3 as set forth in SEQ ID NO: 17.
Embodiment 23. The isolated antigen-binding protein according to any one of Embodiments 1-7, wherein the VH comprises the HCDR1 as set forth in SEQ ID NO: 3, the HCDR2 as set forth in SEQ ID NO: 9, and the HCDR3 as set forth in SEQ ID NO: 15.
Embodiment 24. The isolated antigen-binding protein according to any one of Embodiments 1-7, wherein the VH comprises the HCDR1 as set forth in SEQ ID NO: 3, the HCDR2 as set forth in SEQ ID NO: 10, and the HCDR3 as set forth in SEQ ID NO: 15.
Embodiment 25. The isolated antigen-binding protein according to any one of Embodiments 1-7, wherein the VH comprises the HCDR1 as set forth in SEQ ID NO: 3, the HCDR2 as set forth in SEQ ID NO: 11, and the HCDR3 as set forth in SEQ ID NO: 15.
Embodiment 26. The isolated antigen-binding protein according to any one of Embodiments 1-7, wherein the VH comprises the HCDR1 as set forth in SEQ ID NO: 2, the HCDR2 as set forth in SEQ ID NO: 9, and the HCDR3 as set forth in SEQ ID NO: 16.
Embodiment 27. The isolated antigen-binding protein according to any one of Embodiments 1-7, wherein the VH comprises the HCDR1 as set forth in SEQ ID NO: 2, the HCDR2 as set forth in SEQ ID NO: 10, and the HCDR3 as set forth in SEQ ID NO: 16.
Embodiment 28. The isolated antigen-binding protein according to any one of Embodiments 1-7, wherein the VH comprises the HCDR1 as set forth in SEQ ID NO: 2, the HCDR2 as set forth in SEQ ID NO: 11, and the HCDR3 as set forth in SEQ ID NO: 16.
Embodiment 29. The isolated antigen-binding protein according to any one of Embodiments 1-7, wherein the VH comprises the HCDR1 as set forth in SEQ ID NO: 2, the HCDR2 as set forth in SEQ ID NO: 12, and the HCDR3 as set forth in SEQ ID NO: 16.
Embodiment 30. The isolated antigen-binding protein according to any one of Embodiments 1-7, wherein the VH comprises the HCDR1 as set forth in SEQ ID NO: 2, the HCDR2 as set forth in SEQ ID NO: 10, and the HCDR3 as set forth in SEQ ID NO: 17.
Embodiment 31. The isolated antigen-binding protein according to any one of Embodiments 1-30, wherein the VH comprises an HFR1 and the HFR1 comprises an amino acid sequence of the SEQ ID NO:19.
Embodiment 32. The isolated antigen-binding protein according to Embodiment 31, wherein the HFR1 is selected from an amino acid sequence as set forth in any one of SEQ ID NO: 20-28.
Embodiment 33. The isolated antigen-binding protein according to any one of

Embodiments 1-32, wherein the VH comprises an HFR2 and the HFR2 comprises an amino acid sequence as set forth in SEQ ID NO:29.
Embodiment 34. The isolated antigen-binding protein according to any one of Embodiment 33, wherein the HFR2 is selected from an amino acid sequence as set forth in any one of SEQ ID NOs: 30-32.
Embodiment 35. The isolated antigen-binding protein according to any one of

Embodiments 1-34, wherein the VH comprises an HFR3 comprising an amino acid sequence as set forth in SEQ ID NO:33.
Embodiment 36. The isolated antigen-binding protein of Embodiment 35, wherein the HFR3 is selected from an amino acid sequence as set forth in any one of SEQ ID NOs: 34-40.
Embodiment 37. The isolated antigen-binding protein according to any one of Embodiments 1-36, wherein the VH comprises an HFR4 which comprises an amino acid sequence as set forth in SEQ ID NO:41.
Embodiment 38. The isolated antigen-binding protein according to any one of Embodiments 1-37, wherein the VH comprises the HFR1 as set forth in SEQ ID NO: 20, the HFR2 as set forth in SEQ ID NO: 30, the HFR3 as set forth in SEQ ID NO: 34, and the HFR4 as set forth in SEQ ID NO: 41.
Embodiment 39. The isolated antigen-binding protein according to any one of

Embodiments 1-37, wherein the VH comprises the HFR1 as set forth in SEQ ID NO: 21, the HFR2 as set forth in SEQ ID NO: 31, the HFR3 as set forth in SEQ ID NO: 35, and the HFR4 as set forth in SEQ ID NO: 41.
Embodiment 40. The isolated antigen-binding protein according to any one of

Embodiments 1-37, wherein the VH comprises the HFR1 as set forth in SEQ ID NO: 22, the HFR2 as set forth in SEQ ID NO: 32, the HFR3 as set forth in SEQ ID NO: 35, and the HFR4 as set forth in SEQ ID NO: 41.
Embodiment 41. The isolated antigen-binding protein according to any one of

Embodiments 1-37, wherein the VH comprises the HFR1 as set forth in SEQ ID NO: 23, the HFR2 as set forth in SEQ ID NO: 31, the HFR3 as set forth in SEQ ID NO: 36, and the HFR4 as set forth in SEQ ID NO: 41.
Embodiment 42. The isolated antigen-binding protein according to any one of Embodiments 1-37, wherein the VH comprises the HFR1 as set forth in SEQ ID NO: 23, the HFR2 as set forth in SEQ ID NO: 31, the HFR3 as set forth in SEQ ID NO: 37, and the HFR4 as set forth in SEQ ID NO: 41.
Embodiment 43. The isolated antigen-binding protein according to any one of Embodiments 1-37, wherein the VH comprises the HFR1 as set forth in SEQ ID NO: 23, the HFR2 as set forth in SEQ ID NO: 31, the HFR3 as set forth in SEQ ID NO: 35, and the HFR4 as set forth in SEQ ID NO: 41.
Embodiment 44. The isolated antigen-binding protein according to any one of Embodiments 1-37, wherein the VH comprises the HFR1 as set forth in SEQ ID NO: 24, the HFR2 as set forth in SEQ ID NO: 31, the HFR3 as set forth in SEQ ID NO: 38, and the HFR4 as set forth in SEQ ID NO: 41.
Embodiment 45. The isolated antigen-binding protein according to any one of Embodiments 1-37, wherein the VH comprises the HFR1 as set forth in SEQ ID NO: 25, the HFR2 as set forth in SEQ ID NO: 31, the HFR3 as set forth in SEQ ID NO: 37, and the HFR4 as set forth in SEQ ID NO: 41.
Embodiment 46. The isolated antigen-binding protein according to any one of Embodiments 1-37, wherein the VH comprises the HFR1 as set forth in SEQ ID NO: 25, the HFR2 as set forth in SEQ ID NO: 31, the HFR3 as set forth in SEQ ID NO: 39, and the HFR4 as set forth in SEQ ID NO: 41.
Embodiment 47. The isolated antigen-binding protein according to any one of Embodiments 1-37, wherein the VH comprises the HFR1 as set forth in SEQ ID NO: 25, the HFR2 as set forth in SEQ ID NO: 31, the HFR3 as set forth in SEQ ID NO: 40, and the HFR4 as set forth in SEQ ID NO: 41.
Embodiment 48. The isolated antigen-binding protein according to any one of Embodiments 1-37, wherein the VH comprises the HFR1 as set forth in SEQ ID NO: 26, the HFR2 as set forth in SEQ ID NO: 31, the HFR3 as set forth in SEQ ID NO: 35, and the HFR4 as set forth in SEQ ID NO: 41.
Embodiment 49. The isolated antigen-binding protein according to any one of Embodiments 1-37, wherein the VH comprises the HFR1 as set forth in SEQ ID NO: 25, the HFR2 as set forth in SEQ ID NO: 31, the HFR3 as set forth in SEQ ID NO: 35, and the HFR4 as set forth in SEQ ID NO: 41.
Embodiment 50. The isolated antigen-binding protein according to any one of Embodiments 1-37, wherein the VH comprises the HFR1 as set forth in SEQ ID NO: 27, the HFR2 as set forth in SEQ ID NO: 31, the HFR3 as set forth in SEQ ID NO: 35, and the HFR4 as set forth in SEQ ID NO: 41.
Embodiment 51. The isolated antigen-binding protein according to any one of Embodiments 1-37, wherein the VH comprises the HFR1 as set forth in SEQ ID NO: 28, the HFR2 as set forth in SEQ ID NO: 31, the HFR3 as set forth in SEQ ID NO: 35, and the HFR4 as set forth in SEQ ID NO: 41.
Embodiment 52. The isolated antigen-binding protein according to any one of Embodiments 1-51 further comprises a light chain variable region (VL) comprising at least one light chain complementarity-determining region (LCDR) comprising an amino acid sequence selected from SEQ ID NO: 42, SEQ ID NO: 43, and SEQ ID NO: 44.
Embodiment 53. The isolated antigen-binding protein according to Embodiment 52, wherein the VL comprises an LCDR3 comprising an amino acid sequence as set forth in SEQ ID NO: 44.
Embodiment 54. The isolated antigen-binding protein according to any one of Embodiments 52-53, wherein the VL comprises an LCDR2 which comprises an amino acid sequence as set forth in SEQ ID NO:43.
Embodiment 55. The isolated antigen-binding protein according to any one of Embodiments 52-54, wherein the VL comprises an LCDR1 which comprises an amino acid sequence as set forth in SEQ ID NO: 42.
Embodiment 56. The isolated antigen-binding protein according to any one of Embodiments 52-55, wherein the VL comprises an LFR1 comprising an amino acid sequence as set forth in SEQ ID NO:45.
Embodiment 57. The isolated antigen-binding protein of Embodiment 56, wherein the LFR1 comprises an amino acid sequence as set forth in any one of SEQ ID NOs: 46-47.
Embodiment 58. The isolated antigen-binding protein according to any one of Embodiments 52-57, wherein the VL comprises an LFR2 comprising an amino acid sequence as set forth in SEQ ID NO: 48.
Embodiment 59. The isolated antigen-binding protein according to Embodiment 58, wherein the LFR2 comprises an amino acid sequence as set forth in any one of SEQ ID NOS: 49-52.
Embodiment 60. The isolated antigen-binding protein according to any one of Embodiments 52-59, wherein the VL comprises an LFR3 comprising an amino acid sequence as set forth in SEQ ID NO:53.
Embodiment 61. The isolated antigen-binding protein according to Embodiment 60, wherein the LFR3 comprises an amino acid sequence as set forth in any one of SEQ ID NOs: 54-57.
Embodiment 62. The isolated antigen-binding protein according to any one of Embodiments 52-61, wherein the VL comprises an LFR4 comprising an amino acid sequence as set forth in SEQ ID NO:58.
Embodiment 63. The isolated antigen-binding protein according to Embodiment 62, wherein the LFR1 comprises an amino acid sequence as set forth in any one of SEQ ID NOs: 59-61.
Embodiment 64. The isolated antigen-binding protein according to any one of Embodiments 52-63, wherein the VL comprises the LFR1 as set forth in SEQ ID NO: 46, the LFR2 as set forth in SEQ ID NO: 49, the LFR3 as set forth in SEQ ID NO: 54, and the LFR4 as set forth in SEQ ID NO: 59.
Embodiment 65. The isolated antigen-binding protein according to any one of Embodiments 52-63, wherein the VL comprises the LFR1 as set forth in SEQ ID NO: 47, the LFR2 as set forth in SEQ ID NO: 50, the LFR3 as set forth in SEQ ID NO: 55, and the LFR4 as set forth in SEQ ID NO: 60.
Embodiment 66. The isolated antigen-binding protein according to any one of Embodiments 52-63, wherein the VL comprises the LFR1 as set forth in SEQ ID NO: 47, the LFR2 as set forth in SEQ ID NO: 51, the LFR3 as set forth in SEQ ID NO: 56, and the LFR4 as set forth in SEQ ID NO: 61.
Embodiment 67. The isolated antigen-binding protein according to any one of Embodiments 52-63, wherein the VL comprises the LFR1 as set forth in SEQ ID NO: 47, the LFR2 as set forth in SEQ ID NO: 52, the LFR3 as set forth in SEQ ID NO: 57, and the LFR4 as set forth in SEQ ID NO: 61.
Embodiment 68. The isolated antigen-binding protein according to any one of Embodiments 52-63, wherein the VL comprises the LFR1 as set forth in SEQ ID NO: 47, the LFR2 as set forth in SEQ ID NO: 52, the LFR3 as set forth in SEQ ID NO: 56, and the LFR4 as set forth in SEQ ID NO: 61.
Embodiment 69. The isolated antigen-binding protein according to any one of Embodiments 1-68, wherein the VH comprises a sequence as set forth in SEQ ID NOs: 62-99.
Embodiment 70. The isolated antigen-binding protein according to any one of Embodiments 1-69, wherein the VL comprises a sequence as set forth in SEQ ID NOs: 102-106.
Embodiment 71. An isolated antigen-binding protein specifically binding to TNF-like protein A (TL1A), comprising a heavy chain variable region (VH) comprising at least one heavy chain complementarity-determining region (HCDR) comprising an amino acid sequence selected from SEQ ID NO: 107, SEQ ID NO:115, and SEQ ID NO: 120.
Embodiment 72. The isolated antigen-binding protein of Embodiment 71, wherein the VH comprises an HCDR3 comprising an amino acid sequence as set forth in SEQ ID NO: 120.
Embodiment 73. The isolated antigen-binding protein according to any one of Embodiments 71-72, wherein the VH comprises the HCDR2 comprising an amino acid sequence as set forth in SEQ ID NO:115.
Embodiment 74. The isolated antigen-binding protein according to Embodiment 73, wherein the HCDR2 comprises an amino acid sequence as set forth in any one of SEQ ID NO: 116-119.
Embodiment 75. The isolated antigen-binding protein according to any one of Embodiments 71-74, wherein the VH comprises an HCDR1 which comprises an amino acid sequence as set forth in SEQ ID NO: 107.
Embodiment 76. The isolated antigen-binding protein according to Embodiment 75, wherein the HCDR1 comprises an amino acid sequence as set forth in any one of SEQ ID NOS: 108-114.
Embodiment 77. The isolated antigen-binding protein according to any one of Embodiments 71-76, wherein the VH comprises the HCDR1 as set forth in SEQ ID NO: 108, the HCDR2 as set forth in SEQ ID NO: 116, and the HCDR3 as set forth in SEQ ID NO: 120.
Embodiment 78. The isolated antigen-binding protein according to any one of Embodiments 71-76, wherein the VH comprises the HCDR1 as set forth in SEQ ID NO: 109, the HCDR2 as set forth in SEQ ID NO: 118, and the HCDR3 as set forth in SEQ ID NO: 120.
Embodiment 79. The isolated antigen-binding protein according to any one of Embodiments 71-76, wherein the VH comprises the HCDR1 as set forth in SEQ ID NO: 108, the HCDR2 as set forth in SEQ ID NO: 119, and the HCDR3 as set forth in SEQ ID NO: 120.
Embodiment 80. The isolated antigen-binding protein according to any one of Embodiments 71-76, wherein the VH comprises the HCDR1 as set forth in SEQ ID NO: 110, the HCDR2 as set forth in SEQ ID NO: 118, and the HCDR3 as set forth in SEQ ID NO: 120.
Embodiment 81. The isolated antigen-binding protein according to any one of Embodiments 71-76, wherein the VH comprises the HCDR1 as set forth in SEQ ID NO: 111, the HCDR2 as set forth in SEQ ID NO: 117, and the HCDR3 as set forth in SEQ ID NO: 120.
Embodiment 82. The isolated antigen-binding protein according to any one of Embodiments 71-76, wherein the VH comprises the HCDR1 as set forth in SEQ ID NO: 112, the HCDR2 as set forth in SEQ ID NO: 117, and the HCDR3 as set forth in SEQ ID NO: 120.
Embodiment 83. The isolated antigen-binding protein according to any one of Embodiments 71-76, wherein the VH comprises the HCDR1 as set forth in SEQ ID NO: 113, the HCDR2 as set forth in SEQ ID NO: 117, and the HCDR3 as set forth in SEQ ID NO: 120.
Embodiment 84. The isolated antigen-binding protein according to any one of Embodiments 71-76, wherein the VH comprises the HCDR1 as set forth in SEQ ID NO: 108, the HCDR2 as set forth in SEQ ID NO: 117, and the HCDR3 as set forth in SEQ ID NO: 120.
Embodiment 85. The isolated antigen-binding protein according to any one of Embodiments 71-76, wherein the VH comprises the HCDR1 as set forth in SEQ ID NO: 114, the HCDR2 as set forth in SEQ ID NO: 117, and the HCDR3 as set forth in SEQ ID NO: 120.
Embodiment 86. The isolated antigen-binding protein according to any one of Embodiments 71-85, wherein the VH comprises an HFR1 and the HFR1 comprises an amino acid sequence as set forth in SEQ ID NO:121.
Embodiment 87. The isolated antigen-binding protein according to Embodiment 86, wherein the HFR1 is selected from an amino acid sequence as set forth in any one of SEQ ID NOs: 122-132.
Embodiment 88. The isolated antigen-binding protein according to any one of Embodiments 71-87, wherein the VH comprises an HFR2 and the HFR2 comprises an amino acid sequence as set forth in SEQ ID NO: 133.
Embodiment 89. The isolated antigen-binding protein according to any one of Embodiment 88, wherein the HFR2 is selected from an amino acid sequence as set forth in any one of the SEQ ID NOs: 134-135, 30.
Embodiment 90. The isolated antigen-binding protein according to any one of Embodiments 71-159, wherein the VH comprises an HFR3 comprising an amino acid sequence as set forth in SEQ ID NO:136.
Embodiment 91. The isolated antigen-binding protein of Embodiment 90, wherein the HFR3 is selected from an amino acid sequence as set forth in any one of SEQ ID NOs: 137-148.
Embodiment 92. The isolated antigen-binding protein according to any one of Embodiments 71-91, wherein the VH comprises an HFR4 which comprises an amino acid sequence as set forth in SEQ ID NO: 149.
Embodiment 93. The isolated antigen-binding protein of Embodiment 92, wherein the HFR4 is selected from an amino acid sequence shown in any one of SEQ ID NOs: 150-153, 41.
Embodiment 94. The isolated antigen-binding protein according to any one of Embodiments 71-93, wherein the VH comprises the HFR1 as set forth in SEQ ID NO: 122, the HFR2 as set forth in SEQ ID NO: 134, the HFR3 as set forth in SEQ ID NO: 137, and the HFR4 as set forth in SEQ ID NO: 150.
Embodiment 95. The isolated antigen-binding protein according to any one of Embodiments 71-93, wherein the VH comprises the HFR1 as set forth in SEQ ID NO: 123, the HFR2 as set forth in SEQ ID NO: 135, the HFR3 as set forth in SEQ ID NO: 138, and the HFR4 as set forth in SEQ ID NO: 41.
Embodiment 96. The isolated antigen-binding protein according to any one of Embodiments 71-93, wherein the VH comprises the HFR1 as set forth in SEQ ID NO: 124, the HFR2 as set forth in SEQ ID NO: 135, the HFR3 as set forth in SEQ ID NO: 139, and the HFR4 as SEQ ID NO: 151.
Embodiment 97. The isolated antigen-binding protein according to any one of Embodiments 71-93, wherein the VH comprises the HFR1 as set forth in SEQ ID NO: 125, the HFR2 as set forth in SEQ ID NO: 135, the HFR3 as set forth in SEQ ID NO: 142, and the HFR4 as set forth in SEQ ID NO: 41.
Embodiment 98. The isolated antigen-binding protein according to any one of Embodiments 71-93, wherein the VH comprises the HFR1 as set forth in SEQ ID NO: 123, the HFR2 as set forth in SEQ ID NO: 135, the HFR3 as set forth in SEQ ID NO: 140, and the HFR4 as SEQ ID NO: 41.
Embodiment 99. The isolated antigen-binding protein according to any one of Embodiments 71-93, wherein the VH comprises the HFR1 as set forth in SEQ ID NO: 123, the HFR2 as set forth in SEQ ID NO: 135, the HFR3 as set forth in SEQ ID NO: 141, and the HFR4 as set forth in SEQ ID NO: 41.
Embodiment 100. The isolated antigen-binding protein according to any one of Embodiments 71-93, wherein the VH comprises the HFR1 as set forth in SEQ ID NO: 126, the HFR2 as set forth in SEQ ID NO: 135, the HFR3 as set forth in SEQ ID NO: 142, and the HFR4 as set forth in SEQ ID NO: 152.
Embodiment 101. The isolated antigen-binding protein according to any one of Embodiments 71-93, wherein the VH comprises the HFR1 as set forth in SEQ ID NO: 127, the HFR2 as set forth in SEQ ID NO: 30, the HFR3 as set forth in SEQ ID NO: 143, and the HFR4 as set forth in SEQ ID NO: 41.
Embodiment 102. The isolated antigen-binding protein according to any one of Embodiments 71-93, wherein the VH comprises the HFR1 as set forth in SEQ ID NO: 128, the HFR2 as set forth in SEQ ID NO: 30, the HFR3 as set forth in SEQ ID NO: 144, and the HFR4 as set forth in SEQ ID NO: 41.
Embodiment 103. The isolated antigen-binding protein according to any one of Embodiments 71-93, wherein the VH comprises the HFR1 as set forth in SEQ ID NO: 129, the HFR2 as set forth in SEQ ID NO: 30, the HFR3 as set forth in SEQ ID NO: 145, and the HFR4 as set forth in SEQ ID NO: 41.
Embodiment 104. The isolated antigen-binding protein according to any one of Embodiments 71-93, wherein the VH comprises the HFR1 as set forth in SEQ ID NO: 127, the HFR2 as set forth in SEQ ID NO: 30, the HFR3 as set forth in SEQ ID NO: 146, and the HFR4 as set forth in SEQ ID NO: 41.
Embodiment 105. The isolated antigen-binding protein according to any one of Embodiments 71-93, wherein the VH comprises the HFR1 as set forth in SEQ ID NO: 125, the HFR2 as set forth in SEQ ID NO: 135, the HFR3 as set forth in SEQ ID NO: 142, and the HFR4 as set forth in SEQ ID NO: 152.
Embodiment 106. The isolated antigen-binding protein according to any one of Embodiments 71-93, wherein the VH comprises the HFR1 as set forth in SEQ ID NO: 130, the HFR2 as set forth in SEQ ID NO: 30, the HFR3 as set forth in SEQ ID NO: 147, and the HFR4 as set forth in SEQ ID NO: 41.
Embodiment 107. The isolated antigen-binding protein according to any one of Embodiments 71-93, wherein the VH comprises the HFR1 as set forth in SEQ ID NO: 131, the HFR2 as set forth in SEQ ID NO: 135, the HFR3 as set forth in SEQ ID NO: 148, and the HFR4 as set forth in SEQ ID NO: 153.
Embodiment 108. The isolated antigen-binding protein according to any one of Embodiments 71-93, wherein the VH comprises the HFR1 as set forth in SEQ ID NO: 132, the HFR2 as set forth in SEQ ID NO: 30, the HFR3 as set forth in SEQ ID NO: 145, and the HFR4 as set forth in SEQ ID NO: 41.
Embodiment 109. The isolated antigen-binding protein according to any one of Embodiments 71-108 further comprises a light chain variable region (VL) comprising at least one light chain complementarity-determining region (LCDR) including an amino acid sequence selected from SEQ ID NO: 154, SEQ ID NO: 159, and SEQ ID NO: 163.
Embodiment 110. The isolated antigen-binding protein according to any one of Embodiments 71-109, wherein the VL comprises an LCDR3 comprising an amino acid sequence as set forth in SEQ ID NO:163.
Embodiment 111. The isolated antigen-binding protein of Embodiment 110, wherein the LCDR3 comprises an amino acid sequence as set forth in any one of SEQ ID NOs: 164-168.
Embodiment 112. The isolated antigen-binding protein according to any one of Embodiments 71-111, wherein the VL comprises an LCDR2 comprising an amino acid sequence as set forth in SEQ ID NO: 159.
Embodiment 113. The isolated antigen-binding protein according to Embodiment 112, wherein the LCDR2 comprises an amino acid sequence as set forth in any one of the SEQ ID NOs: 160-162.
Embodiment 114. The isolated antigen-binding protein according to any one of Embodiments 71-113, wherein the VL comprises an LCDR1 comprising an amino acid sequence as set forth in SEQ ID NO:154.
Embodiment 115. The isolated antigen-binding protein according to Embodiment 114, wherein the LCDR1 comprises an amino acid sequence as set forth in any one of SEQ ID NOS: 155-158.
Embodiment 116. The isolated antigen-binding protein according to any one of Embodiments 71-116, wherein the VL comprises the LCDR1 as set forth in SEQ ID NO: 157, the LCDR2 as set forth in SEQ ID NO: 161, and the LCDR3 as set forth in SEQ ID NO: 164.
Embodiment 117. The isolated antigen-binding protein according to any one of Embodiments 71-116, wherein the VL comprises the LCDR1 as set forth in SEQ ID NO: 157, the LCDR2 as set forth in SEQ ID NO: 162, and the LCDR3 as set forth in SEQ ID NO: 164.
Embodiment 118. The isolated antigen-binding protein according to any one of Embodiments 71-116, wherein the VL comprises the LCDR1 as set forth in SEQ ID NO: 156, the LCDR2 as set forth in SEQ ID NO: 161, and the LCDR3 as set forth in SEQ ID NO: 164.
Embodiment 119. The isolated antigen-binding protein according to any one of Embodiments 71-116, wherein the VL comprises the LCDR1 as set forth in SEQ ID NO: 158, the LCDR2 as set forth in SEQ ID NO: 160, and the LCDR3 as set forth in SEQ ID NO: 164.
Embodiment 120. The isolated antigen-binding protein according to any one of Embodiments 71-116, wherein the VL comprises the LCDR1 as set forth in SEQ ID NO: 155, the LCDR2 as set forth in SEQ ID NO: 160, and the LCDR3 as set forth in SEQ ID NO: 164.
Embodiment 121. The isolated antigen-binding protein according to any one of Embodiments 71-116, wherein the VL comprises the LCDR1 as set forth in SEQ ID NO: 156, the LCDR2 as set forth in SEQ ID NO: 162, and the LCDR3 as set forth in SEQ ID NO: 164.
Embodiment 122. The isolated antigen-binding protein according to any one of Embodiments 71-116, wherein the VL comprises the LCDR1 as set forth in SEQ ID NO: 155, the LCDR2 as set forth in SEQ ID NO: 160, and the LCDR3 as set forth in SEQ ID NO: 165.
Embodiment 123. The isolated antigen-binding protein according to any one of Embodiments 71-116, wherein the VL comprises the LCDR1 as set forth in SEQ ID NO: 155, the LCDR2 as set forth in SEQ ID NO: 160, and the LCDR3 as set forth in SEQ ID NO: 166.
Embodiment 124. The isolated antigen-binding protein according to any one of Embodiments 71-116, wherein the VL comprises the LCDR1 as set forth in SEQ ID NO: 155, the LCDR2 as set forth in SEQ ID NO: 160, and the LCDR3 as set forth in SEQ ID NO: 167.
Embodiment 125. The isolated antigen-binding protein according to any one of Embodiments 71-116, wherein the VL comprises the LCDR1 as set forth in SEQ ID NO: 155, the LCDR2 as set forth in SEQ ID NO: 160, and the LCDR3 as set forth in SEQ ID NO: 168.
Embodiment 126. The isolated antigen-binding protein according to any one of Embodiments 71-125, wherein the VL comprises an LFR1 including an amino acid sequence as set forth in SEQ ID NO:169.
Embodiment 127. The Isolated antigen-binding protein according to Embodiment 126, wherein the LFR1 comprises an amino acid sequence as set forth in any one of SEQ ID NOs: 170-179.
Embodiment 128. The isolated antigen-binding protein according to any one of Embodiments 71-127, wherein the VL comprises an LFR2 comprising an amino acid sequence as set forth in SEQ ID NO: 180.
Embodiment 129. The isolated antigen-binding protein of Embodiment 128, wherein the LFR2 comprises an amino acid sequence as set forth in any one of SEQ ID NOs: 181-188.
Embodiment 130. The isolated antigen-binding protein according to any one of Embodiments 71-129, wherein the VL comprises an LFR3 comprising an amino acid sequence as set forth in SEQ ID NO: 189.
Embodiment 131. The isolated antigen-binding protein of Embodiment 130, wherein the LFR3 comprises an amino acid sequence as set forth in any one of SEQ ID NOs: 190-197, 57.
Embodiment 132. The isolated antigen-binding protein according to any one of Embodiments 71-131, wherein the VL comprises an LFR4 comprising an amino acid sequence as set forth in SEQ ID NO: 198.
Embodiment 133. The isolated antigen-binding protein of Embodiment 132, wherein the LFR4 comprises an amino acid sequence as set forth in any one of SEQ ID NOs: 199-201, 61.
Embodiment 134. The isolated antigen-binding protein according to any one of Embodiments 71-133, wherein the VL comprises the LFR1 as set forth in SEQ ID NO: 170, the LFR2 as set forth in SEQ ID NO: 181, the LFR3 as set forth in SEQ ID NO: 190, and the LFR4 as set forth in SEQ ID NO: 199.
Embodiment 135. The isolated antigen-binding protein according to any one of Embodiments 71-133, wherein the VL comprises the LFR1 as set forth in SEQ ID NO: 171, the LFR2 as set forth in SEQ ID NO: 182, the LFR3 as set forth in SEQ ID NO: 191, and the LFR4 as set forth in SEQ ID NO: 200.
Embodiment 136. The isolated antigen-binding protein according to any one of Embodiments 71-133, wherein the VL comprises the LFR1 as set forth in SEQ ID NO: 172, the LFR2 as set forth in SEQ ID NO: 183, the LFR3 as set forth in SEQ ID NO: 192, and the LFR4 as set forth in SEQ ID NO: 61.
Embodiment 137. The isolated antigen-binding protein according to any one of Embodiments 71-133, wherein the VL comprises the LFR1 as set forth in SEQ ID NO: 174, the LFR2 as set forth in SEQ ID NO: 184, the LFR3 as set forth in SEQ ID NO: 57, and the LFR4 as set forth in SEQ ID NO: 199.
Embodiment 138. The isolated antigen-binding protein according to any one of Embodiments 71-133, wherein the VL comprises the LFR1 as set forth in SEQ ID NO: 175, the LFR2 as set forth in SEQ ID NO: 185, the LFR3 as set forth in SEQ ID NO: 193, and the LFR4 as set forth in SEQ ID NO: 199.
Embodiment 139. The isolated antigen-binding protein according to any one of Embodiments 71-133, wherein the VL comprises the LFR1 as set forth in SEQ ID NO: 176, the LFR2 as set forth in SEQ ID NO: 186, the LFR3 as set forth in SEQ ID NO: 194, and the LFR4 as set forth in SEQ ID NO: 199.
Embodiment 140. The isolated antigen-binding protein according to any one of Embodiments 71-133, wherein the VL comprises the LFR1 as set forth in SEQ ID NO: 177, the LFR2 as set forth in SEQ ID NO: 187, the LFR3 as set forth in SEQ ID NO: 195, and the LFR4 as set forth in SEQ ID NO: 199.
Embodiment 141. The isolated antigen-binding protein according to any one of Embodiments 71-133, wherein the VL comprises the LFR1 as set forth in SEQ ID NO: 178, the LFR2 as set forth in SEQ ID NO: 188, the LFR3 as set forth in SEQ ID NO: 196, and the LFR4 as set forth in SEQ ID NO: 199.
Embodiment 142. The isolated antigen-binding protein according to any one of Embodiments 71-133, wherein the VL comprises the LFR1 as set forth in SEQ ID NO: 179, the LFR2 as set forth in SEQ ID NO: 187, the LFR3 as set forth in SEQ ID NO: 195, and the LFR4 as set forth in SEQ ID NO: 199.
Embodiment 143. The isolated antigen-binding protein according to any one of Embodiments 71-133, wherein the VL comprises the LFR1 as set forth in SEQ ID NO: 173, the LFR2 as set forth in SEQ ID NO: 183, the LFR3 as set forth in SEQ ID NO: 192, and the LFR4 as set forth in SEQ ID NO: 201.
Embodiment 144. The isolated antigen-binding protein according to any one of Embodiments 71-143, wherein the VH comprises a sequence as set forth in SEQ ID NOs: 202-220, 332-334.
Embodiment 145. The isolated antigen-binding protein according to any one of Embodiments 71-144, wherein the VL comprises a sequence as set forth in SEQ ID NOs: 221-234, 335-338.
Embodiment 146. An isolated antigen-binding protein capable of specifically binding to TNF-like protein A (TL1A), comprising a heavy chain variable region (VH) comprising at least one heavy chain complementarity-determining region (HCDR) comprising an amino acid sequence selected from an amino acid sequence as set forth in SEQ ID NO:242, SEQ ID NO: 238, and SEQ ID NO:235.
Embodiment 147. The isolated antigen-binding protein of Embodiment 146, wherein the VH comprises an HCDR3 comprising an amino acid sequence as set forth in SEQ ID NO: 242.
Embodiment 148. The isolated antigen-binding protein according to any one of Embodiments 146-147, wherein the VH comprises an HCDR2 comprising an amino acid sequence as set forth in SEQ ID NO:238.
Embodiment 149. The isolated antigen-binding protein according to Embodiment 218, wherein the HCDR2 comprises an amino acid sequence as set forth in any one of the SEQ ID NOs: 239-241.
Embodiment 150. The isolated antigen-binding protein according to any one of Embodiments 146-149, wherein the VH comprises an HCDR1 comprising an amino acid sequence as set forth in SEQ ID NO:235.
Embodiment 151. The isolated antigen-binding protein of Embodiment 220, wherein the HCDR1 comprises an amino acid sequence as set forth in any one of SEQ ID NOs: 236-237.
Embodiment 152. The isolated antigen-binding protein according to any one of Embodiments 146-151, wherein the VH comprises the HCDR1 as set forth in SEQ ID NO: 108, the HCDR2 as set forth in SEQ ID NO: 116, and the HCDR3 as set forth in SEQ ID NO: 120.
Embodiment 153. The isolated antigen-binding protein according to any one of Embodiments 146-152, wherein the VH comprises the HCDR1 as set forth in SEQ ID NO: 236, the HCDR2 as set forth in SEQ ID NO: 239, and the HCDR3 as set forth in SEQ ID NO: 242.
Embodiment 154. The isolated antigen-binding protein according to any one of Embodiments 146-152, wherein the VH comprises the HCDR1 as set forth in SEQ ID NO: 236, the HCDR2 as set forth in SEQ ID NO: 240, and the HCDR3 as set forth in SEQ ID NO: 242.
Embodiment 155. The isolated antigen-binding protein according to any one of Embodiments 146-152, wherein the VH comprises the HCDR1 as set forth in SEQ ID NO: 237, the HCDR2 as set forth in SEQ ID NO: 241, and the HCDR3 as set forth in SEQ ID NO: 242.
Embodiment 156. The isolated antigen-binding protein according to any one of Embodiments 146-155, wherein the VH comprises an HFR1 comprising an amino acid sequence as set forth in SEQ ID NO:243.
Embodiment 157. The isolated antigen-binding protein of Embodiment 156, wherein the HFR1 is selected from an amino acid sequence as set forth in any one of SEQ ID NOs: 244-250.
Embodiment 158. The isolated antigen-binding protein according to any one of Embodiments 146-157, wherein the VH comprises the HFR2 and the HFR2 comprises an amino acid sequence as set forth in SEQ ID NO:251.
Embodiment 159. The isolated antigen-binding protein according to any one of Embodiment 158, wherein the HFR2 is selected from an amino acid sequence as set forth in any one of SEQ ID NOs: 252-255.
Embodiment 160. The isolated antigen-binding protein according to any one of Embodiments 146-159, wherein the VH comprises the HFR3 comprising an amino acid sequence as set forth in SEQ ID NO:256.
Embodiment 161. The isolated antigen-binding protein of Embodiment 160, wherein the HFR3 is selected from an amino acid sequence as set forth in any one of SEQ ID NOs: 257-262.
Embodiment 162. The isolated antigen-binding protein according to any one of Embodiments 146-161, wherein the VH comprises the HFR4 comprising an amino acid sequence as set forth in SEQ ID NO:263.
Embodiment 163. The isolated antigen-binding protein according to Embodiment 162, wherein the HFR4 is selected from an amino acid sequence as set forth in any one of SEQ ID NOs: 264-268.
Embodiment 164. The isolated antigen-binding protein according to any one of Embodiments 146-163, wherein the VH comprises the HFR1 as set forth in SEQ ID NO: 244, the HFR2 as set forth in SEQ ID NO: 252, the HFR3 as set forth in SEQ ID NO: 257, and the HFR4 as set forth in SEQ ID NO: 264.
Embodiment 165. The isolated antigen-binding protein according to any one of Embodiments 146-163, wherein the VH comprises the HFR1 as set forth in SEQ ID NO: 245, the HFR2 as set forth in SEQ ID NO: 252, the HFR3 as set forth in SEQ ID NO: 258, and the HFR4 as set forth in SEQ ID NO: 265.
Embodiment 166. The isolated antigen-binding protein according to any one of Embodiments 146-163, wherein the VH comprises the HFR1 as set forth in SEQ ID NO: 246, the HFR2 as set forth in SEQ ID NO: 253, the HFR3 as set forth in SEQ ID NO: 259, and the HFR4 as set forth in SEQ ID NO: 268.
Embodiment 167. The isolated antigen-binding protein according to any one of Embodiments 146-163, wherein the VH comprises the HFR1 as set forth in SEQ ID NO: 246, the HFR2 as set forth in SEQ ID NO: 254, the HFR3 as set forth in SEQ ID NO: 260, and the HFR4 as set forth in SEQ ID NO: 266.
Embodiment 168. The isolated antigen-binding protein according to any one of Embodiments 146-163, wherein the VH comprises the HFR1 as set forth in SEQ ID NO: 247, the HFR2 as set forth in SEQ ID NO: 254, the HFR3 as set forth in SEQ ID NO: 261, and the HFR4 as set forth in SEQ ID NO: 268.
Embodiment 169. The isolated antigen-binding protein according to any one of Embodiments 146-163, wherein the VH comprises the HFR1 as set forth in SEQ ID NO: 248, the HFR2 as set forth in SEQ ID NO: 252, the HFR3 as set forth in SEQ ID NO: 258, and the HFR4 as set forth in SEQ ID NO: 265.
Embodiment 170. The isolated antigen-binding protein according to any one of Embodiments 146-163, wherein the VH comprises the HFR1 as set forth in SEQ ID NO: 246, the HFR2 as set forth in SEQ ID NO: 255, the HFR3 as set forth in SEQ ID NO: 260, and the HFR4 as set forth in SEQ ID NO: 268.
Embodiment 171. The isolated antigen-binding protein according to any one of Embodiments 146-163, wherein the VH comprises the HFR1 as set forth in SEQ ID NO: 245, the HFR2 as set forth in SEQ ID NO: 252, the HFR3 as set forth in SEQ ID NO: 258, and the HFR4 as set forth in SEQ ID NO: 267.
Embodiment 172. The isolated antigen-binding protein according to any one of Embodiments 146-163, wherein the VH comprises the HFR1 as set forth in SEQ ID NO: 249, the HFR2 as set forth in SEQ ID NO: 254, the HFR3 as set forth in SEQ ID NO: 260, and the HFR4 as set forth in SEQ ID NO: 268.
Embodiment 173. The isolated antigen-binding protein according to any one of Embodiments 146-163, wherein the VH comprises the HFR1 as set forth in SEQ ID NO: 250, the HFR2 as set forth in SEQ ID NO: 253, the HFR3 as set forth in SEQ ID NO: 262, and the HFR4 as set forth in SEQ ID NO: 266.
Embodiment 174. The isolated antigen-binding protein as described in any one of Embodiments 146-173 further comprises a light chain variable region (VL) comprising at least one light chain complementarity-determining region (LCDR) comprising an amino acid sequence selected from SEQ ID NO: 269, SEQ ID NO: 274, and SEQ ID NO: 277.
Embodiment 175. The isolated antigen-binding protein according to any one of Embodiments 146-174, wherein the VL comprises an LCDR3 comprising an amino acid sequence as set forth in SEQ ID NO:277.
Embodiment 176. The isolated antigen-binding protein according to any one of Embodiments 146-175, wherein the VL comprises an LCDR2 comprising an amino acid sequence as set forth in SEQ ID NO:274.
Embodiment 177. The isolated antigen-binding protein according to Embodiment 176, wherein the LCDR2 comprises an amino acid sequence as set forth in any one of SEQ ID NOS: 275-276.
Embodiment 178. The isolated antigen-binding protein according to any one of Embodiments 146-177, wherein the VL comprises an LCDR1 comprising an amino acid sequence as set forth in SEQ ID NO:269.
Embodiment 179. The isolated antigen-binding protein according to Embodiment 178, wherein the LCDR1 comprises an amino acid sequence as set forth in any one of the SEQ ID NOs: 270-273.
Embodiment 180. The isolated antigen-binding protein according to any one of Embodiments 146-179, wherein the VL comprises the LCDR1 as set forth in SEQ ID NO: 270, the LCDR2 as set forth in SEQ ID NO: 275, and the LCDR3 as set forth in SEQ ID NO: 277.
Embodiment 181. The isolated antigen-binding protein according to any one of Embodiments 146-179, wherein the VL comprises the LCDR1 as set forth in SEQ ID NO: 271, the LCDR2 as set forth in SEQ ID NO: 275, and the LCDR3 as set forth in SEQ ID NO: 277.
Embodiment 182. The isolated antigen-binding protein according to any one of Embodiments 146-179, wherein the VL comprises the LCDR1 as set forth in SEQ ID NO: 272, the LCDR2 as set forth in SEQ ID NO: 275, and the LCDR3 as set forth in SEQ ID NO: 277.
Embodiment 183. The isolated antigen-binding protein according to any one of Embodiments 146-179, wherein the VL comprises the LCDR1 as set forth in SEQ ID NO: 273, the LCDR2 as set forth in SEQ ID NO: 275, and the LCDR3 as set forth in SEQ ID NO: 277.
Embodiment 184. The isolated antigen-binding protein according to any one of Embodiments 146-179, wherein the VL comprises the LCDR1 as set forth in SEQ ID NO: 271, the LCDR2 as set forth in SEQ ID NO: 276, and the LCDR3 as set forth in SEQ ID NO: 277.
Embodiment 185. The isolated antigen-binding protein according to any one of Embodiments 146-184, wherein the VL comprises an LFR1 comprising an amino acid sequence as set forth in SEQ ID NO:278.
Embodiment 186. The isolated antigen-binding protein according to Embodiment 185, wherein the LFR1 comprises an amino acid sequence as set forth in any one of SEQ ID NOS: 47, 173, 279-283.
Embodiment 187. The isolated antigen-binding protein according to any one of Embodiments 146-186, wherein the VL comprises an LFR2 comprising an amino acid sequence as set forth in SEQ ID NO:284.
Embodiment 188. The isolated antigen-binding protein of Embodiment 187, wherein the LFR2 comprises an amino acid sequence as set forth in any one of SEQ ID NOs: 52, 285-287.
Embodiment 189. The isolated antigen-binding protein according to any one of Embodiments 146-188, wherein the VL comprises an LFR3 which comprises an amino acid sequence as set forth in SEQ ID NO: 288.
Embodiment 190. The isolated antigen-binding protein of Embodiment 189, wherein the LFR3 comprises an amino acid sequence as set forth in any one of SEQ ID NOs: 289-293, 57.
Embodiment 191. The isolated antigen-binding protein according to any one of Embodiments 146-190, wherein the VL comprises an LFR4 comprising an amino acid sequence as set forth in SEQ ID NO:294.
Embodiment 192. The isolated antigen-binding protein of Embodiment 191, wherein the LFR4 comprises an amino acid sequence as set forth in any one of SEQ ID NOs: 201, 295-296.
Embodiment 193. The isolated antigen-binding protein according to any one of Embodiments 146-192, wherein the VL comprises the LFR1 as set forth in SEQ ID NO: 170, the LFR2 as set forth in SEQ ID NO: 181, the LFR3 as set forth in SEQ ID NO: 190, and the LFR4 as set forth in SEQ ID NO: 199.
Embodiment 194. The isolated antigen-binding protein according to any one of Embodiments 146-192, wherein the VL comprises the LFR1 as set forth in SEQ ID NO: 171, the LFR2 as set forth in SEQ ID NO: 182, the LFR3 as set forth in SEQ ID NO: 191, and the LFR4 as set forth in SEQ ID NO: 200.
Embodiment 195. The isolated antigen-binding protein according to any one of Embodiments 146-192, wherein the VL comprises the LFR1 as set forth in SEQ ID NO: 172, the LFR2 as set forth in SEQ ID NO: 183, the LFR3 as set forth in SEQ ID NO: 192, and the LFR4 as set forth in SEQ ID NO: 61.
Embodiment 196. The isolated antigen-binding protein according to any one of Embodiments 146-192, wherein the VL comprises the LFR1 as set forth in SEQ ID NO: 174, the LFR2 as set forth in SEQ ID NO: 184, the LFR3 as set forth in SEQ ID NO: 57, and the LFR4 as set forth in SEQ ID NO: 199.
Embodiment 197. The isolated antigen-binding protein according to any one of Embodiments 146-192, wherein the VL comprises the LFR1 as set forth in SEQ ID NO: 175, the LFR2 as set forth in SEQ ID NO: 185, the LFR3 as set forth in SEQ ID NO: 193, and the LFR4 as set forth in SEQ ID NO: 199.
Embodiment 198. The isolated antigen-binding protein according to any one of Embodiments 146-192, wherein the VL comprises the LFR1 as set forth in SEQ ID NO: 176, the LFR2 as set forth in SEQ ID NO: 186, the LFR3 as set forth in SEQ ID NO: 194, and the LFR4 as set forth in SEQ ID NO: 199.
Embodiment 199. The isolated antigen-binding protein according to any one of Embodiments 146-192, wherein the VL comprises the LFR1 as set forth in SEQ ID NO: 177, the LFR2 as set forth in SEQ ID NO: 187, the LFR3 as set forth in SEQ ID NO: 195, and the LFR4 as set forth in SEQ ID NO: 199.
Embodiment 200. The isolated antigen-binding protein according to any one of Embodiments 146-192, wherein the VL comprises the LFR1 as set forth in SEQ ID NO: 178, the LFR2 as set forth in SEQ ID NO: 188, the LFR3 as set forth in SEQ ID NO: 196, and the LFR4 as set forth in SEQ ID NO: 199.
Embodiment 201. The isolated antigen-binding protein according to any one of Embodiments 146-192, wherein the VL comprises the LFR1 as set forth in SEQ ID NO: 179, the LFR2 as set forth in SEQ ID NO: 187, the LFR3 as set forth in SEQ ID NO: 195, and the LFR4 as set forth in SEQ ID NO: 199.
Embodiment 202. The isolated antigen-binding protein according to any one of Embodiments 146-192, wherein the VL comprises the LFR1 as set forth in SEQ ID NO: 173, the LFR2 as set forth in SEQ ID NO: 183, the LFR3 as set forth in SEQ ID NO: 192, and the LFR4 as set forth in SEQ ID NO: 201.
Embodiment 203. The isolated antigen-binding protein according to any one of Embodiments 146-202, wherein the VH comprises a sequence as set forth in SEQ ID NOs: 202-220, 332-334.
Embodiment 204. The isolated antigen-binding protein according to any one of Embodiments 146-203, wherein the VL comprises a sequence as set forth in SEQ ID NOs: 221-234, 335-338.
Embodiment 205. The isolated antigen-binding protein according to any one of Embodiments 1-204, further comprises a heavy chain constant region of IgG and/or a light chain constant region of a human antibody.
Embodiment 206. The isolated antigen-binding protein according to any one of Embodiments 1-205, comprising a heavy chain constant region derived from IgG1, IgG2, IgG3, or IgG4.
Embodiment 207. The isolated antigen-binding protein according to any one of Embodiments 1-206 comprising a Kappa or Lambda light chain constant region.
Embodiment 208. The isolated antigen-binding protein according to any one of Embodiments 1-207, wherein the heavy chain constant region is derived from IgG, IgM, IgA, IgD, or IgE.
Embodiment 209. An isolated nucleic acid molecule encoding an isolated antigen-binding protein as described in any one of Embodiments 1-208.
Embodiment 210. An expression vector comprising nucleic acids according to Embodiment 209.
Embodiment 211. A host cell comprising nucleic acids isolated according to Embodiment 209 and/or the expression vector according to Embodiment 210.
Embodiment 212. A composition comprising the isolated antigen-binding protein according to any one of Embodiments 1-208 and an optionally pharmaceutical acceptable carrier.
Embodiment 213. A preparation method for isolated antigen-binding proteins for TL1A, comprising: culturing the host cells of Embodiment 211, and recovering the isolated antigen-binding proteins.
Embodiment 214. A method for treating TL1A-mediated disease or condition, which comprises:
1. administering a subject in need thereof the isolated antigen-binding protein according to any one of Embodiments 1-208, the isolated nucleic acid according to Embodiment 209, and/or the expression vector according to Embodiment 210, the host cell according to any one of Embodiment 211, and/or the composition according to Embodiment 212.
Embodiment 215. The isolated antigen-binding protein according to any one of Embodiments 1-208, the nucleic acid isolated according to Embodiment 209, the expression vector according to Embodiment 210, the host cell according to any one of Embodiment 211, and/or the composition according to Embodiment 212, which is used for the treatment of TL1A-mediated disease or condition.
Embodiment 216. Use of the Isolated antigen-binding proteins as described in any one of Embodiments 1-208, isolated nucleic acids according to Embodiment 209, and/or expression vectors according to Embodiment 210, host cells according to any one of Embodiment 211, or composition according to Embodiment 212 for the treatment of TL1A-mediated disease or condition.
Embodiment 217. Use of the isolated antigen-binding protein of Embodiments 1-208, the isolated nucleic acid according to Embodiment 209, the expression vector according to Embodiment 210, the host cell according to any one of Embodiment 211, and/or the composition according to Embodiment 212 in preparation a drug for the treatment of TL1A-mediated disease or condition.
Embodiment 218. The method as described in Embodiment 214, or the use as described in Embodiments 215-217, wherein the TL1A-mediated disease or condition is inflammatory disease.
Embodiment 219. The method or use as described in Embodiment 218, wherein the TL1A-mediated inflammatory disease is selected from one of the following: allergy, ankylosing spondylitis, asthma, atopic dermatitis, autoimmune diseases or disorders, cancer, celiac disease, chronic obstructive pulmonary disease (COPD), chronic peptic ulcer, cystic fibrosis, diabetes (e.g., type 1 diabetes and type 2 diabetes), glomerulonephritis, gout, hepatitis (e.g., active hepatitis), an immune-mediated disease or disorder, inflammatory bowel disease (IBD) such as Crohn's disease and ulcerative colitis, myositis, osteoarthritis, pelvic inflammatory disease (PID), multiple sclerosis, neurodegenerative diseases of aging, periodontal disease (e.g., periodontitis), preperfusion injury transplant rejection, psoriasis, pulmonary fibrosis (e.g., idiopathic pulmonary fibrosis), rheumatic disease, scleroderma, sinusitis, tuberculosis.
Embodiment 220. The method or use of Embodiment 219, wherein inflammatory bowel disease (IBD) comprises: Ulcerative colitis (UC) or Crohn's disease (CD).
Embodiment 221. The method as described in Embodiment 214, or the use as described in Embodiments 215-217, wherein the TL1A-mediated disease or condition are autoimmune diseases.
Embodiment 222. The method or use as described in Embodiment 221, wherein the TL1A-mediated autoimmune diseases are selected from one of the following: Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Bal disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal MotorNeuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.
Embodiment 223. The method as described in Embodiment 214, or the use as described in Embodiments 215-217, wherein the TL1A-mediated disease or condition is cancer.
Embodiment 224. The method as described in Embodiment 223, or the use described in Embodiments 215-217, wherein TL1A-mediated cancers are selected from one or more of the following: Adenoid Cystic Carcinoma, Adrenal Gland Cancer, Amyloidosis, Anal Cancer, Ataxia-Telangiectasia, Atypical Mole Syndrome, Basal Cell Carcinoma, Bile Duct Cancer, Birt Hogg Dube Syndrome, Bladder Cancer, Bone Cancer, Brain Tumor, Breast Cancer, Breast Cancer in Men, Carcinoid Tumor, Cervical Cancer, Colorectal Cancer, Ductal Carcinoma, Endometrial Cancer, Esophageal Cancer, Gastric Cancer, Gastrointestinal Stromal Tumor (GIST), HER2-Positive Breast Cancer, Islet Cell Tumor, Juvenile Polyposis Syndrome, Kidney Cancer, Laryngeal Cancer, Leukemia-Acute Lymphoblastic Leukemia, Leukemia-Acute Lymphocytic (ALL), Leukemia-Acute Myeloid AML, Leukemia-Adult, Leukemia—Childhood, Leukemia-Chronic Lymphocytic (CLL), Leukemia-Chronic Myeloid (CIVIL), Liver Cancer, Lobular Carcinoma, Lung Cancer, Lung Cancer-Small Cell (SCLC), Lung Cancer-Non-small Cell (NSCLC), Lymphoma-Hodgkin's, Lymphoma-Non-Hodgkin's, Malignant Glioma, Melanoma, Meningioma, Multiple Myeloma, Myelodysplastic Syndrome (MDS), Nasopharyngeal Cancer, Neuroendocrine Tumor, Oral Cancer, Osteosarcoma, Ovarian Cancer, Pancreatic Cancer, Pancreatic Neuroendocrine Tumors, Parathyroid Cancer, Penile Cancer, Peritoneal Cancer, Peutz-Jeghers Syndrome, Pituitary Gland Tumor, Polycythemia Vera, Prostate Cancer, Renal Cell Carcinoma, Retinoblastoma, Salivary Gland Cancer, Sarcoma, Sarcoma-Kaposi, Skin Cancer, Small Intestine Cancer, Stomach Cancer, Testicular Cancer, Thymoma, Thyroid Cancer, Uterine (Endometrial) Cancer, Vaginal Cancer, and Wilms' Tumor.
Embodiment 225. The isolated antigen-binding protein according to Embodiment 1, wherein the isolated antigen-binding protein includes VH including HCDR1 as set forth in SEQ ID NO: 2, HCDR2 as set forth in SEQ ID NO: 6, HCDR3 as set forth in SEQ ID NO: 15, and VL including LCDR1 as set forth in SEQ ID NO: 42, LCDR2 as set forth in SEQ ID NO: 43, and LCDR3 as set forth in SEQ ID NO: 44.
Embodiment 226. The isolated antigen-binding protein according to Embodiment 225, wherein the isolated antigen-binding protein includes VH as set forth in SEQ ID NO: 70 and VL as set forth in SEQ ID NO: 106.

Without intending to be bound by any theory, the following examples are intended merely to illustrate the fusion proteins, methods of preparation, uses, etc., of the present application and are not intended to limit the scope of the present application.

### EXAMPLES

### Example 1 SJL Mouse Immunization

1.1 Immunogen: Sequence that encoding human TL1A protein (UniprotKB NO. 095150), the DNA sequence of amino acids 72-251 in the extracellular region, was cloned into the mDeZ-Fc vector containing the Fc tag and a recombinant eukaryotic expression plasmid was prepared. The expression plasmid was transiently transfected into Expi293 cells and cultured in larger quantities, and the cell culture supernatant was collected four days later, and the cell culture supernatant was purified to obtain high-purity and biologically active huTL1A-Fc protein, which was frozen at −80° C. for later use. At the same time, cynomolgus monkey TL1A-his protein (UniprotKB NO. G7PRK8) and murine TL1A-his protein (UniprotKB NO. Q5UBV8) was constructed and prepared, and rat TL1A-his protein (Acrobiosystems, TLA-R5249) was purchased for subsequent related experiments. The corresponding protein amino acid sequences are shown in Table 1.

TABLE 1
Amino acid sequences of related proteins

| Human TL1A | MAEDLGLSFGETASVEMLPEHGSCRPKARSSSARWALTCCLVLLPFLAGLTTYLLVSQLRA |
|---|---|
| (M1-L251) | QGEACVQFQALKGQEFAPSHQQVYAPLRADGDKPRAHLTVVRQTPTQHFKNQFPALHWEH |
| | ELGLAFTKNRMNYTNKFLLIPESGDYFIYSQVTFRGMTSECSEIRQAGRPNKPDSITVVITKV |
| | TDSYPEPTQLLMGTKSVCEVGSNWFQPIYLGAMFSLQEGDKLMVNVSDISLVDYTKEDKTF |
| | FGAFLL (SEQ ID 326) |
| Human sTL1A | LKGQEFAPSHQQVYAPLRADGDKPRAHLTVVRQTPTQHFKNQFPALHWEHELGLAFTKNR |
| (L72-L251) | MNYTNKFLLIPESGDYFIYSQVTFRGMTSECSEIRQAGRPNKPDSITVVITKVTDSYPEPTQLL |
| | MGTKSVCEVGSNWFQPIYLGAMFSLQEGDKLMVNVSDISLVDYTKEDKTFFGAFLL (SEQ |
| | ID 327) |
| Cyno sTL1A | LKGQEFAPSHQQVYAPLRADGDKPRAHLTVVRQTPTQHLKNQFPALHWEHELGLAFTKNR |
| (L72-L251) | MNYTNKFLLIPESGDYFVYSQVTFRGMTSECSEIRQAGRPNKPDSITVVITKVTDSYPEPTQL |
| | LMGTKSVCEVGSNWFQPIYLGAMFSLQEGDKLMVNVSDISLVDYTKEDKTFFGAFLL (SEQ |
| | ID 328) |
| Mouse sTL1A | AGQLRVPGKDCMLRAITEERSEPSPQQVYSPPRGKPRAHLTIKKQTPAPHLKNQLSALHWEH |
| (A61-L252) | DLGMAFTKNGMKYINKSLVIPESGDYFIYSQITFRGTTSVCGDISRGRRPNKPDSITVVITKVA |
| | DSYPEPARLLTGSKSVCEISNNWFQSLYLGAMFSLEEGDRLMVNVSDISLVDYTKEDKTFFG |
| | AFLL (SEQ ID 329) |
| Rat sTL1A | TGQLRIPGKDCMFPTVTEERSAPSAQPVYTPSRDKPKAHLTIMRQTPVPHLKNELAALHWEN |
| (T61-I252) | NLGMAFTKNRMNYTNKFLVIPESGDYFIYSQITFRGTTSECGDISRVRRPKKPDSITVVITKV |
| | ADSYPEPAHLLTGTKSVCEISSNWFQPIYLGAMESLEEGDRLMVNVSDISLVDYTKEDKTFF |
| | GAFLI (SEQ ID 330) |
| Human DR3-ECD | QGGTRSPRCDCAGDFHKKIGLFCCRGCPAGHYLKAPCTEPCGNSTCLVCPQDTFLAWENHH |
| (Q25-Q199) | NSECARCQACDEQASQVALENCSAVADTRCGCKPGWFVECQVSQCVSSSPFYCQPCLDCG |
| | ALHRHTRLLCSRRDTDCGTCLPGFYEHGDGCVSCPTSTLGSCPERCAAVCGWRQ (SEQ ID |
| | 331) |

1.2 SJL mouse immunization: 6-8 weeks old SJL mice were immunized with huTL1A-Fc protein. For primary immunization, 50 μg of huTL1A-Fc protein was emulsified with CFA adjuvant and intraperitoneally injected into mice. For subsequent booster immunization, 50 μg of huTL1A-Fc protein was mixed with RIBI adjuvant and intraperitoneally injected into mice. The interval between each immunization is 2 weeks. Serum was isolated after blood collection 7 days after the third immunization, serum antibody titers were detected by ELISA, and mice with high serum titers were selected for subsequent antibody screening.

### Example 2 Antibody Screening

2.1 Screening of anti-TL1A antibody based on Single B sorting memory B cell technology: Biotin conjugation of huTL1A-his protein via a biotin labeling kit (ThermoFisher, A35389) was used to obtain huTL1A-his-Biotin protein. The spleen of mice with qualified immune titer was prepared into a single-cell suspension, and B cells were enriched by magnetic bead sorting, and the huTL1A-his-Biotin protein was incubated with the isolated B cells, and after the incubation, Streptavidin-PE was added, the positive B cells were sorted, and introduced into a 96-well plate to ensure that each well contained only one positive B cell, and sequencing analysis was arranged later.

2.2 Screening of anti-huTL1A murine monoclonal antibody based on phage display technology: total RNA was extracted from mouse spleen single cells using Trizol, RNA was reverse transcribed into cDNA, and then PCR-amplified VH and VL fragments were inserted into the phage vector, and the constructed phage was packaged into a phage library with a capacity of about 3.0E+8 by an auxiliary phage. After three rounds of phage library panning, clones identified as positive by ELISA were selected for sequencing analysis.

### Example 3 Monoclonal Sequencing

The positive B cells sorted by Single B were cloned, RNA was extracted, reverse transcribed into cDNA, amplified, and then sequenced to obtain the VH and VL sequences of anti-huTL1A monoclonal antibody. The clones that were tested positive by Phage ELISA were sequenced to obtain the VH and VL amino acid sequences of the anti-huTL1A monoclonal antibody. The amino acid sequences of VH and VL for all monoclonal antibodies against huTL1A are shown in Table 2:

TABLE 2
Amino acid sequences of VH and VL of anti-TL1A antibodies
screened by Single B and libraries

| Antibody | | | | |
|---|---|---|---|---|
| NQ. | Amino acid sequence (VH) | SEQ ID | Amino acid sequence (VL) | SEQ ID |
| TLA0014 | QIQLVQSGPELKKPGETVKISCKASGYTFT | 342 | DVVMTQTPLSLPVSLGDQASISCRSS | 544 |
| | TYGMNWVKQSPGKGLKWMGWINTYSGV | | QSIVHSNGNTYLEWYLQKPGQSPKLL | |
| | PTYADDFKGRFAFSLETSVNTAYLQINSLK | | IYKVSNRFSGVPDRFSGSGSGTDFTL | |
| | TDDTATYFCAKDAYDNYAMDYWGQGTS | | KISRVEAEDLGVYYCFQGSHVPLTFG | |
| | VTVSS | | AGTKLELK | |
| TLA0015 | QIQLKESGPGLVAPSQSLSITCTVSGFSLSS | 343 | DIQMTQTTSSLSASLGDRVTISCRASQ | 478 |
| | YGVDWVRQSPGKGLEWLGVIWGFGGTNY | | DISNYLNWYQQKPDGTVKLLIYYTSR | |
| | NSALKSRLSISKDNSKSQVFLKMNSLQSDD | | LHSGVPSRFSGSGSGTDYSLTISNLEQ | |
| | SAIYYCASGNFDAMDYWGQGTSVTVSS | | EDIATYFCQQGNTLPFTFGSGTKLEIK | |
| TLA0094 | DVQLQESGPGLVKPSQSLSLTCSVTDYSIT | 344 | DIKMTQSPSSMYASLGERVTITCKAS | 481 |
| | SGYYWNWIRQFPGNKLEWMGYIHFDGSN | | QDINSYLSWFQQKPGKSPKTLIYRAN | |
| | DYNPSLKNRISITRDTSRNQFFLKLNSVTSE | | RLVDGVPSRFSASGSGQDYSLTISSLE | |
| | DTATYYCAREGTSYFDYWGQGTTLTVSS | | YEDMGIYYCLQCDEFPYTFGGGTKL | |
| | | | EIK | |
| TLA0095 | QIQLVQSGPELKKPGETVKISCKASGYTFT | 345 | DIVLTQSPASLPVSLGQRATISCRASK | 479 |
| | TYGMSWVKQAPGKYLKWMGWIDTYSGV | | SVSTSSYSYMHWYQQKPGQPPKLLIK | |
| | PTYADDFKGRFAFSLETSANTAYLQINNLK | | YASNLESGVPARFSGSGSGTDFTLNI | |
| | NEDTATYFCANSFQFAYWGQGTLVTVSA | | HPVEEEDAATYYCQHSKEFPWTFGG | |
| | | | GTKLEIK | |
| TLA0096 | DVQLQESGPGLVKPSQSLSLTCSVTGYSIT | 346 | DIKMTQSPSSMYASLGERVTITCKAS | 480 |
| | SGYYWNWIRQFPGNKLEWMGCISYDGSN | | QDINSFLSWFQQKPGKSPKTLIYRAN | |
| | YYNPSLKNRISFTRDTSKNQFFLKLNSVTT | | RLVDGVPSRFSGSGSGQDYSLTISSLE | |
| | DDTATYYCVREGTSYVDYWGQGTTLTVS | | YEDMGIYYCLQCDEFPYTFGGGTKL | |
| | S | | EIK | |
| TLA0097 | DVQLQESGPGLVKPSQSLSLTCSVTDYSIT | 347 | DIKMTQSPSSMYASLGERVTITCKAS | 481 |
| | SGYYWNWIRQFPGNKLEWMGYIHYDGSN | | QDINSYLSWFQQKPGKSPKTLIYRAN | |
| | DYNPSLKNRISITRDTSRNQFFLKLNSVTSE | | RLVDGVPSRFSASGSGQDYSLTISSLE | |
| | DTATYYCAREGTSYFDYWGQGTTLTVSS | | YEDMGIYYCLQCDEFPYTFGGGTKL | |
| | | | EIK | |
| TLA0098 | QVQLQQSGPELVRPGASVKISCKAPGYTFT | 348 | DIVMTQSHKFMSTSVGDRVSITCKAS | 482 |
| | SHWMQWVRQRPGQGLEWIGEIFPGSGTTY | | QDVSTAVAWYQQKPGQSPKLLIYWA | |
| | YNEKFQGKATLTVDTSSSTAYMQLSSLTS | | SARHTGVPARFTGSGSGTDYTLTISS | |
| | EDSSVHFCARGEGIYYPARSYGPLGYFDV | | VQAEDLALYYCQQHYGTPPTFGGGT | |
| | WGTGTTVTVSS | | KLEIK | |
| TLA0099 | QVQLQQPGAELVMPGASVKLSCKASGYTF | 349 | QIVLTQSPAIMSVSPGEKVTITCSATS | 483 |
| | TNYWMNWVKQRPRQGLEWIGETDPSNTY | | RVTYMHWFQQKPGTAPKLWIYSTSN | |
| | INYNQKFKGKATLTVDKSSNTAYMQLNSL | | VASGVPARFSGSGSVTSYSLTISRME | |
| | TSEDSAVYYCARGGLPYYGMDYWGQGTS | | AADAATYYCQQRSSYPPTFGGGTKL | |
| | VTVSS | | EIK | |
| TLA0100 | QIQLVQSGPELKKPGETVKISCKASGSAFT | 350 | DIMLTQSPASLTVSLGQRATISCRASK | 484 |
| | TYGMSWVKQAPGEGLKWMGYIITYSGVP | | SVSTSSYTYMHWYQQKPGQPPKLLI | |
| | TYADDFKGRFAFSLETSASTAYLQINNLKN | | KYASNLESGVPARFSGSGSGTDFILNI | |
| | EDTATYFCTNFYYFDYWGQGTTLTVSS | | HPVEEEDAATYYCQHSREFPWTFGG | |
| | | | GTKLEIK | |
| TLA0101 | QIQLVQSGPELKKPGETVKISCKASGYTFT | 351 | DIVLTQSPASLAVSLGQRATISCRASK | 485 |
| | TFGMNWVKQAPGKDLKWMGWINTNSGV | | SVSTSSYNYMHWYQQKPGQPPKLLI | |
| | PTYADDFRGRFAFSMETSATTAYLQINNL | | KYASNLESGVPARFSGSGSGTDFTLN | |
| | KNEDTATYFCARGNWEGPGIWFAYWGQG | | IHPVEEEDAATYYCQHSREFPWTFGG | |
| | TLVTVSA | | GTKLEIK | |
| TLA0102 | QVQLQQPGTELVKPGASVKLSCKASGYTF | 352 | QIVLTQSPALMSASPGEKVTMTCSAS | 486 |
| | TSYWMHWVKQRPGQGLEWIGNIDPNNDV | | SSVSYMYYQQKPRSSPKPWIYLTS | |
| | INYNDKFKSKATLTVDKSSSTAYMQLSSLT | | NLASVPARFSGSGSGTSYSLTISSME | |
| | SEDSGVYYCARGIYAGYWGQGTLVTVSA | | AEDAATYYCQQWIIYPPMLTFGAGT | |
| | | | KLELK | |
| TLA0103 | EVQLQQSGAELMKPGASVKLSCKATGYTF | 353 | DIVLTQSPASLAVSLGQRATISCKASQ | 487 |
| | TGYWLEWVKQRPGHGLEWIGETLPGSGY | | SVSFAGISLMHWYQQKPGQQPKLLIY | |
| | NKYNEKFKGKATFTADTSSNTAHMQLSSL | | RASNLEAGVPTRFSGSGSRTDFTLTIN | |
| | TTEDSAIYYCARRGSNYPYWYFDVWGTG | | PVETDDVATYYCQQSNKDPTFGGGT | |
| | TTVTVSS | | KLELK | |
| TLA0104 | QVQLKESGPGLVQPSQSLSITCTVSGFSLTS | 354 | DIVMTQSHKFMSTSVGDRVSITCKAS | 488 |
| | YGVHWVRQSPGKGLEWLGVIWSGGSTDY | | QDVSTAVAWYQQKPGQSPKLLIYSA | |
| | NAAFISRLSISKDNSKSQVFFKMNSLQADD | | SYRYTGVPDRFTGSGSGTDFTFTISSV | |
| | TAIYYCARNDYDYAMDYWGQGTSVTVSS | | QAEDLAVYYCHQHYSTPYTFGGGTK | |
| | | | LEIKR | |
| TLA0105 | QVQLQQSGPELVKPGASVKVSCKASGYTF | 469 | DVVMTQTPLSLPVSLGDQASISCRSS | 489 |
| | TRQDINWVKQRPGQGLEWIGWIYPRDGRT | | QNIVHSNGDTYLDWFLQKPGQSPKL | |
| | EYNEKFKGKVTLTVDTSSSTAYMELHSLT | | LIYKVSNRFSGVPDRFSGSGSGTDFTL | |
| | SEDSAVYFCATYGSSLDNWGQGTTLTVSS | | KITRVESEDLGVYYCFQGSHDPYTFG | |
| | | | GGTKLEIKR | |
| TL10106 | QVQLQQSGPELVKPGASVKLSCKASGYTF | 355 | DVVMTQTPLSLPVSLGDQASISCRSS | 491 |
| | TSIDINWVKQRPGQGLEWIGWIYPRDGRT | | QSIVHSNGDTYLEWYLQKPGQSPKLL | |
| | DYNEKFKGKATLTVDTSSSTAYMELHSLT | | IYKVSNRFSGVPDRFSGSGSGTDFTL | |
| | SEDSAVYFCARYTSSLDYWGQGTTLTVSS | | KISRVEAEDLGVYYCFQGSHVPYTFG | |
| | | | GGTKLEIKR | |
| TLA0107 | EVKLEESGGGLVQPGGSMKLSCVASGFTF | 356 | ENVLTQSPAIMSASPGERVTMTCSAS | 490 |
| | SNYWMNWVRQSPEKGLEWVAQIRLKSDN | | SSVSYMHWYQQKSSTSPKLWISDTS | |
| | YATHYAESVKGRFTISRDDSKSNVYLQMN | | KLASGVPGRFSGSGSGNSYSLTISSME | |
| | NLRAEDTGIYYCTPLLLRSIDYWGQGTTLT | | AEDVATYYCFQESGYPFTFGAGTKLE | |
| | VSS | | LK | |
| TLA0108 | EVQLQQSGPELVKPGASVKLSCKASGYTF | 357 | DVVMTQTPLSLPVSLGDQASISCRSS | 491 |
| | TSIDINWVKQRPGQGLEWIGWIYPRDGRT | | QSIVHSNGDTYLEWYLQKPGQSPKLL | |
| | DYNEKFKGKATLTVDTSSSTAYMELHSLT | | IYKVSNRFSGVPDRFSGSGSGTDFTL | |
| | SEDSAVYFCARYGSSLDYWGQGTTLTVSS | | KISRVEAEDLGVYYCFQGSHVPYTFG | |
| | | | GGTKLEIKR | |
| TLA0109 | QIQLVQSGPELVKPGASVKLSCQASGYTFT | 423 | DVVMTQTPLSLPVSLGDQASISCRSS | 491 |
| | NSDINWVKQRPGQGLEWIGWVYPRDGRT | | QSIVHSNGDTYLEWYLQKPGQSPKLL | |
| | YYNEKFKGKATLTVDTSSSTAYMELYSLT | | IYKVSNRFSGVPDRFSGSGSGTDFTL | |
| | SEDSAVYFCARYAYALNSWGQGTSVTVSS | | KISRVEAEDLGVYYCFQGSHVPYTFG | |
| | | | GGTKLEIKR | |
| TLA0110 | QVQLQQSGPELVKPGASVKVSCKASGYTF | 469 | DVVMTQTPLSLPVSLGDQASISCRSS | 491 |
| | TRQDINWVKQRPGQGLEWIGWIYPRDGRT | | QSIVHSNGDTYLEWYLQKPGQSPKLL | |
| | EYNEKFKGKVTLTVDTSSSTAYMELHSLT | | IYKVSNRFSGVPDRFSGSGSGTDFTL | |
| | SEDSAVYFCATYGSSLDNWGQGTTLTVSS | | KISRVEAEDLGVYYCFQGSHVPYTFG | |
| | | | GGTKLEIKR | |
| TLA0111 | QVQLQQSGPELVKPGASVKLSCKASGYTF | 358 | DVVMTQTPLSLPVSLGDQASISCRSS | 491 |
| | TNQDMNWVKQRPGQGLEWIGWIFPRDGR | | QSIVHSNGDTYLEWYLQKPGQSPKLL | |
| | THHNENFKGKATLTVDTSSSTAYMELHSL | | IYKVSNRFSGVPDRFSGSGSGTDFTL | |
| | TAEDSAVYFCAIYGFAMDQWGQGTSVTV | | KISRVEAEDLGVYYCFQGSHVPYTFG | |
| | SS | | GGTKLEIKR | |
| TLA0112 | QVQLQQPGPELVKPGASVKLSCKASGYTF | 359 | DVVMTQTPLSLPVSLGDQASISCRSS | 491 |
| | TNQDINWVKQRPGQGLEWIGWIFPRDGRT | | QSIVHSNGDTYLEWYLQKPGQSPKLL | |
| | HYNENFKGKATLTVDTSSSTAYMELHSLT | | IYKVSNRFSGVPDRFSGSGSGTDFTL | |
| | AEDSAVYFCAIYGIALDHWGQGTSVTVSS | | KISRVEAEDLGVYYCFQGSHVPYTFG | |
| | | | GGTKLEIKR | |
| TLA0113 | QVQLQQSGPELVKPGASVKLSCKASGYTF | 420 | DVVMTQTPLSLPVSLGDQASISCRSS | 491 |
| | TDYDVNWVKQRPGQGLEWIGWNYPRDG | | QSIVHSNGDTYLEWYLQKPGQSPKLL | |
| | RTNYNEKFKGKATLTVDISSSTAYMELHS | | IYKVSNRFSGVPDRFSGSGSGTDFTL | |
| | LTSEDSAVYFCARYGSALNVWGTGTTVTV | | KISRVEAEDLGVYYCFQGSHVPYTFG | |
| | SS | | GGTKLEIKR | |
| TLA0114 | QVQLQQSGPELVKPGASVKLSCKASGYTF | 447 | DVVMTQTPLSLPVSLGDQASISCRSS | 191 |
| | TNQDINWVKQRPGQGLEWIGWIFPRDGRT | | QSIVHSNGDTYLEWYLQKPGQSPKLL | |
| | HYNENFKGKATLTVDTSSSTAYMELHSLT | | IYKVSNRFSGVPDRFSGSGSGTDFTL | |
| | AEDSAVYFCAIYGFAMDHWGQGTSVTVS | | KISRVEAEDLGVYYCFQGSHVPYTFG | |
| | S | | GGTKLEIKR | |
| TLA0115 | QVQLQQSGPELVKPGASVKLSCRASGYTF | 414 | DVVMTQTPLSLPVSLGDQASISCRSS | 491 |
| | TSYDINWVKQRPGQGLEWIGWNYPRDGR | | QSIVHSNGDTYLEWYLQKPGQSPKLL | |
| | TDYNEKFKGKATLTVDTSSNTAYMELHSL | | IYKVSNRFSGVPDRFSGSGSGTDFTL | |
| | TSEDSAVYFCTRYGSTLDVWGTGTTVTVS | | KISRVEAEDLGVYYCFQGSHVPYTFG | |
| | S | | GGTKLEIKR | |
| TLA0116 | QVQLQQSGPELVKPGTSVKLSCKASGYTF | 445 | DVVMTQTPLSLPVSLGDQASISCRSS | 491 |
| | TNQDINWVKQRPGQGLEWIGWIFPRDGRT | | QSIVHSNGDTYLEWYLQKPGQSPKLL | |
| | HYNENFKGKATLTVDTSSSTAYMELHSLT | | IYKVSNRFSGVPDRFSGSGSGTDFTL | |
| | AEDSAVYFCSIYGIAMDHWGQGTSVTVSS | | KISRVEAEDLGVYYCFQGSHVPYTFG | |
| | | | GGTKLEIKR | |
| TLA0117 | QVQLQQPGPELVKPGASVKLSCKASGYTF | 360 | DVVMTQTPLSLPVSLGDQASISCRSS | 491 |
| | TNQDINWVKQRPGQGLEWIGWIFPRDGRT | | QSIVHSNGDTYLEWYLQKPGQSPKLL | |
| | HYNENFKGKATLTVDTSSSTAYMELHSLT | | IYKVSNRFSGVPDRFSGSGSGTDFTL | |
| | AEDSAVYFCAIYGAAMDHWGQGTSVTVS | | KISRVEAEDLGVYYCFQGSHVPYTFG | |
| | S | | GGTKLEIKR | |
| TLA0118 | EVKLEESGGGLVQPGGSMKLSCVASGFTF | 361 | ENVLTQSPAIMSASPGEKVTMTCSAS | 492 |
| | SNYWMNWVRQSPEKGLEWVAQIRLKSDN | | SSVSYMHWYQQKSSTSPKLWIYDTS | |
| | YATHYAESVKGRFTISRDDSKSSVYLQMN | | KLASGVPGRFSGSGSGNSYSLTISSME | |
| | NLRAEDTGIYYCTPLLLRYFDVWGTGTTV | | AEDVATYYCFQESGYPLTFGAGTKL | |
| | TVSS | | ELK | |
| TLA0119 | EVQLQQSVAELVRPGASVKLSCTASGFNIE | 362 | EILLTQSPAIIVASPGEKVTITCSATSS | 493 |
| | DTYIHWVKQRPEQGLEWIGRIDPAHDKTK | | VIYMNWYQQKPGSSPKIWIYGISNLA | |
| | YAPKFQGKATITADTSSNTAYLQLSSLTSE | | SGVPARFSGSGSGTSFSFTINSMEAED | |
| | DTAIYYCASTGPDWWGQGTTLTVSS | | VATYYCQQRSSYPFTFGSGTKLEIK | |
| TLA0120 | QVQLQQSGAELVRPGTSVKMSCKASGYTF | 363 | DVQITQSPSYLAASPGETITMNCRAS | 494 |
| | TNYWIGWTKQRPGHGLEWIGDIYPGGGYI | | KSIRKYLAWYQEKPGKSNKILIYSGS | |
| | NYNEKFKGKATLTADKSSSTAYMQFSSLT | | TLQSGIPSRFSGSGSGTDFTLTISSLEP | |
| | SEDSAIYYCAREGGYGSGQAWFPYWGQG | | EDFAMYYCQQHNEYPLTFGAGTKLE | |
| | TLVTVSA | | LK | |
| TLA0121 | QVQLQQPGTELVRPGTSVKLSCKASGYTF | 364 | QIVLTQSPAIMSASPGEKVTMTCSASS | 495 |
| | TSYWMHWVKQRPGQGLEWIGVIEPSDSY | | SVSYMHWYQQKSGTSLKRWIYDTSK | |
| | TDYNQKFKGKATLTVDTSSSTAYMQLSSL | | LASGVPARFSGSGSGTSYSLTVSSME | |
| | TSEDSAVYYCARGDYDEVFAYWGQGTLV | | AEDAATYYCQQWTSTPYTFGGGTKL | |
| | TVSA | | EIK | |
| TLA0122 | QVQLQQPGAELVRPGTSVKLSCKASGYTF | 365 | QIVLTQSPAIMSASPGEKVTMTCSASS | 496 |
| | TSYWMHWVKQRPGQGLEWIGVIEPSDSY | | SVSYMHWYQQKSGTSPKRWIYDTSK | |
| | TDYDQKFKGKATLTVDTSSNTAYMQLSSL | | LASGVPARFSGSGSGTSYSLTISTMEA | |
| | TSEDSAVYYCSRGDYDEVFAYWGQGTLV | | EDAATYYCQQWTSTPYTFGGGTKLE | |
| | TVST | | IK | |
| TLA0123 | QVQLQQSGADLVRPGASVTLSCKASGYTF | 366 | DIQMTQTTSSLSASLGDRVTISCRASQ | 497 |
| | SDSEIHWVRQTPVHGLEWIGAIDPETGDIA | | DITNYLNWYQQKPDGTVKLLIYSTSR | |
| | YNQMFKDKATLTADKSSSTAYVELRSLTS | | LHSGVPSRFSGSGSGTDYSLTISNLEQ | |
| | EDSAVYYCTRYGGNSWHFDVWGTGTTVT | | EDIATYFCQQGNTLPLTFGAGTKLEL | |
| | VSS | | K | |
| TLA0124 | EFQLQQSGPELLKPGASVKISCKASGYSFT | 367 | DIVMTQPQKFMPTSVGDRVSVTCKA | 499 |
| | DYNINWVKQRNGKSLEWIGMINPKYGITD | | SQNVGTNVAWYQQKAGQSPKVLIYS | |
| | YNQKFKGKATLTVDQFSSTAYMQVNSLTS | | ASYRYSGVPDRFTGSGSGTAFTLTISN | |
| | DDSAVYYCAREELGHWCFDVWGTGTTVT | | VQSEDLAEYFCHQYNNFPFSFGSGTM | |
| | VSS | | LEIK | |
| TLA0125 | QVQLQQSGAELVRPGTSVKVSCKASGYAF | 368 | DIKMTQSPSSMYASLGERVTITCKAS | 498 |
| | TNYLIEWVNQRPGQGLEWIGVINPGSGGT | | QDINSYLSWFQQKPGKSPKTLIYRAN | |
| | KYNEKFKGKATLTADKSSSTAYMQLSSLT | | RLLDGVPSRFSGSGSGQDYSLTISSLE | |
| | SEDSAVYFCAREGSAGPFDYWGQGTTLTV | | YEDMGIYYCLQYDEFPYTFGGGTKL | |
| | SS | | EIK | |
| TLA0126 | EFQLQQSGPELVKPGASVKISCKASGYSFT | 369 | DIVMTQPQKFMPTSVGDRVSVTCKA | 499 |
| | DYNINWVKQRNGKSLEWIGMINPKYGITD | | SQNVGTNVAWYQQKAGQSPKVLIYS | |
| | YNQKFKGKATLTVDQFSSTAYMQVNSLTS | | ASYRYSGVPDRFTGSGSGTAFTLTISN | |
| | DDSAVYYCAREELGHWCFDVWGTGTTVT | | VQSEDLAEYFCHQYNNFPFSFGSGTM | |
| | VSS | | LEIK | |
| TLA0127 | EVQLQQSGPVLVKPGASVKMSCEASGYTL | 370 | DIKMTQSPSSMYASLGDRVTITCKAS | 500 |
| | TDYYMNWVKQSHGKSLEWIGIINPYNGAS | | QDIDSYLSWFQQKPGKSPKTLMYHT | |
| | NFNQRFRGKASLTFGKSSNTAYMELNSLT | | NRLIVGVPSRFSGTGSGQDYALTISSL | |
| | SEDSAVYYCASDWDVGYWGQGTALTVSS | | EYEDLGIYYCQQYDEFPSTFGSGTKL | |
| | | | EIK | |
| TLA0128 | EVQLVESGGDLVKPGGSLKLSCAASGFTFS | 371 | DIQMTQTTSSLSASLGDRVTISCRASQ | 501 |
| | RYGMSWVRQTPDKRLEWVATISSGGSDT | | DISNYLNWYQQKPDGTVKLLIYYTSR | |
| | YYPDSVKGRFTISRDNAKNTLYLQMSSLK | | LHSGVPSRFSGSGSGTDYSLTISNLDQ | |
| | SEDTAMYYCARHGYDYGHFDYWGQGTT | | EDIATYFCQQGNTLPLTFGAGTKLEL | |
| | LTVSS | | K | |
| TLA0129 | EFQLQQSGPELVKPGASVKISCKASGYSFT | 372 | DNVMTQSQKFMSTSVGDRVSVTCKA | 502 |
| | DYNINWVKQSNGKSLEWIGVINPNYGITD | | SQNVGTNVAWYQQKAGQSPKVLIYS | |
| | YNQKFKGKATLTVDQSSSTAYMQLNSLTS | | ASYRYSGVPDRFTGSGSGTAFTLTISN | |
| | EDSAVYYCAREELGHWCFDVWGTGTTVT | | VQSEDLAEYFCQQYNSFPFTFGSGTK | |
| | VSS | | LEIK | |
| TLA0130 | QVTLKESGPGILQPSQTLNLTCSFSGFSLST | 373 | DVVMTQTPLTLSVTIGQPASISCKSSQ | 503 |
| | FGMGVGWIRQPSGEGLEWLAHIWWDDHE | | SLLDSDGKTYLSWLLQRPGQSPKRLII | |
| | YYNPALKSRLTISKDTSKNQVFLKIANVDT | | LVSKLDSGVPDRFTGSGSGTDFTLKIS | |
| | ADTATYYCNRGFVYWGQGTLVTVSA | | RVEAEDLGVYYCWQGTHFPQTFGGG | |
| | | | TKLEIK | |
| TLA0131 | QVQLQQSGPELVKPGTSVKLSCKASGYTF | 374 | DVLMTQAPLSLPVSLGDQASISCRSS | 504 |
| | TSYDINWMKQRPGQGLEWIGWIFPRDGIT | | QSIVHSNGDTYLDWYLQKPGQSLKL | |
| | KYNEKFKDKATLTVDTSSSAAYMELHSLT | | LIYKLSNRFSGVPDRFSGSGSGTDFTL | |
| | SEDSAVYFCARYGPAMDYWGQGTSVTVS | | KISRVEAEDLGVYHCFQGSHVPYTFG | |
| | S | | GGTKLEIK | |
| TLA0132 | EVQLQQSGPELVKPGASVKLSCKASGYTF | 441 | DVVMTQTPLSLPVSLGDQASISCRSS | 505 |
| | TNQDINWVKQRPGQGLEWIGWIFPRDGRT | | QSIVHGNGNTYLEWYLQKPGQSPKL | |
| | HYNENFKGKATLTVDTSSSTAYMELHSLT | | LIYKVSNRFSGVPDRFSGSGSGTDFTL | |
| | AEDSAVYFCAIYGIALDHWGQGTSVTVSS | | KISRVEAEDLGVYYCFQGSHVPYTFG | |
| | | | GGTKLELKR | |
| TLA0133 | QIQLVQSGPELVKPGASVKVSCKASGYTFT | 375 | DVVMTQTPLSLPVSLGDQASISCRSS | 506 |
| | RQDINWVKQRPGQGLEWIGWIYPRDGRTE | | QSIVHSNGNTYLEWYLQKPGQSPKLL | |
| | YNEKFKGKVTLTVDTSSSTAYMELHSLTS | | IYKVSNRFSGVPDRFSGSGSGTDFTL | |
| | EDSAVYFCATYGSSLDNWGQGTTLTVSS | | KISRVEAEDLGVYYCFQGSHVPYTFG | |
| | | | GGTKLELKR | |
| TLA0134 | EVQLQQSGPELVKPGASVKVSCKASGYTF | 376 | DVVMTQTPLSLPVSLGDQASISCRSS | 506 |
| | TRQDINWVKQRPGQGLEWIGWIYPRDGST | | QSIVHSNGNTYLEWYLQKPGQSPKLL | |
| | KYNEKFKGKVTLTVDTSSSTAYMELHSLT | | IYKVSNRFSGVPDRFSGSGSGTDFTL | |
| | SEDSAVYFCATYGSSLDNWGQGTTLTVSS | | KISRVEAEDLGVYYCFQGSHVPYTFG | |
| | | | GGTKLELKR | |
| TLA0135 | EVQLQQSGPELVKPGASVKLSCKASGYTF | 377 | DVVMTQTPLSLPVSLGDQASISCRSS | 507 |
| | TNQDINWVKQRPGQGLEWIGWIFPRDGRT | | QSIVHSNGSTYLEWYLQKPGQSPKLL | |
| | HYNENFKGKATLTVDTSSSTAYMELHSLT | | IYKVSNRFSGVPDRFSGSGSGTDFTL | |
| | AEDSAVYFCAIYGIAMDHWGQGTSVTVSS | | KISRVEAEDLGVYYCFQGSHVPYTFG | |
| | | | GGTKLEIK | |
| TLA0136 | QIQLVQSGPELVKPGASVKLSCKASGYTFT | 378 | DVVMTQTPLSLPVSLGDQASISCRSS | 547 |
| | NYDINWVKQRPGQGLEWIGWIYPRDGSTY | | QSIVHSNGNTYLEWYLQKPGQSPKLL | |
| | YNEKFKGKATLTVDTSSTTAYMEFHSLTS | | IYKVSNRFSGVPDRFSGSGSGTDFTL | |
| | EDSAVYFCARYAYALNSWGQGTSVTVSS | | KISRVEAEDLGVYYCFQGSHVPYTFG | |
| | | | GGTKLEIKR | |
| TLA0137 | QVQLKQSGPELVRPGASVKLSCKASGYSF | 379 | DVVMTQTPLSLPVSLGDQASISCRSS | 508 |
| | TSGDINWVKQRPGQGLEWIAWIYPRDGST | | QNIVHSNGDTYLDWFLQKPGQSPQL | |
| | EYNEKFKGKATLTVDTSSSTAYMELHSLT | | LIYKVSNRFSGVPDRFSGSGSGTDFTL | |
| | SEDSAVYFCARYDCAFVYWGQGTLVTVS | | KISRVEAEDLGIYYCFQGSHVPYTFG | |
| | A | | GGTKLEIK | |
| TLA0138 | QVQLQQSGPELVKPGASVKLSCKASGYTF | 380 | DVVMTQTPLSLPVSLGDQASISCRSS | 553 |
| | TNQDINWVKQRPGQGLEWIGWIFPRDGRT | | QSIVHSNGNTYLEWYLQKPGQSPKLL | |
| | HYNENFKGKATLTVDTSSSTAFMELHSLT | | IYKVSNRFSGVPDRFSGSGSGTDFTL | |
| | AEDSAVYFCAIYGIALDHWGQGTSVTVSS | | KISRVEAEDLGVYYCFQGSHVPYTFG | |
| | | | GGTKLEIK | |
| TLA0139 | QVQLQQSGPELVKPGASVKLSCKASGYTF | 381 | DVVMTQTPLSLPVSLGDQASISCRSS | 550 |
| | TDYDVNWVKQRPGQGLEWIGWNYPRDG | | QSIVHSNGDTYLDWFLQKPGQSPKLL | |
| | RTNYNEKFKGKATLTVDISSSTAYMELHS | | IYKVSNRFSGVPDRFSGSGSGTDFTL | |
| | LTSEDSAVYFCARYGSALNVWGTGTSVTV | | KISRVEAEDLGVYYCFQGSHVPYTFG | |
| | SS | | GGTKLEIK | |
| TLA0140 | QIQLVQSGPELVKPGASVKLSCKASGYTFT | 382 | DVVMTQTPLSLPVSLGDQASISCRSS | 538 |
| | KYDINWVKQRPGQGLEWIGWIYPRDGSTK | | QNIVHSNGDTYLDWFLQKPGQSPKL | |
| | YNEKFKGKATLTVDTSSNTAYMELHSLTS | | LIYKVSNRFSGVPDRFSGSGSGTDFTL | |
| | EDSAVYFCARYSAAMDYWGQGTSVTVSS | | KISRVEAEDLGVYYCFQGSHVPYTFG | |
| | | | GGTKLEIK | |
| TLA0141 | QVQLQQPGAELGKPGASVKLSCKASGYTF | 383 | DVVMTQTPLSLPVSLGDQASISCRSS | 553 |
| | TNYDINWVKQRPGQGLEWIGWNYPRDGR | | QSIVHSNGNTYLEWYLQKPGQSPKLL | |
| | TKYNEKFKGKATLTVDTSSSTAYMELHSL | | IYKVSNRFSGVPDRFSGSGSGTDFTL | |
| | TSEDSAVYFCARYGYAMDYWGQGTSVTV | | KISRVEAEDLGVYYCFQGSHVPYTFG | |
| | SS | | GGTKLEIK | |
| TLA0142 | QVQLQQPGAELGKPGASVKLSCKASGYTF | 384 | DVVMTQTPLSLPVSLGDQASISCRSS | 509 |
| | TNYDINWMKLRPGQGLEWIGWNYPRDGR | | QSIVHSNGHTYLEWYLQKPGQSPKLL | |
| | TKYNEKFKGKATFTVDTSSSTAYMELHSL | | IYKVSNRFSGVPDRFSGSGSGTDFTL | |
| | TSEDSAVYFCARYGYAMDYWGQGTSVTV | | KISRVEAEDLGVYYCFQGSHVPYTFG | |
| | SS | | GGTKLEIK | |
| TLA0143 | QIQLVQSGPELVKPGASVKLSCRASGYTFT | 385 | DVVMTQTPLSLPVSLGDQASISCRPS | 510 |
| | SYDINWVKQRPGQGLEWIGWIFPRDGRTE | | QTIVHSNGDTYLDWFLQKPGQSPKLL | |
| | YNAKFKGKATLTVDTSSNTAYMELHSLTS | | IYKVSNRFSGVPDRFSGSGSGTDFTL | |
| | DDSAVYFCARYGSTLDVLGAGTTVTVSS | | KISRVEAEDLGVYYCFQGSHVPYTFG | |
| | | | GGTRLEIK | |
| TLA0144 | EVQLQQSGPELVKPGASVKLSCKASGYTF | 386 | DVVMTQTPLSLPVSLGDQASISCRSS | 550 |
| | TNQDINWVKQRPGQGLEWIGWIFPRDGRT | | QSIVHSNGDTYLDWFLQKPGQSPKLL | |
| | HYNENFKDKATLTVDTSSSTAYMEVHSLT | | IYKVSNRFSGVPDRFSGSGSGTDFTL | |
| | AEDSAVYFCAIYGIALDHWGQGTSVTVSS | | KISRVEAEDLGVYYCFQGSHVPYTFG | |
| | | | GGTKLEIK | |
| TLA0145 | QVQLQQSGPELVKPGASVKVSCKASGYTF | 469 | DVVMTQTPLSLPVSLGDQASISCRSS | 553 |
| | TRQDINWVKQRPGQGLEWIGWIYPRDGRT | | QSIVHSNGNTYLEWYLQKPGQSPKLL | |
| | EYNEKFKGKVTLTVDTSSSTAYMELHSLT | | IYKVSNRFSGVPDRFSGSGSGTDFTL | |
| | SEDSAVYFCATYGSSLDNWGQGTTLTVSS | | KISRVEAEDLGVYYCFQGSHVPYTFG | |
| | | | GGTKLEIK | |
| TLA0146 | QIQLVQSGPELVKPGASVKLSCKASGYTFT | 387 | DVVMTQTPLSLPVSLADQASISCRSS | 552 |
| | RQDINWVKQRPGQGLEWIGWIYPRDGSTK | | QSIVHDNGDTYLEWYLQKPGQSPRL | |
| | YNEKFKGKVTLTVDTSSSTAYMELHSLTS | | LIYKVSNRFSGVPDRFSGSGSGTDFTL | |
| | EDSAVYFCATYGSSLDNWGQGTTLTVSS | | KINRVEAEDLGVYYCFQGSHVPYTF | |
| | | | GGGTKLEIK | |
| TLA0147 | QVQLQQSGPELVKPGASVKIPCKTSGYTFT | 388 | DVVMTQTPLSLPVSLGDQASISCRSS | 511 |
| | DYNINWVKRSHGKSLEWIGDINPDSGGTS | | QSIVHSNGNTYLEWYLQKPGQSPKLL | |
| | YNQRFKDKATLTVDKSSSTAYMELRSLTS | | IYKVSNRFSGVPDRFSGSGSGTDFTL | |
| | GDTAVYYCARWGYYGGRGFDYWGQGTT | | KISRVEAEDLGVYYCFQGSHVPLTFG | |
| | LTVSS | | DGTKLEIK | |
| TLA0148 | QVQLQQSGAELGKPGASVKLSCKASGYTF | 389 | DVVMTQTPLSLPVSLGDQASISCRSS | 553 |
| | TNYDINWMKQRPGQGLEWIGWNYPRDGR | | QSIVHSNGNTYLEWYLQKPGQSPKLL | |
| | TKYNEKFKGKATLTVDTSSSTAYMELHSL | | IYKVSNRFSGVPDRFSGSGSGTDFTL | |
| | TSEDSAVYFCARYGYAMDYWGQGTSVTV | | KISRVEAEDLGVYYCFQGSHVPYTFG | |
| | SS | | GGTKLEIK | |
| TLA0149 | EVQLQQSGPELVKPGASVKVSCKASGYTF | 471 | DVVMTQTPLSLPVSLGDQASISCRSS | 553 |
| | TRQDINWVKQRPGQGLEWIGWIYPRDGRT | | QSIVHSNGNTYLEWYLQKPGQSPKLL | |
| | EYNEKFKGKVTLTVDTSSSTAYMELHSLT | | IYKVSNRFSGVPDRFSGSGSGTDFTL | |
| | SEDSAVYFCATYGSSLDNWGQGTTLTVSS | | KISRVEAEDLGVYYCFQGSHVPYTFG | |
| | | | GGTKLEIK | |
| TLA0150 | EVQLQQSGPDLVKPGASVKLSCKASGYTF | 390 | DVVMTQTPLSLPVSLGDQASISCRSS | 512 |
| | ASYDINWVKQRPGQGLEWIGWIYPRDGRT | | QSIVHSNGDTYLDWFLQKPGQSPKLL | |
| | DYNEKFKGKATLTVDTSSSTAYMELHSLT | | IYKVSNRFSGVPDRFSGSGSGTDFTL | |
| | SEDSAVYFCARYGYALGYWGQGTSVTVS | | KITRVESEDLGVYYCFQGSHDPYTFG | |
| | S | | GGTKLEIK | |
| TLA0151 | QVQLQQSGPELAKPGASVKLSCKASGYTF | 391 | DVVMTQTPLSLPVSLGDQASISCRSS | 513 |
| | TSRDINWVKQRPGQGLEWIGWIYPRDGRT | | QSVVHNNGNTYLEWYLQKPGQSPKL | |
| | NYNEKFKDKATLTVDTSSSTAYMDLHSLT | | LIYKVSNRFSGVPDRFSGSGSGTDFTL | |
| | SEDSAVYFCATYGSSLDHWGQGTTLTVSS | | KISRVEAEDLGVYYCFQGSHVPYTFG | |
| | | | GGTKLELK | |
| TLA0152 | EVQLQQSGPELVKPGASVKLSCKASGYTF | 392 | DVVMTQTPLSLPVSLGDQASISCRSS | 553 |
| | TNQDINWVKQRPGQGLEWIGWIFPRDGRT | | QSIVHSNGNTYLEWYLQKPGQSPKLL | |
| | HYNENFKGKATLTVDTSSSTAYMELHSLT | | IYKVSNRFSGVPDRFSGSGSGTDFTL | |
| | AEDSAVYFCAIYGAAMDHWGQGTSVTVS | | KISRVEAEDLGVYYCFQGSHVPYTFG | |
| | S | | GGTKLEIK | |
| TLA0153 | QVQLQQSGPELVKPGTSVKLSCKASGYTF | 393 | DVVMTQTPLSLPVSLGDQASISCRSS | 553 |
| | TDQDINWVKQRPGQGLEWIGWIFPRDGRT | | QSIVHSNGNTYLEWYLQKPGQSPKLL | |
| | HYNEKFKGKATLTVDTSSSTAYMELHSLT | | IYKVSNRFSGVPDRFSGSGSGTDFTL | |
| | SEDSAVYFCSIYGSAMDLWGQGTSVTVSS | | KISRVEAEDLGVYYCFQGSHVPYTFG | |
| | | | GGTKLEIK | |
| TLA0154 | QVQLQQSGPELVKPGASVKLSCKASGYTF | 394 | DVVMTQTPLSLPVSLGNQASISCRSS | 528 |
| | TNQDINWVKQRPGQGLEWIGWIFPRDGRT | | QDIVHTNGDTYLDWYLQKPGQSPKL | |
| | HNNENFKGKATLTVDTSSSTAYMELHSLT | | LIYKVSNRFSGVPDRFSGSGSGTDFTL | |
| | AEDSAVYFCAIYGFAMDHWGQGTSVTVS | | KINRVEAEDLGVYYCFQGSHVPYTF | |
| | S | | GGGTKLELK | |
| TLA0155 | QVQLQQSGPELVKPGASVKLSCKASGYTF | 443 | DVVMTQTPLSLPVSLGDQASISCRSS | 553 |
| | TNQDINWVKQRPGQGLEWIGWIFPRDGRT | | QSIVHSNGNTYLEWYLQKPGQSPKLL | |
| | HYNENFKGKATLTVDTSSSTAYMELHSLT | | IYKVSNRFSGVPDRFSGSGSGTDFTL | |
| | AEDSAVYFCAIYGIALDHWGQGTSVTVSS | | KISRVEAEDLGVYYCFQGSHVPYTFG | |
| | | | GGTKLEIK | |
| TLA0156 | EVQLQQSGPELVKPGASVKLSCKASGYTS | 395 | DVVMTQTPLSLPVSLGDQASISCRSS | 514 |
| | TSYDINWVKQRPGQGLEWIGWIYPRDGRT | | QSIVHSNGDTYLEWYLQKPGQSPKLL | |
| | NYNEKFKGKATLTVDTSSSTAFMELHSLT | | IYKVSNRFSGVPDRFSGSGSGTDFTL | |
| | SEDSAVYFCARYSAAMDYWGQGTSVTVS | | KISRVEAEDLGVYYCFQGSHVPYTFG | |
| | S | | GGTKLEIK | |
| TLA0157 | QVQLQQPGSELVRPGASVKIPCKTSGYSFT | 396 | DVVMTQTPLSLPVSLGDQASISCRSS | 544 |
| | DFNINWVRQSHGKSLEWIGDINPDSGGTIY | | QSIVHSNGNTYLEWYLQKPGQSPKLL | |
| | NQKFKGKATLTVDKSSSTAYMELRSLTSE | | IYKVSNRFSGVPDRFSGSGSGTDFTL | |
| | DTAVYYCARWGYYGGEGFDYWGQGTTL | | KISRVEAEDLGVYYCFQGSHVPLTFG | |
| | TVSS | | AGTKLELK | |
| TLA0158 | QVQLQQSGPELVKPGASVKLSCKASGYTF | 442 | DVVMTQTPLSLPVSLGDQASISCRSS | 531 |
| | TNQDINWVKQRPGQGLEWIGWIFPRDGRT | | QTIVHSNGDTYLDWYLQKPGQSPKL | |
| | HYNENFKGKATLTVDTSSNTAYMELHSLT | | LISKVSNRFSGVPDRFSGSGSGTDFTL | |
| | AEDSAVYFCAIYGIALDHWGQGTSVTVSS | | KISSVEAEDLGVYYCFQGSHVPYTFG | |
| | | | GGTKLEIK | |
| TLA0159 | QIQLVQSGPELVKPGASVKLSCKASGYTFT | 397 | DVVMTQTPLSLPVSLGDQASISCRSS | 557 |
| | SSDINWVKQRPGQGPEWIGWIYPRDGRTH | | QSIVHSNGNTYLEWYLQKPGQSPKLL | |
| | YNEKFKGKATLTVDTSSSTAYMELHSLTS | | IYKVSNRFSGVPDRFSGSGSGTDFTL | |
| | EDSAVYFCARYAYALNSWGQGTSVTVSS | | KISRVEAEDLGVYYCFQGSHVPYTFG | |
| | | | SGTKLEIK | |
| TLA0160 | QVQLQQPGPELVKPGASVRVSCKASGYTF | 398 | DVVMTQTPLSLPVSLGDQASISCRSS | 514 |
| | TRQDINWVKQRAGQGLEWIGWIYPRDGR | | QSIVHSNGDTYLEWYLQKPGQSPKLL | |
| | TEYNEKFKGKVTLTVDTSSSTAYMELHSL | | IYKVSNRFSGVPDRFSGSGSGTDFTL | |
| | TSEDSAVYFCATYGSSLDNWGQGATLTVS | | KISRVEAEDLGVYYCFQGSHVPYTFG | |
| | S | | GGTKLEIK | |
| TLA0161 | QVQLQQSGPELVKPGASVKLSCKASGYTF | 399 | DVVMTQTPLSLPVSLGDQASISCRSS | 515 |
| | TSYDINWVKQRPGQGLEWIGWIFPRDGDT | | QSIVHTNGDTYLDWYLQKPGQSPKL | |
| | KYNEKFKGKATLTVDTSSSTAFMELHSLT | | LIYKVSNRFSGVPDRFSGSGSGTDFTL | |
| | SEDSAVYFCARYGSTLDVWGTGTTVTVSS | | KISRVEAEDLGVYYCFQGSHVPYTFG | |
| | | | GGTKLEIK | |
| TLA0162 | QIQLVQSGPELVMPGASVKLSCQASGYTF | 400 | DVVMTQTPLSLPVSLGDQASISCRSS | 553 |
| | TNSDINWVKQRPGQGLEWIGWVYPGDGS | | QSIVHSNGNTYLEWYLQKPGQSPKLL | |
| | TYYNEKFKGKATLTVDTSSSTAYMDLYSL | | IYKVSNRFSGVPDRFSGSGSGTDFTL | |
| | TSEDSAVYFCARYAYALNSWGQGTSVTVS | | KISRVEAEDLGVYYCFQGSHVPYTFG | |
| | S | | GGTKLEIK | |
| TLA0163 | QIQLVQSGPELVKPGASVKLSCKASGYTFT | 401 | DVVMTQTPLSLPVSLGDQASISCRSS | 516 |
| | NSDINWVKQRPGQGLEWIGWIYPRDGSTY | | QSIVHDNGDTYLEWYLQKPGQSPKL | |
| | YNEKFKGKATLTVDTSSTTAYMEFHSLTS | | LIYKVSNRFSGVPDRFSGSGSGTDFTL | |
| | EDSAVYFCARYAYALNSWGQGTSVTVSS | | KINRVEAEDLGVYYCFQGSHVPYTF | |
| | | | GGGTKLEIK | |
| TLA0164 | QVQLQQSGPEVVKPGASVKLSCKASGYTF | 402 | DVVMTQTPLSLPVSLGDQASISCRSS | 553 |
| | TSYDINWVKQRPGQGLEWIGWIFPRDGRT | | QSIVHSNGNTYLEWYLQKPGQSPKLL | |
| | EYNEKFKDKATLTVDTSSSTAYMELHSLT | | IYKVSNRFSGVPDRFSGSGSGTDFTL | |
| | SEDSAFYFCARYGSSLDYWGQGTTLTVSS | | KISRVEAEDLGVYYCFQGSHVPYTFG | |
| | | | GGTKLEIK | |
| TLA0165 | QIQLVQSGPELVKPGASVKLSCQASGYTFT | 403 | DVVMTQTPLSLPVSLGDQASISCRSS | 517 |
| | NSDINWVKQRPGQGLDWIGWVYPRDGST | | QNIVHSNGNTYLEWYLQKPGQSPKL | |
| | YYNEKFKGKATLTVDTSSSTAYMELYSLT | | LIYKVSNRFSGVPDRFSGSGSGTDFTL | |
| | SEDSAVYFCARYAYALNYWGQGTSVTVS | | KISRVEAEDLGVYYCFQGSHVPYTFG | |
| | S | | GGTKLEIK | |
| TLA0166 | QIQLVQSGPELVKPGASVKLSCKASGYTFT | 404 | DVVMTQTPLSLPVSLGDQASISCRSS | 553 |
| | SIDINWVKQRPGQGLEWIGWIYPRDGRTD | | QSIVHSNGNTYLEWYLQKPGQSPKLL | |
| | YNEKFKGKATLTVDTSSSTAYMELHSLTS | | IYKVSNRFSGVPDRFSGSGSGTDFTL | |
| | EDSAVYFCARYTSSLDYWGQGTTLTVSS | | KISRVEAEDLGVYYCFQGSHVPYTFG | |
| | | | GGTKLEIK | |
| TLA0167 | QVQLQQSGPELVKPGASVKLSCKASGYTF | 405 | DVVMTQTPLSLPVSLGDQASISCRSS | 553 |
| | TSIDINWVKQRPGQGLEWIGWIYPRDGRT | | QSIVHSNGNTYLEWYLQKPGQSPKLL | |
| | NYNEKFKGKATLTVDTSSSTAYMDLHSLT | | IYKVSNRFSGVPDRFSGSGSGTDFTL | |
| | SEDSAVYFCARYTSSLDYWGQGTTLTVSS | | KISRVEAEDLGVYYCFQGSHVPYTFG | |
| | | | GGTKLEIK | |
| TLA0168 | QVQLQQSGPELVKPGASVKLSCRASGYTF | 406 | DVVMTQTPLSLPVSLGDQASISCRSS | 550 |
| | TSYDINWVKQRPGQGLEWIGWNYPRDGR | | QSIVHSNGDTYLDWFLQKPGQSPKLL | |
| | TDYNEKFKGKATLTVDTSSNTAYMDLHSL | | IYKVSNRFSGVPDRFSGSGSGTDFTL | |
| | TSEDSAVYFCTRYGSTLDVWGTGTSVTVS | | KISRVEAEDLGVYYCFQGSHVPYTFG | |
| | S | | GGTKLEIK | |
| TLA0169 | QIQLVQSGPELVKPGASVKLSCQASGYTFT | 407 | DVVMTQTPLSLPVSLGDQASISCRSS | 558 |
| | NSDINWVKQRPGQGLEWIGWVYPRDGSA | | QSIVHSNGNTYLEWYLQKPGQSPKLL | |
| | YYNEKFKGKATLTVDTSSSTAYMELYSLT | | IYKVSNRFSGVPDRFSGSGSGTDFTL | |
| | SEDSAVYFCARYAYALNFWGQGTSVTVSS | | KISRVEAEDLGVYYCFQGSHVPYTFG | |
| | | | GGTKLELK | |
| TLA0170 | QVQLKQSGPELVKPGASVKVSCKASGYTF | 408 | DVVMTQTPLSLPVSLADQASISCRSS | 552 |
| | TRQDINWVKQRPGQGLEWIGWIYPRDGRT | | QSIVHDNGDTYLEWYLQKPGQSPRL | |
| | EYNEKFKGKVTLTVDTSSSTAYMELHSLT | | LIYKVSNRFSGVPDRFSGSGSGTDFTL | |
| | SEDSAVYFCATYGSSLDNWGQGTTLTVSS | | KINRVEAEDLGVYYCFQGSHVPYTF | |
| | | | GGGTKLEIK | |
| TLA0171 | QVQLQQPGPELVKPGASVKLSCKASGYTF | 409 | DVVMTQTPLSLPVSLGDQASISCRSS | 553 |
| | TNQDINWVKQRPGQGLEWIGWIFPRDGRT | | QSIVHSNGNTYLEWYLQKPGQSPKLL | |
| | HYNEKFKGKATLTVDTSSSTAYMELHSLT | | IYKVSNRFSGVPDRFSGSGSGTDFTL | |
| | AEDSAVYFCAIYGSAMDQWGQGTSVTVS | | KISRVEAEDLGVYYCFQGSHVPYTFG | |
| | S | | GGTKLEIK | |
| TLA0172 | EVQLQQSGPELVKPGASVKLSCKASGYTF | 410 | DVVMTQTPLSLPVSLGDQASISCRSS | 553 |
| | TNSDINWVKQRPGQGLEWIGWIYPRDGST | | QSIVHSNGNTYLEWYLQKPGQSPKLL | |
| | YYNEKFKGKATLTVDTSSTTAYMEFHSLT | | IYKVSNRFSGVPDRFSGSGSGTDFTL | |
| | SEDSAVYFCARYAYALNSWGQGTSVTVSS | | KISRVEAEDLGVYYCFQGSHVPYTFG | |
| | | | GGTKLEIK | |
| TLA0173 | QVQLQQSGAELMKPGASVKLSCKATGYT | 412 | DIVLTQSPASLAVSLGQRATISCRASE | 518 |
| | FTGYWLEWVKQRPGHGLEWIGETLPGSG | | SVDDYGISFMHWYQQKPGQPPKLLI | |
| | YNKYNEKFKGKATFTADTSSNTAHMQLSS | | YRASNLESGIPARFSGSGSRTDFTLTI | |
| | LTTEDSAIYYCARRGSNYPYWYFDVWGT | | DPVETDDVATYYCQQSYEDPPTFGA | |
| | GTTVTVSS | | GTKLEIK | |
| TLA0174 | QVQLQQPGAELMKPGASVKLSCKATGYT | 411 | DIVLTQSPASLAVSLGQRATISCRASE | 519 |
| | FTAYWLEWVKQRPGHGLEWIGEILPGSGY | | SVDNYGISFMHWYQQKPGQPPKLLV | |
| | AYYNAKFKGKATFTADISSNTAYMQLSGL | | YRASNLESGIPARFSGSGSRTDFTLTI | |
| | TTEDSAIYYCARRGSNYPYWYFDVWGTG | | NPVETDDVATYYCQQSNKDPPTFGG | |
| | TTVTVSS | | GTKLEIK | |
| TLA0175 | QVQLQQSGAELMKPGASVKLSCKATGYT | | DIVLTQSPASLAVSLGQRATISCRASE | 520 |
| | FTGYWLEWVKQRPGHGLEWIGETLPGSG | 412 | SVDNYGISFMHWYQQKPGQPPKLLI | |
| | YNKYNEKFKGKATFTADTSSNTAHMQLSS | | YRASNLESGIPARFSGSGSRTDFTLTI | |
| | LTTEDSAIYYCARRGSNYPYWYFDVWGT | | NPVETDDVATYYCQQSNKDPPTFGA | |
| | GTTVTVSS | | GTKLELK | |
| TLA0176 | QVQLQQSGAELMKPGASVKLSCKATGYT | 413 | DIVLTQSPASLAVSLGQRATISCRASK | 521 |
| | FTGYWLEWVKQRPGHGLEWIGETLPGSG | | SVSTSSYSYMHWYQQKPGQPPKLLIK | |
| | YNKYNEKFKGKATFTADTSSNTAHMQLSS | | YASNLESGVPARFSGSGSGTDFTLNI | |
| | LTTEDSAIYYCARRGSNYPYWYFDVWGT | | HPVEEEDAATYYCQHSREFPTFGGGT | |
| | GTSVTVSS | | KLEIK | |
| TLA0177 | QVQLQQSGPELVKPGASVKLSCRASGYTF | 414 | DVVMTQTPLSLPVSLGDQASISCRSS | 538 |
| | TSYDINWVKQRPGQGLEWIGWNYPRDGR | | QNIVHSNGDTYLDWFLQKPGQSPKL | |
| | TDYNEKFKGKATLTVDTSSNTAYMELHSL | | LIYKVSNRFSGVPDRFSGSGSGTDFTL | |
| | TSEDSAVYFCTRYGSTLDVWGTGTTVTVS | | KISRVEAEDLGVYYCFQGSHVPYTFG | |
| | S | | GGTKLEIK | |
| TLA0178 | QIQLVQSGPELVKPGASVKLSCRASGYTFT | 415 | DVVMTQTPLSLPVSLGDQASISCRSS | 522 |
| | SYDINWVKQRPGQGLEWIGWNYPRDGRT | | QSIVHSNGDTYLDWYLEKPGQSPKLL | |
| | DYNEKFKGKATLTVDTSSNTAYMELHSLT | | IYKVSNRFSGVPDRFSGSGSGTDFTL | |
| | SEDSAVYFCTRYGSTLDVWGTGTTVTVSS | | KISRVEAEDLGVYYCFQGSHVPYTFG | |
| | | | GGTKLEIKR | |
| TLA0179 | QVQLQQPGPELVKPGASVKLSCRASGYTF | 416 | DVVMTQTPLSLPVSLGDQASISCRSS | 533 |
| | TSYDINWVKQRPGQGLEWIGWIFPRDGRT | | QSIVHNNGDTYLDWYLEKPGQSPKL | |
| | EYNAKFKGKATLTVDTSSNTAYMELHSLT | | LIYKVSNRFSGVPDRFSGSGSGTDFTL | |
| | SDDSAVYFCARYGSTLDVLGAGTTVTVSS | | KISRVEAEDLGVYYCFQGSHVPYTFG | |
| | | | GGTKLEIK | |
| TLA0180 | EVQLQQSGPELVKPGASVKLSCRASGYTF | 417 | DVVMTQTPLSLPVSLGDQASISCRSS | 529 |
| | TSYDINWVKQRPGQGLEWIGWIFPRDGRT | | QSIVHSNGDTYLDWFLQKPGQSPKLL | |
| | EYNEKFKGKATLTVDTSSNTAYMELHSLT | | IYKVSNRFSGVPDRFSGSGSGTDFTL | |
| | SEDSAVYFCARYGSTLDVWGTGTTVTVSS | | KISRVEAEDLGVYYCFQGSHVPYTFG | |
| | | | GGTKLELK | |
| TLA0181 | QIQLVQSGPELVKPGASVKLSCRASGYTFT | 418 | DVVMTQTPLSLPVSLGDQASISCRSS | 523 |
| | SYDINWVKQRPGQGLEWIGWIFPRDGRTE | | QSIVHNNGDTYLDWYLQKPGQSPKL | |
| | YNEKFKGKVTLTVDTSSNTAYMELHSLTS | | LIYKVSNRFSGVPDRFSGSGSGTDFTL | |
| | EDSAVYFCARYGSTLDVWGPGTTVTVSS | | KISRVEAEDLGVYYCFQGSHVPYTFG | |
| | | | GGTKLELK | |
| TLA0182 | QVQLQQFGPELVKPGSSVKLSCKASGYTF | 419 | DVVMTQTPLSLPVSLGDQASISCRSS | 558 |
| | TDYDVNWVKQRPGQGLEWIGWNYPRDG | | QSIVHSNGNTYLEWYLQKPGQSPKLL | |
| | RTNYNEKFKGKATLTVDTSSSTAYMELHS | | IYKVSNRFSGVPDRFSGSGSGTDFTL | |
| | LTSEDSAVYFCARYGSALNVWGTGTTVTV | | KISRVEAEDLGVYYCFQGSHVPYTFG | |
| | SS | | GGTKLELK | |
| TLA0183 | QVQLQQSGPELVKPGASVKLSCKASGYTF | 420 | DVVMTQTPLSLPVSLGDQASISCRSS | 524 |
| | TDYDVNWVKQRPGQGLEWIGWNYPRDG | | QSIVHGNGNTYLEWYLQKPGQSPKL | |
| | RTNYNEKFKGKATLTVDISSSTAYMELHS | | LIYKVSNRFSGVPDRFSGSGSGTDFTL | |
| | LTSEDSAVYFCARYGSALNVWGTGTTVTV | | KISRVEAEDLGVYYCFQGSHVPYTFG | |
| | SS | | GGTKLEIK | |
| TLA0184 | QVQLQQSGPELVKPGASVKLSCKASGYTF | 420 | DVVMTQTPLSLPVSLGDQASISCRSS | 531 |
| | TDYDVNWVKQRPGQGLEWIGWNYPRDG | | QTIVHSNGDTYLDWYLQKPGQSPKL | |
| | RTNYNEKFKGKATLTVDISSSTAYMELHS | | LISKVSNRFSGVPDRFSGSGSGTDFTL | |
| | LTSEDSAVYFCARYGSALNVWGTGTTVTV | | KISSVEAEDLGVYYCFQGSHVPYTFG | |
| | SS | | GGTKLEIK | |
| TLA0185 | QIQLVQSGPELVKPGASVKLSCQASGYTFT | 421 | DVVMTQTPLSLPVSLGDQASISCRSS | 553 |
| | NSDINWVKQRPGQGLEWIGWVYPRDGST | | QSIVHSNGNTYLEWYLQKPGQSPKLL | |
| | YYNEKFKGKATLTVDTSSSTAYMELYSLT | | IYKVSNRFSGVPDRFSGSGSGTDFTL | |
| | SEDSAVYFCARYAYALNSWGQGTSVTVSS | | KISRVEAEDLGVYYCFQGSHVPYTFG | |
| | | | GGTKLEIK | |
| TLA0186 | QIQLVQSGPELVKPGASVKLSCQASGYTFT | 422 | DVVMTQTPLSLPVSLGDQASISCRSS | 525 |
| | NSDINWVKQRPGQGLEWIGWVYPRDGST | | QNIIHSNGDTYLDWFLQKPGQSPKLL | |
| | YYNEKFKAKATLTVDTSSSTAYMELYSLT | | IYKVSNRFSGVPDRFSGSGSGTDFTL | |
| | SEDSAVYFCARYAYALNYWGQGTSVTVS | | KISRVEAEDLGVYYCFQGSHVPYTFG | |
| | S | | GGTKLEIK | |
| TLA0187 | QIQLVQSGPELVKPGASVKLSCQASGYTFT | 423 | DVVMTQTPLSLPVSLGDQASISCRSS | 559 |
| | NSDINWVKQRPGQGLEWIGWVYPRDGRT | | QNIVHSNGDTYLEWYLQKPGQSPKL | |
| | YYNEKFKGKATLTVDTSSSTAYMELYSLT | | LIYKVSSRFSGVPDRFSGSGSGTDFTL | |
| | SEDSAVYFCARYAYALNSWGQGTSVTVSS | | KISRVEAEDLGIYYCFQGSHVPYTFG | |
| | | | GGTKLEIK | |
| TLA0188 | QIQLVQSGPELVKPGASVKLSCKASGYTFT | 424 | DVVMTQTPLSLPVSLGDQASISCRSS | 526 |
| | NSDINWVKQRPGQGLEWIGWVYPRDGST | | QSIVHSNGNTYLEWYLQKPGQSPKLL | |
| | YYNEKFKGKATLTVDTSSSTAYMELHSLT | | IYKVSNRFSGVPDRFSGSGSGTDFTL | |
| | SEDSAVYFCTRYAYALNSWGQGTSVTVSS | | KISRVEAEDLGVYYCFQGSHVPYTFG | |
| | | | RGDQAGNKT | |
| TLA0189 | QIQLVQSGPELVKPGASVKLSCKASGYTFT | 425 | DVVMTQTPLSLPVSLGDQASISCRSS | 558 |
| | NSDINWVKQRPGQGLEWIGWIYPRDGSTY | | QSIVHSNGNTYLEWYLQKPGQSPKLL | 5 |
| | YNEKFKGKATLTVDTSSTTAYMEFHSLTS | | IYKVSNRFSGVPDRFSGSGSGTDFTL | 5 |
| | EDSAVYFCARYAYTLNSWGQGTSVTVSS | | KISRVEAEDLGVYYCFQGSHVPYTFG | 8 |
| | | | GGTKLELK | |
| TLA0190 | EVQLQQSGAELARPGASVKMSCKASGYTF | 426 | DVVMTQTPLSLPVSLGDQASISCRSS | 527 |
| | TNSDINWVKQRPGQGLEWIGWIYPRDGST | | QSIVHSNGDTYLEWYLQKPGQSPKLL | |
| | YYNEKFKGKATLTVDTSSSTAYMELHSLT | | IYKVSNRFSGVPDRFSGSGSGTDFTL | |
| | SEDSAVYFCTRYAYALNSWGQGTSVTVSS | | KISRVEAEDLGIYYCFQGSHVPYTFG | |
| | | | GGTKLEIK | |
| TLA0191 | QVQLQQSGPELVKPGASVKLSCKASGYTF | 427 | DVVMTQTPLSLPVSLGDQASISCRSS | 553 |
| | TNSDINWVKQRPGQGLEWIGWIYPRDGST | | QSIVHSNGNTYLEWYLQKPGQSPKLL | |
| | YYNEKFKGKATLTVDTSSSTAYMELHSLT | | IYKVSNRFSGVPDRFSGSGSGTDFTL | |
| | SEDSAVYFCTRYAYALNSWGQGTSVTVSS | | KISRVEAEDLGVYYCFQGSHVPYTFG | |
| | | | GGTKLEIK | |
| TLA0192 | EVQLQQSGPELVKPGASVKLSCQASGYTF | 428 | DVVMTQTPLSLPVSLGDQASISCRSS | 538 |
| | TNSDINWVKQRPGQGLEWIGWVYPRDGR | | QNIVHSNGDTYLDWFLQKPGQSPKL | |
| | TYYNEKFKGKATLTVDTSSSTAYMELYSL | | LIYKVSNRFSGVPDRFSGSGSGTDFTL | |
| | TSEDSAVYFCARYAYALNSWGQGTSVTVS | | KISRVEAEDLGVYYCFQGSHVPYTFG | |
| | S | | GGTKLEIK | |
| TLA0193 | EVQLQQSGPELVRPGASVKLSCKASGYSF | 429 | DVVMTQTPLSLPVSLGNQASISCRSS | 528 |
| | TSGDINWVKQRPGQGLEWIAWIYPRDGST | | QDIVHTNGDTYLDWYLQKPGQSPKL | |
| | EYNEKFKGKATLTVDTSSSTAYMELHSLT | | LIYKVSNRFSGVPDRFSGSGSGTDFTL | |
| | SEDSAVYFCARYDYAFVYWGQGTLVTVS | | KINRVEAEDLGVYYCFQGSHVPYTF | |
| | A | | GGGTKLELK | |
| TLA0194 | EVQLQQSGPELVRPGTSVRLSCKTSGYTFT | 430 | DVVMTQTPLSLPVSLGDQASISCRSS | 529 |
| | SRDINWVKQRPGQGLEWIAWIYPRDGSTE | | QSIVHSNGDTYLDWFLQKPGQSPKLL | |
| | YNEKFKGKATLTVDTSSSTAYMELHSLTS | | IYKVSNRFSGVPDRFSGSGSGTDFTL | |
| | EDSAVYFCTRYDYAFTYWGQGTLVTVSA | | KISRVEAEDLGVYYCFQGSHVPYTFG | |
| | | | GGTKLELK | |
| TLA0195 | EVQLQQSGPEVVKPGASVKLSCKASGYTF | 431 | DVVMTQTPLYLPVSLGDQASISCRSS | 530 |
| | TRNDINWVKQRPGQGLEWIGWIYPRDGSS | | QSIVHSNGDTYLDWFLQKPGQSPKLL | |
| | KNNEKFKGKATLTVDTSSSTAYMELHSLT | | IYKVSNRFSGVPDRFSGSGSGTDFTL | |
| | SEDSAVYFCARYDYAFVYWGQGTTVTVS | | KISRVEAEDLGVYYCFQGSHVPYTFG | |
| | S | | GGTKLELK | |
| TLA0196 | EVQLQQSGPELVKPGASVKLSCKASGYTF | 432 | DVVMTQTPLSLPVSLGDQASISCRSS | 531 |
| | TSYDINWVKQRPGQGLEWIGWIYPRDGRT | | QTIVHSNGDTYLDWYLQKPGQSPKL | |
| | KYSEKFKGKATLTVDTSSSTAYMELHSLT | | LISKVSNRFSGVPDRFSGSGSGTDFTL | |
| | SEDSAVYFCARYGASMDYWGQGTSVTVS | | KISSVEAEDLGVYYCFQGSHVPYTFG | |
| | S | | GGTKLEIK | |
| TLA0197 | EVQLQQSGPELVKPGASVKLSCKASGYTF | 433 | DVVMTQTPLSLPVSLADQASISCRSS | 532 |
| | TSYDINWVKQSPGQGPEWIGWVYPRDGR | | QSIVHDNGDTYLEWYLQKPGQSPKL | |
| | TQYNEKFKGKATLTVDTSSSTAYMELHSL | | LIYKVSNRFSGVPDRFSGSGSGTDFTL | |
| | TSEDSAVYFCARYGASMDYWGQGTSVTV | | KISRVEAEDLGVYYCFQGSHVPYTFG | |
| | SS | | GGTKLEIK | |
| TLA0198 | EVQLQQSGPELAKPGASVKLSCKASGYTF | 434 | DVVMTQTPLSLPVSLGDQASISCRSS | 551 |
| | TSYDINWVKQRPGQGLEWIGWIYPRDGNT | | QNIVHSNGDTYLDWFLQKPGQSPKL | |
| | NYNEKFKGKATLTVDTSSNTASMELHSLT | | LIYKVSNRFSGVPDRFSGSGSGTDFTL | |
| | SEDSAVYFCARYSAAMDYWGQGTSVTVS | | KISRVEAEDLGVYYCFQGSHVPYTFG | |
| | S | | GGTKLELK | |
| TLA0199 | QVQLQQPGPELVKPGASVKLSCKASGYTS | 435 | DVVMTQTPLSLPVSLGDQASISCRSS | 533 |
| | TSYDINWVKQRPGQGLEWIGWIYPRDGRT | | QSIVHNNGDTYLDWYLEKPGQSPKL | |
| | NYNEKFKGKATLTVDTSSSTAFMELHSLT | | LIYKVSNRFSGVPDRFSGSGSGTDFTL | |
| | SEDSAVYFCARYSAAMDYWGQGTSVTVS | | KISRVEAEDLGVYYCFQGSHVPYTFG | |
| | S | | GGTKLEIK | |
| TLA0200 | QVQLQQSGPELVKPGASVKLSCKASGYTF | 436 | DVVMTQTPLSLPVSLGDQASISCRSS | 534 |
| | TSYDINWVKQRPGQGLEWIGWIFPRDGRT | | QNIVHNNGDTYLDWFLQKPGQSPKL | |
| | NYNEKFKDKATLTVDTSSSTAFMELHRLT | | LIYKVSNRFSGVPDRFSGSGSGTDFTL | |
| | SEDSAVYFCARYSAAMDYWGQGTSVTVS | | KISRVEAEDLGVYYCFQGSHVPYTFG | |
| | S | | GGTKLELK | |
| TLA0201 | EVQLQQSGAELGKPGASVKLSCKASGYTF | 437 | DVVMTQTPLSLPVSLGDQASISCKSS | 535 |
| | TNYDINWMKQRPGQGLEWIGWNYPRDGR | | QSIVHSNGNTYLEWYLQKPGQSPKLL | |
| | TKYNEKFKGKATLTVDTSSSTAYMELHSL | | IYKVSNRFSGVPDRFSGSGSGTDFTL | |
| | TSEDSAVYFCARYGYAMDYWGQGTSVTV | | KISRVEAEDLGVYYCFQGSHVPYTFG | |
| | SS | | GGTKLEIK | |
| TLA0202 | QVQLQQSGPDLVKPGASVKLSCKASGYIF | 438 | DVVMTQTPLSLPVSLGDQASISCRSS | 536 |
| | ASYDLNWVKQRPGQGLEWIGWIYPRDGR | | QNIVHSNGDTYLEWYLQKPGQSPKL | |
| | TDYNEKFKGKATLTVDTSSSTAYMELHSL | | LIYKVSNRFSGVPDRFSGSGSGTDFTL | |
| | TSEDSAVYFCARYGYALDYWGQGTSVTV | | KISRVEAEDLGVYYCFQGSYVPYTFG | |
| | SS | | GGTKLEIK | |
| TLA0203 | QIQLVQSGPELVKPGASVKVSCKASGYTFT | 439 | DVVMTQTPLSLPVSLGDQASISCRSS | 553 |
| | RQDINWVKQRPGQGLEWIGWIYPRDGRTE | | QSIVHSNGNTYLEWYLQKPGQSPKLL | |
| | YNEKFKGKATLTVDTSSSTAFMELHSLTSE | | IYKVSNRFSGVPDRFSGSGSGTDFTL | |
| | DSAVYFCARYSAAMDYWGQGTSVTVSS | | KISRVEAEDLGVYYCFQGSHVPYTFG | |
| | | | GGTKLEIK | |
| TLA0204 | QIQLVQSGPELVKPGASVKLSCKASGYTFT | 440 | DVVMTQTPLSLPVSLGDQASISCRSS | 558 |
| | NSDINWVKQRPGQGLEWIGWNYPRDGRT | | QSIVHSNGNTYLEWYLQKPGQSPKLL | |
| | KYNEKFKGKATFTVDTSSSTAYMDLQSLT | | IYKVSNRFSGVPDRFSGSGSGTDFTL | |
| | SEDSAVYFCTRYGSAMDYWGQGTSVTVS | | KISRVEAEDLGVYYCFQGSHVPYTFG | |
| | S | | GGTKLELK | |
| TLA0205 | EVQLQQSGPELVKPGASVKLSCKASGYTF | 441 | DVVMTQTPLSLPVSLGDQASISCRSS | 551 |
| | TNQDINWVKQRPGQGLEWIGWIFPRDGRT | | QNIVHSNGDTYLDWFLQKPGQSPKL | |
| | HYNENFKGKATLTVDTSSSTAYMELHSLT | | LIYKVSNRFSGVPDRFSGSGSGTDFTL | |
| | AEDSAVYFCAIYGIALDHWGQGTSVTVSS | | KISRVEAEDLGVYYCFQGSHVPYTFG | |
| | | | GGTKLELK | |
| TLA0206 | QVQLQQSGPELVKPGASVKLSCKASGYTF | 442 | DVVMTQTPLSLPVSLGDQASISCRSS | 553 |
| | TNQDINWVKQRPGQGLEWIGWIFPRDGRT | | QSIVHSNGNTYLEWYLQKPGQSPKLL | |
| | HYNENFKGKATLTVDTSSNTAYMELHSLT | | IYKVSNRFSGVPDRFSGSGSGTDFTL | |
| | AEDSAVYFCAIYGIALDHWGQGTSVTVSS | | KISRVEAEDLGVYYCFQGSHVPYTFG | |
| | | | GGTKLEIK | |
| TLA0207 | QVQLQQSGPELVKPGASVKLSCKASGYTF | 443 | DVVMTQTPLSLPVSLGDQASISCRSS | 537 |
| | TNQDINWVKQRPGQGLEWIGWIFPRDGRT | | QNIVHSNGDTYLEWYLQKPGQSPKL | |
| | HYNENFKGKATLTVDTSSSTAYMELHSLT | | LIYKVSNRFSGVPDRFSGSGSGTDFTL | |
| | AEDSAVYFCAIYGIALDHWGQGTSVTVSS | | KISRVEAEDLGVYYCFQGSHVPYTFG | |
| | | | GGTKLEIK | |
| TLA0208 | QVQLQQSGPELVKPGASVKLSCKASGYTF | 444 | DVVMTQTPLSLPVSLGDQASISCRSS | 553 |
| | TNQDINWVKQRPGQGLEWIGWIFPRDGRT | | QSIVHSNGNTYLEWYLQKPGQSPKLL | |
| | HYNENFKGKATLTVDTSSSTAYMELHSLT | | IYKVSNRFSGVPDRFSGSGSGTDFTL | |
| | AEDSAVYFCAIYGAAMDHWGQGTSVTVS | | KISRVEAEDLGVYYCFQGSHVPYTFG | |
| | S | | GGTKLEIK | |
| TLA0209 | QVQLQQSGPELVKPGTSVKLSCKASGYTF | 445 | DVVMTQTPLSLPVSLGDQASISCRSS | 538 |
| | TNQDINWVKQRPGQGLEWIGWIFPRDGRT | | QNIVHSNGDTYLDWFLQKPGQSPKL | |
| | HYNENFKGKATLTVDTSSSTAYMELHSLT | | LIYKVSNRFSGVPDRFSGSGSGTDFTL | |
| | AEDSAVYFCSIYGIAMDHWGQGTSVTVSS | | KISRVEAEDLGVYYCFQGSHVPYTFG | |
| | | | GGTKLEIK | |
| TLA0210 | QVQLQQPGPELVKPGASVKLSCKASGYTF | 446 | DVVMTQTPLSLPVSLGDQASISCRSS | 539 |
| | TNQDINWVKQRPGQGLEWIGWIFPRDGRT | | QSIVHSNGDTYLDWFLQKPGQSPKLL | |
| | HNNENFKGKATLTVDTSSSTAYMELHSLT | | IYKVSNRFSGVPDRFSGSGSGTDFTL | |
| | AEDSAVYFCAIYGFAMDHWGQGTSVTVS | | KISRVESEDLGVYYCFQGSHDPYTFG | |
| | S | | GGTKLEIK | |
| TLA0211 | QVQLQQSGPELVKPGASVKLSCKASGYTF | 447 | DVVMTQTPLSLPVSLGDQASISCRSS | 558 |
| | TNQDINWVKQRPGQGLEWIGWIFPRDGRT | | QSIVHSNGNTYLEWYLQKPGQSPKLL | |
| | HYNENFKGKATLTVDTSSSTAYMELHSLT | | IYKVSNRFSGVPDRFSGSGSGTDFTL | |
| | AEDSAVYFCAIYGFAMDHWGQGTSVTVS | | KISRVEAEDLGVYYCFQGSHVPYTFG | |
| | S | | GGTKLELK | |
| TLA0212 | QVQLQQPGPELVKPGASVKLSCKASGYTF | 448 | DVVMTQTPLSLPVSLGDQASISCRSS | 540 |
| | TNQDINWVKQRPGQGLEWIGWIFPRDGRT | | QSIVHSNGNTYLEWYLQKPGQSPKLL | |
| | HYNENFKGKATLTVDTSSSTAYMELHSLT | | IYKVSDRFSGVPDRFSGSGSGTDFTL | |
| | AEDSAVYFCAIYGAAMDDWGQGTSVTVS | | KISRVEAEDLGVYYCFQGSHVPYTFG | |
| | S | | GGTKLEIK | |
| TLA0213 | QVQLQQPGPELVKPGASVKLSCKASGYTF | 449 | DVVMTQTPLSLPVSLGHQASISCRSS | 541 |
| | TNQDINWVKQRPGQGLEWIGWIFPRDGRT | | QSIVHSNGNTYLEWYLQKPGQSPKLL | |
| | HYNENFKGKATLTVDTSSSTAFMELHSLT | | IYKVSNRFSGVPDRFSGSGSGTDFTL | |
| | AEDSAVYFCAIYGIALDHWGQGTSVTVSS | | KISRVEAEDLGVYYCFQGSHVPYTFG | |
| | | | GGTKLELK | |
| TLA0213 | EVQLQQSGPELVKPGASVKLSCKASGYTF | 450 | DVVMTQTPLSLPVSLGDQASISCRSS | 553 |
| | TNQDINWVKQRPGQGLEWIGWIFPRDGRT | | QSIVHSNGNTYLEWYLQKPGQSPKLL | |
| | HYNENFKGKATLTVDTSSSTAYMELHSLT | | IYKVSNRFSGVPDRFSGSGSGTDFTL | |
| | AEDSAVYFCAIYGFAMDHWGQGTSVTVS | | KISRVEAEDLGVYYCFQGSHVPYTFG | |
| | S | | GGTKLEIK | |
| TLA0214 | EVQLQQSGPELVKPGASVKLSCKASGYTF | 451 | DVVMTQTPLSLPVSLGDQASISCRSS | 558 |
| | TNQDINWVKQRPGQGLEWIGWNFPRDGR | | QSIVHSNGNTYLEWYLQKPGQSPKLL | |
| | THYNENFKGKATLTVDTSSSTAYMELHSL | | IYKVSNRFSGVPDRFSGSGSGTDFTL | |
| | TAEDSAVFFCAIYGFALDHWGQGTSVTVS | | KISRVEAEDLGVYYCFQGSHVPYTFG | |
| | S | | GGTKLELK | |
| TLA0216 | QVQLQQSGPELVKPGASVKLSCKASGYTF | 452 | DVVMTQTPLSLPVSLADQASISCRSS | 542 |
| | TNQDINWVKQRPGQGLEWIGWIFPRDGRT | | QSIVHDNGDTYLEWYLQKPGQSPKL | |
| | HYNENFKGKATLTVDTSSSTAYMELHSLT | | LIYKVSNRFSGVPDRFSGSGSGTDFTL | |
| | AEDSAVYFCALYGFAMDQWGQGTSVTVS | | KISRVEAEDLGVYYCFQGSHVPYTFG | |
| | S | | WRDQAGNKT | |
| TLA0217 | EVQLQQSGPELVKPGASVKLSCKASGYTF | 453 | DVVMTQTPLSLPVSLGDQASISCRSS | 513 |
| | TNQDINWVKQRPGQGLEWIGWIFPRDGRT | | QSIVHSNGNTYLEWYLQKPGQSPKLL | |
| | HYNEKFKGKATLTVDTSSSTAYMELHSLT | | IYKASNRFSGVPDRFSGSGSGTDFTLR | |
| | AEDSAVYFCAIYGSAMDQWGQGTSVTVS | | ISSVEAEDLGVYYCFQGSHVPYTFGG | |
| | S | | GTKLEIK | |
| TLA0218 | EVQLQQSGPELVKPGTSVKIPCKTSGYTFT | 454 | DVVMTQTPLSLPVSLGDQASISCRSS | 544 |
| | DYNINWVKQSHGKSLEWIGDINPDSGGTV | | QSIVHSNGNTYLEWYLQKPGQSPKLL | |
| | YNQRFKDKATLTVDKSSSTAYMELRSLTS | | IYKVSNRFSGVPDRFSGSGSGTDFTL | |
| | GDTAVYYCARWGYYDGRGFDYWGQGTT | | KISRVEAEDLGVYYCFQGSHVPLTFG | |
| | LTVSS | | AGTKLELK | |
| TLA0219 | EVQLQQSGAELVRPGASVTLSCKASGYTF | 455 | DVVMTQTPLSLPVSLGDQASISCRSS | 545 |
| | TDYEIHWVKQIPVHGLEWIGVFDPETGGA | | QSLVHSNGNTYLHWYLQKPGQSPKL | |
| | AYNQKFKGKAILTADKSSSTAYMELRSLT | | LIYKVSNRFSGVPDRFSGSGSGTDFTL | |
| | SEDSAVYYCTTPWLNYFEYWGQGTTLTVS | | KISRVEAEDLGVYFCSQSTHVPFTFGS | |
| | S | | GTKLEIK | |
| TLA0220 | QVQLQQSVAELVRPGASVKLSCTASGFNIE | 456 | EIVLTQSPALMSASPGEKVTITCSASS | 546 |
| | DTYMHWVKQRPEQGLEWIGRVDPASGNT | | SVSYMHWFQQKPGTSPKLWIYSTSN | |
| | KYGPKFQGKATITADTSSNTAYLQLSSLSS | | LASGVPARFSGSGSGTSYSLTISRMEA | |
| | ADTAIYYCSTWGLFDYWGQGTTLTVSS | | EDAATYYCQQRSSYPFTFGSGTKLEI | |
| | | | K | |
| TLA0221 | QVQLQQPGPELVKPGASVKLSCKASGYTF | 457 | DVVMTQTPLSLPVSLGDQASISCRSS | 547 |
| | TSIDINWVKQRPGQGLEWIGWIYPRDGRT | | QSIVHSNGNTYLEWYLQKPGQSPKLL | |
| | HYNEKFKGKATLTVDTSSSTAYMELHSLT | | IYKVSNRFSGVPDRFSGSGSGTDFTL | |
| | SEDSAVYFCARYTSSLDYWGQGTTLTVSS | | KISRVEAEDLGVYYCFQGSHVPYTFG | |
| | | | GGTKLEIKR | |
| TLA0222 | QVQLQQSGPELVKPGASVKLSCKASGYTF | 458 | DVVMTQTPLSLPVSLGDQASISCRSS | 550 |
| | TSIDINWVKQRPGQGLEWIGWIYPRDGRT | | QSIVHSNGDTYLDWFLQKPGQSPKLL | |
| | DYNEKFKGKATLTVDTSSSTAYMELHSLT | | IYKVSNRFSGVPDRFSGSGSGTDFTL | |
| | SEDSAVYFCARYGSSLDYWGQGTTLTVSS | | KISRVEAEDLGVYYCFQGSHVPYTFG | |
| | | | GGTKLEIK | |
| TLA0223 | QVQLQQSGPELVKPGTSVRLSCKASGYTF | 459 | DVVMTQTPLSLPVSLGDQASISCRSS | 548 |
| | TNSDINWVKQRPGQGLEWIGWIYPRDGRT | | QNIVHSNGDTYLDWYLQKPGQSPKL | |
| | DYNEKFKGKATLTVDTSSSTAYMELHSLT | | LISKVSNRFSGVPDRFSGSGSGTDFTL | |
| | SEDSAVYFCARYGSSLYWGQGTTLTVSS | | KISRVEAEDLGVYYCFQGSHVPYTFG | |
| | | | GGTKLEIK | |
| TLA0224 | EVQLQQSGPELVKPGASVKLSCKASGYTF | 460 | DVVMTQTPLSLPVSLGDQASISCRSS | 549 |
| | TSSDINWVKQRPGQGPEWIGWIYPRDGRT | | QNIVHSNGDTYLDWFLQKPGQSPKL | |
| | HYNEKFKGKATLTVDTSSSTAYMELHSLT | | LISKVSNRFSGVPDRFSGSGSGTDFTL | |
| | SEDSAVYFCAPYGSSLAYWGQGTTLTVSS | | KISRVEAEDLGVYYCFQGSHVPYTFG | |
| | | | GGTKLEIK | |
| TLA0225 | QIQLVQSGPELVKPGASVKLSCKASGYTFT | 461 | DVVMTQTPLSLPVSLGDQASISCRSS | 550 |
| | SSDINWVKQRPGQGLEWIGWIYPRDGSTH | | QSIVHSNGDTYLDWFLQKPGQSPKLL | |
| | YNENFKGKATLTVDTSSSTAYMELHSLTS | | IYKVSNRFSGVPDRFSGSGSGTDFTL | |
| | EDSAVYFCAPYGSSLAYWGQGTTLTVSS | | KISRVEAEDLGVYYCFQGSHVPYTFG | |
| | | | GGTKLEIK | |
| TLA0226 | QVQLKESGPEVVKPGASVKLSCKASGYTF | 462 | DVVMTQTPLSLPVSLGDQASISCRSS | 551 |
| | TSYDINWVKQRPGQGLEWIGWIYPRDGRT | | QNIVHSNGDTYLDWFLQKPGQSPKL | |
| | EYNEKFKDKATLTVDTSSSTAYMELHSLT | | LIYKVSNRFSGVPDRFSGSGSGTDFTL | |
| | SEDSAVYFCARYGSSLDYWGQGTTLTVSS | | KISRVEAEDLGVYYCFQGSHVPYTFG | |
| | | | GGTKLELK | |
| TLA0227 | QIQLVQSGPELVKPGASVKVSCKASGYTFT | 463 | DVVMTQTPLSLPVSLADQASISCRSS | 552 |
| | RQDINWVKQRPGQGLEWIGWIYPRDGSTK | | QSIVHDNGDTYLEWYLQKPGQSPRL | |
| | YNEKFKGKVTLTVDTSSSTAYMELHSLTS | | LIYKVSNRFSGVPDRFSGSGSGTDFTL | |
| | EDSAVYFCATYGSSLDNWGQGTTLTVSS | | KINRVEAEDLGVYYCFQGSHVPYTF | |
| | | | GGGTKLEIK | |
| TLA0228 | QVQLQQSGPELVKPGASVKVSCKASGYTF | 464 | DVVMTQTPLSLPVSLGDQASISCRSS | 553 |
| | TRQDINWVKQRPGQGLEWIGWIYPRDGST | | QSIVHSNGNTYLEWYLQKPGQSPKLL | |
| | KYNEKFKGKVTLTVDTSSSTAYMELHSLT | | IYKVSNRFSGVPDRFSGSGSGTDFTL | |
| | SEDSAVYFCATYGSSLDNWGQGTTLTVSS | | KISRVEAEDLGVYYCFQGSHVPYTFG | |
| | | | GGTKLEIK | |
| TLA0229 | QIQLVQSGPELVKPGASVKVSCKASGYTFT | 465 | DVVMTQTPLSLPVSLGDQASISCRSS | 554 |
| | RQDINWVKQRPGQGLEWIGWIYPRDGRTE | | QSIVHNNGDTYLEWYLQKPGQSPKL | |
| | YNEKFKGKVTLTVDTSSSTAYMELHSLTP | | LIYKVSNRFSGVPDRFSGSGSGTDFTL | |
| | EDSAVYFCATYGSSLDNWGQGTTLTVSS | | KISRVEAEDLGVYYCFQGSHVPYTFG | |
| | | | GGTKLEIK | |
| TLA0230 | QVQLKESGPELVKPGASVKVSCKASGYTF | 466 | DVVMTQTPLSLPVSLGDQASISCRSS | 555 |
| | TRQDINWVKQRPGQGLEWIGWIYPRDGRT | | QNIVHTNGDTYLDWYLQKPGQSPKL | |
| | EYNEKFKGKVTLTVDTSSSTAYMELHSLT | | LIYKVSNRFSGVPDRFSGSGSGTDFTL | |
| | SEDSAVYFCATYGSSLDNWGQGTTLTVSS | | KISRVEAEDLGVYYCFQGSHVPYTFG | |
| | | | GGTKLELK | |
| TLA0231 | QVQLQQSGAELAKPGASVKLSCKASGYTF | 467 | DVVMTQTPLSLPVSLGDQASISCRSS | 556 |
| | TRQDINWVKQRPGQGLEWIGWIYPRDGRT | | QTIVHSNGNTYLDWYLQKPGHSPQL | |
| | EYNEKFKGKVTLTVDTSSSTAYMELHSLT | | LIYKVSNRFSGVPDRFSGSGSGTDFTL | |
| | SEDSAVYFCATYGSSLDNWGQGTTLTVSS | | KISRVEAEDLGVYYCFQGSHVPYTFG | |
| | | | GGTKLEIK | |
| TLA0232 | QVQLQQPGPELVKPGASVKVSCKASGYTF | 468 | DVVMTQTPLSLPVSLGDQASISCRSS | 557 |
| | TRQDINWVKQRPGQGLEWIGWIYPRDGRT | | QSIVHSNGNTYLEWYLQKPGQSPKLL | |
| | EYNEKFKGKVTLTVDTSSNTAYMELHSLT | | IYKVSNRFSGVPDRFSGSGSGTDFTL | |
| | SEDSAVYFCATYGSSLDNWGQGTTLTVSS | | KISRVEAEDLGVYYCFQGSHVPYTFG | |
| | | | SGTKLEIK | |
| TLA0233 | QVQLQQSGPELVKPGASVKVSCKASGYTF | 469 | DVVMTQTPLSLPVSLGDQASISCRSS | 558 |
| | TRQDINWVKQRPGQGLEWIGWIYPRDGRT | | QSIVHSNGNTYLEWYLQKPGQSPKLL | |
| | EYNEKFKGKVTLTVDTSSSTAYMELHSLT | | IYKVSNRFSGVPDRFSGSGSGTDFTL | |
| | SEDSAVYFCATYGSSLDNWGQGTTLTVSS | | KISRVEAEDLGVYYCFQGSHVPYTFG | |
| | | | GGTKLELK | |
| TLA0234 | EVQLQQSGPELVKPGASVKVSCKASGYTF | 470 | DVVMTQTPLSLPVSLGDQASISCRSS | 558 |
| | TRQDINWAKQRPGQGLEWIGWIYPRDGRT | | QSIVHSNGNTYLEWYLQKPGQSPKLL | |
| | EYNEKFKGKVTLTVDTSSSTAYMELHSLT | | IYKVSNRFSGVPDRFSGSGSGTDFTL | |
| | PEDSAVYFCATYGSSLDNWGQGTTLTVSS | | KISRVEAEDLGVYYCFQGSHVPYTFG | |
| | | | GGTKLELK | |
| TLA0235 | EVQLQQSGPELVKPGASVKVSCKASGYTF | 471 | DVVMTQTPLSLPVSLGDQASISCRSS | 559 |
| | TRQDINWVKQRPGQGLEWIGWIYPRDGRT | | QNIVHSNGDTYLEWYLQKPGQSPKL | |
| | EYNEKFKGKVTLTVDTSSSTAYMELHSLT | | LIYKVSSRFSGVPDRFSGSGSGTDFTL | |
| | SEDSAVYFCATYGSSLDNWGQGTTLTVSS | | KISRVEAEDLGIYYCFQGSHVPYTFG | |
| | | | GGTKLEIK | |
| TLA0236 | EVQLQQSVAELVRPGASVKLSCTASGFNIE | 472 | EILLTQSPAIIAASPGEKVTITCSASSS | 560 |
| | NTYIHWVKQRPEQGLEWIGRIDPASGNTK | | VSYMNWYQQKPGSSPKIWIYGISNLA | |
| | YAPKFQGKATITADTSSNTAYLQLSSLTSE | | SGVPARFSGSGSGTSFSFTINSMEAED | |
| | DTAIYYCASTGPDYWGQGTTLTVSS | | VATYYCQQRSSYPFTFGSGTKLEIK | |
| TLA0237 | QIQLVQSGPELKKPGETVEISCKASGYTFT | 473 | DVLMTQAPLSLPVSLGDQASISCRSN | 561 |
| | THGMNWVKQAPGKGLKWMGWINTFSGV | | QTIVHSNGNTYLEWYLQKPGQSPKL | |
| | PTYADDFRGRFDFFLATSASTAYLQINNLK | | LIYKVSNRFSGVPDRFSGSGSGTDFTL | |
| | NEDTATYFCARDYGTAYAMDSWGQGTSV | | KISGVEAEDLGVYYCFQGSHVPLTFG | |
| | TVSS | | AGTKLELN | |
| TLA0238 | EVKVEESGGGLVQPGGSMKLSCIASGFTFS | 474 | ENVLTQSPAIMSASPGEKVTMTCSAS | 562 |
| | NYWMNWVRQSPEKGLEWVAQIRLKSDN | | SSVSYMHWYQQKSSTSPKLWIYDTS | |
| | YATHYAESVKGNFTISRDDSKSSVYLQMN | | NLASGVPGRFSGSGSGNSYSLTISSME | |
| | NLRAEDTGIYYCTPLLLRYRDYWGQGTTL | | AEDVATYYCFQENGHPFTFGGGTKL | |
| | TVSS | | EMK | |
| TLA0239 | EVQLQQSGPELVKPGASVKISCKASGYTFT | 475 | DILMTQSPSSMSVSLGDTVSITCHASQ | 563 |
| | DYYMNWVKQSHGKSLEWIGDVNPNSGGT | | DIFSYIGWLQQKPGKSFKGLIHHGTN | |
| | SQNQKFKGKATLTVDKSSTTAYMELRSLT | | LEDGVPSRFRGNGSGADYSLTISSLES | |
| | SEDSAVYHCARGSNYVGGFTYWGQGTLV | | EDFADYYCVQYAQFPYTFGGGTKLEI | |
| | AVSA | | K | |
| TLA0240 | QVQLQQPGAELVKPGASVKLSCKASGYTF | 476 | DIVMTQSPSSLRVSAGEKVTMSCKSS | 564 |
| | SNDWMHWVKQRPGRGLEWIGRIDPDSGG | | QSLLNSGNQKNYLAWYQQKPGQPPK | |
| | TKYNEKFKNKATLTVDKPSSTVYMQLSSL | | LLIYGASTRESGVPDRFTGSGSGTDFT | |
| | TSEDSAVYYCARSEGYYGSRSYFDYWGQ | | LTISSVQAEDLAVYYCQNDHSYPYTF | |
| | GTTLTVSS | | GGGTKLEIK | |
| TLA0241 | EFQLQQSGPELVKPGASVRISCKASGYTFT | 477 | EIVLTQSPTTMAASPGEKITITCSTSSS | 565 |
| | DYYLNWVKQSRGESLEWIGDINPNNGRTT | | IISNYSHWYQQKPGFSPKLLIYKTSNL | |
| | YNQKFTGKATWTVDKSSSTVYMELRSLTS | | PSGVPARFSGSGSGTSYSLTIGTMEAE | |
| | DDSAVYYCAREGGYGWYFDVWGTGTTV | | DAATYYCQQGSDMPITFGAGTKLEL | |
| | TVSS | | K | |

The single underlined line represents the sequence of CDR1, CDR2, and CDR3. CDRs are defined by Kabat.

### Example 4 Humanized Design and Identification of Anti-TL1A Murine Monoclonal Antibody

4.1 TLA0015 humanized design: according to the VH and VL amino acid sequences of TLA0015 murine monoclonal antibodies, the IGHV3-23/IGHV3-39/IGKV1-39 Germline sequences which has the highest homology in the human germline database were selected as templates, and the CDR regions of murine antibodies were transplanted to the corresponding humanized templates to obtain humanized sequences, and the heavy and light chain pairs were paired to obtain TLA0015 humanized antibody sequence pairs, the specific sequences are shown in Table 3 below

TABLE 3
TLA0015 humanized antibody amino acid sequences

| TLA0015 | | | | |
|---|---|---|---|---|
| humanized | | SEQ | | SEQ |
| antibody | | ID | | ID |
| sequence | Amino acid sequence (VH) | NO | Amino acid sequence (VL) | NO |
| TLA0015 | QIQLKESGPGLVAPSQSLSITCTVSGFSL | 62 | DIQMTQTTSSLSASLGDRVTISCRA | 102 |
| | SSYGVDWVRQSPGKGLEWLGVIWGFG | | SQDISNYLNWYQQKPDGTVKLLIY | |
| | GTNYNSALKSRLSISKDNSKSQVFLKM | | YTSRLHSGVPSRESGSGSGTDYSL | |
| | NSLQSDDSAIYYCASGNFDAMDYWGQ | | TISNLEQEDIATYFCQQGNTLPFTF | |
| | GTSVTVSS | | GSGTKLEIK | |
| TLA0015-1 | EIQLLESGGGLVQPGGSLRLSCAVSGFS | 63 | DIQMTQSPSSLSASVGDRVTITCRA | 103 |
| | LSSYGVDWVRQAPGKGLEWVGVIWGF | | SQDISNYLNWYQQKPGKVVKLLI | |
| | GGTNYNSALKSRLTISRDNSKNTLYLQ | | YYTSRLHSGVPSRFSGSGSGTDYT | |
| | MNSLRAEDTAVYYCASGNFDAMDYW | | LTISSLQPEDVATYFCQQGNTLPFT | |
| | GQGTLVTVSS | | FGPGTKLEIK | |
| TLA0015-2 | EIQLLESGGGLVQPGGSLRLSCAVSGFS | 63 | DIQMTQSPSSLSASVGDRVTITCRA | 106 |
| | LSSYGVDWVRQAPGKGLEWVGVIWGF | | SQDISNYLNWYQQKPGKAVKLLI | |
| | GGTNYNSALKSRLTISRDNSKNTLYLQ | | YYTSRLHSGVPSRFSGSGSGTDYT | |
| | MNSLRAEDTAVYYCASGNFDAMDYW | | LTISSLQPEDFATYFCQQGNTLPFT | |
| | GQGTLVTVSS | | FGQGTKLEIK | |
| TLA0015-3 | EIQLLESGGGLVQPGGSLRISCAVSGFS | 64 | DIQMTQSPSSLSASVGDRVTITCRA | 103 |
| | LSSYGVDWVRQAPGKGLEWLGVIWGF | | SQDISNYLNWYQQKPGKVVKLLI | |
| | GGTNYNSALKSRLTISKDNSKNTVYLQ | | YYTSRLHSGVPSRFSGSGSGTDYT | |
| | MNSLRAEDTAVYYCASGNFDAMDYW | | LTISSLQPEDVATYFCQQGNTLPFT | |
| | GQGTLVTVSS | | FGPGTKLEIK | |
| TLA0015-4 | EIQLLESGGGLVQPGGSLRISCAVSGFS | 64 | DIQMTQSPSSLSASVGDRVTITCRA | 106 |
| | LSSYGVDWVRQAPGKGLEWLGVIWGF | | SQDISNYLNWYQQKPGKAVKLLI | |
| | GGTNYNSALKSRLTISKDNSKNTVYLQ | | YYTSRLHSGVPSRFSGSGSGTDYT | |
| | MNSLRAEDTAVYYCASGNFDAMDYW | | LTISSLQPEDFATYFCQQGNTLPFT | |
| | GQGTLVTVSS | | FGQGTKLEIK | |
| TLA0015-5 | EIQLVESGGGLVQPGGSLRISCAVSGFS | 65 | DIQMTQSPSSLSASVGDRVTITCRA | 103 |
| | LSSYGVDWVRQSPGKGLEWLGVIWGF | | SQDISNYLNWYQQKPGKVVKLLI | |
| | GGTNYNSALKSRLTISKDNSKNTVYLQ | | YYTSRLHSGVPSRFSGSGSGTDYT | |
| | MNSLRAEDTAVYYCASGNFDAMDYW | | LTISSLQPEDVATYFCQQGNTLPFT | |
| | GQGTLVTVSS | | FGPGTKLEIK | |
| TLA0015-6 | EIQLVESGGGLVQPGGSLRISCAVSGFS | 65 | DIQMTQSPSSLSASVGDRVTITCRA | 106 |
| | LSSYGVDWVRQSPGKGLEWLGVIWGF | | SQDISNYLNWYQQKPGKAVKLLI | |
| | GGTNYNSALKSRLTISKDNSKNTVYLQ | | YYTSRLHSGVPSRFSGSGSGTDYT | |
| | MNSLRAEDTAVYYCASGNFDAMDYW | | LTISSLQPEDFATYFCQQGNTLPFT | |
| | GQGTLVTVSS | | FGQGTKLEIK | |
| TLA0015-7 | EVQLLESGGGLVQPGGSLRLSCAVSGF | 66 | DIQMTQSPSSLSASVGDRVTITCRA | 106 |
| | SLSSYGVDWVRQAPGKGLEWLGVIWG | | SQDISNYLNWYQQKPGKAVKLLI | |
| | FGGTNYNSALKSRFTISRDNSKNTVYL | | YYTSRLHSGVPSRFSGSGSGTDYT | |
| | QMNSLRAEDTAVYYCASGNFDAMDY | | LTISSLQPEDFATYFCQQGNTLPFT | |
| | WGQGTLVTVSS | | FGQGTKLEIK | |
| TLA0015-8 | EVQLLESGGGLVQPGGSLRLSCAVSGF | 67 | DIQMTQSPSSLSASVGDRVTITCRA | 106 |
| | SLSSYGVDWVRQAPGKGLEWLGVIWG | | SQDISNYLNWYQQKPGKAVKLLI | |
| | FGGTNYNSALKSRLTISRDNSKNTVYL | | YYTSRLHSGVPSRFSGSGSGTDYT | |
| | QMNSLRAEDTAVYYCASGNFDAMDY | | LTISSLQPEDFATYFCQQGNTLPFT | |
| | WGQGTLVTVSS | | FGQGTKLEIK | |
| TLA0015-9 | EVQLLESGGGLVQPGGSLRLSCAVSGF | 68 | DIQMTQSPSSLSASVGDRVTITCRA | 106 |
| | SLSSYGVDWVRQAPGKGLEWLGVIWG | | SQDISNYLNWYQQKPGKAVKLLI | |
| | FGGTNYNSALKSRLTISKDNSKNTVYL | | YYTSRLHSGVPSRFSGSGSGTDYT | |
| | QMNSLRAEDTAVYYCASGNFDAMDY | | LTISSLQPEDFATYFCQQGNTLPFT | |
| | WGQGTLVTVSS | | FGQGTKLEIK | |
| TLA0015-10 | EIQLVESGGGLIQPGGSLRLSCAVSGFS | 69 | DIQMTQSPSSLSASVGDRVTITCRA | 106 |
| | LSSYGVDWVRQAPGKGLEWLGVIWGF | | SQDISNYLNWYQQKPGKAVKLLI | |
| | GGTNYNSALKSRLTISRDNSKNTVYLQ | | YYTSRLHSGVPSRFSGSGSGTDYT | |
| | MNSLRAEDTAVYYCARGNFDAMDYW | | LTISSLQPEDFATYFCQQGNTLPFT | |
| | GQGTLVTVSS | | FGQGTKLEIK | |
| TLA0015-11 | EIQLVESGGGLIQPGGSLRISCAVSGESL | 70 | DIQMTQSPSSLSASVGDRVTITCRA | 106 |
| | SSYGVDWVRQAPGKGLEWLGVIWGFG | | SQDISNYLNWYQQKPGKAVKLLI | |
| | GTNYNSALKSRLTISKDNSKNTVYLQM | | YYTSRLHSGVPSRFSGSGSGTDYT | |
| | NSLRAEDTAVYYCASGNFDAMDYWG | | LTISSLQPEDFATYFCQQGNTLPFT | |
| | QGTLVTVSS | | FGQGTKLEIK | |
| TLA0015-12 | EIQLVESGGGLIQPGGSLRISCAVSGESL | 70 | DIQMTQSPSSLSASVGDRVTITCRA | 104 |
| | SSYGVDWVRQAPGKGLEWLGVIWGFG | | SQDISNYLNWYQQKPGKAPKLLIY | |
| | GTNYNSALKSRLTISKDNSKNTVYLQM | | YTSRLHSGVPSRFSGSGSGTDFTLT | |
| | NSLRAEDTAVYYCASGNFDAMDYWG | | ISSLQPEDFATYYCQQGNTLPFTFG | |
| | QGTLVTVSS | | QGTKLEIK | |
| TLA0015-13 | EIQLVESGGGLIQPGGSLRISCAVSGESL | 70 | DIQMTQSPSSLSASVGDRVTITCRA | 105 |
| | SSYGVDWVRQAPGKGLEWLGVIWGFG | | SQDISNYLNWYQQKPGKAPKLLIY | |
| | GTNYNSALKSRLTISKDNSKNTVYLQM | | YTSRLHSGVPSRESGSGSGTDYTL | |
| | NSLRAEDTAVYYCASGNFDAMDYWG | | TISSLQPEDFATYFCQQGNTLPFTF | |
| | QGTLVTVSS | | GQGTKLEIK | |
| TLA0015-14 | EVQLLESGGGLVQPGGSLRLSCAVSGF | 66 | DIQMTQSPSSLSASVGDRVTITCRA | 104 |
| | SLSSYGVDWVRQAPGKGLEWLGVIWG | | SQDISNYLNWYQQKPGKAPKLLIY | |
| | FGGTNYNSALKSRFTISRDNSKNTVYL | | YTSRLHSGVPSRFSGSGSGTDFTLT | |
| | QMNSLRAEDTAVYYCASGNFDAMDY | | ISSLQPEDFATYYCQQGNTLPFTFG | |
| | WGQGTLVTVSS | | QGTKLEIK | |
| TLA0015-15 | EVQLLESGGGLVQPGGSLRLSCAVSGF | 67 | DIQMTQSPSSLSASVGDRVTITCRA | 104 |
| | SLSSYGVDWVRQAPGKGLEWLGVIWG | | SQDISNYLNWYQQKPGKAPKLLIY | |
| | FGGTNYNSALKSRLTISRDNSKNTVYL | | YTSRLHSGVPSRFSGSGSGTDFTLT | |
| | QMNSLRAEDTAVYYCASGNFDAMDY | | ISSLQPEDFATYYCQQGNTLPFTFG | |
| | WGQGTLVTVSS | | QGTKLEIK | |
| TLA0015-16 | EVQLLESGGGLVQPGGSLRLSCAVSGF | 66 | DIQMTQSPSSLSASVGDRVTITCRA | 105 |
| | SLSSYGVDWVRQAPGKGLEWLGVIWG | | SQDISNYLNWYQQKPGKAPKLLIY | |
| | FGGTNYNSALKSRFTISRDNSKNTVYL | | YTSRLHSGVPSRFSGSGSGTDYTL | |
| | QMNSLRAEDTAVYYCASGNFDAMDY | | TISSLQPEDFATYFCQQGNTLPFTF | |
| | WGQGTLVTVSS | | GQGTKLEIK | |

Where single underline represents the sequence of FR1, FR2, FR3, FR4, and the rest portion is CDR1, CDR2, CDR3 in that order. CDRs are defined by Kabat.

TLA0015-11 was mutated to improve the active function of TLA0015-11-related antibodies, and 31 antibodies were designed and obtained, and the specific amino acid sequences are shown in Table 4.

TABLE 4

| | | SEQ | | SEQ |
|---|---|---|---|---|
| Antibody | | ID | | ID |
| NO. | VH | NO | VL | NO |
| TLa0015- | EIQLVESGGGLIQPGGSLRISCAVSGFSLSS | 71 | DIQMTQSPSSLSASVGDRVTITCRAS | 106 |
| 17 | YGVDWVRQAPGKGLEWLGVIWGFGGTN | | QDISNYLNWYQQKPGKAVKLLIYYT | |
| | YNSALKSRLTISRDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLa0015- | EIQLVESGGGLIQPGGSLRISCAVSGFSLSS | 72 | DIQMTQSPSSLSASVGDRVTITCRAS | 106 |
| 18 | YGVDWVRQAPGKGLEWLGVIWGFGGTN | | QDISNYLNWYQQKPGKAVKLLIYYT | |
| | YNSALKSRLTISVDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLa0015- | EIQLVESGGGLIQPGGSLRISCAVSGFSLSS | 73 | DIQMTQSPSSLSASVGDRVTITCRAS | 106 |
| 19 | YGVDWVRQAPGKGLEWLGVIWGFGGTN | | QDISNYLNWYQQKPGKAVKLLIYYT | |
| | YQSALKSRLTISKDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLa0015- | EIQLVESGGGLIQPGGSLRISCAVSGFSLSS | 74 | DIQMTQSPSSLSASVGDRVTITCRAS | 106 |
| 20 | YGVDWVRQAPGKGLEWLGVIWGFGGTN | | QDISNYLNWYQQKPGKAVKLLIYYT | |
| | YNAALKSRLTISKDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLa0015- | EIQLVESGGGLIQPGGSLRISCAVSGFSLSS | 75 | DIQMTQSPSSLSASVGDRVTITCRAS | 106 |
| 21 | YGVDWVRQAPGKGLEWLGVIWGFGGTN | | QDISNYLNWYQQKPGKAVKLLIYYT | |
| | YNSALKSRLTISADNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSY | 76 | DIQMTQSPSSLSASVGDRVTITCRAS | 106 |
| 22 | YGVDWVRQAPGKGLEWLGVIWGFGGTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSS | 77 | DIQMTQSPSSLSASVGDRVTITCRAS | 106 |
| 23 | YGVDWVRQAPGKGLEWLGVIWGFGGTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNYDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSS | 78 | DIQMTQSPSSLSASVGDRVTITCRAS | 106 |
| 24 | YGVDWVRQAPGKGLEWLGVIWGFGGTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGDFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSS | 79 | DIQMTQSPSSLSASVGDRVTITCRAS | 106 |
| 25 | YGVDWVRQAPGKGLEWLGVIWGFGGTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGLFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLST | 80 | DIQMTQSPSSLSASVGDRVTITCRAS | 106 |
| 26 | YGVDWVRQAPGKGLEWLGVIWGFGGTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLIS | 81 | DIQMTQSPSSLSASVGDRVTITCRAS | 106 |
| 27 | YGVDWVRQAPGKGLEWLGVIWGFGGTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSS | 82 | DIQMTQSPSSLSASVGDRVTITCRAS | 106 |
| 28 | YGVDWVRQAPGKGLEWLGVIWGFGGTN | | QDISNYLNWYQQKPGKAVKLLIYYT | 0 |
| | YNPALKSRLTISKDNSKNTVYLQMNSLRA | | SRLHSGVPSRFSGSGSGTDYTLTISSL | 6 |
| | EDTAVYYCASGNFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLKS | 83 | DIQMTQSPSSLSASVGDRVTITCRAS | 106 |
| 29 | YGVDWVRQAPGKGLEWLGVIWGFGGTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSS | 84 | DIQMTQSPSSLSASVGDRVTITCRAS | 106 |
| 30 | YGVDWVRQAPGKGLEWLGVIWGFGGTH | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSS | 85 | DIQMTQSPSSLSASVGDRVTITCRAS | 106 |
| 31 | YGVDWVRQAPGKGLEWLGRIWGFGGTN | | QDISNYLNWYQQKPGKAVKLLIYYT | 0 |
| | YNSALKSRLTISKDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLA015- | EIQLVESGGGLIQPGGSLRISCAVSGFDLSS | 86 | DIQMTQSPSSLSASVGDRVTITCRAS | 106 |
| 32 | YGVDWVRQAPGKGLEWLGVIWGFGGTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSS | 87 | DIQMTQSPSSLSASVGDRVTITCRAS | 106 |
| 33 | YGVDWVRQAPGKGLEWLGVIWGFGKTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSS | 88 | DIQMTQSPSSLSASVGDRVTITCRAS | 106 |
| 34 | YGVDWVRQAPGKGLEWLGVIWGFGGTD | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSY | 89 | DIQMTQSPSSLSASVGDRVTITCRAS | 106 |
| 35 | YGVDWVRQAPGKGLEWLGVIWGFGGTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNYDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSY | 90 | DIQMTQSPSSLSASVGDRVTITCRAS | 106 |
| 36 | YGVDWVRQAPGKGLEWLGVIWGFGGTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGDFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSY | 91 | DIQMTQSPSSLSASVGDRVTITCRAS | 106 |
| 37 | YGVDWVRQAPGKGLEWLGVIWGFGGTN | | QDISNYLNWYQQKPGKAVKLLIYYT | |
| | YNPALKSRLTISKDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSY | 92 | DIQMTQSPSSLSASVGDRVTITCRAS | 106 |
| 38 | YGVDWVRQAPGKGLEWLGVIWGFGGTH | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSY | 93 | DIQMTQSPSSLSASVGDRVTITCRAS | 106 |
| 39 | YGVDWVRQAPGKGLEWLGRIWGFGGTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLA015- | EIQLVESGGGLIQPGGSLRISCAVSGFDLSY | 94 | DIQMTQSPSSLSASVGDRVTITCRAS | 106 |
| 40 | YGVDWVRQAPGKGLEWLGVIWGFGGTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLIS | 95 | DIQMTQSPSSLSASVGDRVTITCRAS | 106 |
| 41 | YGVDWVRQAPGKGLEWLGVIWGFGGTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNYDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSS | 96 | DIQMTQSPSSLSASVGDRVTITCRAS | 106 |
| 42 | YGVDWVRQAPGKGLEWLGVIWGFGGTN | | QDISNYLNWYQQKPGKAVKLLIYYT | |
| | YNPALKSRLTISKDNSKNTVYLQMNSLRA | | SRLHSGVPSRFSGSGSGTDYTLTISSL | |
| | EDTAVYYCASGNYDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSS | 97 | DIQMTQSPSSLSASVGDRVTITCRAS | 106 |
| 43 | YGVDWVRQAPGKGLEWLGVIWGFGGTH | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNYDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSS | 98 | DIQMTQSPSSLSASVGDRVTITCRAS | 106 |
| 44 | YGVDWVRQAPGKGLEWLGRIWGFGGTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNYDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSS | 99 | DIQMTQSPSSLSASVGDRVTITCRAS | 106 |
| 45 | YGVDWVRQAPGKGLEWLGVIWGFGKTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGNYDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLIS | 100 | DIQMTQSPSSLSASVGDRVTITCRAS | 106 |
| 46 | YGVDWVRQAPGKGLEWLGVIWGFGGTN | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGDFDAMDYWGQGTLVTV | | QPEDFATYFCQQGNTLPFTFGQGTKL | |
| | SS | | EIK | |
| TLA015- | EIQLVESGGGLIQPGGSLRISCAVSGFSLSS | 101 | DIQMTQSPSSLSASVGDRVTITCRAS | 106 |
| 47 | YGVDWVRQAPGKGLEWLGVIWGFGGTH | | QDISNYLNWYQQKPGKAVKLLIYYT | |
| | YNSALKSRLTISKDNSKNTVYLQMNSLRA | | SRLHSGVPSRFSGSGSGTDYTLTISSL | |
| | EDTAVYYCASGDFDAMDYWGQGTLVTV | | QPEDFATYFCQQGNTLPFTFGQGTKL | |
| | SS | | EIK | |

where the single underlined bolded amino acids represent the portion of the mutation compared to TLA0015-11.

4.2 TLA0238 humanized design: According to the VH and VL amino acid sequences TLA0238 murine monoclonal antibodies, IGHV3-15/IGHV3-7/IGKV3-11/IGKV1-39 Germline, which has the highest homology in the human germline database, was selected as a template, and the CDR region of the murine antibody was transplanted to the corresponding humanized template to obtain the humanized sequence, and the heavy and light chain pairs were paired to obtain TLA0238 humanized antibody sequence pairs, and some sequences were PTM Removal, the specific sequences are shown in Table 5

TABLE 5

| TLA00238 | | | | |
|---|---|---|---|---|
| humanized | | SEQ | | SEQ |
| antibody | | ID | | ID |
| sequence | VH | NO | VL | NO |
| TLA0238- | EVQVVESGGGLVKPGGSLRLSCAASGF | 203 | ENVLTQSPATLSLSPGERATMSCS | 222 |
| 1 | TFSNYWMNWVRQAPGKGLEWVAQIR | | ASSSVSYMHWYQQKPGQAPRLWI | |
| | LKSDNYATHYAESVKGNFTISRDDSKS | | YDTSNLASGVPARFSGSGSGNDY | |
| | TVYLQMNSLKTEDTAVYYCTPLLLRYR | | TLTISSMEPEDFAVYYCFQENGHP | |
| | DYWGQGTLVTVSS | | FTFGGGTKVEMK | |
| TLA0238- | EVQVVESGGGLVKPGGSLRLSCAASGF | 203 | DNQLTQSPSSLSASVGDRVTITCS | 223 |
| 2 | TFSNYWMNWVRQAPGKGLEWVAQIR | | ASSSVSYMHWYQQKPGKAPKLWI | |
| | LKSDNYATHYAESVKGNFTISRDDSKS | | YDTSNLASGVPGRFSGSGSGNDY | |
| | TVYLQMNSLKTEDTAVYYCTPLLLRYR | | TLTISSLQPEDFATYYCFQENGHPF | |
| | DYWGQGTLVTVSS | | TFGQGTKLEIK | |
| TLA0238- | EVQVVESGGGLVQPGGSLRLSCIASGFT | 204 | ENVLTQSPATLSLSPGERATMSCS | 222 |
| 3 | FSNYWMNWVRQAPGKGLEWVAQIRL | | ASSSVSYMHWYQQKPGQAPRLWI | |
| | KSDNYATHYAESVKGNFTISRDNAKSS | | YDTSNLASGVPARFSGSGSGNDY | |
| | VYLQMNSLRAEDTAVYYCTPLLLRYR | | TLTISSMEPEDFAVYYCFQENGHP | |
| | DYWGQGTLLTVSS | | FTFGGGTKVEMK | |
| TLA0238- | EVQVVESGGGLVQPGGSLRLSCIASGFT | 204 | DNQLTQSPSSLSASVGDRVTITCS | 223 |
| 4 | FSNYWMNWVRQAPGKGLEWVAQIRL | | ASSSVSYMHWYQQKPGKAPKLWI | |
| | KSDNYATHYAESVKGNFTISRDNAKSS | | YDTSNLASGVPGRESGSGSGNDY | |
| | VYLQMNSLRAEDTAVYYCTPLLLRYR | | TLTISSLQPEDFATYYCFQENGHPF | |
| | DYWGQGTLLTVSS | | TFGQGTKLEIK | |
| TLA0238- | EVQVVESGGGLVKPGGSLRLSCAASGF | 205 | DNQLTQSPSSLSASVGDRVTITCS | 223 |
| 2-1 | TFSNYWMNWVRQAPGKGLEWVAQIR | | ASSSVSYMHWYQQKPGKAPKLWI | |
| | LKSDNYATHYAESVKGRFTISRDDSKS | | YDTSNLASGVPGRESGSGSGNDY | |
| | TVYLQMNSLKTEDTAVYYCTPLLLRYR | | TLTISSLQPEDFATYYCFQENGHPF | |
| | DYWGQGTLVTVSS | | TFGQGTKLEIK | |
| TLA0238- | EVQVVESGGGLVKPGGSLRLSCAASGF | 206 | DNQLTQSPSSLSASVGDRVTITCS | 223 |
| 2-2 | TFSNYWMNWVRQAPGKGLEWVAQIR | | ASSSVSYMHWYQQKPGKAPKLWI | |
| | LKSDNYATHYAESVKGNFAISRDDSKS | | YDTSNLASGVPGRFSGSGSGNDY | |
| | TVYLQMNSLKTEDTAVYYCTPLLLRYR | | TLTISSLQPEDFATYYCFQENGHPF | |
| | DYWGQGTLVTVSS | | TFGQGTKLEIK | |

Where the single underline represents the sequence of FR1, FR2, FR3, FR4, and the rest portion is CDR1, CDR2, CDR3 in that order. CDRs are defined by Kabat.

In addition, TLA0238 was humanized and the sequences obtained from the design are shown in Table 6

TABLE 6

| TLA00238 | | | | |
|---|---|---|---|---|
| humanized | | SEQ | | SEQ |
| antibody | | ID | | ID |
| sequence | VH | NO | VL | NO |
| TLA0238-5 | EVQLVESGGGLVQPGGSLRLSCAASGF | 219 | DIQMTQSPSSVSASVGDRVTITCS | 229 |
| | TFSNYWMNWVRQAPGKGLEWVAQIRL | | ASSSVSYMHWYQQKPGKAPKLW | |
| | KSDNYATHYADSVKGRFTISRDNSKNT | | IYDTSNLASGVPSRESGSGSGTDF | |
| | LYLQMNSLRAEDTAVYYCTPLLLRYRD | | TLTISSLQPEDFATYYCFQENGHP | |
| | YWGQGTLVTVSS | | FTFGQGTKVEIK | |
| TLA0238-6 | QVQLVESGGGVVQPGRSLRLSCAASGF | 207 | DIQMTQSPSSVSASVGDRVTITCS | 229 |
| | TFSNYWMNWVRQAPGKGLEWVAQIRL | | ASSSVSYMHWYQQKPGKAPKLW | |
| | KSDNYATHYADSVKGRFTISRDNSKNT | | IYDTSNLASGVPSRESGSGSGTDF | |
| | LYLQMNSLRAEDTAVYYCTPLLLRYRD | | TLTISSLQPEDFATYYCFQENGHP | |
| | YWGQGTMVTVSS | | FTFGQGTKVEIK | |
| TLA0238-7 | EVKVEESGGGLVQPGGSLRLSCIASGFT | 208 | ENVLTQSPAIMSATPGEKVTITCS | 224 |
| | FSNYWMHWVRQAPGKGLEWVGQIRL | | ASSSVSYLHWYQQKPGQSPQLWI | |
| | KSDNYATHYAASVKGRFTISRDNSKNT | | YDTSNRASGVPDRESGSGSGTDY | |
| | LYLQMNSLKTEDTAVYYCTPLLLRYRD | | TLTISSLQPEDVATYYCFQENGHP | |
| | YWGQGTLVTVSS | | FTFGGGTKLEMK | |
| TLA0238-8 | EVKVEESGGGLVQPGGSLKLSCIASGFT | 209 | EVVLTQSPAFLSVTPGEKVTITCS | 225 |
| | FSNYWMHWVRQAPGKGLEWVGQIRL | | ASSSVSYLAWYQQKPGQSPRLWI | |
| | KSDNYATHYAASVKGRFTISRDDSKNS | | YDTSNRASGVPDRESGSGSGTDY | |
| | LYLQMNSLRDEDTAVYYCTPLLLRYRD | | TLTISSLQPEDFAVYYCFQENGHP | |
| | YWGQGTLVTVSS | | FTFGGGTKLEMK | |
| TLA0238-9 | EVKVEESGGGLVQPGGALRLSCIASGFT | 210 | ENVLTQSPAFMSATPGEKVTITCS | 226 |
| | FSNYWMHWVRQAPGKGLEWVGQIRL | | ASSSVSYLAWYQQKPGKSPKLWI | |
| | KSDNYATHYAASVKGRFTISRDDSKNT | | YDTSNLATGVPSRFSGSGSGTSYT | |
| | LYLQMNSLRAEDTAVYYCTPLLLRYRD | | LTISSLQPEDVATYYCFQENGHPF | |
| | YWGQGTLVTVSS | | TFGGGTKLEMK | |
| TLA0238-10 | EVKVEESGGGLVQPGGSLRLSCIASGFT | 211 | EIVLTQSPAFMSATPGEKVTITCS | 227 |
| | FSNYWMHWVRQAPGKGLEWVGQIRL | | ASSSVSYLHWYQQKPDQSPKLWI | |
| | KSDNYATHYAASVKGRFTISRDDSKNS | | YDTSNRASGVPDRESGSGSGTDY | |
| | LYLQMNSLRAEDMAVYYCTPLLLRYR | | TLTISSLEAEDAATYYCFQENGHP | |
| | DYWGQGTLVTVSS | | FTFGGGTKLEMK | |
| TLA0238-11 | EVQLVESGGGLVQPGGSLRLSCAASGF | 212 | DIQMTQSPSSVSASVGDRVTITCS | 229 |
| | TFSNYWMNWVRQAPGKGLEWVAQIRL | | ASSSVSYMHWYQQKPGKAPKLW | |
| | KSDNYATHYADSVKGRFTISRDNSKNT | | IYDTSNLASGVPSRFSGSGSGTDF | |
| | LYLQMNSLRAEDTAVYYCTPLLLRYRD | | TLTISSLQPEDFATYYCFQENGHP | |
| | YWGQGTMVTVSS | | FTFGQGTKVEIK | |
| TLA0238-12 | EVKLEESGGGLVQPGGSLKLSCIASGFT | 213 | ETVLTQSPAFMSATPGEKVTITCS | 228 |
| | FSNYWMHWVRQAPGKGLEWVGQIRL | | ASSSVSYLTWYQQKPGQSPKLWI | |
| | KSDNYATHYAASVKGRFTISRDDSKNT | | YDTSNLASGVPDRESGSGSGTDY | |
| | AYLQMNSLRAEDTAVYYCTPLLLRYR | | TLTISSLEAEDAATYYCFQENGHP | |
| | DYWGQGTLVTVSS | | FTFGGGTKLEMK | |
| TLA0238-13 | EVQLVESGGGLVKPGGSLRLSCAASGF | 214 | DIQMTQSPSSVSASVGDRVTITCS | 229 |
| | TFSNYWMNWVRQAPGKGLEWVAQIRL | | ASSSVSYMHWYQQKPGKAPKLW | |
| | KSDNYATHYAAPVKGRFTISRDDSKNT | | IYDTSNLASGVPSRFSGSGSGTDF | |
| | LYLQMNSLKTEDTAVYYCTPLLLRYRD | | TLTISSLQPEDFATYYCFQENGHP | |
| | YWGQGTTVTVSS | | FTFGQGTKVEIK | |
| TLA0238-14 | EVKLEESGGGLVQPGGALRLSCIASGFT | 215 | ENVLTQSPAFLSVTPGEKVTITCS | 230 |
| | FSNYWMSWVRQAPGKGLEWVGQIRLK | | ASSSVSYLHWYQQKPDQSPKLWI | |
| | SDNYATHYAASVKGRFTISRDDSKNTL | | YDTSNLATGVPSRFSGSGSGTDYT | |
| | YLQMNSLRAEDTAVYYCTPLLLRYRD | | LTISSLQPEDFATYYCFQENGHPF | |
| | YWGQGTLVTVSS | | TFGGGTKLEMK | |
| TLA0238-39 | EVQLVESGGGLVKPGGSLRLSCAASGFT | 332 | EIVLTQSPATLSLSPGERATLSCSAS | 335 |
| | FSNYWMNWVRQAPGKGLEWVGQIRL | | SSVSYMHWYQQKPGQSPRLWIY | |
| | KSDNYATHYAESVKGRFTISRDDSKNTL | | DTSNLASGIPARFSGSGSGTDYTL | |
| | YLQMNSLKTEDTAVYYCTTLLLRYRDY | | TISSLEPEDFAVYYCFQENAHPFTF | |
| | WGQGTLVTVSS | | GGGTKVEIK | |
| TLA0238-40 | EVQLVESGGGLVKPGGSLRLSCAASGFT | 332 | EIVLTQSPATLSLSPGERATLSCSAS | 336 |
| | FSNYWMNWVRQAPGKGLEWVGQIRL | | SSVSYMHWYQQKPGQSPRLWIY | |
| | KSDNYATHYAESVKGRFTISRDDSKNTL | | DTSNLASGVPARFSGSGSGTDYTL | |
| | YLQMNSLKTEDTAVYYCTTLLLRYRDY | | TISSLEPEDFAVYYCFQENAHPFTF | |
| | WGQGTLVTVSS | | GGGTKVEIK | |
| TLA0238-41 | EVQLVESGGGLVKPGGSLRLSCAASGFT | 332 | DIQMTQSPSSLSASVGDRVTITCS | 337 |
| | FSNYWMNWVRQAPGKGLEWVGQIRL | | ASSSVSYMHWYQQKPGKAPKLW | |
| | KSDNYATHYAESVKGRFTISRDDSKNTL | | IYDTSNLASGVPSRFSGSGSGTDY | |
| | YLQMNSLKTEDTAVYYCTTLLLRYRDY | | TLTISSLQPEDFATYYCFQENAHPF | |
| | WGQGTLVTVSS | | TFGQGTKLEIK | |
| TLA0238-42 | EVQLVESGGGLVKPGGSLRLSCAASGFT | 332 | DIQLTQSPSSLSASVGDRVTITCSA | 338 |
| | FSNYWMNWVRQAPGKGLEWVGQIRL | | SSSVSYMHWYQQKPGKAPKLWI | |
| | KSDNYATHYAESVKGRFTISRDDSKNTL | | YDTSNLASGVPSRFSGSGSGTDYT | |
| | YLQMNSLKTEDTAVYYCTTLLLRYRDY | | LTISSLQPEDFATYYCFQENAHPFT | |
| | WGQGTLVTVSS | | FGQGTKLEIK | |
| TLA0238-43 | EVQLVESGGGLVKPGGSLRLSCAASGFT | 333 | EIVLTQSPATLSLSPGERATLSCSAS | 335 |
| | FSNYWMNWVRQAPGKGLEWVGQIRL | | SSVSYMHWYQQKPGQSPRLWIY | |
| | KSDNYATHYAESVKGRFTISRDDSKNTL | | DTSNLASGIPARFSGSGSGTDYTL | |
| | YLQMNSLKTEDTAVYYCTPLLLRYRDY | | TISSLEPEDFAVYYCFQENAHPFTE | |
| | WGQGTLVTVSS | | GGGTKVEIK | |
| TLA0238-44 | EVQLVESGGGLVKPGGSLRLSCAASGFT | 333 | EIVLTQSPATLSLSPGERATLSCSAS | 336 |
| | FSNYWMNWVRQAPGKGLEWVGQIRL | | SSVSYMHWYQQKPGQSPRLWIY | |
| | KSDNYATHYAESVKGRFTISRDDSKNTL | | DTSNLASGVPARFSGSGSGTDYTL | |
| | YLQMNSLKTEDTAVYYCTPLLLRYRDY | | TISSLEPEDFAVYYCFQENAHPFTE | |
| | WGQGTLVTVSS | | GGGTKVEIK | |
| TLA0238-45 | EVQLVESGGGLVKPGGSLRLSCAASGFT | 333 | DIQMTQSPSSLSASVGDRVTITCS | 337 |
| | FSNYWMNWVRQAPGKGLEWVGQIRL | | ASSSVSYMHWYQQKPGKAPKLW | |
| | KSDNYATHYAESVKGRFTISRDDSKNTL | | IYDTSNLASGVPSRFSGSGSGTDY | |
| | YLQMNSLKTEDTAVYYCTPLLLRYRDY | | TLTISSLQPEDFATYYCFQENAHPF | |
| | WGQGTLVTVSS | | TFGQGTKLEIK | |
| TLA0238-46 | EVQLVESGGGLVKPGGSLRLSCAASGFT | 333 | DIQLTQSPSSLSASVGDRVTITCSA | 338 |
| | FSNYWMNWVRQAPGKGLEWVGQIRL | | SSSVSYMHWYQQKPGKAPKLWI | |
| | KSDNYATHYAESVKGRFTISRDDSKNTL | | YDTSNLASGVPSRFSGSGSGTDYT | |
| | YLQMNSLKTEDTAVYYCTPLLLRYRDY | | LTISSLQPEDFATYYCFQENAHPFT | |
| | WGQGTLVTVSS | | FGQGTKLEIK | |
| TLA0238-47 | EVQLVESGGGLVKPGGSLRLSCAASGFT | 334 | EIVLTQSPATLSLSPGERATLSCSAS | 335 |
| | FSNYWMNWVRQAPGKGLEWVAQIRLK | | SSVSYMHWYQQKPGQSPRLWIY | |
| | SDNYATHYAESVKGRFTISRDDSKNTLY | | DTSNLASGIPARFSGSGSGTDYTL | |
| | LQMNSLKTEDTAVYYCTPLLLRYRDYW | | TISSLEPEDFAVYYCFQENAHPFTF | |
| | GQGTLVTVSS | | GGGTKVEIK | |
| TLA0238-48 | EVQLVESGGGLVKPGGSLRLSCAASGFT | 334 | EIVLTQSPATLSLSPGERATLSCSAS | 336 |
| | FSNYWMNWVRQAPGKGLEWVAQIRLK | | SSVSYMHWYQQKPGQSPRLWIY | |
| | SDNYATHYAESVKGRFTISRDDSKNTLY | | DTSNLASGVPARFSGSGSGTDYTL | |
| | LQMNSLKTEDTAVYYCTPLLLRYRDYW | | TISSLEPEDFAVYYCFQENAHPFTF | |
| | GQGTLVTVSS | | GGGTKVEIK | |
| TLA0238-49 | EVQLVESGGGLVKPGGSLRLSCAASGFT | 334 | DIQMTQSPSSLSASVGDRVTITCS | 337 |
| | FSNYWMNWVRQAPGKGLEWVAQIRLK | | ASSSVSYMHWYQQKPGKAPKLW | |
| | SDNYATHYAESVKGRFTISRDDSKNTLY | | IYDTSNLASGVPSRFSGSGSGTDY | |
| | LQMNSLKTEDTAVYYCTPLLLRYRDYW | | TLTISSLQPEDFATYYCFQENAHPF | |
| | GQGTLVTVSS | | TFGQGTKLEIK | |
| TLA0238-50 | EVQLVESGGGLVKPGGSLRLSCAASGFT | 334 | DIQLTQSPSSLSASVGDRVTITCSA | 338 |
| | FSNYWMNWVRQAPGKGLEWVAQIRLK | | SSSVSYMHWYQQKPGKAPKLWI | |
| | SDNYATHYAESVKGRFTISRDDSKNTLY | | YDTSNLASGVPSRFSGSGSGTDYT | |
| | LQMNSLKTEDTAVYYCTPLLLRYRDYW | | LTISSLQPEDFATYYCFQENAHPFT | |
| | GQGTLVTVSS | | FGQGTKLEIK | |

Where the single underline represents the sequence of FR1, FR2, FR3, FR4, and the rest portion is CDR1, CDR2, CDR3 in that order. CDRs are defined by Kabat.

Eleven antibodies were designed by mutating the amino acids of the protein post-translational modification risk sites present in the TLA0238-5 antibody, and the specific antibody sequences are shown in Table 7.

TABLE 7

| | | SEQ | | SEQ |
|---|---|---|---|---|
| | | ID | | ID |
| antibody | VH | NO | VL | NO |
| TLA0238- | EVQLVESGGGLVQPGGSLRLSCAASGFTFS | 216 | DIQMTQSPSSVSASVGDRVTITCSAS | 231 |
| 15 | NYIMNWVRQAPGKGLEWVAQIRLKSDNY | | SSVSYMHWYQQKPGKAPKLWIYDT | |
| | ATHYADSVKGRFTISRDNSKNTLYLQMNS | | SNLASGVPSRFSGSGSGTDFTLTISSL | |
| | LRAEDTAVYYCTPLLLRYRDYWGQGTLVT | | QPEDFATYYCFQESGHPFTFGQGTK | |
| | VSS | | VEIK | |
| TLA0238- | EVQLVESGGGLVQPGGSLRLSCAASGFTFS | 216 | DIQMTQSPSSVSASVGDRVTITCSAS | 232 |
| 16 | NYIMNWVRQAPGKGLEWVAQIRLKSDNY | | SSVSYMHWYQQKPGKAPKLWIYDT | |
| | ATHYADSVKGRFTISRDNSKNTLYLQMNS | | SNLASGVPSRFSGSGSGTDFTLTISSL | |
| | LRAEDTAVYYCTPLLLRYRDYWGQGTLVT | | QPEDFATYYCFQENEHPFTFGQGTK | |
| | VSS | | VEIK | |
| TLA0238- | EVQLVESGGGLVQPGGSLRLSCAASGFTFS | 217 | DIQMTQSPSSVSASVGDRVTITCSAS | 231 |
| 17 | NYLMNWVRQAPGKGLEWVAQIRLKSDNY | | SSVSYMHWYQQKPGKAPKLWIYDT | |
| | ATHYADSVKGRFTISRDNSKNTLYLQMNS | | SNLASGVPSRFSGSGSGTDFTLTISSL | |
| | LRAEDTAVYYCTPLLLRYRDYWGQGTLVT | | QPEDFATYYCFQESGHPFTFGQGTK | |
| | VSS | | VEIK | |
| TLA0238- | EVQLVESGGGLVQPGGSLRLSCAASGFTFS | 217 | DIQMTQSPSSVSASVGDRVTITCSAS | 232 |
| 18 | NYLMNWVRQAPGKGLEWVAQIRLKSDNY | | SSVSYMHWYQQKPGKAPKLWIYDT | |
| | ATHYADSVKGRFTISRDNSKNTLYLQMNS | | SNLASGVPSRFSGSGSGTDFTLTISSL | |
| | LRAEDTAVYYCTPLLLRYRDYWGQGTLVT | | QPEDFATYYCFQENEHPFTFGQGTK | |
| | VSS | | VEIK | |
| TLA0238- | EVQLVESGGGLVQPGGSLRLSCAASGFTFS | 218 | DIQMTQSPSSVSASVGDRVTITCSAS | 231 |
| 19 | NYMMNWVRQAPGKGLEWVAQIRLKSDN | | SSVSYMHWYQQKPGKAPKLWIYDT | |
| | YATHYADSVKGRFTISRDNSKNTLYLQMN | | SNLASGVPSRFSGSGSGTDFTLTISSL | |
| | SLRAEDTAVYYCTPLLLRYRDYWGQGTLV | | QPEDFATYYCFQESGHPFTFGQGTK | |
| | TVSS | | VEIK | |
| TLA0238- | EVQLVESGGGLVQPGGSLRLSCAASGFTFS | 218 | DIQMTQSPSSVSASVGDRVTITCSAS | 232 |
| 20 | NYMMNWVRQAPGKGLEWVAQIRLKSDN | | SSVSYMHWYQQKPGKAPKLWIYDT | |
| | YATHYADSVKGRFTISRDNSKNTLYLQMN | | SNLASGVPSRFSGSGSGTDFTLTISSL | |
| | SLRAEDTAVYYCTPLLLRYRDYWGQGTLV | | QPEDFATYYCFQENEHPFTFGQGTK | |
| | TVSS | | VEIK | |
| TLA0238- | EVQLVESGGGLVQPGGSLRLSCAASGFTFS | 216 | DIQMTQSPSSVSASVGDRVTITCSAS | 233 |
| 21 | NYIMNWVRQAPGKGLEWVAQIRLKSDNY | | SSVSYMHWYQQKPGKAPKLWIYDT | |
| | ATHYADSVKGRFTISRDNSKNTLYLQMNS | | SNLASGVPSRFSGSGSGTDFTLTISSL | |
| | LRAEDTAVYYCTPLLLRYRDYWGQGTLVT | | QPEDFATYYCFQEEGHPFTFGQGTK | |
| | VSS | | VEIK | |
| TLA0238- | EVQLVESGGGLVQPGGSLRLSCAASGFTFS | 216 | DIQMTQSPSSVSASVGDRVTITCSAS | 234 |
| 22 | NYIMNWVRQAPGKGLEWVAQIRLKSDNY | | SSVSYMHWYQQKPGKAPKLWIYDT | |
| | ATHYADSVKGRFTISRDNSKNTLYLQMNS | | SNLASGVPSRFSGSGSGTDFTLTISSL | |
| | LRAEDTAVYYCTPLLLRYRDYWGQGTLVT | | QPEDFATYYCFQENAHPFTFGQGTK | |
| | VSS | | VEIK | |
| TLA0238- | EVQLVESGGGLVQPGGSLRLSCAASGFTFS | 217 | DIQMTQSPSSVSASVGDRVTITCSAS | 233 |
| 23 | NYLMNWVRQAPGKGLEWVAQIRLKSDNY | | SSVSYMHWYQQKPGKAPKLWIYDT | |
| | ATHYADSVKGRFTISRDNSKNTLYLQMNS | | SNLASGVPSRFSGSGSGTDFTLTISSL | |
| | LRAEDTAVYYCTPLLLRYRDYWGQGTLVT | | QPEDFATYYCFQEEGHPFTFGQGTK | |
| | VSS | | VEIK | |
| TLA0238- | EVQLVESGGGLVQPGGSLRLSCAASGFTFS | 217 | DIQMTQSPSSVSASVGDRVTITCSAS | 234 |
| 24 | NYLMNWVRQAPGKGLEWVAQIRLKSDNY | | SSVSYMHWYQQKPGKAPKLWIYDT | |
| | ATHYADSVKGRFTISRDNSKNTLYLQMNS | | SNLASGVPSRFSGSGSGTDFTLTISSL | |
| | LRAEDTAVYYCTPLLLRYRDYWGQGTLVT | | QPEDFATYYCFQENAHPFTFGQGTK | |
| | VSS | | VEIK | |
| TLA0238- | EVQLVESGGGLVQPGGSLRLSCAASGFTFS | 218 | DIQMTQSPSSVSASVGDRVTITCSAS | 233 |
| 25 | NYMMNWVRQAPGKGLEWVAQIRLKSDN | | SSVSYMHWYQQKPGKAPKLWIYDT | |
| | YATHYADSVKGRFTISRDNSKNTLYLQMN | | SNLASGVPSRFSGSGSGTDFTLTISSL | |
| | SLRAEDTAVYYCTPLLLRYRDYWGQGTLV | | QPEDFATYYCFQEEGHPFTFGQGTK | |
| | TVSS | | VEIK | |
| TLA0238- | EVQLVESGGGLVQPGGSLRLSCAASGFTFS | 218 | DIQMTQSPSSVSASVGDRVTITCSAS | 234 |
| 26 | NYMMNWVRQAPGKGLEWVAQIRLKSDN | | SSVSYMHWYQQKPGKAPKLWIYDT | |
| | YATHYADSVKGRFTISRDNSKNTLYLQMN | | SNLASGVPSRFSGSGSGTDFTLTISSL | |
| | SLRAEDTAVYYCTPLLLRYRDYWGQGTLV | | QPEDFATYYCFQENAHPFTFGQGTK | |
| | TVSS | | VEIK | |
| TLA0238- | EVQLVESGGGLVQPGGSLRLSCAASGFTFS | 219 | DIQMTQSPSSVSASVGDRVTITCSAS | 233 |
| 27 | NYWMNWVRQAPGKGLEWVAQIRLKSDN | | SSVSYMHWYQQKPGKAPKLWIYDT | |
| | YATHYADSVKGRFTISRDNSKNTLYLQMN | | SNLASGVPSRFSGSGSGTDFTLTISSL | |
| | SLRAEDTAVYYCTPLLLRYRDYWGQGTLV | | QPEDFATYYCFQEEGHPFTFGQGTK | |
| | TVSS | | VEIK | |
| TLA0238- | EVQLVESGGGLVQPGGSLRLSCAASGFTFS | 219 | DIQMTQSPSSVSASVGDRVTITCSAS | 234 |
| 28 | NYWMNWVRQAPGKGLEWVAQIRLKSDN | | SSVSYMHWYQQKPGKAPKLWIYDT | |
| | YATHYADSVKGRFTISRDNSKNTLYLQMN | | SNLASGVPSRFSGSGSGTDFTLTISSL | |
| | SLRAEDTAVYYCTPLLLRYRDYWGQGTLV | | QPEDFATYYCFQENAHPFTFGQGTK | |
| | TVSS | | VEIK | |
| TLA0238- | EVQLVESGGGLVQPGGSLRLSCAASGFTFS | 220 | DIQMTQSPSSVSASVGDRVTITCSAS | 233 |
| 29 | NYYMNWVRQAPGKGLEWVAQIRLKSDNY | | SSVSYMHWYQQKPGKAPKLWIYDT | |
| | ATHYADSVKGRFTISRDNSKNTLYLQMNS | | SNLASGVPSRFSGSGSGTDFTLTISSL | |
| | LRAEDTAVYYCTPLLLRYRDYWGQGTLVT | | QPEDFATYYCFQEEGHPFTFGQGTK | |
| | VSS | | VEIK | |
| TLA0238- | EVQLVESGGGLVQPGGSLRLSCAASGFTFS | 220 | DIQMTQSPSSVSASVGDRVTITCSAS | 234 |
| 30 | NYYMNWVRQAPGKGLEWVAQIRLKSDNY | | SSVSYMHWYQQKPGKAPKLWIYDT | |
| | ATHYADSVKGRFTISRDNSKNTLYLQMNS | | SNLASGVPSRFSGSGSGTDFTLTISSL | |
| | LRAEDTAVYYCTPLLLRYRDYWGQGTLVT | | QPEDFATYYCFQENAHPFTFGQGTK | |
| | VSS | | VEIK | |

where the single underlined bolded amino acids represent the portion of the mutation compared to TLA0238-5.

4.3 TLA0241 humanized design: humanizing the TLA0241, and the sequences obtained by the design are shown in Table 8.

TABLE 8

| TLA0241 | | SEQ | | SEQ |
|---|---|---|---|---|
| humanized | | ID | | ID |
| number | VH | NO | VL | NO |
| TLA0241-1 | EVQLVESGGGLVQPGGSLRLSCSASGYTF | 298 | DIQMTQSPSSLSASVGDRVTITCST | 309 |
| | TDYYLNWVRQAPGKGLEWIGDINPNNG | | SSSIISNYSHWYQQKPGKAPKLLIY | |
| | RTTYADSVKGRFTISRDNSKNTLYLQMSS | | KTSNLPSGVPSRFSGSGSGTEFTLTI | |
| | LRAEDTAVYYCAREGGYGWYFDVWGR | | SSLQPEDFATYYCQQGSDMPITFG | |
| | GTLVTVSS | | QGTKVEIK | |
| TLA0241-2 | QVQLVESGGGVVQPGRSLRLSCAASGYT | 299 | DIQMTQSPSSVSASVGDRVTITCST | 315 |
| | FTDYYLNWVRQAPGKGLEWIGDINPNNG | | SSSIISNYSHWYQQKPGKAPKLLIY | |
| | RTTYADSVKGRFTISRDNSKNTLYLQMN | | KTSNLPSGVPSRESGSGSGTDFTLTI | |
| | SLRAEDTAVYYCAREGGYGWYFDVWG | | SSLQPEDFATYYCQQGSDMPITFG | |
| | QGTMVTVSS | | QGTKVEIK | |
| TLA0241-3 | EVQLQQSGPEVKKPGASVRISCKASGYTF | 300 | EIVLTQSPTTLSLSPGEKVTITCSAS | 310 |
| | TDYYMHWVRQAPGQGLEWIGDINPNNG | | SSIISNYLHWYQQKPGKSPKLLIYK | |
| | RTTYNQKFQGRVTITRDTSSSTAYMELRS | | TSNLPSGVPSRESGSGSGTDFTLTIN | |
| | LRSDDTAVYYCAREGGYGWYFDVWGT | | SLEAEDAATYYCQQGSDMPITFGA | |
| | GTTVTVSS | | GTRLELK | |
| TLA0241-4 | EVQLQQSGPEVKKPGASVRISCKASGYTF | 301 | EIVLTQSPPTLSLSPGEKVTITCSAS | 311 |
| | TDYYMHWVQQAPGKGLEWIGDINPNNG | | SSIISNYLHWYQQKPGQSPQLLIYK | |
| | RTTYNQKFQGRVTITADKSTSTAYMELR | | TSNRPSGVPDRFSGSGSGTDFTLTI | |
| | SLRSDDTAVYYCAREGGYGWYFDVWGT | | NSLEAEDAATYYCQQGSDMPITFG | |
| | GTLVTVSS | | AGTRLELK | |
| TLA0241-5 | ELQLQQSGPEVKKPGASVRISCKASGYTE | 302 | EIVLTQSPGTLSLSPGEKVTITCSAS | 312 |
| | TDYYMHWVQQAPGKGLEWIGDINPNNG | | SSIISNYLHWYQQKPDQSPKLLIYK | |
| | RTTYNQKFQGRVTITADKSTSTAYMELR | | TSNLPSGVPSRFSGSGSGTDFTLTIN | |
| | SLTSDDTAVYYCAREGGYGWYFDVWGT | | SLEAEDAATYYCQQGSDMPITFGA | |
| | GTTVTVSS | | GTKLELK | |
| TLA0241-6 | EVQLLESGGGLVQPGGSLRLSCAASGYTF | 303 | AIQLTQSPSSLSASVGDRVTITCSTS | 313 |
| | TDYYLNWVRQAPGKGLEWIGDINPNNG | | SSIISNYSHWYQQKPGKAPKLLIYK | |
| | RTTYADSVKGRFTISRDNSKNTLYLQMN | | TSNLPSGVPSRFSGSGSGTDFTLTIS | |
| | SLRAEDTAVYYCAREGGYGWYFDVWG | | SLQPEDFATYYCQQGSDMPITFGQ | |
| | QGTMVTVSS | | GTKVEIK | |
| TLA0241-7 | EVQLQQSGPEVKKPGASVRISCKASGYTE | 304 | EIVLTQSPATLSLSPGEKVTITCSAS | 314 |
| | TDYYMHWVRQAPGEGLEWIGDINPNNG | | SSIISNYSAWYQQKPGKSPKLLIYK | |
| | RTTYNQKFQGRVTITADKSTSTAYMELR | | TSNLPSGVPARFSGSGSGTDFTLTI | |
| | SLRSDDTAVYYCAREGGYGWYFDVWGT | | NSLEAEDAATYYCQQGSDMPITFG | |
| | GTTVTVSS | | AGTRLELK | |
| TLA0241-8 | QVQLVESGGGVVQPGRSLRLSCAASGYT | 305 | DIQMTQSPSSVSASVGDRVTITCST | 315 |
| | FTDYYLNWVRQAPGKGLEWIGDINPNNG | | SSSIISNYSHWYQQKPGKAPKLLIY | |
| | RTTYADSVKGRFTISRDNSKNTLYLQMN | | KTSNLPSGVPSRFSGSGSGTDFTLTI | |
| | SLRAEDTAVYYCAREGGYGWYFDVWG | | SSLQPEDFATYYCQQGSDMPITFG | |
| | QGTTVTVSS | | QGTKVEIK | |
| TLA0241-9 | EIQLQQSGAEEKKPGASVRISCKASGYTF | 306 | EIVLTQSPTTLSLSPGEKVTITCSAS | 316 |
| | TDYYMHWVQQAPGKGLEWIGDINPNNG | | SSIISNYLHWYQQKPDQSPKLLIYK | |
| | RTTYNQKFQGRVTITADKSTSTAYMELR | | TSNLPSGVPSRFSGSGSGTDFTLTIN | |
| | SLRSDDTAVYYCAREGGYGWYFDVWGT | | SLEAEDAATYYCQQGSDMPITFGA | |
| | GTTVTVSS | | GTKLELK | |
| TLA0241-10 | ELQLQQSGAEVKKPGASVRISCKASGYTF | 307 | EIVLTQSPPTLSLSPGEKVTITCSAS | 317 |
| | TDYYMHWVRQAPGQGLEWIGDINPNNG | | SSIISNYLAWYQQKPGKSPKLLIYK | |
| | RTTYNQKFQGRVTITADKSTSTAYMELR | | TSNLPSGVPSRFSGSGSGTSFTLTIN | |
| | SLRSDDMAVYYCAREGGYGWYFDVWG | | SLEAEDAATYYCQQGSDMPITFGA | |
| | TGTLVTVSS | | GTKLELK | |

The single underline represents the sequence of FR1, FR2, FR3, FR4, and the rest portion is CDR1, CDR2, CDR3 in that order. CDRs are defined by Kabat.

The VH and VL amino acid sequences of the control antibodies RVT-3101 and MK-7240 were obtained according to their published patents, and the specific amino acid sequences are shown in Table 9.

TABLE 9

| Abs | amino acid sequence (VH) | amino acid sequence (VL) |
|---|---|---|
| RVT- | QVQLVQSGAEVKKPGASVKVSCKASGYDF | EIVLTQSPATLSLSPGERATLSCRASQSVSSYLAW |
| 3101 | TYYGISWVRQAPGQGLEWMGWISTYNGNT | YQQKPGQAPRLLIYDASNRATGIPARFSGSGSGT |
| | HYARMLQGRVTMTTDTSTRTAYMELRSLR | DFTLTISSLEPEDFAVYYCQQRSNWPWTFGQGTK |
| | SDDTAVYYCARENYYGSGAYRGGMDVWGQ | VEIK (SEQ ID NO: 323) |
| | GTTVTVSS (SEQ ID NO: 322) | |
| MK- | QVQLVQSGAEVKKPGASVKVSCKASGFDI | EIVLTQSPGTLSLSPGERATLSCRASSSVSYMYW |
| 7240 | QDTYMHWVKQRPGQGLEWMGRIDPASGHT | YQQKPGQAPRPLIYATSNLASGIPDRFSGSGSGTD |
| | KYDPKFQVRVTITRDTSTSTVYLELSSLR | FTLTISRLEPEDFAVYYCQQWEGNPRTFGGGTKL |
| | SEDTAVYYCARSGGLPDVWGQGTTVTVSS | EIK (SEQ ID NO: 325) |
| | (SEQ ID NO: 324) | |

### Example 5 Antibody-Antigen Complex Structure Prediction of Anti-TL1A Monoclonal Antibody

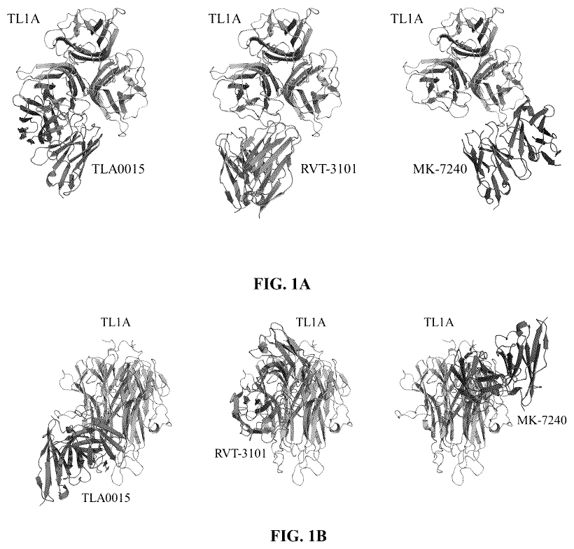

The anti-antibody complex structure prediction model was used to predict the crystal structure of TL1A monomer and the anti-TL1A TLA0015, MK-7240 and RVT-3101, and the high-quality complex structure was obtained. The results of the composite structure prediction are shown in FIGS. 1A and 1B.

### Example 6 Recombinant Antibody Expression and Purification

The VH and VL sequences of the antibodies obtained by sequencing after Single B sorting and phage library panning were constructed as human IgG1 type, with Kappa as the light chain. The amino acid sequences of the Cκ, CH1 and Fc constant region (containing L234A, L235A, M252Y, S254T and T256E mutations) for constructing the antibody are shown in Table 10. The antibody heavy chain and light chain DNA fragments were subcloned into pcDNA3.4 vector, the recombinant plasmid was extracted and co-transfected into CHO cells, and after 7 days of cell culture, the culture medium was filtered through high-speed centrifugation and microporous filter membrane, and then loaded onto a Protein A affinity chromatography column, the protein was eluted with sodium acetate buffer eluent at pH 3.4, and dialyzed to PBS at pH 7.4, and the absorbance value of 280 nm was read by using a NanoDrop instrument to detect the protein concentration. The antibody obtained in this example are used in the tests of the following examples.

TABLE 10

| | |
|---|---|
| Cκ | RTVAAPSVFIFPPSDEQLKSGTASVVCLLNNFYPREAKVQ |
| | WKVDNALQSGNSQESVTEQDSKDSTYSLSSTLTLSKADYE |
| | KHKVYACEVTHQGLSSPVTKSFNRGEC |
| | (SEQ ID NO 318) |
| CH1 | ASTKGPSVFPLAPSSKSTSGGTAALGCLVKDYFPEPVTVS |
| | WNSGALTSGVHTFPAVLQSSGLYSLSSVVTVPSSSLGTQT |
| | YICNVNHKPSNTKVDKKVEPKSC (SEQ ID NO 319) |
| Fc | DKTHTCPPCPAPELLGGPSVFLFPPKPKDTLMISRTPEVT |
| (No | CVVVDVSHEDPEVKFNWYVDGVEVHNAKTKPREEQYNSTY |
| mutation) | RVVSVLTVLHQDWLNGKEYKCKVSNKALPAPIEKTISKAK |
| | GQPREPQVYTLPPSRDELTKNQVSLTCLVKGFYPSDIAVE |
| | WESNGQPENNYKTTPPVLDSDGSFFLYSKLTVDKSRWQQG |
| | NVFSCSVMHEALHNHYTQKSLSLSPGK |
| | (SEQ ID NO 341) |
| Fc | DKTHTCPPCPAPEAAGGPSVFLFPPKPKDTLMISRTPEVT |
| (L234A, | CVVVDVSHEDPEVKFNWYVDGVEVHNAKTKPREEQYNSTY |
| L235A) | RVVSVLTVLHQDWLNGKEYKCKVSNKALPAPIEKTISKAK |
| | GQPREPQVYTLPPSRDELTKNQVSLTCLVKGFYPSDIAVE |
| | WESNGQPENNYKTTPPVLDSDGSFFLYSKLTVDKSRWQQG |
| | NVFSCSVMHEALHNHYTQKSLSLSPGK |
| | (SEQ ID NO 320) |
| Fc | DKTHTCPPCPAPEAAGGPSVFLFPPKPKDTLYITREPEVT |
| (L234A, | CVVVDVSHEDPEVKFNWYVDGVEVHNAKTKPREEQYNSTY |
| L235A, | RVVSVLTVLHQDWLNGKEYKCKVSNKALPAPIEKTISKAK |
| M252Y, | GQPREPQVYTLPPSRDELTKNQVSLTCLVKGFYPSDIAVE |
| S254T, | WESNGQPENNYKTTPPVLDSDGSFFLYSKLTVDKSRWQQG |
| T256E) | NVFSCSVMHEALHNHYTQKSLSLSPGK |
| | (SEQ ID NO 321) |

### Example 7 Biacore Detects the Affinity of the Antibody to the TL1A-his Protein

The affinity of the multiple antibodies prepared by example 6 with human TL1A-his monomeric protein, human TL1A-his trimeric protein, monkey TL1A-his protein and rat TL1A-his protein is detected based on a Biacore biomolecular interaction instrument.

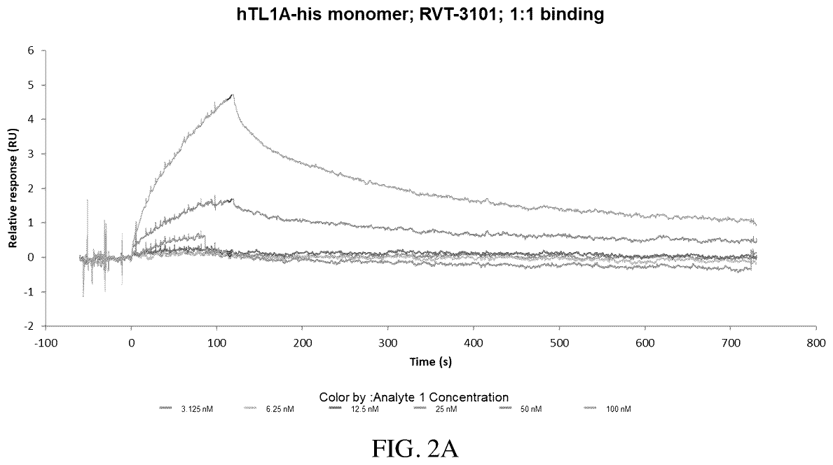

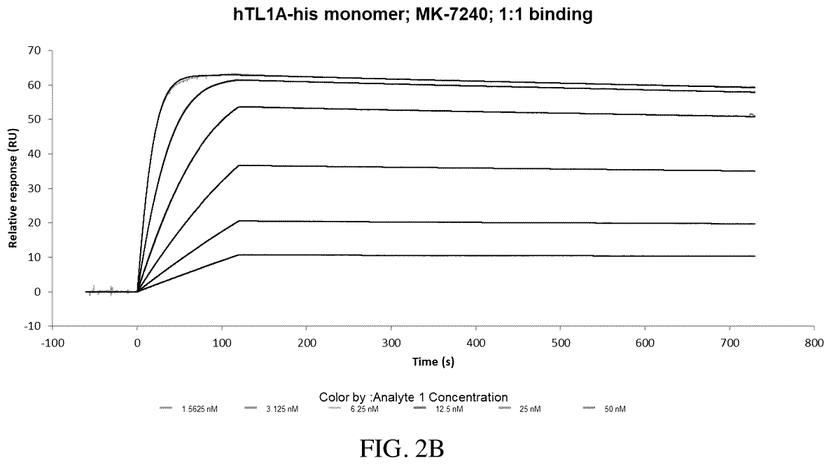

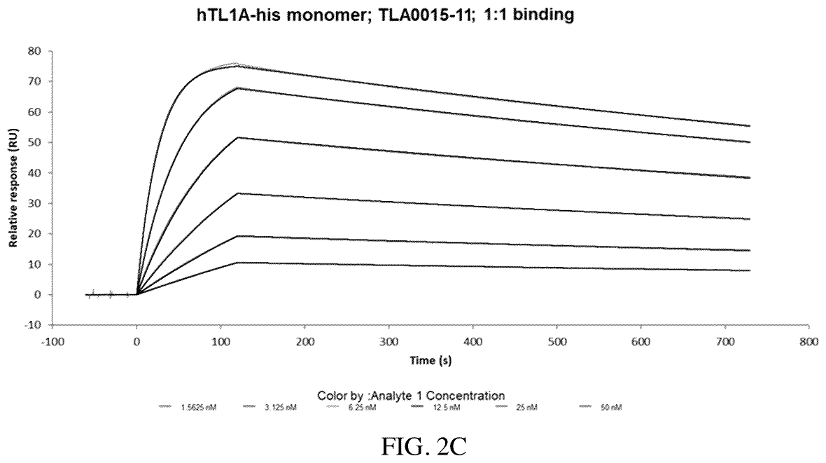

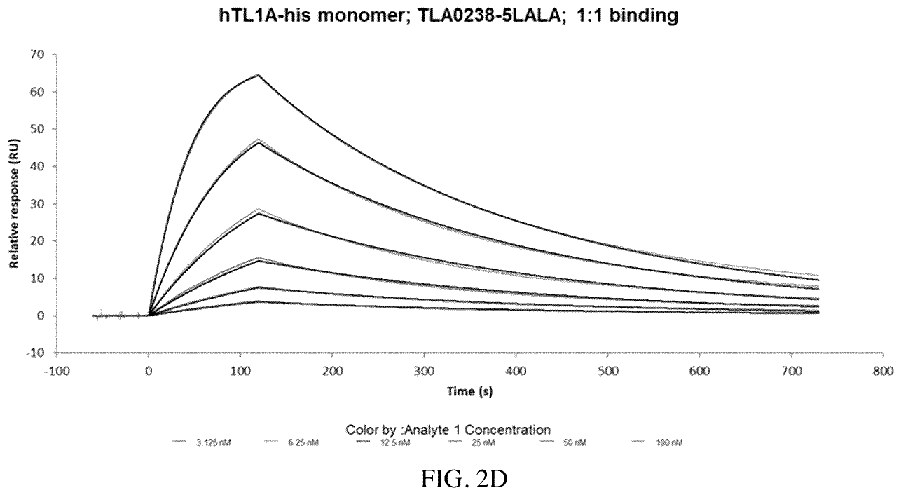

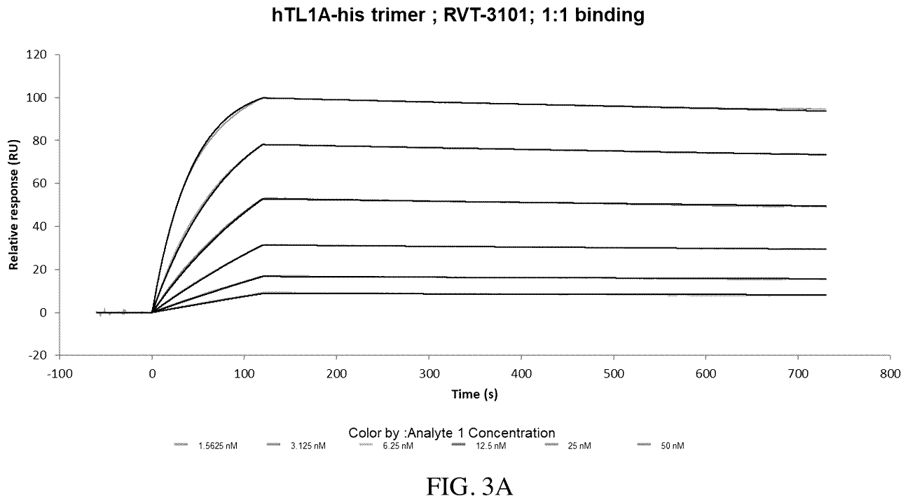

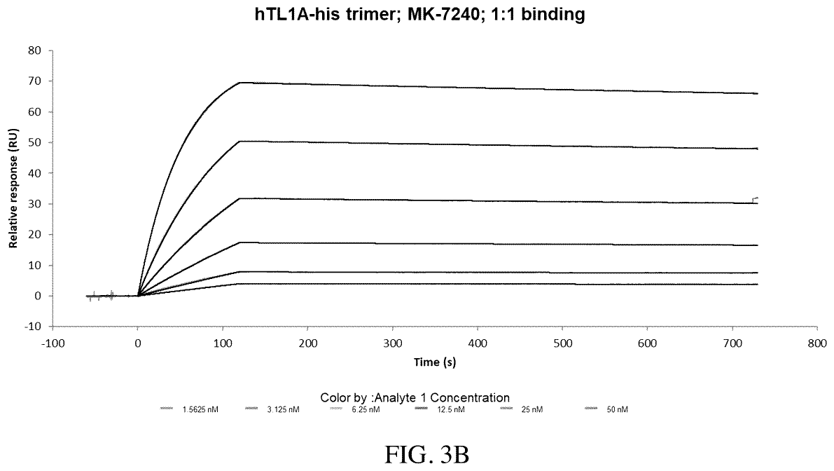

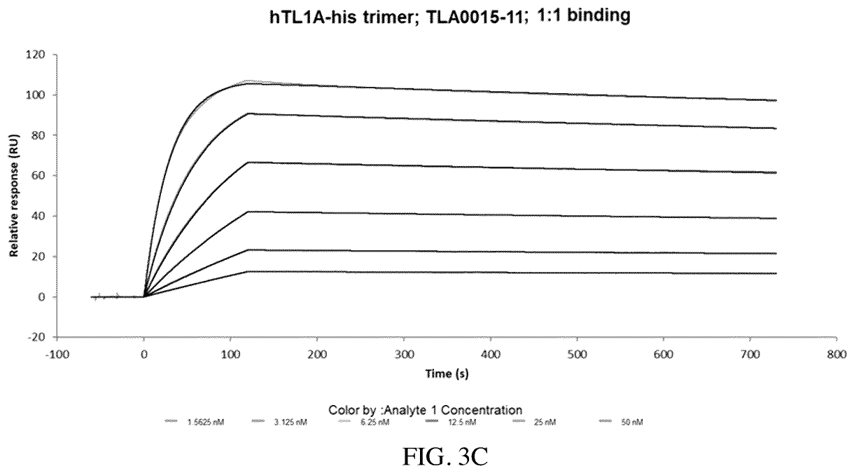

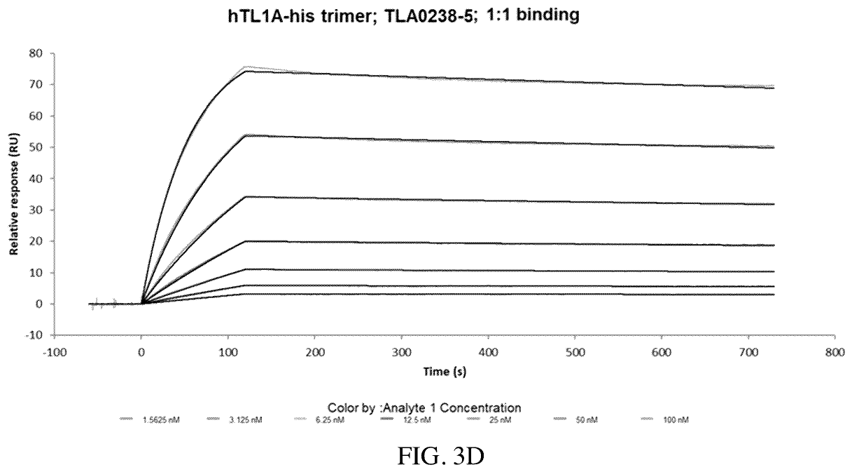

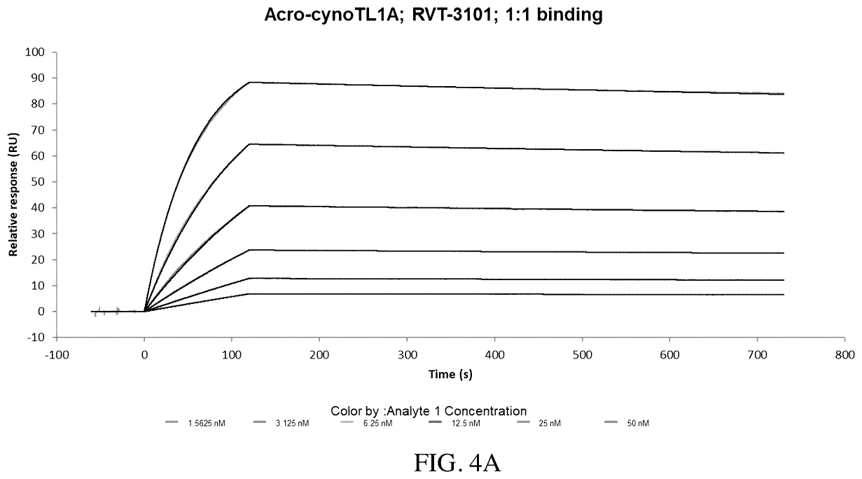

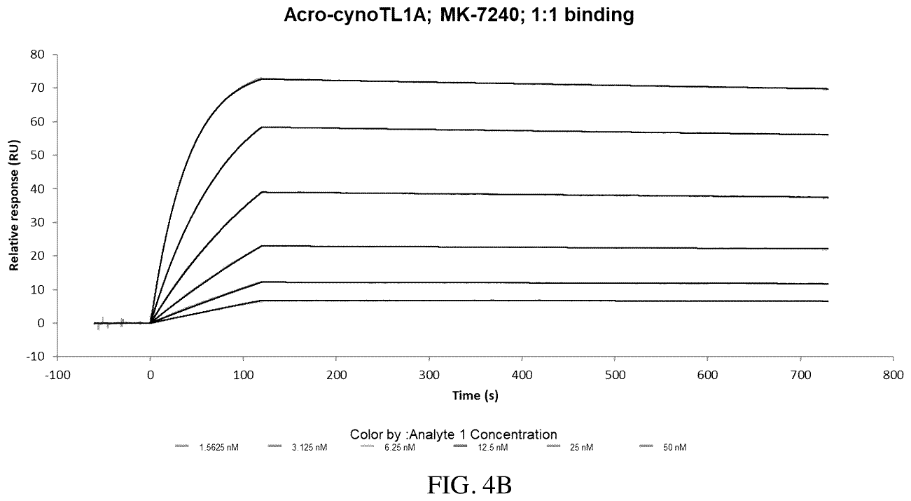

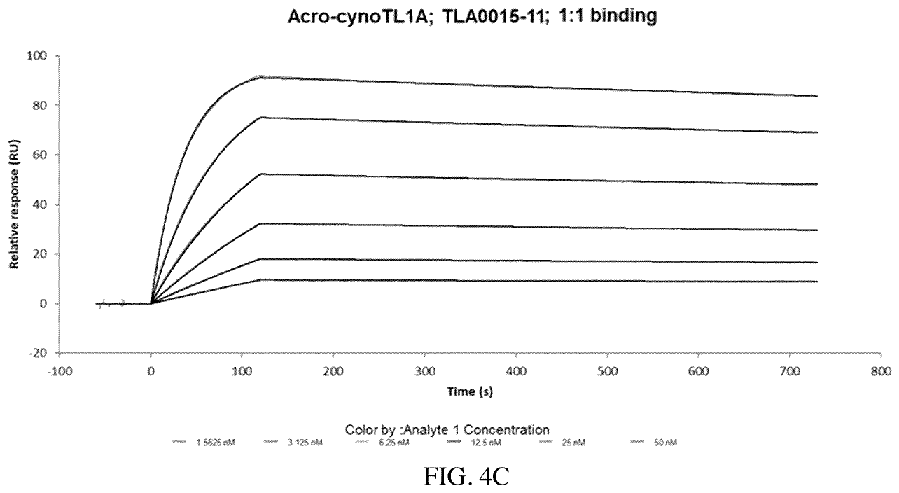

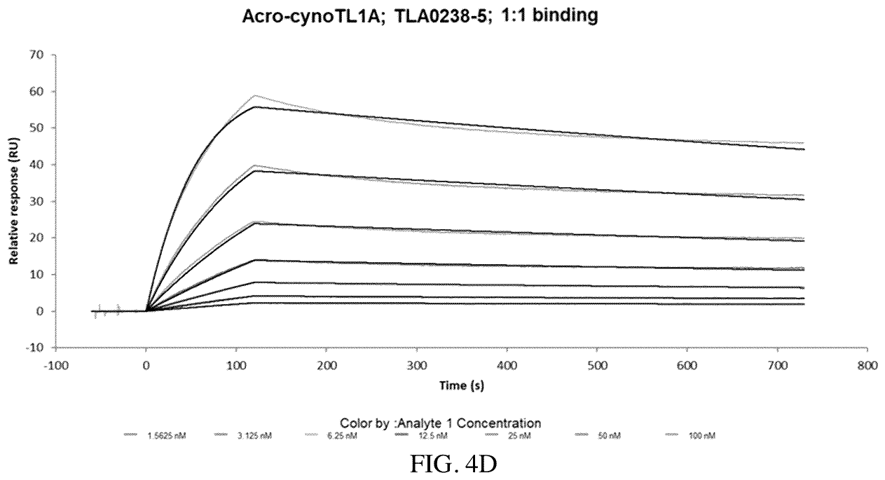

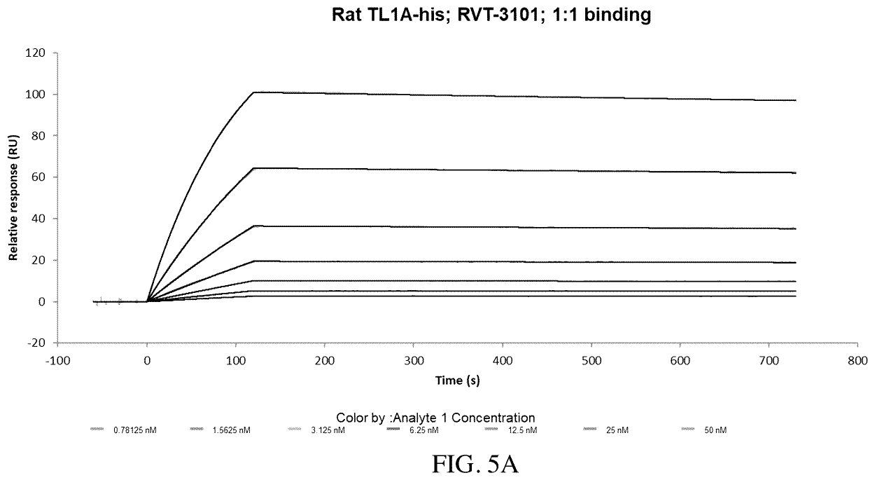

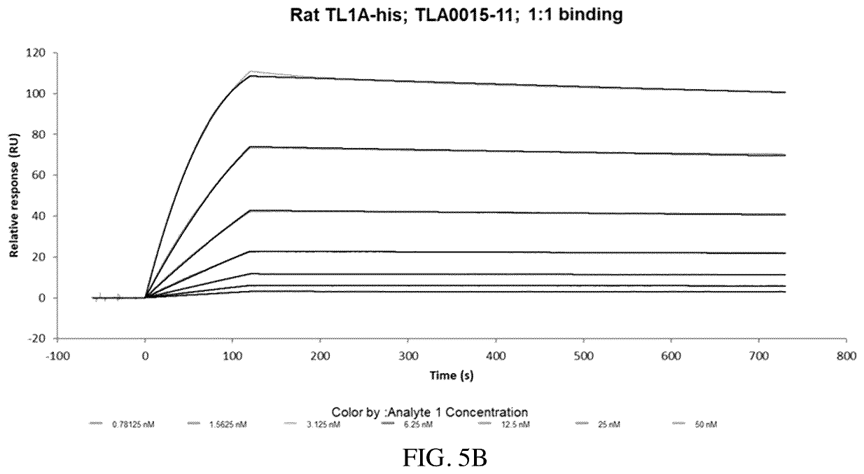

A certain amount of antibody to be tested was captured with the biosensor chip Protein A (GE, 29127556) affinity, and then a series of antigens (50 nM, 25 nM, 12.5 nM, 6.25 nM, 3.125 nM, 1.5625 nM) were passed on the surface of the chip to detect the reaction signal in real time to obtain binding and dissociation curves. After the completion of each cycle of dissociation, the biochips are washed and regenerated with 10 mM glycine-hydrochloric acid solution pH 1.5 (GE, BR-1003-54). The experimental data were fitted to a 1:1 model using Biacore Insight Evaluation Software 5.0 to calculate the value of the affinity. The binding curve of anti-TL1A antibody to human TL1A monomeric protein is shown in FIG. 2. The binding curve of the anti-TL1A antibody to the human TL1A trimeric protein is shown in FIG. 3: The binding curves of anti-TL1A antibody to monkey TL1A protein are shown in FIG. 4. The binding curves of anti-TL1A antibody to rat TL1A protein are shown in FIG. 5, and the affinity KD values are shown in Table 11.

TABLE 11

| Abs | Antigen | ka (1/Ms) | kd (1/s) | KD (M) |
|---|---|---|---|---|
| RVT-3101 | hTL1A-his | No binding | | |
| (Analogue) | monomer | | | |
| MK-7240 | | 1.83e+06 | 1.00e−04 | 5.47e−11 |
| (Analogue) | | | | |
| TLA0015-11 | | 8.39e+05 | 5.05e−04 | 6.02e−10 |
| TLA0238-5 | | 2.66e+05 | 3.91e−03 | 1.47e−08 |
| RVT-3101 | hTL1A-his | 4.70e+05 | 1.04e−04 | 2.21e−10 |
| (Analogue) | trimer | | | |
| MK-7240 | | 3.93e+05 | 8.50e−05 | 2.16e−10 |
| (Analogue) | | | | |
| TLA0015-11 | | 6.84e+05 | 1.36e−04 | 1.99e−10 |
| TLA0238-5 | | 1.86e+05 | 1.23e−04 | 6.57e−10 |
| RVT-3101 | cynoTL1A-his | 3.53e+05 | 8.79e−05 | 2.49e−10 |
| (Analogue) | | | | |
| MK-7240 | | 5.38e+05 | 6.73e−05 | 1.25e−10 |
| (Analogue) | | | | |
| TLA0015-11 | | 5.54e+05 | 1.40e−04 | 2.53e−10 |
| TLA0238-5 | | 2.10e+05 | 3.97e−04 | 1.89e−09 |
| RVT-3101 | Rat TL1A-his | 2.42e+05 | 6.87e−05 | 2.84e−10 |
| (Analogue) | | | | |
| TLA0015-11 | | 5.54e+05 | 1.43e−04 | 2.58e−10 |

### Example 8 ELISA Detects the Binding Ability of Anti-TL1A Antibody to TL1A-his Protein

The binding ability of a plurality of antibodies prepared by example 6 of the present invention to human TL1A-his trimeric protein, mouse TL1A-his protein, monkey TL1A-his protein and rat TL1A-his protein is detected based on ELISA assay. The binding capacity of different antibodies is determined by comparing their binding curves to TL1A-his protein.

Recombinant human TL1A-his trimeric protein, murine TL1A-his, monkey TL1A-his, and rat TL1A-his protein was diluted to 2 μg/ml in PBS at 100 μl/well, respectively, added to the plate (coated with an equal amount of BSA as a control), and incubated overnight at 4° C. Remove the coating solution and incubate at 200 μl/well with 5% BSA blocking solution for 2 h at room temperature. Remove the blocking solution, wash three times with 250 μl/well 0.5% % PBST, then dilute the anti-TL1A specific antibody to 100 nM with the blocking solution, dilute 10-fold to form 8 concentration gradients (the highest concentration is 100 nM), add 100 μl/well sequentially to the blocked microplate plate, and incubate for 1 hour at room temperature. Wash the plate 3 times with PBST (remove residual droplets with absorbent paper) at 100 μl/well, add goat anti-human IgG antibody containing HRP label, and incubate for 45 min at room temperature. Wash the plate 5 times with 0.5‰ PBST at 100 μl/well, add TMB solution, incubate for 5 min at room temperature in the dark, at 100 μl/well, add stop solution, stop the substrate chromogenic reaction, read the OD value at 450 nm with a microplate reader, analyze the data with a GraphPad and calculate the EC50 (Table 12).

TABLE 12
EC50 values of anti-TL1A monoclonal antibody binding
to TL1A protein ELISA in different species

| | ELISA Binding EC50(nM) | | | |
|---|---|---|---|---|
| | huTL1A- | Mouse | Cyno | Rat |
| Abs | his | TL1A-his | TL1A-his | TL1A-his |
| RVT-3101 | 0.04 | 0.04 | 0.05 | 0.03 |
| (Analogue) | | | | |
| MK-7240 | 0.06 | NA | 0.065 | NA |
| (Analogue) | | | | |
| TLA0015 | 0.04 | 0.31 | 0.05 | 0.03 |
| TLA0118 | 0.04 | NA | 0.05 | NA |
| TLA0238 | 0.03 | | 0.04 | |
| TLA0140 | 0.03 | | 0.05 | |
| TLA0148 | 0.04 | | 0.05 | |
| TLA0162 | 0.05 | | 0.06 | |
| TLA0165 | 0.04 | | 0.05 | |
| TLA0167 | 0.04 | | 0.05 | |

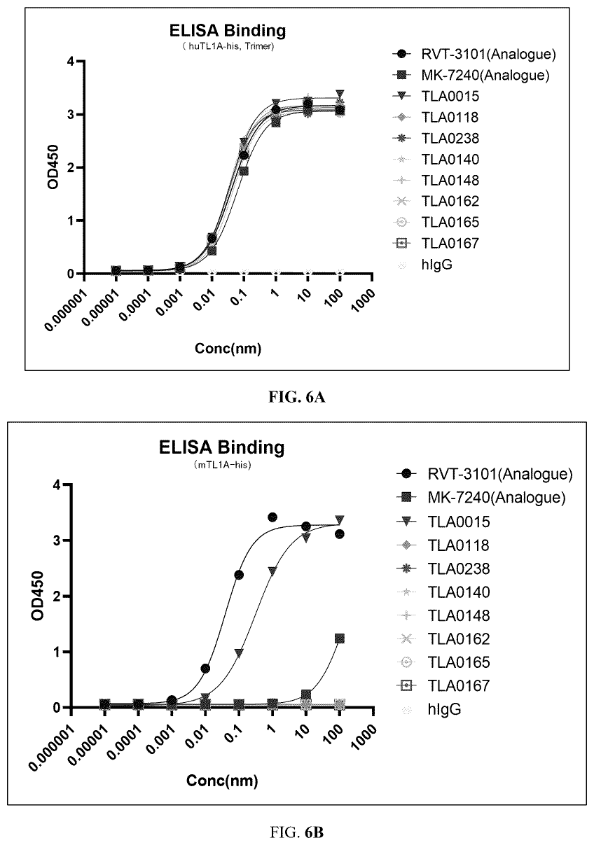

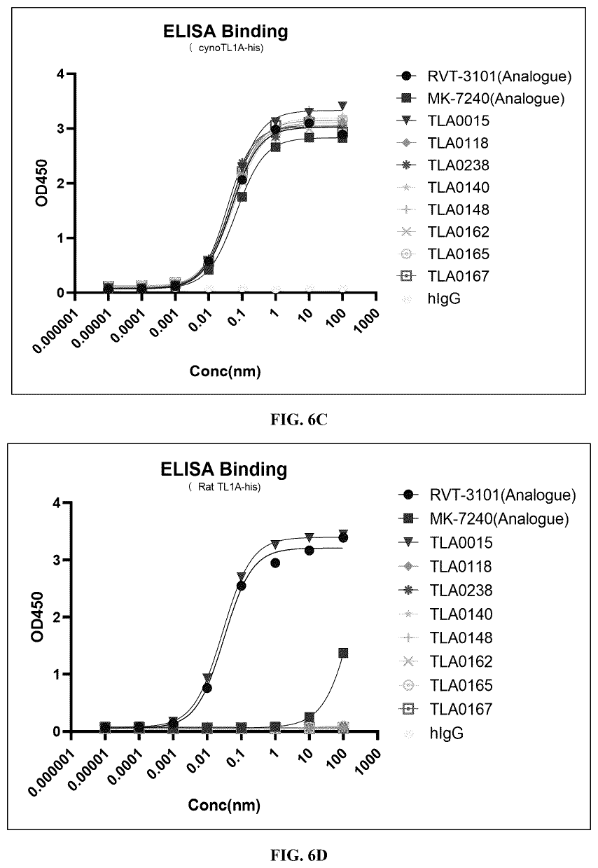

The results of the assay are shown in FIG. 6, TLA0015, TLA0118, TLA0140, TLA0148, TLA0162, TLA00165, TLA0167, TLA0238 showed good binding activity with both human TL1A trimer protein and monkey TL1A protein, among which TLA0015 showed good binding activity with mouse TL1A protein and rat TL1A protein. TLA0015 also showed good binding activity to rat TL1A protein.

### Example 9 FACS Detects the Binding Ability of Anti-TL1A Antibody to 293-TL1A Cell Line

Based on flow cytometry assay, the binding ability of a plurality of antibodies prepared by example 6 to human TL1A molecules expressed on the surface of CHO-K1 cells was detected. The binding capacity of different antibodies was determined by comparing the binding curves of different antibodies to human TL1A molecules expressed on the surface of CHO-K1 cells.

The DNA sequence encoding the full-length protein of human TL1A was cloned into mDeZ (Zeocin resistance gene) vector, transiently transfected into CHO-K1 cells, and screened for 7 days by adding 100 μg/mL Zeocin (final concentration) after 24 h. After subcloning, a CHO-K1 cell line with high expression of human TL1A on the cell surface (CHO-K1-huTL1A) was obtained.

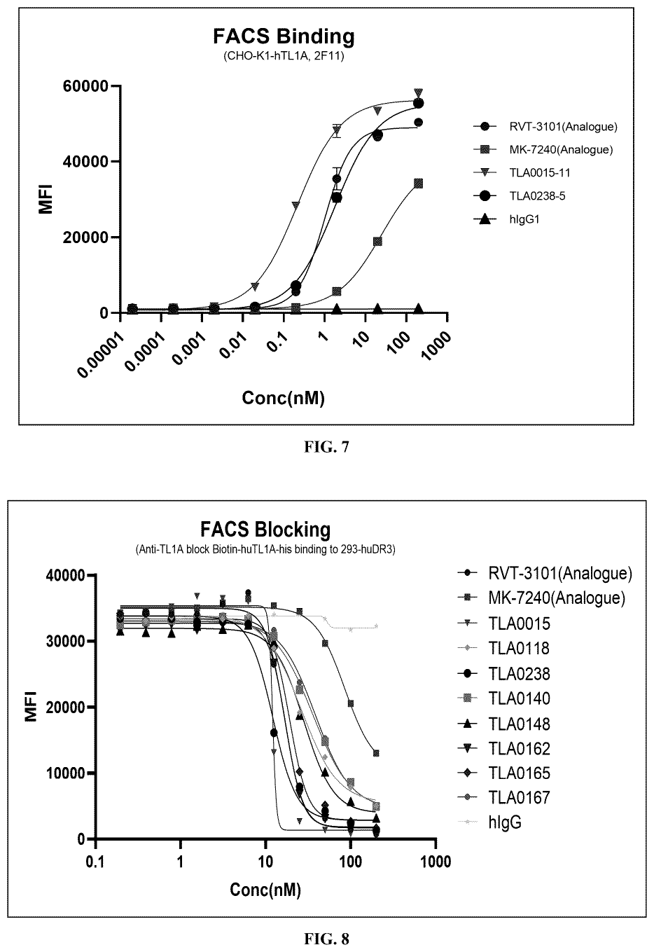

In this experiment, CHO-K1 cells (CHO-K1-huTL1A) with high expression of human TL1A full-length protein on the cell surface were used as the target cells, and CHO-K1 cells were used as the negative control. CHO-K1-huTL1A and CHO-K1 cells were washed three times with staining buffer (PBS+2% FBS (Gibco, Cat #10091148)+5 mM EDTA (Gibco, Cat #15575020)) at 450 g each for 5 min and the supernatant was discarded. Resuspend cells in staining buffer and adjust the cell density to 1×10⁶ cell/mL, 100 μL/well, and add to a 96-well plate. Anti-TL1A antibody and positive control antibody were diluted to 200 nM, 10-fold stepwise dilution of 8 gradients (maximum concentration 200 nM), 100 μL/well, added to a 96-well plate, and mixed well with CHO-K1-huTL1A cells. After 30 min of incubation at 4° C., the cells are washed twice in PBS to remove unbound antibodies to be tested. Add 100 μL/well of goat anti-human IgG-PE and incubate at 4° C. for 30 min. Centrifuge at 300 g for 5 min and wash the cells twice in PBS to remove unbound secondary antibody. Finally, the cells were resuspended in 200 μl PBS, and the binding capacity of the anti-TL1A antibody to CHO-K1-huTL1A cells was determined by a Beckman Coulter CytoFLEX flow cytometer. The resulting data were fitted and analyzed by GraphPad Prism software. The experimental results are shown in FIG. 7 and Table 13, and TLA0015-11 and TLA0238-5 have strong binding capacity to the CHO-K1-huTL1A cell line.

TABLE 13
EC50 of anti-TL1A antibody binding to CHO-K1-huTL1A cells

| | | Top |
|---|---|---|
| Abs | EC50(nM) | MFI |
| RVT-3101 | 1.02 | 49017 |
| (Analogue) | | |
| MK-7240 | 24.95 | 40329 |
| (Analogue) | | |
| TLA0015-11 | 0.41 | 56310 |
| TLA0238-5 | 1.81 | 55419 |

### Example 10 FACS Detects the Ability of TL1A Antibody to Block the Binding of TL1A-his to 293-DR3 Cells

The ability of multiple antibodies prepared by example 6 to block the binding of huTL1A-his protein to human DR3 (DR3: UniprotKB NO. Q93038) expressed on the surface of Expi293 cells was detected based on a flow cytometry assay. The blocking ability of huTL1A-his protein is determined by comparing the curves of different antibody concentrations blocking huTL1A-his protein to 293-huDR3 cells.

The DNA sequence of the receptor DR3 extracellular domain protein encoding TL1A was cloned into the mDeZ-TM (with transmembrane sequence and Zeocin resistance gene) vector, transiently transfected into Expi293 cells, and 100 μg/mL Zeocin (final concentration) was added 24 h later for 7 days to obtain the Expi293 cell line (293-huDR3) expressing human DR3 on the cell surface. The huTL1A-his-Biotin protein was obtained by Biotin conjugation using a biotin labeling kit (ThermoFisher, A35389). FACS Binding was used to determine the EC80 concentration of protein binding between huTL1A-his-Biotin protein and 293-huDR3 cells. Wash 293-huDR3 cells three times with staining buffer (PBS+2% FBS (Gibco, Cat #10091148)+5 mM EDTA (Gibco, Cat #15575020)) at 300 g each for 5 min and discard the supernatant. Resuspend cells in PBS, adjust the cell density to 5×10⁵ cells/mL, 100 μL/well, add to a 96-well plate, centrifuge to remove the supernatant, and add 2*EC80 concentration of huTL1A-his-Biotin protein, 50 μL/well. Anti-TL1A antibody and positive control antibody were diluted to 400 nM, 2-fold stepwise dilution of 12 gradients (up to 200 nM final concentration) at 50 μL/well, added to 96-well plates, and mixed well with huTL1A-his-Biotin protein and 293-huDR3 cells. Incubate at 4° C. for 30 min. Cells are washed twice in PBS to remove unbound antibodies to be tested. Add 100 μL/well of Streptavidin-PE secondary antibody and incubate at 4° C. for 30 min. Centrifuge at 300 g for 5 min and wash the cells twice in PBS to remove unbound secondary antibody. Finally, the cells were resuspended in 200 μl PBS, and the ability of the anti-TL1A antibody to block the binding of TL1A protein to 293-huDR3 cells was determined by a Beckman Coulter CytoFLEX flow cytometer. The resulting data were fitted and analyzed by GraphPad Prism software. The results are shown in FIG. 8 and Table 14, and the antibodies to TLA0015, TLA0118, TLA0140, TLA0148, TLA0162, TLA00165, TLA0167, and TLA0238 all have the ability to block the binding of TL1A-his protein to 293-DR3 cells.

TABLE 14
Anti-TL1A antibody blocking activity-IC50 values

| Abs | IC50 (nM) | Bottom (MFI) |
|---|---|---|
| RVT-3101 | 16.68 | 1668 |
| (Analogue) | | |
| MK-7240 | 85.05 | 10492 |
| (Analogue) | | |
| TLA0015 | 12.10 | 1326 |
| TLA0118 | 26.21 | 5403 |
| TLA0238 | 12.19 | 2855 |
| TLA0140 | 35.79 | 4416 |
| TLA0148 | 28.08 | 3886 |
| TLA0162 | 17.27 | 1802 |
| TLA0165 | 19.15 | 2826 |
| TLA0167 | 38.48 | 4581 |

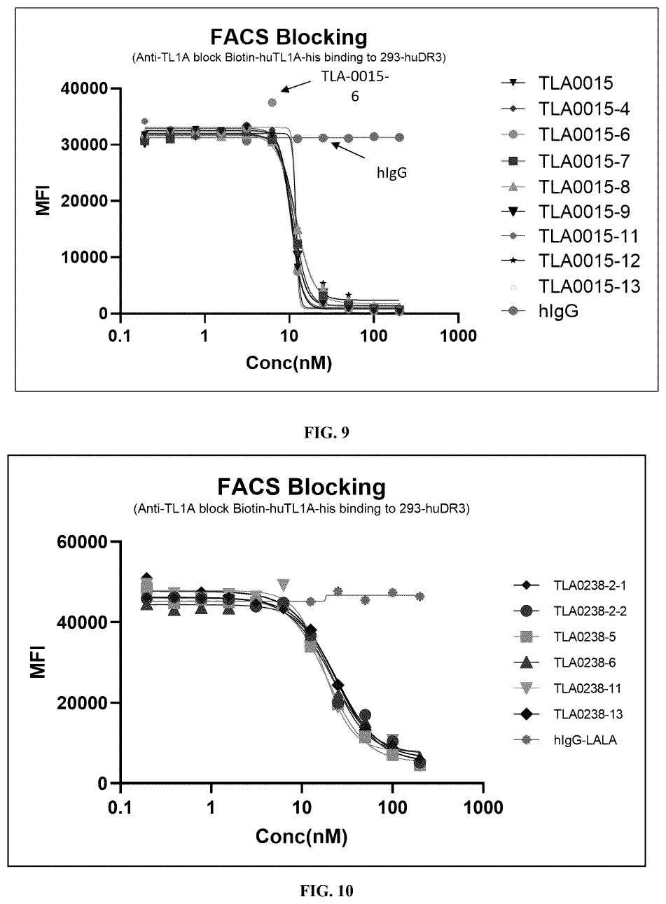

The blocking effect of TLA0015 humanized antibody on human TL1A-his trimeric protein was detected, and the specific results are shown in FIG. 9 and Table. 15

TABLE 15
TLA0015 humanized antibody blocking activity-IC50 values

| | | Bottom |
|---|---|---|
| Abs | IC50 (nM) | (MFI) |
| TLA0015 | 10.52 | 263 |
| TLA0015-4 | 12.03 | 327 |
| TLA0015-6 | 11.83 | 300 |
| TLA0015-7 | 11.29 | 605 |
| TLA0015-8 | 11.89 | 1059 |
| TLA0015-9 | 10.76 | 519 |
| TLA0015-11 | 10.33 | 363 |
| TLA0015-12 | 11.92 | 916 |
| TLA0015-13 | 11.01 | 642 |

The blocking effect of TLA0238 humanized antibody on human TL1A-his protein was detected, and the specific results are shown in FIG. 10 and Table 16.

TABLE 16
TLA0238 humanized antibody blocking activity-IC50

| Abs | IC50 (nM) | Bottom (MFI) |
|---|---|---|
| TLA0238-2-1 | 23.92 | 6053 |
| TLA0238-2-2 | 21.06 | 5117.9 |
| TLA0238-5 | 19.29 | 4534.9 |
| TLA0238-6 | 22.29 | 6333.3 |
| TLA0238-11 | 17.64 | 4485.9 |
| TLA0238-13 | 22.99 | 5570.1 |

### Example 11 Fluorescent Reporter Gene Method to Detect the Ability of TL1A Antibody to Inhibit NF-κB Signaling Pathway

A luciferase reporter gene-based method was used to test the ability of the plurality of antibodies of the present invention obtained by example 6 to inhibit the NF-κB signaling pathway in soluble or membrane-bound TL1A-activated TF-1 (endogenously expressing DR3) cells. The TF-1-NFκB-Luc cell line was purchased from Genomeditech Biotechnology Ltd (GM-C30289, H_TNFSF15 (TL1A) Reporter Cell Line) and screened using NF-κB-Luciferase lentiviral infection of TF-1 cells.

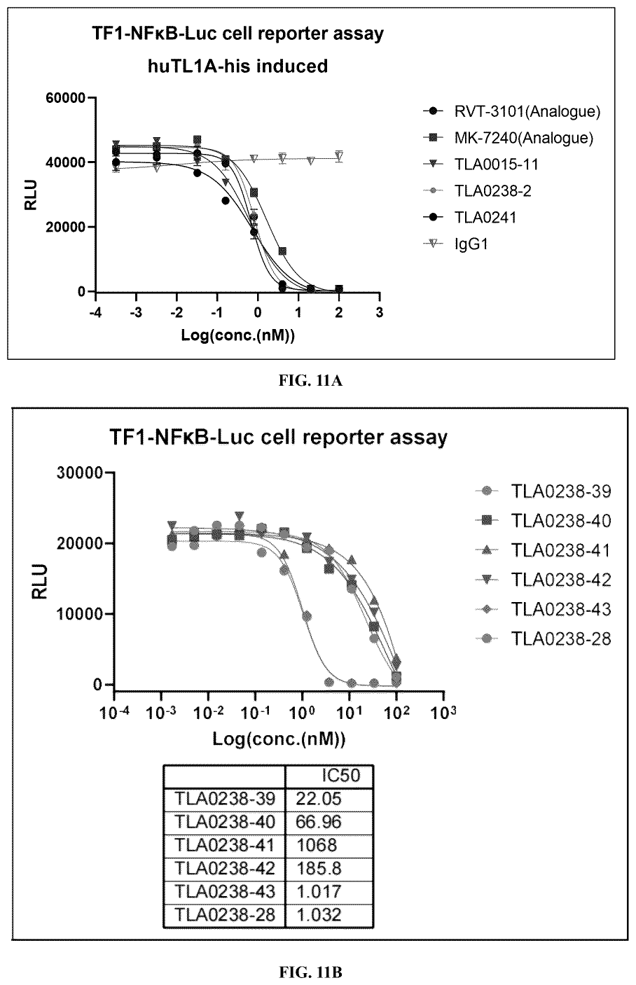

Fluorescent reporter gene method for soluble TL1A protein activation. In this study, TF1-NFκB-luc cells were used to detect the biological activity of anti-TL1A antibody to inhibit the DR3-NFκB signaling pathway activated by soluble TL1A. In this experiment, 3× of the analytical concentration of huTL1A-his antigen solution was prepared with analytical buffer (RPMI 1640 medium+1% FBS) at 0.9 μg/ml (final concentration of 0.3 μg/ml) and added to a white 96-well flat-bottom plate at 40 μl/well. Prepare 3× analytical concentrations of anti-TL1A antibody with a maximum concentration of 300 nM (final concentration of 100 nM), 5-fold serial dilution for a total of 8 concentration points. After the antibody is diluted, 40 μl/well is added to the above white 96-well flat-bottom plate, and then the white 96-well flat-bottom plate is placed in a carbon dioxide incubator to pre-incubate the TL1A antibody and antigen for 1 h. During the antibody and antigen pre-incubation, TF1-NFκB-luc cells were centrifuged (300 g, 5 min) and then resuspended with analysis buffer and counted, and the cell density was adjusted to 5E5 cells/ml. After the antigen and antibody incubation time is over, take out the white 96-well flat-bottom plate, inoculate TF1-NFκB-luc cells with adjusted density at 40 μl/well, and continue to incubate in a carbon dioxide incubator for 6 h. At the end of the incubation, remove the white 96-well flat-bottom plate from the incubator, add 60 μl of Bio-Lite Luciferase Assay reagent (Vazyme, DD1201-02) to each well, shake slightly for 5 minutes, and read the RLU value on a multimode microplate reader. Graphpad was used to plot the logarithm of the sample concentration with the measured signal values, and the results are shown in FIG. 11A and Table 17: TLA0015-11, TLA0238-2, and TLA0241 antibodies all have good activity to inhibit the NF-κB signaling pathway activated by soluble TL1A. In addition, the results of the inhibitory activity of other humanized antibodies of TLA0238 are shown in FIGS. 11B, 11C and 11D. As shown in the figures, the inhibitory activity of antibodies TLA0238-43, TLA0238-44, TLA0238-45, TLA0238-46, TLA0238-47, TLA0238-48, TLA0238-49 and TLA0238-50 are all very good.

TABLE 17
Anti-TL1A antibody inhibits the NF-κB signaling
pathway-IC50 values activated by soluble TL1A

| Abs | IC50 (nM) |
|---|---|
| RVT-3101 (Analogue) | 0.674 |
| MK-7240 (Analogue) | 1.622 |
| TLA0015-11 | 0.631 |
| TLA0238-2 | 0.829 |
| TLA0241 | 0.725 |

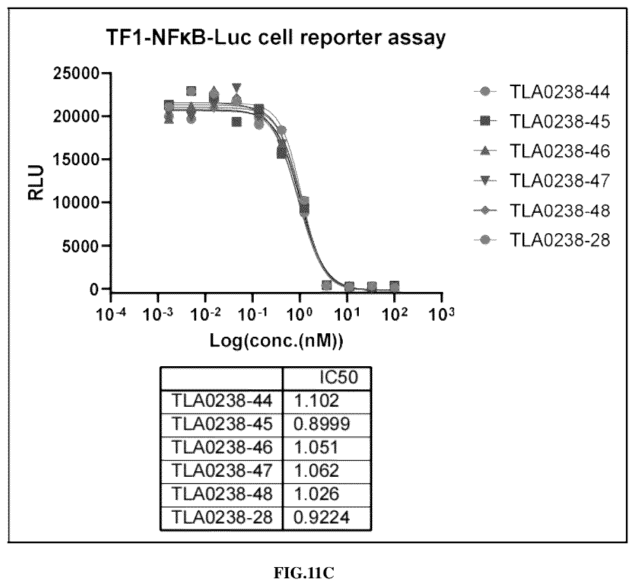

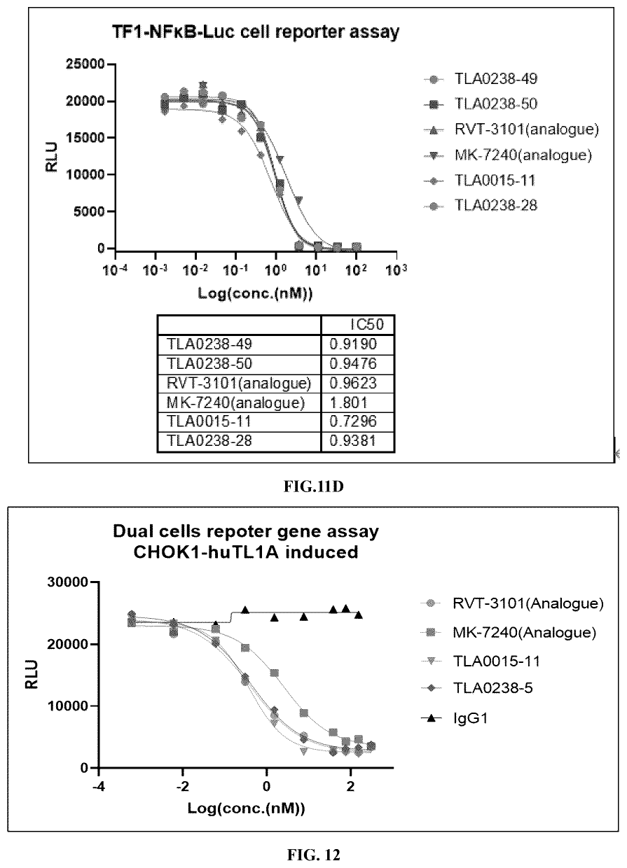

Fluorescent reporter method for membrane-bound TL1A activation. In this study, CHO-K1-TL1A cells (overexpressing human full-length TL1A were constructed on CHO-K1 cells) and TF1-NFκB-luc cells were used to evaluate the biological activity of anti-TL1A antibody against membrane-bound TL1A-DR3-NFκB signaling pathway. CHO-K1-TL1A cells were digested, centrifuged (300 g, 5 min), resuspended with F-12K complete medium and counted, the cell density was adjusted to 5E5 cells/ml, and 100 μl/well seeded in white 96-well flat-bottom plates were cultured overnight. The next day, prepare 2× analytical concentrations of anti-TL1A antibody with analytical buffer (RPMI 1640 medium+1% FBS) at a maximum concentration of 200 nM (final concentration of 100 nM).), 5-fold serial dilution for a total of 8 concentration points. After diluting the antibody, remove the white 96-well flat-bottom plate inoculated with CHO-K1-TL1A cells, aspirate the culture supernatant, and add 50 μl of the above diluted antibody to each well. Continue to pre-incubate the white 96-well flat-bottom plate in a CO2 incubator for 30 min. During the pre-incubation, TF1-NFκB-luc cells were centrifuged (300 g, 5 min) and then resuspended with analysis buffer and counted, and the cell density was adjusted to 1E6 cells/ml. After the pre-incubation time is over, remove the white 96-well flat-bottom plate and inoculate TF1-NFκB-luc cells with adjusted density at 50 μl/well. Place the white 96-well flat-bottom plate in a carbon dioxide incubator and continue to incubate for 6 h. At the end of the incubation, remove the white 96-well flat-bottom plate from the incubator, add 50 μl of Bio-Lite Luciferase Assay reagent to each well, and read the RLU value on a multimode microplate reader after 5 min of slight shaking. The results of the logarithm of the sample concentration and the measured signal value were shown in FIG. 12 and Table 18, and both TLA0015-11 and TLA0238-5 antibodies had good activity in inhibiting the NF-κB signaling pathway activated by membrane-bound TL1A.

TABLE 18
Anti-TL1A antibody inhibits membrane-
bound TL1A-activated NF-κB signaling

| Abs | EC50 (nM) |
|---|---|
| RVT-3101 | 0.393 |
| (Analogue) | |
| MK-7240 (Analogue) | 2.450 |
| TLA0015-11 | 0.363 |
| TLA0238-5 | 0.401 |

### Example 12 Detection of the Ability of Anti-TL1A Antibody to Inhibit TL1A-DR3-Activated Caspase3/7

A method based on the Caspase-Glo® 3/7 Assay System Activity Detection Kit (Promega, Cat #G8092) was used to assay the ability of the plurality of antibodies of the present invention, prepared by example 6, to inhibit the TL1A-induced apoptosis signaling pathway in TF-1 (endogenously expressing DR3) cells. The inhibitory capacity was determined by comparing the curves of caspase3/7 activity of TF-1 cells inhibited by different antibody concentrations.

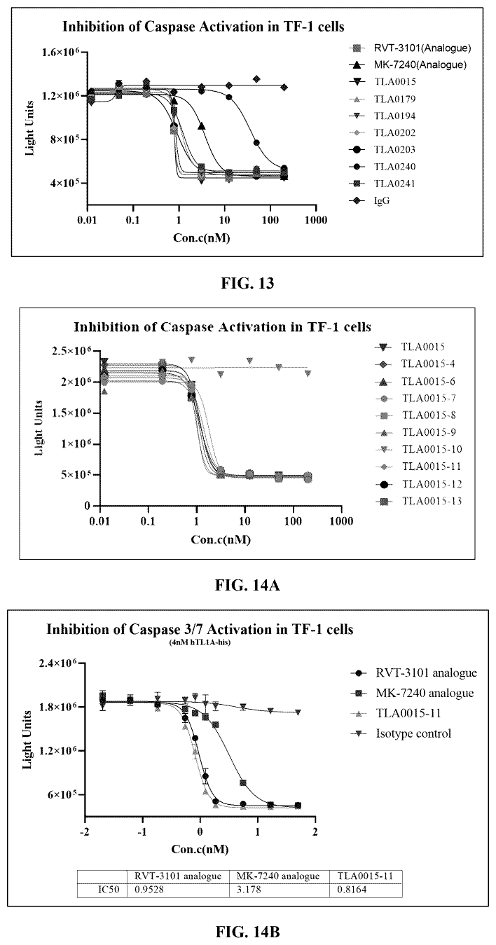

The huTL1A-his protein was configured to a 4× working concentration of 16 nM: the Cycloheximide small molecule drug was configured to a 4× working concentration of 10 μg/ml. 25 μl of the mixture was added to each well in a white-bottomed 96-well plate. Antibody starting concentration was configured as 10 nM, 2-fold dilution, 8 concentrations, 25 μl of each in a white-bottomed 96-well plate. Pre-incubate the above mixture at 37° C. for 1h. At the same time, centrifuge the TF-1 cells (300 g for 5 min) and resuspend them with buffer (1640 medium+10% FBS) and count them, and adjust the cell density to 1E6 cells/ml. When the incubation time of the antigen-antibody is over, take out the white 96-well plate, inoculate the TF-1 cells according to the adjusted density of 50 μl/well, and add 25 μl to each well in the white 96-well plate. After incubation, take out the white 96-well flat-bottom plate, inoculate with the adjusted density of TF-1 cells at 50 μl/well, and continue to incubate for 6 h in a carbon dioxide incubator. After incubation, take out the plate from the incubator and equilibrate the plate at room temperature for about 30 min, and take out the Caspase-Glo® 3/7 Assay System reagent beforehand and equilibrate the plate at room temperature. Add Caspase-Glo® 3/7 Assay System reagent, 50 μl per well, shake the shaker at 300 rpm for 30 s, and then let it stand for 15-30 min to make the cells fully lysed, and then read the RLU value on the multifunctional enzyme labeling instrument, and then make a four-parameter fit plot with the measured RLU value after taking the logarithm of the sample concentration using the Graphpad, the results are shown in FIG. 13 and Table 19. The results are shown in FIG. 13 and Table 19. TLA0015, TLA0179, TLA0194, TLA0202, TLA0203 and TLA0241 antibodies all functioned well to inhibit Caspase3/7 activity.

TABLE 19
Inhibition of TL1A-activated caspase3/7
activity by anti-TL1A antibody-IC50

| | IC50 |
|---|---|
| Abs | (nM) |
| RVT-3101 | 0.79 |
| (Analogue) | |
| MK-7240 | 3.70 |
| (Analogue) | |
| TLA0015 | 0.80 |
| TLA0179 | 1.10 |
| TLA0194 | 0.84 |
| TLA0202 | 0.79 |
| TLA0203 | 0.91 |
| TLA0240 | 36.20 |
| TLA0241 | 1.22 |

To test the ability of TLA0015 humanized antibodies to inhibit the TL1A-induced apoptosis signaling pathway in TF-1 cells. TLA0015 humanized antibodies (FIG. 14 and Table 20) have a good function of inhibiting caspase 3/7 activity. In addition, the activity difference between the humanized antibody TLA0015-11 and the control antibody was compared. As shown in FIG. 14C, the inhibitory activity of TLA0015-11 was superior to that of the control antibodies RVT-3101 and MK-7240.

TABLE 20
Inhibition of TL1A-activated caspase3/7
activity by TLA0015 humanized antibody

| Abs | IC50 (nM) |
|---|---|
| TLA0015 | 1.16 |
| TLA0015-4 | 1.15 |
| TLA0015-6 | 1.10 |
| TLA0015-7 | 1.80 |
| TLA0015-8 | 1.37 |
| TLA0015-9 | 1.25 |
| TLA0015-10 | 2.40 |
| TLA0015-11 | 1.03 |
| TLA0015-12 | 1.16 |
| TLA0015-13 | 0.98 |

### Example 13 Detection of the Inhibitory Effect of Anti-TL1A Antibody on T Cells

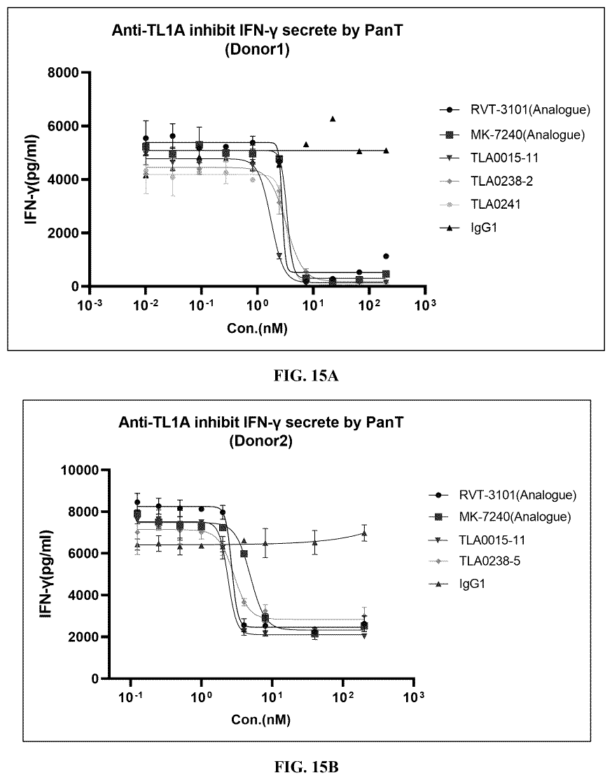

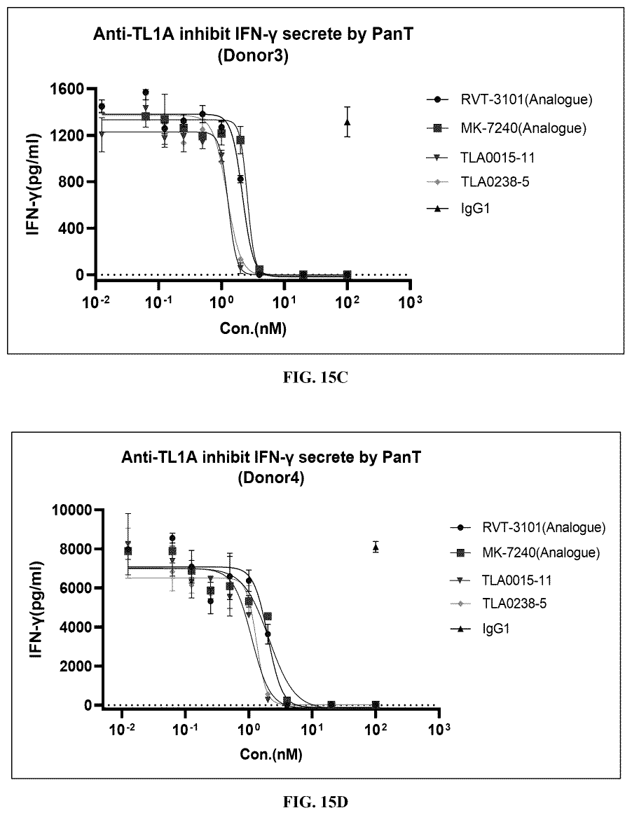

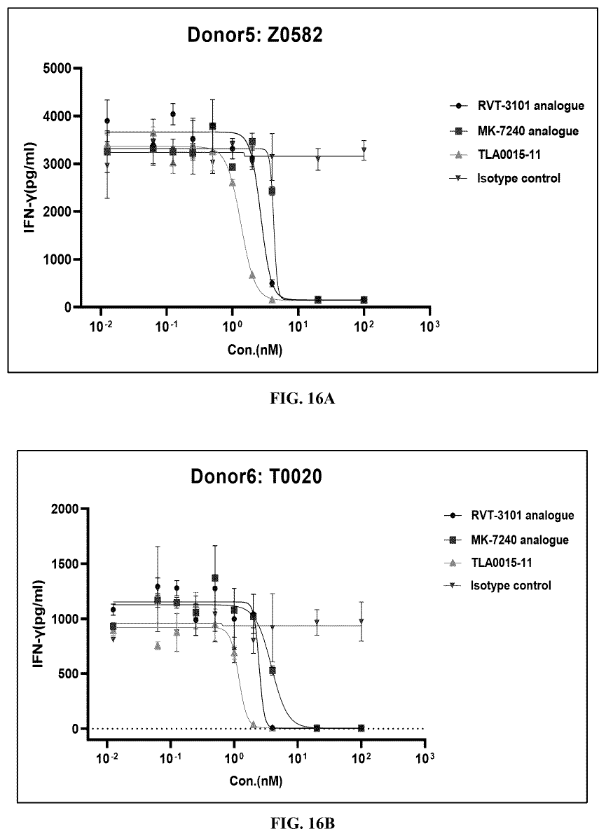

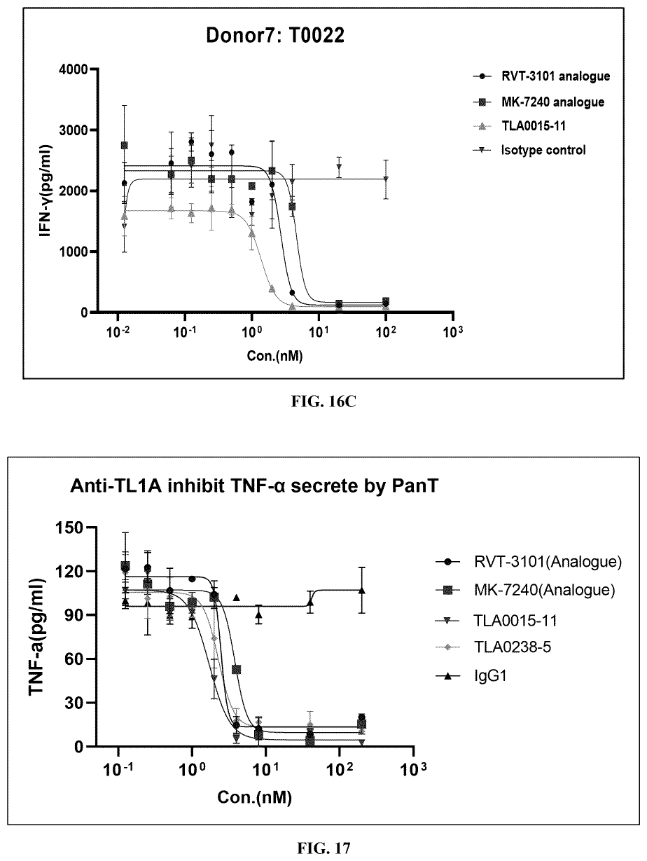

In this study, the inhibitory ability of anti-huTL1A antibody to immune cell activation was evaluated by measuring the amount of cytokines in the culture supernatant of CD3+ T cells isolated from fresh healthy human peripheral blood. Fresh healthy human PBMCs (purchased from Saili Bio) were centrifuged (500 g for 5 min) and the supernatant was discarded, and the cells were resuspended with PBS (containing 2% FBS+5 mM EDTA), counted, and the cell density was adjusted to 5E7 cells/mL. Use the CD3 negative selection kit (Biolegend, Cat #480131) to sort T cells according to the kit instructions. T cells were washed twice with PBS, centrifuged at 500 g each for 5 min, and the supernatant was discarded. Resuspend T cells with RPMI 1640 complete medium, count, adjust the cell density to 1.5E6 cells/mL, 100 μl/well, and add to a 96-well plate. Complete culture with RPMI 1640 was prepared with TL1A (final concentration 10 nM), IL-12 (final concentration 0.5 ng/ml), IL-18 (final concentration 5 ng/mL) mixed solution, 50 μl/well, and added to a 96-well plate. Dilute the antibody to be tested to 800 nM, followed by a 3-fold stepwise dilution of 10 gradients (final concentration up to 200 nM), 50 μl/well add to a 96-well plate. The 96-well plate was placed in a carbon dioxide incubator, incubated for 24 hours, removed, centrifuged at 500 g for 5 minutes, and the supernatant was aspirated and placed on ice for later use. The amount of cytokine IFN-γ in the supernatant was detected using the BD™ Cytometric Bead Array (human IFN-γ) kit. Prepare the standard according to the instructions, dilute the sample, 50 μl/well per well, and add to the 96-well plate. Prepare Capture beads solution at 50 μl/well per well, add to a 96-well plate, and incubate the standard/sample with the Capture beads solution for 1 h. Dilute the detection antibody solution according to the instructions, 50 μl/well per well, add to a 96-well plate, and incubate for a total of 2 h. Centrifuge at 300 g for 5 min, discard the supernatant, and wash with wash buffer. Centrifuge again at 300 g for 5 min, discard the supernatant, resuspend with wash buffer, 70 μl/well, and determine the amount of cytokine IFN-γ by Beckman CytoFLEX flow cytometer. The obtained data were fitted and analyzed by GraphPad Prism software, and the results are shown in FIG. 15, and both TLA0015-11 and TLA0238-5 have good inhibition activity of IFN-γ in PBMCs in different healthy people. The inhibitory activity of TLA0015-11 on IFN-γ secretion by T cells was tested in several different PBMCs at the same time, and the results are shown in FIGS. 16A-16C. TLA0015-11 has a very good inhibitory activity against IFN-γ in three different PBMCs, and the inhibitory activity is better than that of the control antibodies RVT-3101 (analog) and MK-7240 (analog).

The amount of cytokine TNF-α in the supernatant was also detected using the BD™ Cytometric Bead Array Human TNF Flex Set. Prepare the standard according to the instructions, dilute the sample, 50 μl/well per well, and add to a 96-well plate. Prepare Capture beads solution at 50 μl/well per well, add to a 96-well plate, and incubate the standard/sample with Capture beads solution for 1 h. Dilute the detection antibody solution according to the instructions, 50 μl/well per well, add to a 96-well plate, and incubate for a total of 2 h. Centrifuge at 300 g for 5 min, discard the supernatant, and wash with wash buffer. Centrifuge again at 300 g for 5 min, discard the supernatant, resuspend with 70 μl wash buffer, and determine the cytokine content by Beckman CytoFLEX flow cytometer. The resulting data were fitted and analyzed by GraphPad Prism software. As shown in FIG. 17, both TLA0015-11 and TLA0238-5 have good activity against TNF-α.

### Example 14 Detection of Cross-Reactivity of Anti-TL1A Antibody with Proteins of the Same Family

The experiment is based on an ELISA assay to detect the binding ability of a plurality of antibodies prepared by example 6 of the present invention to other proteins of the same family as TL1A. The specificity of the TL1A antibody is determined by detecting whether the antibody binds to the TL1A family of proteins. TL1A homo proteins include: FasL (36% homology to TL1A sequence), TRAIL (35% homology to TL1A sequence), LIGHT (31% homology to TL1A sequence), TNF-β (30% homology to TL1A sequence), and TNF-α (29% homology to TL1A sequence).

TL1A homologous family proteins: FasL (Acrobiosystems, Cat #FAL-H5241), TRAIL (Acrobiosystems, Cat #TRL-H5259), LIGHT (Acrobiosystems, Cat #LIT-H5242), TNF-β (Acrobiosystems, Cat #TNB-H52H3) and TNF-α (Acrobiosystems, Cat #TNA-H5228) were purchased for the preparation of positive control antibodies to the same family of proteins. FasL-his, TRAIL-his, LIGHT-his, TNF-β-his and TNF-α-his proteins were diluted to 2 μg/ml in PBS at 100 μl/well, respectively, added to the enzyme labeled plates (coated with an equal amount of BSA as a control), and left at 4° C. overnight. The coating solution was removed, 5% BSA blocking solution was added at 200 μl/well and was left at room temperature for 2 hours. Remove the blocking solution, wash three times with 250 μl/well of 0.5%. PBST, then dilute anti-TL1A antibody and positive antibodies of each protein of the same family to 100 nM with the blocking solution, and add them sequentially to the blocked plates with 100 μl/well, and leave at room temperature for 1 hour. The plate was washed 3 times with PBST (residual droplets were removed with absorbent paper), and HRP-labeled goat anti-human IgG antibody was added at 100 μl/well and left at room temperature for 45 min. Wash the plate with 0.5%. PBST for 5 times, in 100 μl/well, add TMB solution, and leave it at room temperature and avoid light for 5 minutes, add termination solution in 100 μl/well to terminate the color development reaction of the substrate, and read the OD value at 450 nm with enzyme marker. The results are shown in Table 21: Anti-TL1A specific monoclonal antibodies TLA0015, TLA0238 and TLA0241 all did not bind to other proteins of the same family.

TABLE 21
Anti-TL1A antibody binding detection to the same family protein

| Abs | FasL | TRAIL | LIGHT | TNF-β | TNF α |
|---|---|---|---|---|---|
| TLA0015 | 0.05 | 0.05 | 0.05 | 0.05 | 0.05 |
| TLA0238 | 0.05 | 0.05 | 0.05 | 0.05 | 0.05 |
| TLA0241 | 0.05 | 0.05 | 0.05 | 0.05 | 0.05 |
| Cytokine Abs | 3.30 | 2.64 | 3.29 | 1.81 | 3.65 |

### Example 15 Detection of Anti-TL1A Antibody Tm Value Detection

The experiment is based on a Panta instrument to detect the Tm values of a plurality of antibodies prepared by example 6 of the present invention. The Panta instrument includes micro-differential scanning fluorescence (nanoDSF) technology, which tracks the folding state of a protein by detecting its endogenous fluorescence. The ratio of the fluorescence signal changes with increasing temperature or chemical denaturant concentration, thus determining the protein stability parameter Tm value. The specific Tm values of each antibody are shown in Table 22.

TABLE 22
The body Tm value of the anti-TL1A antibody

Tm(° C.)

| Abs | Tm1(° C.) | Tm2(° C.) |
|---|---|---|
| RVT-3101 (Analogue) | 71.65 | 79.21 |
| MK-7240 (Analogue) | 65.05 | 81.44 |
| TLA0015-11 | 75.62 | NA |
| TLA0238-2-1 | 69.92 | NA |
| TLA0238-5 | 69.60 | NA |

### Example 16 Detection of ADCC Activity of Anti-TL1A Antibody TLA0015-11 by Fluorescent Reporter Gene Method

In this experiment Jurkat-ADCC fluorescent reporter gene cells were used to detect the ADCC activity of anti-TL1A antibody TLA0015-11. The effector cells used in this study were Jurkat-ADCC cells overexpressing FcγRIIIa and downstream fluorescent reporter genes, and the ADCC activity of the TLA0015-11 antibody was reflected by determining the expression of fluorescent reporter genes in the effector cells. The Jurkat-ADCC cell line was purchased from Triple Excellence Bio (XHB604, Human FcgR3a/CD16a NF-AT-Jurkat Cell Line).

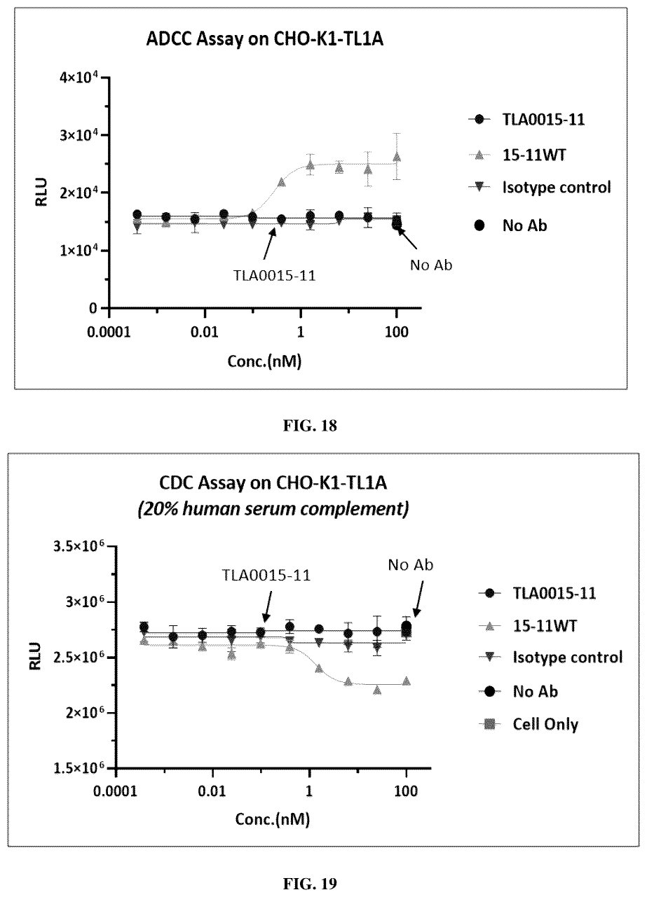

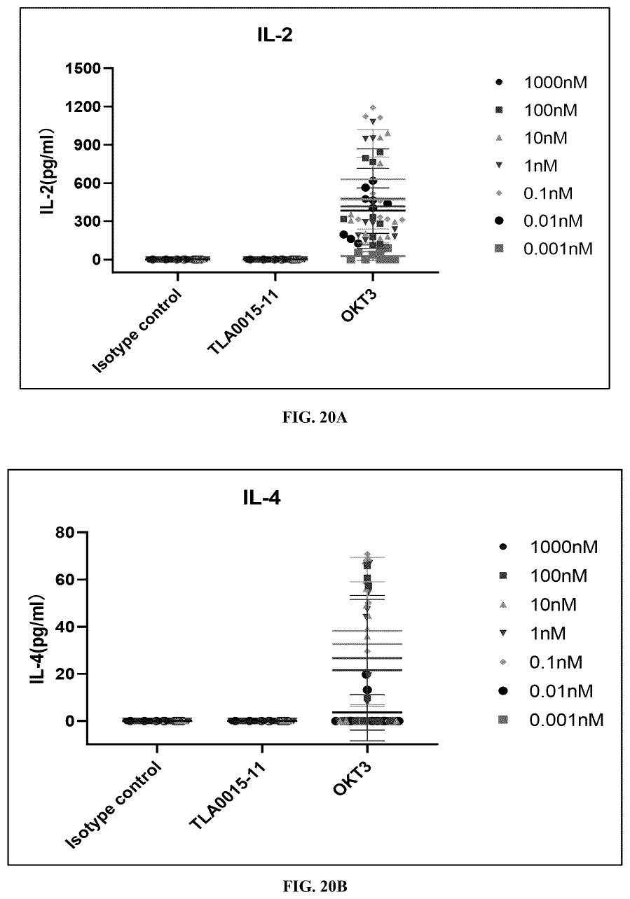

CHO-K1-TL1A cells were digested and centrifuged, and the cell density was adjusted to 2E5 cells/mL using analytical buffer (phenol red-free RPMI1640 medium+4% FBS+1% Penicillin Streptomycin), and the target cells were inoculated into white 96-well flat-bottomed plates with 50 μl per well. 3× analytical buffer was used to prepare 3× analytical concentration of TLA0015-11 monoclonal antibody, positive control antibody 15-11WT (Fc is human IgG1 wild type Fc) and negative control antibody Isotype control, the highest concentration point is 300 nM (final concentration is 100 nM), 4-fold gradient dilution, a total of 10 concentration points. After the antibody was diluted, it was added to the white 96-well flat-bottomed plate as described above at 50 μl/well. The density of Jurkat-ADCC effector cells was then adjusted to 1E6 cells/mL with analytical buffer, and 50 μl/well was added to the white 96-well flat-bottomed plate and incubated in a carbon dioxide incubator for 6 h. After incubation, the white 96-well flat-bottomed plate was taken out of the incubator, and 40 μl of Bio-Lite Luciferase Assay Reagent was added to each well. (Vazyme, DD1201-02) per well, and read the RLU value on a multifunctional enzyme marker after 5 minutes of gentle shaking. A four-parameter fit plot of the sample concentration logarithmically versus the measured signal value was performed using Graphpad, and the results are shown in FIG. 18: anti-TL1A antibody TLA0015-11 has no ADCC activity.

### Example 17 CDC Activity of Anti-TL1A Antibody TLA0015-11 Detected by Human Complement Serum

In this experiment, human complement serum was used to detect whether anti-TL1A antibody TLA0015-11 has CDC activity at in vitro level.

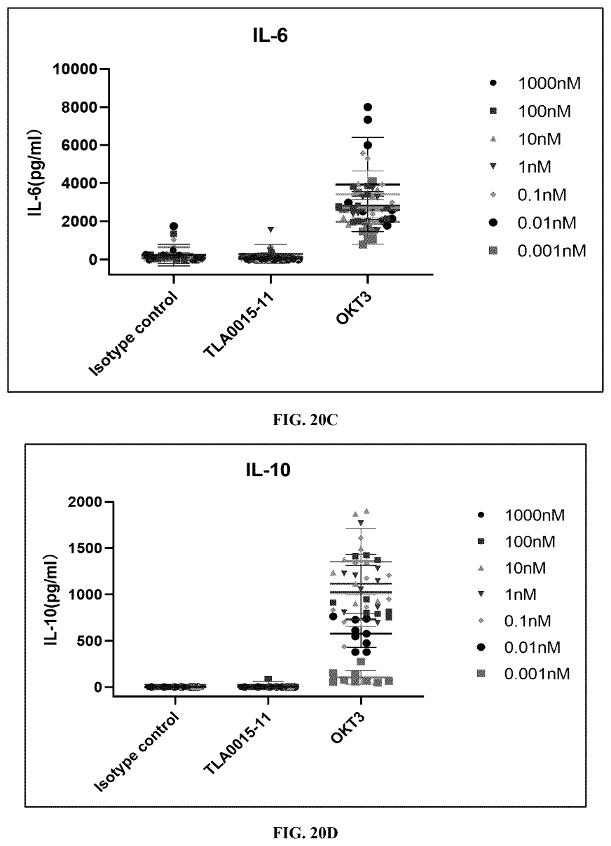

CHO-K1-TL1A cells were digested and centrifuged, and the cell density was adjusted to 6.7E5 cells/mL using analytical buffer (phenol red-free RPMI1640 medium+4% FBS+1% Penicillin Streptomycin), and target cells were inoculated into white 96-well flat-bottomed plates with 30 μl per well. 2× of analytical buffer was used to prepare the analyzed concentration of anti-TL1A antibody TLA0015-11, positive control antibody 15-11WT and negative control antibody Isotype control, the highest concentration point is 200 nM (final concentration is 100 nM), 4-fold gradient dilution, a total of 10 concentration points. After the antibody was diluted, it was added to the above white 96-well flat-bottom plate at 50 μl/well. At the same time, Normal Human Serum Complement (QuidelOrtho, A113) was added to the white 96-well plate at 20 μl/well, and the white 96-well plate was incubated in a carbon dioxide incubator for 4 h. At the end of the incubation, the white 96-well plate was removed from the incubator, and 50 μl of the Normal Human Serum Complement (QuidelOrtho, A113) was added to each well. Cell Counting-Lite 2.0 Luminescent Cell Viability Assay Reagent (Vazyme, DD1101-02) was added to each well, and the RLU values were read on a multifunctional enzyme marker after 5 minutes of gentle shaking. A four-parameter fit plot of the sample concentration logarithmically plotted against the measured signal value using Graphpad was performed, and the results are shown in FIG. 19: monoclonal antibody TLA0015-11 has no CDC activity.

### Example 18 Detection of Cytokine Release Activity Mediated by Anti-TL1A Antibody TLA0015-11

This experiment was performed to evaluate whether TLA0015-11 monoclonal antibody can mediate cytokine release syndrome using human peripheral blood single nucleated cells in vitro. The TLA0015-11 antibody and negative control antibody IgG1-LALA were diluted with 1640 complete medium to prepare 2× analyzed concentrations of the antibody with a maximum concentration of 2 μM (final concentration of 1 μM), and the positive control antibody was OKT3 with a maximum concentration of 200 nM (final concentration of 100 nM). All antibodies were diluted according to a 10-fold gradient with a total of 6 concentration points, and 3 replicate wells were made for each concentration point. The diluted antibodies were added to 96-well round bottom plates at 50 μl per well. PBMC from three different donors were resuscitated, quickly thawed and transferred to centrifuge tubes with 9 ml 1640 complete medium beforehand, the supernatant was discarded after centrifugation (400 g, 5 min), fresh 1640 complete medium was added to resuspend and counted, and the cell density was adjusted to be 6E6 cells/ml, and the cell density was adjusted to be 6E6 cells/ml. Add 50 μl per well into the above 96-well round bottom plate (3E5 PBMC per well), and incubate in a CO2 incubator for 24 h. After incubation, remove the 96-well round bottom plate, centrifuge at 400 g for 5 min, and pipette 50 μl of culture supernatant from each well into a new 96-well V-bottom plate, then incubate in accordance with the BD™ Cytometric Bead Array (CBA) Human Th1/Th1 Array. (CBA) Human Th1/Th2 Cytokine Cytometric Bead Array (CBA) Kit II (BD, Cat #551809) instructions, and then processed to detect the content of multiple cytokines (IL-2, IL-4, IL-6, IL-10, TNF-α, IFN-γ) in cell supernatants. The samples were collected on a flow cytometer (Agilent, NovoCyte Penteon), analyzed by FCAP Array v3 software, and finally plotted against the antibody concentration versus the concentration of each cytokine by Graphpad, and the results are shown in FIGS. 20A-20F. The effect of TLA0015-11 monoclonal antibody on IL-2, IL-4, IL-6, IL-10, the TNF-α, and IFN-γ cytokines were not stimulated.

### Example 19 Biacore Detection of the Affinity of Anti-TL1A Antibodies to Different Fc Receptors and Complement Proteins

The affinity of the TLA0015-11 antibody to different Fc receptors and complement C1q protein was tested using the Biacore biomolecular interaction instrument.

In the detection experiment, the CM5 chip captured anti-his antibodies and then captured different Fc receptor proteins, including Human FcγRI (ACRO, FCA-H52H1) protein, Human FcγRIIa (H131) (ACRO, CD1-H5223) protein, Human FcγRIIa (R131) (ACRO, CDA-H5221) protein, Human FcγRIIb (ACRO, CDB-H5228) protein, Human FeyRIIIa (F158) (ACRO, CDA-H5220) protein, Human FcγRIIIa (V158) (ACRO, CD8-H52H4) protein, Human FcγRIIIb (NA1) (ACRO, CDB-H5227) protein, Human FcγRIIIb (NA2) (ACRO, CDB-H5222) protein, complement C1q (Complement Technology, A099) protein, and human FcRn (ACRO, FCM-H5286) protein. Two batches of TLA0015-11 antibody (batch 2784S241008Y and batch 0000165335) were used as analytes to measure binding activity to each protein. 15-11 WT antibody without Fc mutations (i.e., the Fc of TLA0015-11 in Example 6 was replaced with an Fc sequence without mutation) was also used as a control.

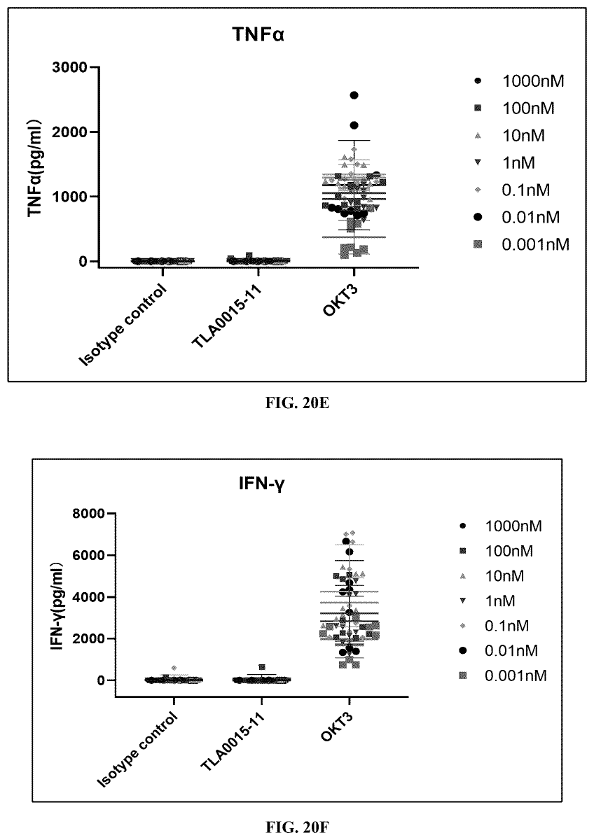

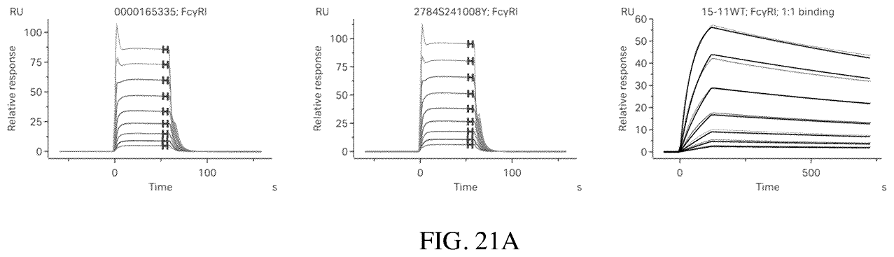

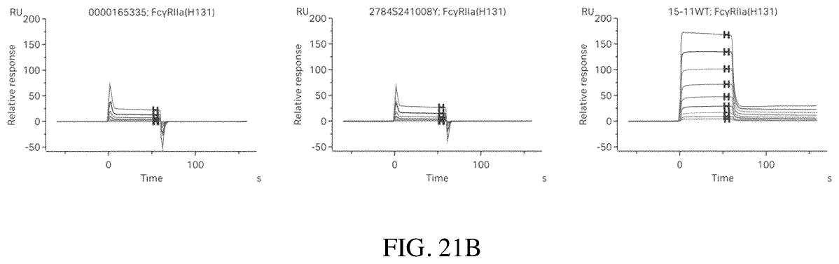

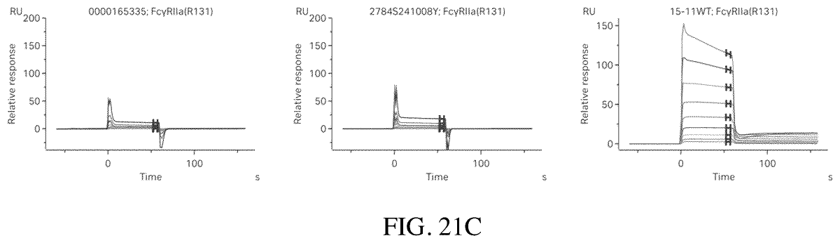

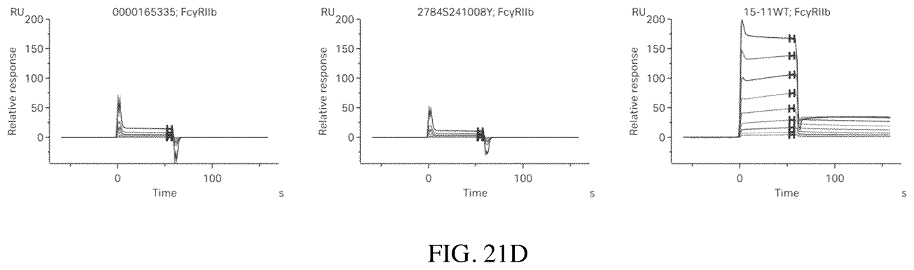

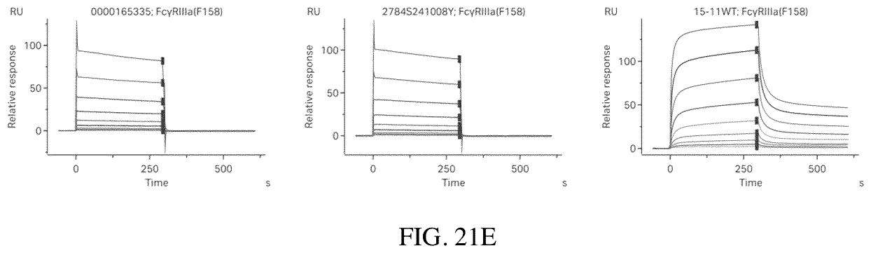

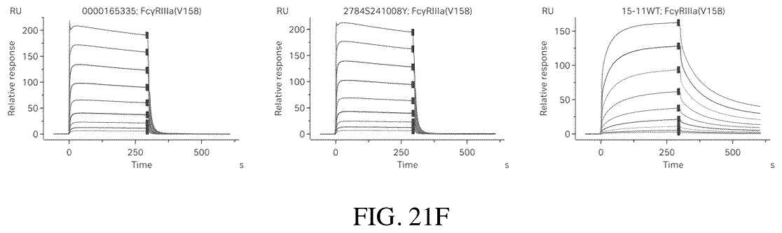

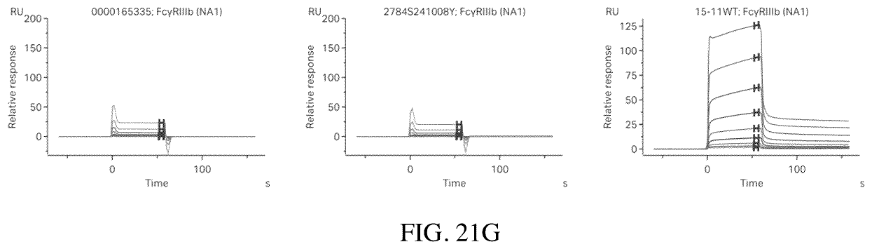

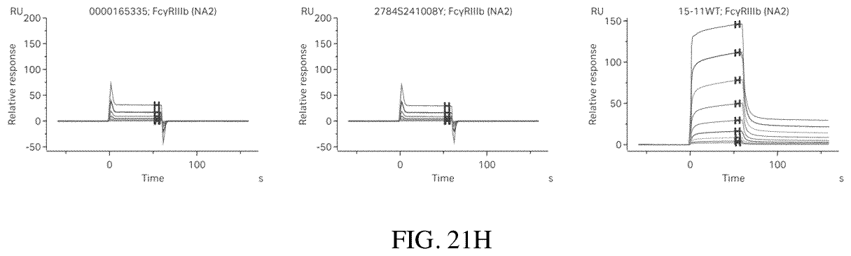

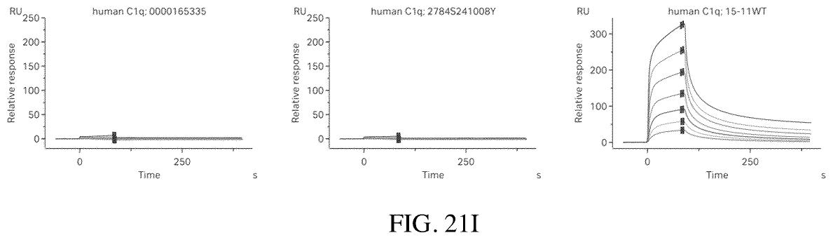

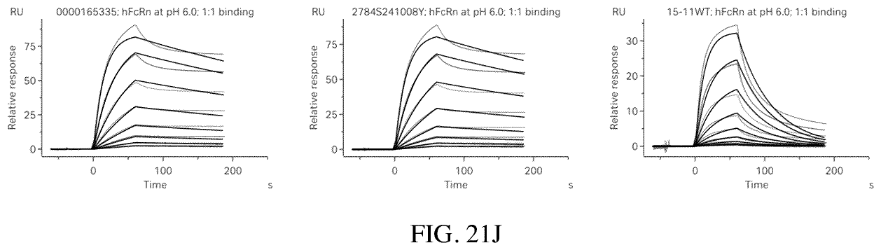

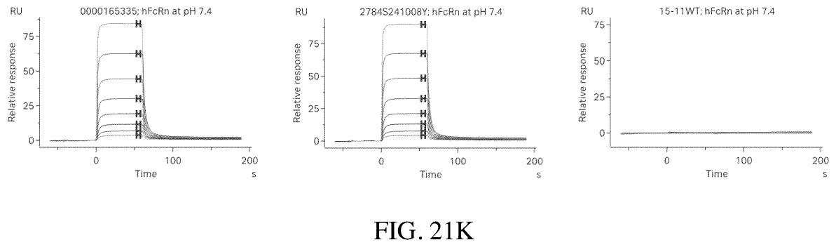

Binding and dissociation curves for different batches of TLA0015-11 antibody and the 15-11 WT antibody without Fc mutations to FeyRI, FcγRIIa (H131), FcγRIIa (R131), FcγRIIb, FcγRIIIa (F158), FcγRIIIa (V158), FcγRIIIb (NA1), FcγRIIIb (NA2), C1q. FcRn (pH 6.0), and FcRn (pH 7.4) are shown in FIGS. 21A-21K, and specific affinity values are shown in Table 23. The TLA0015-11 antibody has reduced affinity for various Fc receptors and complement proteins due to the introduction of the L234A/L235A (LALA) mutations in its Fc region. Consistent with expectations, TLA0015-11 antibody from different batches showed very weak or even no affinity for various Fcγ receptors and complement C1q proteins. In contrast, the 15-11 WT antibody, which did not undergo Fc mutations, showed moderate affinity for various Fc receptors and complement proteins, all stronger than that of the HX15001 antibody. The TLA0015-11 antibody, which incorporates the M252Y, S254T, and T256E (YTE) mutations in its Fc region, has increased its affinity for FcRn at low pH. Consistent with expectations, the TLA0015-11 antibody from different batches showed strong affinity for FcRn at pH 6.0, surpassing that of the 15-11 WT antibody.

TABLE 23
Affinity results of HX15001 antibody to
Fc receptors and complement C1q protein

| Protein | antibody | kₐ (1/Ms) | k_{d} (1/s) | K_{D} (M) |
|---|---|---|---|---|
| Human FcγRI | 0000165335 | NA | | 5.79E−06 |
| | 2784S241008Y | NA | | 6.04E−06 |
| | 15-11WT | 5.44E+05 | 4.69E−04 | 8.63E−10 |

| | | | |
|---|---|---|---|
| Human FcγRIIa | 0000165335 | NA | Weak or |
| (H131) | | | no binding |
| | 2784S241008Y | NA | Weak or |
| | | | no binding |
| | 15-11WT | NA | 2.61E−06 |
| Human FcγRIIa | 0000165335 | NA | Weak or |
| (R131) | | | no binding |
| | 2784S241008Y | NA | Weak or |
| | | | no binding |
| | 15-11WT | NA | 4.57E−06 |
| Human FcγRIIb | 0000165335 | NA | Weak or |
| | | | no binding |
| | 2784S241008Y | NA | Weak or |
| | | | no binding |
| | 15-11WT | NA | 9.10E−06 |
| Human FcγRIIIa | 0000165335 | NA | 3.41E−05 |
| (F158) | 2784S241008Y | NA | 3.62E−05 |
| | 15-11WT | NA | 8.38E−07 |
| Human FcγRIIIa | 0000165335 | NA | 8.11E−06 |
| (V158) | 2784S241008Y | NA | 7.55E−06 |
| | 15-11WT | NA | 2.00E−07 |
| Human FcγRIIIb | 0000165335 | NA | Weak or |
| (NA1) | | | no binding |
| | 2784S241008Y | NA | Weak or |
| | | | no binding |
| | 15-11WT | NA | 5.38E−06 |
| Human FcγRIIIb | 0000165335 | NA | Weak or |
| (NA2) | | | no binding |
| | 2784S241008Y | NA | Weak or |
| | | | no binding |
| | 15-11WT | NA | 4.10E−06 |
| Human C1q | 0000165335 | NA | Weak or |
| | | | no binding |
| | 2784S241008Y | NA | Weak or |
| | | | no binding |
| | 15-11WT | NA | 3.82E−08 |
| Human FcRn at | 0000165335 | NA | 1.19E−09 |
| pH 6.0 | 2784S241008Y | NA | 1.25E−09 |
| | 15-11WT | NA | 1.36E−08 |
| Human FcRn at | 0000165335 | NA | 1.75E−06 |
| pH 7.4 | 2784S241008Y | NA | 1.66E−06 |
| | 15-11WT | NA | Weak or |

no binding

### Example 20 Pharmacokinetics of Anti-TL1A Antibodies in hFcRn Transgenic Mice

In this experiment, B-hFcRn transgenic mice were used to study the pharmacokinetics of anti-TL1A antibodies. FcRn is the receptor for antibody IgG, and its binding is pH-dependent. In a weakly acidic environment, the IgG-Fc segment exhibits a high affinity for FcRn, while in a neutral or alkaline environment, the IgG-Fc segment exhibits a low affinity for FcRn. When IgG is non-specific pinocytosis by cells, it enters the endosome in an acidic environment, and shows a high affinity binding to FcRn on the endosomal membrane, and then is transported to the cell membrane by the circulating endosome, at this time, under alkaline conditions, the affinity between the IgG-Fc segment and FcRn is very low, and IgG is released from the FcRn into the blood circulation, thereby increasing the half-life of IgG. When M252Y, S254T and T256E mutations are introduced into IgG-Fc, the binding ability of Fc to FcRn receptors can be enhanced, and the half-life of IgG can be better extended. B-hFcRn transgenic mice (Catalog: 110001) were purchased from Biocytogen, and the mouse FcRn gene was knocked out on the C57BL/6 strain mice, and the human FcRn gene was knocked in, and the transgenic mouse only expressed the human FcRn receptor protein, but not the mouse FcRn receptor, so it can be used for metabolic kinetic studies of antibody drugs.

Female B-hFcRn transgenic mice with SPF grade (Catalog: 110001) were purchased from Biocytogen (Beijing) Pharmaceutical Technology Co., Ltd., and after the animals were quarantined, 10 mice were randomly assigned to each antibody, divided into 2 groups, 5 mice in each group, and blood was collected alternately. Each mouse is injected with 1 mg/kg of anti-TL1A antibody through the tail vein, followed by alternating blood sampling at time points in Table 23. Blood was collected by mandibular vein by collecting 20 μl/time point/piece, leaving it at room temperature for 1 h, and centrifugation (8000 rpm, centrifugation for 5 min) at room temperature. Recombinant human TL1A-his protein was diluted to 1 μg/ml with PBS at 100 μl/well, added to the plate (coated with an equal amount of BSA as a control), and incubated overnight at 4° C. Remove the coating solution, add blocking solution at 200 μl/well, and incubate for 2 h at room temperature. Remove the blocking solution and wash three times at 250 μl/well with 0.5%. PBST and pat dry for later use. Dilute the serum sample to the appropriate ratio using the blocking solution and add it to the blocked plate. The standard curves were used using RVT-3101, MK-7240, and TLA0015-11 starting at 100 nM with 2-fold dilutions for a total of 20 concentration gradients (maximum concentration of 100 nM), sequentially added to the blocked plate at 100 μl/well, and incubated for 1 h at room temperature. Wash the plate 3 times with 0.5%. PBST at 100 μl/well, add HRP-labeled goat anti-human IgG antibody, and incubate for 45 min at room temperature. Wash the plate 5 times with 0.5%. PBST, add TMB at 100 μl/well, incubate at room temperature in the dark for 5 min, at 100 μl/well, add stop solution, stop the substrate chromogenic reaction, read the OD value at 450 nm with a microplate reader, analyze the data with a GraphPad, plot and calculate the half-life (T1/2).

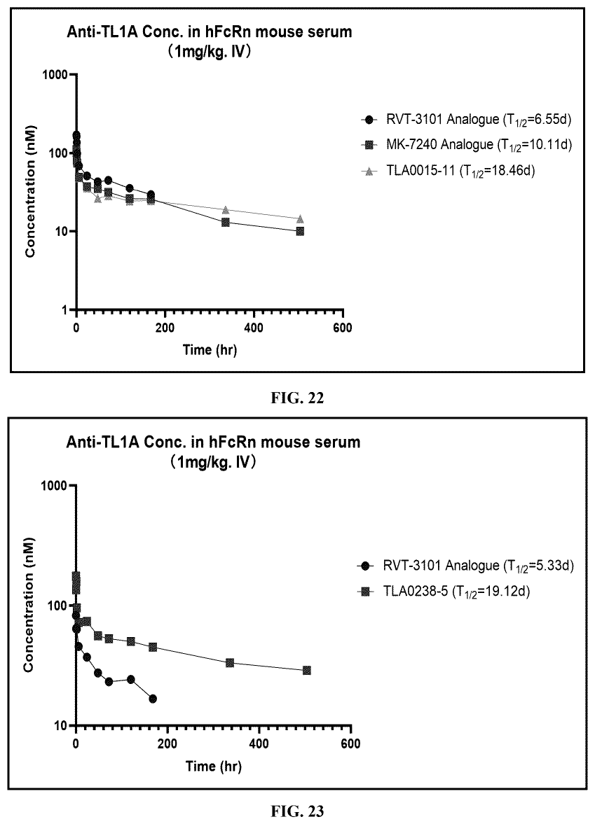

The results of the same batch of RVT-3101, MK-7240 and TLA0015-11 antibodies are shown in FIG. 22, the half-life T1/2 of the TLA0015-11 antibody in hFcRn mice is 18.46 days, and the concentration of the control antibody RVT-3101 in the serum samples on day 14 and day 21 is below the lower limit of detection. In addition, the results of the same batch of RVT-3101 and TLA0238-5 antibodies are shown in FIG. 23, the half-life T1/2 time of TLA0238-5 antibody in hFcRn mice is 19.12 days, and the concentration of control antibody RVT-3101 in serum samples on day 14 and day 21 is below the lower limit of detection.

TABLE 24
PK blood collection time point

| Mice | Point-in-time | | | | | | | | | | | | |
|---|---|---|---|---|---|---|---|---|---|---|---|---|---|
| 1 | 5 min | | 1 h | | 6 hours | | 48 h | | 96 h | | 7 d | | 21 d |
| 2 | | | | | | | | | | | | | |
| 3 | | | | | | | | | | | | | |
| 4 | | | | | | | | | | | | | |
| 5 | | | | | | | | | | | | | |
| 6 | | 30 min | | 2 hours | | 24 h | | 72 h | | 120 h | | 14 d | |
| 7 | | | | | | | | | | | | | |
| 8 | | | | | | | | | | | | | |
| 9 | | | | | | | | | | | | | |
| 10 | | | | | | | | | | | | | |

### Example 21 Efficacy of Anti-TL1A Antibody in DSS-Induced hTL1A Transgenic Mouse Enteritis Model

In this experiment, TL1A humanized transgenic mice were used to investigate the efficacy of anti-TL1A antibodies in a DSS-induced mouse model of enteritis. TL1A humanized transgenic mouse B-hTL1A mice (Catalog: 111997) was purchased from Biocytogen (Beijing) Pharmaceutical Technology Co., Ltd., the humanized transgenic mouse knocked out the mouse TL1A gene on the C57BL/6 strain mouse, and knocked in the human TL1A gene, the transgenic mouse only expressed the human TL1A protein, not the mouse TL1A protein. After dissolving DSS in drinking water, the intestinal epithelial cells of mice were destroyed after drinking water containing DSS, and non-specific immune cells released cytokines, which eventually led to the destruction of the integrity of the mucosal barrier, and the mice showed obvious weight loss, loose stool, blood in the stool and granulocyte infiltration, which were extremely similar to human ulcerative colitis in terms of clinical symptoms and pathological features. A DSS-induced mouse enteritis model was used to verify the efficacy of anti-TL1A antibody in enteritis.

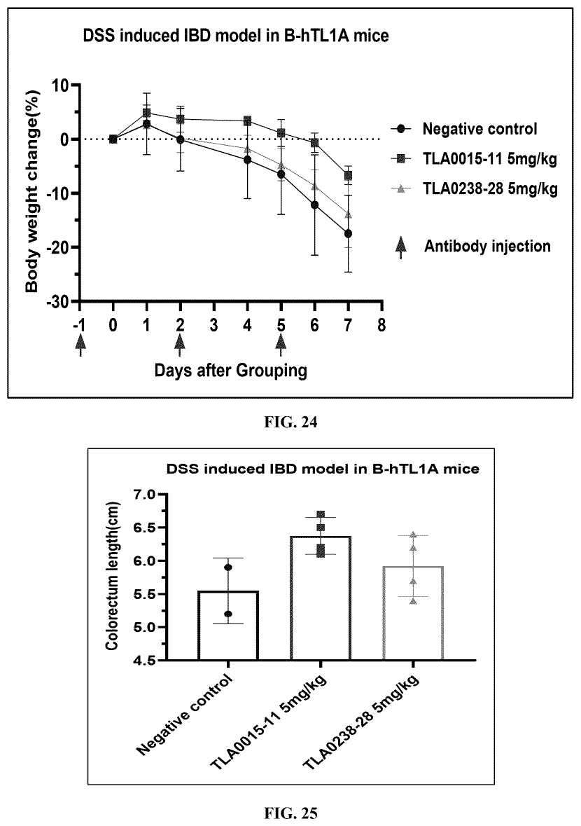

Ten B-hTL1A transgenic mice were randomly divided into three groups, including two mice in the negative control group, four mice in each of the TLA0015-11 monoclonal antibody group and the TLA0238-28 monoclonal antibody group. The drug was injected into the tail vein on day 0, day 3 and day 6, respectively, at a dose of 5 mg/kg: Mice were given drinking water dissolved in 4% DSS from day 2 to day 8. During the experiment, the weight of the experimental animals was measured and recorded every day, and the daily activities of the animals were observed. Experimental animal fecal traits are scored daily (0=normal, 1=moist/sticky, 2=fluffy, 3=liquid). At the end of the experiment on the 8th day, all experimental animals were euthanized by suffocation with excess carbon dioxide, after which the abdominal cavity was cut, the colon was removed, and the length of the colon was recorded. The results of weight change are shown in FIG. 24, and it can be seen from the negative control group that the weight loss of mice after DSS-induced enteritis was significant, while the weight loss of mice in the TLA0015-11 monoclonal antibody administration group was significantly less than that of the negative control. The results of the change in colorectal length are shown in FIG. 25, and it can be seen from the negative control group that the colorectal length of mice was significantly shortened after DSS-induced enteritis, while the colorectal length of mice in the TLA0015-11 monoclonal antibody administration group was significantly restored. In summary, it can be seen that TLA0015-11 antibody has a good therapeutic effect in a DSS-induced enteritis model.

To explore the relationship between the efficacy of TLA0015-11 antibody and the dose in the DSS-induced enteritis model, three dose groups were designed, namely, 1 mg/kg, 3 mg/kg and 10 mg/kg, and the specific groupings are shown in Table 25, and the experiments were carried out as described above.

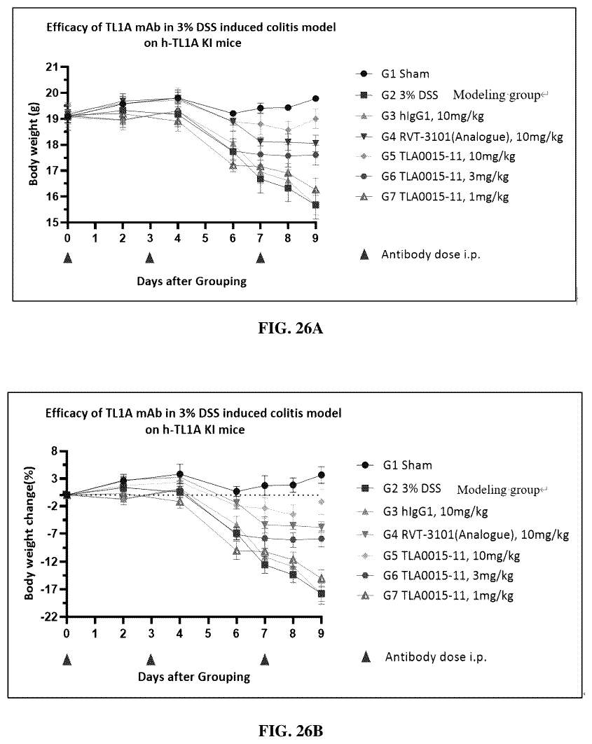

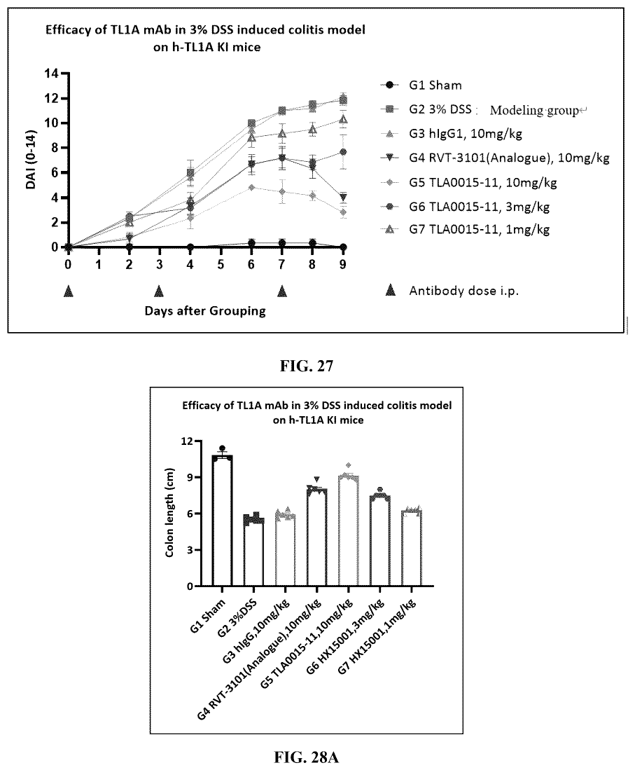

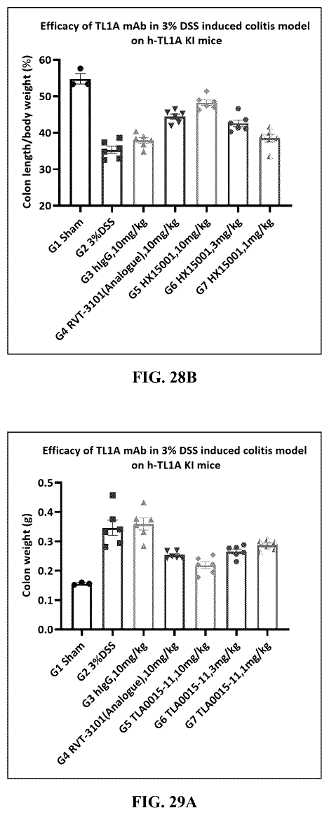

Experimental results: the results of the body weight changes of mice in each group are shown in FIG. 26A and FIG. 26B, and it can be seen that the body weights of mice in the modeling group and the IgG-negative control group were significantly decreased, and TLA0015-11 monoclonal antibody had a very good pharmacodynamic activity at the doses of 3 mg/kg and 10 mg/kg, and TLA0015-11 monoclonal antibody had a weak pharmacodynamic activity in the case of 1 mg/kg: the control antibodyRVT-3101 also had good pharmacodynamic activity. The results of daily scoring are shown in FIG. 27, in which the high dose group of TLA0015-11 antibody was scored better than the low dose group, and the potency was better than the control antibody RVT-3101 at the dose of 10 mg/kg. At the end of the experiment, all mice survived, and there was no difference in the survival curves. After the mice were dissected, the results of colorectal length are shown in FIGS. 28A and 28B, and the results of colorectal weight are shown in FIGS. 29A and 29B. The efficacy was seen in both the TLA0015-11 and RVT-3101 dosing groups, with a clear efficacy-dose relationship for TLA0015-11, with the higher the dose, the better the efficacy. In conclusion, TLA0015-11 antibody has a good in vivo pharmacodynamic activity in the DSS-induced murine enteritis model, and the pharmacodynamic effect is dose-related.

TABLE 25
Grouping of mice and details of drug administration
(DSS-induced enteritis model)

| | | Number | | | |
|---|---|---|---|---|---|
| | | of | | | |
| Group | Animal | Animals | Modeling | Treatment | Dosing |
| G1 | hTL1A | 3 | — | — | — |
| G2 | mice | 6 | DSS | — | — |
| G3 | | 6 | | hIgG1 | 10 mg/kg, 3 dose |
| G4 | | 6 | | RVT-3101 | 10 mg/kg, 3 dose |
| | | | | (Analogue) | |
| G5 | | 6 | | TLA0015-11 | 10 mg/kg, 3 dose |
| G6 | | 6 | | TLA0015-11 | 3 mg/kg, 3 dose |
| G7 | | 6 | | TLA0015-11 | 1 mg/kg, 3 dose |

### Example 22 Efficacy of Anti-TL1A Antibody in TNBS-Induced Enteritis Model in hTL1A Transgenic Mice

In this experiment, hTL1A transgenic mice were used to study the in vivo pharmacodynamic activity of anti-TL1A antibodies in a TNBS-induced model of enterocolitis. TNBS-treated mice establish a preclinical model that mimics clinical Crohn's disease (CD), which produces a Th1-mediated immune response characterized by infiltration of CD4+ T cells, neutrophils, and macrophages that develops laterally progressive inflammation that leading to colitis. Therefore, the TNBS-induced enteritis model can be used to evaluate the in vivo pharmacodynamic activity of antibody drugs.

Forty-four male hTL1A transgenic mice were randomly divided into six groups based on body weight, including four mice in the G1 negative control group, eight mice in each of the G2 modeling group, G3 hIgG-negative control group, G4 RVT-3101 (analog) drug group, G5 MK-7240 (analog) drug group, and G6 TLA0015-11 administration group, and the detailed grouping of the mice is shown in Table 26. day 1 began intraperitoneal administration of the drug at three-day intervals for a total of three administrations. All animals were fasted on Day 0:100 μl of 60% ethanol was given (i.r.) rectally to the G1 negative control group on Day 1, and 100 μl of 2% TNBS was given (i.r.) rectally to the G2 to G6 groups and feed was resumed to all animals to induce acute enterocolitis. During the experiment, the body weights of the experimental animals were measured and recorded daily, and the daily activities of the animals were observed: the fecal traits of the experimental animals were scored daily (0=normal, 1=moist/sticky, 2=fluffy, 3=liquid). At the end of the experiment on day 9, all experimental animals were euthanized using excess carbon dioxide asphyxiation after which the abdominal cavity was cut open, the colon was removed, and the length of the colon was recorded.

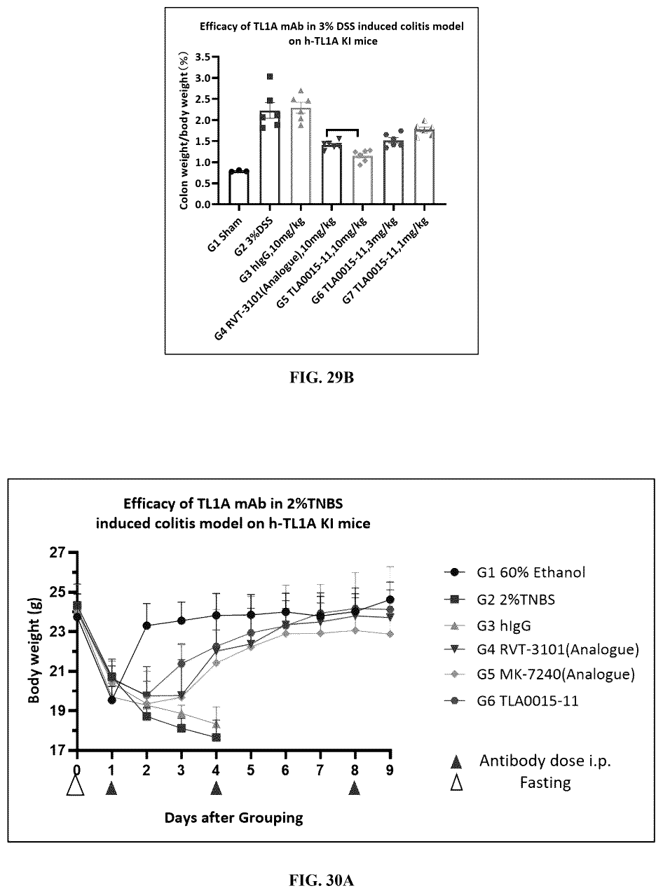

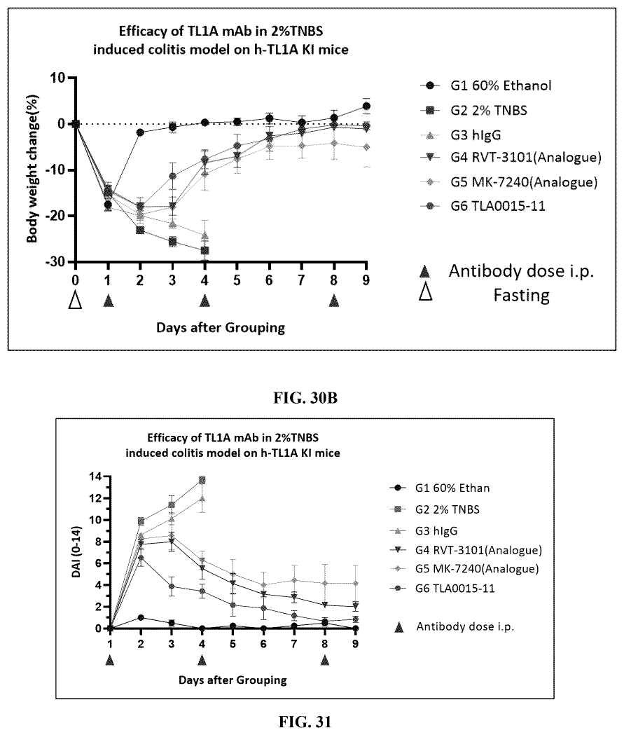

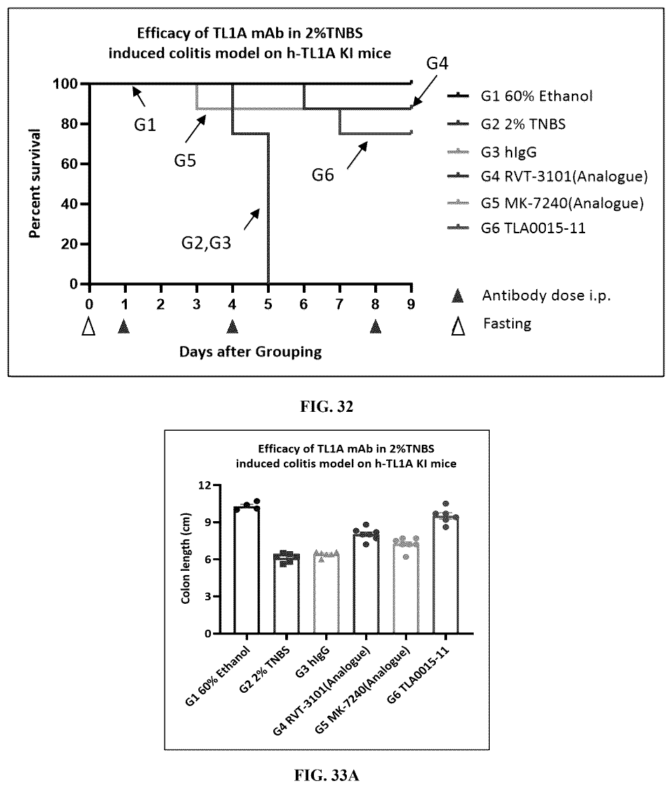

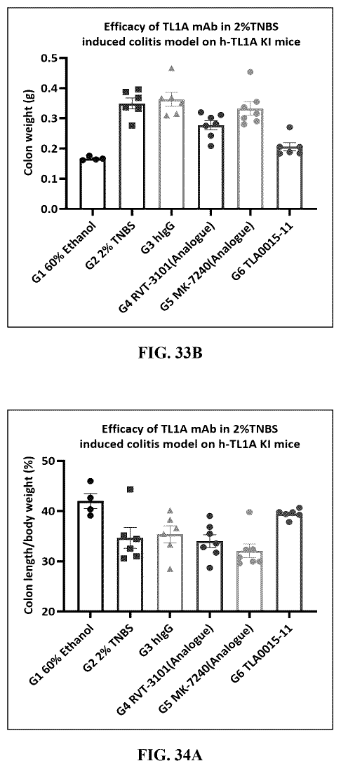

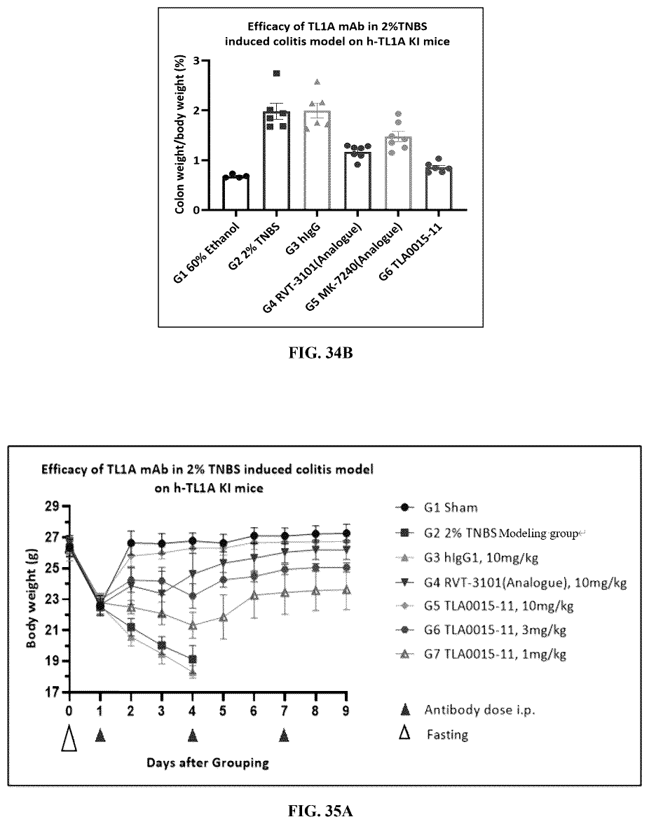

At the end of the experiment, the results were statistically analyzed, in which the results of mice weight changes are shown in FIG. 30A and FIG. 30B, and the weight regain of mice can be seen in the RVT-3101 (analog) drug group, the MK-7240 (analog) drug group, and the TLA0015-11 administration group when compared with the G2 modeling group. From the results of daily activity scores (FIG. 31), the scores of RVT-3101 (analog) drug group, MK-7240 (analog) drug group and TLA0015-11 administration group started to decrease after day 2. The results of the animal survival curves are shown in FIG. 32, in which all mice in the modeling group and hIgG-negative control group died at day 5. At the end of day 9, some mice in the three TL1A antibody-administered groups also died. Colorectal length and weight were measured after dissection, and the results of colorectal length in mice are shown in FIGS. 33A and 33B, and the results of colorectal weight are shown in FIGS. 34A and 34B. Significant shortening of colorectal length and increase in colorectal weight were seen in the G2 modeling group. efficacy of the drug could be seen in RVT-3101, MK-7240 and TLA0015-11.

TABLE 26
Grouping of mice and details of drug administration
(TNBS-induced enteritis model)

| | | Number | | | |
|---|---|---|---|---|---|
| | | of | | | |
| Group | Animal | Animals | Modeling | Treatment | Dosing |
| G1 | hTL1A | 4 | — | — | — |
| G2 | mice | 8 | TNBS | — | — |
| G3 | | 8 | | hIgG1 | 10 mg/kg, 3 dose |
| G4 | | 8 | | RVT-3101 | 10 mg/kg, 3 dose |
| | | | | (Analogue) | |
| G5 | | 8 | | MK-7240 | 10 mg/kg, 3 dose |
| | | | | (Analogue) | |
| G6 | | 8 | | TLA0015-11 | 10 mg/kg, 3 dose |

To explore the relationship between the efficacy of TLA0015-11 antibody and the dose in the TNBS-induced enteritis model, three dose groups were designed, which were 1 mg/kg, 3 mg/kg and 10 mg/kg, and the specific groupings are shown in Table 27, and the experiments were carried out as described above.

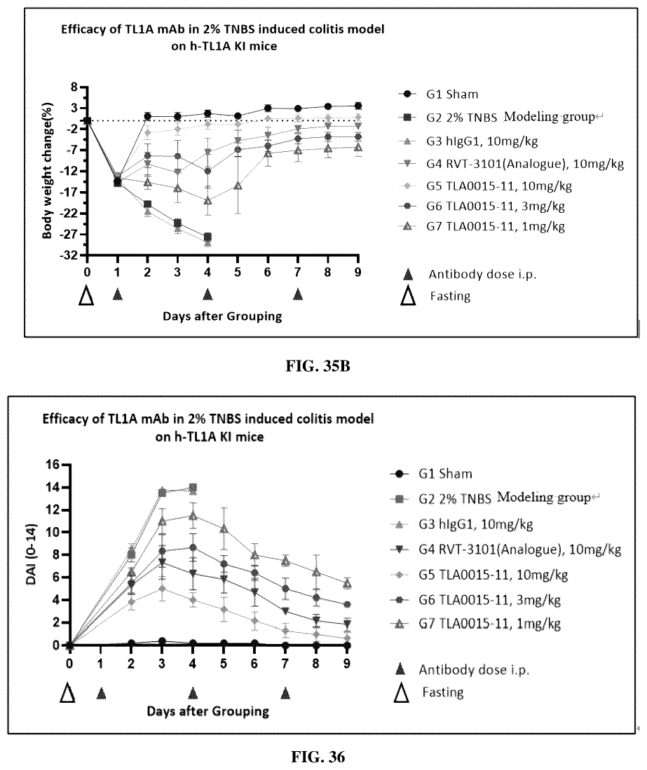

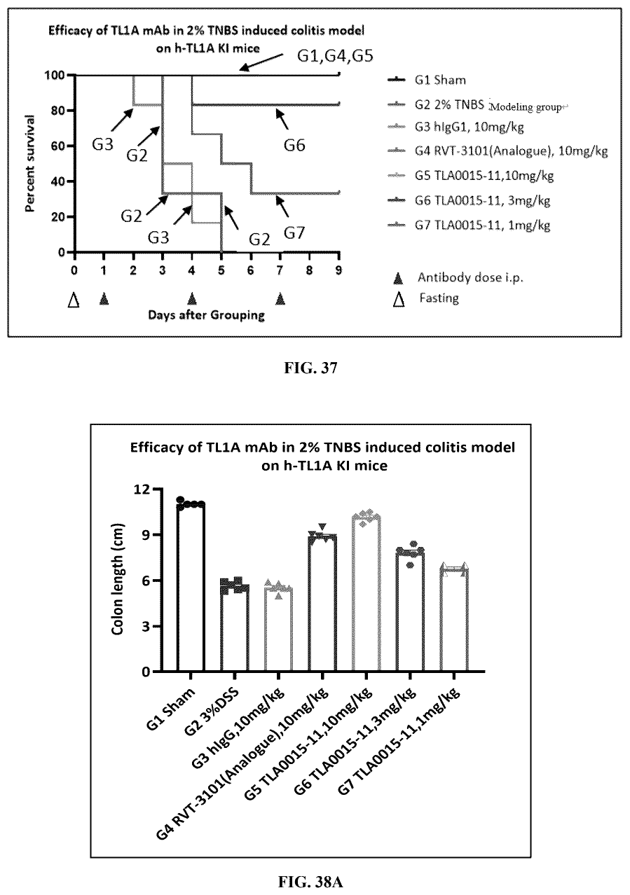

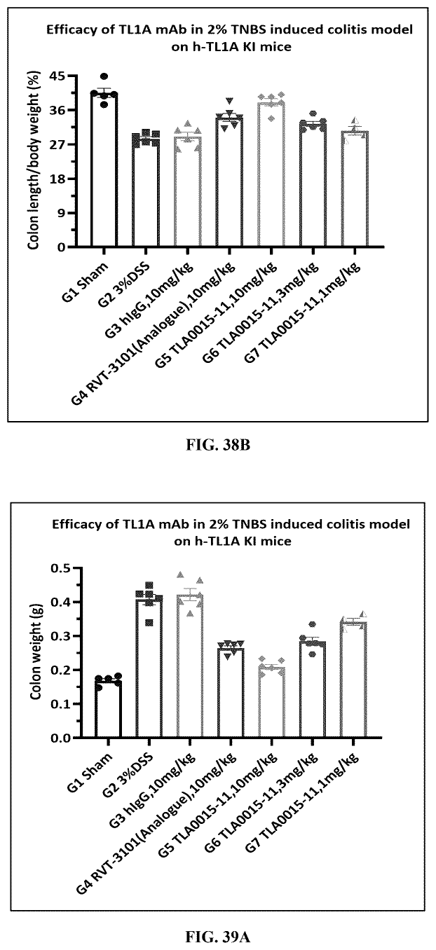

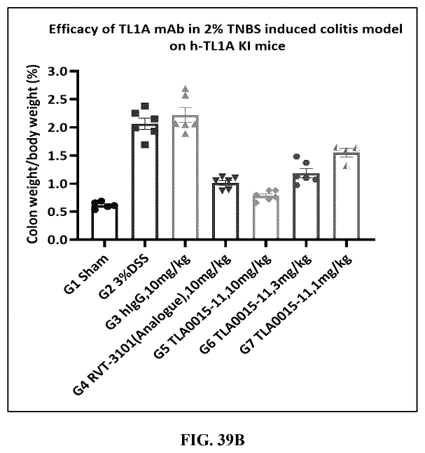

Experimental results: the results of the body weight changes of mice in each group are shown in FIGS. 35A and 35B, and it can be seen that the body weights of mice in both the modeling group and the IgG-negative control group were significantly decreased, and TLA0015-11 monoclonal antibody had a very good pharmacodynamic activity at the doses of 3 mg/kg and 10 mg/kg, and TLA0015-11 monoclonal antibody had a weak pharmacodynamic activity in the case of 1 mg/kg: the control antibodyRVT-3101 also had good pharmacodynamic activity. The results of daily scoring are shown in FIG. 36, in which the TLA0015-11 antibody high-dose group scored better than the low-dose group and was more potent than the control antibody RVT-3101 at a dose of 10 mg/kg. The results of the animal survival curves are shown in FIG. 37, in which all the mice in the modeling group and the hIgG-negative control group were dead at the end of day 5. At the end of day 9, no mice died at the dose of 10 mg/kg for RVT-3101 and TLA0015-11; one mouse died at the dose of 3 mg/kg for TLA0015-11; and four mice died at the dose of 1 mg/kg. After the mice were dissected, the results of colorectal length are shown in FIGS. 38A and 38B, and the results of colorectal weight are shown in FIGS. 39A and 39B. The efficacy of the drug was seen in both the TLA0015-11 and RVT-3101 dosing groups, with a clear dose-dependent efficacy of TLA0015-11, with the higher the dose, the better the efficacy. In conclusion, TLA0015-11 antibody has good in vivo pharmacodynamic activity in TNBS-induced murine enteritis model, and the pharmacodynamic effect is dose-related.

TABLE 27
Grouping of mice and details of drug administration
(TNBS-induced enteritis model)

| | | Number | | | |
|---|---|---|---|---|---|
| | | of | | | |
| Group | Animal | Animals | Modeling | Treatment | Dosing |
| G1 | hTL1A | 3 | — | — | — |
| G2 | mice | 6 | TNBS | — | — |
| G3 | | 6 | | hIgG1 | 10 mg/kg, 3 dose |
| G4 | | 6 | | RVT-3101 | 10 mg/kg, 3 dose |
| | | | | (Analogue) | |
| G5 | | 6 | | TLA0015-11 | 10 mg/kg, 3 dose |
| G6 | | 6 | | TLA0015-11 | 3 mg/kg, 3 dose |
| G7 | | 6 | | TLA0015-11 | 1 mg/kg, 3 dose |

In summary, the sequences used in the present application are shown in the table below, wherein [ ] represents that the position can be arbitrarily replaced by the amino acids shown in [ ].

| c | LABEL | SEQ |
|---|---|---|
| 1 | TLA0015- | [STY]YGVD |
| | HCDR1 | |
| 2 | TLA0015- | SYGVD |
| | HCDR1-1 | |
| 3 | TLA0015 | YYGVD |
| | HCDR1-2 | |
| 4 | TLA0015- | TYGVD |
| | HCDR1-3 | |
| 5 | TLA0015- | [RV]IWGFG[GK]T[DHN]Y[NQ][APS]ALKS |
| | HCDR2 | |
| 6 | TLA0015- | VIWGFGGTNYNSALKS |
| | HCDR2-1 | |
| 7 | TLA0015- | VIWGFGGTNYQSALKS |
| | HCDR2-2 | |
| 8 | TLA0015- | VIWGFGGTNYNAALKS |
| | HCDR2-3 | |
| 9 | TLA0015- | VIWGFGGTNYNPALKS |
| | HCDR2-4 | |
| 10 | TLA0015- | VIWGFGGTHYNSALKS |
| | HCDR2-5 | |
| 11 | TLA0015- | RIWGFGGTNYNSALKS |
| | HCDR2-6 | |
| 12 | TLA0015- | VIWGFGKTNYNSALKS |
| | HCDR2-7 | |
| 13 | TLA0015- | VIWGFGGTDYNSALKS |
| | HCDR2-8 | |
| 14 | TLA0015- | G[DLN][FY]DAMDY |
| | HCDR3 | |
| 15 | TLA0015- | GNFDAMDY |
| | HCDR3-1 | |
| 16 | TLA0015- | GNYDAMDY |
| | HCDR3-2 | |
| 17 | TLA0015- | GDFDAMDY |
| | HCDR3-3 | |
| 18 | TLA0015- | GLEDAMDY |
| | HCDR3-4 | |
| 19 | TLA0015- | E[IV]QL[LV]ESGGGL[IV]QPGGSLR[IL]SCAVSGF[DS]L[IKS] |
| | HFR1 | |
| 20 | TLA0015- | EIQLLESGGGLVQPGGSLRLSCAVSGFSLS |
| | HFR1-1 | |
| 21 | TLA0015- | EIQLLESGGGLVQPGGSLRISCAVSGFSLS |
| | HFR1-2 | |
| 22 | TLA0015- | EIQLVESGGGLVQPGGSLRISCAVSGFSLS |
| | HFR1-3 | |
| 23 | TLA0015- | EVQLLESGGGLVQPGGSLRLSCAVSGFSLS |
| | HFR1-4 | |
| 24 | TLA0015- | EIQLVESGGGLIQPGGSLRLSCAVSGFSLS |
| | HFR1-5 | |
| 25 | TLA0015- | EIQLVESGGGLIQPGGSLRISCAVSGFSLS |
| | HFR1-6 | |
| 26 | TLA0015- | EIQLVESGGGLIQPGGSLRISCAVSGFSLI |
| | HFR1-7 | |
| 27 | TLA0015- | EIQLVESGGGLIQPGGSLRISCAVSGFSLK |
| | HFR1-8 | |
| 28 | TLA0015- | EIQLVESGGGLIQPGGSLRISCAVSGFDLS |
| | HFR1-9 | |
| 29 | TLA0015- | WVRQ[AS]PGKGLEW[LV]G |
| | HFR2 | |
| 30 | TLA0015- | WVRQAPGKGLEWVG |
| | HFR2- | |
| | 1/TLA-0238- | |
| | HFR2-3 | |
| 31 | TLA0015 | WVRQAPGKGLEWLG |
| | HFR2-2 | |
| 32 | TLA0015- | WVRQSPGKGLEWLG |
| | HFR2-3 | |
| 33 | TLA0015- | R[FL]TIS[AKRV]DNSKNT[LV]YLQMNSLRAEDTAVYYCA[RS] |
| | HFR3 | |
| 34 | TLA0015- | RLTISRDNSKNTLYLQMNSLRAEDTAVYYCAS |
| | HFR3-1 | |
| 35 | TLA0015- | RLTISKDNSKNTVYLQMNSLRAEDTAVYYCAS |
| | HFR3-2 | |
| 36 | TLA0015- | RFTISRDNSKNTVYLQMNSLRAEDTAVYYCAS |
| | HFR3-3 | |
| 37 | TLA0015- | RLTISRDNSKNTVYLQMNSLRAEDTAVYYCAS |
| | HFR3-4 | |
| 38 | TLA0015- | RLTISRDNSKNTVYLQMNSLRAEDTAVYYCAR |
| | HFR3-5 | |
| 39 | TLA0015- | RLTISVDNSKNTVYLQMNSLRAEDTAVYYCAS |
| | HFR3-6 | |
| 40 | TLA0015- | RLTISADNSKNTVYLQMNSLRAEDTAVYYCAS |
| | HFR3-7 | |
| 41 | TLA0015- | WGQGTLVTVSS |
| | HFR4- | |
| | 1/TLA-0238- | |
| | HFR4-2 | |
| 42 | TLA0015- | RASQDISNYLN |
| | LCDR1-1 | |
| 43 | TL0015- | YTSRLHS |
| | LCDR2-1 | |
| 44 | TL0015- | QQGNTLPFT |
| | LCDR3-1 | |
| 45 | TL0015- | DIQMTQ[ST][PT]SSLSASVGDRVTI[TS]C |
| | LFR1 | |
| 46 | TL0015- | DIQMTQTTSSLSASLGDRVTISC |
| | LFR1-1 | |
| 47 | TL0015- | DIQMTQSPSSLSASVGDRVTITC |
| | LFR1- | |
| | 2/TLA0241- | |
| | LFR1-1 | |
| 48 | TL0015- | WYQQKPGK[AV][PV]KLLIY |
| | LFR2 | |
| 49 | TL0015- | WYQQKPDGTVKLLIY |
| | LFR2-1 | |
| 50 | TL0015- | WYQQKPGKVVKLLIY |
| | LFR2-2 | |
| 51 | TL0015- | WYQQKPGKAVKLLIY |
| | LFR2-3 | |
| 52 | TL0015- | WYQQKPGKAPKLLIY |
| | LFR2- | |
| | 4/TLA0241- | |
| | LFR2-1 | |
| 53 | TL0015- | GVPSRFSGSGSGTD[FY]TLTISSLQPED[FV]ATY[FY]C |
| | LFR3 | |
| 54 | TL0015- | GVPSRFSGSGSGTDYSLTISNLEQEDIATYFC |
| | LFR3-1 | |
| 55 | TL0015 | GVPSRFSGSGSGTDYTLTISSLQPEDVATYFC |
| | LFR3-2 | |
| 56 | TL0015- | GVPSRFSGSGSGTDYTLTISSLQPEDFATYFC |
| | LFR3-3 | |
| 57 | TL0015- | GVPSRFSGSGSGTDFTLTISSLQPEDFATYYC |
| | LFR3- | |
| | 4/TLA0238- | |
| | LFR3- | |
| | 4/TLA0241- | |
| | LFR3-2 | |
| 58 | TL0015- | FG[PQ]GTKLEIK |
| | LFR4 | |
| 59 | TL0015- | FGSGTKLEIK |
| | LFR4-1 | |
| 60 | TL0015- | FGPGTKLEIK |
| | LFR4-2 | |
| 61 | TL0015- | FGQGTKLEIK |
| | LFR4- | |
| | 3/TLA0238- | |
| | LFR4-3 | |
| 62 | TLA0015- | QIQLKESGPGLVAPSQSLSITCTVSGFSLSSYGVDWVRQSPGKGLEWLGVIWGFGGTNYN |
| | VH-1 | SALKSRLSISKDNSKSQVFLKMNSLQSDDSAIYYCASGNFDAMDYWGQGTSVTVSS |
| 63 | TLA0015- | EIQLLESGGGLVQPGGSLRLSCAVSGFSLSSYGVDWVRQAPGKGLEWVGVIWGFGGTN |
| | VH-2 | YNSALKSRLTISRDNSKNTLYLQMNSLRAEDTAVYYCASGNFDAMDYWGQGTLVTVSS |
| 64 | TLA0015- | EIQLLESGGGLVQPGGSLRISCAVSGFSLSSYGVDWVRQAPGKGLEWLGVIWGFGGTNY |
| | VH-3 | NSALKSRLTISKDNSKNTVYLQMNSLRAEDTAVYYCASGNFDAMDYWGQGTLVTVSS |
| 65 | TLA0015- | EIQLVESGGGLVQPGGSLRISCAVSGFSLSSYGVDWVRQSPGKGLEWLGVIWGFGGTNY |
| | VH-4 | NSALKSRLTISKDNSKNTVYLQMNSLRAEDTAVYYCASGNFDAMDYWGQGTLVTVSS |
| 66 | TLA0015- | EVQLLESGGGLVQPGGSLRLSCAVSGFSLSSYGVDWVRQAPGKGLEWLGVIWGFGGTN |
| | VH-5 | YNSALKSRFTISRDNSKNTVYLQMNSLRAEDTAVYYCASGNFDAMDYWGQGTLVTVSS |
| 67 | TLA0015- | EVQLLESGGGLVQPGGSLRLSCAVSGFSLSSYGVDWVRQAPGKGLEWLGVIWGFGGTN |
| | VH-6 | YNSALKSRLTISRDNSKNTVYLQMNSLRAEDTAVYYCASGNFDAMDYWGQGTLVTVSS |
| 68 | TLA0015 | EVQLLESGGGLVQPGGSLRLSCAVSGFSLSSYGVDWVRQAPGKGLEWLGVIWGFGGTN |
| | VH-7 | YNSALKSRLTISKDNSKNTVYLQMNSLRAEDTAVYYCASGNFDAMDYWGQGTLVTVSS |
| 69 | TLA0015- | EIQLVESGGGLIQPGGSLRLSCAVSGFSLSSYGVDWVRQAPGKGLEWLGVIWGFGGTNY |
| | VH-8 | NSALKSRLTISRDNSKNTVYLQMNSLRAEDTAVYYCARGNFDAMDYWGQGTLVTVSS |
| 70 | TLA0015- | EIQLVESGGGLIQPGGSLRISCAVSGFSLSSYGVDWVRQAPGKGLEWLGVIWGFGGTNY |
| | VH-9 | NSALKSRLTISKDNSKNTVYLQMNSLRAEDTAVYYCASGNFDAMDYWGQGTLVTVSS |
| 71 | TLA0015- | EIQLVESGGGLIQPGGSLRISCAVSGFSLSSYGVDWVRQAPGKGLEWLGVIWGFGGTNY |
| | VH-10 | NSALKSRLTISRDNSKNTVYLQMNSLRAEDTAVYYCASGNFDAMDYWGQGTLVTVSS |
| 72 | TLA0015- | EIQLVESGGGLIQPGGSLRISCAVSGFSLSSYGVDWVRQAPGKGLEWLGVIWGFGGTNY |
| | VH-11 | NSALKSRLTISVDNSKNTVYLQMNSLRAEDTAVYYCASGNFDAMDYWGQGTLVTVSS |
| 73 | TLA0015- | EIQLVESGGGLIQPGGSLRISCAVSGFSLSSYGVDWVRQAPGKGLEWLGVIWGFGGTNY |
| | VH-12 | QSALKSRLTISKDNSKNTVYLQMNSLRAEDTAVYYCASGNFDAMDYWGQGTLVTVSS |
| 74 | TLA0015- | EIQLVESGGGLIQPGGSLRISCAVSGFSLSSYGVDWVRQAPGKGLEWLGVIWGFGGTNY |
| | VH-13 | NAALKSRLTISKDNSKNTVYLQMNSLRAEDTAVYYCASGNFDAMDYWGQGTLVTVSS |
| 75 | TLA0015- | EIQLVESGGGLIQPGGSLRISCAVSGFSLSSYGVDWVRQAPGKGLEWLGVIWGFGGTNY |
| | VH-14 | NSALKSRLTISADNSKNTVYLQMNSLRAEDTAVYYCASGNFDAMDYWGQGTLVTVSS |
| 76 | TLA0015- | EIQLVESGGGLIQPGGSLRISCAVSGFSLSYYGVDWVRQAPGKGLEWLGVIWGFGGTNY |
| | VH-15 | NSALKSRLTISKDNSKNTVYLQMNSLRAEDTAVYYCASGNFDAMDYWGQGTLVTVSS |
| 77 | TLA0015- | EIQLVESGGGLIQPGGSLRISCAVSGFSLSSYGVDWVRQAPGKGLEWLGVIWGFGGTNY |
| | VH-16 | NSALKSRLTISKDNSKNTVYLQMNSLRAEDTAVYYCASGNYDAMDYWGQGTLVTVSS |
| 78 | TLA0015- | EIQLVESGGGLIQPGGSLRISCAVSGFSLSSYGVDWVRQAPGKGLEWLGVIWGFGGTNY |
| | VH-17 | NSALKSRLTISKDNSKNTVYLQMNSLRAEDTAVYYCASGDFDAMDYWGQGTLVTVSS |
| 79 | TLA0015- | EIQLVESGGGLIQPGGSLRISCAVSGFSLSSYGVDWVRQAPGKGLEWLGVIWGFGGTNY |
| | VH-18 | NSALKSRLTISKDNSKNTVYLQMNSLRAEDTAVYYCASGLFDAMDYWGQGTLVTVSS |
| 80 | TLA0015- | EIQLVESGGGLIQPGGSLRISCAVSGFSLSTYGVDWVRQAPGKGLEWLGVIWGFGGTNY |
| | VH-19 | NSALKSRLTISKDNSKNTVYLQMNSLRAEDTAVYYCASGNFDAMDYWGQGTLVTVSS |
| 81 | TLA0015- | EIQLVESGGGLIQPGGSLRISCAVSGFSLISYGVDWVRQAPGKGLEWLGVIWGFGGTNYN |
| | VH-20 | SALKSRLTISKDNSKNTVYLQMNSLRAEDTAVYYCASGNFDAMDYWGQGTLVTVSS |
| 82 | TLA0015- | EIQLVESGGGLIQPGGSLRISCAVSGFSLSSYGVDWVRQAPGKGLEWLGVIWGFGGTNY |
| | VH-21 | NPALKSRLTISKDNSKNTVYLQMNSLRAEDTAVYYCASGNFDAMDYWGQGTLVTVSS |
| 83 | TLA0015- | EIQLVESGGGLIQPGGSLRISCAVSGFSLKSYGVDWVRQAPGKGLEWLGVIWGFGGTNY |
| | VH-22 | NSALKSRLTISKDNSKNTVYLQMNSLRAEDTAVYYCASGNFDAMDYWGQGTLVTVSS |
| 84 | TLA0015- | EIQLVESGGGLIQPGGSLRISCAVSGFSLSSYGVDWVRQAPGKGLEWLGVIWGFGGTHY |
| | VH-23 | NSALKSRLTISKDNSKNTVYLQMNSLRAEDTAVYYCASGNFDAMDYWGQGTLVTVSS |
| 85 | TLA0015- | EIQLVESGGGLIQPGGSLRISCAVSGFSLSSYGVDWVRQAPGKGLEWLGRIWGFGGTNY |
| | VH-24 | NSALKSRLTISKDNSKNTVYLQMNSLRAEDTAVYYCASGNFDAMDYWGQGTLVTVSS |
| 86 | TLA0015- | EIQLVESGGGLIQPGGSLRISCAVSGFDLSSYGVDWVRQAPGKGLEWLGVIWGFGGTNY |
| | VH-25 | NSALKSRLTISKDNSKNTVYLQMNSLRAEDTAVYYCASGNFDAMDYWGQGTLVTVSS |
| 87 | TLA0015- | EIQLVESGGGLIQPGGSLRISCAVSGFSLSSYGVDWVRQAPGKGLEWLGVIWGFGKTNY |
| | VH-26 | NSALKSRLTISKDNSKNTVYLQMNSLRAEDTAVYYCASGNFDAMDYWGQGTLVTVSS |
| 88 | TLA0015- | EIQLVESGGGLIQPGGSLRISCAVSGFSLSSYGVDWVRQAPGKGLEWLGVIWGFGGTDY |
| | VH-27 | NSALKSRLTISKDNSKNTVYLQMNSLRAEDTAVYYCASGNFDAMDYWGQGTLVTVSS |
| 89 | TLA0015- | EIQLVESGGGLIQPGGSLRISCAVSGFSLSYYGVDWVRQAPGKGLEWLGVIWGFGGTNY |
| | VH-28 | NSALKSRLTISKDNSKNTVYLQMNSLRAEDTAVYYCASGNYDAMDYWGQGTLVTVSS |
| 90 | TLA0015- | EIQLVESGGGLIQPGGSLRISCAVSGFSLSYYGVDWVRQAPGKGLEWLGVIWGFGGTNY |
| | VH-29 | NSALKSRLTISKDNSKNTVYLQMNSLRAEDTAVYYCASGDFDAMDYWGQGTLVTVSS |
| 91 | TLA0015- | EIQLVESGGGLIQPGGSLRISCAVSGFSLSYYGVDWVRQAPGKGLEWLGVIWGFGGTNY |
| | VH-30 | NPALKSRLTISKDNSKNTVYLQMNSLRAEDTAVYYCASGNFDAMDYWGQGTLVTVSS |
| 92 | TLA0015- | EIQLVESGGGLIQPGGSLRISCAVSGFSLSYYGVDWVRQAPGKGLEWLGVIWGFGGTHY |
| | VH-31 | NSALKSRLTISKDNSKNTVYLQMNSLRAEDTAVYYCASGNFDAMDYWGQGTLVTVSS |
| 93 | TLA0015- | EIQLVESGGGLIQPGGSLRISCAVSGFSLSYYGVDWVRQAPGKGLEWLGRIWGFGGTNY |
| | VH-32 | NSALKSRLTISKDNSKNTVYLQMNSLRAEDTAVYYCASGNFDAMDYWGQGTLVTVSS |
| 94 | TLA0015- | EIQLVESGGGLIQPGGSLRISCAVSGFDLSYYGVDWVRQAPGKGLEWLGVIWGFGGTNY |
| | VH-33 | NSALKSRLTISKDNSKNTVYLQMNSLRAEDTAVYYCASGNFDAMDYWGQGTLVTVSS |
| 95 | TLA0015- | EIQLVESGGGLIQPGGSLRISCAVSGFSLISYGVDWVRQAPGKGLEWLGVIWGFGGTNYN |
| | VH-34 | SALKSRLTISKDNSKNTVYLQMNSLRAEDTAVYYCASGNYDAMDYWGQGTLVTVSS |
| 96 | TLA0015- | EIQLVESGGGLIQPGGSLRISCAVSGFSLSSYGVDWVRQAPGKGLEWLGVIWGFGGTNY |
| | VH-35 | NPALKSRLTISKDNSKNTVYLQMNSLRAEDTAVYYCASGNYDAMDYWGQGTLVTVSS |
| 97 | TLA0015- | EIQLVESGGGLIQPGGSLRISCAVSGFSLSSYGVDWVRQAPGKGLEWLGVIWGFGGTHY |
| | VH-36 | NSALKSRLTISKDNSKNTVYLQMNSLRAEDTAVYYCASGNYDAMDYWGQGTLVTVSS |
| 98 | TLA0015- | EIQLVESGGGLIQPGGSLRISCAVSGFSLSSYGVDWVRQAPGKGLEWLGRIWGFGGTNY |
| | VH-37 | NSALKSRLTISKDNSKNTVYLQMNSLRAEDTAVYYCASGNYDAMDYWGQGTLVTVSS |
| 99 | TLA0015- | EIQLVESGGGLIQPGGSLRISCAVSGFSLSSYGVDWVRQAPGKGLEWLGVIWGFGKTNY |
| | VH-38 | NSALKSRLTISKDNSKNTVYLQMNSLRAEDTAVYYCASGNYDAMDYWGQGTLVTVSS |
| 100 | TLA0015- | EIQLVESGGGLIQPGGSLRISCAVSGFSLISYGVDWVRQAPGKGLEWLGVIWGFGGTNYN |
| | VH-39 | SALKSRLTISKDNSKNTVYLQMNSLRAEDTAVYYCASGDFDAMDYWGQGTLVTVSS |
| 101 | TLA0015- | EIQLVESGGGLIQPGGSLRISCAVSGFSLSSYGVDWVRQAPGKGLEWLGVIWGFGGTHY |
| | VH-40 | NSALKSRLTISKDNSKNTVYLQMNSLRAEDTAVYYCASGDFDAMDYWGQGTLVTVSS |
| 102 | TLA0015- | DIQMTQTTSSLSASLGDRVTISCRASQDISNYLNWYQQKPDGTVKLLIYYTSRLHSGVPS |
| | VL-1 | RFSGSGSGTDYSLTISNLEQEDIATYFCQQGNTLPFTFGSGTKLEIK |
| 103 | TLA0015- | DIQMTQSPSSLSASVGDRVTITCRASQDISNYLNWYQQKPGKVVKLLIYYTSRLHSGVPS |
| | VL-2 | RFSGSGSGTDYTLTISSLQPEDVATYFCQQGNTLPFTFGPGTKLEIK |
| 104 | TLA0015- | DIQMTQSPSSLSASVGDRVTITCRASQDISNYLNWYQQKPGKAPKLLIYYTSRLHSGVPS |
| | VL-3 | RFSGSGSGTDFTLTISSLQPEDFATYYCQQGNTLPFTFGQGTKLEIK |
| 105 | TLA0015- | DIQMTQSPSSLSASVGDRVTITCRASQDISNYLNWYQQKPGKAPKLLIYYTSRLHSGVPS |
| | VL-4 | RFSGSGSGTDYTLTISSLQPEDFATYFCQQGNTLPFTFGQGTKLEIK |
| 106 | TLA0015- | DIQMTQSPSSLSASVGDRVTITCRASQDISNYLNWYQQKPGKAVKLLIYYTSRLHSGVPS |
| | VL-5 | RFSGSGSGTDYTLTISSLQPEDFATYFCQQGNTLPFTFGQGTKLEIK |
| 107 | TLA-0238- | NY[ILMYW]M[NHS] |
| | HCDR1 | |
| 108 | TLA-0238- | NYWMN |
| | HCDR1-1 | |
| 109 | TLA-0238- | NYWMH |
| | HCDR1-2 | |
| 110 | TLA-0238- | NYWMS |
| | HCDR1-3 | |
| 111 | TLA-0238- | NYIMN |
| | HCDR1-4 | |
| 112 | TLA-0238- | NYLMN |
| | HCDR1-5 | |
| 113 | TLA-0238- | NYMMN |
| | HCDR1-6 | |
| 114 | TLA-0238- | NYYMN |
| | HCDR1-7 | |
| 115 | TLA-0238- | QIRLKSDNYATHYA[ADE][PS]VKG |
| | HCDR2 | |
| 116 | TLA-0238- | QIRLKSDNYATHYAESVKG |
| | HCDR2-1 | |
| 117 | TLA-0238- | QIRLKSDNYATHYADSVKG |
| | HCDR2-2 | |
| 118 | TLA-0238- | QIRLKSDNYATHYAASVKG |
| | HCDR2-3 | |
| 119 | TLA-0238- | QIRLKSDNYATHYAAPVKG |
| | HCDR2-4 | |
| 120 | TLA-0238- | LLLRYRDY |
| | HCDR3-1 | |
| 121 | TLA-0238- | [EQ]V[KQ][LV][EV]ESGGG[LV]V[KQ]PG[GR][AS]L[KR]LSC[AIJASGFTFS |
| | HFR1 | |
| 122 | TLA-0238- | EVKVEESGGGLVQPGGSMKLSCIASGFTFS |
| | HFR1-1 | |
| 123 | TLA-0238- | EVQVVESGGGLVKPGGSLRLSCAASGFTFS |
| | HFR1-2 | |
| 124 | TLA-0238- | EVQVVESGGGLVQPGGSLRLSCIASGFTFS |
| | HFR1-3 | |
| 125 | TLA-0238- | EVQLVESGGGLVQPGGSLRLSCAASGFTFS |
| | HFR 1-4 | |
| 126 | TLA-0238- | QVQLVESGGGVVQPGRSLRLSCAASGFTFS |
| | HFR1-5 | |
| 127 | TLA-0238- | EVKVEESGGGLVQPGGSLRLSCIASGFTFS |
| | HFR1-6 | |
| 128 | TLA-0238- | EVKVEESGGGLVQPGGSLKLSCIASGFTFS |
| | HFR1-7 | |
| 129 | TLA-0238- | EVKVEESGGGLVQPGGALRLSCIASGFTFS |
| | HFR1-8 | |
| 130 | TLA-0238- | EVKLEESGGGLVQPGGSLKLSCIASGFTFS |
| | HFR1-9 | |
| 131 | TLA-0238- | EVQLVESGGGLVKPGGSLRLSCAASGFTFS |
| | HFR1-10 | |
| 132 | TLA-0238- | EVKLEESGGGLVQPGGALRLSCIASGFTFS |
| | HFR1-11 | |
| 133 | TLA-0238- | WVRQ[AS]P[EG]KGLEWV[AG] |
| | HFR2 | |
| 134 | TLA-0238- | WVRQSPEKGLEWVA |
| | HFR2-1 | |
| 135 | TLA-0238- | WVRQAPGKGLEWVA |
| | HFR2-2 | |
| 136 | TLA-0238- | [NR]F[AT]ISRD[DN][AS]K[NS][ST][ALV]YLQMNSL[KR][ADT]ED[MT]AVYYCTP |
| | HFR3 | |
| 137 | TLA-0238- | NFTISRDDSKSSVYLQMNNLRAEDTGIYYCTP |
| | HFR3-1 | |
| 138 | TLA-0238- | NFTISRDDSKSTVYLQMNSLKTEDTAVYYCTP |
| | HFR3-2 | |
| 139 | TLA-0238- | NFTISRDNAKSSVYLQMNSLRAEDTAVYYCTP |
| | HFR3-3 | |
| 140 | TLA-0238- | RFTISRDDSKSTVYLQMNSLKTEDTAVYYCTP |
| | HFR3-4 | |
| 141 | TLA-0238- | NFAISRDDSKSTVYLQMNSLKTEDTAVYYCTP |
| | HFR3-5 | |
| 142 | TLA-0238- | RFTISRDNSKNTLYLQMNSLRAEDTAVYYCTP |
| | HFR3-6 | |
| 143 | TLA-0238- | RFTISRDNSKNTLYLQMNSLKTEDTAVYYCTP |
| | HFR3-7 | |
| 144 | TLA-0238- | RFTISRDDSKNSLYLQMNSLRDEDTAVYYCTP |
| | HFR3-8 | |
| 145 | TLA-0238- | RFTISRDDSKNTLYLQMNSLRAEDTAVYYCTP |
| | HFR3-9 | |
| 146 | TLA-0238- | RFTISRDDSKNSLYLQMNSLRAEDMAVYYCTP |
| | HFR3-10 | |
| 147 | TLA-0238- | RFTISRDDSKNTAYLQMNSLRAEDTAVYYCTP |
| | HFR3-11 | |
| 148 | TLA-0238- | RFTISRDDSKNTLYLQMNSLKTEDTAVYYCTP |
| | HFR3-12 | |
| 149 | TLA-0238- | WGQGT[LMT][LV]TVSS |
| | HFR4 | |
| 150 | TLA-0238- | WGQGTTLTVSS |
| | HFR4-1 | |
| 151 | TLA-0238- | WGQGTLLTVSS |
| | HFR4-3 | |
| 152 | TLA-0238- | WGQGTMVTVSS |
| | HFR4-4 | |
| 153 | TLA-0238- | WGQGTTVTVSS |
| | HFR4-5 | |
| 154 | TLA0238- | SASSSVSY[LM][AHT] |
| | LCDR1 | |
| 155 | TLA0238- | SASSSVSYMH |
| | LCDR1-1 | |
| 156 | TLA0238- | SASSSVSYLH |
| | LCDR1-2 | |
| 157 | TLA0238- | SASSSVSYLA |
| | LCDR1-3 | |
| 158 | TLA0238- | SASSSVSYLT |
| | LCDR1-4 | |
| 159 | TLA0238- | DTSN[LR]A[ST] |
| | LCDR2 | |
| 160 | TLA0238- | DTSNLAS |
| | LCDR2-1 | |
| 161 | TLA0238- | DTSNRAS |
| | LCDR2-2 | |
| 162 | TLA0238- | DTSNLAT |
| | LCDR2-3 | |
| 163 | TLA0238- | FQE[NSE][GEA]HPFT |
| | LCDR3 | |
| 164 | TLA0238- | FQENGHPFT |
| | LCDR3-1 | |
| 165 | TLA0238- | FQESGHPFT |
| | LCDR3-2 | |
| 166 | TLA0238- | FQENEHPFT |
| | LCDR3-3 | |
| 167 | TLA0238- | FQEEGHPFT |
| | LCDR3-4 | |
| 168 | TLA0238- | FQENAHPFT |
| | LCDR3-5 | |
| 169 | TLA0238- | [DE][INTV][QV][LM]TQSP[AS][FIST][LMV]S[ALV][ST][PV]G[DE][KR][AV]T[IM][ST]C |
| | LFR1 | |
| 170 | TLA0238- | ENVLTQSPAIMSASPGEKVTMTC |
| | LFR1-1 | |
| 171 | TLA0238- | ENVLTQSPATLSLSPGERATMSC |
| | LFR1-2 | |
| 172 | TLA0238- | DNQLTQSPSSLSASVGDRVTITC |
| | LFR1-3 | |
| 173 | TLA0238- | DIQMTQSPSSVSASVGDRVTITC |
| | LFR1- | |
| | 4/TLA0241- | |
| | LFR1-2 | |
| 174 | TLA0238- | ENVLTQSPAIMSATPGEKVTITC |
| | LFR1-5 | |
| 175 | TLA0238- | EVVLTQSPAFLSVTPGEKVTITC |
| | LFR1-6 | |
| 176 | TLA0238- | ENVLTQSPAFMSATPGEKVTITC |
| | LFR1-7 | |
| 177 | TLA0238- | EIVLTQSPAFMSATPGEKVTITC |
| | LFR1-8 | |
| 178 | TLA0238- | ETVLTQSPAFMSATPGEKVTITC |
| | LFR1-9 | |
| 179 | TLA0238- | ENVLTQSPAFLSVTPGEKVTITC |
| | LFR1-10 | |
| 180 | TLA0238- | WYQQKP[DG][KQ][AS]P[KQR]LWIY |
| | LFR2 | |
| 181 | TLA0238- | WYQQKSSTSPKLWIY |
| | LFR2-1 | |
| 182 | TLA0238- | WYQQKPGQAPRLWIY |
| | LFR2-2 | |
| 183 | TLA0238- | WYQQKPGKAPKLWIY |
| | LFR2-3 | |
| 184 | TLA0238- | WYQQKPGQSPQLWIY |
| | LFR2-4 | |
| 185 | TLA0238- | WYQQKPGQSPRLWIY |
| | LFR2-5 | |
| 186 | TLA0238- | WYQQKPGKSPKLWIY |
| | LFR2-6 | |
| 187 | TLA0238- | WYQQKPDQSPKLWIY |
| | LFR2-7 | |
| 188 | TLA0238- | WYQQKPGQSPKLWIY |
| | LFR2-8 | |
| 189 | TLA0238- | GVP[ADGS]RFSGSGSG[NT][DS][FY]TLTISS[LM][EQ][AP]ED[AFV]A[TV]YYC |
| | LFR3 | |
| 190 | TLA0238- | GVPGRFSGSGSGNSYSLTISSMEAEDVATYYC |
| | LFR3-1 | |
| 191 | TLA0238- | GVPARFSGSGSGNDYTLTISSMEPEDFAVYYC |
| | LFR3-2 | |
| 192 | TLA0238- | GVPGRFSGSGSGNDYTLTISSLQPEDFATYYC |
| | LFR3-3 | |
| 193 | TLA0238- | GVPDRFSGSGSGTDYTLTISSLQPEDVATYYC |
| | LFR3-5 | |
| 194 | TLA0238- | GVPDRFSGSGSGTDYTLTISSLQPEDFAVYYC |
| | LFR3-6 | |
| 195 | TLA0238- | GVPSRFSGSGSGTSYTLTISSLQPEDVATYYC |
| | LFR3-7 | |
| 196 | TLA0238- | GVPDRFSGSGSGTDYTLTISSLEAEDAATYYC |
| | LFR3-8 | |
| 197 | TLA0238- | GVPSRFSGSGSGTDYTLTISSLQPEDFATYYC |
| | LFR3-9 | |
| 198 | TLA0238 | FG[GQ]GTK[LV]E[IM]K |
| | LFR4 | |
| 199 | TLA0238- | FGGGTKLEMK |
| | LFR4-1 | |
| 200 | TLA0238- | FGGGTKVEMK |
| | LFR4-2 | |
| 201 | TLA0238- | FGQGTKVEIK |
| | LFR4- | |
| | 4/TLA0241- | |
| | LFR4-1 | |
| 202 | TLA0238- | EVKVEESGGGLVQPGGSMKLSCIASGFTFSNYWMNWVRQSPEKGLEWVAQIRLKSDNY |
| | VH-1 | ATHYAESVKGNFTISRDDSKSSVYLQMNNLRAEDTGIYYCTPLLLRYRDYWGQGTTLTV |
| | | SS |
| 203 | TLA0238- | EVQVVESGGGLVKPGGSLRLSCAASGFTFSNYWMNWVRQAPGKGLEWVAQIRLKSDN |
| | VH-2 | YATHYAESVKGNFTISRDDSKSTVYLQMNSLKTEDTAVYYCTPLLLRYRDYWGQGTLVT |
| | | VSS |
| 204 | TLA0238- | EVQVVESGGGLVQPGGSLRLSCIASGFTFSNYWMNWVRQAPGKGLEWVAQIRLKSDNY |
| | VH-3 | ATHYAESVKGNFTISRDNAKSSVYLQMNSLRAEDTAVYYCTPLLLRYRDYWGQGTLLTV |
| | | SS |
| 205 | TLA0238- | EVQVVESGGGLVKPGGSLRLSCAASGFTFSNYWMNWVRQAPGKGLEWVAQIRLKSDN |
| | VH-4 | YATHYAESVKGRFTISRDDSKSTVYLQMNSLKTEDTAVYYCTPLLLRYRDYWGQGTLVT |
| | | VSS |
| 206 | TLA0238- | EVQVVESGGGLVKPGGSLRLSCAASGFTFSNYWMNWVRQAPGKGLEWVAQIRLKSDN |
| | VH-5 | YATHYAESVKGNFAISRDDSKSTVYLQMNSLKTEDTAVYYCTPLLLRYRDYWGQGTLVT |
| | | VSS |
| 207 | TLA0238- | QVQLVESGGGVVQPGRSLRLSCAASGFTFSNYWMNWVRQAPGKGLEWVAQIRLKSDN |
| | VH-6 | YATHYADSVKGRFTISRDNSKNTLYLQMNSLRAEDTAVYYCTPLLLRYRDYWGQGTMV |
| | | TVSS |
| 208 | TLA0238- | EVKVEESGGGLVQPGGSLRLSCIASGFTFSNYWMHWVRQAPGKGLEWVGQIRLKSDNY |
| | VH-7 | ATHYAASVKGRFTISRDNSKNTLYLQMNSLKTEDTAVYYCTPLLLRYRDYWGQGTLVTV |
| | | SS |
| 209 | TLA0238- | EVKVEESGGGLVQPGGSLKLSCIASGFTFSNYWMHWVRQAPGKGLEWVGQIRLKSDNY |
| | VH-8 | ATHYAASVKGRFTISRDDSKNSLYLQMNSLRDEDTAVYYCTPLLLRYRDYWGQGTLVTV |
| | | SS |
| 210 | TLA0238- | EVKVEESGGGLVQPGGALRLSCIASGFTFSNYWMHWVRQAPGKGLEWVGQIRLKSDN |
| | VH-9 | YATHYAASVKGRFTISRDDSKNTLYLQMNSLRAEDTAVYYCTPLLLRYRDYWGQGTLVT |
| | | VSS |
| 211 | TLA0238- | EVKVEESGGGLVQPGGSLRLSCIASGFTFSNYWMHWVRQAPGKGLEWVGQIRLKSDNY |
| | VH-10 | ATHYAASVKGRFTISRDDSKNSLYLQMNSLRAEDMAVYYCTPLLLRYRDYWGQGTLVT |
| | | VSS |
| 212 | TLA0238- | EVQLVESGGGLVQPGGSLRLSCAASGFTFSNYWMNWVRQAPGKGLEWVAQIRLKSDN |
| | VH-11 | YATHYADSVKGRFTISRDNSKNTLYLQMNSLRAEDTAVYYCTPLLLRYRDYWGQGTMV |
| | | TVSS |
| 213 | TLA0238- | EVKLEESGGGLVQPGGSLKLSCIASGFTFSNYWMHWVRQAPGKGLEWVGQIRLKSDNY |
| | VH-12 | ATHYAASVKGRFTISRDDSKNTAYLQMNSLRAEDTAVYYCTPLLLRYRDYWGQGTLVTV |
| | | SS |
| 214 | TLA0238- | EVQLVESGGGLVKPGGSLRLSCAASGFTFSNYWMNWVRQAPGKGLEWVAQIRLKSDN |
| | VH-13 | YATHYAAPVKGRFTISRDDSKNTLYLQMNSLKTEDTAVYYCTPLLLRYRDYWGQGTTVT |
| | | VSS |
| 215 | TLA0238- | EVKLEESGGGLVQPGGALRLSCIASGFTFSNYWMSWVRQAPGKGLEWVGQIRLKSDNY |
| | VH-14 | ATHYAASVKGRFTISRDDSKNTLYLQMNSLRAEDTAVYYCTPLLLRYRDYWGQGTLVTV |
| | | SS |
| 216 | TLA0238- | EVQLVESGGGLVQPGGSLRLSCAASGFTFSNYIMNWVRQAPGKGLEWVAQIRLKSDNY |
| | VH-15 | ATHYADSVKGRFTISRDNSKNTLYLQMNSLRAEDTAVYYCTPLLLRYRDYWGQGTLVTV |
| | | SS |
| 217 | TLA0238- | EVQLVESGGGLVQPGGSLRLSCAASGFTFSNYLMNWVRQAPGKGLEWVAQIRLKSDNY |
| | VH-16 | ATHYADSVKGRFTISRDNSKNTLYLQMNSLRAEDTAVYYCTPLLLRYRDYWGQGTLVTV |
| | | SS |
| 218 | TLA0238- | EVQLVESGGGLVQPGGSLRLSCAASGFTFSNYMMNWVRQAPGKGLEWVAQIRLKSDNY |
| | VH-17 | ATHYADSVKGRFTISRDNSKNTLYLQMNSLRAEDTAVYYCTPLLLRYRDYWGQGTLVTV |
| | | SS |
| 219 | TLA0238- | EVQLVESGGGLVQPGGSLRLSCAASGFTFSNYWMNWVRQAPGKGLEWVAQIRLKSDN |
| | VH-18 | YATHYADSVKGRFTISRDNSKNTLYLQMNSLRAEDTAVYYCTPLLLRYRDYWGQGTLVT |
| | | VSS |
| 220 | TLA0238- | EVQLVESGGGLVQPGGSLRLSCAASGFTFSNYYMNWVRQAPGKGLEWVAQIRLKSDNY |
| | VH-19 | ATHYADSVKGRFTISRDNSKNTLYLQMNSLRAEDTAVYYCTPLLLRYRDYWGQGTLVTV |
| | | SS |
| 221 | TLA0238- | ENVLTQSPAIMSASPGEKVTMTCSASSSVSYMHWYQQKSSTSPKLWIYDTSNLASGVPG |
| | VL-1 | RFSGSGSGNSYSLTISSMEAEDVATYYCFQENGHPFTFGGGTKLEMK |
| 222 | TLA0238- | ENVLTQSPATLSLSPGERATMSCSASSSVSYMHWYQQKPGQAPRLWIYDTSNLASGVPA |
| | VL-2 | RFSGSGSGNDYTLTISSMEPEDFAVYYCFQENGHPFTFGGGTKVEMK |
| 223 | TLA0238- | DNQLTQSPSSLSASVGDRVTITCSASSSVSYMHWYQQKPGKAPKLWIYDTSNLASGVPG |
| | VL-3 | RFSGSGSGNDYTLTISSLQPEDFATYYCFQENGHPFTFGQGTKLEIK |
| 224 | TLA0238- | ENVLTQSPAIMSATPGEKVTITCSASSSVSYLHWYQQKPGQSPQLWIYDTSNRASGVPDR |
| | VL-4 | FSGSGSGTDYTLTISSLQPEDVATYYCFQENGHPFTFGGGTKLEMK |
| 225 | TLA0238- | EVVLTQSPAFLSVTPGEKVTITCSASSSVSYLAWYQQKPGQSPRLWIYDTSNRASGVPDR |
| | VL-5 | FSGSGSGTDYTLTISSLQPEDFAVYYCFQENGHPFTFGGGTKLEMK |
| 226 | TLA0238- | ENVLTQSPAFMSATPGEKVTITCSASSSVSYLAWYQQKPGKSPKLWIYDTSNLATGVPSR |
| | VL-6 | FSGSGSGTSYTLTISSLQPEDVATYYCFQENGHPFTFGGGTKLEMK |
| 227 | TLA0238- | EIVLTQSPAFMSATPGEKVTITCSASSSVSYLHWYQQKPDQSPKLWIYDTSNRASGVPDR |
| | VL-7 | FSGSGSGTDYTLTISSLEAEDAATYYCFQENGHPFTFGGGTKLEMK |
| 228 | TLA0238- | ETVLTQSPAFMSATPGEKVTITCSASSSVSYLTWYQQKPGQSPKLWIYDTSNLASGVPDR |
| | VL-8 | FSGSGSGTDYTLTISSLEAEDAATYYCFQENGHPFTFGGGTKLEMK |
| 229 | TLA0238- | DIQMTQSPSSVSASVGDRVTITCSASSSVSYMHWYQQKPGKAPKLWIYDTSNLASGVPS |
| | VL-9 | RFSGSGSGTDFTLTISSLQPEDFATYYCFQENGHPFTFGQGTKVEIK |
| 230 | TLA0238- | ENVLTQSPAFLSVTPGEKVTITCSASSSVSYLHWYQQKPDQSPKLWIYDTSNLATGVPSRF |
| | VL-10 | SGSGSGTDYTLTISSLQPEDFATYYCFQENGHPFTFGGGTKLEMK |
| 231 | TLA0238- | DIQMTQSPSSVSASVGDRVTITCSASSSVSYMHWYQQKPGKAPKLWIYDTSNLASGVPS |
| | VL-11 | RFSGSGSGTDFTLTISSLQPEDFATYYCFQESGHPFTFGQGTKVEIK |
| 232 | TLA0238- | DIQMTQSPSSVSASVGDRVTITCSASSSVSYMHWYQQKPGKAPKLWIYDTSNLASGVPS |
| | VL-12 | RFSGSGSGTDFTLTISSLQPEDFATYYCFQENEHPFTFGQGTKVEIK |
| 233 | TLA0238- | DIQMTQSPSSVSASVGDRVTITCSASSSVSYMHWYQQKPGKAPKLWIYDTSNLASGVPS |
| | VL-13 | RFSGSGSGTDFTLTISSLQPEDFATYYCFQEEGHPFTFGQGTKVEIK |
| 234 | TLA0238- | DIQMTQSPSSVSASVGDRVTITCSASSSVSYMHWYQQKPGKAPKLWIYDTSNLASGVPS |
| | VL-14 | RFSGSGSGTDFTLTISSLQPEDFATYYCFQENAHPFTFGQGTKVEIK |
| 235 | TLA0241- | DYY[LM][HN] |
| | HCDR1 | |
| 236 | TLA0241- | DYYLN |
| | HCDR1-1 | |
| 237 | TLA0241- | DYYMH |
| | HCDR1-2 | |
| 238 | TLA0241- | DINPNNGRTTY[AN][DQ][KS][FV][KQT]G |
| | HCDR2 | |
| 239 | TLA0241- | DINPNNGRTTYNQKFTG |
| | HCDR2-1 | |
| 240 | TLA0241- | DINPNNGRTTYADSVKG |
| | HCDR2-2 | |
| 241 | TLA0241- | DINPNNGRTTYNQKFQG |
| | HCDR2-3 | |
| 242 | TLA0241- | EGGYGWYFDV |
| | HCDR3-1 | |
| 243 | TLA0241- | [EQ][ILV]QL[LQV][EQ]SG[AGP][EG][ELV][KV][KQ]PG[AGR]S[LV]R[IL]SC[AKS]ASGYT |
| | HFR1 | FT |
| 244 | TLA0241- | EVQLVESGGGLVQPGGSLRLSCSASGYTFT |
| | HFR1-1 | |
| 245 | TLA0241- | QVQLVESGGGVVQPGRSLRLSCAASGYTFT |
| | HFR1-2 | |
| 246 | TLA0241- | EVQLQQSGPEVKKPGASVRISCKASGYTFT |
| | HFR1-3 | |
| 247 | TLA0241- | ELQLQQSGPEVKKPGASVRISCKASGYTFT |
| | HFR1-4 | |
| 248 | TLA0241- | EVQLLESGGGLVQPGGSLRLSCAASGYTFT |
| | HFR1-5 | |
| 249 | TLA0241- | EIQLQQSGAEEKKPGASVRISCKASGYTFT |
| | HFR1-6 | |
| 250 | TLA0241- | ELQLQQSGAEVKKPGASVRISCKASGYTFT |
| | HFR1-7 | |
| 251 | TLA0241- | WV[QR]QAPG[EKQ]GLEWIG |
| | HFR2 | |
| 252 | TLA0241- | WVRQAPGKGLEWIG |
| | HFR2-1 | |
| 253 | TLA0241- | WVRQAPGQGLEWIG |
| | HFR2-2 | |
| 254 | TLA0241- | WVQQAPGKGLEWIG |
| | HFR2-3 | |
| 255 | TLA0241- | WVRQAPGEGLEWIG |
| | HFR2-4 | |
| 256 | TLA0241- | R[FV]TI[ST][AR]D[KNT]S[KST][NS]T[AL]Y[LM][EQ][LM][NRS]SL[RT][AS][DE]D[MT]A |
| | HFR3 | VYYCAR |
| 257 | TLA0241- | RFTISRDNSKNTLYLQMSSLRAEDTAVYYCAR |
| | HFR3-1 | |
| 258 | TLA0241- | RFTISRDNSKNTLYLQMNSLRAEDTAVYYCAR |
| | HFR3-2 | |
| 259 | TLA0241- | RVTITRDTSSSTAYMELRSLRSDDTAVYYCAR |
| | HFR3-3 | |
| 260 | TLA0241- | RVTITADKSTSTAYMELRSLRSDDTAVYYCAR |
| | HFR3-4 | |
| 261 | TLA0241- | RVTITADKSTSTAYMELRSLTSDDTAVYYCAR |
| | HFR3-5 | |
| 262 | TLA0241- | RVTITADKSTSTAYMELRSLRSDDMAVYYCAR |
| | HFR3-6 | |
| 263 | TLA0241- | WG[QRT]GT[LMT]VTVSS |
| | HFR4 | |
| 264 | TLA0241- | WGRGTLVTVSS |
| | HFR4-1 | |
| 265 | TLA0241- | WGQGTMVTVSS |
| | HFR4-2 | |
| 266 | TLA0241- | WGTGTLVTVSS |
| | HFR4-3 | |
| 267 | TLA0241- | WGQGTTVTVSS |
| | HFR4-4 | |
| 268 | TLA0241- | WGTGTTVTVSS |
| | HFR4-5 | |
| 269 | TLA0241- | S[AT]SSSIISNY[LS][AH] |
| | LCDR1 | |
| 270 | TLA0241- | STSSSIISNYSH |
| | LCDR1-1 | |
| 271 | TLA0241- | SASSSIISNYLH |
| | LCDR1-2 | |
| 272 | TLA0241- | SASSSIISNYSA |
| | LCDR1-3 | |
| 273 | TLA0241- | SASSSIISNYLA |
| | LCDR1-4 | |
| 274 | TLA0241- | KTSN[LR]PS |
| | LCDR2 | |
| 275 | TLA0241- | KTSNLPS |
| | LCDR2-1 | |
| 276 | TLA0241- | KTSNRPS |
| | LCDR2-2 | |
| 277 | TLA0241- | QQGSDMPIT |
| | LCDR3-1 | |
| 278 | TLA0241- | [ADE]I[QV][LM]TQSP[AGPST][ST][LV]S[AL]S[PV]G[DE][KR]VTITC |
| | LFR1 | |
| 279 | TLA0241- | EIVLTQSPTTLSLSPGEKVTITC |
| | LFR1-3 | |
| 280 | TLA0241- | EIVLTQSPPTLSLSPGEKVTITC |
| | LFR1-4 | |
| 281 | TLA0241 | EIVLTQSPGTLSLSPGEKVTITC |
| | LFR1-5 | |
| 282 | TLA0241- | AIQLTQSPSSLSASVGDRVTITC |
| | LFR1-6 | |
| 283 | TLA0241- | EIVLTQSPATLSLSPGEKVTITC |
| | LFR1-7 | |
| 284 | TLA0241- | WYQQKP[DG][KQ][AS]P[KQ]LLIY |
| | LFR2 | |
| 285 | TLA0241- | WYQQKPGKSPKLLIY |
| | LFR2-2 | |
| 286 | TLA0241- | WYQQKPGQSPQLLIY |
| | LFR2-3 | |
| 287 | TLA0241- | WYQQKPDQSPKLLIY |
| | LFR2-4 | |
| 288 | TLA0241- | GVP[ADS]RFSGSGSGT[DES]FTLTI[NS]SL[EQ][AP]ED[AF]ATYYC |
| | LFR3 | |
| 289 | TLA0241- | GVPSRFSGSGSGTEFTLTISSLQPEDFATYYC |
| | LFR3-1 | |
| 290 | TLA0241- | GVPSRFSGSGSGTDFTLTINSLEAEDAATYYC |
| | LFR3-3 | |
| 291 | TLA0241- | GVPDRFSGSGSGTDFTLTINSLEAEDAATYYC |
| | LFR3-4 | |
| 292 | TLA0241- | GVPARFSGSGSGTDFTLTINSLEAEDAATYYC |
| | LFR3-5 | |
| 293 | TLA0241- | GVPSRFSGSGSGTSFTLTINSLEAEDAATYYC |
| | LFR3-6 | |
| 294 | TLA0241- | FG[AQ]GT[KR][LV]E[IL]K |
| | LFR4 | |
| 295 | TLA0241- | FGAGTRLELK |
| | LFR4-2 | |
| 296 | TLA0241- | FGAGTKLELK |
| | LFR4-3 | |
| 297 | TLA0241- | EFQLQQSGPELVKPGASVRISCKASGYTFTDYYLNWVKQSRGESLEWIGDINPNNGRTT |
| | VH-1 | YNQKFTGKATWTVDKSSSTVYMELRSLTSDDSAVYYCAREGGYGWYFDVWGTGTTVT |
| | | VSS |
| 298 | TLA0241- | EVQLVESGGGLVQPGGSLRLSCSASGYTFTDYYLNWVRQAPGKGLEWIGDINPNNGRTT |
| | VH-2 | YADSVKGRFTISRDNSKNTLYLQMSSLRAEDTAVYYCAREGGYGWYFDVWGRGTLVTV |
| | | SS |
| 299 | TLA0241- | QVQLVESGGGVVQPGRSLRLSCAASGYTFTDYYLNWVRQAPGKGLEWIGDINPNNGRT |
| | VH-3 | TYADSVKGRFTISRDNSKNTLYLQMNSLRAEDTAVYYCAREGGYGWYFDVWGQGTMV |
| | | TVSS |
| 300 | TLA0241- | EVQLQQSGPEVKKPGASVRISCKASGYTFTDYYMHWVRQAPGQGLEWIGDINPNNGRT |
| | VH-4 | TYNQKFQGRVTITRDTSSSTAYMELRSLRSDDTAVYYCAREGGYGWYFDVWGTGTTVT |
| | | VSS |
| 301 | TLA0241- | EVQLQQSGPEVKKPGASVRISCKASGYTFTDYYMHWVQQAPGKGLEWIGDINPNNGRT |
| | VH-5 | TYNQKFQGRVTITADKSTSTAYMELRSLRSDDTAVYYCAREGGYGWYFDVWGTGTLVT |
| | | VSS |
| 302 | TLA0241- | ELQLQQSGPEVKKPGASVRISCKASGYTFTDYYMHWVQQAPGKGLEWIGDINPNNGRT |
| | VH-6 | TYNQKFQGRVTITADKSTSTAYMELRSLTSDDTAVYYCAREGGYGWYFDVWGTGTTVT |
| | | VSS |
| 303 | TLA0241- | EVQLLESGGGLVQPGGSLRLSCAASGYTFTDYYLNWVRQAPGKGLEWIGDINPNNGRT |
| | VH-7 | TYADSVKGRFTISRDNSKNTLYLQMNSLRAEDTAVYYCAREGGYGWYFDVWGQGTMV |
| | | TVSS |
| 304 | TLA0241- | EVQLQQSGPEVKKPGASVRISCKASGYTFTDYYMHWVRQAPGEGLEWIGDINPNNGRT |
| | VH-8 | TYNQKFQGRVTITADKSTSTAYMELRSLRSDDTAVYYCAREGGYGWYFDVWGTGTTVT |
| | | VSS |
| 305 | TLA0241- | QVQLVESGGGVVQPGRSLRLSCAASGYTFTDYYLNWVRQAPGKGLEWIGDINPNNGRT |
| | VH-9 | TYADSVKGRFTISRDNSKNTLYLQMNSLRAEDTAVYYCAREGGYGWYFDVWGQGTTV |
| | | TVSS |
| 306 | TLA0241- | EIQLQQSGAEEKKPGASVRISCKASGYTFTDYYMHWVQQAPGKGLEWIGDINPNNGRTT |
| | VH-10 | YNQKFQGRVTITADKSTSTAYMELRSLRSDDTAVYYCAREGGYGWYFDVWGTGTTVTV |
| | | SS |
| 307 | TLA0241- | ELQLQQSGAEVKKPGASVRISCKASGYTFTDYYMHWVRQAPGQGLEWIGDINPNNGRT |
| | VH-11 | TYNQKFQGRVTITADKSTSTAYMELRSLRSDDMAVYYCAREGGYGWYFDVWGTGTLVT |
| | | VSS |
| 308 | TLA0241- | EIVLTQSPTTMAASPGEKITITCSTSSSIISNYSHWYQQKPGFSPKLLIYKTSNLPSGVPARF |
| | VL-1 | SGSGSGTSYSLTIGTMEAEDAATYYCQQGSDMPITFGAGTKLELK |
| 309 | TLA0241- | DIQMTQSPSSLSASVGDRVTITCSTSSSIISNYSHWYQQKPGKAPKLLIYKTSNLPSGVPSR |
| | VL-2 | FSGSGSGTEFTLTISSLQPEDFATYYCQQGSDMPITFGQGTKVEIK |
| 310 | TLA0241- | EIVLTQSPTTLSLSPGEKVTITCSASSSIISNYLHWYQQKPGKSPKLLIYKTSNLPSGVPSRF |
| | VL-3 | SGSGSGTDFTLTINSLEAEDAATYYCQQGSDMPITFGAGTRLELK |
| 311 | TLA0241- | EIVLTQSPPTLSLSPGEKVTITCSASSSIISNYLHWYQQKPGQSPQLLIYKTSNRPSGVPDRF |
| | VL-4 | SGSGSGTDFTLTINSLEAEDAATYYCQQGSDMPITFGAGTRLELK |
| 312 | TLA0241- | EIVLTQSPGTLSLSPGEKVTITCSASSSIISNYLHWYQQKPDQSPKLLIYKTSNLPSGVPSRF |
| | VL-5 | SGSGSGTDFTLTINSLEAEDAATYYCQQGSDMPITFGAGTKLELK |
| 313 | TLA0241- | AIQLTQSPSSLSASVGDRVTITCSTSSSIISNYSHWYQQKPGKAPKLLIYKTSNLPSGVPSRF |
| | VL-6 | SGSGSGTDFTLTISSLQPEDFATYYCQQGSDMPITFGQGTKVEIK |
| 314 | TLA0241- | EIVLTQSPATLSLSPGEKVTITCSASSSIISNYSAWYQQKPGKSPKLLIYKTSNLPSGVPARF |
| | VL-7 | SGSGSGTDFTLTINSLEAEDAATYYCQQGSDMPITFGAGTRLELK |
| 315 | TLA0241- | DIQMTQSPSSVSASVGDRVTITCSTSSSIISNYSHWYQQKPGKAPKLLIYKTSNLPSGVPSR |
| | VL-8 | FSGSGSGTDFTLTISSLQPEDFATYYCQQGSDMPITFGQGTKVEIK |
| 316 | TLA0241- | EIVLTQSPTTLSLSPGEKVTITCSASSSIISNYLHWYQQKPDQSPKLLIYKTSNLPSGVPSRF |
| | VL-9 | SGSGSGTDFTLTINSLEAEDAATYYCQQGSDMPITFGAGTKLELK |
| 317 | TLA0241- | EIVLTQSPPTLSLSPGEKVTITCSASSSIISNYLAWYQQKPGKSPKLLIYKTSNLPSGVPSRF |
| | VL-10 | SGSGSGTSFTLTINSLEAEDAATYYCQQGSDMPITFGAGTKLELK |
| 318 | CK | RTVAAPSVFIFPPSDEQLKSGTASVVCLLNNFYPREAKVQWKVDNALQSGNSQESVTEQ |
| | | DSKDSTYSLSSTLTLSKADYEKHKVYACEVTHQGLSSPVTKSFNRGEC |
| 319 | CH1 | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSC |
| 320 | Fc(L234A, | DKTHTCPPCPAPEAAGGPSVFLFPPKPKDTLMISRTPEVTCVVVDVSHEDPEVKFNWYV |
| | L235A) | DGVEVHNAKTKPREEQYNSTYRVVSVLTVLHQDWLNGKEYKCKVSNKALPAPIEKTIS |
| | | KAKGQPREPQVYTLPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| 321 | Fc(L234A, | DKTHTCPPCPAPEAAGGPSVFLFPPKPKDTLYITREPEVTCVVVDVSHEDPEVKFNWYVD |
| | L235A, | GVEVHNAKTKPREEQYNSTYRVVSVLTVLHQDWLNGKEYKCKVSNKALPAPIEKTISK |
| | M252Y, | AKGQPREPQVYTLPPSRDELTKNQVSLTCLVKGFYPSDIAVEWESNGQPENNYKTTPPVL |
| | S254T, | DSDGSFFLYSKLTVDKSRWQQGNVFSCSVMHEALHNHYTQKSLSLSPGK |
| | T256E) | |
| 322 | RVT-3101- | QVQLVQSGAEVKKPGASVKVSCKASGYDFTYYGISWVRQAPGQGLEWMGWISTYNGN |
| | VH | THYARMLQGRVTMTTDTSTRTAYMELRSLRSDDTAVYYCARENYYGSGAYRGGMDVW |
| | | GQGTTVTVSS |
| 323 | RVT-3101- | EIVLTQSPATLSLSPGERATLSCRASQSVSSYLAWYQQKPGQAPRLLIYDASNRATGIPARF |
| | VL | SGSGSGTDFTLTISSLEPEDFAVYYCQQRSNWPWTFGQGTKVEIK |
| 324 | MK-7240- | QVQLVQSGAEVKKPGASVKVSCKASGFDIQDTYMHWVKQRPGQGLEWMGRIDPASGH |
| | VH | TKYDPKFQVRVTITRDTSTSTVYLELSSLRSEDTAVYYCARSGGLPDVWGQGTTVTVSS |
| 325 | MK-7240- | EIVLTQSPGTLSLSPGERATLSCRASSSVSYMYWYQQKPGQAPRPLIYATSNLASGIPDRES |
| | VL | GSGSGTDFTLTISRLEPEDFAVYYCQQWEGNPRTFGGGTKLEIK |
| 326 | Human | MAEDLGLSFGETASVEMLPEHGSCRPKARSSSARWALTCCLVLLPFLAGLTTYLLVSQLR |
| | TL1A (M1- | AQGEACVQFQALKGQEFAPSHQQVYAPLRADGDKPRAHLTVVRQTPTQHFKNQFPALH |
| | L251) | WEHELGLAFTKNRMNYTNKFLLIPESGDYFIYSQVTFRGMTSECSEIRQAGRPNKPDSIT |
| | | VVITKVTDSYPEPTQLLMGTKSVCEVGSNWFQPIYLGAMFSLQEGDKLMVNVSDISLVD |
| | | YTKEDKTFFGAFLL |
| 327 | Human | LKGQEFAPSHQQVYAPLRADGDKPRAHLTVVRQTPTQHFKNQFPALHWEHELGLAFTK |
| | STL1A (L72- | NRMNYTNKFLLIPESGDYFIYSQVTFRGMTSECSEIRQAGRPNKPDSITVVITKVTDSYPE |
| | L251) | PTQLLMGTKSVCEVGSNWFQPIYLGAMFSLQEGDKLMVNVSDISLVDYTKEDKTFFGAF |
| | | LL |
| 328 | Cyno sTL1A | LKGQEFAPSHQQVYAPLRADGDKPRAHLTVVRQTPTQHLKNQFPALHWEHELGLAFTK |
| | (L72-L251) | NRMNYTNKFLLIPESGDYFVYSQVTFRGMTSECSEIRQAGRPNKPDSITVVITKVTDSYP |
| | | EPTQLLMGTKSVCEVGSNWFQPIYLGAMFSLQEGDKLMVNVSDISLVDYTK |
| | | EDKTFFGAFLL |
| 329 | Mouse | AGQLRVPGKDCMLRAITEERSEPSPQQVYSPPRGKPRAHLTIKKQTPAPHLKNQLSALH |
| | STL1A (A61- | WEHDLGMAFTKNGMKYINKSLVIPESGDYFIYSQITFRGTTSVCGDISRGRRPNKPDSITV |
| | L252) | VITKVADSYPEPARLLTGSKSVCEISNNWFQSLYLGAMFSLEEGDRLMVNVSDISLVDYT |
| | | KEDKTFFGAFLL |
| 330 | Rat | TGQLRIPGKDCMFPTVTEERSAPSAQPVYTPSRDKPKAHLTIMRQTPVPHLKNELAALH |
| | STL1A(T61- | WENNLGMAFTKNRMNYTNKFLVIPESGDYFIYSQITFRGTTSECGDISRVRRPKKPDSITV |
| | I252) | VITKVADSYPEPAHLLTGTKSVCEISSNWFQPIYLGAMESLEEGDRLMVNVSDISLVDYTK |
| | | EDKTFFGAFLI |
| 331 | Human DR3- | QGGTRSPRCDCAGDFHKKIGLFCCRGCPAGHYLKAPCTEPCGNSTCLVCPQDTFLAWEN |
| | ECD(Q25- | HHNSECARCQACDEQASQVALENCSAVADTRCGCKPGWFVECQVSQCVSSSPFYCQPC |
| | Q199) | LDCGALHRHTRLLCSRRDTDCGTCLPGFYEHGDGCVSCPTSTLGSCPERCAAVCGWRQ |
| 332 | TLA0238- | EVQLVESGGGLVKPGGSLRLSCAASGFTFSNYWMNWVRQAPGKGLEWVGQIRLKSDN |
| | VH20 | YATHYAESVKGRFTISRDDSKNTLYLQMNSLKTEDTAVYYCTTLLLRYRDYWGQGTLVT |
| | | VSS |
| 333 | TLA0238- | EVQLVESGGGLVKPGGSLRLSCAASGFTFSNYWMNWVRQAPGKGLEWVGQIRLKSDN |
| | VH21 | YATHYAESVKGRFTISRDDSKNTLYLQMNSLKTEDTAVYYCTPLLLRYRDYWGQGTLVT |
| | | VSS |
| 334 | TLA0238- | EVQLVESGGGLVKPGGSLRLSCAASGFTFSNYWMNWVRQAPGKGLEWVAQIRLKSDN |
| | VH22 | YATHYAESVKGRFTISRDDSKNTLYLQMNSLKTEDTAVYYCTPLLLRYRDYWGQGTLVT |
| | | VSS |
| 335 | TLA0238- | EIVLTQSPATLSLSPGERATLSCSASSSVSYMHWYQQKPGQSPRLWIYDTSNLASGIPARFS |
| | VL-15 | GSGSGTDYTLTISSLEPEDFAVYYCFQENAHPFTFGGGTKVEIK |
| 336 | TLA0238- | EIVLTQSPATLSLSPGERATLSCSASSSVSYMHWYQQKPGQSPRLWIYDTSNLASGVPARF |
| | VL-16 | SGSGSGTDYTLTISSLEPEDFAVYYCFQENAHPFTFGGGTKVEIK |
| 337 | TLA0238 | DIQMTQSPSSLSASVGDRVTITCSASSSVSYMHWYQQKPGKAPKLWIYDTSNLASGVPS |
| | VL-17 | RFSGSGSGTDYTLTISSLQPEDFATYYCFQENAHPFTFGQGTKLEIK |
| 338 | TLA0238- | DIQLTQSPSSLSASVGDRVTITCSASSSVSYMHWYQQKPGKAPKLWIYDTSNLASGVPSR |
| | VL-18 | FSGSGSGTDYTLTISSLQPEDFATYYCFQENAHPFTFGQGTKLEIK |
| 339 | HC | EIQLVESGGGLIQPGGSLRISCAVSGFSLSSYGVDWVRQAPGKGLEWLGVIWGFGGTNY |
| | | NSALKSRLTISKDNSKNTVYLQMNSLRAEDTAVYYCASGNFDAMDYWGQGTLVTVSSA |
| | | STKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGGPS |
| | | VFLFPPKPKDTLYITREPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 340 | LC | DIQMTQSPSSLSASVGDRVTITCRASQDISNYLNWYQQKPGKAVKLLIYYTSRLHSGVPS |
| | | RFSGSGSGTDYTLTISSLQPEDFATYFCQQGNTLPFTFGQGTKLEIKRTVAAPSVFIFPPSDE |
| | | QLKSGTASVVCLLNNFYPREAKVQWKVDNALQSGNSQESVTEQDSKDSTYSLSSTLTLS |
| | | KADYEKHKVYACEVTHQGLSSPVTKSFNRGEC |
| 341 | Fc(No | DKTHTCPPCPAPELLGGPSVFLFPPKPKDTLMISRTPEVTCVVVDVSHEDPEVKFNWYVD |
| | mutation) | GVEVHNAKTKPREEQYNSTYRVVSVLTVLHQDWLNGKEYKCKVSNKALPAPIEKTISK |
| | | AKGQPREPQVYTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| 342 | mice-1 | QIQLVQSGPELKKPGETVKISCKASGYTFTTYGMNWVKQSPGKGLKWMGWINTYSGVP |
| | | TYADDFKGRFAFSLETSVNTAYLQINSLKTDDTATYFCAKDAYDNYAMDYWGQGTSV |
| | | TVSS |
| 343 | mice-2 | QIQLKESGPGLVAPSQSLSITCTVSGFSLSSYGVDWVRQSPGKGLEWLGVIWGFGGTNY |
| | | NSALKSRLSISKDNSKSQVFLKMNSLQSDDSAIYYCASGNFDAMDYWGQGTSVTVSS |
| 344 | mice-3 | DVQLQESGPGLVKPSQSLSLTCSVTDYSITSGYYWNWIRQFPGNKLEWMGYIHFDGSND |
| | | YNPSLKNRISITRDTSRNQFFLKLNSVTSEDTATYYCAREGTSYFDYWGQGTTLTVSS |
| 345 | mice-4 | QIQLVQSGPELKKPGETVKISCKASGYTFTTYGMSWVKQAPGKYLKWMGWIDTYSGVP |
| | | TYADDFKGRFAFSLETSANTAYLQINNLKNEDTATYFCANSFQFAYWGQGTLVTVSA |
| 346 | mice-5 | DVQLQESGPGLVKPSQSLSLTCSVTGYSITSGYYWNWIRQFPGNKLEWMGCISYDGSNY |
| | | YNPSLKNRISFTRDTSKNQFFLKLNSVTTDDTATYYCVREGTSYVDYWGQGTTLTVSS |
| 347 | mice-6 | DVQLQESGPGLVKPSQSLSLTCSVTDYSITSGYYWNWIRQFPGNKLEWMGYIHYDGSN |
| | | DYNPSLKNRISITRDTSRNQFFLKLNSVTSEDTATYYCAREGTSYFDYWGQGTTLTVSS |
| 348 | mice-7 | QVQLQQSGPELVRPGASVKISCKAPGYTFTSHWMQWVRQRPGQGLEWIGEIFPGSGTTY |
| | | YNEKFQGKATLTVDTSSSTAYMQLSSLTSEDSSVHFCARGEGIYYPARSYGPLGYFDVW |
| | | GTGTTVTVSS |
| 349 | mice-8 | QVQLQQPGAELVMPGASVKLSCKASGYTFTNYWMNWVKQRPRQGLEWIGETDPSNTY |
| | | INYNQKFKGKATLTVDKSSNTAYMQLNSLTSEDSAVYYCARGGLPYYGMDYWGQGTS |
| | | VTVSS |
| 350 | mice-9 | QIQLVQSGPELKKPGETVKISCKASGSAFTTYGMSWVKQAPGEGLKWMGYIITYSGVPT |
| | | YADDFKGRFAFSLETSASTAYLQINNLKNEDTATYFCTNFYYFDYWGQGTTLTVSS |
| 351 | mice-10 | QIQLVQSGPELKKPGETVKISCKASGYTFTTFGMNWVKQAPGKDLKWMGWINTNSGVP |
| | | TYADDFRGRFAFSMETSATTAYLQINNLKNEDTATYFCARGNWEGPGIWFAYWGQGTL |
| | | VTVSA |
| 352 | mice-11 | QVQLQQPGTELVKPGASVKLSCKASGYTFTSYWMHWVKQRPGQGLEWIGNIDPNNDVI |
| | | NYNDKFKSKATLTVDKSSSTAYMQLSSLTSEDSGVYYCARGIYAGYWGQGTLVTVSA |
| 353 | mice-12 | EVQLQQSGAELMKPGASVKLSCKATGYTFTGYWLEWVKQRPGHGLEWIGETLPGSGY |
| | | NKYNEKFKGKATFTADTSSNTAHMQLSSLTTEDSAIYYCARRGSNYPYWYFDVWGTGT |
| | | TVTVSS |
| 354 | mice-13 | QVQLKESGPGLVQPSQSLSITCTVSGFSLTSYGVHWVRQSPGKGLEWLGVIWSGGSTDY |
| | | NAAFISRLSISKDNSKSQVFFKMNSLQADDTAIYYCARNDYDYAMDYWGQGTSVTVSS |
| 355 | mice-14 | QVQLQQSGPELVKPGASVKLSCKASGYTFTSIDINWVKQRPGQGLEWIGWIYPRDGRTD |
| | | YNEKFKGKATLTVDTSSSTAYMELHSLTSEDSAVYFCARYTSSLDYWGQGTTLTVSS |
| 356 | mice-15 | EVKLEESGGGLVQPGGSMKLSCVASGFTFSNYWMNWVRQSPEKGLEWVAQIRLKSDN |
| | | YATHYAESVKGRFTISRDDSKSNVYLQMNNLRAEDTGIYYCTPLLLRSIDYWGQGTTLT |
| | | VSS |
| 357 | mice-16 | EVQLQQSGPELVKPGASVKLSCKASGYTFTSIDINWVKQRPGQGLEWIGWIYPRDGRTD |
| | | YNEKFKGKATLTVDTSSSTAYMELHSLTSEDSAVYFCARYGSSLDYWGQGTTLTVSS |
| 358 | mice-17 | QVQLQQSGPELVKPGASVKLSCKASGYTFTNQDMNWVKQRPGQGLEWIGWIFPRDGR |
| | | THHNENFKGKATLTVDTSSSTAYMELHSLTAEDSAVYFCAIYGFAMDQWGQGTSVTVS |
| | | S |
| 359 | mice-18 | QVQLQQPGPELVKPGASVKLSCKASGYTFTNQDINWVKQRPGQGLEWIGWIFPRDGRT |
| | | HYNENFKGKATLTVDTSSSTAYMELHSLTAEDSAVYFCAIYGIALDHWGQGTSVTVSS |
| 360 | mice-19 | QVQLQQPGPELVKPGASVKLSCKASGYTFTNQDINWVKQRPGQGLEWIGWIFPRDGRT |
| | | HYNENFKGKATLTVDTSSSTAYMELHSLTAEDSAVYFCAIYGAAMDHWGQGTSVTVSS |
| 361 | mice-20 | EVKLEESGGGLVQPGGSMKLSCVASGFTFSNYWMNWVRQSPEKGLEWVAQIRLKSDN |
| | | YATHYAESVKGRFTISRDDSKSSVYLQMNNLRAEDTGIYYCTPLLLRYFDVWGTGTTVT |
| | | VSS |
| 362 | mice-21 | EVQLQQSVAELVRPGASVKLSCTASGFNIEDTYIHWVKQRPEQGLEWIGRIDPAHDKTK |
| | | YAPKFQGKATITADTSSNTAYLQLSSLTSEDTAIYYCASTGPDWWGQGTTLTVSS |
| 363 | mice-22 | QVQLQQSGAELVRPGTSVKMSCKASGYTFTNYWIGWTKQRPGHGLEWIGDIYPGGGYI |
| | | NYNEKFKGKATLTADKSSSTAYMQFSSLTSEDSAIYYCAREGGYGSGQAWFPYWGQGT |
| | | LVTVSA |
| 364 | mice-23 | QVQLQQPGTELVRPGTSVKLSCKASGYTFTSYWMHWVKQRPGQGLEWIGVIEPSDSYT |
| | | DYNQKFKGKATLTVDTSSSTAYMQLSSLTSEDSAVYYCARGDYDEVFAYWGQGTLVT |
| | | VSA |
| 365 | mice-24 | QVQLQQPGAELVRPGTSVKLSCKASGYTFTSYWMHWVKQRPGQGLEWIGVIEPSDSYT |
| | | DYDQKFKGKATLTVDTSSNTAYMQLSSLTSEDSAVYYCSRGDYDEVFAYWGQGTLVT |
| | | VST |
| 366 | mice-25 | QVQLQQSGADLVRPGASVTLSCKASGYTFSDSEIHWVRQTPVHGLEWIGAIDPETGDIA |
| | | YNQMFKDKATLTADKSSSTAYVELRSLTSEDSAVYYCTRYGGNSWHFDVWGTGTTVT |
| | | VSS |
| 367 | mice-26 | EFQLQQSGPELLKPGASVKISCKASGYSFTDYNINWVKQRNGKSLEWIGMINPKYGITD |
| | | YNQKFKGKATLTVDQFSSTAYMQVNSLTSDDSAVYYCAREELGHWCFDVWGTGTTVT |
| | | VSS |
| 368 | mice-27 | QVQLQQSGAELVRPGTSVKVSCKASGYAFTNYLIEWVNQRPGQGLEWIGVINPGSGGT |
| | | KYNEKFKGKATLTADKSSSTAYMQLSSLTSEDSAVYFCAREGSAGPFDYWGQGTTLTV |
| | | SS |
| 369 | mice-28 | EFQLQQSGPELVKPGASVKISCKASGYSFTDYNINWVKQRNGKSLEWIGMINPKYGITD |
| | | YNQKFKGKATLTVDQFSSTAYMQVNSLTSDDSAVYYCAREELGHWCFDVWGTGTTVT |
| | | VSS |
| 370 | mice-29 | EVQLQQSGPVLVKPGASVKMSCEASGYTLTDYYMNWVKQSHGKSLEWIGIINPYNGAS |
| | | NFNQRFRGKASLTFGKSSNTAYMELNSLTSEDSAVYYCASDWDVGYWGQGTALTVSS |
| 371 | mice-30 | EVQLVESGGDLVKPGGSLKLSCAASGFTFSRYGMSWVRQTPDKRLEWVATISSGGSDT |
| | | YYPDSVKGRFTISRDNAKNTLYLQMSSLKSEDTAMYYCARHGYDYGHFDYWGQGTTL |
| | | TVSS |
| 372 | mice-31 | EFQLQQSGPELVKPGASVKISCKASGYSFTDYNINWVKQSNGKSLEWIGVINPNYGITDY |
| | | NQKFKGKATLTVDQSSSTAYMQLNSLTSEDSAVYYCAREELGHWCFDVWGTGTTVTV |
| | | SS |
| 373 | mice-32 | QVTLKESGPGILQPSQTLNLTCSFSGFSLSTFGMGVGWIRQPSGEGLEWLAHIWWDDHE |
| | | YYNPALKSRLTISKDTSKNQVFLKIANVDTADTATYYCNRGFVYWGQGTLVTVSA |
| 374 | mice-33 | QVQLQQSGPELVKPGTSVKLSCKASGYTFTSYDINWMKQRPGQGLEWIGWIFPRDGITK |
| | | YNEKFKDKATLTVDTSSSAAYMELHSLTSEDSAVYFCARYGPAMDYWGQGTSVTVSS |
| 375 | mice-34 | QIQLVQSGPELVKPGASVKVSCKASGYTFTRQDINWVKQRPGQGLEWIGWIYPRDGRTE |
| | | YNEKFKGKVTLTVDTSSSTAYMELHSLTSEDSAVYFCATYGSSLDNWGQGTTLTVSS |
| 376 | mice-35 | EVQLQQSGPELVKPGASVKVSCKASGYTFTRQDINWVKQRPGQGLEWIGWIYPRDGST |
| | | KYNEKFKGKVTLTVDTSSSTAYMELHSLTSEDSAVYFCATYGSSLDNWGQGTTLTVSS |
| 377 | mice-36 | EVQLQQSGPELVKPGASVKLSCKASGYTFTNQDINWVKQRPGQGLEWIGWIFPRDGRT |
| | | HYNENFKGKATLTVDTSSSTAYMELHSLTAEDSAVYFCAIYGIAMDHWGQGTSVTVSS |
| 378 | mice-37 | QIQLVQSGPELVKPGASVKLSCKASGYTFTNYDINWVKQRPGQGLEWIGWIYPRDGSTY |
| | | YNEKFKGKATLTVDTSSTTAYMEFHSLTSEDSAVYFCARYAYALNSWGQGTSVTVSS |
| 379 | mice-38 | QVQLKQSGPELVRPGASVKLSCKASGYSFTSGDINWVKQRPGQGLEWIAWIYPRDGSTE |
| | | YNEKFKGKATLTVDTSSSTAYMELHSLTSEDSAVYFCARYDCAFVYWGQGTLVTVSA |
| 380 | mice-39 | QVQLQQSGPELVKPGASVKLSCKASGYTFTNQDINWVKQRPGQGLEWIGWIFPRDGRT |
| | | HYNENFKGKATLTVDTSSSTAFMELHSLTAEDSAVYFCAIYGIALDHWGQGTSVTVSS |
| 381 | mice-40 | QVQLQQSGPELVKPGASVKLSCKASGYTFTDYDVNWVKQRPGQGLEWIGWNYPRDGR |
| | | TNYNEKFKGKATLTVDISSSTAYMELHSLTSEDSAVYFCARYGSALNVWGTGTSVTVSS |
| 382 | mice-41 | QIQLVQSGPELVKPGASVKLSCKASGYTFTKYDINWVKQRPGQGLEWIGWIYPRDGSTK |
| | | YNEKFKGKATLTVDTSSNTAYMELHSLTSEDSAVYFCARYSAAMDYWGQGTSVTVSS |
| 383 | mice-42 | QVQLQQPGAELGKPGASVKLSCKASGYTFTNYDINWVKQRPGQGLEWIGWNYPRDGR |
| | | TKYNEKFKGKATLTVDTSSSTAYMELHSLTSEDSAVYFCARYGYAMDYWGQGTSVTV |
| | | SS |
| 384 | mice-43 | QVQLQQPGAELGKPGASVKLSCKASGYTFTNYDINWMKLRPGQGLEWIGWNYPRDGR |
| | | TKYNEKFKGKATFTVDTSSSTAYMELHSLTSEDSAVYFCARYGYAMDYWGQGTSVTV |
| | | SS |
| 385 | mice-44 | QIQLVQSGPELVKPGASVKLSCRASGYTFTSYDINWVKQRPGQGLEWIGWIFPRDGRTE |
| | | YNAKFKGKATLTVDTSSNTAYMELHSLTSDDSAVYFCARYGSTLDVLGAGTTVTVSS |
| 386 | mice-45 | EVQLQQSGPELVKPGASVKLSCKASGYTFTNQDINWVKQRPGQGLEWIGWIFPRDGRT |
| | | HYNENFKDKATLTVDTSSSTAYMEVHSLTAEDSAVYFCAIYGIALDHWGQGTSVTVSS |
| 387 | mice-46 | QIQLVQSGPELVKPGASVKLSCKASGYTFTRQDINWVKQRPGQGLEWIGWIYPRDGSTK |
| | | YNEKFKGKVTLTVDTSSSTAYMELHSLTSEDSAVYFCATYGSSLDNWGQGTTLTVSS |
| 388 | mice-47 | QVQLQQSGPELVKPGASVKIPCKTSGYTFTDYNINWVKRSHGKSLEWIGDINPDSGGTS |
| | | YNQRFKDKATLTVDKSSSTAYMELRSLTSGDTAVYYCARWGYYGGRGFDYWGQGTTL |
| | | TVSS |
| 389 | mice-48 | QVQLQQSGAELGKPGASVKLSCKASGYTFTNYDINWMKQRPGQGLEWIGWNYPRDGR |
| | | TKYNEKFKGKATLTVDTSSSTAYMELHSLTSEDSAVYFCARYGYAMDYWGQGTSVTV |
| | | SS |
| 390 | mice-49 | EVQLQQSGPDLVKPGASVKLSCKASGYTFASYDINWVKQRPGQGLEWIGWIYPRDGRT |
| | | DYNEKFKGKATLTVDTSSSTAYMELHSLTSEDSAVYFCARYGYALGYWGQGTSVTVSS |
| 391 | mice-50 | QVQLQQSGPELAKPGASVKLSCKASGYTFTSRDINWVKQRPGQGLEWIGWIYPRDGRT |
| | | NYNEKFKDKATLTVDTSSSTAYMDLHSLTSEDSAVYFCATYGSSLDHWGQGTTLTVSS |
| 392 | mice-51 | EVQLQQSGPELVKPGASVKLSCKASGYTFTNQDINWVKQRPGQGLEWIGWIFPRDGRT |
| | | HYNENFKGKATLTVDTSSSTAYMELHSLTAEDSAVYFCAIYGAAMDHWGQGTSVTVSS |
| 393 | mice-52 | QVQLQQSGPELVKPGTSVKLSCKASGYTFTDQDINWVKQRPGQGLEWIGWIFPRDGRT |
| | | HYNEKFKGKATLTVDTSSSTAYMELHSLTSEDSAVYFCSIYGSAMDLWGQGTSVTVSS |
| 394 | mice-53 | QVQLQQSGPELVKPGASVKLSCKASGYTFTNQDINWVKQRPGQGLEWIGWIFPRDGRT |
| | | HNNENFKGKATLTVDTSSSTAYMELHSLTAEDSAVYFCAIYGFAMDHWGQGTSVTVSS |
| 395 | mice-54 | EVQLQQSGPELVKPGASVKLSCKASGYTSTSYDINWVKQRPGQGLEWIGWIYPRDGRT |
| | | NYNEKFKGKATLTVDTSSSTAFMELHSLTSEDSAVYFCARYSAAMDYWGQGTSVTVSS |
| 396 | mice-55 | QVQLQQPGSELVRPGASVKIPCKTSGYSFTDFNINWVRQSHGKSLEWIGDINPDSGGTIY |
| | | NQKFKGKATLTVDKSSSTAYMELRSLTSEDTAVYYCARWGYYGGEGFDYWGQGTTLT |
| | | VSS |
| 397 | mice-56 | QIQLVQSGPELVKPGASVKLSCKASGYTFTSSDINWVKQRPGQGPEWIGWIYPRDGRTH |
| | | YNEKFKGKATLTVDTSSSTAYMELHSLTSEDSAVYFCARYAYALNSWGQGTSVTVSS |
| 398 | mice-57 | QVQLQQPGPELVKPGASVRVSCKASGYTFTRQDINWVKQRAGQGLEWIGWIYPRDGRT |
| | | EYNEKFKGKVTLTVDTSSSTAYMELHSLTSEDSAVYFCATYGSSLDNWGQGATLTVSS |
| 399 | mice-58 | QVQLQQSGPELVKPGASVKLSCKASGYTFTSYDINWVKQRPGQGLEWIGWIFPRDGDT |
| | | KYNEKFKGKATLTVDTSSSTAFMELHSLTSEDSAVYFCARYGSTLDVWGTGTTVTVSS |
| 400 | mice-59 | QIQLVQSGPELVMPGASVKLSCQASGYTFTNSDINWVKQRPGQGLEWIGWVYPGDGST |
| | | YYNEKFKGKATLTVDTSSSTAYMDLYSLTSEDSAVYFCARYAYALNSWGQGTSVTVSS |
| 401 | mice-60 | QIQLVQSGPELVKPGASVKLSCKASGYTFTNSDINWVKQRPGQGLEWIGWIYPRDGSTY |
| | | YNEKFKGKATLTVDTSSTTAYMEFHSLTSEDSAVYFCARYAYALNSWGQGTSVTVSS |
| 402 | mice-61 | QVQLQQSGPEVVKPGASVKLSCKASGYTFTSYDINWVKQRPGQGLEWIGWIFPRDGRT |
| | | EYNEKFKDKATLTVDTSSSTAYMELHSLTSEDSAFYFCARYGSSLDYWGQGTTLTVSS |
| 403 | mice-62 | QIQLVQSGPELVKPGASVKLSCQASGYTFTNSDINWVKQRPGQGLDWIGWVYPRDGST |
| | | YYNEKFKGKATLTVDTSSSTAYMELYSLTSEDSAVYFCARYAYALNYWGQGTSVTVSS |
| 404 | mice-63 | QIQLVQSGPELVKPGASVKLSCKASGYTFTSIDINWVKQRPGQGLEWIGWIYPRDGRTD |
| | | YNEKFKGKATLTVDTSSSTAYMELHSLTSEDSAVYFCARYTSSLDYWGQGTTLTVSS |
| 405 | mice-64 | QVQLQQSGPELVKPGASVKLSCKASGYTFTSIDINWVKQRPGQGLEWIGWIYPRDGRTN |
| | | YNEKFKGKATLTVDTSSSTAYMDLHSLTSEDSAVYFCARYTSSLDYWGQGTTLTVSS |
| 406 | mice-65 | QVQLQQSGPELVKPGASVKLSCRASGYTFTSYDINWVKQRPGQGLEWIGWNYPRDGRT |
| | | DYNEKFKGKATLTVDTSSNTAYMDLHSLTSEDSAVYFCTRYGSTLDVWGTGTSVTVSS |
| 407 | mice-66 | QIQLVQSGPELVKPGASVKLSCQASGYTFTNSDINWVKQRPGQGLEWIGWVYPRDGSA |
| | | YYNEKFKGKATLTVDTSSSTAYMELYSLTSEDSAVYFCARYAYALNFWGQGTSVTVSS |
| 408 | mice-67 | QVQLKQSGPELVKPGASVKVSCKASGYTFTRQDINWVKQRPGQGLEWIGWIYPRDGRT |
| | | EYNEKFKGKVTLTVDTSSSTAYMELHSLTSEDSAVYFCATYGSSLDNWGQGTTLTVSS |
| 409 | mice-68 | QVQLQQPGPELVKPGASVKLSCKASGYTFTNQDINWVKQRPGQGLEWIGWIFPRDGRT |
| | | HYNEKFKGKATLTVDTSSSTAYMELHSLTAEDSAVYFCAIYGSAMDQWGQGTSVTVSS |
| 410 | mice-69 | EVQLQQSGPELVKPGASVKLSCKASGYTFTNSDINWVKQRPGQGLEWIGWIYPRDGSTY |
| | | YNEKFKGKATLTVDTSSTTAYMEFHSLTSEDSAVYFCARYAYALNSWGQGTSVTVSS |
| 411 | mice-70 | QVQLQQPGAELMKPGASVKLSCKATGYTFTAYWLEWVKQRPGHGLEWIGEILPGSGY |
| | | AYYNAKFKGKATFTADISSNTAYMQLSGLTTEDSAIYYCARRGSNYPYWYFDVWGTGT |
| | | TVTVSS |
| 412 | mice-71 | QVQLQQSGAELMKPGASVKLSCKATGYTFTGYWLEWVKQRPGHGLEWIGETLPGSGY |
| | | NKYNEKFKGKATFTADTSSNTAHMQLSSLTTEDSAIYYCARRGSNYPYWYFDVWGTGT |
| | | TVTVSS |
| 413 | mice-72 | QVQLQQSGAELMKPGASVKLSCKATGYTFTGYWLEWVKQRPGHGLEWIGETLPGSGY |
| | | NKYNEKFKGKATFTADTSSNTAHMQLSSLTTEDSAIYYCARRGSNYPYWYFDVWGTGT |
| | | SVTVSS |
| 414 | mice-73 | QVQLQQSGPELVKPGASVKLSCRASGYTFTSYDINWVKQRPGQGLEWIGWNYPRDGRT |
| | | DYNEKFKGKATLTVDTSSNTAYMELHSLTSEDSAVYFCTRYGSTLDVWGTGTTVTVSS |
| 415 | mice-74 | QIQLVQSGPELVKPGASVKLSCRASGYTFTSYDINWVKQRPGQGLEWIGWNYPRDGRT |
| | | DYNEKFKGKATLTVDTSSNTAYMELHSLTSEDSAVYFCTRYGSTLDVWGTGTTVTVSS |
| 416 | mice-75 | QVQLQQPGPELVKPGASVKLSCRASGYTFTSYDINWVKQRPGQGLEWIGWIFPRDGRTE |
| | | YNAKFKGKATLTVDTSSNTAYMELHSLTSDDSAVYFCARYGSTLDVLGAGTTVTVSS |
| 417 | mice-76 | EVQLQQSGPELVKPGASVKLSCRASGYTFTSYDINWVKQRPGQGLEWIGWIFPRDGRTE |
| | | YNEKFKGKATLTVDTSSNTAYMELHSLTSEDSAVYFCARYGSTLDVWGTGTTVTVSS |
| 418 | mice-77 | QIQLVQSGPELVKPGASVKLSCRASGYTFTSYDINWVKQRPGQGLEWIGWIFPRDGRTE |
| | | YNEKFKGKVTLTVDTSSNTAYMELHSLTSEDSAVYFCARYGSTLDVWGPGTTVTVSS |
| 419 | mice-78 | QVQLQQFGPELVKPGSSVKLSCKASGYTFTDYDVNWVKQRPGQGLEWIGWNYPRDGR |
| | | TNYNEKFKGKATLTVDTSSSTAYMELHSLTSEDSAVYFCARYGSALNVWGTGTTVTVS |
| | | S |
| 420 | mice-79 | QVQLQQSGPELVKPGASVKLSCKASGYTFTDYDVNWVKQRPGQGLEWIGWNYPRDGR |
| | | TNYNEKFKGKATLTVDISSSTAYMELHSLTSEDSAVYFCARYGSALNVWGTGTTVTVSS |
| 421 | mice-80 | QIQLVQSGPELVKPGASVKLSCQASGYTFTNSDINWVKQRPGQGLEWIGWVYPRDGST |
| | | YYNEKFKGKATLTVDTSSSTAYMELYSLTSEDSAVYFCARYAYALNSWGQGTSVTVSS |
| 422 | mice-81 | QIQLVQSGPELVKPGASVKLSCQASGYTFTNSDINWVKQRPGQGLEWIGWVYPRDGST |
| | | YYNEKFKAKATLTVDTSSSTAYMELYSLTSEDSAVYFCARYAYALNYWGQGTSVTVSS |
| 423 | mice-82 | QIQLVQSGPELVKPGASVKLSCQASGYTFTNSDINWVKQRPGQGLEWIGWVYPRDGRT |
| | | YYNEKFKGKATLTVDTSSSTAYMELYSLTSEDSAVYFCARYAYALNSWGQGTSVTVSS |
| 424 | mice-83 | QIQLVQSGPELVKPGASVKLSCKASGYTFTNSDINWVKQRPGQGLEWIGWVYPRDGST |
| | | YYNEKFKGKATLTVDTSSSTAYMELHSLTSEDSAVYFCTRYAYALNSWGQGTSVTVSS |
| 425 | mice-84 | QIQLVQSGPELVKPGASVKLSCKASGYTFTNSDINWVKQRPGQGLEWIGWIYPRDGSTY |
| | | YNEKFKGKATLTVDTSSTTAYMEFHSLTSEDSAVYFCARYAYTLNSWGQGTSVTVSS |
| 426 | mice-85 | EVQLQQSGAELARPGASVKMSCKASGYTFTNSDINWVKQRPGQGLEWIGWIYPRDGST |
| | | YYNEKFKGKATLTVDTSSSTAYMELHSLTSEDSAVYFCTRYAYALNSWGQGTSVTVSS |
| 427 | mice-86 | QVQLQQSGPELVKPGASVKLSCKASGYTFTNSDINWVKQRPGQGLEWIGWIYPRDGST |
| | | YYNEKFKGKATLTVDTSSSTAYMELHSLTSEDSAVYFCTRYAYALNSWGQGTSVTVSS |
| 428 | mice-87 | EVQLQQSGPELVKPGASVKLSCQASGYTFTNSDINWVKQRPGQGLEWIGWVYPRDGRT |
| | | YYNEKFKGKATLTVDTSSSTAYMELYSLTSEDSAVYFCARYAYALNSWGQGTSVTVSS |
| 429 | mice-88 | EVQLQQSGPELVRPGASVKLSCKASGYSFTSGDINWVKQRPGQGLEWIAWIYPRDGSTE |
| | | YNEKFKGKATLTVDTSSSTAYMELHSLTSEDSAVYFCARYDYAFVYWGQGTLVTVSA |
| 430 | mice-89 | EVQLQQSGPELVRPGTSVRLSCKTSGYTFTSRDINWVKQRPGQGLEWIAWIYPRDGSTE |
| | | YNEKFKGKATLTVDTSSSTAYMELHSLTSEDSAVYFCTRYDYAFTYWGQGTLVTVSA |
| 431 | mice-90 | EVQLQQSGPEVVKPGASVKLSCKASGYTFTRNDINWVKQRPGQGLEWIGWIYPRDGSS |
| | | KNNEKFKGKATLTVDTSSSTAYMELHSLTSEDSAVYFCARYDYAFVYWGQGTTVTVSS |
| 432 | mice-91 | EVQLQQSGPELVKPGASVKLSCKASGYTFTSYDINWVKQRPGQGLEWIGWIYPRDGRT |
| | | KYSEKFKGKATLTVDTSSSTAYMELHSLTSEDSAVYFCARYGASMDYWGQGTSVTVSS |
| 433 | mice-92 | EVQLQQSGPELVKPGASVKLSCKASGYTFTSYDINWVKQSPGQGPEWIGWVYPRDGRT |
| | | QYNEKFKGKATLTVDTSSSTAYMELHSLTSEDSAVYFCARYGASMDYWGQGTSVTVSS |
| 434 | mice-93 | EVQLQQSGPELAKPGASVKLSCKASGYTFTSYDINWVKQRPGQGLEWIGWIYPRDGNT |
| | | NYNEKFKGKATLTVDTSSNTASMELHSLTSEDSAVYFCARYSAAMDYWGQGTSVTVSS |
| 435 | mice-94 | QVQLQQPGPELVKPGASVKLSCKASGYTSTSYDINWVKQRPGQGLEWIGWIYPRDGRT |
| | | NYNEKFKGKATLTVDTSSSTAFMELHSLTSEDSAVYFCARYSAAMDYWGQGTSVTVSS |
| 436 | mice-95 | QVQLQQSGPELVKPGASVKLSCKASGYTFTSYDINWVKQRPGQGLEWIGWIFPRDGRT |
| | | NYNEKFKDKATLTVDTSSSTAFMELHRLTSEDSAVYFCARYSAAMDYWGQGTSVTVSS |
| 437 | mice-96 | EVQLQQSGAELGKPGASVKLSCKASGYTFTNYDINWMKQRPGQGLEWIGWNYPRDGR |
| | | TKYNEKFKGKATLTVDTSSSTAYMELHSLTSEDSAVYFCARYGYAMDYWGQGTSVTV |
| | | SS |
| 438 | mice-97 | QVQLQQSGPDLVKPGASVKLSCKASGYIFASYDLNWVKQRPGQGLEWIGWIYPRDGRT |
| | | DYNEKFKGKATLTVDTSSSTAYMELHSLTSEDSAVYFCARYGYALDYWGQGTSVTVSS |
| 439 | mice-98 | QIQLVQSGPELVKPGASVKVSCKASGYTFTRQDINWVKQRPGQGLEWIGWIYPRDGRTE |
| | | YNEKFKGKATLTVDTSSSTAFMELHSLTSEDSAVYFCARYSAAMDYWGQGTSVTVSS |
| 440 | mice-99 | QIQLVQSGPELVKPGASVKLSCKASGYTFTNSDINWVKQRPGQGLEWIGWNYPRDGRT |
| | | KYNEKFKGKATFTVDTSSSTAYMDLQSLTSEDSAVYFCTRYGSAMDYWGQGTSVTVSS |
| 441 | mice-100 | EVQLQQSGPELVKPGASVKLSCKASGYTFTNQDINWVKQRPGQGLEWIGWIFPRDGRT |
| | | HYNENFKGKATLTVDTSSSTAYMELHSLTAEDSAVYFCAIYGIALDHWGQGTSVTVSS |
| 442 | mice-101 | QVQLQQSGPELVKPGASVKLSCKASGYTFTNQDINWVKQRPGQGLEWIGWIFPRDGRT |
| | | HYNENFKGKATLTVDTSSNTAYMELHSLTAEDSAVYFCAIYGIALDHWGQGTSVTVSS |
| 443 | mice-102 | QVQLQQSGPELVKPGASVKLSCKASGYTFTNQDINWVKQRPGQGLEWIGWIFPRDGRT |
| | | HYNENFKGKATLTVDTSSSTAYMELHSLTAEDSAVYFCAIYGIALDHWGQGTSVTVSS |
| 444 | mice-103 | QVQLQQSGPELVKPGASVKLSCKASGYTFTNQDINWVKQRPGQGLEWIGWIFPRDGRT |
| | | HYNENFKGKATLTVDTSSSTAYMELHSLTAEDSAVYFCAIYGAAMDHWGQGTSVTVSS |
| 445 | mice-104 | QVQLQQSGPELVKPGTSVKLSCKASGYTFTNQDINWVKQRPGQGLEWIGWIFPRDGRT |
| | | HYNENFKGKATLTVDTSSSTAYMELHSLTAEDSAVYFCSIYGIAMDHWGQGTSVTVSS |
| 446 | mice-105 | QVQLQQPGPELVKPGASVKLSCKASGYTFTNQDINWVKQRPGQGLEWIGWIFPRDGRT |
| | | HNNENFKGKATLTVDTSSSTAYMELHSLTAEDSAVYFCAIYGFAMDHWGQGTSVTVSS |
| 447 | mice-106 | QVQLQQSGPELVKPGASVKLSCKASGYTFTNQDINWVKQRPGQGLEWIGWIFPRDGRT |
| | | HYNENFKGKATLTVDTSSSTAYMELHSLTAEDSAVYFCAIYGFAMDHWGQGTSVTVSS |
| 448 | mice-107 | QVQLQQPGPELVKPGASVKLSCKASGYTFTNQDINWVKQRPGQGLEWIGWIFPRDGRT |
| | | HYNENFKGKATLTVDTSSSTAYMELHSLTAEDSAVYFCAIYGAAMDDWGQGTSVTVSS |
| 449 | mice-108 | QVQLQQPGPELVKPGASVKLSCKASGYTFTNQDINWVKQRPGQGLEWIGWIFPRDGRT |
| | | HYNENFKGKATLTVDTSSSTAFMELHSLTAEDSAVYFCAIYGIALDHWGQGTSVTVSS |
| 450 | mice-109 | EVQLQQSGPELVKPGASVKLSCKASGYTFTNQDINWVKQRPGQGLEWIGWIFPRDGRT |
| | | HYNENFKGKATLTVDTSSSTAYMELHSLTAEDSAVYFCAIYGFAMDHWGQGTSVTVSS |
| 451 | mice-110 | EVQLQQSGPELVKPGASVKLSCKASGYTFTNQDINWVKQRPGQGLEWIGWNFPRDGRT |
| | | HYNENFKGKATLTVDTSSSTAYMELHSLTAEDSAVFFCAIYGFALDHWGQGTSVTVSS |
| 452 | mice-111 | QVQLQQSGPELVKPGASVKLSCKASGYTFTNQDINWVKQRPGQGLEWIGWIFPRDGRT |
| | | HYNENFKGKATLTVDTSSSTAYMELHSLTAEDSAVYFCALYGFAMDQWGQGTSVTVSS |
| 453 | mice-112 | EVQLQQSGPELVKPGASVKLSCKASGYTFTNQDINWVKQRPGQGLEWIGWIFPRDGRT |
| | | HYNEKFKGKATLTVDTSSSTAYMELHSLTAEDSAVYFCAIYGSAMDQWGQGTSVTVSS |
| 454 | mice-113 | EVQLQQSGPELVKPGTSVKIPCKTSGYTFTDYNINWVKQSHGKSLEWIGDINPDSGGTV |
| | | YNQRFKDKATLTVDKSSSTAYMELRSLTSGDTAVYYCARWGYYDGRGFDYWGQGTTL |
| | | TVSS |
| 455 | mice-114 | EVQLQQSGAELVRPGASVTLSCKASGYTFTDYEIHWVKQIPVHGLEWIGVEDPETGGAA |
| | | YNQKFKGKAILTADKSSSTAYMELRSLTSEDSAVYYCTTPWLNYFEYWGQGTTLTVSS |
| 456 | mice-115 | QVQLQQSVAELVRPGASVKLSCTASGFNIEDTYMHWVKQRPEQGLEWIGRVDPASGNT |
| | | KYGPKFQGKATITADTSSNTAYLQLSSLSSADTAIYYCSTWGLFDYWGQGTTLTVSS |
| 457 | mice-116 | QVQLQQPGPELVKPGASVKLSCKASGYTFTSIDINWVKQRPGQGLEWIGWIYPRDGRTH |
| | | YNEKFKGKATLTVDTSSSTAYMELHSLTSEDSAVYFCARYTSSLDYWGQGTTLTVSS |
| 458 | mice-117 | QVQLQQSGPELVKPGASVKLSCKASGYTFTSIDINWVKQRPGQGLEWIGWIYPRDGRTD |
| | | YNEKFKGKATLTVDTSSSTAYMELHSLTSEDSAVYFCARYGSSLDYWGQGTTLTVSS |
| 459 | mice-118 | QVQLQQSGPELVKPGTSVRLSCKASGYTFTNSDINWVKQRPGQGLEWIGWIYPRDGRT |
| | | DYNEKFKGKATLTVDTSSSTAYMELHSLTSEDSAVYFCARYGSSLYWGQGTTLTVSS |
| 460 | mice-119 | EVQLQQSGPELVKPGASVKLSCKASGYTFTSSDINWVKQRPGQGPEWIGWIYPRDGRTH |
| | | YNEKFKGKATLTVDTSSSTAYMELHSLTSEDSAVYFCAPYGSSLAYWGQGTTLTVSS |
| 461 | mice-120 | QIQLVQSGPELVKPGASVKLSCKASGYTFTSSDINWVKQRPGQGLEWIGWIYPRDGSTH |
| | | YNENFKGKATLTVDTSSSTAYMELHSLTSEDSAVYFCAPYGSSLAYWGQGTTLTVSS |
| 462 | mice-121 | QVQLKESGPEVVKPGASVKLSCKASGYTFTSYDINWVKQRPGQGLEWIGWIYPRDGRT |
| | | EYNEKFKDKATLTVDTSSSTAYMELHSLTSEDSAVYFCARYGSSLDYWGQGTTLTVSS |
| 463 | mice-122 | QIQLVQSGPELVKPGASVKVSCKASGYTFTRQDINWVKQRPGQGLEWIGWIYPRDGSTK |
| | | YNEKFKGKVTLTVDTSSSTAYMELHSLTSEDSAVYFCATYGSSLDNWGQGTTLTVSS |
| 464 | mice-123 | QVQLQQSGPELVKPGASVKVSCKASGYTFTRQDINWVKQRPGQGLEWIGWIYPRDGST |
| | | KYNEKFKGKVTLTVDTSSSTAYMELHSLTSEDSAVYFCATYGSSLDNWGQGTTLTVSS |
| 465 | mice-124 | QIQLVQSGPELVKPGASVKVSCKASGYTFTRQDINWVKQRPGQGLEWIGWIYPRDGRTE |
| | | YNEKFKGKVTLTVDTSSSTAYMELHSLTPEDSAVYFCATYGSSLDNWGQGTTLTVSS |
| 466 | mice-125 | QVQLKESGPELVKPGASVKVSCKASGYTFTRQDINWVKQRPGQGLEWIGWIYPRDGRT |
| | | EYNEKFKGKVTLTVDTSSSTAYMELHSLTSEDSAVYFCATYGSSLDNWGQGTTLTVSS |
| 467 | mice-126 | QVQLQQSGAELAKPGASVKLSCKASGYTFTRQDINWVKQRPGQGLEWIGWIYPRDGRT |
| | | EYNEKFKGKVTLTVDTSSSTAYMELHSLTSEDSAVYFCATYGSSLDNWGQGTTLTVSS |
| 468 | mice-127 | QVQLQQPGPELVKPGASVKVSCKASGYTFTRQDINWVKQRPGQGLEWIGWIYPRDGRT |
| | | EYNEKFKGKVTLTVDTSSNTAYMELHSLTSEDSAVYFCATYGSSLDNWGQGTTLTVSS |
| 469 | mice-128 | QVQLQQSGPELVKPGASVKVSCKASGYTFTRQDINWVKQRPGQGLEWIGWIYPRDGRT |
| | | EYNEKFKGKVTLTVDTSSSTAYMELHSLTSEDSAVYFCATYGSSLDNWGQGTTLTVSS |
| 470 | mice-129 | EVQLQQSGPELVKPGASVKVSCKASGYTFTRQDINWAKQRPGQGLEWIGWIYPRDGRT |
| | | EYNEKFKGKVTLTVDTSSSTAYMELHSLTPEDSAVYFCATYGSSLDNWGQGTTLTVSS |
| 471 | mice-130 | EVQLQQSGPELVKPGASVKVSCKASGYTFTRQDINWVKQRPGQGLEWIGWIYPRDGRT |
| | | EYNEKFKGKVTLTVDTSSSTAYMELHSLTSEDSAVYFCATYGSSLDNWGQGTTLTVSS |
| 472 | mice-131 | EVQLQQSVAELVRPGASVKLSCTASGFNIENTYIHWVKQRPEQGLEWIGRIDPASGNTK |
| | | YAPKFQGKATITADTSSNTAYLQLSSLTSEDTAIYYCASTGPDYWGQGTTLTVSS |
| 473 | mice-132 | QIQLVQSGPELKKPGETVEISCKASGYTFTTHGMNWVKQAPGKGLKWMGWINTFSGVP |
| | | TYADDFRGRFDFFLATSASTAYLQINNLKNEDTATYFCARDYGTAYAMDSWGQGTSVT |
| | | VSS |
| 474 | mice-133 | EVKVEESGGGLVQPGGSMKLSCIASGFTFSNYWMNWVRQSPEKGLEWVAQIRLKSDNY |
| | | ATHYAESVKGNFTISRDDSKSSVYLQMNNLRAEDTGIYYCTPLLLRYRDYWGQGTTLT |
| | | VSS |
| 475 | mice-134 | EVQLQQSGPELVKPGASVKISCKASGYTFTDYYMNWVKQSHGKSLEWIGDVNPNSGGT |
| | | SQNQKFKGKATLTVDKSSTTAYMELRSLTSEDSAVYHCARGSNYVGGFTYWGQGTLV |
| | | AVSA |
| 476 | mice-135 | QVQLQQPGAELVKPGASVKLSCKASGYTFSNDWMHWVKQRPGRGLEWIGRIDPDSGG |
| | | TKYNEKFKNKATLTVDKPSSTVYMQLSSLTSEDSAVYYCARSEGYYGSRSYFDYWGQG |
| | | TTLTVSS |
| 477 | mice-136 | EFQLQQSGPELVKPGASVRISCKASGYTFTDYYLNWVKQSRGESLEWIGDINPNNGRTT |
| | | YNQKFTGKATWTVDKSSSTVYMELRSLTSDDSAVYYCAREGGYGWYFDVWGTGTTV |
| | | TVSS |
| 478 | mice-137 | DIQMTQTTSSLSASLGDRVTISCRASQDISNYLNWYQQKPDGTVKLLIYYTSRLHSGVPS |
| | | RFSGSGSGTDYSLTISNLEQEDIATYFCQQGNTLPFTFGSGTKLEIK |
| 479 | mice-138 | DIVLTQSPASLPVSLGQRATISCRASKSVSTSSYSYMHWYQQKPGQPPKLLIKYASNLES |
| | | GVPARFSGSGSGTDFTLNIHPVEEEDAATYYCQHSKEFPWTFGGGTKLEIK |
| 480 | mice-139 | DIKMTQSPSSMYASLGERVTITCKASQDINSFLSWFQQKPGKSPKTLIYRANRLVDGVPS |
| | | RFSGSGSGQDYSLTISSLEYEDMGIYYCLQCDEFPYTFGGGTKLEIK |
| 481 | mice-140 | DIKMTQSPSSMYASLGERVTITCKASQDINSYLSWFQQKPGKSPKTLIYRANRLVDGVPS |
| | | RFSASGSGQDYSLTISSLEYEDMGIYYCLQCDEFPYTFGGGTKLEIK |
| 482 | mice-141 | DIVMTQSHKFMSTSVGDRVSITCKASQDVSTAVAWYQQKPGQSPKLLIYWASARHTGV |
| | | PARFTGSGSGTDYTLTISSVQAEDLALYYCQQHYGTPPTFGGGTKLEIK |
| 483 | mice-142 | QIVLTQSPAIMSVSPGEKVTITCSATSRVTYMHWFQQKPGTAPKLWIYSTSNVASGVPAR |
| | | FSGSGSVTSYSLTISRMEAADAATYYCQQRSSYPPTFGGGTKLEIK |
| 484 | mice-143 | DIMLTQSPASLTVSLGQRATISCRASKSVSTSSYTYMHWYQQKPGQPPKLLIKYASNLES |
| | | GVPARFSGSGSGTDFILNIHPVEEEDAATYYCQHSREFPWTFGGGTKLEIK |
| 485 | mice-144 | DIVLTQSPASLAVSLGQRATISCRASKSVSTSSYNYMHWYQQKPGQPPKLLIKYASNLES |
| | | GVPARFSGSGSGTDFTLNIHPVEEEDAATYYCQHSREFPWTFGGGTKLEIK |
| 486 | mice-145 | QIVLTQSPALMSASPGEKVTMTCSASSSVSYMYWYQQKPRSSPKPWIYLTSNLASGVPA |
| | | RFSGSGSGTSYSLTISSMEAEDAATYYCQQWIIYPPMLTFGAGTKLELK |
| 487 | mice-146 | DIVLTQSPASLAVSLGQRATISCKASQSVSFAGISLMHWYQQKPGQQPKLLIYRASNLEA |
| | | GVPTRFSGSGSRTDFTLTINPVETDDVATYYCQQSNKDPTFGGGTKLELK |
| 488 | mice-147 | DIVMTQSHKFMSTSVGDRVSITCKASQDVSTAVAWYQQKPGQSPKLLIYSASYRYTGVP |
| | | DRFTGSGSGTDFTFTISSVQAEDLAVYYCHQHYSTPYTFGGGTKLEIKR |
| 489 | mice-148 | DVVMTQTPLSLPVSLGDQASISCRSSQNIVHSNGDTYLDWFLQKPGQSPKLLIYKVSNRF |
| | | SGVPDRFSGSGSGTDFTLKITRVESEDLGVYYCFQGSHDPYTFGGGTKLEIKR |
| 490 | mice-149 | ENVLTQSPAIMSASPGERVTMTCSASSSVSYMHWYQQKSSTSPKLWISDTSKLASGVPG |
| | | RFSGSGSGNSYSLTISSMEAEDVATYYCFQESGYPFTFGAGTKLELK |
| 491 | mice-150 | DVVMTQTPLSLPVSLGDQASISCRSSQSIVHSNGDTYLEWYLQKPGQSPKLLIYKVSNRF |
| | | SGVPDRFSGSGSGTDFTLKISRVEAEDLGVYYCFQGSHVPYTFGGGTKLEIKR |
| 492 | mice-151 | ENVLTQSPAIMSASPGEKVTMTCSASSSVSYMHWYQQKSSTSPKLWIYDTSKLASGVPG |
| | | RFSGSGSGNSYSLTISSMEAEDVATYYCFQESGYPLTFGAGTKLELK |
| 493 | mice-152 | EILLTQSPAIIVASPGEKVTITCSATSSVIYMNWYQQKPGSSPKIWIYGISNLASGVPARFS |
| | | GSGSGTSFSFTINSMEAEDVATYYCQQRSSYPFTFGSGTKLEIK |
| 494 | mice-153 | DVQITQSPSYLAASPGETITMNCRASKSIRKYLAWYQEKPGKSNKILIYSGSTLQSGIPSRF |
| | | SGSGSGTDFTLTISSLEPEDFAMYYCQQHNEYPLTFGAGTKLELK |
| 495 | mice-154 | QIVLTQSPAIMSASPGEKVTMTCSASSSVSYMHWYQQKSGTSLKRWIYDTSKLASGVPA |
| | | RFSGSGSGTSYSLTVSSMEAEDAATYYCQQWTSTPYTFGGGTKLEIK |
| 496 | mice-155 | QIVLTQSPAIMSASPGEKVTMTCSASSSVSYMHWYQQKSGTSPKRWIYDTSKLASGVPA |
| | | RFSGSGSGTSYSLTISTMEAEDAATYYCQQWTSTPYTFGGGTKLEIK |
| 497 | mice-156 | DIQMTQTTSSLSASLGDRVTISCRASQDITNYLNWYQQKPDGTVKLLIYSTSRLHSGVPS |
| | | RFSGSGSGTDYSLTISNLEQEDIATYFCQQGNTLPLTFGAGTKLELK |
| 498 | mice-157 | DIKMTQSPSSMYASLGERVTITCKASQDINSYLSWFQQKPGKSPKTLIYRANRLLDGVPS |
| | | RFSGSGSGQDYSLTISSLEYEDMGIYYCLQYDEFPYTFGGGTKLEIK |
| 499 | mice-158 | DIVMTQPQKFMPTSVGDRVSVTCKASQNVGTNVAWYQQKAGQSPKVLIYSASYRYSG |
| | | VPDRFTGSGSGTAFTLTISNVQSEDLAEYFCHQYNNFPFSFGSGTMLEIK |
| 500 | mice-159 | DIKMTQSPSSMYASLGDRVTITCKASQDIDSYLSWFQQKPGKSPKTLMYHTNRLIVGVPS |
| | | RFSGTGSGQDYALTISSLEYEDLGIYYCQQYDEFPSTFGSGTKLEIK |
| 501 | mice-160 | DIQMTQTTSSLSASLGDRVTISCRASQDISNYLNWYQQKPDGTVKLLIYYTSRLHSGVPS |
| | | RFSGSGSGTDYSLTISNLDQEDIATYFCQQGNTLPLTFGAGTKLELK |
| 502 | mice-161 | DNVMTQSQKFMSTSVGDRVSVTCKASQNVGTNVAWYQQKAGQSPKVLIYSASYRYSG |
| | | VPDRFTGSGSGTAFTLTISNVQSEDLAEYFCQQYNSFPFTFGSGTKLEIK |
| 503 | mice-162 | DVVMTQTPLTLSVTIGQPASISCKSSQSLLDSDGKTYLSWLLQRPGQSPKRLIILVSKLDS |
| | | GVPDRFTGSGSGTDFTLKISRVEAEDLGVYYCWQGTHFPQTFGGGTKLEIK |
| 504 | mice-163 | DVLMTQAPLSLPVSLGDQASISCRSSQSIVHSNGDTYLDWYLQKPGQSLKLLIYKLSNRF |
| | | SGVPDRFSGSGSGTDFTLKISRVEAEDLGVYHCFQGSHVPYTFGGGTKLEIK |
| 505 | mice-164 | DVVMTQTPLSLPVSLGDQASISCRSSQSIVHGNGNTYLEWYLQKPGQSPKLLIYKVSNRF |
| | | SGVPDRFSGSGSGTDFTLKISRVEAEDLGVYYCFQGSHVPYTFGGGTKLELKR |
| 506 | mice-165 | DVVMTQTPLSLPVSLGDQASISCRSSQSIVHSNGNTYLEWYLQKPGQSPKLLIYKVSNRF |
| | | SGVPDRFSGSGSGTDFTLKISRVEAEDLGVYYCFQGSHVPYTFGGGTKLELKR |
| 507 | mice-166 | DVVMTQTPLSLPVSLGDQASISCRSSQSIVHSNGSTYLEWYLQKPGQSPKLLIYKVSNRF |
| | | SGVPDRFSGSGSGTDFTLKISRVEAEDLGVYYCFQGSHVPYTFGGGTKLEIK |
| 508 | mice-167 | DVVMTQTPLSLPVSLGDQASISCRSSQNIVHSNGDTYLDWFLQKPGQSPQLLIYKVSNRF |
| | | SGVPDRFSGSGSGTDFTLKISRVEAEDLGIYYCFQGSHVPYTFGGGTKLEIK |
| 509 | mice-168 | DVVMTQTPLSLPVSLGDQASISCRSSQSIVHSNGHTYLEWYLQKPGQSPKLLIYKVSNRF |
| | | SGVPDRFSGSGSGTDFTLKISRVEAEDLGVYYCFQGSHVPYTFGGGTKLEIK |
| 510 | mice-169 | DVVMTQTPLSLPVSLGDQASISCRPSQTIVHSNGDTYLDWFLQKPGQSPKLLIYKVSNRF |
| | | SGVPDRFSGSGSGTDFTLKISRVEAEDLGVYYCFQGSHVPYTFGGGTRLEIK |
| 511 | mice-170 | DVVMTQTPLSLPVSLGDQASISCRSSQSIVHSNGNTYLEWYLQKPGQSPKLLIYKVSNRF |
| | | SGVPDRFSGSGSGTDFTLKISRVEAEDLGVYYCFQGSHVPLTFGDGTKLEIK |
| 512 | mice-171 | DVVMTQTPLSLPVSLGDQASISCRSSQSIVHSNGDTYLDWFLQKPGQSPKLLIYKVSNRF |
| | | SGVPDRFSGSGSGTDFTLKITRVESEDLGVYYCFQGSHDPYTFGGGTKLEIK |
| 513 | mice-172 | DVVMTQTPLSLPVSLGDQASISCRSSQSVVHNNGNTYLEWYLQKPGQSPKLLIYKVSNR |
| | | FSGVPDRFSGSGSGTDFTLKISRVEAEDLGVYYCFQGSHVPYTFGGGTKLELK |
| 514 | mice-173 | DVVMTQTPLSLPVSLGDQASISCRSSQSIVHSNGDTYLEWYLQKPGQSPKLLIYKVSNRF |
| | | SGVPDRFSGSGSGTDFTLKISRVEAEDLGVYYCFQGSHVPYTFGGGTKLEIK |
| 515 | mice-174 | DVVMTQTPLSLPVSLGDQASISCRSSQSIVHTNGDTYLDWYLQKPGQSPKLLIYKVSNRF |
| | | SGVPDRFSGSGSGTDFTLKISRVEAEDLGVYYCFQGSHVPYTFGGGTKLEIK |
| 516 | mice-175 | DVVMTQTPLSLPVSLGDQASISCRSSQSIVHDNGDTYLEWYLQKPGQSPKLLIYKVSNRF |
| | | SGVPDRFSGSGSGTDFTLKINRVEAEDLGVYYCFQGSHVPYTFGGGTKLEIK |
| 517 | mice-176 | DVVMTQTPLSLPVSLGDQASISCRSSQNIVHSNGNTYLEWYLQKPGQSPKLLIYKVSNRF |
| | | SGVPDRFSGSGSGTDFTLKISRVEAEDLGVYYCFQGSHVPYTFGGGTKLEIK |
| 518 | mice-177 | DIVLTQSPASLAVSLGQRATISCRASESVDDYGISFMHWYQQKPGQPPKLLIYRASNLES |
| | | GIPARFSGSGSRTDFTLTIDPVETDDVATYYCQQSYEDPPTFGAGTKLEIK |
| 519 | mice-178 | DIVLTQSPASLAVSLGQRATISCRASESVDNYGISFMHWYQQKPGQPPKLLVYRASNLES |
| | | GIPARFSGSGSRTDFTLTINPVETDDVATYYCQQSNKDPPTFGGGTKLEIK |
| 520 | mice-179 | DIVLTQSPASLAVSLGQRATISCRASESVDNYGISFMHWYQQKPGQPPKLLIYRASNLES |
| | | GIPARFSGSGSRTDFTLTINPVETDDVATYYCQQSNKDPPTFGAGTKLELK |
| 521 | mice-180 | DIVLTQSPASLAVSLGQRATISCRASKSVSTSSYSYMHWYQQKPGQPPKLLIKYASNLES |
| | | GVPARFSGSGSGTDFTLNIHPVEEEDAATYYCQHSREFPTFGGGTKLEIK |
| 522 | mice-181 | DVVMTQTPLSLPVSLGDQASISCRSSQSIVHSNGDTYLDWYLEKPGQSPKLLIYKVSNRF |
| | | SGVPDRFSGSGSGTDFTLKISRVEAEDLGVYYCFQGSHVPYTFGGGTKLEIKR |
| 523 | mice-182 | DVVMTQTPLSLPVSLGDQASISCRSSQSIVHNNGDTYLDWYLQKPGQSPKLLIYKVSNRF |
| | | SGVPDRFSGSGSGTDFTLKISRVEAEDLGVYYCFQGSHVPYTFGGGTKLELK |
| 524 | mice-183 | DVVMTQTPLSLPVSLGDQASISCRSSQSIVHGNGNTYLEWYLQKPGQSPKLLIYKVSNRF |
| | | SGVPDRFSGSGSGTDFTLKISRVEAEDLGVYYCFQGSHVPYTFGGGTKLEIK |
| 525 | mice-184 | DVVMTQTPLSLPVSLGDQASISCRSSQNIIHSNGDTYLDWFLQKPGQSPKLLIYKVSNRFS |
| | | GVPDRFSGSGSGTDFTLKISRVEAEDLGVYYCFQGSHVPYTFGGGTKLEIK |
| 526 | mice-185 | DVVMTQTPLSLPVSLGDQASISCRSSQSIVHSNGNTYLEWYLQKPGQSPKLLIYKVSNRF |
| | | SGVPDRFSGSGSGTDFTLKISRVEAEDLGVYYCFQGSHVPYTFGRGDQAGNKT |
| 527 | mice-186 | DVVMTQTPLSLPVSLGDQASISCRSSQSIVHSNGDTYLEWYLQKPGQSPKLLIYKVSNRF |
| | | SGVPDRFSGSGSGTDFTLKISRVEAEDLGIYYCFQGSHVPYTFGGGTKLEIK |
| 528 | mice-187 | DVVMTQTPLSLPVSLGNQASISCRSSQDIVHTNGDTYLDWYLQKPGQSPKLLIYKVSNRF |
| | | SGVPDRFSGSGSGTDFTLKINRVEAEDLGVYYCFQGSHVPYTFGGGTKLELK |
| 529 | mice-188 | DVVMTQTPLSLPVSLGDQASISCRSSQSIVHSNGDTYLDWFLQKPGQSPKLLIYKVSNRF |
| | | SGVPDRFSGSGSGTDFTLKISRVEAEDLGVYYCFQGSHVPYTFGGGTKLELK |
| 530 | mice-189 | DVVMTQTPLYLPVSLGDQASISCRSSQSIVHSNGDTYLDWFLQKPGQSPKLLIYKVSNRF |
| | | SGVPDRFSGSGSGTDFTLKISRVEAEDLGVYYCFQGSHVPYTFGGGTKLELK |
| 531 | mice-190 | DVVMTQTPLSLPVSLGDQASISCRSSQTIVHSNGDTYLDWYLQKPGQSPKLLISKVSNRF |
| | | SGVPDRFSGSGSGTDFTLKISSVEAEDLGVYYCFQGSHVPYTFGGGTKLEIK |
| 532 | mice-191 | DVVMTQTPLSLPVSLADQASISCRSSQSIVHDNGDTYLEWYLQKPGQSPKLLIYKVSNRF |
| | | SGVPDRFSGSGSGTDFTLKISRVEAEDLGVYYCFQGSHVPYTFGGGTKLEIK |
| 533 | mice-192 | DVVMTQTPLSLPVSLGDQASISCRSSQSIVHNNGDTYLDWYLEKPGQSPKLLIYKVSNRF |
| | | SGVPDRFSGSGSGTDFTLKISRVEAEDLGVYYCFQGSHVPYTFGGGTKLEIK |
| 534 | mice-193 | DVVMTQTPLSLPVSLGDQASISCRSSQNIVHNNGDTYLDWFLQKPGQSPKLLIYKVSNRF |
| | | SGVPDRFSGSGSGTDFTLKISRVEAEDLGVYYCFQGSHVPYTFGGGTKLELK |
| 535 | mice-194 | DVVMTQTPLSLPVSLGDQASISCKSSQSIVHSNGNTYLEWYLQKPGQSPKLLIYKVSNRF |
| | | SGVPDRFSGSGSGTDFTLKISRVEAEDLGVYYCFQGSHVPYTFGGGTKLEIK |
| 536 | mice-195 | DVVMTQTPLSLPVSLGDQASISCRSSQNIVHSNGDTYLEWYLQKPGQSPKLLIYKVSNRF |
| | | SGVPDRFSGSGSGTDFTLKISRVEAEDLGVYYCFQGSYVPYTFGGGTKLEIK |
| 537 | mice-196 | DVVMTQTPLSLPVSLGDQASISCRSSQNIVHSNGDTYLEWYLQKPGQSPKLLIYKVSNRF |
| | | SGVPDRFSGSGSGTDFTLKISRVEAEDLGVYYCFQGSHVPYTFGGGTKLEIK |
| 538 | mice-197 | DVVMTQTPLSLPVSLGDQASISCRSSQNIVHSNGDTYLDWFLQKPGQSPKLLIYKVSNRF |
| | | SGVPDRFSGSGSGTDFTLKISRVEAEDLGVYYCFQGSHVPYTFGGGTKLEIK |
| 539 | mice-198 | DVVMTQTPLSLPVSLGDQASISCRSSQSIVHSNGDTYLDWFLQKPGQSPKLLIYKVSNRF |
| | | SGVPDRFSGSGSGTDFTLKISRVESEDLGVYYCFQGSHDPYTFGGGTKLEIK |
| 540 | mice-199 | DVVMTQTPLSLPVSLGDQASISCRSSQSIVHSNGNTYLEWYLQKPGQSPKLLIYKVSDRF |
| | | SGVPDRFSGSGSGTDFTLKISRVEAEDLGVYYCFQGSHVPYTFGGGTKLEIK |
| 541 | mice-200 | DVVMTQTPLSLPVSLGHQASISCRSSQSIVHSNGNTYLEWYLQKPGQSPKLLIYKVSNRF |
| | | SGVPDRFSGSGSGTDFTLKISRVEAEDLGVYYCFQGSHVPYTFGGGTKLELK |
| 542 | mice-201 | DVVMTQTPLSLPVSLADQASISCRSSQSIVHDNGDTYLEWYLQKPGQSPKLLIYKVSNRF |
| | | SGVPDRFSGSGSGTDFTLKISRVEAEDLGVYYCFQGSHVPYTFGWRDQAGNKT |
| 543 | mice-202 | DVVMTQTPLSLPVSLGDQASISCRSSQSIVHSNGNTYLEWYLQKPGQSPKLLIYKASNRF |
| | | SGVPDRFSGSGSGTDFTLRISSVEAEDLGVYYCFQGSHVPYTFGGGTKLEIK |
| 544 | mice-203 | DVVMTQTPLSLPVSLGDQASISCRSSQSIVHSNGNTYLEWYLQKPGQSPKLLIYKVSNRF |
| | | SGVPDRFSGSGSGTDFTLKISRVEAEDLGVYYCFQGSHVPLTFGAGTKLELK |
| 545 | mice-204 | DVVMTQTPLSLPVSLGDQASISCRSSQSLVHSNGNTYLHWYLQKPGQSPKLLIYKVSNR |
| | | FSGVPDRFSGSGSGTDFTLKISRVEAEDLGVYFCSQSTHVPFTFGSGTKLEIK |
| 546 | mice-205 | EIVLTQSPALMSASPGEKVTITCSASSSVSYMHWFQQKPGTSPKLWIYSTSNLASGVPAR |
| | | FSGSGSGTSYSLTISRMEAEDAATYYCQQRSSYPFTFGSGTKLEIK |
| 547 | mice-206 | DVVMTQTPLSLPVSLGDQASISCRSSQSIVHSNGNTYLEWYLQKPGQSPKLLIYKVSNRF |
| | | SGVPDRFSGSGSGTDFTLKISRVEAEDLGVYYCFQGSHVPYTFGGGTKLEIKR |
| 548 | mice-207 | DVVMTQTPLSLPVSLGDQASISCRSSQNIVHSNGDTYLDWYLQKPGQSPKLLISKVSNRF |
| | | SGVPDRFSGSGSGTDFTLKISRVEAEDLGVYYCFQGSHVPYTFGGGTKLEIK |
| 549 | mice-208 | DVVMTQTPLSLPVSLGDQASISCRSSQNIVHSNGDTYLDWFLQKPGQSPKLLISKVSNRF |
| | | SGVPDRFSGSGSGTDFTLKISRVEAEDLGVYYCFQGSHVPYTFGGGTKLEIK |
| 550 | mice-209 | DVVMTQTPLSLPVSLGDQASISCRSSQSIVHSNGDTYLDWFLQKPGQSPKLLIYKVSNRF |
| | | SGVPDRFSGSGSGTDFTLKISRVEAEDLGVYYCFQGSHVPYTFGGGTKLEIK |
| 551 | mice-210 | DVVMTQTPLSLPVSLGDQASISCRSSQNIVHSNGDTYLDWFLQKPGQSPKLLIYKVSNRF |
| | | SGVPDRFSGSGSGTDFTLKISRVEAEDLGVYYCFQGSHVPYTFGGGTKLELK |
| 552 | mice-211 | DVVMTQTPLSLPVSLADQASISCRSSQSIVHDNGDTYLEWYLQKPGQSPRLLIYKVSNRF |
| | | SGVPDRFSGSGSGTDFTLKINRVEAEDLGVYYCFQGSHVPYTFGGGTKLEIK |
| 553 | mice-212 | DVVMTQTPLSLPVSLGDQASISCRSSQSIVHSNGNTYLEWYLQKPGQSPKLLIYKVSNRF |
| | | SGVPDRFSGSGSGTDFTLKISRVEAEDLGVYYCFQGSHVPYTFGGGTKLEIK |
| 554 | mice-213 | DVVMTQTPLSLPVSLGDQASISCRSSQSIVHNNGDTYLEWYLQKPGQSPKLLIYKVSNRF |
| | | SGVPDRFSGSGSGTDFTLKISRVEAEDLGVYYCFQGSHVPYTFGGGTKLEIK |
| 555 | mice-214 | DVVMTQTPLSLPVSLGDQASISCRSSQNIVHTNGDTYLDWYLQKPGQSPKLLIYKVSNRF |
| | | SGVPDRFSGSGSGTDFTLKISRVEAEDLGVYYCFQGSHVPYTFGGGTKLELK |
| 556 | mice-215 | DVVMTQTPLSLPVSLGDQASISCRSSQTIVHSNGNTYLDWYLQKPGHSPQLLIYKVSNRF |
| | | SGVPDRFSGSGSGTDFTLKISRVEAEDLGVYYCFQGSHVPYTFGGGTKLEIK |
| 557 | mice-216 | DVVMTQTPLSLPVSLGDQASISCRSSQSIVHSNGNTYLEWYLQKPGQSPKLLIYKVSNRF |
| | | SGVPDRFSGSGSGTDFTLKISRVEAEDLGVYYCFQGSHVPYTFGSGTKLEIK |
| 558 | mice-217 | DVVMTQTPLSLPVSLGDQASISCRSSQSIVHSNGNTYLEWYLQKPGQSPKLLIYKVSNRF |
| | | SGVPDRFSGSGSGTDFTLKISRVEAEDLGVYYCFQGSHVPYTFGGGTKLELK |
| 559 | mice-218 | DVVMTQTPLSLPVSLGDQASISCRSSQNIVHSNGDTYLEWYLQKPGQSPKLLIYKVSSRF |
| | | SGVPDRFSGSGSGTDFTLKISRVEAEDLGIYYCFQGSHVPYTFGGGTKLEIK |
| 560 | mice-219 | EILLTQSPAIIAASPGEKVTITCSASSSVSYMNWYQQKPGSSPKIWIYGISNLASGVPARFS |
| | | GSGSGTSFSFTINSMEAEDVATYYCQQRSSYPFTFGSGTKLEIK |
| 561 | mice-220 | DVLMTQAPLSLPVSLGDQASISCRSNQTIVHSNGNTYLEWYLQKPGQSPKLLIYKVSNRF |
| | | SGVPDRFSGSGSGTDFTLKISGVEAEDLGVYYCFQGSHVPLTFGAGTKLELN |
| 562 | mice-221 | ENVLTQSPAIMSASPGEKVTMTCSASSSVSYMHWYQQKSSTSPKLWIYDTSNLASGVPG |
| | | RFSGSGSGNSYSLTISSMEAEDVATYYCFQENGHPFTFGGGTKLEMK |
| 563 | mice-222 | DILMTQSPSSMSVSLGDTVSITCHASQDIFSYIGWLQQKPGKSFKGLIHHGTNLEDGVPSR |
| | | FRGNGSGADYSLTISSLESEDFADYYCVQYAQFPYTFGGGTKLEIK |
| 564 | mice-223 | DIVMTQSPSSLRVSAGEKVTMSCKSSQSLLNSGNQKNYLAWYQQKPGQPPKLLIYGAST |
| | | RESGVPDRFTGSGSGTDFTLTISSVQAEDLAVYYCQNDHSYPYTFGGGTKLEIK |
| 565 | mice-224 | EIVLTQSPTTMAASPGEKITITCSTSSSIISNYSHWYQQKPGFSPKLLIYKTSNLPSGVPARF |
| | | SGSGSGTSYSLTIGTMEAEDAATYYCQQGSDMPITFGAGTKLELK |

1. An isolated antigen-binding protein specifically binding to TNF-like protein A (TL1A), comprising a heavy chain variable region (VH) including heavy chain complementarity determining regions (HCDRs) 1-3 and a light chain variable region (VL) including light chain complementarity determining regions (LCDRs) 1-3, wherein
the VH comprises HCDR1 as set forth in SEQ ID NO: 1, HCDR2 as set forth in SEQ ID NO: 5, HCDR3 as set forth in SEQ ID NO: 14, and the VL comprises LCDR1 as set forth in SEQ ID NO: 42, LCDR2 as set forth in SEQ ID NO: 43, and LCDR3 as set forth in SEQ ID NO: 44: or, the VH comprises HCDR1 as set forth in SEQ ID NO: 107, HCDR2 as set forth in SEQ ID NO: 115, HCDR3 as set forth in SEQ ID NO: 120, and the VL comprises LCDR1 as set forth in SEQ ID NO: 154, LCDR2 as set forth in SEQ ID NO: 159, and LCDR3 as set forth in SEQ ID NO: 163: or the VH comprises HCDR1 as set forth in SEQ ID NO: 235, HCDR2 as set forth in SEQ ID NO: 238, HCDR3 as set forth in SEQ ID NO: 242, and the VL comprises LCDR1 as set forth in SEQ ID NO: 269, LCDR2 as set forth in SEQ ID NO: 274, and LCDR3 as set forth in SEQ ID NO: 277.

2. The isolated antigen-binding protein according to claim 1, wherein the VH comprises
HCDR1 as set forth in SEQ ID NO: 2, HCDR2 as set forth in SEQ ID NO: 6, and HCDR3 as set forth in SEQ ID NO: 15: or,
HCDR1 as set forth in SEQ ID NO: 2, HCDR2 as set forth in SEQ ID NO: 7, and HCDR3 as set forth in SEQ ID NO: 15: or,
HCDR1 as set forth in SEQ ID NO: 2, HCDR2 as set forth in SEQ ID NO: 8, and HCDR3 as set forth in SEQ ID NO: 15: or,
HCDR1 as set forth in SEQ ID NO: 3, HCDR2 as set forth in SEQ ID NO: 6, and HCDR3 as set forth in SEQ ID NO: 15: or,
HCDR1 as set forth in SEQ ID NO: 2, HCDR2 as set forth in SEQ ID NO: 6, and HCDR3 as set forth in SEQ ID NO: 16: or,
HCDR1 as set forth in SEQ ID NO: 2, HCDR2 as set forth in SEQ ID NO: 6, and HCDR3 as set forth in SEQ ID NO: 17: or,
HCDR1 as set forth in SEQ ID NO: 2, HCDR2 as set forth in SEQ ID NO: 6, and HCDR3 as set forth in SEQ ID NO: 18: or,
HCDR1 as set forth in SEQ ID NO: 4, HCDR2 as set forth in SEQ ID NO: 6, and HCDR3 as set forth in SEQ ID NO: 15: or,
HCDR1 as set forth in SEQ ID NO: 2, HCDR2 as set forth in SEQ ID NO: 9, and HCDR3 as set forth in SEQ ID NO: 15: or,
HCDR1 as set forth in SEQ ID NO: 2, HCDR2 as set forth in SEQ ID NO: 10, and HCDR3 as set forth in SEQ ID NO: 15: or,
HCDR1 as set forth in SEQ ID NO: 2, HCDR2 as set forth in SEQ ID NO: 11, and HCDR3 as set forth in SEQ ID NO: 15: or,
HCDR1 as set forth in SEQ ID NO: 2, HCDR2 as set forth in SEQ ID NO: 12, and HCDR3 as set forth in SEQ ID NO: 15: or,
HCDR 1 as set forth in SEQ ID NO: 2, HCDR2 as set forth in SEQ ID NO: 13, and HCDR3 as set forth in SEQ ID NO: 15: or,
HCDR1 as set forth in SEQ ID NO: 3, HCDR2 as set forth in SEQ ID NO: 6, and HCDR3 as set forth in SEQ ID NO: 16: or,
HCDR1 as set forth in SEQ ID NO: 3, HCDR2 as set forth in SEQ ID NO: 6, and HCDR3 as set forth in SEQ ID NO: 17: or,
HCDR1 as set forth in SEQ ID NO: 3, HCDR2 as set forth in SEQ ID NO: 9, and HCDR3 as set forth in SEQ ID NO: 15: or,
HCDR1 as set forth in SEQ ID NO: 3, HCDR2 as set forth in SEQ ID NO: 10, and HCDR3 as set forth in SEQ ID NO: 15: or,
HCDR1 as set forth in SEQ ID NO: 3, HCDR2 as set forth in SEQ ID NO: 11, and HCDR3 as set forth in SEQ ID NO: 15: or,
HCDR1 as set forth in SEQ ID NO: 2, HCDR2 as set forth in SEQ ID NO: 9, and HCDR3 as set forth in SEQ ID NO: 16; or,
HCDR1 as set forth in SEQ ID NO: 2, HCDR2 as set forth in SEQ ID NO: 10, and HCDR3 as set forth in SEQ ID NO: 16: or,
HCDR1 as set forth in SEQ ID NO: 2, HCDR2 as set forth in SEQ ID NO: 11, and HCDR3 as set forth in SEQ ID NO: 16; or,
HCDR1 as set forth in SEQ ID NO: 2, HCDR2 as set forth in SEQ ID NO: 12, and HCDR3 as set forth in SEQ ID NO: 16; or,
HCDR1 as set forth in SEQ ID NO: 2, HCDR2 as set forth in SEQ ID NO: 10, and HCDR3 as set forth in SEQ ID NO: 17.

3. The isolated antigen-binding protein according to claim 2, wherein the VH comprises HCDR 1 as set forth in SEQ ID NO: 2, HCDR2 as set forth in SEQ ID NO: 6, and HCDR3 as set forth in SEQ ID NO: 15, and the VL comprises LCDR1 as set forth in SEQ ID NO: 42, LCDR2 as set forth in SEQ ID NO: 43, and LCDR3 as set forth in SEQ ID NO: 44.

4. The isolated antigen-binding protein according to claim 1, wherein
the VH comprises a sequence as set forth in SEQ ID NO: 62, and the VL comprises a sequence as set forth in SEQ ID NO: 102; or,
the VH comprises a sequence as set forth in SEQ ID NO: 63, and the VL comprises a sequence as set forth in SEQ ID NO: 103: or,
the VH comprises a sequence as set forth in SEQ ID NO: 63, and the VL comprises a sequence as set forth in SEQ ID NO: 106; or,
the VH comprises a sequence as set forth in SEQ ID NO: 64, and the VL comprises a sequence as set forth in SEQ ID NO: 103: or,
the VH comprises a sequence as set forth in SEQ ID NO: 64, and the VL comprises a sequence as set forth in SEQ ID NO: 106; or,
the VH comprises a sequence as set forth in SEQ ID NO: 65, and the VL comprises a sequence as set forth in SEQ ID NO: 103: or,
the VH comprises a sequence as set forth in SEQ ID NO: 65, and the VL comprises a sequence as set forth in SEQ ID NO: 106; or,
the VH comprises a sequence as set forth in SEQ ID NO: 66, and the VL comprises a sequence as set forth in SEQ ID NO: 106; or,
the VH comprises a sequence as set forth in SEQ ID NO: 67, and the VL comprises a sequence as set forth in SEQ ID NO: 106; or,
the VH comprises a sequence as set forth in SEQ ID NO: 68, and the VL comprises a sequence as set forth in SEQ ID NO: 106; or,
the VH comprises a sequence as set forth in SEQ ID NO: 69, and the VL comprises a sequence as set forth in SEQ ID NO: 106; or,
the VH comprises a sequence as set forth in SEQ ID NO: 70, and the VL comprises a sequence as set forth in SEQ ID NO: 106; or,
the VH comprises a sequence as set forth in SEQ ID NO: 70, and the VL comprises a sequence as set forth in SEQ ID NO: 104: or,
the VH comprises a sequence as set forth in SEQ ID NO: 70, and the VL comprises a sequence as set forth in SEQ ID NO: 105: or,
the VH comprises a sequence as set forth in SEQ ID NO: 66, and the VL comprises a sequence as set forth in SEQ ID NO: 104; or,
the VH comprises a sequence as set forth in SEQ ID NO: 67, and the VL comprises a sequence as set forth in SEQ ID NO: 104: or,
the VH comprises a sequence as set forth in SEQ ID NO: 66, and the VL comprises a sequence as set forth in SEQ ID NO: 105: or,
the VH comprises a sequence as set forth in SEQ ID NO: 71, and the VL comprises a sequence as set forth in SEQ ID NO: 106; or,
the VH comprises a sequence as set forth in SEQ ID NO: 72, and the VL comprises a sequence as set forth in SEQ ID NO: 106; or,
the VH comprises a sequence as set forth in SEQ ID NO: 73, and the VL comprises a sequence as set forth in SEQ ID NO: 106; or,
the VH comprises a sequence as set forth in SEQ ID NO: 74, and the VL comprises a sequence as set forth in SEQ ID NO: 106; or,
the VH comprises a sequence as set forth in SEQ ID NO: 75, and the VL comprises a sequence as set forth in SEQ ID NO: 106; or,
the VH comprises a sequence as set forth in SEQ ID NO: 76, and the VL comprises a sequence as set forth in SEQ ID NO: 106; or,
the VH comprises a sequence as set forth in SEQ ID NO: 77, and the VL comprises a sequence as set forth in SEQ ID NO: 106; or,
the VH comprises a sequence as set forth in SEQ ID NO: 78, and the VL comprises a sequence as set forth in SEQ ID NO: 106; or,
the VH comprises a sequence as set forth in SEQ ID NO: 79, and the VL comprises a sequence as set forth in SEQ ID NO: 106; or,
the VH comprises a sequence as set forth in SEQ ID NO: 80, and the VL comprises a sequence as set forth in SEQ ID NO: 106; or,
the VH comprises a sequence as set forth in SEQ ID NO: 81, and the VL comprises a sequence as set forth in SEQ ID NO: 106; or,
the VH comprises a sequence as set forth in SEQ ID NO: 82, and the VL comprises a sequence as set forth in SEQ ID NO: 106; or,
the VH comprises a sequence as set forth in SEQ ID NO: 83, and the VL comprises a sequence as set forth in SEQ ID NO: 106; or,
the VH comprises a sequence as set forth in SEQ ID NO: 84, and the VL comprises a sequence as set forth in SEQ ID NO: 106; or,
the VH comprises a sequence as set forth in SEQ ID NO: 85, and the VL comprises a sequence as set forth in SEQ ID NO: 106; or,
the VH comprises a sequence as set forth in SEQ ID NO: 86, and the VL comprises a sequence as set forth in SEQ ID NO: 106; or,
the VH comprises a sequence as set forth in SEQ ID NO: 87, and the VL comprises a sequence as set forth in SEQ ID NO: 106; or,
the VH comprises a sequence as set forth in SEQ ID NO: 88, and the VL comprises a sequence as set forth in SEQ ID NO: 106; or,
the VH comprises a sequence as set forth in SEQ ID NO: 89, and the VL comprises a sequence as set forth in SEQ ID NO: 106; or,
the VH comprises a sequence as set forth in SEQ ID NO: 90, and the VL comprises a sequence as set forth in SEQ ID NO: 106; or,
the VH comprises a sequence as set forth in SEQ ID NO: 91, and the VL comprises a sequence as set forth in SEQ ID NO: 106; or,
the VH comprises a sequence as set forth in SEQ ID NO: 92, and the VL comprises a sequence as set forth in SEQ ID NO: 106; or,
the VH comprises a sequence as set forth in SEQ ID NO: 93, and the VL comprises a sequence as set forth in SEQ ID NO: 106; or,
the VH comprises a sequence as set forth in SEQ ID NO: 94, and the VL comprises a sequence as set forth in SEQ ID NO: 106; or,
the VH comprises a sequence as set forth in SEQ ID NO: 95, and the VL comprises a sequence as set forth in SEQ ID NO: 106; or,
the VH comprises a sequence as set forth in SEQ ID NO: 96, and the VL comprises a sequence as set forth in SEQ ID NO: 106; or,
the VH comprises a sequence as set forth in SEQ ID NO: 97, and the VL comprises a sequence as set forth in SEQ ID NO: 106; or,
the VH comprises a sequence as set forth in SEQ ID NO: 98, and the VL comprises a sequence as set forth in SEQ ID NO: 106; or,
the VH comprises a sequence as set forth in SEQ ID NO: 99, and the VL comprises a sequence as set forth in SEQ ID NO: 106; or,
the VH comprises a sequence as set forth in SEQ ID NO: 100, and the VL comprises a sequence as set forth in SEQ ID NO: 106; or,
the VH comprises a sequence as set forth in SEQ ID NO: 101, and the VL comprises a sequence as set forth in SEQ ID NO: 106.

5. The isolated antigen-binding protein according to claim 1, wherein the isolated antigen-binding protein comprises the VH as set forth in SEQ ID NO: 70 and the VL as set forth in SEQ ID NO: 106.

6. The isolated antigen-binding protein according to claim 1, wherein the isolated antigen-binding protein comprises a heavy chain constant region derived from IgG1, IgG2, IgG3, or IgG4.

7. The isolated antigen-binding protein according to claim 1, wherein the isolated antigen-binding protein comprises an Fc constant region as set forth in SEQ ID NO: 320 or SEQ ID NO: 321.

8. The isolated antigen-binding protein according to claim 1, wherein the isolated antigen-binding protein comprises a light chain constant region derived from Igκ or Igλ.

9. The isolated antigen-binding protein according to claim 1, wherein the isolated antigen-binding protein comprises a light chain constant region as set forth in SEQ ID NO:318.

10. The isolated antigen-binding protein according to claim 1, wherein the isolated antigen-binding protein comprises a heavy chain as set forth in SEQ ID NO: 339 and a light chain as set forth in SEQ ID NO: 340.

11. A composition comprising the isolated antigen-binding protein according to claim 1 and a pharmaceutical acceptable carrier.

12. A method for treating a TL1A-mediated disease, comprising administering a subject in need thereof the isolated antigen-binding protein according to claim 1.

13. The method of claim 12 wherein the TL1A-mediated inflammatory disease is inflammatory bowel disease.

14. The method of claim 13, wherein the inflammatory bowel disease includes Ulcerative colitis or Crohn's disease.
